(19) United States
(12) Patent Application Publication
(10) Pub. No.: US 2012/0214173 A1
(43) Pub. Date: Aug. 23, 2012

(54) Methods of Identifying Modulators of an Ehd Polypeptide

(76) Inventors: Harvey McMahon, Cambridge (GB); Gary Doherty, Garvagh (GB); Richard Lundmark, Umea (SE); Oli Daumke, Berlin (DE)

(21) Appl. No.: 12/680,347

(22) PCT Filed: Oct. 1, 2008
(86) PCT No.: PCT/GB08/03355

§ 371 Date: Mar. 26, 2010

(30) Foreign Application Priority Data
Oct. 2, 2007 (GB) 0719277.6

(51) Int. Cl. G01N 33/53

(52) U.S. Cl. 435/7.8

(57) ABSTRACT

The invention relates to a method of identifying a modulator of an EHD family polypeptide, said method comprising (i) providing a first and second sample of an EHD polypeptide; (ii) contacting said first sample with a candidate modulator; (iii) contacting said first and second samples with ATP; and (iv) monitoring ATP hydrolysis in said first and second samples, wherein a difference between the ATP hydrolysis in said first and second samples identifies said candidate modulator as a modulator of an EHD family polypeptide. The invention also relates to methods of modelling molecules and to various EHD mutant polypeptides.

### FIELD OF THE INVENTION

The invention relates to the field of EHD polypeptides and their structure and/or biological function(s).

### BACKGROUND TO THE INVENTION

The ability to actively remodel membranes in response to nucleotide hydrolysis has largely been attributed to GTPases of the dynamin superfamily, and these have been extensively studied¹. Epsin homology (EH) domain-containing proteins (EHDs/Rme-1/pincher) comprise a less characterised class of highly conserved eukaryotic ATPases implicated in clathrin-independent endocytosis², and recycling from endosomes^{3,4}.

The dynamin superfamily of large GTPases are multi-domain proteins that include the classical dynamins (Dyn1, Dyn2, Dyn3), dynamin-related proteins (Mx proteins, Dlp, OPA and mitofusins) and the GBP/atlastin family¹. The proteins have an amino-(N-)terminal guanine nucleotide binding domain (G-domain) with a low affinity for nucleotides which is followed by a helical (or middle) domain. Additional domains are involved in membrane-binding and recruitment to sites of activation. Dynamin is the best characterised member where oligomerisation-stimulated GTP hydrolysis has been proposed to lead to scission of clathrin-coated vesicles⁵⁻⁸. Other superfamily members can function in membrane tubulation and membrane scission or fusion.

EHDs comprise a highly conserved eukaryotic protein family with four members (EHD1-4) in mammals and a single member in C. elegans, D. melanogaster and many eukaryotic parasites such as Plasmodium, Leishmania and Entamboeba. The proteins have a molecular mass of approximately 60 kD and contain an N-terminal G-domain, followed by a helical domain and a C-terminal EH-domain (FIG. 1a), although in plant homologues the EH-domain is N-terminal. The EH-domain is known to interact with asparagine-proline-phenylalanine (NPF) motifs in proteins involved in endocytosis.

EHD polypeptide structure is poorly understood in the art. EHD polypeptides are regarded as GTPases in the art. EHD biological functions are largely unknown in the art. The macromolecular behaviour of EHD polypeptides is incompletely understood in the art. The design or selection/screening for inhibitors or activators of EHD is not possible based on the inadequate information regarding the structure/function of EHD in the art.

The present invention seeks to overcome problem(s) associated with the prior art.

### SUMMARY OF THE INVENTION

EHD proteins are known by sequence analysis to contain a guanine nucleotide binding domain (G-domain). It has been suggested that EHD might bind to adenine nucleotides, with the attendant possibility that such nucleotides might be hydrolysed by the protein. However, there has been no accurate scientific study to date which reliably attributes ATP hydrolysis to a G domain. In the prior art, cross-nucleotide activities of that nature are typically attributed to contamination effects. The prior art view is very clearly established that G-domains have guanine nucleotide binding and/or guanine nucleotide hydrolysis activities.

The present inventors have crystallised and have studied the structure of EHD2 in unprecedented detail. This has led to a number of structural and mechanistic insights into the biology of EHD family polypeptides. A key finding is that EHD family polypeptides are in fact ATP binding polypeptides. Furthermore, we disclose how ATP binds to those polypeptides. In addition, the structural insights allowed a modelling of the likely mechanism of ATP hydrolysis by EHD, family polypeptides, which hypothesis has been demonstrated to be accurate by mutational studies of the ATPase activity.

The invention is based on these surprising findings.

In one aspect, the invention relates to a method of identifying a modulator of an EHD family polypeptide, said method comprising
(i) providing a first and second sample of an EHD polypeptide;
(ii) contacting said first sample with a candidate modulator;
(iii) contacting said first and second samples with ATP; and
(iv) monitoring ATP hydrolysis in said first and second samples,
   wherein a difference between the ATP hydrolysis in said first and second samples identifies said candidate modulator as a modulator of an EHD family polypeptide.

Suitably if hydrolysis of ATP is greater in said first sample than in said second sample then the candidate modulator is identified as an enhancer of EHD family polypeptide activity.

Suitably if hydrolysis of ATP is lower in said first sample than in said second sample then the candidate modulator is identified as an inhibitor of EHD family polypeptide activity.

Suitably ATP hydrolysis is monitored in the presence of lipid. Suitably said lipid is in the form of liposomes. Suitably said lipid is in the form of phosphatidylserine (PS) at a final concentration of about 10%.

In another aspect, the invention relates to a method as described above further comprising the step of providing a further sample of EHD family polypeptide, said further sample comprising an EHD family polypeptide bearing a T94A mutation, said further sample being used to determine the reference or background level of spontaneous ATP hydrolysis.

In another aspect, the invention relates to a method as described above further comprising the step of providing a further sample of EHD family polypeptide, said further sample comprising an EHD family polypeptide bearing an I157Q mutation, said further sample being used to determine the reference level of ATP hydrolysis in the absence of lipids.

In another aspect, the invention relates to a method as described above further comprising the step of providing a further reference sample, said further reference sample comprising a dynamin polypeptide together with GTP nucleotide and candidate modulator, said further sample being used to determine whether the candidate modulator has an EHD-specific effect, or whether it is also capable of affecting dynamin GTPase activity.

In another aspect, the invention relates to a crystalline EHD family polypeptide, said polypeptide being bound to an adenosine nucleotide or an analogue thereof.

In another aspect, the invention relates to a EHD polypeptide having the structure defined by the structural coordinates as shown in Table A.

In another aspect, the invention relates to a method for identifying a candidate modulator of EHD family polypeptide activity, said method comprising
(i) providing a molecular modelling apparatus with a set of structural coordinates of an EHD family polypeptide selected from those shown in Table A;
(ii) providing said molecular modelling apparatus with a set of structural coordinates of a molecular entity of interest; and
(iii) determining whether the molecular entity of interest is expected to bind to or to affect the structure of said EHD family polypeptide,
wherein an expectation of binding or affecting the structure of said EHD family polypeptide identifies said molecular entity of interest as a candidate modulator of EHD family polypeptide activity.

A molecular modelling apparatus is suitably a computer programmed with the appropriate tools for molecular modelling. Suitable programs/tools are noted in the examples section.

Suitably the structural coordinates of at least the EH-domain are selected. The first crystal structure of an EH domain is presented herein—in the prior art only low resolution NMR structural information has been available. The crystal structure of the EH domain enables it to be effectively targeted, for example to find or test inhibitors of the interaction with the NPF motifs of the target cargo.

Suitably the structural coordinates of at least the G-domain are selected.

Suitably the structural coordinates of at least the dimerisation interface are selected.

Suitably the structural coordinates of the oligomerisation interface are selected. In particular, reference is made to FIGS. 4a and b where key residues in oligomerisation are set out and in particular four residues are shown to abolish oligomerisation (E91Q, R167E, K193D, D198R). Thus, in modelling or studying the oligomerisation domain, attention should be advantageously focussed on those residues and their contribution to the structure.

Suitably the structural coordinates of at least the membrane binding site are selected.

The sites mentioned are described in more detail below, for example with reference to FIG. 1a.

Suitably each of the structural coordinates of Table A are selected.

In another aspect, the invention relates to a method for identifying a candidate therapeutic agent, said method comprising application of rational drug design to the crystal structure of EHD2.

Rational design of candidate agents likely to be able to interact with the target protein may be based upon structural studies of the molecular shapes of the target protein as disclosed herein. These will provide guidance as to which amino acid residues form molecular contact regions.

In another aspect, the invention relates to a method of manufacturing a modulator of an EHD family polypeptide, said method comprising identifying a candidate modulator as described above, and synthesising a quantity of said modulator.

The candidate therapeutic agent (or candidate modulator or molecular entity of interest (interchangeably referred to as ‘agent’ below)) may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule. The agent may be an antibody. The agent may be designed or obtained from a library of compounds, which may comprise peptides, as well as other compounds, such as small organic molecules. By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant' agent, an antibody, a natural or a non-natural agent, a fusion protein or equivalent thereof and mutants, derivatives or combinations thereof). Typically, the agent will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agent comprises at least the one of said cyclic groups linked to another hydrocarbyl group. The agent may be in the form of a pharmaceutically acceptable salt—such as an acid addition salt or a base salt—or a solvate thereof, including a hydrate thereof. For a review on suitable salts see Berge et al, (1977) J. Pharm. Sci. 66, 1-19.

In another aspect, the invention relates to use of a candidate modulator of EHD family polypeptide activity identified as described above, wherein said EHD family polypeptide is EHD2, in the manufacture of a medicament for diabetes.

In another aspect, the invention relates to use of the atomic coordinates as shown in Table A in the modelling of an EHD family polypeptide.

In another aspect, the invention relates to a method for the design of one or more ligands of an EHD family polypeptide, said method comprising the use of coordinates as shown in Table A.

In another aspect, the invention relates to use of an EHD family polypeptide in the tubulation of a biological membrane.

In another aspect, the invention relates to a method of tabulating a biological membrane comprising contacting said membrane with an EHD family polypeptide.

In another aspect, the invention relates to a method as described above or a use as described above wherein said membrane is comprised by a liposome. Suitably said membrane comprises phosphatidylserine (PS).

In another aspect, the invention relates to use of an EHD family polypeptide in membrane scission.

In another aspect, the invention relates to a method of inducing membrane scission, said method comprising contacting said membrane with an EHD family polypeptide. Suitably said method further comprises contacting said membrane-EHD family polypeptide complex with nucleotide in conditions permissive of nucleotide hydrolysis. Suitably said nucleotide is adenosine triphosphate (ATP).

Mutating has it normal meaning in the art and may refer to the substitution or truncation or deletion of the residue, motif or domain referred to. Mutation may be effected at the polypeptide level e.g. by synthesis of a polypeptide having the mutated sequence, or may be effected at the nucleotide level e.g. by making a nucleic acid encoding the mutated sequence, which nucleic acid may be subsequently translated to produce the mutated polypeptide. Where no amino acid is specified as the replacement amino acid for a given mutation site, suitably alanine (A) is used.

In another aspect the invention relates to a method of inhibiting dimerisation of an EHD family polypeptide, said method comprising mutating amino acid W238 of said polypeptide. In another aspect, the invention relates to a method of inhibiting dimerisation of an EHD family polypeptide, said method comprising mutating amino acids neighbouring W238 of said polypeptide, such as amino acids within 10 residues either side of W238, or amino acid residues within 5 residues either side of W238, or the immediate neighbouring residues of W238 (i.e. residues 237 and/or 239).

In another aspect, the invention relates to a method of modifying an EHD family polypeptide to permit guanine nucleotide binding thereto, said method comprising mutating said EHD polypeptide at one or more amino acid residues within the region H192 to M223, wherein said mutation alleviates steric exclusion of an amino group at carbon 2 of said guanine nucleotide.

In another aspect, the invention relates to a method of modifying an EHD family polypeptide to reduce or prevent membrane binding, said method comprising mutating any of K324, K327, K328, K329, K334, K341, V321 or F322. Suitably amino acid V321 and/or F322 is mutated—these are considered to have similar effects.

In another aspect, the invention relates to a method of modifying an EHD family polypeptide to reduce or prevent membrane binding, said method comprising mutating any of K324, K327, K328, K329 or F322. Suitably amino acid F322 is mutated.

In another aspect, the invention relates to a method of modifying an EHD family polypeptide to reduce or prevent ATP hydrolysis by said polypeptide, said method comprising mutating amino acid T72A or T94 of said polypeptide, suitably T94.

In another aspect, the invention relates to a method of modifying an EHD family polypeptide to reduce or prevent breakdown of membrane structures by said polypeptide, said method comprising mutating amino acid T72A or T94 of said polypeptide, suitably T94.

In another aspect, the invention relates to a method of modifying an EHD family polypeptide to increase ATP hydrolysis, such as to increase intrinsic ATP hydrolysis, by said polypeptide, said method comprising mutating amino acid I157 of said polypeptide.

In another aspect, the invention relates to a method of modifying an EHD family polypeptide to enhance breakdown of membrane structures by said polypeptide, said method comprising mutating amino acid I157 of said polypeptide.

Suitably said I157 is mutated to N, R or Q. Suitably said I157 is mutated to Q.

In another aspect, the invention relates to a method of inducing membrane fission, said method comprising contacting said membrane with an EHD family polypeptide comprising an I157Q mutation.

In another aspect, the invention relates to a method of modifying an EHD family polypeptide to reduce or abolish assembly stimulated ATP hydrolysis, said method comprising mutating said EHD polypeptide at E91, R167, K193, D198, F122, F128, or by deletion of the EH domain. Suitably said mutation(s) comprise one or more of the mutations set out in FIG. 4b.

In another aspect, the invention relates to an EHD family polypeptide or fragment thereof comprising one or more of the following mutations:
(i) T94A;
(ii) I157Q; and
(iii) F322A.

A fragment is suitably at least 10 amino acids in length, suitably at least 25 amino acids, suitably at least 50 amino acids, suitably at least 100 amino acids, suitably at least 200 amino acids, suitably the majority of the EHD polypeptide of interest. Suitably a fragment comprises a whole motif or a whole domain of the EHD polypeptide of interest. Suitably a fragment comprises at least 10 amino acids either side of the given mutation of interest. When more than one mutation is discussed, suitably a fragment comprises at least 10 amino acids each side of the two or more mutations, and suitably further comprises the internvening amino acid sequence too. Where a mutation is within 10 amino acids of the end of the polypeptide then suitably the fragment comprises the amino acids between said mutation and said end (e.g. the N- or C-terminus).

In another aspect, the invention relates to a method of destabilising a membrane, said method comprising contacting said membrane with a EHD family polypeptide and a nucleotide under conditions permissive of nucleotide hydrolysis.

In another aspect, the invention relates to an EHD family polypeptide comprising one or more of the mutations described herein.

Suitably said EHD family polypeptide or fragment thereof is or is derived from mammalian EHD2.

### DETAILED DESCRIPTION OF THE INVENTION

The invention provides architectural and mechanistic insights into an EHD ATPase involved in membrane remodeling. Here we show that epsin homology (EH) domain-containing proteins (EHDs) share many common features with the dynamin superfamily such as low affinity to nucleotides, the ability to tubulate liposomes in vitro, to oligomerise around lipid tubules in ring-like structures and to stimulate nucleotide hydrolysis in response to lipid binding. We present the structure of EHD2, bound to a non-hydrolysable ATP analogue, and provide evidence that EHDs are involved in nucleotide-dependent membrane scission in vivo. The nucleotide-binding domain is involved in dimerisation which creates a highly curved membrane-binding region. Oligomerisation of dimers occurs on another nucleotide-binding domain interface, and this allows us to model the EHD oligomer. We discuss the functional implications of the EHD2 structure for an understanding of membrane deformation.

Dynamins are distinguished from classical signalling GTPases by their large size, their low affinity for nucleotide and assembly-stimulated nucleotide hydrolysis. From studies of GBP1, dynamin, bacterial dynamin-like protein, and particularly the insights into EHD disclosed herein, it appears that the mechanism of assembly-stimulated nucleotide hydrolysis is also conserved. In cases where data are available assembly involves the same conserved interface in the G-domain, with the same orientation of G-domains, a phosphate cap and an activation mechanism which is dependent on a catalytic serine or threonine from switch I. This mechanism is different for the signal-recognition particle and its receptor where the nucleotides are found anti-parallel in the dimer and GTP hydrolysis involves catalysis by the 2′-hydroxyl group of opposing nucleotides. This assembly-stimulated GTP hydrolysis mechanism has likely been maintained across the dynamin superfamily.

We disclose the first molecular understanding of two separate interfaces in the G-domain which allow us to propose how oligomers are assembled. The structure, the architecture of the membrane interaction site and the proposed oligomerisation mechanism disclosed herein each provide an initial framework to understand the membrane remodelling function for the EHD family. EHD2 initially interacts with membrane via ionic interactions, and we disclose that the insertion of the conserved F322 at the tip of the helical domain will induce local curvature stress in the membrane. Furthermore, the highly curved membrane interaction site in the EHD oligomer is along the flat dimension of the lipid tubule, perpendicular to its curvature (see examples and FIG. 13b), and therefore EHD2 binding will create additional curvature stress on the membrane. Both factors are likely to contribute to membrane destabilisation, a prerequisite for membrane fission and fusion. We also disclose that nucleotide hydrolysis is most likely leading to membrane scission in vivo, and thus conformational changes induced by nucleotide hydrolysis are leading to further membrane destabilisation.

In another aspect, the invention may be useful in the attachment of entities to biological membranes. For example, the invention may relate to a method of attaching an entity to a biological membrane, the method comprising attaching said entity to a membrane binding element of an EHD family protein, and contacting the resulting complex with a biological membrane.

The invention may involve use of an EHD family polypeptide in the hydrolysis of ATP.

A biological membrane is typically a lipid bilayer membrane. An example of a biological membrane is a plasma membrane. More in particular, the biological membranes of the invention are often intracellular membranes, for example those involved in vesicle trafficking, or those forming a part of the endocytic recycling compartment. The term liposome has its normal meaning in the art, namely a single or multi laminar vesicle. Liposomes may be made from lecithins or other lipids. Preferably liposomes are made from brain derived lipids. Preferably liposomes are made from Folch extract. In some embodiments, liposomes may be made from 100% anionic phosphatidyl serine (PS) liposomes (it is to be noted that this 100% refers to the composition of the liposomes in this particular embodiment and should not infer the proportion of lipid present overall which is discussed elsewhere herein). Suitably liposomes may contain phosphatidyl inositol 4,5 bisphosphate (PIP2).

When the invention that is used in the tubulation of liposomes, preferably this takes place in vitro.

EHD Family Polypetides

EHDs comprise a highly conserved eukaryotic protein family. EHDs have a molecular mass of approximately 60 KD. EHD proteins contain a G domain, a helical domain, and a EH domain. Typically these domains occur in the order N terminus—G domain—helical domain—EH domain—C terminus. However, it should be noted that in plant EHDs, the EH domain may be at the N terminus.

For a polypeptide to be considered as an EHD family polypeptide, it should possess one or more of the above characteristics. More suitably, it should possess sufficient sequence identity to EHD1/2/3/4 to be classified in the same molecular family. Most suitably, it should be a mammalian EHD polypeptide. Most suitably, it should comprise mammalian EHD2 amino acid sequence. In particular, an exemplary EHD family polypeptide has the sequence shown as mmEHD2 in FIG. 8.

When particular amino acid residues are referred to using numeric addresses, the numbering is taken using mouse EHD2 (mmEHD2) amino acid sequence as the reference sequence. This is to be used as is well understood in the art to locate the residue of interest. This is not always a strict counting exercise—attention must be paid to the context. For example, if the protein of interest such as human EHD2 is of a slightly different length, then location of the correct residue in the human sequence corresponding to (for example) T94 may require the sequences to be aligned and the equivalent or corresponding residue picked, rather than simply taking the 94^{th} residue of the sequence of interest. This is well within the ambit of the skilled reader. In the unlikely event that any further guidance is needed, reference is made to FIG. 8 which presents a comprehensive alignment of sequences of interest with the reference sequence mmEHD2 at the top which both illustrates the principle and provides a robust reference chart for ease of location of the correct residues.

It will be apparent to the skilled reader that the invention is exemplified predominantly by reference to EHD2. It should be noted that EHD2 exhibits high sequence homology with other EHD family polypeptides. Thus, in some aspects the invention relates to the use of EHD2 in the development of therapeutics for application to other EHD family proteins. In particular, the crystal structure of the ATPase domain of EHD2 complexed with a bound ligand is applicable across the EHD family polypeptides since those polypeptides are also regarded as ATPases (having previously been thought to be GTPases).

In some aspects of the invention, it may be desirable to employ a functional test as to whether or not a particular polypeptide is to be considered an EHD family polypeptide. In addition to, or instead of, the sequence based criteria set out above, the following functional criterion may also be used: EHD family polypeptides can rescue a C. elegans RME knockout. Thus, in order to determine whether or not a particular polypeptide is indeed to be considered an EHD family polypeptide, it may be tested whether or not that polypeptide can rescue a C. elegans RME knockout. If the knockout is rescued, the polypeptide may be regarded as an EHD family polypeptide.

Thus there is a high degree of homology between all four classes of EHD in terms of nucleotide/amino acid sequence, and all four can rescue a C. elegans mutant in the orthologue RME-1. The C. elegans EHD, Rme-1, has been shown to regulate the exit of transmembrane proteins from a tubular intracellular compartment, the so-called endocytic recycling compartment³, and a similar location and role has been demonstrated for mammalian EHD1^{4,9}. EHD4/Pincher is predominantly localised to the plasma membrane and is involved in the uptake of the TrkA receptor in response to nerve growth factor (NGF) stimulation^{2,10}. Overexpression of EHD4 leads to increased clathrin-independent macropinocytic endocytosis of TrkA in the presence of NGF². Other members of the family also mediate trafficking of various ligands and overexpression of these EHDs leads to their localisation on tubules inside cells^{4,11-14}.

Exemplary applications of the invention are shown in the following table:

| | | | Therapeutic |
|---|---|---|---|
| | Pathway/interaction | Therapeutic inhibition | activation |
| EHD1 | MHC 1 | Inhibit in | Promote |
| | | transplanted organ - | immunological |
| | | Protect transplant | targeting - |
| | | from autorejection | enhance |
| | | | immune |
| | | | response |
| EHD2 | GLUT4 | Diabetes | |
| EHD3 | transferrin | | |
| EHD4 | Macropinocytic | cancer | cancer |
| | NGF/TrkA | | Pain |
| ALL | Virus entry | | |
| EHDs | parasites | | |

EHD1 is involved in MHC class I recycling at the membrane (Caplan et al. (2002) A tubular EHD1-containing compartment involved in the recycling of major histocompatibility complex class I molecules to the plasma membrane. EMBO J. 21 p 2557-67). Overexpression of EHD1 increases recycling. By inhibiting EHD1 activity, for example in a transplanted organ, the transplant may be protected from effects of autorejection. Conversely, activation of EHD 1 activity may promote an immunological response.

Thus the invention relates to use of a candidate modulator of EHD family polypeptide activity identified as described above, wherein said EHD family polypeptide is EHD1, in the manufacture of a medicament for ameliorating or enhancing immune responses. Amelioration relates to inhibitors of EHD1, augmentation applies to activators of EHD1.

EHD 1 may have an involvement in cystic fibrosis.

EHD2 is known to be involved in insulin mediated GLUT4 transport to the membrane (Park et al. (2004) EHD2 interacts with the insulin-responsive glucose transporter (GLUT4) in rat adipocytes and may participate in insulin-induced GLUT4 recruitment. Biochemistry 43 p 7552-62). Thus inhibition of EHD2 provides therapeutic benefit in the treatment of diabetes such as type II diabetes. Thus the invention relates to use of a candidate modulator of EHD family polypeptide activity identified as described above, wherein said EHD family polypeptide is EHD2, in the manufacture of a medicament for diabetes.

EHD4 mediates pinocytic endocytosis of functionally specialised nerve growth factor (NGF)/neurotrophic tyrosine kinase receptor type 1 (TrkA) to endosomes and TrkA-erk5 mitogen-activated protein kinase signalling (Shao et al. (2002) Pincher, a pinocytic chaperone for nerve growth factor/TrkA signaling endosomes. J. Cell Biol. 157 p 679-691). Thus modulation of EHD4 has application in the treatment of pain. Thus the invention relates to use of a candidate modulator of EHD family polypeptide activity identified as described above, wherein said EHD family polypeptide is EHD4, in the manufacture of a medicament for pain. In another aspect, the invention relates to use of a candidate modulator of EHD family polypeptide activity identified as described above, wherein said EHD family polypeptide is EHD4, in the manufacture of a medicament for cancer.

All EHDs are involved in clathrin-independent endocytosis, which is also known to be a mechanism of entry for viruses into cells (Damm et al. (2005) Clathrin- and caveolin-1-independent endocytosis: entry of simian virus 40 into cells devoid of caveolae. J. Cell Biol. 168 p 477-488). Thus broad inhibition of the EHD class finds application in viral infections. Moreover, the EHD sequence is conserved from parasites to higher mammals such as humans. By application of the methods of the invention to the isolation of parasite specific inhibitors, for example by screening parasite orthologues alongside mammalian EHDs, treatments for infections may usefully be provided. Thus in another aspect the invention provides use of a candidate modulator of EHD family polypeptide activity identified as described above, in the manufacture of a medicament for viral or parasite infection.

### INDUSTRIAL APPLICATION

EHD1 is involved in the recycling of MHC class 1 molecules. Thus, modulation of EHD1 has application in immune modulation, for example in transplant patients or in graft versus host disease.

EHD4 is involved in the internalisation of the TrkA receptor. This receptor responds to nerve growth factor (NGF). Thus, modulation of EHD4 is relevant to the modulation of, or treatment of, pain. NGF is also implicated in the survival and homeostatic maintenance of neurons; thus modulation of EHD4, for example EHD4 response to NGF, finds application in the treatment of neurodegeneration.

EHD2 is important in diabetes. Thus, modulation of EHD2 is important for the treatment of diabetes. More particularly, inhibition of EHD2 is important for the treatment of diabetes. Thus, the present invention particularly concerns screening for or validation of EHD2 inhibitors. Furthermore, the invention relates to the use of EHD2 inhibitors in the treatment of diabetes. In one embodiment, the invention relates to the screening for compounds capable of inhibiting EHD2, and to the use of such inhibitors in the manufacture of a medicament for diabetes.

Assays

There are numerous biological activities ascribed to EHD family polypeptides for the first time by the present inventors. These include oligomerisation (including dimerisation), lipid binding, nucleotide binding and nucleotide hydrolysis. The invention provides assays for modulators of one or more of these activities.

Regarding oligomerisation, without wishing to be bound by theory, this is a key target for therapeutic invention. The reason is believed to be that oligomerisation may provide specificity between different EHD family members such as EHD1/2/3/4. These individual proteins are known to localise to different places within the cell. Thus, by targeting the dimerisation or oligomerisation of those proteins, for example by targeting the oligomerisation/dimerisation interface defined by the present study, then an advantageous specificity can be provided for molecules so identified. Thus, suitably the assays of the present invention readout or screen for factors affecting oligomerisation.

It must be noted that oligomerisation facilitates lipid binding. Therefore, for certain assay readouts it may be possible that one or more underlying biological effects might be detected. For example, if the readout of a particular assay was lipid binding, this would be expected to be adversely affected by anything which inhibited oligomerisation. Such factors should be borne in mind by the skilled operator.

The assays of the invention suitably monitor EHD ATPase activity. Colorimetric ATPase assays are widely available commercially (e.g. http://www.innovabiosciences.com/products/atpase.php). The assay is suitably conducted according to the manufacturer's recommendations for ATPase assay. Suitably the assay may be conducted using regaents of InnovaBiosciences' catalogue number 601-0120. Suitably the assay is conducted essentially as set out in Innova Biosciences' Technical Bulletin number 654 (release 007; July 2005).

In vitro EHD polypeptides may exhibit a slow rate of ATP turnover—this may advantageously be enhanced by including a lipid preparation such as a liposome preparation into the assay of the invention.

Optionally, functional assay step(s) may be additionally used in order to better characterise the effect of the modulator(s) or treatment(s) being studied, or to verify the in vivo significance. An example of such an assay is to monitor internalisation of labelled NGF for candidate EHD4 modulators.

In some embodiments, it may be desirable to perform further assay steps in order to determine more precisely the biological effect of a given compound. For example, if it is suspected that a particular compound or treatment is affecting lipid binding, then a direct lipid binding assay might be employed. For example, a FRET based assay might be used. In this embodiment, a first FRET element would be attached to the EHD family polypeptide of interest, and a second FRET element would be attached to the lipid of interest. Thus, if the EHD family polypeptide does indeed bind the lipid then the FRET effect (fluorescence resonance energy transfer) permits this to be detected and thereby provides some insight as to whether or not lipid binding has been affected.

If it is suspected that a particular compound or treatment is having an effect on nucleotide binding by an EHD family polypeptide, this may be directly confirmed using a nucleotide binding assay. Any nucleotide binding assay known in the art may be employed for this subsequent step or assay, for example the direct binding of a radiolabelled or a fluorescently labelled nucleotide to the EHD family polypeptide of interest under the different treatments being investigated may be assessed.

In one embodiment of the invention, it is possible to apply molecular modelling aspects to the design of a nucleotide analogue. For example, it may be possible to design a nucleotide analogue which would selectively bind to an EHD family polypeptide such as EHD2. Thus, in some embodiments the invention relates to such design methods, and to a nucleotide analogue so designed.

It should be noted that the assays of the invention are likely to identify inhibitors of oligomerisation more often or more reliably than specific inhibitors of membrane binding. The reason is that the oligomerisation interface is much more extensive than the relatively much smaller interface which is believed to mediate membrane binding. Since disruption of oligomerisation leads to disruption of membrane binding, the hits which are detected by assaying for disrupted membrane binding would be expected to contain a greater proportion of hits disrupting oligomerisation and a smaller proportion of hits which directly interfere with the actual mechanism of membrane binding.

In some embodiments, it may be advantageous to employ an optional cell based step in the assay. For example, candidate modulators of EHD family polypeptides may be applied to cells harbouring a fluorescently labelled EHD family polypeptide. The EHD family polypeptide would normally be localised to one or more membrane locations within the cell, dependent on which EHD family member was being studied. If the presence of the candidate modulator changes the expected cellular distribution of the EHD family polypeptide being studied, then this is a dramatic indicator that membrane binding had been affected by that modulator. One example of an altered distribution would be a cytosolic distribution.

In one embodiment, the invention relates to a three step screening procedure having a first step of a ATPase screen, a second step of a cell based localisation screen, and an optional third step of a cargo internalisation screen. In another embodiment, the invention may relate to a two step procedure involving a first step of an ATPase screen and a second step of a cargo internalisation screen. In another embodiment, the invention may relate to a two step procedure involving a first step of an ATPase screen and a second step of a cell based localisation screen.

Dynamin Control

In some aspects of the invention, a dynamin control may be included as a reference sample in the assay of the invention. Using a dynamin family polypeptide as a control provides numerous advantages. Dynamin is one of the most homologous proteins to EHD in terms of sequence identity. It is expected that dynamin works via a similar mechanism for membrane scission. The activity and affinity profile for dynamin is thought to be similar to EHD. Furthermore, the lipid specificity of dynamin is very close to the specificity of EHD family polypeptides. Thus, by including dynamin as a parallel reference sample in the assays of the invention, false positives having a general effect (rather than an EHD specific effect) may beneficially be excluded from the screen at a very early stage.

It should be noted of course that dynamin is a GTPase. Therefore, the reference sample featuring dynamin should have GTP as a substrate, and should measure the hydrolysis of GTP (rather than ATP which will of course be used in assaying EHD activity). Suitably, two dynamin controls are used, one with candidate modulator and one without. This advantageously permits internal calibration of the background for dynamin, making the dynamin control more robust.

The candidate compounds or treatments of most interest will be those which have no effect on dynamin action, such as no effect on the GTP hydrolysis activity of dynamin, but which do affect EHD activity, such as ATP hydrolysis by EHD.

Inactive EHD Mutant

In some aspects of the invention, a control or reference sample is used which comprises an inactive EHD family polypeptide. Such a polypeptide is suitably constructed by mutation of the wild type EHD sequence. For example, the catalytic residue may be substituted, residues important in activation of hydrolysis may be substituted, residues involved in ATP binding may be substituted, or any other suitable alteration to the ATP hydrolytic elements of EHD may be made. Suitably the most stable polypeptide is selected as an inactive EHD mutant.

The most suitable EHD mutant to select is that which knocks out the ATPase activity.

A most suitable mutant is a T94A or T72A mutant, more suitably a T94A mutant, of an EHD family polypeptide (or the equivalent residue). This has the advantage of binding ATP but also has the advantage of being catalytically inactive, in other words the T94A mutant does not catalyse the hydrolysis of ATP. Design of this mutant has been enabled by the structural insights into EHD presented herein. The T94A mutant has the further advantage that it is exceptionally well suited to the search for factors affecting oligomerisation. This is because this mutant actually oligomerises slightly more readily even than the wild type EHD family polypeptide family itself.

Lipid/Membrane Component

The assays of the invention suitably comprise a lipid component such as a membrane component. The lipid component may comprise or contain any negatively charged lipid. The reason that the lipids should be negatively charged is due to the lysines present in the lipid binding site of EHD family polypeptides. Without wishing to be bound by theory, it is believed that the initial long range charge mediated interaction is between the negatively charged elements of the lipids and the positively charged lysine residues on the EHD family polypeptide.

Suitably the negatively charged lipid component may be phosphatidyl serine. Suitably the negatively charged lipid component may be any phosphatidyl inositol lipid. Most suitably, the negatively charged lipids may be provided in the form of liposomes such as Folch liposomes.

It should be noted that specificity for PIP2 (phosphatidyl inositol 4,5 bisphosphate) is observed for EHD family polypeptides. However, such specificity does not necessitate the use of PIP2. For example, if sufficient phosphatidyl serine (PS) is used, then it is not necessary to supply PIP2.

Any suitable concentration of negatively charged lipid may be used. Typically, lipid such as phosphatidyl serine (PS) is used at approximately 10%-20% final concentration.

I157Q

The invention provides a super-active EHD family polypeptide mutant. This mutant is suitably the I157Q mutant. EHD family polypeptides bearing this mutation are not well activated by lipids. In other words, EHD family polypeptides bearing the I157Q mutation are already very highly active in terms of ATP hydrolysis. By “not well activated by lipids” is meant that lipids are not required in order to reach the high level of activation observed. Thus, the present invention relates to an EHD family polypeptide bearing the I157Q mutation.

In addition, such mutants are useful in embodiments of the assay of the invention. For example, by employing the I157Q mutant as the EHD polypeptide in the assays of the invention, the inclusion of lipids in the assay may advantageously be avoided. In another embodiment, an I157Q EHD family polypeptide may be used as a reference or control sample. In this way, if a particular compound or treatment was shown to affect ATP hydrolysis of an EHD family polypeptide both with and without the I157Q mutation, in particular when lipids were absent from the I157Q assay sample, then this effectively provides another level of information about the action of the compound or treatment being studied. Specifically, if an I157Q mutant EHD is affected in a similar manner to a normal EHD polypeptide, then this would indicate that the action of the compound or treatment is via an ATP or an oligomerisation effect, and is far less likely to be via a lipid binding effect. Thus, the assays of the invention may employ I157Q mutants in order to distinguish between the effects on different biological aspects of the EHD family polypeptide being studied.

EHD3

EHD3 may be involved in transferrin uptake. Specifically, EHD3 may inhibit transferrin uptake. Transferrin uptake is an essential biological function, and therefore it is not desirable to interfere with this function. For these reasons, preferably inhibition of transferrin uptake by EHD3 is not a target of the present invention. However, the invention may be applied to the identification or development of inhibitors of EHD3 which still allow transferrin uptake. In another embodiment transferrin uptake may be used as a readout in a cellular screen to ensure EHD modulators do not interfere with this vital function (i.e. a counter screen).

Structure Based Design

Determination of the 3D structure of EHD provides important information about the likely active sites of EHD, particularly when comparisons are made with similar enzymes. This information may then be used for rational design of EHD inhibitors or interactors, e.g. by computational techniques which identify possible binding ligands for the active sites, by enabling linked-fragment approaches to drug design, and by enabling the identification and location of bound ligands using X-ray crystallographic analysis. These techniques are discussed in more detail below.

An iterative approach to ligand design based on repeated sequences of computer modelling, protein-ligand complex formation and X-ray analysis may be employed. EHD inhibitors may also be designed in the this way. More specifically, a ligand (e.g. a potential inhibitor) of EHD may be designed that complements the functionalities of the EHD active site(s) such as the oligomerisation site or ATPase site. The ligand can then be synthesised, formed into a complex with EHD, and the complex then analysed by X-ray crystallography to identify the actual position of the bound ligand. The structure and/or functional groups of the ligand can then be adjusted, if necessary, in view of the results of the X-ray analysis, and the synthesis and analysis sequence repeated until an optimised ligand is obtained. Related approaches to structure-based drug design are also discussed in Bohacek et al., Medicinal Research Reviews, Vol. 16, (1996), 3-50.

As a result of the determination of the EHD 3D structure, more purely computational techniques for rational drug design may also be used to design EHD inhibitors (or activators). For example, automated ligand-receptor docking programs (discussed e.g. by Jones et al. in Current Opinion in Biotechnology, Vol. 6, (1995), 652-656) which require accurate information on the atomic coordinates of target molecules may be used to design EHD inhibitors (or activators).

Linked-fragment approaches to drug design also require accurate information on the atomic coordinates of target molecules. The idea behind these approaches is to determine (computationally or experimentally) the binding locations of plural ligands to a target molecule, and then construct a molecular scaffold to connect the ligands together in such a way that their relative binding positions are preserved. The connected ligands thus form a potential lead compound that can be further refined using e.g. iterative technique(s). For a virtual linked-fragment approach see Verlinde et al., J. of Computer-Aided Molecular Design, 6, (1992), 131-147, and for NMR and X-ray approaches see Shuker et al., Science, 274, (1996), 1531-1534 and Stout et al., Structure, 6, (1998), 839-848. The use of these approaches to design EHD inhibitors is made possible by the determination of the EHD structure.

Many of the techniques and approaches to structure-based drug design described above rely at some stage on X-ray analysis to identify the binding position of a ligand in a ligand-protein complex. A common way of doing this is to perform X-ray crystallography on the complex, produce a difference Fourier electron density map, and associate a particular pattern of electron density with the ligand. However, in order to produce the map it is necessary to know beforehand the protein 3D structure (or at least the protein structure factors). Therefore, determination of the EHD structure also allows difference Fourier electron density maps of EHD-ligand complexes to be produced, which can greatly assist the process of rational drug design.

The approaches to structure-based drug design described above all require initial identification of possible compounds for interaction with the target molecule (in this case EHD). Sometimes these compounds are known e.g. from the research literature. However, when they are not, or when novel compounds are wanted, a first stage of the drug design program may involve computer-based in silico screening of compound databases (such as the Cambridge Structural Database) with the aim of identifying compounds which interact with the active site or sites of the target bio-molecule. Screening selection criteria may be based on pharmacokinetic properties such as metabolic stability and toxicity. However, determination of the EHD structure allows the architecture and chemical nature of each active site to be identified, which in turn allows the geometric and functional constraints of a descriptor for the potential inhibitor to be derived. The descriptor is, therefore, a type of virtual 3-D pharmacophore, which can also be used as selection criteria or filter for database screening.

The invention relates to the selection and/or design and/or screening for inhibitors or activators or molecules capable of interfering with or binding to EHD polypeptides. In particular the invention relates to screening for inhibitors of EHD polypeptides.

The invention is now described by way of example. These examples are intended to be illustrative, and are not intended to limit the appended claims. Reference is made to the following figures:

### BRIEF DESCRIPTION OF THE FIGURES

FIG. 1 shows that EHD2 shares common properties with the dynamin superfamily: a, Domain structure of EHD proteins. Amino acid numbering corresponds to mouse EHD2. b, EHD2 binds to adenine nucleotides, as determined by ITC. For clarity, ΔΔH=ΔHn−ΔH1 is plotted. Fitted values are: ATP-γ-S [symbol] : 13±3 μM (n=0.96), ADP [symbol] : 50±6 μM (n=0.9), GTP-γ-S [symbol] and EHD2(T72A)-ATP-γ-S [symbol] : no binding observed. c, Coomassie-stained gels of co-sedimentation assays in the presence of 1 mM ATP-γ-S using brain-derived Folch, 100% PS and synthetic liposomes (70% phosphatidyl-choline, 10% PS, 10% cholesterol, 10% of the indicated Pls). P-pelleted fraction, S-supernatant. d, Negative stain EM of PS liposomes in the absence (top) or presence (middle, bottom) of EHD2 and 1 mM ATP-γ-S. The bottom panel shows an intermediate in the tubulation process, surrounded by EHD2 rings of variable diameter. e, EGFP-tagged EHD2 was over-expressed in HeLa cells. Whereas the wild-type protein localised to puncta and tubular networks close to the cell surface, the T72A mutant was cytoplasmic. Images were acquired close to the basal cell surface. f, Nucleotide hydrolysis was measured by HPLC in the absence (empty symbols) or presence of Folch liposomes (filled symbols). Data represent mean and standard deviation for two (intrinisic reactions) or three (stimulated reactions) independent experiments. Whereas the intrinsic ATP reaction was 8-fold stimulated by Folch lipids [symbol] , GTP hydrolysis was not stimulated [symbol] . The nucleotide-free T72A mutant did not show stimulation of ATP hydrolysis [symbol] .

FIG. 2 shows the structure of EHD2: a, Ribbon-type presentation of the EHD2 dimer. Molecule A is coloured according to the secondary structure and molecule B according to the domain structure. Disordered loops are represented by dashed lines. The GPF motif in the linker regions is drawn in purple, the localisation of the KPFxxxNPF and further motifs in the G-domain are shown. b, Topology plot of EHD2 in which circles represent α-helices and triangles represent β-strands. Comparison with the classical Ras-like G-domain (boxed) and the G-domain of dynamin indicates that EHD2 has a dynamin-related switch I extension. c, Left Electrostatic surface representation of the EHD2 EH-domain in which red indicates negative charge and blue positive charge at neutral pH. F422 is penetrating in the non-charged peptide binding pocket. Right: Superposition of the EHD2 EH-domain (dark-green) with the second EH-domain of Eps15 (orange) bound to an NPF-containing peptide (pdb code 1FF1)²². Structure and peptide binding mode is very similar in both EH-domains. d, Comparison of the nucleotide-binding sites of Ras (pdb code 5p21, left) and EHD2 (right). Residues from the NKxD motif involved in specific nucleotide recognition are shown with hydrogen bonds indicated. In EHD2, D222 from the NKxD motif forms a salt bridge to R536 which is supplied from the opposing EH-domain (in green) into the nucleotide binding site.

FIG. 3 shows membrane binding and the role of ATP hydrolysis: a, Ribbon-type representation of the putative membrane-binding site with residues tested for membrane-binding in ball-and-stick representation. b, Coomassie-stained gels of sedimentation assays in the absence (lower panel) and presence of Folch liposomes using wild-type EHD2 and the indicated mutants. c, Nucleotide hydrolysis of the lipid binding mutants was carried out as described in FIG. 1f. The F322A mutant [symbol] intrinsic, ̂stimulated reaction) showed a 40% decrease in the stimulated ATPase reaction (kobs=3.0 h⁻¹ for F322A versus 5.4 h⁻¹ for wt), and the mutant K328D [symbol] a 75% reduced rate (kobs=1.6 h⁻¹). d, EGFP-tagged F322A mutant showed a completely cytoplasmic distribution when over-expressed in HeLa cells. Also mutations K324D, K327D, K328D and K329D led to a completely cytoplasmic distribution of EHD2 (data not shown). e, Affinity of the T94A mutant to ATP-γ-S was determined as described in FIG. 1b. Nucleotide hydrolysis was measured as described in FIG. 1f. In vitro tubulation activity of PS liposomes was analysed as described in FIG. 1d. f, EGFP-tagged mutants were over-expressed in HeLa cells. Whereas the T94A mutants showed extensive tubulation with no punctate stained, the I157Q mutant showed exclusively punctate staining. g, Quantification of the over-expression phenotypes from FIG. 3f. For each construct, three independent experiments with ≈50 cells/experiments were analysed.

FIG. 4 shows the EHD2 oligomer: a, The alignment in FIG. 8 was used to create a surface conservation plot of the EHD2 dimer (in the same orientation as in FIG. 2a) with conserved residues shown in purple and non-conserved residues in cyan. Conserved surface-exposed residues mutated in the following experiments are indicated. A highly conserved surface patch is present in the G-domain, in between the EHD2 monomers. b, Nucleotide hydrolysis for mutants in the proposed oligomerisation interface was measured as described in FIG. 1f. Note that mutant R167E (in Switch II) showed an increased basal ATPase activity which was not further stimulated by Folch liposomes. Furthermore, deletion of the EH-domain and the mutation of the side site xPFs motif completely prevented the membrane-stimulated ATPase reaction. c, Model of the EHD2 oligomer, Top: Surface representation (orange and blue-cyan dimer) and mesh representation (two dimers on the right) of the proposed oligomer. Only the EH-domains of the second dimer are shown in blue and cyan. The linker region and the disordered KPFxxxNPF loop are shown as dotted lines. The proposed movement of the EH-domains from the top site linker to the side. site G-domain are indicated by arrows. The two EHD2 monomers which were used for the generation of this model are shown in the dotted box with the AMP-PNP molecules facing each other in a head-to-head fashion. Bottom part: Side view of the oligomer. The membrane-binding sites are facing all in the same direction towards the putative membrane interface.

FIG. 5 shows expression and purification of mouse EHD2. Mouse EHD2 was expressed in Escherichia coli as a His-fusion protein as described in Materials and Methods. NI—Non-induced culture. I—Induced culture. SN—Soluble extract. FT—Soluble extract after application to NiNTA Sepharose. E1-EHD2 after elution from NiNTA-Sepharose. E2-EHD2 after dialysis and thrombin cleavage. E3-EHD2 after re-application and elution from the NiNTA column. This protein was further purified by size-exclusion chromatography using a Sephadex S200 column (data not shown).

FIG. 6 shows ultracentrifugation analysis indicates that EHD2 is a dimeric protein. Sedimentation velocity experiments were performed as described in methods. Selected scans (at equal, ˜15 min intervals), and of g(s20,w) (the amount of material sedimenting between s20,w and (s20,w+δs)) and also the residuals for fitting the data with DCDT+, were plotted with the program profit v.5.6.7 (Quantum soft, Switzerland). The fitted value is 113±4 kDa which corresponds well with the calculated mass of the dimer of 124 kDa.

FIG. 7 shows EHD2 tubulation of PS- and synthetic liposomes. EHD2 was incubated with the indicated liposomes in the presence and absence of nucleotides and analysed by EM as described in Methods. a, EHD2 deformed PS liposomes into tubular networks, here in the presence of ATP-γ-S. b, Enlarged views of the indicated area in a. Note the presence of regularly spaced EHD2 rings (some are indicated with arrows). This even spacing may be due to the curvature stress generated by an EHD2 ring along the axis of the lipid tubule which might disfavour binding of the next ring in the direct vicinity (see below). c, Some of the lipid tubules (especially at higher protein concentration) were tightly packed with EHD2 oligomeric rings. d,e In the presence of ADP and in the absence of nucleotide (f,g), EHD2 also tubulated PS liposomes and formed ring-like structures around the tubules. e,g are enlarged views of the indicated areas in d and f, respectively. We did not observe a noticeable change of size of the tubules with the different nucleotide conditions. h,i,j, Under less favourable lipid binding conditions (synthetic liposomes with no PS and only 2.5% PIP2), EHD2 tubulated liposomes in the presence of ATP-γ-S (h) and ADP (i), but we did not observe tubulation in the absence of nucleotides (j).

FIG. 8 shows alignment and secondary structure assignment. Multiple sequence alignment of the EHD family. The following sequences were aligned (expasy accession number in brackets): Mouse musculus EHD2 (Q8BH64), Homo sapiens EHD1 (Q9H4M9), Homo sapiens EHD3 (Q9NZN3), Homo sapiens EHD4 (Q9H223), Danio rerio EHD (Q6P3J7), Xenopus laevis EHD1 (Q7SYA1), Xenopus laevis EHD4 (Q7ZXE8), Drosophila melanogaster PAST-1 (Q8IGN0), Caenorhabditis elegans RME1 (Q966F0), Schistosoma japonicum EHD (Q5DG45), Dictyostelium discoideum EHD (Q54ST5), Plasmodium falciparum EHD (Q9NLB8), Entamoeba histolytica EHD (Q515L4), Trypanosoma cruzi EHD (Q4DYK9), Leishmania major EHD (Q4QDJ3S), Arabidopsis thaliana EHD (Q3EAA4). Completely conserved residues are boxed in red, conserved residues (>50% sequence identity) are boxed in green. Helices are indicated by cylinders, β-strands as blue arrows with colours according to the domain organisation (see FIG. 1a). Residues mutated in this study are indicated with a diamond (♦).

FIG. 9 shows the EHD dimer interface. The alignment in FIG. 8 was used to create a surface conservation plot of the EHD2 G-domain and helical domain, with conserved residues shown in purple and non-conserved residues shown in cyan. Helix α6 with its invariant W238 and the contiguous loop of the opposing EHD2 monomer are shown in orange interacting with the conserved surface.

FIG. 10 shows the Ca²⁺ binding site of the EH-domain. Residues involved in Ca²⁺-binding and W490 at the bottom of the peptide binding pocket are in dark grey and the bound GPF-peptide is in light grey. Five direct ligands for Ca²⁺ could be identified which are the carboxy side-chains of D494, D496, D498, E505 and one main chain oxygen from M500 (mc500). The sixth ligands might be a water molecule not seen at the current resolution.

FIG. 11 shows Top: Electrostatic surface representation of EHD2. Red indicates negative charge and blue positive charge at neutral pH. The orientation of EHD2 is the same as in FIG. 2a. The membrane interaction site is highly curved which is a consequence of EHD2 dimerisation. The diameter of the sphere is approximately 7 nm, but this may not be so extreme if the tips of the loops (a phenylalanine and a valine residue) are inserted into the membrane. Bottom: EHD binding to liposomes of different sizes in the presence and absence of 1 mM nucleotides and 1 mM MgCl2 (P—pelleted fraction, S supernatant). In the absence of nucleotide, the protein shows a strong binding preference for highly curved membranes. The liposomes have 87.5% phosphatidyl-choline, 10% cholesterol, 2.5% phosphatidyl-inositol(4,5)P2 and no added phosphatidyl-serine.

FIG. 12 shows the phosphate cap. The phosphates of the AMP-PNP molecule are covered by residues from switch I and the P-loop. This cap does not allow the introduction of a catalytic residue in trans.

FIG. 13 shows: a, Top and side view of the EHD2 oligomeric ring model in a surface representation. For better clarity, the EH domains are not included. The diameter of the embraced lipid tubule is ≈18 nm and the thickness of the EHD2 ring is ≈10 nm, in agreement to what is observed in the EM assays. Approximately twenty EHD2 dimers constitute one turn in this model. b, Arrangement of the EHD2 dimers in the oligomer. The high curvature of the membrane interaction site of EHD2 (FIG. 11) is oriented perpendicular to the curvature of the lipid tubule. This arrangement is predicted to induce local curvature on the lipid tubule. c, In an alternative model, the curvature of the EHD2 lipid interaction site matches the curvature of the lipid tubules. However, such an arrangement of the EHD2 oligomer was never observed in EM studies (FIG. 1d, FIG. 7).

FIG. 14 shows analysis of peptide binding to the EH-domain. The affinity of hepta-peptides from EHD2 containing the indicated xPF motifs to the EH-domain of EHD2 was measured by ITC at 10° C. in 100 mM HEPES (pH 7.5), 50 mM NaCl, 200 μM CaCl2. ¹¹⁸Peptides PEKPFRKL and ⁴¹⁸RMGPFVER did not (show detectable binding whereas binding of peptide ¹²⁴KLNPFGNT could be fitted to an affinity of 130±20 μM (n=1.33).

FIG. 15 shows a table.

### EXAMPLES

We disclose the structure and function of mouse EHD2 as a model for the EHD family. The following methods were generally applied:

ITC measurements were performed at 10° C. in 20 mM HEPES (pH 7.5), 300 mM NaCl, 2 mM MgCl2. Liposome binding assays were performed as described previously (www.endocytosis.org). Multiple turnover ATPase assays were performed in 20 mM HEPES (pH 7.5), 135 mM NaCl, 15 mM KCl, 1 mM MgCl2 at 30° C. with 10 μM EHD2 (or mutants) as enzyme and 100 μM ATP as substrate, in the absence or presence of 1 mg/ml Folch liposomes (Sigma-Aldrich).

Reactions were started by the addition of the protein to the final reaction mix and nucleotide hydrolysis was followed using standard HPLC measurement²⁷. Initial rates were determined by applying a linear fit to data points up to 40% nucleotide hydrolysis. For electron microscopic studies, 2.5 μM EHD2 in 20 mM HEPES (pH 7.5), 150 mM NaCl, 1 mM MgCl2 was incubated for 15 min at 25° C. in the presence of 1 mM nucleotide and 0.05 mg/ml (final concentration) of the indicated liposomes. Samples were spotted on carbon-coated copper grids (Canemco and Marivac) and negatively stained with 2% uranyl acetate.

Protein expression and structure determination. Mouse EHD2 full-length protein and all mutants were expressed as N-terminal His-fusions followed by a PreScission cleavage site in Escherichia coil BL21 DE3 Rosetta (Novagen) from a modified pET28 vector. Bacteria cultures in TB medium were induced at an OD of 0.2 with 40 μM IPTG and grown overnight at 18° C. Bacteria were lysed in lysis buffer containing 50 mM HEPES (pH 7.5), 400 mM NaCl, 25 mM Imidazole, 2.5 mM β-Mercaptoethanol (β-ME), 500 μM Pefablock S C (Boehringer Ingelheim) using an Emulsiflex homogeniser (Avestin, Canada). After centrifugation at 100,000 g for 45 min at 4° C., the soluble extract was applied to a NiNTA-column (Qiagen, Hildesheim) equilibrated with lysis buffer. The column was extensively washed with 20 mM HEPES (pH 7.5), 700 mM NaCl, 30 mM Imidazole, 2.5 mM β-ME, 1 mM ATP, 10 mM KCl and shortly with 20 mM HEPES (pH 7.5), 300 mM NaCl, 25 mM Imidazole, 2.5 mM β-ME. Bound protein was eluted with 20 mM HEPES (pH 7.5), 300 mM NaCl, 100 mM Imidazole, 2.5 mM β-ME and dialysed overnight at 4° C. against 20 mM HEPES (pH 7.5), 300 mM NaCl, 2.5 mM β-Mercaptoethanol in the presence of 250 μg PreScission protease to cleave the His-tag. The protein was re-applied to a NiNTA column to which it bound under these buffer conditions also in the absence of the His-tag. The column was extensively washed with 20 mM HEPES, 300 mM NaCl, 2.5 mM β-ME, and the protein finally eluted with 20 mM HEPES, 300 mM NaCl, 2.5 mM β-Me, 25 mM Imidazole, concentrated and further purified using a Sephadex200 size-exclusion column (two consecutive runs for proteins used for the ATPase assays). Typical yields were 4 mg purified EHD2/1 bacteria culture. At 300 mM NaCl we could concentrate the protein to 40 mg/ml but at lower salt concentration we observed some precipitation at this protein concentration. The protein was partially stabilised by 1 mM MgCl2.

Crystallisation and structure determination. For crystallisation, a selenomethioninesubstituted point mutant Q410A was prepared as described³⁰. This mutant showed identical biochemical properties as the wild-type protein but displayed less degradation in the linker region when incubated over longer periods of time. The protein was concentrated to 40 mg/ml and supplemented with 4 mM MgCl2, 2 mM AMP-PNP (Sigma-Aldrich, both final concentrations). The hanging-drop vapour-diffusion method was used for crystallisation. 2 μl protein solution were mixed with an equal volume of reservoir solution containing 3% PEG2000 MME, 50 mM MES (pH 6.4), 4 mM MgCl2. Crystals appeared after one week at 4° C. and had dimension of ≈0.2×0.2×0.05 mm³. For flash-freezing in liquid nitrogen, they were first transferred for 10 sec in 50 mM MES (pH 6.4), 75 mM NaCl, 4 mM MgCl2, 2 mM AMP-PNP, 14% MPD before incubation in the final cryo-solution containing 50 mM MES (pH 6.4), 75 mM NaCl, 4 mM MgCl2, 2 mM AMP-PNP, 27% MPD. No crystals were obtained in the presence of ADP or in nucleotide-free conditions.

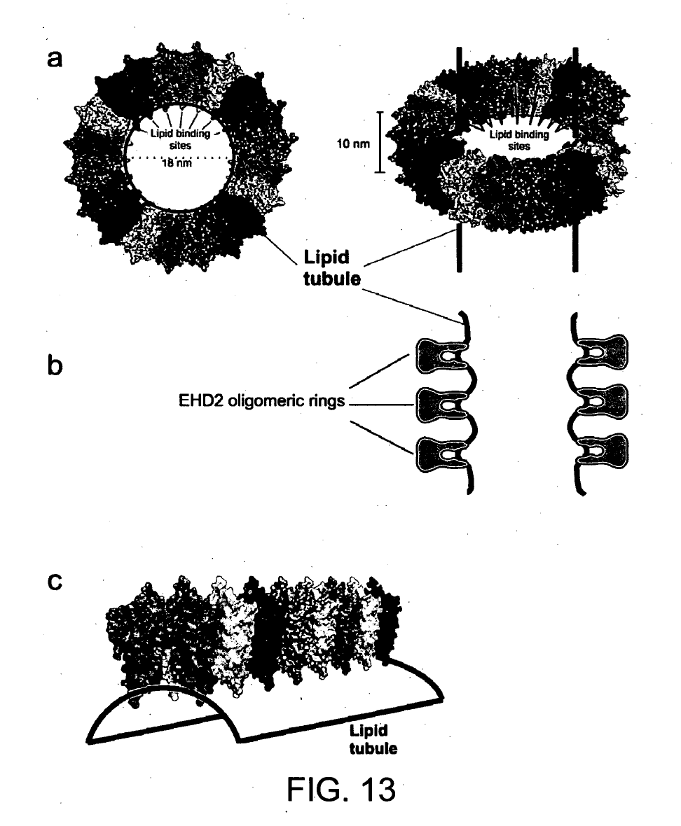

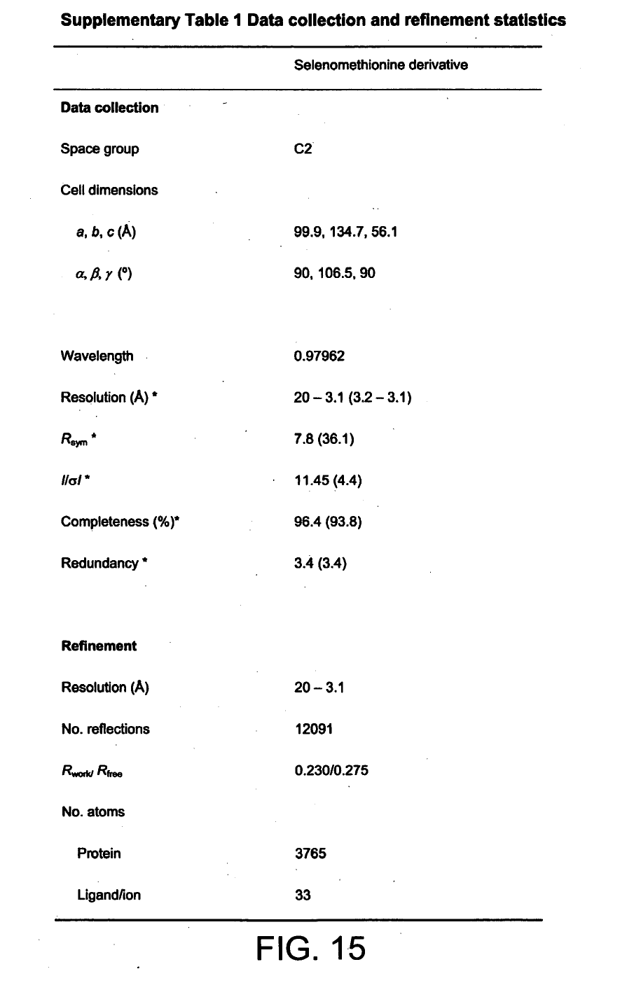

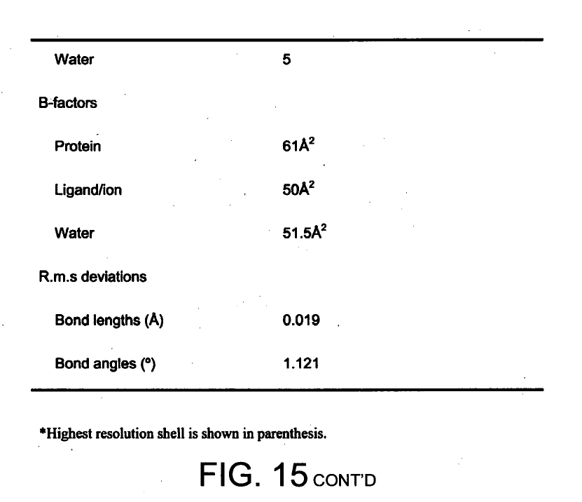

One dataset at the selenium peak wavelength was collected from a single crystal at the ESRF beamline ID14-EH4 (see FIG. 15 (“Supplementary Table 1”)) and processed and scaled using the xds program suite³¹. Crystals belonged to the monoclinic crystal system and contained one molecule in the asymmetric unit. 13 out of 16 selenium atoms were found from SHELXD³² using the anomalous signal of the dataset. Selenium sites were refined and initial phases were calculated using the program SHARP³³. In the resulting electron density, the main chain was clearly traceable, and an initial model could be built using the XtalView package³⁴. The model was refined using Refmac5³⁵ with 3 TLS groups (FIG. 15 (“Supplementary Table 1”)). The asymmetric unit contains 477 amino acids, one AMPPNP, one magnesium, one calcium and five water molecules and has an excellent geometry with all residues in the favoured and most favoured region of the Ramachandran plot as judged by the program Procheck³⁶. Ribbon plots were prepared using the program Molscript³⁷ and rendered with Raster3D³⁸. Surface conservation plots were prepared using the ConSurf server³⁹ and ccp4 molecular graphics⁴⁰. Electron potential maps were generated using ccp4 molecular graphics. All other surface representations were prepared using pymol⁴¹. To predict the arrangement of the EHD2 dimer in the oligomer, two EHD2 dimers were superimposed with one of the two monomers of the GBP1.GDP.AlF3-dimer (pdb code 2B92) using swisspdb viewer⁴². The EHD2 dimers were manually re-aligned to avoid amino acid clashes in a way that the two-fold axis between the oligomerising EHD2 monomers was maintained. A high degree of shape complementarity between the EHD2 dimers in the resulting tetramer supported this approach (FIG. 13). Furthermore, the lipid binding sites of both EHD2 dimers are expected to contact the membrane, and this restraint is fulfilled in the tetramer. To obtain a 20 nm ring, an 18° tilt was introduced between the dimers. We refrained from energy minimising of this structure since major conformational changes in the interface are expected to take place upon oligomerisation (ordering of switch I and switch II) and since the resolution of the structure is not appropriate for an accurate prediction. The programs superpose and pdbset from ccp4⁴³ were used to generate the oligomer from the tetramer. Pdb coordinates of the proposed oligomer are found in the Supplementary Materials.

Ultracentrifugation. Sedimentation velocity experiments were performed in a Beckman Optima XLA ultracentrifuge, using an An-60Ti rotor. Centrifugation was at 50,000 rev×min and 5° C. at an EHD2 concentration of 15 μM, with scans as fast as possible (˜1.5 min intervals). The data were analysed using DCDT+ v.2^{44,45}, with the partial specific volume for the protein (from the amino acid composition) and solvent density and viscosity calculated using Sednterp⁴⁶. Selected scans (at equal, ˜15 min intervals), and of g(s20,w) (the amount of material sedimenting between s20,w and (s20,w+δs)) and also the residuals for fitting the data with DCDT+, were plotted with the program profit v.5.6.7 (Quantum soft, Switzerland). Cell biology. N-terminal EGFP-tagged EHD2 and all mutants were over-expressed in HeLa cells from the pEGFP-C3 vector (Clontech). HeLa cells were grown as per ECACC guidelines with 10% fetal bovine serum and transfected using Genejuice (Novagen) for transient protein expression. 24 h after transfection, cells were fixed for 10 min at 37° C. in 3% paraformaldehyde and mounted. All confocal images were taken sequentially using a BioRad Radiance system and LaserSharp software (Biorad). For real-time microscopy, transfected cells on glass-bottom Petri dishes (WillCo Wells BV, Amsterdam) were washed with 25 mM HEPES (pH 7.5), 125 mM NaCl, 5 mM KCl, 10 mM D-glucose, 1 mM MgCl2, 2 mM CaCl), and epifluorescence images were taken using an Olympus IX70 microscope (Southhall, UK) and Argon laser (Melles Griot, Carlsbad, Calif.) with a Princeton instruments (Trenton, N.J.) cooled I-PentaMAX camera with MetaMorph software (Universal imaging).

### Example 1

### Expression and Purification

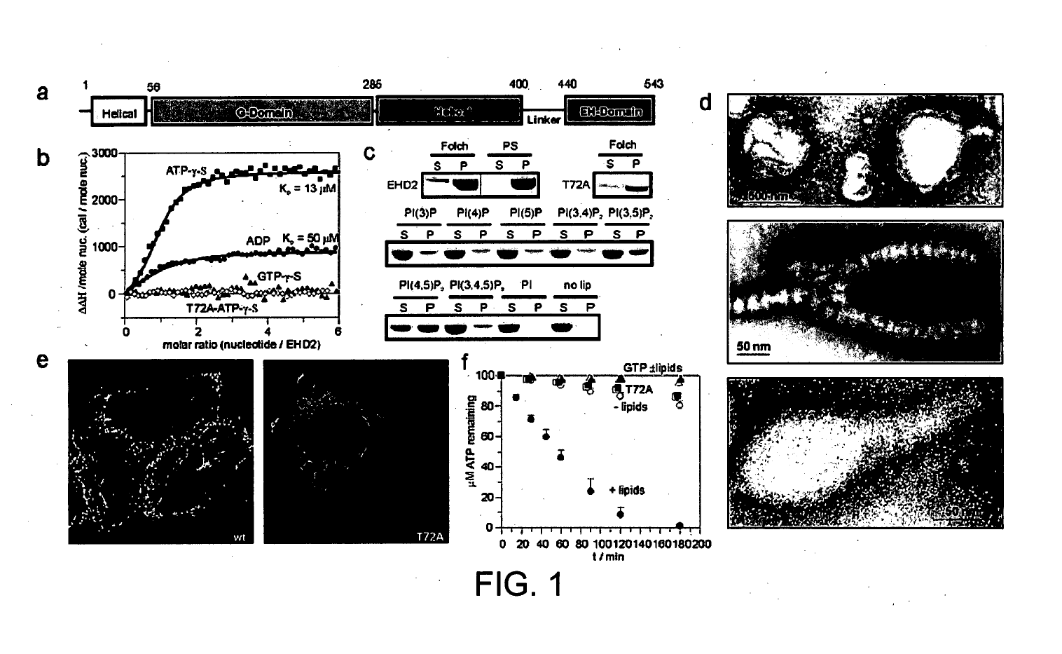

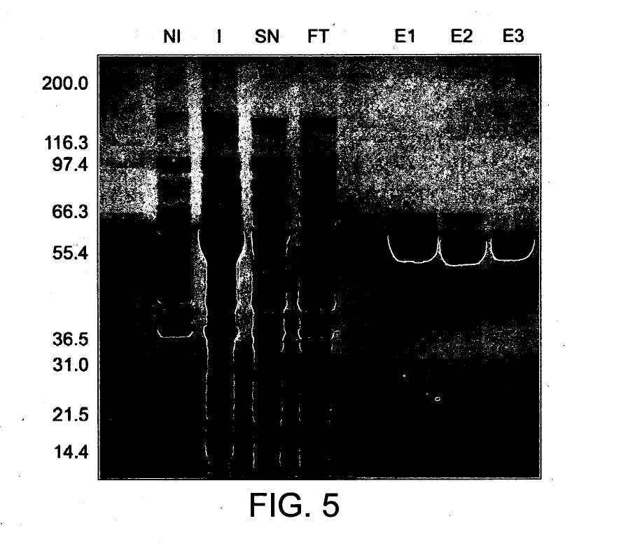

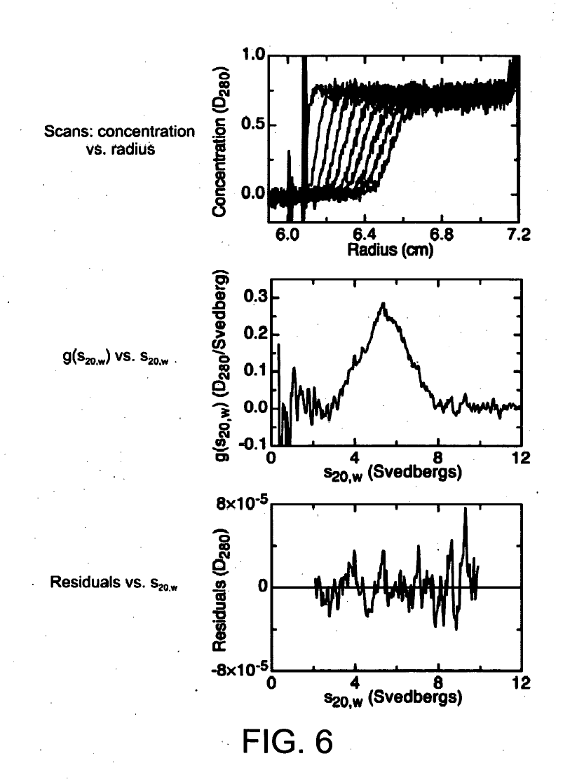

Mouse full-length EHD2 was expressed in bacteria and purified to homogeneity (FIG. 5). The purified protein was nucleotide-free as judged by HPLC analysis. In 300 mM NaCl, EHD2 was highly soluble, eluted as a dimeric protein by size-exclusion chromatography, and was found to be a dimer in dynamic light scattering experiments and in analytical velocity centrifugation (FIG. 6). At 50 μM protein concentration, the hydrodynamic radius did not change in the presence or absence of nucleotides (or in 150 mM versus 300 mM NaCl), as judged by dynamic light scattering experiments. It was previously reported that EHDs—despite having a predicted G-domain—bind to adenine nucleotides¹⁵. We confirmed these results by using isothermal titration calorimetry (ITC) (FIG. 1b) and found binding of EHD2 to the non-hydrolysable ATP analogue ATP-γ-S, with an affinity of 13 μM, and to ADP, with an affinity of approximately 50 μM. A mutation in the phosphate binding (P-)loop, T72A, prevented binding to ATP-γ-S, similar to the effect of equivalent mutations on nucleotide binding to other GTPases. No binding signal was observed for GTP-γ-S (FIG. 1b and¹⁵). We still refer to this domain as a G-domain because the sequence and fold (see below) are clearly similar to other G-domains. We next investigated membrane-binding properties of EHD2 and found efficient binding to liposomes made from brain-derived lipids (Folch extract) and to 100% anionic phosphatidyl-serine (PS) liposomes (FIG. 1c). Using synthetic liposomes containing various different phosphatidyl-inositols (PI) (FIG. 1c) we observed best binding to liposomes containing PI(4,5)bisphosphate (PIP2).

### Example 2

### Effects on Biological Membranes

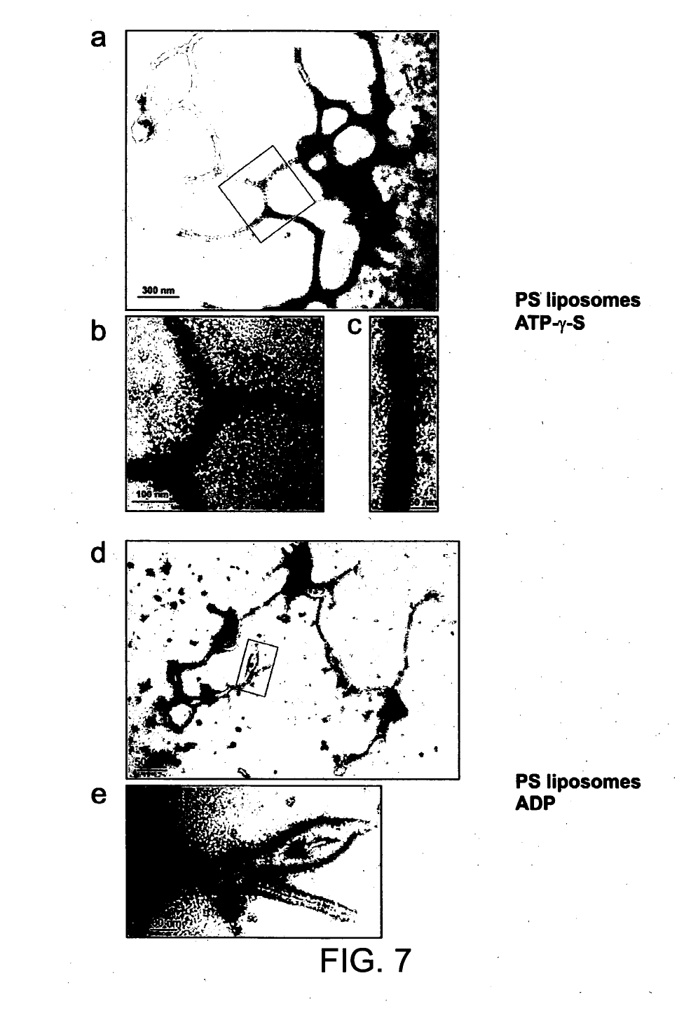

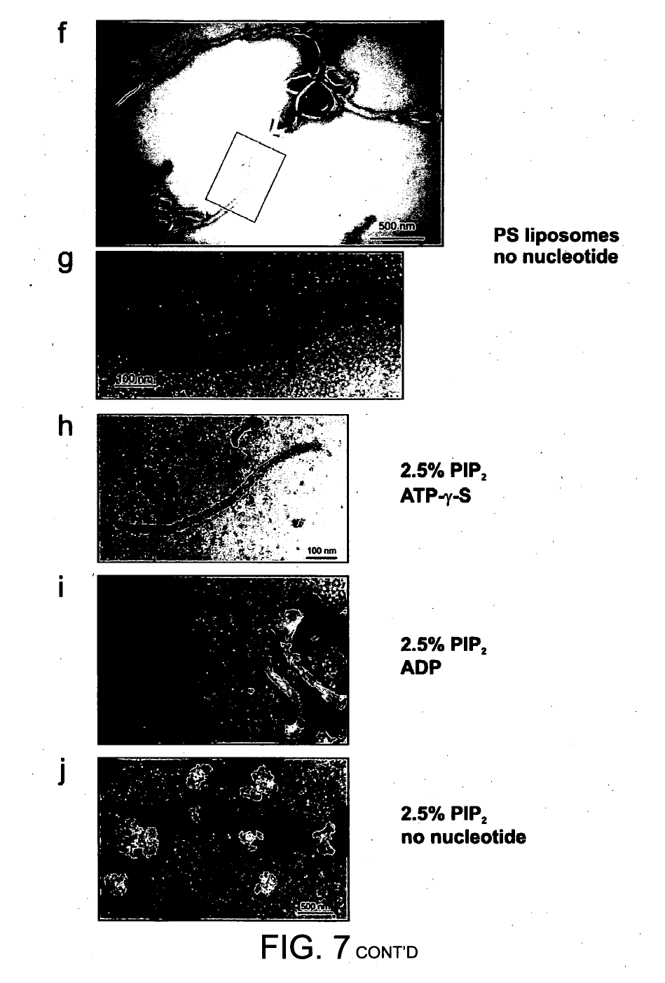

The consequence of membrane-binding was analysed by electron microscopy (EM), and we found that EHD2 deforms PS liposomes in a nucleotide-independent manner into 20 nm-diameter tubules and oligomerises in ring-like structures around these tubules (FIG. 1d, FIG. 7). Nucleotide-independence of liposome tubulation in vitro is also observed for dynamin¹⁶. No noticeable tubule fission or alteration in tubule diameter was found for EHD2 in the presence of ATP. Frequently we observed a complex network of connected tubules that had an extensive surface area implying that there is considerable fusion occurring between liposomes (FIG. 7). We also saw a few instances where EHD2 oligomeric rings were of variable diameter (FIG. 1d, bottom) suggesting that the interface used for EHD2 oligomerisation is rather flexible. Folch liposomes were also tabulated by EHD2. However, with synthetic liposomes containing only 2.5% PIP2 and in the absence of PS, the amount of EHD binding was reduced, and in this less favourable binding condition we only observed tubulation in the presence of ATP-γ-S and ADP but not in the absence of nucleotides (FIG. 7).

When EGFP-tagged EHD2 was over-expressed in HeLa cells it marked punctate and tubular structures that were mainly found close to the plasma membrane (FIG. 1e, endogenous EHD2 in various cell lines shows a similar peripheral distrubution^{13,17}). Although the nucleotide-free T72A mutant bound to Folch liposomes in vitro (FIG. 1C), it showed a cytoplasmic distribution when over-expressed in vivo (FIG. 1e), indicating that nucleotide binding is required for oligomerisation in vivo, in agreement with previous results³.

### Example 3

### ATP Binding and ATPase Activity

We next monitored the effect of membrane-binding on the ATPase activity of. EHD2 under multiple-turnover conditions (10-fold excess of ATP over EHD2), in the presence and absence of Folch liposoines (FIG. 1f). The intrinsic/background ATPase activity is extremely slow (kobs=0.7 h⁻¹) but is stimulated 8-fold in the presence of Folch or PS liposomes (kobs=5.6 h⁻¹). In contrast to GBP1¹⁸ we did not observe hydrolysis to nucleoside mono-phosphate. GTP was not hydrolysed in the presence or absence of Folch liposomes, and the T72A mutant did not show membrane-stimulated ATPase activity. EHD2 displays a 600-fold slower stimulated nucleotide hydrolysis than dynamin, which hydrolyses GTP under this condition with a kcat of ≈1 sec⁻¹.

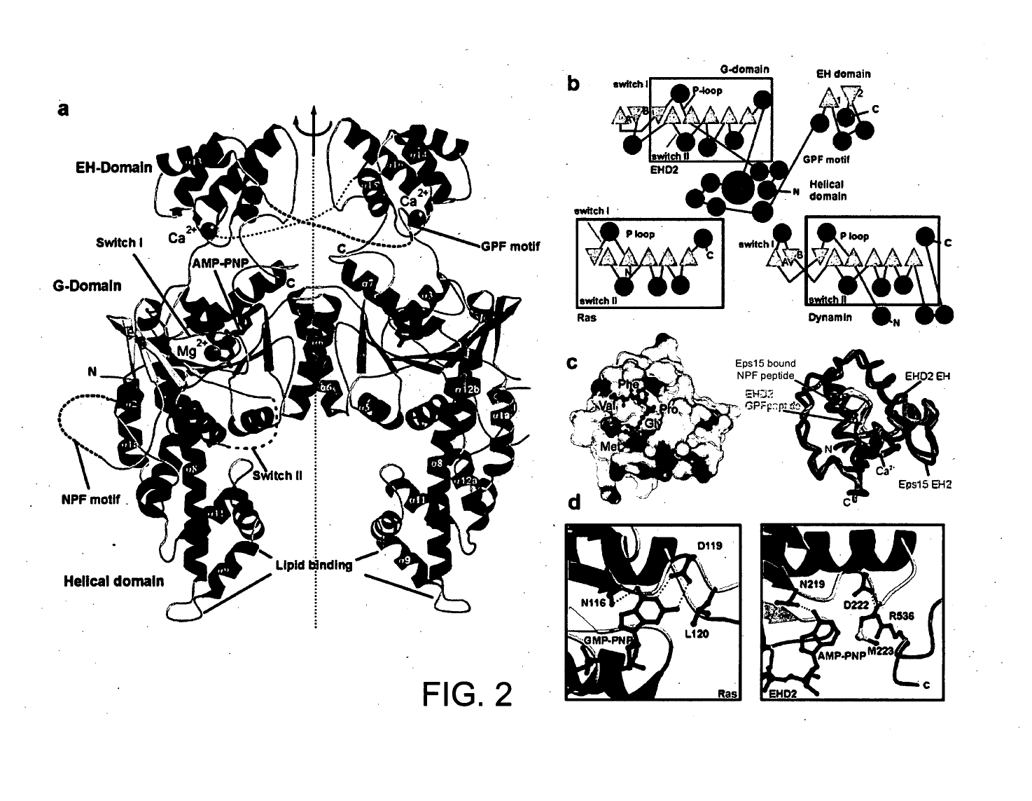

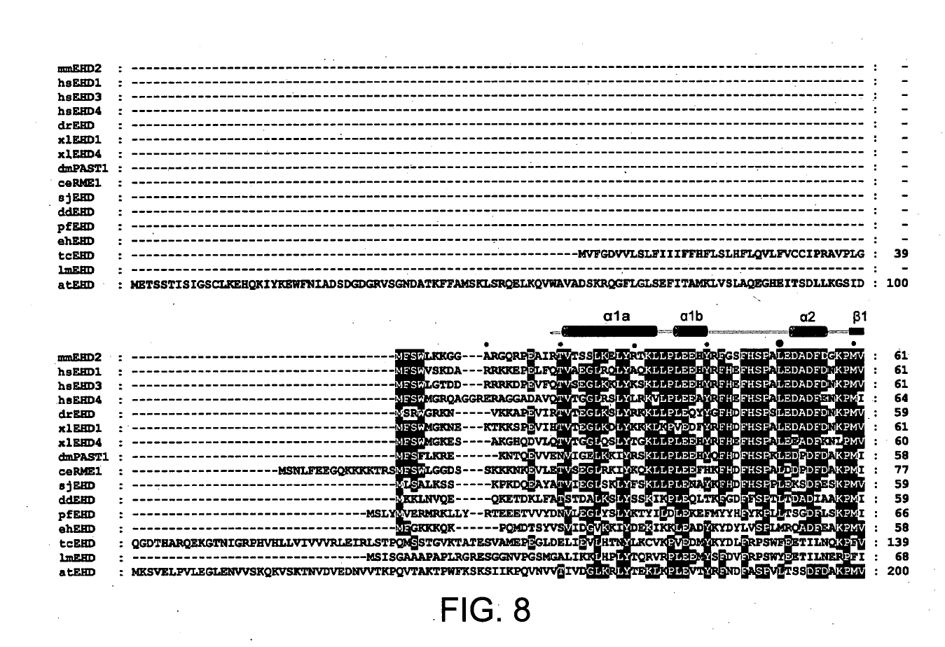

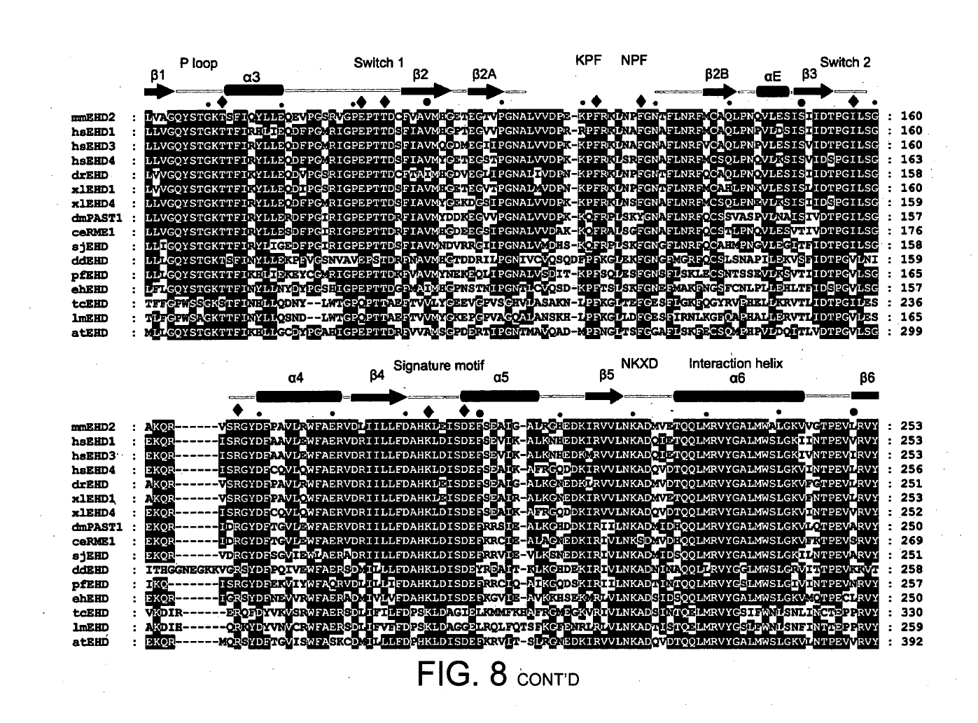

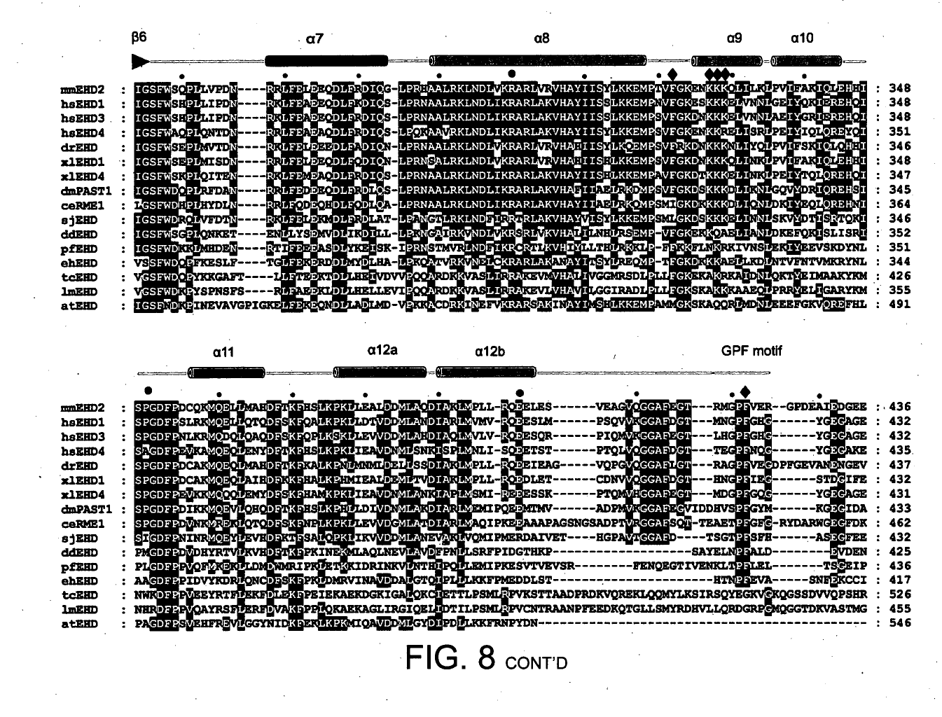

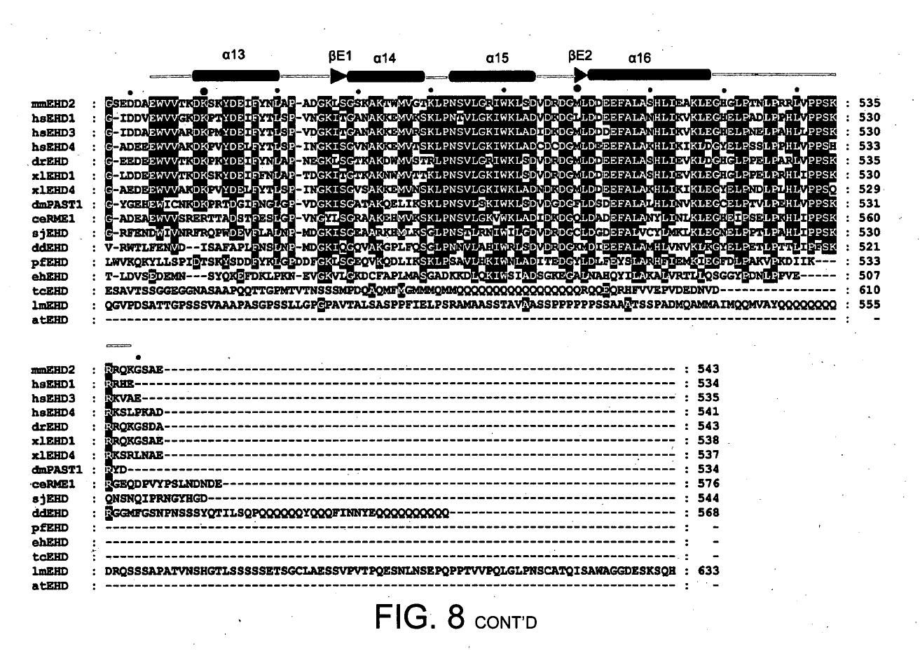

To obtain structural insights we solved the crystal structure of EHD2 in the presence of the non-hydrolysable ATP analogue AMP-PNP to a maximal resolution of 3.1 Å (see Methods and FIG. 15 (“Supplementary Table 1”) for the statistics). The nucleotide binding domain of EHD2 possesses a typical G-domain fold with a central β-sheet surrounded by α-helices (FIG. 2a, 2b and FIG. 8). An

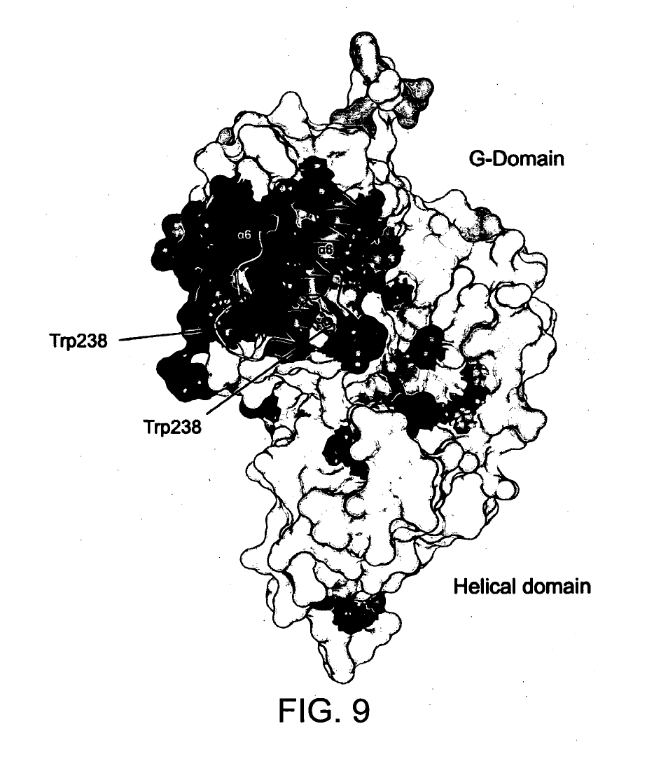

AMP-PNP molecule occupies the canonical nucleotide-binding site. In comparison to the Ras-like G-domain, EHD2 contains an insertion of two additional β-strands in the switch I region which are also present in the G-domain of dynamin¹⁹ (FIG. 2b). Residues 112-129, which are distal to switch I, are disordered and contain a predicted EH-domain binding motif, KPFxxxNPF. In agreement with our biochemical analysis, EHD2 crystallises as a dimer, and the dimer axis corresponds to a crystallographic two-fold axis (FIG. 2a). Dimerisation is mediated via a highly conserved, mostly hydrophobic interface of approximately 2100 Å² in the G-domain (FIG. 9). At the centre of the interface, the entirely conserved W238 in helix α6 is buried in a hydrophobic pocket, and mutations of this residue render the protein insoluble. The high conservation of this interface suggests that it is a common feature of all EHD members. Such an interface has not been described as yet for any other large GTPase and involves a different face of the G-domain than the dimer interfaces from the structurally characterised GBP1 and bacterial dynamin-like protein (BDLP) dimers^{18,20}.

### Example 4

### Structural Insights

The helical domain is composed of helix α1 and α2 from the N-terminal region (residues 18-55, which follow disordered residues 1-18) and helices α8, α9, α10, α11 and α12 (residues 285-400) following the G-domain (FIG. 2a). Helix α8 of EHD is the organising scaffold against which most of the other helices fold. It has also extensive contacts with the G-domain. The dimeric G-domain together with the helical region adopts a scissor shape, where the membrane is proposed to bind between blades (see later).

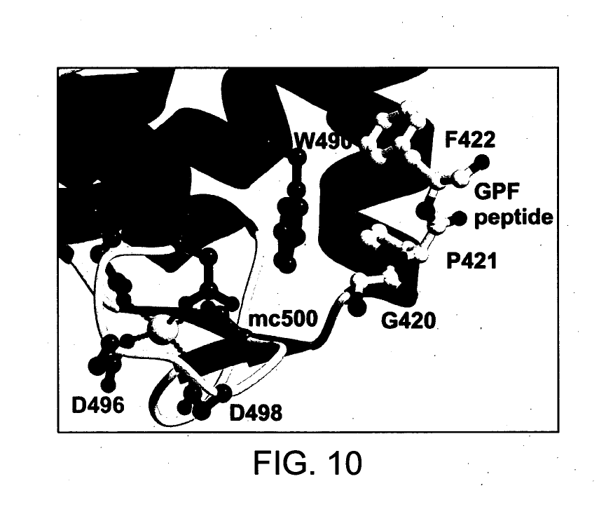

Following the middle domain there is a 40-residue linker which connects the helical domain with the C-terminal EH-domain (residues 443-543). The EH-domain of EHD2 is similar to the previously determined second EH-domain of Eps15 solved by NMR studies^{21,22} with a root-mean square deviation of 1.5 Å for the main-chain atoms (FIG. 2c). It is built of two closely packed perpendicular EF hands which are connected by a short β-sheet. We included a Ca²⁺-ion in the second EF hand which is ligated by four oxygens from acidic side-chains (D494, D496, D498, E505) and one main chain carbonyl oxygen of M500 (FIG. 10). This ion has been maintained during purification. The EH-domain is localised on top of the G-domain (the top site position) with a buried interface of 1600 Å². Eighteen disordered residues connecting the EHdomain to the helical domain means that it is ambiguous as to which EH-domain connects to which helical domain. We assign the EH-domains to opposing monomers (red EHD belongs to red helical domain in FIG. 2a) as the last visible residue of the linker from the helical domain is closer to the opposite EH-domain (distance 29 Å) than to the superjacent EH-domain (distance of 34 Å), and in this latter case the linker would have to wind around the EH-domain and thus one would expect to see some parts of this in the structure (FIG. 2a). Unexpectedly, the peptide binding sites of both EH-domains are occupied by a GPF motif (residues 420-422) from the linker region (FIGS. 2a and c). The GPF motif adopts a similar conformation as an NPF-containing peptide bound to the EH2 domain of Eps15²¹ involving a tight turn with F422 projecting into a hydrophobic pocket which is lined by W490 at its base (FIG. 2c and FIG. 10).

### Example 5

### Nucleiotide Binding Site

Specific guanosine recognition in GTPases is mediated by a highly conserved NKxD motif (called G4) where the asparagine side-chain forms a hydrogen bond to the carbonyl group at carbon6 of the guanosine base and the aspartate side-chain forms a double hydrogen bond to nitrogen1 of the guanosine base and the amine group at carbon2 (FIG. 2d, left). Aspartate has been shown to be crucial since mutating it to asparagine in Ras reduces nucleotide affinity by more than 1000-fold²³. Directly following the NKxD motif, a large hydrophobic residue (leucine in Ras and dynamin, M223 in EHD2) lines the nucleotide binding pocket. The NKxD motif of EHD2 (starting at residue 219) is also highly conserved in EHD family members (FIG. 8). The carboxyamide group of N219 forms a hydrogen bond to the C6 amino group of the adenine base (FIG. 2d, right). M223, whose side-chain is buttressed by the side chain of H192, is closer to the purine base than the corresponding leucine residue in Ras and sterically excludes an amino group at C2, thus explaining the inability of EHDs to bind to guanine nucleotides. Instead of forming a double hydrogen bond, the completely invariant D222 of EHD2 surprisingly forms a salt bridge with the conserved R536 which is supplied from the C-terminus of the superjacent EH-domain (FIG. 2d, right).

### Example 6

### Membrane Binding Site

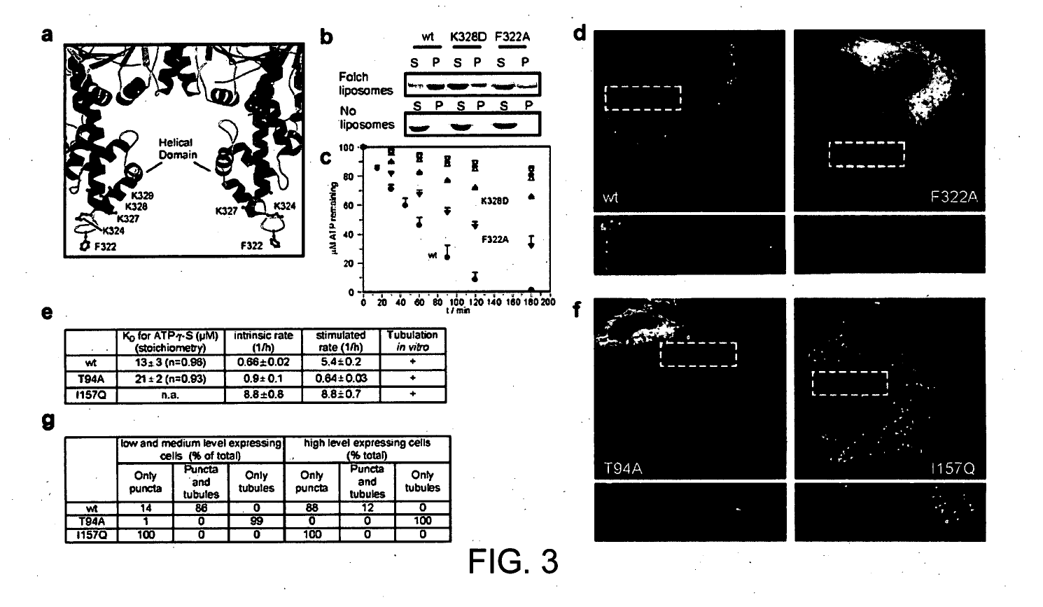

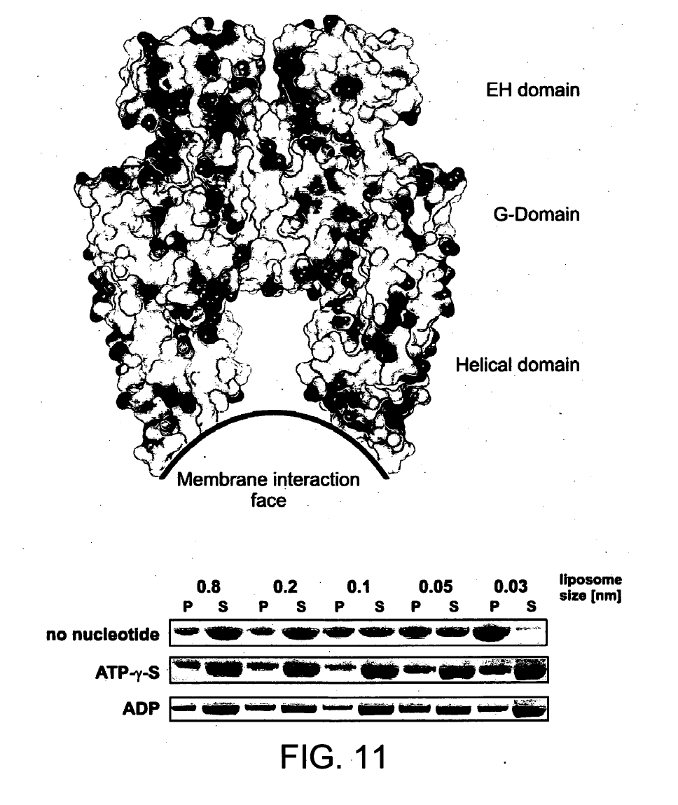

The membrane-binding properties of EHD2 are reminiscent of a subset of the small GTPase family which have recently been shown to require polybasic stretches for their PIP2- and PI(3,4,5)P3-dependent targeting to the plasma membrane²⁴. We found such a polybasic stretch in EHD2 close to the tip of the helical domain, but facing the cavity between the EHD2 dimer, consisting of K324, K327, K328 and K329 on each monomer (FIG. 3a). A hydrophobic residue, F322, is located at the tip of the helical domain. We mutated K328 to aspartate and F322 to alanine, and indeed both these mutants bound less to liposomes (FIG. 3b), and showed reduced ATPase stimulation in the presence of Folch liposomes (FIG. 3c). In vivo, the F322A mutation of the membrane-binding site, and in fact any lysine to aspartate mutations in the polybasic cluster, led to a completely cytoplasmic distribution of the protein (FIG. 3d). Thus the lipid interaction site of EHD2 is composed of two closely opposing lipid-binding sites in the dimer, and this leads to a highly curved membrane interface of approximately 7 nm radius (FIG. 3a and FIG. 11). Indeed EHD2 showed a binding preference for very small liposomes, consistent with this curvature, but only in the nucleotide-free form (FIG. 11). In the nucleotide-bound form EHD binding is not curvature sensitive, and this is likely due to oligomer formation along an axis perpendicular to the high curvature (see below).

### Example 7

### Catalytic Site

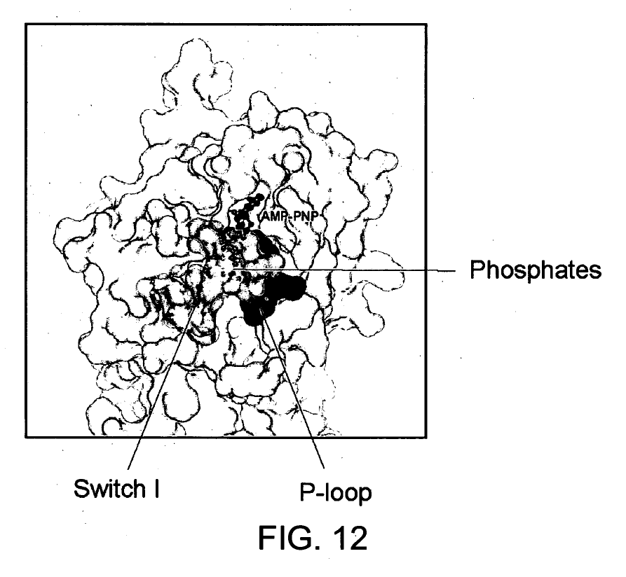

In EHD2, the phosphate groups of the AMP-PNP molecule are occluded from the exterior by switch I and the P-loop, which would not allow the insertion of a catalytic residue in trans into the catalytic site (FIG. 12). This so-called “phosphate cap” is also present in GBP 1²⁵ whose mechanism of GTP hydrolysis has been shown to involve dimerisation-dependent positioning of the attacking nucleophilic water molecule by a catalytic serine in cis from switch I¹⁸ which corresponds to T94 in EHD2.

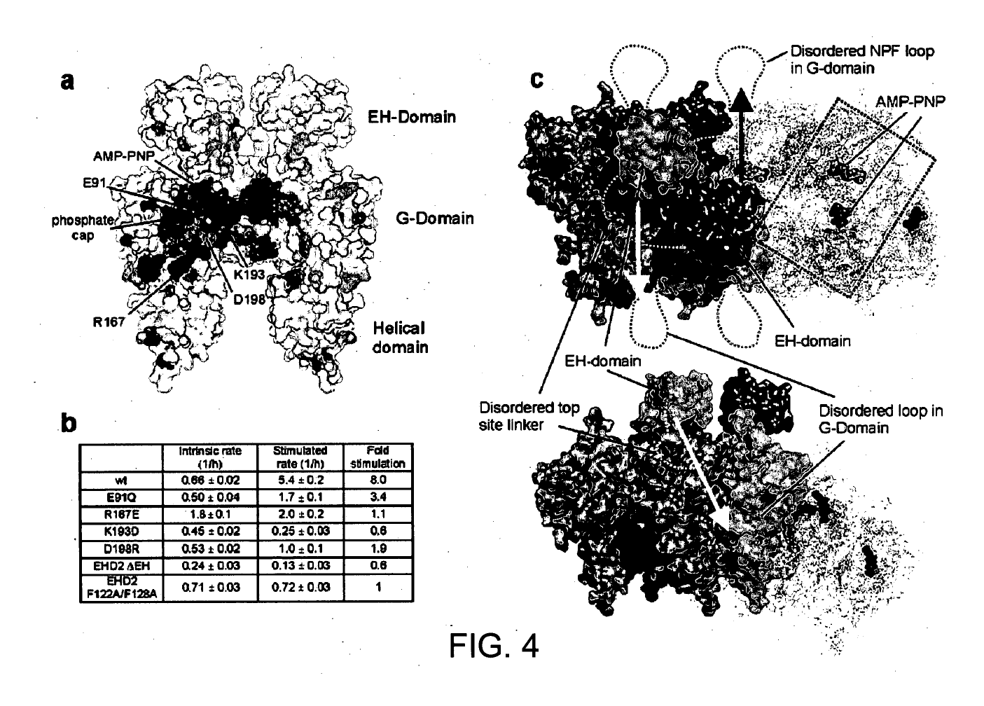

In small GTPases, the role of positioning the water molecule is assigned to a catalytic glutamine from switch II²⁶. We studied the role of ATP hydrolysis to gain insights into the function of EHD2 in vivo. The T94A mutant bound to ATP-γ-S with nearly wild-type affinity (FIG. 3e) and oligomerised around PS liposomes (FIG. 4e), but did not show any membrane-stimulated ATPase activity, consistent with a catalytic function for T94. When over-expressed in HeLa cells, the T94A mutant labelled extensive tubular structures with essentially, no punctate staining (compare wild-type and T94A in FIGS. 3d, 3f and 3g), suggesting that ATP hydrolysis is involved in the break-down of tubular structures in vivo. We previously observed severely inhibited GTP hydrolysis and a similar extensive tubulation phenotype with dynamin1 when the equivalent T65 was mutated to alanine⁷. To confirm the role of ATP hydrolysis in EHD function we re-introduced a catalytic glutamine in the active site, at the position normally found for small GTPases (I157Q). This mutant tubulated liposomes and hydrolysed ATP faster than wild-type, even in the absence of membranes. Furthermore, the ATPase reaction was not further stimulated by membranes (FIG. 3e). When over-expressed in HeLa cells this protein labelled only very short tubules and puncta (FIG. 3f, FIG. 3g). Many of these puncta were highly mobile as determined by live-cell microscopy. This suggests that increased ATP hydrolysis leads to increased membrane fission by EHD2. Altogether, these results are consistent with a role of ATP hydrolysis in the scission of membranes in vivo.

In a surface-conservation plot of the EHD2 dimer, a highly conserved surface patch encompasses switch I, switch II and the surrounding area (FIG. 4a). GBP1 and BDLP use this same interface, with the same relative orientation of the G-domains, for dimerisation^{18,20}. This interface was suggested to be a conserved feature of all dynaminrelated GTPases²⁰. Thus, the EHD2 dimer may further oligomerise into the observed rings using this second G-domain interface. Using the dimeric GBP1.GDP.AlF 4 G-domain structure¹⁸ as a template and taking into account the observed 20-nm ring of the EHD2 coat (FIG. 1c), we predicted the arrangement of the EHD2 dimers within the oligomer in which the nucleotides of two opposing EHD monomers are facing each other in a head-to-head fashion (see Methods and FIG. 4c). The predicted oligomer has a compact structure with a high degree of shape-complementarity between the oligomerising dimers (see Supplementary coordinates). Furthermore, the membrane-binding sites are pointing in the same direction towards the putative membrane interface (FIG. 4c, FIG. 13), and the thickness of the oligomeric ring agrees well with the thickness of the rings observed by EM (≈10 nm, FIG. 1d). We tested this model by mutagenesis of four surface-exposed conserved residues in this putative interface (FIG. 4a, 4b). As predicted, all mutants showed reduced or completely abolished assembly-stimulated ATP hydrolysis (FIG. 4b) indicating that the conserved interface is indeed involved in the assembly-stimulated ATPase reaction. In the predicted oligomer, the highly curved membrane interface of the EHD2 dimer are oriented perpendicular to the direction of the tubule curvature (FIG. 13b), consistent with the tightly packed rings observed by EM. In an alternative model, the oligomer could also form along the length of a tubule (parallel to its long axis) and thus maximise the use of the highly curved membrane interface of the dimer (FIG. 13c). However, such an arrangement was never observed by EM. Furthermore, our ring-based oligomer model is consistent with the absence of any curvature-sensitive membrane binding upon oligomerisation.

### Example 8

### EH Domains

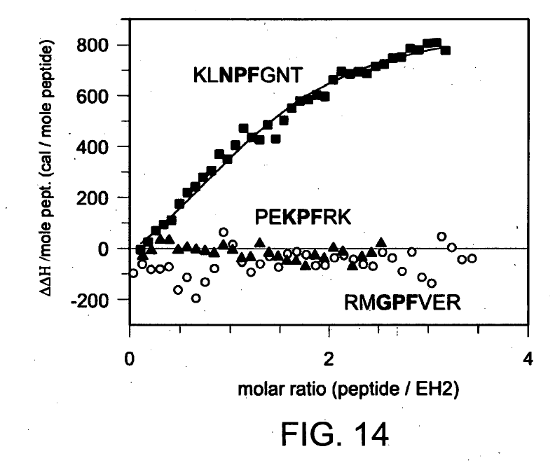

EH-domains are low affinity protein-protein interactors normally found in endocytic multi-subunit assemblies. A likely purpose of the EH-domain is in recruitment of the protein to sites of action. In the oligomer it could further function to concentrate NPF-containing binding partners around membrane-bound EHD, but we did not observe any co-localisation of described EHD binding partners, pacsin1 and 2²⁷ along the length of EHD tubules in vivo (data not shown). An alternative explanation for the function of the EH-domain in the oligomer can be proposed based on the presence of two conserved PF motifs (NPF and IPF) in a disordered surface loop at the side of the G-domain between β-sheet 2A and 2B. In our proposed oligomer, the EH-domains from one dimer are close to the side-site NPF motif of the adjacent dimer (FIG. 4c) and thus after oligomerisation on membranes they may switch position from the top site position in the dimer to the side site in the opposing dimer. It is interesting that only a side site NPF peptide, and not the top site GPF peptide, bound with a measurable affinity (≈130 μM) to the isolated EH-domain of EHD2 (FIG. 14), and this affinity is well within the range of other related EH-domain interactions²¹. In agreement with a role for the NPF motifs in stabilisation/organisation of the EHD oligomer we observed that a deletion mutant of the EH-domain (ΔEH) or a double mutant of the two side-site xPF motifs (F122A/F128A) did not show any membrane-stimulated ATPase activity (FIG. 4b) and we did not find any regular oligomers on liposomes in EM studies for these mutants. A dominant negative mutant in C. elegans Rme1 is found in the linker between the EH-domain and the helical domain³ pointing to the importance of this flexible linker.

### Example 9

### Cell Imaging Studies

EHD2 wild-type was over-expressed in HeLa cells for 24 h and imaged by EPI-fluorescence for approximately 30 min. Some of the tubules and puncta are dynamic.

EHD2 T94A was over-expressed in HeLa cells for 24 h and imaged by EPI-fluorescence for approximately 30 min. There a only tubules, and these are mostly static. EHD2 I157Q was over-expressed in HeLa cells for 24 h and imaged by EPI-fluorescence for approximately 30 min. No tubules can be found and the puncta are mostly motile.

Supplementary coordinates: Pdb coordinates of the proposed EHD2 oligomer. Four EHD2 dimers (in the absence of the EH domain) were aligned as described in Methods. All lipid interaction sites point towards the putative membrane interface. Molecules B and C which have been used for the initial alignment with GBP1 are related via a 2-fold axis, and the nucleotides of these molecules are oriented in a head-to-head fashion.

### Example 10

### Assay

This example relates to a method of identifying a modulator of an EHD family polypeptide.

A first and second sample of an EHD polypeptide are provided. In this example the EHD polypeptide is EHD2.

The first EHD sample is contacted with a candidate modulator. The candidate modulator is added to the medium containing the EHD polypeptide.

ATP reagent is added to the first and second samples. The ATP reagent is as per InnovaBiosciences' catalogue number 601-0120, permitting colorimetric readout of ATP hydrolysis.

ATP hydrolysis in said first and second samples is monitored in accordance with the manufacturer's instructions.

A difference between the ATP hydrolysis in said first and second samples identifies said candidate modulator as a modulator of an EHD family polypeptide.

In particular, if hydrolysis of ATP is greater in said first sample than in said second sample then the candidate modulator is identified as an enhancer of EHD family polypeptide activity. Conversely, if hydrolysis of ATP is lower in said, first sample than in said second sample then the candidate modulator is identified as an inhibitor of EHD family polypeptide activity.

The ATP hydrolysis is optionally monitored in the presence of lipid, in which case liposomes and/or phosphatidylserine (PS) at a final concentration of about 10% are added to both the samples, suitably after addition of candidate modulator but before addition of ATP reagent.

### Example 11

### Atomic Coordinates

TABLE A

| | | | | | | | | | | | |
|---|---|---|---|---|---|---|---|---|---|---|---|
| ATOM | 1 | N | ARG | A | 19 | 51.364 | −44.695 | −13.521 | 1.00 | 58.79 | N |
| ATOM | 2 | CA | ARG | A | 19 | 51.849 | −44.622 | −14.929 | 1.00 | 58.86 | C |
| ATOM | 3 | CB | ARG | A | 19 | 53.209 | −45.314 | −15.064 | 1.00 | 58.86 | C |
| ATOM | 4 | CG | ARG | A | 19 | 53.148 | −46.838 | −15.011 | 1.00 | 59.05 | C |
| ATOM | 5 | CD | ARG | A | 19 | 54.535 | −47.464 | −15.123 | 1.00 | 59.14 | C |
| ATOM | 6 | NE | ARG | A | 19 | 55.299 | −46.949 | −16.261 | 1.00 | 59.75 | N |
| ATOM | 7 | CZ | ARG | A | 19 | 56.300 | −46.076 | −16.161 | 1.00 | 59.95 | C |
| ATOM | 8 | NH1 | ARG | A | 19 | 56.669 | −45.613 | −14.972 | 1.00 | 60.07 | N |
| ATOM | 9 | NH2 | ARG | A | 19 | 56.937 | −45.665 | −17.252 | 1.00 | 59.86 | N |
| ATOM | 10 | C | ARG | A | 19 | 50.835 | −45.249 | −15.884 | 1.00 | 58.73 | C |
| ATOM | 11 | O | ARG | A | 19 | 51.082 | −45.372 | −17.083 | 1.00 | 58.69 | O |
| ATOM | 12 | N | THR | A | 20 | 49.688 | −45.636 | −15.338 | 1.00 | 58.62 | N |
| ATOM | 13 | CA | THR | A | 20 | 48.614 | −46.223 | −16.132 | 1.00 | 58.46 | C |
| ATOM | 14 | CB | THR | A | 20 | 47.790 | −47.226 | −15.302 | 1.00 | 58.50 | C |
| ATOM | 15 | OG1 | THR | A | 20 | 47.104 | −46.534 | −14.249 | 1.00 | 58.37 | O |
| ATOM | 16 | CG2 | THR | A | 20 | 48.699 | −48.292 | −14.705 | 1.00 | 58.59 | C |
| ATOM | 17 | C | THR | A | 20 | 47.680 | −45.152 | −16.697 | 1.00 | 58.32 | C |
| ATOM | 18 | O | THR | A | 20 | 48.020 | −43.965 | −16.718 | 1.00 | 58.25 | O |
| ATOM | 19 | N | VAL | A | 21 | 46.500 | −45.579 | −17.143 | 1.00 | 58.12 | N |
| ATOM | 20 | CA | VAL | A | 21 | 45.548 | −44.683 | −17.800 | 1.00 | 57.89 | C |
| ATOM | 21 | CB | VAL | A | 21 | 44.611 | −45.447 | −18.756 | 1.00 | 57.93 | C |
| ATOM | 22 | CG1 | VAL | A | 21 | 43.624 | −46.293 | −17.968 | 1.00 | 58.17 | C |
| ATOM | 23 | CG2 | VAL | A | 21 | 43.872 | −44.474 | −19.663 | 1.00 | 57.98 | C |
| ATOM | 24 | C | VAL | A | 21 | 44.712 | −43.859 | −16.817 | 1.00 | 57.68 | C |
| ATOM | 25 | O | VAL | A | 21 | 44.574 | −42.644 | −16.983 | 1.00 | 57.39 | O |
| ATOM | 26 | N | THR | A | 22 | 44.152 | −44.511 | −15.801 | 1.00 | 57.46 | N |
| ATOM | 27 | CA | THR | A | 22 | 43.359 | −43.792 | −14.806 | 1.00 | 57.30 | C |
| ATOM | 28 | CB | THR | A | 22 | 42.661 | −44.740 | −13.793 | 1.00 | 57.27 | C |
| ATOM | 29 | OG1 | THR | A | 22 | 43.523 | −44.987 | −12.676 | 1.00 | 57.51 | O |
| ATOM | 30 | CG2 | THR | A | 22 | 42.280 | −46.062 | −14.453 | 1.00 | 57.23 | C |
| ATOM | 31 | C | THR | A | 22 | 44.255 | −42.784 | −14.085 | 1.00 | 57.11 | C |
| ATOM | 32 | O | THR | A | 22 | 43.837 | −41.669 | −13.783 | 1.00 | 57.00 | O |
| ATOM | 33 | N | SER | A | 23 | 45.494 | −43.183 | −13.824 | 1.00 | 56.93 | N |
| ATOM | 34 | CA | SER | A | 23 | 46.500 | −42.275 | −13.297 | 1.00 | 56.79 | C |
| ATOM | 35 | CB | SER | A | 23 | 47.849 | −42.985 | −13.265 | 1.00 | 56.78 | C |
| ATOM | 36 | OG | SER | A | 23 | 48.811 | −42.263 | −14.009 | 1.00 | 57.00 | O |
| ATOM | 37 | C | SER | A | 23 | 46.594 | −41.016 | −14.155 | 1.00 | 56.73 | C |
| ATOM | 38 | O | SER | A | 23 | 46.504 | −39.884 | −13.653 | 1.00 | 56.72 | O |
| ATOM | 39 | N | SER | A | 24 | 46.768 | −41.227 | −15.456 | 1.00 | 56.67 | N |
| ATOM | 40 | CA | SER | A | 24 | 46.884 | −40.135 | −16.415 | 1.00 | 56.66 | C |
| ATOM | 41 | CB | SER | A | 24 | 47.130 | −40.691 | −17.818 | 1.00 | 56.64 | C |
| ATOM | 42 | OG | SER | A | 24 | 47.818 | −39.753 | −18.626 | 1.00 | 56.92 | O |
| ATOM | 43 | C | SER | A | 24 | 45.629 | −39.262 | −16.388 | 1.00 | 56.55 | C |
| ATOM | 44 | O | SER | A | 24 | 45.710 | −38.031 | −16.372 | 1.00 | 56.41 | O |
| ATOM | 45 | N | LEU | A | 25 | 44.471 | −39.911 | −16.373 | 1.00 | 56.64 | N |
| ATOM | 46 | CA | LEU | A | 25 | 43.208 | −39.213 | −16.174 | 1.00 | 56.46 | C |
| ATOM | 47 | CB | LEU | A | 25 | 42.067 | −40.216 | −16.016 | 1.00 | 56.37 | C |
| ATOM | 48 | CG | LEU | A | 25 | 41.205 | −40.508 | −17.240 | 1.00 | 56.13 | C |
| ATOM | 49 | CD1 | LEU | A | 25 | 41.983 | −40.290 | −18.513 | 1.00 | 55.71 | C |
| ATOM | 50 | CD2 | LEU | A | 25 | 40.657 | −41.923 | −17.166 | 1.00 | 56.30 | C |
| ATOM | 51 | C | LEU | A | 25 | 43.271 | −38.320 | −14.940 | 1.00 | 56.49 | C |
| ATOM | 52 | O | LEU | A | 25 | 42.889 | −37.148 | −14.989 | 1.00 | 56.42 | O |
| ATOM | 53 | N | LYS | A | 26 | 43.752 | −38.883 | −13.835 | 1.00 | 56.45 | N |
| ATOM | 54 | CA | LYS | A | 26 | 43.817 | −38.164 | −12.575 | 1.00 | 56.29 | C |
| ATOM | 55 | CB | LYS | A | 26 | 44.322 | −39.079 | −11.456 | 1.00 | 56.32 | C |
| ATOM | 56 | CG | LYS | A | 26 | 44.300 | −38.454 | −10.064 | 1.00 | 56.35 | C |
| ATOM | 57 | CD | LYS | A | 26 | 44.278 | −39.516 | −8.966 | 1.00 | 56.35 | C |
| ATOM | 58 | CE | LYS | A | 26 | 45.538 | −40.371 | −8.973 | 1.00 | 56.39 | C |
| ATOM | 59 | NZ | LYS | A | 26 | 45.407 | −41.557 | −8.080 | 1.00 | 56.13 | N |
| ATOM | 60 | C | LYS | A | 26 | 44.690 | −36.924 | −12.715 | 1.00 | 56.32 | C |
| ATOM | 61 | O | LYS | A | 26 | 44.264 | −35.817 | −12.367 | 1.00 | 56.34 | O |
| ATOM | 62 | N | GLU | A | 27 | 45.901 | −37.093 | −13.242 | 1.00 | 56.37 | N |
| ATOM | 63 | CA | GLU | A | 27 | 46.764 | −35.927 | −13.450 | 1.00 | 56.31 | C |
| ATOM | 64 | CB | GLU | A | 27 | 48.135 | −36.332 | −13.989 | 1.00 | 56.40 | C |
| ATOM | 65 | CG | GLU | A | 27 | 49.118 | −36.769 | −12.907 | 1.00 | 56.99 | C |
| ATOM | 66 | CD | GLU | A | 27 | 49.194 | −35.789 | −11.741 | 1.00 | 57.43 | C |
| ATOM | 67 | OE1 | GLU | A | 27 | 48.165 | −35.604 | −11.049 | 1.00 | 57.97 | O |
| ATOM | 68 | OE2 | GLU | A | 27 | 50.285 | −35.218 | −11.504 | 1.00 | 56.59 | O |
| ATOM | 69 | C | GLU | A | 27 | 46.110 | −34.886 | −14.357 | 1.00 | 56.16 | C |
| ATOM | 70 | O | GLU | A | 27 | 46.094 | −33.691 | −14.039 | 1.00 | 55.99 | O |
| ATOM | 71 | N | LEU | A | 28 | 45.563 | −35.352 | −15.478 | 1.00 | 56.19 | N |
| ATOM | 72 | CA | LEU | A | 28 | 44.827 | −34.483 | −16.394 | 1.00 | 56.04 | C |
| ATOM | 73 | CB | LEU | A | 28 | 44.156 | −35.295 | −17.505 | 1.00 | 55.97 | C |
| ATOM | 74 | CG | LEU | A | 28 | 44.864 | −35.366 | −18.861 | 1.00 | 56.13 | C |
| ATOM | 75 | CD1 | LEU | A | 28 | 46.354 | −35.712 | −18.746 | 1.00 | 56.25 | C |
| ATOM | 76 | CD2 | LEU | A | 28 | 44.144 | −36.321 | −19.799 | 1.00 | 56.01 | C |
| ATOM | 77 | C | LEU | A | 28 | 43.787 | −33.671 | −15.642 | 1.00 | 56.00 | C |
| ATOM | 78 | O | LEU | A | 28 | 43.736 | −32.450 | −15.767 | 1.00 | 56.01 | O |
| ATOM | 79 | N | TYR | A | 29 | 42.961 | −34.353 | −14.857 | 1.00 | 55.97 | N |
| ATOM | 80 | CA | TYR | A | 29 | 41.978 | −33.671 | −14.035 | 1.00 | 55.76 | C |
| ATOM | 81 | CB | TYR | A | 29 | 41.222 | −34.659 | −13.152 | 1.00 | 55.53 | C |
| ATOM | 82 | CG | TYR | A | 29 | 40.319 | −33.982 | −12.150 | 1.00 | 55.13 | C |
| ATOM | 83 | CD1 | TYR | A | 29 | 39.169 | −33.320 | −12.560 | 1.00 | 54.84 | C |
| ATOM | 84 | CE1 | TYR | A | 29 | 38.340 | −32.696 | −11.649 | 1.00 | 54.64 | C |
| ATOM | 85 | CZ | TYR | A | 29 | 38.657 | −32.729 | −10.309 | 1.00 | 54.85 | C |
| ATOM | 86 | OH | TYR | A | 29 | 37.839 | −32.111 | −9.396 | 1.00 | 55.02 | O |
| ATOM | 87 | CE2 | TYR | A | 29 | 39.791 | −33.381 | −9.875 | 1.00 | 54.94 | C |
| ATOM | 88 | CD2 | TYR | A | 29 | 40.617 | −34.000 | −10.796 | 1.00 | 54.98 | C |
| ATOM | 89 | C | TYR | A | 29 | 42.640 | −32.618 | −13.163 | 1.00 | 55.90 | C |
| ATOM | 90 | O | TYR | A | 29 | 42.328 | −31.432 | −13.272 | 1.00 | 55.94 | O |
| ATOM | 91 | N | ARG | A | 30 | 43.565 | −33.056 | −12.311 | 1.00 | 56.07 | N |
| ATOM | 92 | CA | ARG | A | 30 | 44.161 | −32.167 | −11.317 | 1.00 | 56.16 | C |
| ATOM | 93 | CB | ARG | A | 30 | 45.155 | −32.915 | −10.425 | 1.00 | 56.13 | C |
| ATOM | 94 | CG | ARG | A | 30 | 45.781 | −32.027 | −9.349 | 1.00 | 56.57 | C |
| ATOM | 95 | CD | ARG | A | 30 | 46.944 | −32.704 | −8.639 | 1.00 | 56.62 | C |
| ATOM | 96 | NE | ARG | A | 30 | 47.997 | −33.102 | −9.570 | 1.00 | 57.56 | N |
| ATOM | 97 | CZ | ARG | A | 30 | 49.025 | −32.334 | −9.915 | 1.00 | 57.96 | C |
| ATOM | 98 | NH1 | ARG | A | 30 | 49.150 | −31.115 | −9.404 | 1.00 | 58.09 | N |
| ATOM | 99 | NH2 | ARG | A | 30 | 49.933 | −32.785 | −10.771 | 1.00 | 58.17 | N |
| ATOM | 100 | C | ARG | A | 30 | 44.836 | −30.945 | −11.925 | 1.00 | 56.07 | C |
| ATOM | 101 | O | ARG | A | 30 | 44.809 | −29.864 | −11.339 | 1.00 | 56.10 | O |
| ATOM | 102 | N | THR | A | 31 | 45.438 | −31.104 | −13.097 | 1.00 | 56.02 | N |
| ATOM | 103 | CA | THR | A | 31 | 46.186 | −29.999 | −13.689 | 1.00 | 55.94 | C |
| ATOM | 104 | CB | THR | A | 31 | 47.468 | −30.495 | −14.373 | 1.00 | 55.94 | C |
| ATOM | 105 | OG1 | THR | A | 31 | 47.127 | −31.427 | −15.407 | 1.00 | 56.01 | O |
| ATOM | 106 | CG2 | THR | A | 31 | 48.379 | −31.172 | −13.361 | 1.00 | 55.91 | C |
| ATOM | 107 | C | THR | A | 31 | 45.378 | −29.166 | −14.687 | 1.00 | 55.97 | C |
| ATOM | 108 | O | THR | A | 31 | 45.658 | −27.982 | −14.883 | 1.00 | 55.91 | O |
| ATOM | 109 | N | LYS | A | 32 | 44.377 | −29.780 | −15.311 | 1.00 | 56.00 | N |
| ATOM | 110 | CA | LYS | A | 32 | 43.660 | −29.137 | −16.414 | 1.00 | 56.02 | C |
| ATOM | 111 | CB | LYS | A | 32 | 43.701 | −30.019 | −17.673 | 1.00 | 56.03 | C |
| ATOM | 112 | CG | LYS | A | 32 | 45.082 | −30.162 | −18.339 | 1.00 | 56.05 | C |
| ATOM | 113 | CD | LYS | A | 32 | 45.420 | −28.956 | −19.218 | 1.00 | 56.16 | C |
| ATOM | 114 | CE | LYS | A | 32 | 46.772 | −29.099 | −19.924 | 1.00 | 55.96 | C |
| ATOM | 115 | NZ | LYS | A | 32 | 46.693 | −29.805 | −21.237 | 1.00 | 55.50 | N |
| ATOM | 116 | C | LYS | A | 32 | 42.211 | −28.750 | −16.087 | 1.00 | 56.03 | C |
| ATOM | 117 | O | LYS | A | 32 | 41.668 | −27.819 | −16.683 | 1.00 | 56.12 | O |
| ATOM | 118 | N | LEU | A | 33 | 41.587 | −29.460 | −15.149 | 1.00 | 55.98 | N |
| ATOM | 119 | CA | LEU | A | 33 | 40.182 | −29.209 | −14.814 | 1.00 | 55.83 | C |
| ATOM | 120 | CB | LEU | A | 33 | 39.381 | −30.508 | −14.852 | 1.00 | 55.81 | C |
| ATOM | 121 | CG | LEU | A | 33 | 38.353 | −30.636 | −15.968 | 1.00 | 55.96 | C |
| ATOM | 122 | CD1 | LEU | A | 33 | 37.675 | −31.991 | −15.893 | 1.00 | 56.34 | C |
| ATOM | 123 | CD2 | LEU | A | 33 | 37.330 | −29.526 | −15.847 | 1.00 | 56.25 | C |
| ATOM | 124 | C | LEU | A | 33 | 39.996 | −28.553 | −13.455 | 1.00 | 55.75 | C |
| ATOM | 125 | O | LEU | A | 33 | 39.334 | −27.521 | −13.332 | 1.00 | 55.70 | O |
| ATOM | 126 | N | LEU | A | 34 | 40.581 | −29.171 | −12.435 | 1.00 | 55.70 | N |
| ATOM | 127 | CA | LEU | A | 34 | 40.452 | −28.699 | −11.066 | 1.00 | 55.64 | C |
| ATOM | 128 | CB | LEU | A | 34 | 41.427 | −29.447 | −10.154 | 1.00 | 55.58 | C |
| ATOM | 129 | CG | LEU | A | 34 | 41.327 | −29.178 | −8.654 | 1.00 | 55.53 | C |
| ATOM | 130 | CD1 | LEU | A | 34 | 39.877 | −29.201 | −8.195 | 1.00 | 55.57 | C |
| ATOM | 131 | CD2 | LEU | A | 34 | 42.156 | −30.193 | −7.882 | 1.00 | 55.60 | C |
| ATOM | 132 | C | LEU | A | 34 | 40.638 | −27.189 | −10.935 | 1.00 | 55.74 | C |
| ATOM | 133 | O | LEU | A | 34 | 39.868 | −26.536 | −10.231 | 1.00 | 55.74 | O |
| ATOM | 134 | N | PRO | A | 35 | 41.658 | −26.624 | −11.611 | 1.00 | 55.81 | N |
| ATOM | 135 | CA | PRO | A | 35 | 41.887 | −25.187 | −11.486 | 1.00 | 55.89 | C |
| ATOM | 136 | CB | PRO | A | 35 | 43.070 | −24.935 | −12.426 | 1.00 | 55.71 | C |
| ATOM | 137 | CG | PRO | A | 35 | 43.742 | −26.244 | −12.550 | 1.00 | 55.71 | C |
| ATOM | 138 | CD | PRO | A | 35 | 42.647 | −27.256 | −12.502 | 1.00 | 55.76 | C |
| ATOM | 139 | C | PRO | A | 35 | 40.683 | −24.372 | −11.935 | 1.00 | 56.06 | C |
| ATOM | 140 | O | PRO | A | 35 | 40.384 | −23.339 | −11.341 | 1.00 | 56.11 | O |
| ATOM | 141 | N | LEU | A | 36 | 39.996 | −24.843 | −12.969 | 1.00 | 56.32 | N |
| ATOM | 142 | CA | LEU | A | 36 | 38.876 | −24.111 | −13.545 | 1.00 | 56.60 | C |
| ATOM | 143 | CB | LEU | A | 36 | 38.587 | −24.654 | −14.936 | 1.00 | 56.61 | C |
| ATOM | 144 | CG | LEU | A | 36 | 38.094 | −23.658 | −15.978 | 1.00 | 56.89 | C |
| ATOM | 145 | CD1 | LEU | A | 36 | 38.653 | −24.031 | −17.331 | 1.00 | 57.25 | C |
| ATOM | 146 | CD2 | LEU | A | 36 | 36.582 | −23.624 | −16.011 | 1.00 | 57.53 | C |
| ATOM | 147 | C | LEU | A | 36 | 37.648 | −24.229 | −12.646 | 1.00 | 56.79 | C |
| ATOM | 148 | O | LEU | A | 36 | 36.943 | −23.237 | −12.376 | 1.00 | 56.95 | O |
| ATOM | 149 | N | GLU | A | 37 | 37.413 | −25.448 | −12.171 | 1.00 | 57.00 | N |
| ATOM | 150 | CA | GLU | A | 37 | 36.379 | −25.705 | −11.180 | 1.00 | 57.34 | C |
| ATOM | 151 | CB | GLU | A | 37 | 36.412 | −27.168 | −10.732 | 1.00 | 57.32 | C |
| ATOM | 152 | CG | GLU | A | 37 | 36.068 | −28.182 | −11.809 | 1.00 | 57.49 | C |
| ATOM | 153 | CD | GLU | A | 37 | 36.009 | −29.602 | −11.266 | 1.00 | 57.48 | C |
| ATOM | 154 | OE1 | GLU | A | 37 | 35.934 | −29.763 | −10.026 | 1.00 | 57.98 | O |
| ATOM | 155 | OE2 | GLU | A | 37 | 36.035 | −30.557 | −12.073 | 1.00 | 57.15 | O |
| ATOM | 156 | C | GLU | A | 37 | 36.550 | −24.800 | −9.958 | 1.00 | 57.50 | C |
| ATOM | 157 | O | GLU | A | 37 | 35.595 | −24.154 | −9.517 | 1.00 | 57.53 | O |
| ATOM | 158 | N | GLU | A | 38 | 37.767 | −24.760 | −9.417 | 1.00 | 57.64 | N |
| ATOM | 159 | CA | GLU | A | 38 | 38.044 | −23.989 | −8.206 | 1.00 | 57.92 | C |
| ATOM | 160 | CB | GLU | A | 38 | 39.439 | −24.302 | −7.661 | 1.00 | 58.10 | C |
| ATOM | 161 | CG | GLU | A | 38 | 39.552 | −25.674 | −7.015 | 1.00 | 59.87 | C |
| ATOM | 162 | CD | GLU | A | 38 | 38.818 | −25.767 | −5.682 | 1.00 | 62.05 | C |
| ATOM | 163 | OE1 | GLU | A | 38 | 39.351 | −25.231 | −4.678 | 1.00 | 62.81 | O |
| ATOM | 164 | OE2 | GLU | A | 38 | 37.719 | −26.384 | −5.638 | 1.00 | 62.50 | O |
| ATOM | 165 | C | GLU | A | 38 | 37.901 | −22.498 | −8.441 | 1.00 | 57.70 | C |
| ATOM | 166 | O | GLU | A | 38 | 37.434 | −21.771 | −7.567 | 1.00 | 57.74 | O |
| ATOM | 167 | N | HIS | A | 39 | 38.298 | −22.045 | −9.624 | 1.00 | 57.50 | N |
| ATOM | 168 | CA | HIS | A | 39 | 38.240 | −20.627 | −9.932 | 1.00 | 57.36 | C |
| ATOM | 169 | CB | HIS | A | 39 | 39.053 | −20.289 | −11.186 | 1.00 | 57.49 | C |
| ATOM | 170 | CG | HIS | A | 39 | 39.414 | −18.837 | −11.301 | 1.00 | 57.86 | C |
| ATOM | 171 | ND1 | HIS | A | 39 | 39.302 | −18.130 | −12.480 | 1.00 | 58.15 | N |
| ATOM | 172 | CE1 | HIS | A | 39 | 39.687 | −16.882 | −12.283 | 1.00 | 57.80 | C |
| ATOM | 173 | NE2 | HIS | A | 39 | 40.040 | −16.751 | −11.017 | 1.00 | 58.12 | N |
| ATOM | 174 | CD2 | HIS | A | 39 | 39.879 | −17.959 | −10.381 | 1.00 | 57.91 | C |
| ATOM | 175 | C | HIS | A | 39 | 36.801 | −20.174 | −10.088 | 1.00 | 57.12 | C |
| ATOM | 176 | O | HIS | A | 39 | 36.415 | −19.146 | −9.542 | 1.00 | 57.14 | O |
| ATOM | 177 | N | TYR | A | 40 | 35.994 | −20.938 | −10.815 | 1.00 | 56.95 | N |
| ATOM | 178 | CA | TYR | A | 40 | 34.610 | −20.504 | −10.998 | 1.00 | 56.80 | C |
| ATOM | 179 | CB | TYR | A | 40 | 34.183 | −20.668 | −12.451 | 1.00 | 56.68 | C |
| ATOM | 180 | CG | TYR | A | 40 | 35.005 | −19.812 | −13.382 | 1.00 | 56.39 | C |
| ATOM | 181 | CD1 | TYR | A | 40 | 34.678 | −18.482 | −13.599 | 1.00 | 56.19 | C |
| ATOM | 182 | CE1 | TYR | A | 40 | 35.429 | −17.692 | −14.438 | 1.00 | 56.13 | C |
| ATOM | 183 | CZ | TYR | A | 40 | 36.532 | −18.227 | −15.072 | 1.00 | 56.27 | C |
| ATOM | 184 | OH | TYR | A | 40 | 37.288 | −17.435 | −15.910 | 1.00 | 56.35 | O |
| ATOM | 185 | CE2 | TYR | A | 40 | 36.884 | −19.546 | −14.866 | 1.00 | 55.86 | C |
| ATOM | 186 | CD2 | TYR | A | 40 | 36.124 | −20.327 | −14.025 | 1.00 | 56.00 | C |
| ATOM | 187 | C | TYR | A | 40 | 33.632 | −21.152 | −10.015 | 1.00 | 56.98 | C |
| ATOM | 188 | O | TYR | A | 40 | 32.416 | −21.131 | −10.219 | 1.00 | 56.80 | O |
| ATOM | 189 | N | ARG | A | 41 | 34.185 | −21.710 | −8.940 | 1.00 | 57.29 | N |
| ATOM | 190 | CA | ARG | A | 41 | 33.402 | −22.199 | −7.801 | 1.00 | 57.67 | C |
| ATOM | 191 | CB | ARG | A | 41 | 32.759 | −21.025 | −7.056 | 1.00 | 57.84 | C |
| ATOM | 192 | CG | ARG | A | 41 | 33.747 | −20.264 | −6.192 | 1.00 | 59.24 | C |
| ATOM | 193 | CD | ARG | A | 41 | 34.206 | −21.140 | −5.036 | 1.00 | 61.74 | C |
| ATOM | 194 | NE | ARG | A | 41 | 35.654 | −21.097 | −4.852 | 1.00 | 63.78 | N |
| ATOM | 195 | CZ | ARG | A | 41 | 36.369 | −22.080 | −4.309 | 1.00 | 65.07 | C |
| ATOM | 196 | NH1 | ARG | A | 41 | 37.684 | −21.953 | −4.184 | 1.00 | 65.94 | N |
| ATOM | 197 | NH2 | ARG | A | 41 | 35.775 | −23.194 | −3.895 | 1.00 | 65.68 | N |
| ATOM | 198 | C | ARG | A | 41 | 32.362 | −23.250 | −8.179 | 1.00 | 57.59 | C |
| ATOM | 199 | O | ARG | A | 41 | 31.245 | −23.266 | −7.656 | 1.00 | 57.63 | O |
| ATOM | 200 | N | PHE | A | 42 | 32.761 | −24.135 | −9.085 | 1.00 | 57.42 | N |
| ATOM | 201 | CA | PHE | A | 42 | 31.940 | −25.247 | −9.536 | 1.00 | 57.25 | C |
| ATOM | 202 | CB | PHE | A | 42 | 32.803 | −26.170 | −10.400 | 1.00 | 57.11 | C |
| ATOM | 203 | CG | PHE | A | 42 | 32.033 | −27.222 | −11.132 | 1.00 | 56.65 | C |
| ATOM | 204 | CD1 | PHE | A | 42 | 32.041 | −28.533 | −10.692 | 1.00 | 56.29 | C |
| ATOM | 205 | CE1 | PHE | A | 42 | 31.340 | −29.506 | −11.365 | 1.00 | 56.21 | C |
| ATOM | 206 | CZ | PHE | A | 42 | 30.623 | −29.177 | −12.498 | 1.00 | 56.63 | C |
| ATOM | 207 | CE2 | PHE | A | 42 | 30.611 | −27.873 | −12.952 | 1.00 | 56.58 | C |
| ATOM | 208 | CD2 | PHE | A | 42 | 31.315 | −26.905 | −12.271 | 1.00 | 56.58 | C |
| ATOM | 209 | C | PHE | A | 42 | 31.366 | −26.027 | −8.358 | 1.00 | 57.37 | C |
| ATOM | 210 | O | PHE | A | 42 | 30.240 | −26.520 | −8.414 | 1.00 | 57.37 | O |
| ATOM | 211 | N | GLY | A | 43 | 32.149 | −26.124 | −7.288 | 1.00 | 57.54 | N |
| ATOM | 212 | CA | GLY | A | 43 | 31.795 | −26.943 | −6.133 | 1.00 | 57.71 | C |
| ATOM | 213 | C | GLY | A | 43 | 30.592 | −26.473 | −5.338 | 1.00 | 57.88 | C |
| ATOM | 214 | O | GLY | A | 43 | 30.052 | −27.222 | −4.525 | 1.00 | 58.01 | O |
| ATOM | 215 | N | SER | A | 44 | 30.171 | −25.234 | −5.563 | 1.00 | 57.99 | N |
| ATOM | 216 | CA | SER | A | 44 | 29.037 | −24.680 | −4.838 | 1.00 | 58.04 | C |
| ATOM | 217 | CB | SER | A | 44 | 29.377 | −23.298 | −4.295 | 1.00 | 57.99 | C |
| ATOM | 218 | OG | SER | A | 44 | 30.592 | −23.328 | −3.568 | 1.00 | 58.35 | O |
| ATOM | 219 | C | SER | A | 44 | 27.824 | −24.589 | −5.739 | 1.00 | 58.15 | C |
| ATOM | 220 | O | SER | A | 44 | 26.838 | −23.930 | −5.401 | 1.00 | 58.23 | O |
| ATOM | 221 | N | PHE | A | 45 | 27.900 | −25.257 | −6.887 | 1.00 | 58.23 | N |
| ATOM | 222 | CA | PHE | A | 45 | 26.845 | −25.183 | −7.888 | 1.00 | 58.33 | C |
| ATOM | 223 | CB | PHE | A | 45 | 27.270 | −24.285 | −9.051 | 1.00 | 58.38 | C |
| ATOM | 224 | CG | PHE | A | 45 | 27.111 | −22.817 | −8.782 | 1.00 | 58.62 | C |
| ATOM | 225 | CD1 | PHE | A | 45 | 25.873 | −22.207 | −8.899 | 1.00 | 58.76 | C |
| ATOM | 226 | CE1 | PHE | A | 45 | 25.725 | −20.851 | −8.657 | 1.00 | 58.85 | C |
| ATOM | 227 | CZ | PHE | A | 45 | 26.822 | −20.087 | −8.301 | 1.00 | 58.65 | C |
| ATOM | 228 | CE2 | PHE | A | 45 | 28.063 | −20.681 | −8.189 | 1.00 | 58.80 | C |
| ATOM | 229 | CD2 | PHE | A | 45 | 28.205 | −22.039 | −8.429 | 1.00 | 58.92 | C |
| ATOM | 230 | C | PHE | A | 45 | 26.447 | −26.549 | −8.432 | 1.00 | 58.40 | C |
| ATOM | 231 | O | PHE | A | 45 | 25.269 | −26.809 | −8.653 | 1.00 | 58.46 | O |
| ATOM | 232 | N | HIS | A | 46 | 27.424 | −27.415 | −8.669 | 1.00 | 58.47 | N |
| ATOM | 233 | CA | HIS | A | 46 | 27.135 | −28.696 | −9.298 | 1.00 | 58.57 | C |
| ATOM | 234 | CB | HIS | A | 46 | 27.553 | −28.677 | −10.765 | 1.00 | 58.52 | C |
| ATOM | 235 | CG | HIS | A | 46 | 27.012 | −27.510 | −11.526 | 1.00 | 58.40 | C |
| ATOM | 236 | ND1 | HIS | A | 46 | 25.756 | −27.510 | −12.093 | 1.00 | 58.69 | N |
| ATOM | 237 | CE1 | HIS | A | 46 | 25.547 | −26.351 | −12.692 | 1.00 | 58.73 | C |
| ATOM | 238 | NE2 | HIS | A | 46 | 26.622 | −25.599 | −12.532 | 1.00 | 58.60 | N |
| ATOM | 239 | CD2 | HIS | A | 46 | 27.553 | −26.301 | −11.806 | 1.00 | 58.47 | C |
| ATOM | 240 | C | HIS | A | 46 | 27.804 | −29.851 | −8.577 | 1.00 | 58.82 | C |
| ATOM | 241 | O | HIS | A | 46 | 27.131 | −30.724 | −8.041 | 1.00 | 58.94 | O |
| ATOM | 242 | N | SER | A | 47 | 29.130 | −29.859 | −8.561 | 1.00 | 59.14 | N |
| ATOM | 243 | CA | SER | A | 47 | 29.856 | −30.936 | −7.907 | 1.00 | 59.57 | C |
| ATOM | 244 | CB | SER | A | 47 | 30.256 | −32.001 | −8.924 | 1.00 | 59.49 | C |
| ATOM | 245 | OG | SER | A | 47 | 29.254 | −32.992 | −9.030 | 1.00 | 59.64 | O |
| ATOM | 246 | C | SER | A | 47 | 31.089 | −30.454 | −7.160 | 1.00 | 59.92 | C |
| ATOM | 247 | O | SER | A | 47 | 31.799 | −29.566 | −7.632 | 1.00 | 60.08 | O |
| ATOM | 248 | N | PRO | A | 48 | 31.348 | −31.043 | −5.984 | 1.00 | 60.20 | N |
| ATOM | 249 | CA | PRO | A | 48 | 32.579 | −30.774 | −5.254 | 1.00 | 60.54 | C |
| ATOM | 250 | CB | PRO | A | 48 | 32.350 | −31.470 | −3.916 | 1.00 | 60.46 | C |
| ATOM | 251 | CG | PRO | A | 48 | 31.373 | −32.544 | −4.219 | 1.00 | 60.43 | C |
| ATOM | 252 | CD | PRO | A | 48 | 30.483 | −32.004 | −5.283 | 1.00 | 60.14 | C |
| ATOM | 253 | C | PRO | A | 48 | 33.749 | −31.409 | −5.991 | 1.00 | 60.95 | C |
| ATOM | 254 | O | PRO | A | 48 | 33.537 | −32.221 | −6.893 | 1.00 | 61.07 | O |
| ATOM | 255 | N | ALA | A | 49 | 34.970 | −31.039 | −5.624 | 1.00 | 61.39 | N |
| ATOM | 256 | CA | ALA | A | 49 | 36.149 | −31.565 | −6.306 | 1.00 | 61.81 | C |
| ATOM | 257 | CB | ALA | A | 49 | 37.387 | −30.779 | −5.919 | 1.00 | 61.89 | C |
| ATOM | 258 | C | ALA | A | 49 | 36.341 | −33.046 | −6.011 | 1.00 | 62.05 | C |
| ATOM | 259 | O | ALA | A | 49 | 36.042 | −33.508 | −4.914 | 1.00 | 61.90 | O |
| ATOM | 260 | N | LEU | A | 50 | 36.846 | −33.778 | −6.999 | 1.00 | 62.63 | N |
| ATOM | 261 | CA | LEU | A | 50 | 37.017 | −35.224 | −6.900 | 1.00 | 63.26 | C |
| ATOM | 262 | CB | LEU | A | 50 | 37.169 | −35.826 | −8.301 | 1.00 | 63.16 | C |
| ATOM | 263 | CG | LEU | A | 50 | 36.104 | −35.424 | −9.330 | 1.00 | 63.07 | C |
| ATOM | 264 | CD1 | LEU | A | 50 | 34.733 | −35.875 | −8.873 | 1.00 | 63.12 | C |
| ATOM | 265 | CD2 | LEU | A | 50 | 36.410 | −35.978 | −10.717 | 1.00 | 62.96 | C |
| ATOM | 266 | C | LEU | A | 50 | 38.213 | −35.607 | −6.020 | 1.00 | 63.95 | C |
| ATOM | 267 | O | LEU | A | 50 | 39.139 | −34.817 | −5.836 | 1.00 | 64.09 | O |
| ATOM | 268 | N | GLU | A | 51 | 38.181 | −36.820 | −5.469 | 1.00 | 64.70 | N |
| ATOM | 269 | CA | GLU | A | 51 | 39.296 | −37.346 | −4.683 | 1.00 | 65.43 | C |
| ATOM | 270 | CB | GLU | A | 51 | 38.780 | −37.991 | −3.401 | 1.00 | 65.54 | C |
| ATOM | 271 | CG | GLU | A | 51 | 37.576 | −37.302 | −2.788 | 1.00 | 66.38 | C |
| ATOM | 272 | CD | GLU | A | 51 | 36.647 | −38.289 | −2.106 | 1.00 | 67.59 | C |
| ATOM | 273 | OE1 | GLU | A | 51 | 36.443 | −39.386 | −2.671 | 1.00 | 68.33 | O |
| ATOM | 274 | OE2 | GLU | A | 51 | 36.120 | −37.976 | −1.013 | 1.00 | 68.07 | O |
| ATOM | 275 | C | GLU | A | 51 | 40.026 | −38.398 | −5.510 | 1.00 | 65.74 | C |
| ATOM | 276 | O | GLU | A | 51 | 39.716 | −38.592 | −6.682 | 1.00 | 65.83 | O |
| ATOM | 277 | N | ASP | A | 52 | 40.989 | −39.086 | −4.906 | 1.00 | 66.11 | N |
| ATOM | 278 | CA | ASP | A | 52 | 41.614 | −40.222 | −5.571 | 1.00 | 66.50 | C |
| ATOM | 279 | CB | ASP | A | 52 | 42.961 | −40.543 | −4.936 | 1.00 | 66.58 | C |
| ATOM | 280 | CG | ASP | A | 52 | 44.008 | −39.495 | −5.242 | 1.00 | 67.19 | C |
| ATOM | 281 | OD1 | ASP | A | 52 | 43.632 | −38.386 | −5.689 | 1.00 | 67.73 | O |
| ATOM | 282 | OD2 | ASP | A | 52 | 45.210 | −39.782 | −5.040 | 1.00 | 67.86 | O |
| ATOM | 283 | C | ASP | A | 52 | 40.687 | −41.420 | −5.465 | 1.00 | 66.69 | C |
| ATOM | 284 | O | ASP | A | 52 | 40.773 | −42.377 | −6.245 | 1.00 | 66.73 | O |
| ATOM | 285 | N | ALA | A | 53 | 39.783 | −41.341 | −4.495 | 1.00 | 66.92 | N |
| ATOM | 286 | CA | ALA | A | 53 | 38.878 | −42.436 | −4.184 | 1.00 | 67.19 | C |
| ATOM | 287 | CB | ALA | A | 53 | 38.099 | −42.128 | −2.914 | 1.00 | 67.25 | C |
| ATOM | 288 | C | ALA | A | 53 | 37.930 | −42.772 | −5.331 | 1.00 | 67.26 | C |
| ATOM | 289 | O | ALA | A | 53 | 37.550 | −43.926 | −5.503 | 1.00 | 67.24 | O |
| ATOM | 290 | N | ASP | A | 54 | 37.543 | −41.771 | −6.113 | 1.00 | 67.45 | N |
| ATOM | 291 | CA | ASP | A | 54 | 36.663 | −42.028 | −7.251 | 1.00 | 67.74 | C |
| ATOM | 292 | CB | ASP | A | 54 | 35.641 | −40.899 | −7.467 | 1.00 | 67.90 | C |
| ATOM | 293 | CG | ASP | A | 54 | 36.161 | −39.544 | −7.036 | 1.00 | 68.67 | C |
| ATOM | 294 | OD1 | ASP | A | 54 | 37.398 | −39.378 | −6.970 | 1.00 | 69.79 | O |
| ATOM | 295 | OD2 | ASP | A | 54 | 35.333 | −38.646 | −6.758 | 1.00 | 69.03 | O |
| ATOM | 296 | C | ASP | A | 54 | 37.458 | −42.329 | −8.520 | 1.00 | 67.62 | C |
| ATOM | 297 | O | ASP | A | 54 | 36.900 | −42.378 | −9.618 | 1.00 | 67.85 | O |
| ATOM | 298 | N | PHE | A | 55 | 38.762 | −42.532 | −8.353 | 1.00 | 67.33 | N |
| ATOM | 299 | CA | PHE | A | 55 | 39.616 | −42.994 | −9.442 | 1.00 | 67.04 | C |
| ATOM | 300 | CB | PHE | A | 55 | 40.806 | −42.056 | −9.639 | 1.00 | 67.01 | C |
| ATOM | 301 | CG | PHE | A | 55 | 40.448 | −40.743 | −10.259 | 1.00 | 66.79 | C |
| ATOM | 302 | CD1 | PHE | A | 55 | 40.078 | −39.670 | −9.471 | 1.00 | 66.59 | C |
| ATOM | 303 | CE1 | PHE | A | 55 | 39.754 | −38.459 | −10.037 | 1.00 | 66.30 | C |
| ATOM | 304 | CZ | PHE | A | 55 | 39.800 | −38.306 | −11.403 | 1.00 | 66.49 | C |
| ATOM | 305 | CE2 | PHE | A | 55 | 40.171 | −39.366 | −12.200 | 1.00 | 66.62 | C |
| ATOM | 306 | CD2 | PHE | A | 55 | 40.495 | −40.576 | −11.628 | 1.00 | 66.65 | C |
| ATOM | 307 | C | PHE | A | 55 | 40.136 | −44.396 | −9.151 | 1.00 | 66.92 | C |
| ATOM | 308 | O | PHE | A | 55 | 40.288 | −45.209 | −10.061 | 1.00 | 66.88 | O |
| ATOM | 309 | N | ASP | A | 56 | 40.415 | −44.669 | −7.879 | 1.00 | 66.74 | N |
| ATOM | 310 | CA | ASP | A | 56 | 41.024 | −45.940 | −7.491 | 1.00 | 66.60 | C |
| ATOM | 311 | CB | ASP | A | 56 | 42.316 | −45.690 | −6.707 | 1.00 | 66.71 | C |
| ATOM | 312 | CG | ASP | A | 56 | 43.344 | −44.905 | −7.504 | 1.00 | 67.09 | C |
| ATOM | 313 | OD1 | ASP | A | 56 | 43.364 | −45.043 | −8.747 | 1.00 | 67.21 | O |
| ATOM | 314 | OD2 | ASP | A | 56 | 44.135 | −44.153 | −6.886 | 1.00 | 67.60 | O |
| ATOM | 315 | C | ASP | A | 56 | 40.091 | −46.835 | −6.674 | 1.00 | 66.36 | C |
| ATOM | 316 | O | ASP | A | 56 | 40.544 | −47.576 | −5.801 | 1.00 | 66.42 | O |
| ATOM | 317 | N | GLY | A | 57 | 38.796 | −46.777 | −6.961 | 1.00 | 66.00 | N |
| ATOM | 318 | CA | GLY | A | 57 | 37.819 | −47.528 | −6.182 | 1.00 | 65.67 | C |
| ATOM | 319 | C | GLY | A | 57 | 37.046 | −48.534 | −7.006 | 1.00 | 65.47 | C |
| ATOM | 320 | O | GLY | A | 57 | 36.955 | −48.408 | −8.227 | 1.00 | 65.50 | O |
| ATOM | 321 | N | LYS | A | 58 | 36.485 | −49.535 | −6.331 | 1.00 | 65.22 | N |
| ATOM | 322 | CA | LYS | A | 58 | 35.741 | −50.602 | −7.000 | 1.00 | 64.95 | C |
| ATOM | 323 | CB | LYS | A | 58 | 35.422 | −51.734 | −6.019 | 1.00 | 64.95 | C |
| ATOM | 324 | CG | LYS | A | 58 | 36.409 | −52.886 | −6.068 | 1.00 | 65.08 | C |
| ATOM | 325 | CD | LYS | A | 58 | 36.419 | −53.661 | −4.763 | 1.00 | 65.47 | C |
| ATOM | 326 | CE | LYS | A | 58 | 35.019 | −54.076 | −4.348 | 1.00 | 65.82 | C |
| ATOM | 327 | NZ | LYS | A | 58 | 35.015 | −54.712 | −2.998 | 1.00 | 66.10 | N |
| ATOM | 328 | C | LYS | A | 58 | 34.458 | −50.095 | −7.648 | 1.00 | 64.68 | C |
| ATOM | 329 | O | LYS | A | 58 | 33.691 | −49.365 | −7.019 | 1.00 | 64.72 | O |
| ATOM | 330 | N | PRO | A | 59 | 34.226 | −50.484 | −8.913 | 1.00 | 64.36 | N |
| ATOM | 331 | CA | PRO | A | 59 | 33.020 | −50.095 | −9.642 | 1.00 | 64.14 | C |
| ATOM | 332 | CB | PRO | A | 59 | 33.087 | −50.937 | −10.927 | 1.00 | 64.07 | C |
| ATOM | 333 | CG | PRO | A | 59 | 34.179 | −51.939 | −10.707 | 1.00 | 64.14 | C |
| ATOM | 334 | CD | PRO | A | 59 | 35.118 | −51.317 | −9.734 | 1.00 | 64.28 | C |
| ATOM | 335 | C | PRO | A | 59 | 31.740 | −50.401 | −8.865 | 1.00 | 63.96 | C |
| ATOM | 336 | O | PRO | A | 59 | 31.643 | −51.438 | −8.205 | 1.00 | 63.99 | O |
| ATOM | 337 | N | MET | A | 60 | 30.777 | −49.488 | −8.942 | 1.00 | 63.71 | N |
| ATOM | 338 | CA | MET | A | 60 | 29.481 | −49.666 | −8.301 | 1.00 | 63.49 | C |
| ATOM | 339 | CB | MET | A | 60 | 29.069 | −48.382 | −7.572 | 1.00 | 63.41 | C |
| ATOM | 340 | CG | MET | A | 60 | 29.977 | −47.957 | −6.428 | 1.00 | 63.33 | C |
| ATOM | 341 | SD | MET | A | 60 | 30.125 | −46.152 | −6.292 | 1.00 | 63.57 | S |
| ATOM | 342 | CE | MET | A | 60 | 30.571 | −45.961 | −4.564 | 1.00 | 63.70 | C |
| ATOM | 343 | C | MET | A | 60 | 28.435 | −49.986 | −9.363 | 1.00 | 63.31 | C |
| ATOM | 344 | O | MET | A | 60 | 28.549 | −49.526 | −10.496 | 1.00 | 63.29 | O |
| ATOM | 345 | N | VAL | A | 61 | 27.428 | −50.779 | −9.012 | 1.00 | 63.07 | N |
| ATOM | 346 | CA | VAL | A | 61 | 26.204 | −50.802 | −9.810 | 1.00 | 62.97 | C |
| ATOM | 347 | CB | VAL | A | 61 | 25.898 | −52.180 | −10.449 | 1.00 | 62.87 | C |
| ATOM | 348 | CG1 | VAL | A | 61 | 27.161 | −52.805 | −10.997 | 1.00 | 62.90 | C |
| ATOM | 349 | CG2 | VAL | A | 61 | 25.241 | −53.104 | −9.458 | 1.00 | 62.98 | C |
| ATOM | 350 | C | VAL | A | 61 | 25.049 | −50.335 | −8.929 | 1.00 | 63.00 | C |
| ATOM | 351 | O | VAL | A | 61 | 25.021 | −50.612 | −7.722 | 1.00 | 63.16 | O |
| ATOM | 352 | N | LEU | A | 62 | 24.115 | −49.599 | −9.523 | 1.00 | 62.78 | N |
| ATOM | 353 | CA | LEU | A | 62 | 22.993 | −49.062 | −8.769 | 1.00 | 62.54 | C |
| ATOM | 354 | CB | LEU | A | 62 | 22.929 | −47.541 | −8.896 | 1.00 | 62.51 | C |
| ATOM | 355 | CG | LEU | A | 62 | 21.760 | −46.869 | −8.168 | 1.00 | 62.61 | C |
| ATOM | 356 | CD1 | LEU | A | 62 | 22.157 | −45.506 | −7.620 | 1.00 | 62.58 | C |
| ATOM | 357 | CD2 | LEU | A | 62 | 20.545 | −46.751 | −9.077 | 1.00 | 62.83 | C |
| ATOM | 358 | C | LEU | A | 62 | 21.684 | −49.675 | −9.218 | 1.00 | 62.41 | C |
| ATOM | 359 | O | LEU | A | 62 | 21.380 | −49.715 | −10.410 | 1.00 | 62.37 | O |
| ATOM | 360 | N | VAL | A | 63 | 20.907 | −50.148 | −8.252 | 1.00 | 62.32 | N |
| ATOM | 361 | CA | VAL | A | 63 | 19.608 | −50.731 | −8.544 | 1.00 | 62.19 | C |
| ATOM | 362 | CB | VAL | A | 63 | 19.468 | −52.128 | −7.927 | 1.00 | 62.15 | C |
| ATOM | 363 | CG1 | VAL | A | 63 | 18.208 | −52.787 | −8.436 | 1.00 | 62.50 | C |
| ATOM | 364 | CG2 | VAL | A | 63 | 20.684 | −52.982 | −8.265 | 1.00 | 61.99 | C |
| ATOM | 365 | C | VAL | A | 63 | 18.481 | −49.830 | −8.046 | 1.00 | 62.07 | C |
| ATOM | 366 | O | VAL | A | 63 | 18.360 | −49.577 | −6.847 | 1.00 | 61.99 | O |
| ATOM | 367 | N | ALA | A | 64 | 17.666 | −49.344 | −8.978 | 1.00 | 62.00 | N |
| ATOM | 368 | CA | ALA | A | 64 | 16.562 | −48.446 | −8.651 | 1.00 | 61.93 | C |
| ATOM | 369 | CB | ALA | A | 64 | 16.855 | −47.043 | −9.141 | 1.00 | 61.86 | C |
| ATOM | 370 | C | ALA | A | 64 | 15.266 | −48.949 | −9.257 | 1.00 | 61.87 | C |
| ATOM | 371 | O | ALA | A | 64 | 15.275 | −49.792 | −10.146 | 1.00 | 62.00 | O |
| ATOM | 372 | N | GLY | A | 65 | 14.153 | −48.418 | −8.777 | 1.00 | 61.82 | N |
| ATOM | 373 | CA | GLY | A | 65 | 12.838 | −48.820 | −9.250 | 1.00 | 62.04 | C |
| ATOM | 374 | C | GLY | A | 65 | 11.837 | −48.413 | −8.194 | 1.00 | 62.30 | C |
| ATOM | 375 | O | GLY | A | 65 | 12.228 | −47.955 | −7.118 | 1.00 | 62.42 | O |
| ATOM | 376 | N | GLN | A | 66 | 10.549 | −48.562 | −8.478 | 1.00 | 62.45 | N |
| ATOM | 377 | CA | GLN | A | 66 | 9.560 | −48.233 | −7.464 | 1.00 | 62.73 | C |
| ATOM | 378 | CB | GLN | A | 66 | 8.301 | −47.616 | −8.067 | 1.00 | 62.81 | C |
| ATOM | 379 | CG | GLN | A | 66 | 7.858 | −48.190 | −9.387 | 1.00 | 63.71 | C |
| ATOM | 380 | CD | GLN | A | 66 | 6.754 | −47.355 | −10.012 | 1.00 | 64.68 | C |
| ATOM | 381 | OE1 | GLN | A | 66 | 5.905 | −46.794 | −9.305 | 1.00 | 65.17 | O |
| ATOM | 382 | NE2 | GLN | A | 66 | 6.760 | −47.264 | −11.340 | 1.00 | 65.07 | N |
| ATOM | 383 | C | GLN | A | 66 | 9.240 | −49.424 | −6.577 | 1.00 | 62.78 | C |
| ATOM | 384 | O | GLN | A | 66 | 9.877 | −50.470 | −6.685 | 1.00 | 62.88 | O |
| ATOM | 385 | N | TYR | A | 67 | 8.263 | −49.258 | −5.694 | 1.00 | 62.88 | N |
| ATOM | 386 | CA | TYR | A | 67 | 7.972 | −50.258 | −4.673 | 1.00 | 62.98 | C |
| ATOM | 387 | CB | TYR | A | 67 | 6.770 | −49.831 | −3.825 | 1.00 | 63.32 | C |
| ATOM | 388 | CG | TYR | A | 67 | 7.016 | −48.619 | −2.953 | 1.00 | 63.88 | C |
| ATOM | 389 | CD1 | TYR | A | 67 | 6.105 | −47.566 | −2.918 | 1.00 | 64.41 | C |
| ATOM | 390 | CE1 | TYR | A | 67 | 6.324 | −46.451 | −2.112 | 1.00 | 64.56 | C |
| ATOM | 391 | CZ | TYR | A | 67 | 7.470 | −46.381 | −1.336 | 1.00 | 64.29 | C |
| ATOM | 392 | OH | TYR | A | 67 | 7.695 | −45.278 | −0.537 | 1.00 | 64.28 | O |
| ATOM | 393 | CE2 | TYR | A | 67 | 8.389 | −47.414 | −1.356 | 1.00 | 64.30 | C |
| ATOM | 394 | CD2 | TYR | A | 67 | 8.160 | −48.526 | −2.162 | 1.00 | 64.33 | C |
| ATOM | 395 | C | TYR | A | 67 | 7.728 | −51.655 | −5.232 | 1.00 | 62.84 | C |
| ATOM | 396 | O | TYR | A | 67 | 7.053 | −51.824 | −6.246 | 1.00 | 62.73 | O |
| ATOM | 397 | N | SER | A | 68 | 8.293 | −52.648 | −4.550 | 1.00 | 62.73 | N |
| ATOM | 398 | CA | SER | A | 68 | 8.040 | −54.060 | −4.830 | 1.00 | 62.58 | C |
| ATOM | 399 | CB | SER | A | 68 | 6.600 | −54.438 | −4.459 | 1.00 | 62.63 | C |
| ATOM | 400 | OG | SER | A | 68 | 6.339 | −54.184 | −3.086 | 1.00 | 62.93 | O |
| ATOM | 401 | C | SER | A | 68 | 8.328 | −54.439 | −6.272 | 1.00 | 62.40 | C |
| ATOM | 402 | O | SER | A | 68 | 7.649 | −55.288 | −6.848 | 1.00 | 62.68 | O |
| ATOM | 403 | N | THR | A | 69 | 9.338 | −53.814 | −6.858 | 1.00 | 61.97 | N |
| ATOM | 404 | CA | THR | A | 69 | 9.697 | −54.129 | −8.225 | 1.00 | 61.63 | C |
| ATOM | 405 | CB | THR | A | 69 | 10.267 | −52.907 | −8.953 | 1.00 | 61.73 | C |
| ATOM | 406 | OG1 | THR | A | 69 | 9.623 | −51.719 | −8.480 | 1.00 | 61.90 | O |
| ATOM | 407 | CG2 | THR | A | 69 | 10.026 | −53.030 | −10.435 | 1.00 | 62.25 | C |
| ATOM | 408 | C | THR | A | 69 | 10.712 | −55.266 | −8.232 | 1.00 | 61.26 | C |
| ATOM | 409 | O | THR | A | 69 | 11.031 | −55.832 | −9.276 | 1.00 | 61.25 | O |
| ATOM | 410 | N | GLY | A | 70 | 11.222 | −55.596 | −7.053 | 1.00 | 60.94 | N |
| ATOM | 411 | CA | GLY | A | 70 | 12.143 | −56.715 | −6.910 | 1.00 | 60.52 | C |
| ATOM | 412 | C | GLY | A | 70 | 13.612 | −56.350 | −6.934 | 1.00 | 60.22 | C |
| ATOM | 413 | O | GLY | A | 70 | 14.435 | −57.156 | −7.334 | 1.00 | 60.21 | O |
| ATOM | 414 | N | LYS | A | 71 | 13.950 | −55.138 | −6.507 | 1.00 | 60.02 | N |
| ATOM | 415 | CA | LYS | A | 71 | 15.344 | −54.712 | −6.478 | 1.00 | 59.94 | C |
| ATOM | 416 | CB | LYS | A | 71 | 15.440 | −53.272 | −5.986 | 1.00 | 59.83 | C |
| ATOM | 417 | CG | LYS | A | 71 | 14.957 | −52.237 | −6.980 | 1.00 | 59.55 | C |
| ATOM | 418 | CD | LYS | A | 71 | 14.638 | −50.922 | −6.287 | 1.00 | 58.81 | C |
| ATOM | 419 | CE | LYS | A | 71 | 13.242 | −50.955 | −5.692 | 1.00 | 58.14 | C |
| ATOM | 420 | NZ | LYS | A | 71 | 13.111 | −50.011 | −4.562 | 1.00 | 57.62 | N |
| ATOM | 421 | C | LYS | A | 71 | 16.170 | −55.620 | −5.570 | 1.00 | 60.06 | C |
| ATOM | 422 | O | LYS | A | 71 | 17.156 | −56.244 | −5.998 | 1.00 | 60.25 | O |
| ATOM | 423 | N | THR | A | 72 | 15.754 | −55.686 | −4.309 | 1.00 | 60.04 | N |
| ATOM | 424 | CA | THR | A | 72 | 16.436 | −56.494 | −3.314 | 1.00 | 60.09 | C |
| ATOM | 425 | CB | THR | A | 72 | 15.698 | −56.451 | −1.955 | 1.00 | 60.10 | C |
| ATOM | 426 | OG1 | THR | A | 72 | 15.224 | −55.121 | −1.703 | 1.00 | 60.02 | O |
| ATOM | 427 | CG2 | THR | A | 72 | 16.626 | −56.878 | −0.824 | 1.00 | 60.05 | C |
| ATOM | 428 | C | THR | A | 72 | 16.548 | −57.931 | −3.814 | 1.00 | 60.14 | C |
| ATOM | 429 | O | THR | A | 72 | 17.605 | −58.562 | −3.702 | 1.00 | 60.40 | O |
| ATOM | 430 | N | SER | A | 73 | 15.457 | −58.438 | −4.382 | 1.00 | 60.12 | N |
| ATOM | 431 | CA | SER | A | 73 | 15.446 | −59.783 | −4.939 | 1.00 | 60.22 | C |
| ATOM | 432 | CB | SER | A | 73 | 14.064 | −60.131 | −5.502 | 1.00 | 60.26 | C |
| ATOM | 433 | OG | SER | A | 73 | 13.120 | −60.336 | −4.462 | 1.00 | 59.98 | O |
| ATOM | 434 | C | SER | A | 73 | 16.501 | −59.901 | −6.027 | 1.00 | 60.19 | C |
| ATOM | 435 | O | SER | A | 73 | 17.298 | −60.825 | −6.042 | 1.00 | 60.56 | O |
| ATOM | 436 | N | PHE | A | 74 | 16.496 | −58.942 | −6.936 | 1.00 | 60.20 | N |
| ATOM | 437 | CA | PHE | A | 74 | 17.456 | −58.885 | −8.016 | 1.00 | 60.14 | C |
| ATOM | 438 | CB | PHE | A | 74 | 17.294 | −57.558 | −8.748 | 1.00 | 60.03 | C |
| ATOM | 439 | CG | PHE | A | 74 | 18.223 | −57.383 | −9.900 | 1.00 | 60.15 | C |
| ATOM | 440 | CD1 | PHE | A | 74 | 18.013 | −58.066 | −11.083 | 1.00 | 60.37 | C |
| ATOM | 441 | CE1 | PHE | A | 74 | 18.866 | −57.899 | −12.147 | 1.00 | 60.41 | C |
| ATOM | 442 | CZ | PHE | A | 74 | 19.937 | −57.033 | −12.040 | 1.00 | 60.50 | C |
| ATOM | 443 | CE2 | PHE | A | 74 | 20.148 | −56.342 | −10.870 | 1.00 | 60.12 | C |
| ATOM | 444 | CD2 | PHE | A | 74 | 19.297 | −56.517 | −9.810 | 1.00 | 60.02 | C |
| ATOM | 445 | C | PHE | A | 74 | 18.876 | −59.038 | −7.486 | 1.00 | 60.19 | C |
| ATOM | 446 | O | PHE | A | 74 | 19.623 | −59.913 | −7.925 | 1.00 | 60.38 | O |
| ATOM | 447 | N | ILE | A | 75 | 19.247 | −58.192 | −6.532 | 1.00 | 60.27 | N |
| ATOM | 448 | CA | ILE | A | 75 | 20.599 | −58.260 | −5.988 | 1.00 | 60.40 | C |
| ATOM | 449 | CB | ILE | A | 75 | 20.862 | −57.131 | −4.989 | 1.00 | 60.26 | C |
| ATOM | 450 | CG1 | ILE | A | 75 | 20.540 | −55.781 | −5.625 | 1.00 | 60.08 | C |
| ATOM | 451 | CD1 | ILE | A | 75 | 20.666 | −54.622 | −4.673 | 1.00 | 59.79 | C |
| ATOM | 452 | CG2 | ILE | A | 75 | 22.305 | −57.159 | −4.530 | 1.00 | 60.22 | C |
| ATOM | 453 | C | ILE | A | 75 | 20.850 | −59.613 | −5.324 | 1.00 | 60.71 | C |
| ATOM | 454 | O | ILE | A | 75 | 21.862 | −60.278 | −5.584 | 1.00 | 61.02 | O |
| ATOM | 455 | N | GLN | A | 76 | 19.915 | −60.007 | −4.464 | 1.00 | 60.91 | N |
| ATOM | 456 | CA | GLN | A | 76 | 19.933 | −61.311 | −3.814 | 1.00 | 61.22 | C |
| ATOM | 457 | CB | GLN | A | 76 | 18.538 | −61.595 | −3.263 | 1.00 | 61.23 | C |
| ATOM | 458 | CG | GLN | A | 76 | 18.435 | −62.776 | −2.330 | 1.00 | 62.03 | C |
| ATOM | 459 | CD | GLN | A | 76 | 18.200 | −62.353 | −0.894 | 1.00 | 63.06 | C |
| ATOM | 460 | OE1 | GLN | A | 76 | 17.409 | −62.972 | −0.176 | 1.00 | 63.47 | O |
| ATOM | 461 | NE2 | GLN | A | 76 | 18.873 | −61.282 | −0.471 | 1.00 | 63.36 | N |
| ATOM | 462 | C | GLN | A | 76 | 20.292 | −62.381 | −4.840 | 1.00 | 61.30 | C |
| ATOM | 463 | O | GLN | A | 76 | 21.096 | −63.280 | −4.589 | 1.00 | 61.68 | O |
| ATOM | 464 | N | TYR | A | 77 | 19.678 | −62.242 | −6.009 | 1.00 | 61.31 | N |
| ATOM | 465 | CA | TYR | A | 77 | 19.786 | −63.181 | −7.115 | 1.00 | 61.46 | C |
| ATOM | 466 | CB | TYR | A | 77 | 18.682 | −62.874 | −8.125 | 1.00 | 61.45 | C |
| ATOM | 467 | CG | TYR | A | 77 | 18.636 | −63.776 | −9.326 | 1.00 | 61.46 | C |
| ATOM | 468 | CD1 | TYR | A | 77 | 18.978 | −63.300 | −10.583 | 1.00 | 61.59 | C |
| ATOM | 469 | CE1 | TYR | A | 77 | 18.919 | −64.117 | −11.698 | 1.00 | 61.89 | C |
| ATOM | 470 | CZ | TYR | A | 77 | 18.512 | −65.428 | −11.559 | 1.00 | 61.81 | C |
| ATOM | 471 | OH | TYR | A | 77 | 18.455 | −66.244 | −12.661 | 1.00 | 61.74 | O |
| ATOM | 472 | CE2 | TYR | A | 77 | 18.163 | −65.923 | −10.318 | 1.00 | 61.90 | C |
| ATOM | 473 | CD2 | TYR | A | 77 | 18.227 | −65.097 | −9.212 | 1.00 | 61.71 | C |
| ATOM | 474 | C | TYR | A | 77 | 21.145 | −63.093 | −7.791 | 1.00 | 61.48 | C |
| ATOM | 475 | O | TYR | A | 77 | 21.713 | −64.112 | −8.184 | 1.00 | 61.51 | O |
| ATOM | 476 | N | LEU | A | 78 | 21.659 | −61.874 | −7.936 | 1.00 | 61.31 | N |
| ATOM | 477 | CA | LEU | A | 78 | 22.991 | −61.687 | −8.493 | 1.00 | 61.65 | C |
| ATOM | 478 | CB | LEU | A | 78 | 23.306 | −60.206 | −8.664 | 1.00 | 61.70 | C |
| ATOM | 479 | CG | LEU | A | 78 | 22.978 | −59.629 | −10.039 | 1.00 | 62.28 | C |
| ATOM | 480 | CD1 | LEU | A | 78 | 23.121 | −58.112 | −10.024 | 1.00 | 62.88 | C |
| ATOM | 481 | CD2 | LEU | A | 78 | 23.908 | −60.242 | −11.070 | 1.00 | 62.72 | C |
| ATOM | 482 | C | LEU | A | 78 | 24.031 | −62.325 | −7.596 | 1.00 | 61.78 | C |
| ATOM | 483 | O | LEU | A | 78 | 24.988 | −62.939 | −8.069 | 1.00 | 61.79 | O |
| ATOM | 484 | N | LEU | A | 79 | 23.837 | −62.181 | −6.292 | 1.00 | 61.84 | N |
| ATOM | 485 | CA | LEU | A | 79 | 24.798 | −62.702 | −5.341 | 1.00 | 62.22 | C |
| ATOM | 486 | CB | LEU | A | 79 | 24.754 | −61.888 | −4.051 | 1.00 | 62.29 | C |
| ATOM | 487 | CG | LEU | A | 79 | 25.012 | −60.386 | −4.176 | 1.00 | 62.42 | C |
| ATOM | 488 | CD1 | LEU | A | 79 | 25.014 | −59.736 | −2.800 | 1.00 | 62.58 | C |
| ATOM | 489 | CD2 | LEU | A | 79 | 26.324 | −60.120 | −4.896 | 1.00 | 62.69 | C |
| ATOM | 490 | C | LEU | A | 79 | 24.547 | −64.168 | −5.032 | 1.00 | 62.47 | C |
| ATOM | 491 | O | LEU | A | 79 | 25.397 | −64.833 | −4.439 | 1.00 | 62.39 | O |
| ATOM | 492 | N | GLU | A | 80 | 23.383 | −64.668 | −5.440 | 1.00 | 62.76 | N |
| ATOM | 493 | CA | GLU | A | 80 | 22.942 | −66.001 | −5.034 | 1.00 | 63.58 | C |
| ATOM | 494 | CB | GLU | A | 80 | 23.637 | −67.091 | −5.852 | 1.00 | 63.72 | C |
| ATOM | 495 | CG | GLU | A | 80 | 23.051 | −67.293 | −7.250 | 1.00 | 65.58 | C |
| ATOM | 496 | CD | GLU | A | 80 | 22.042 | −68.443 | −7.324 | 1.00 | 68.13 | C |
| ATOM | 497 | OE1 | GLU | A | 80 | 22.412 | −69.522 | −7.867 | 1.00 | 69.12 | O |
| ATOM | 498 | OE2 | GLU | A | 80 | 20.888 | −68.278 | −6.838 | 1.00 | 68.88 | O |
| ATOM | 499 | C | GLU | A | 80 | 23.214 | −66.180 | −3.544 | 1.00 | 63.57 | C |
| ATOM | 500 | O | GLU | A | 80 | 23.706 | −67.219 | −3.106 | 1.00 | 63.47 | O |
| ATOM | 501 | N | GLN | A | 81 | 22.899 | −65.133 | −2.784 | 1.00 | 63.78 | N |
| ATOM | 502 | CA | GLN | A | 81 | 23.086 | −65.106 | −1.342 | 1.00 | 63.93 | C |
| ATOM | 503 | CB | GLN | A | 81 | 24.562 | −64.900 | −0.993 | 1.00 | 63.84 | C |
| ATOM | 504 | CG | GLN | A | 81 | 24.847 | −64.947 | 0.504 | 1.00 | 63.39 | C |
| ATOM | 505 | CD | GLN | A | 81 | 26.324 | −64.864 | 0.824 | 1.00 | 62.75 | C |
| ATOM | 506 | OE1 | GLN | A | 81 | 26.739 | −65.199 | 1.929 | 1.00 | 63.13 | O |
| ATOM | 507 | NE2 | GLN | A | 81 | 27.127 | −64.417 | −0.137 | 1.00 | 62.73 | N |
| ATOM | 508 | C | GLN | A | 81 | 22.239 | −63.987 | −0.731 | 1.00 | 64.17 | C |
| ATOM | 509 | O | GLN | A | 81 | 22.026 | −62.952 | −1.361 | 1.00 | 64.15 | O |
| ATOM | 510 | N | GLU | A | 82 | 21.757 | −64.204 | 0.492 | 1.00 | 64.47 | N |
| ATOM | 511 | CA | GLU | A | 82 | 20.916 | −63.227 | 1.182 | 1.00 | 64.72 | C |
| ATOM | 512 | CB | GLU | A | 82 | 20.083 | −63.910 | 2.277 | 1.00 | 64.74 | C |
| ATOM | 513 | CG | GLU | A | 82 | 18.846 | −63.123 | 2.712 | 1.00 | 65.36 | C |
| ATOM | 514 | CD | GLU | A | 82 | 17.597 | −63.996 | 2.870 | 1.00 | 66.14 | C |
| ATOM | 515 | OE1 | GLU | A | 82 | 17.685 | −65.080 | 3.490 | 1.00 | 66.18 | O |
| ATOM | 516 | OE2 | GLU | A | 82 | 16.520 | −63.591 | 2.372 | 1.00 | 66.28 | O |
| ATOM | 517 | C | GLU | A | 82 | 21.732 | −62.057 | 1.748 | 1.00 | 64.81 | C |
| ATOM | 518 | O | GLU | A | 82 | 22.863 | −62.233 | 2.202 | 1.00 | 64.77 | O |
| ATOM | 519 | N | VAL | A | 83 | 21.152 | −60.862 | 1.705 | 1.00 | 64.99 | N |
| ATOM | 520 | CA | VAL | A | 83 | 21.825 | −59.654 | 2.169 | 1.00 | 65.07 | C |
| ATOM | 521 | CB | VAL | A | 83 | 21.294 | −58.413 | 1.439 | 1.00 | 65.06 | C |
| ATOM | 522 | CG1 | VAL | A | 83 | 22.242 | −57.244 | 1.628 | 1.00 | 65.06 | C |
| ATOM | 523 | CG2 | VAL | A | 83 | 21.098 | −58.712 | −0.036 | 1.00 | 64.87 | C |
| ATOM | 524 | C | VAL | A | 83 | 21.638 | −59.465 | 3.673 | 1.00 | 65.23 | C |
| ATOM | 525 | O | VAL | A | 83 | 20.509 | −59.404 | 4.157 | 1.00 | 65.10 | O |
| ATOM | 526 | N | PRO | A | 84 | 22.752 | −59.365 | 4.414 | 1.00 | 65.51 | N |
| ATOM | 527 | CA | PRO | A | 84 | 22.753 | −59.247 | 5.869 | 1.00 | 65.77 | C |
| ATOM | 528 | CB | PRO | A | 84 | 24.156 | −58.723 | 6.168 | 1.00 | 65.66 | C |
| ATOM | 529 | CG | PRO | A | 84 | 24.990 | −59.315 | 5.100 | 1.00 | 65.56 | C |
| ATOM | 530 | CD | PRO | A | 84 | 24.120 | −59.365 | 3.867 | 1.00 | 65.53 | C |
| ATOM | 531 | C | PRO | A | 84 | 21.706 | −58.275 | 6.401 | 1.00 | 66.15 | C |
| ATOM | 532 | O | PRO | A | 84 | 20.987 | −58.603 | 7.342 | 1.00 | 66.22 | O |
| ATOM | 533 | N | GLY | A | 85 | 21.616 | −57.094 | 5.802 | 1.00 | 66.61 | N |
| ATOM | 534 | CA | GLY | A | 85 | 20.694 | −56.072 | 6.282 | 1.00 | 67.21 | C |
| ATOM | 535 | C | GLY | A | 85 | 19.347 | −56.044 | 5.581 | 1.00 | 67.67 | C |
| ATOM | 536 | O | GLY | A | 85 | 18.320 | −55.785 | 6.211 | 1.00 | 67.61 | O |
| ATOM | 537 | N | SER | A | 86 | 19.348 | −56.313 | 4.278 | 1.00 | 68.17 | N |
| ATOM | 538 | CA | SER | A | 86 | 18.144 | −56.150 | 3.456 | 1.00 | 68.71 | C |
| ATOM | 539 | CB | SER | A | 86 | 18.514 | −55.711 | 2.031 | 1.00 | 68.74 | C |
| ATOM | 540 | OG | SER | A | 86 | 18.520 | −54.297 | 1.904 | 1.00 | 68.90 | O |
| ATOM | 541 | C | SER | A | 86 | 17.225 | −57.374 | 3.398 | 1.00 | 69.01 | C |
| ATOM | 542 | O | SER | A | 86 | 17.554 | −58.386 | 2.769 | 1.00 | 69.11 | O |
| ATOM | 543 | N | ARG | A | 87 | 16.065 | −57.263 | 4.044 | 1.00 | 69.32 | N |
| ATOM | 544 | CA | ARG | A | 87 | 15.015 | −58.266 | 3.918 | 1.00 | 69.63 | C |
| ATOM | 545 | CB | ARG | A | 87 | 14.122 | −58.274 | 5.161 | 1.00 | 69.53 | C |
| ATOM | 546 | CG | ARG | A | 87 | 15.231 | −59.653 | 5.857 | 0.00 | 50.00 | C |
| ATOM | 547 | CD | ARG | A | 87 | 14.525 | −60.115 | 7.110 | 0.00 | 50.00 | C |
| ATOM | 548 | NE | ARG | A | 87 | 13.769 | −61.343 | 6.893 | 0.00 | 50.00 | N |
| ATOM | 549 | CZ | ARG | A | 87 | 14.263 | −62.572 | 6.969 | 0.00 | 50.00 | C |
| ATOM | 550 | NH1 | ARG | A | 87 | 13.467 | −63.609 | 6.740 | 0.00 | 50.00 | N |
| ATOM | 551 | NH2 | ARG | A | 87 | 15.528 | −62.779 | 7.297 | 0.00 | 50.00 | N |
| ATOM | 552 | C | ARG | A | 87 | 14.181 | −57.999 | 2.661 | 1.00 | 69.92 | C |
| ATOM | 553 | O | ARG | A | 87 | 14.475 | −57.085 | 1.888 | 1.00 | 69.93 | O |
| ATOM | 554 | N | VAL | A | 88 | 13.143 | −58.803 | 2.461 | 1.00 | 70.32 | N |
| ATOM | 555 | CA | VAL | A | 88 | 12.278 | −58.678 | 1.289 | 1.00 | 70.65 | C |
| ATOM | 556 | CB | VAL | A | 88 | 12.785 | −59.565 | 0.124 | 1.00 | 70.63 | C |
| ATOM | 557 | CG1 | VAL | A | 88 | 11.662 | −59.875 | −0.855 | 1.00 | 70.52 | C |
| ATOM | 558 | CG2 | VAL | A | 88 | 13.398 | −60.855 | 0.661 | 1.00 | 70.76 | C |
| ATOM | 559 | C | VAL | A | 88 | 10.836 | −59.035 | 1.655 | 1.00 | 70.97 | C |
| ATOM | 560 | O | VAL | A | 88 | 10.594 | −59.997 | 2.389 | 1.00 | 71.03 | O |
| ATOM | 561 | N | GLY | A | 89 | 9.881 | −58.255 | 1.157 | 1.00 | 71.28 | N |
| ATOM | 562 | CA | GLY | A | 89 | 8.480 | −58.471 | 1.506 | 1.00 | 71.78 | C |
| ATOM | 563 | C | GLY | A | 89 | 7.485 | −57.882 | 0.527 | 1.00 | 72.17 | C |
| ATOM | 564 | O | GLY | A | 89 | 7.873 | −57.243 | −0.451 | 1.00 | 72.08 | O |
| ATOM | 565 | N | PRO | A | 90 | 6.184 | −58.087 | 0.798 | 1.00 | 72.59 | N |
| ATOM | 566 | CA | PRO | A | 90 | 5.083 | −57.648 | −0.065 | 1.00 | 72.92 | C |
| ATOM | 567 | CB | PRO | A | 90 | 3.845 | −58.251 | 0.610 | 1.00 | 72.90 | C |
| ATOM | 568 | CG | PRO | A | 90 | 4.241 | −58.441 | 2.041 | 1.00 | 72.85 | C |
| ATOM | 569 | CD | PRO | A | 90 | 5.703 | −58.771 | 2.013 | 1.00 | 72.65 | C |
| ATOM | 570 | C | PRO | A | 90 | 4.977 | −56.126 | −0.118 | 1.00 | 73.27 | C |
| ATOM | 571 | O | PRO | A | 90 | 5.542 | −55.499 | −1.017 | 1.00 | 73.27 | O |
| ATOM | 572 | N | GLU | A | 91 | 4.254 | −55.546 | 0.838 | 1.00 | 73.71 | N |
| ATOM | 573 | CA | GLU | A | 91 | 4.167 | −54.094 | 0.976 | 1.00 | 74.09 | C |
| ATOM | 574 | CB | GLU | A | 91 | 3.095 | −53.714 | 2.008 | 1.00 | 74.13 | C |
| ATOM | 575 | CG | GLU | A | 91 | 1.660 | −53.941 | 1.538 | 1.00 | 74.32 | C |
| ATOM | 576 | CD | GLU | A | 91 | 1.490 | −53.220 | −0.578 | 0.00 | 50.00 | C |
| ATOM | 577 | OE1 | GLU | A | 91 | 0.483 | −53.953 | −0.666 | 0.00 | 50.00 | O |
| ATOM | 578 | OE2 | GLU | A | 91 | 2.188 | −52.915 | −1.575 | 0.00 | 50.00 | O |
| ATOM | 579 | C | GLU | A | 91 | 5.529 | −53.526 | 1.387 | 1.00 | 74.25 | C |
| ATOM | 580 | O | GLU | A | 91 | 6.255 | −54.168 | 2.151 | 1.00 | 74.34 | O |
| ATOM | 581 | N | PRO | A | 92 | 5.874 | −52.323 | 0.876 | 1.00 | 74.34 | N |
| ATOM | 582 | CA | PRO | A | 92 | 7.138 | −51.615 | 1.101 | 1.00 | 74.34 | C |
| ATOM | 583 | CB | PRO | A | 92 | 6.734 | −50.133 | 0.987 | 1.00 | 74.35 | C |
| ATOM | 584 | CG | PRO | A | 92 | 5.317 | −50.122 | 0.401 | 1.00 | 74.33 | C |
| ATOM | 585 | CD | PRO | A | 92 | 4.987 | −51.535 | 0.006 | 1.00 | 74.39 | C |
| ATOM | 586 | C | PRO | A | 92 | 7.772 | −51.884 | 2.471 | 1.00 | 74.33 | C |
| ATOM | 587 | O | PRO | A | 92 | 7.109 | −51.737 | 3.508 | 1.00 | 74.31 | O |
| ATOM | 588 | N | THR | A | 93 | 9.052 | −52.260 | 2.465 | 1.00 | 74.15 | N |
| ATOM | 589 | CA | THR | A | 93 | 9.754 | −52.645 | 3.695 | 1.00 | 73.95 | C |
| ATOM | 590 | CB | THR | A | 93 | 10.189 | −54.133 | 3.657 | 1.00 | 74.06 | C |
| ATOM | 591 | OG1 | THR | A | 93 | 10.591 | −54.490 | 2.311 | 1.00 | 73.87 | O |
| ATOM | 592 | CG2 | THR | A | 93 | 9.031 | −55.058 | 4.114 | 1.00 | 74.23 | C |
| ATOM | 593 | C | THR | A | 93 | 10.968 | −51.770 | 4.024 | 1.00 | 73.59 | C |
| ATOM | 594 | O | THR | A | 93 | 10.972 | −51.057 | 5.034 | 1.00 | 73.58 | O |
| ATOM | 595 | N | THR | A | 94 | 11.993 | −51.839 | 3.172 | 1.00 | 73.13 | N |
| ATOM | 596 | CA | THR | A | 94 | 13.241 | −51.097 | 3.381 | 1.00 | 72.66 | C |
| ATOM | 597 | CB | THR | A | 94 | 14.380 | −51.644 | 2.474 | 1.00 | 72.80 | C |
| ATOM | 598 | OG1 | THR | A | 94 | 13.974 | −51.598 | 1.095 | 1.00 | 72.65 | O |
| ATOM | 599 | CG2 | THR | A | 94 | 14.720 | −53.088 | 2.846 | 1.00 | 72.95 | C |
| ATOM | 600 | C | THR | A | 94 | 13.058 | −49.593 | 3.137 | 1.00 | 72.18 | C |
| ATOM | 601 | O | THR | A | 94 | 12.441 | −49.188 | 2.150 | 1.00 | 72.21 | O |
| ATOM | 602 | N | ASP | A | 95 | 13.600 | −48.767 | 4.031 | 1.00 | 71.45 | N |
| ATOM | 603 | CA | ASP | A | 95 | 13.381 | −47.320 | 3.956 | 1.00 | 70.73 | C |
| ATOM | 604 | CB | ASP | A | 95 | 12.585 | −46.839 | 5.180 | 1.00 | 70.79 | C |
| ATOM | 605 | CG | ASP | A | 95 | 12.336 | −46.932 | 5.763 | 0.00 | 50.00 | C |
| ATOM | 606 | OD1 | ASP | A | 95 | 12.950 | −47.842 | 6.397 | 0.00 | 50.00 | O |
| ATOM | 607 | OD2 | ASP | A | 95 | 11.658 | −46.098 | 6.418 | 0.00 | 50.00 | O |
| ATOM | 608 | C | ASP | A | 95 | 14.665 | −46.498 | 3.814 | 1.00 | 70.16 | C |
| ATOM | 609 | O | ASP | A | 95 | 14.738 | −45.367 | 4.304 | 1.00 | 70.07 | O |
| ATOM | 610 | N | CYS | A | 96 | 15.665 | −47.052 | 3.131 | 1.00 | 69.46 | N |
| ATOM | 611 | CA | CYS | A | 96 | 16.933 | −46.343 | 2.957 | 1.00 | 68.81 | C |
| ATOM | 612 | CB | CYS | A | 96 | 17.698 | −46.296 | 4.282 | 1.00 | 68.91 | C |
| ATOM | 613 | SG | CYS | A | 96 | 18.497 | −47.853 | 4.720 | 1.00 | 69.45 | S |
| ATOM | 614 | C | CYS | A | 96 | 17.834 | −46.935 | 1.869 | 1.00 | 68.20 | C |
| ATOM | 615 | O | CYS | A | 96 | 17.435 | −47.834 | 1.130 | 1.00 | 68.12 | O |
| ATOM | 616 | N | PHE | A | 97 | 19.053 | −46.408 | 1.785 | 1.00 | 67.48 | N |
| ATOM | 617 | CA | PHE | A | 97 | 20.049 | −46.870 | 0.830 | 1.00 | 66.69 | C |
| ATOM | 618 | CB | PHE | A | 97 | 20.914 | −45.706 | 0.345 | 1.00 | 66.64 | C |
| ATOM | 619 | CG | PHE | A | 97 | 20.172 | −44.689 | −0.467 | 1.00 | 66.32 | C |
| ATOM | 620 | CD1 | PHE | A | 97 | 19.322 | −43.780 | 0.142 | 1.00 | 66.08 | C |
| ATOM | 621 | CE1 | PHE | A | 97 | 18.640 | −42.841 | −0.602 | 1.00 | 65.88 | C |
| ATOM | 622 | CZ | PHE | A | 97 | 18.811 | −42.793 | −1.968 | 1.00 | 66.18 | C |
| ATOM | 623 | CE2 | PHE | A | 97 | 19.662 | −43.688 | −2.588 | 1.00 | 66.18 | C |
| ATOM | 624 | CD2 | PHE | A | 97 | 20.339 | −44.627 | −1.838 | 1.00 | 66.18 | C |
| ATOM | 625 | C | PHE | A | 97 | 20.950 | −47.902 | 1.482 | 1.00 | 66.32 | C |
| ATOM | 626 | O | PHE | A | 97 | 21.532 | −47.657 | 2.538 | 1.00 | 66.17 | O |
| ATOM | 627 | N | VAL | A | 98 | 21.068 | −49.056 | 0.843 | 1.00 | 65.88 | N |
| ATOM | 628 | CA | VAL | A | 98 | 21.934 | −50.108 | 1.335 | 1.00 | 65.43 | C |
| ATOM | 629 | CB | VAL | A | 98 | 21.178 | −51.438 | 1.474 | 1.00 | 65.36 | C |
| ATOM | 630 | CG1 | VAL | A | 98 | 22.070 | −52.488 | 2.108 | 1.00 | 65.18 | C |
| ATOM | 631 | CG2 | VAL | A | 98 | 19.913 | −51.243 | 2.288 | 1.00 | 65.13 | C |
| ATOM | 632 | C | VAL | A | 98 | 23.090 | −50.287 | 0.368 | 1.00 | 65.26 | C |
| ATOM | 633 | O | VAL | A | 98 | 22.889 | −50.620 | −0.797 | 1.00 | 65.27 | O |
| ATOM | 634 | N | ALA | A | 99 | 24.301 | −50.050 | 0.849 | 1.00 | 65.07 | N |
| ATOM | 635 | CA | ALA | A | 99 | 25.478 | −50.225 | 0.022 | 1.00 | 65.02 | C |
| ATOM | 636 | CB | ALA | A | 99 | 26.521 | −49.179 | 0.359 | 1.00 | 65.00 | C |
| ATOM | 637 | C | ALA | A | 99 | 26.039 | −51.619 | 0.225 | 1.00 | 65.03 | C |
| ATOM | 638 | O | ALA | A | 99 | 26.838 | −51.847 | 1.129 | 1.00 | 65.09 | O |
| ATOM | 639 | N | VAL | A | 100 | 25.609 | −52.555 | −0.609 | 1.00 | 65.12 | N |
| ATOM | 640 | CA | VAL | A | 100 | 26.105 | −53.922 | −0.520 | 1.00 | 65.24 | C |
| ATOM | 641 | CB | VAL | A | 100 | 25.193 | −54.914 | −1.267 | 1.00 | 65.20 | C |
| ATOM | 642 | CG1 | VAL | A | 100 | 23.733 | −54.672 | −0.914 | 1.00 | 65.10 | C |
| ATOM | 643 | CG2 | VAL | A | 100 | 25.600 | −56.345 | −0.952 | 1.00 | 65.15 | C |
| ATOM | 644 | C | VAL | A | 100 | 27.512 | −53.995 | −1.097 | 1.00 | 65.39 | C |
| ATOM | 645 | O | VAL | A | 100 | 27.699 | −53.938 | −2.313 | 1.00 | 65.48 | O |
| ATOM | 646 | N | MET | A | 101 | 28.501 | −54.107 | −0.218 | 1.00 | 65.56 | N |
| ATOM | 647 | CA | MET | A | 101 | 29.894 | −54.161 | −0.638 | 1.00 | 65.78 | C |
| ATOM | 648 | CB | MET | A | 101 | 30.600 | −52.850 | −0.311 | 1.00 | 65.79 | C |
| ATOM | 649 | CG | MET | A | 101 | 30.252 | −52.294 | 1.058 | 1.00 | 65.81 | C |
| ATOM | 650 | SD | MET | A | 101 | 31.344 | −50.959 | 1.580 | 1.00 | 65.65 | S |
| ATOM | 651 | CE | MET | A | 101 | 31.250 | −49.849 | 0.175 | 1.00 | 65.57 | C |
| ATOM | 652 | C | MET | A | 101 | 30.610 | −55.317 | 0.034 | 1.00 | 66.02 | C |
| ATOM | 653 | O | MET | A | 101 | 30.052 | −55.978 | 0.907 | 1.00 | 66.05 | O |
| ATOM | 654 | N | HIS | A | 102 | 31.850 | −55.559 | −0.369 | 1.00 | 66.39 | N |
| ATOM | 655 | CA | HIS | A | 102 | 32.607 | −56.667 | 0.190 | 1.00 | 66.80 | C |
| ATOM | 656 | CB | HIS | A | 102 | 33.918 | −56.881 | −0.569 | 1.00 | 66.73 | C |
| ATOM | 657 | CG | HIS | A | 102 | 34.629 | −58.142 | −0.189 | 1.00 | 66.79 | C |
| ATOM | 658 | ND1 | HIS | A | 102 | 34.164 | −59.392 | −0.540 | 1.00 | 66.68 | N |
| ATOM | 659 | CE1 | HIS | A | 102 | 34.983 | −60.313 | −0.064 | 1.00 | 66.49 | C |
| ATOM | 660 | NE2 | HIS | A | 102 | 35.961 | −59.706 | 0.586 | 1.00 | 66.63 | N |
| ATOM | 661 | CD2 | HIS | A | 102 | 35.762 | −58.347 | 0.525 | 1.00 | 66.78 | C |
| ATOM | 662 | C | HIS | A | 102 | 32.873 | −56.459 | 1.678 | 1.00 | 67.13 | C |
| ATOM | 663 | O | HIS | A | 102 | 32.449 | −55.465 | 2.262 | 1.00 | 67.23 | O |
| ATOM | 664 | N | GLY | A | 103 | 33.572 | −57.408 | 2.285 | 1.00 | 67.46 | N |
| ATOM | 665 | CA | GLY | A | 103 | 33.879 | −57.341 | 3.700 | 1.00 | 67.99 | C |
| ATOM | 666 | C | GLY | A | 103 | 34.212 | −58.729 | 4.186 | 1.00 | 68.39 | C |
| ATOM | 667 | O | GLY | A | 103 | 33.727 | −59.715 | 3.636 | 1.00 | 68.41 | O |
| ATOM | 668 | N | GLU | A | 104 | 35.045 | −58.812 | 5.216 | 1.00 | 68.82 | N |
| ATOM | 669 | CA | GLU | A | 104 | 35.493 | −60.105 | 5.708 | 1.00 | 69.31 | C |
| ATOM | 670 | CB | GLU | A | 104 | 36.829 | −59.968 | 6.450 | 1.00 | 69.45 | C |
| ATOM | 671 | CG | GLU | A | 104 | 37.433 | −61.291 | 6.923 | 1.00 | 70.31 | C |
| ATOM | 672 | CD | GLU | A | 104 | 37.326 | −62.411 | 5.885 | 1.00 | 71.40 | C |
| ATOM | 673 | OE1 | GLU | A | 104 | 36.942 | −62.136 | 4.723 | 1.00 | 71.64 | O |
| ATOM | 674 | OE2 | GLU | A | 104 | 37.624 | −63.575 | 6.240 | 1.00 | 71.78 | O |
| ATOM | 675 | C | GLU | A | 104 | 34.434 | −60.790 | 6.577 | 1.00 | 69.42 | C |
| ATOM | 676 | O | GLU | A | 104 | 34.245 | −62.007 | 6.501 | 1.00 | 69.49 | O |
| ATOM | 677 | N | THR | A | 105 | 33.737 | −60.003 | 7.389 | 1.00 | 69.53 | N |
| ATOM | 678 | CA | THR | A | 105 | 32.691 | −60.542 | 8.248 | 1.00 | 69.56 | C |
| ATOM | 679 | CB | THR | A | 105 | 32.845 | −60.049 | 9.707 | 1.00 | 69.61 | C |
| ATOM | 680 | OG1 | THR | A | 105 | 32.017 | −60.837 | 10.572 | 1.00 | 69.65 | O |
| ATOM | 681 | CG2 | THR | A | 105 | 32.457 | −58.573 | 9.830 | 1.00 | 69.72 | C |
| ATOM | 682 | C | THR | A | 105 | 31.305 | −60.172 | 7.723 | 1.00 | 69.52 | C |
| ATOM | 683 | O | THR | A | 105 | 31.111 | −59.099 | 7.147 | 1.00 | 69.53 | O |
| ATOM | 684 | N | GLU | A | 106 | 30.349 | −61.075 | 7.909 | 1.00 | 69.46 | N |
| ATOM | 685 | CA | GLU | A | 106 | 28.960 | −60.778 | 7.594 | 1.00 | 69.46 | C |
| ATOM | 686 | CB | GLU | A | 106 | 28.137 | −62.066 | 7.517 | 1.00 | 69.51 | C |
| ATOM | 687 | CG | GLU | A | 106 | 28.455 | −62.949 | 6.315 | 1.00 | 69.53 | C |
| ATOM | 688 | CD | GLU | A | 106 | 27.789 | −64.318 | 6.394 | 1.00 | 69.39 | C |
| ATOM | 689 | OE1 | GLU | A | 106 | 27.490 | −64.754 | 7.573 | 1.00 | 69.37 | O |
| ATOM | 690 | OE2 | GLU | A | 106 | 27.562 | −64.963 | 5.282 | 1.00 | 68.93 | O |
| ATOM | 691 | C | GLU | A | 106 | 28.399 | −59.863 | 8.674 | 1.00 | 69.46 | C |
| ATOM | 692 | O | GLU | A | 106 | 28.645 | −60.073 | 9.860 | 1.00 | 69.46 | O |
| ATOM | 693 | N | GLY | A | 107 | 27.650 | −58.845 | 8.265 | 1.00 | 69.47 | N |
| ATOM | 694 | CA | GLY | A | 107 | 27.084 | −57.896 | 9.216 | 1.00 | 69.50 | C |
| ATOM | 695 | C | GLY | A | 107 | 26.766 | −56.551 | 8.593 | 1.00 | 69.52 | C |
| ATOM | 696 | O | GLY | A | 107 | 26.771 | −56.407 | 7.370 | 1.00 | 69.51 | O |
| ATOM | 697 | N | THR | A | 108 | 26.489 | −55.562 | 9.439 | 1.00 | 69.51 | N |
| ATOM | 698 | CA | THR | A | 108 | 26.095 | −54.240 | 8.965 | 1.00 | 69.49 | C |
| ATOM | 699 | CB | THR | A | 108 | 24.573 | −54.020 | 9.092 | 1.00 | 69.48 | C |
| ATOM | 700 | OG1 | THR | A | 108 | 24.213 | −53.964 | 10.478 | 1.00 | 69.55 | O |
| ATOM | 701 | CG2 | THR | A | 108 | 23.798 | −55.139 | 8.407 | 1.00 | 69.46 | C |
| ATOM | 702 | C | THR | A | 108 | 26.787 | −53.116 | 9.725 | 1.00 | 69.49 | C |
| ATOM | 703 | O | THR | A | 108 | 27.509 | −53.354 | 10.695 | 1.00 | 69.48 | O |
| ATOM | 704 | N | VAL | A | 109 | 26.546 | −51.890 | 9.267 | 1.00 | 69.49 | N |
| ATOM | 705 | CA | VAL | A | 109 | 27.063 | −50.680 | 9.901 | 1.00 | 69.46 | C |
| ATOM | 706 | CB | VAL | A | 109 | 28.614 | −50.660 | 9.921 | 1.00 | 69.47 | C |
| ATOM | 707 | CG1 | VAL | A | 109 | 29.132 | −49.424 | 10.648 | 1.00 | 69.54 | C |
| ATOM | 708 | CG2 | VAL | A | 109 | 29.180 | −50.750 | 8.506 | 1.00 | 69.55 | C |
| ATOM | 709 | C | VAL | A | 109 | 26.507 | −49.450 | 9.173 | 1.00 | 69.39 | C |
| ATOM | 710 | O | VAL | A | 109 | 26.653 | −49.325 | 7.956 | 1.00 | 69.42 | O |
| ATOM | 711 | N | PRO | A | 110 | 25.843 | −48.548 | 9.916 | 1.00 | 69.28 | N |
| ATOM | 712 | CA | PRO | A | 110 | 25.193 | −47.376 | 9.325 | 1.00 | 69.20 | C |
| ATOM | 713 | CB | PRO | A | 110 | 24.058 | −47.076 | 10.311 | 1.00 | 69.20 | C |
| ATOM | 714 | CG | PRO | A | 110 | 24.501 | −47.667 | 11.623 | 1.00 | 69.19 | C |
| ATOM | 715 | CD | PRO | A | 110 | 25.653 | −48.608 | 11.376 | 1.00 | 69.25 | C |
| ATOM | 716 | C | PRO | A | 110 | 26.098 | −46.150 | 9.166 | 1.00 | 69.09 | C |
| ATOM | 717 | O | PRO | A | 110 | 25.916 | −45.155 | 9.869 | 1.00 | 69.13 | O |
| ATOM | 718 | N | GLY | A | 111 | 27.048 | −46.216 | 8.237 | 1.00 | 68.96 | N |
| ATOM | 719 | CA | GLY | A | 111 | 27.942 | −45.089 | 7.969 | 1.00 | 68.85 | C |
| ATOM | 720 | C | GLY | A | 111 | 28.984 | −44.872 | 9.058 | 1.00 | 68.81 | C |
| ATOM | 721 | O | GLY | A | 111 | 28.844 | −43.965 | 9.917 | 1.00 | 68.65 | O |
| ATOM | 722 | N | ASN | A | 130 | 26.659 | −31.270 | 8.342 | 1.00 | 66.96 | N |
| ATOM | 723 | CA | ASN | A | 130 | 25.386 | −30.849 | 7.759 | 1.00 | 67.00 | C |
| ATOM | 724 | CB | ASN | A | 130 | 25.594 | −29.640 | 6.836 | 1.00 | 66.96 | C |
| ATOM | 725 | CG | ASN | A | 130 | 24.279 | −29.020 | 6.381 | 1.00 | 66.90 | C |
| ATOM | 726 | OD1 | ASN | A | 130 | 23.843 | −28.004 | 6.943 | 1.00 | 66.78 | O |
| ATOM | 727 | ND2 | ASN | A | 130 | 23.636 | −29.635 | 5.364 | 1.00 | 66.61 | N |
| ATOM | 728 | C | ASN | A | 130 | 24.650 | −31.973 | 7.012 | 1.00 | 67.05 | C |
| ATOM | 729 | O | ASN | A | 130 | 23.429 | −32.106 | 7.132 | 1.00 | 67.08 | O |
| ATOM | 730 | N | THR | A | 131 | 25.399 | −32.772 | 6.249 | 1.00 | 67.04 | N |
| ATOM | 731 | CA | THR | A | 131 | 24.833 | −33.856 | 5.424 | 1.00 | 66.97 | C |
| ATOM | 732 | CB | THR | A | 131 | 25.900 | −34.450 | 4.459 | 1.00 | 66.99 | C |
| ATOM | 733 | OG1 | THR | A | 131 | 25.543 | −35.792 | 4.101 | 1.00 | 66.77 | O |
| ATOM | 734 | CG2 | THR | A | 131 | 27.277 | −34.460 | 5.115 | 1.00 | 67.17 | C |
| ATOM | 735 | C | THR | A | 131 | 24.178 | −34.980 | 6.243 | 1.00 | 66.94 | C |
| ATOM | 736 | O | THR | A | 131 | 24.347 | −35.054 | 7.465 | 1.00 | 66.93 | O |
| ATOM | 737 | N | PHE | A | 132 | 23.431 | −35.851 | 5.561 | 1.00 | 66.81 | N |
| ATOM | 738 | CA | PHE | A | 132 | 22.674 | −36.912 | 6.232 | 1.00 | 66.68 | C |
| ATOM | 739 | CB | PHE | A | 132 | 21.172 | −36.602 | 6.205 | 1.00 | 66.71 | C |
| ATOM | 740 | CG | PHE | A | 132 | 20.815 | −35.278 | 6.826 | 1.00 | 66.79 | C |
| ATOM | 741 | CD1 | PHE | A | 132 | 20.191 | −34.290 | 6.077 | 1.00 | 66.90 | C |
| ATOM | 742 | CE1 | PHE | A | 132 | 19.863 | −33.064 | 6.649 | 1.00 | 67.27 | C |
| ATOM | 743 | CZ | PHE | A | 132 | 20.166 | −32.814 | 7.982 | 1.00 | 67.02 | C |
| ATOM | 744 | CE2 | PHE | A | 132 | 20.793 | −33.791 | 8.737 | 1.00 | 67.01 | C |
| ATOM | 745 | CD2 | PHE | A | 132 | 21.116 | −35.015 | 8.158 | 1.00 | 66.90 | C |
| ATOM | 746 | C | PHE | A | 132 | 22.947 | −38.316 | 5.680 | 1.00 | 66.58 | C |
| ATOM | 747 | O | PHE | A | 132 | 22.646 | −38.620 | 4.521 | 1.00 | 66.52 | O |
| ATOM | 748 | N | LEU | A | 133 | 23.516 | −39.165 | 6.534 | 1.00 | 66.41 | N |
| ATOM | 749 | CA | LEU | A | 133 | 23.870 | −40.539 | 6.181 | 1.00 | 66.14 | C |
| ATOM | 750 | CB | LEU | A | 133 | 25.374 | −40.769 | 6.373 | 1.00 | 66.19 | C |
| ATOM | 751 | CG | LEU | A | 133 | 26.382 | −40.435 | 5.265 | 1.00 | 66.22 | C |
| ATOM | 752 | CD1 | LEU | A | 133 | 26.089 | −39.104 | 4.584 | 1.00 | 66.32 | C |
| ATOM | 753 | CD2 | LEU | A | 133 | 27.804 | −40.457 | 5.821 | 1.00 | 66.13 | C |
| ATOM | 754 | C | LEU | A | 133 | 23.101 | −41.546 | 7.032 | 1.00 | 65.93 | C |
| ATOM | 755 | O | LEU | A | 133 | 23.329 | −42.751 | 6.937 | 1.00 | 65.82 | O |
| ATOM | 756 | N | ASN | A | 134 | 22.205 | −41.051 | 7.882 | 1.00 | 65.76 | N |
| ATOM | 757 | CA | ASN | A | 134 | 21.409 | −41.930 | 8.732 | 1.00 | 65.55 | C |
| ATOM | 758 | CB | ASN | A | 134 | 20.734 | −41.148 | 9.863 | 1.00 | 65.55 | C |
| ATOM | 759 | CG | ASN | A | 134 | 20.140 | −42.057 | 10.930 | 1.00 | 65.49 | C |
| ATOM | 760 | OD1 | ASN | A | 134 | 19.314 | −41.627 | 11.736 | 1.00 | 65.41 | O |
| ATOM | 761 | ND2 | ASN | A | 134 | 20.560 | −43.319 | 10.938 | 1.00 | 65.26 | N |
| ATOM | 762 | C | ASN | A | 134 | 20.373 | −42.672 | 7.900 | 1.00 | 65.39 | C |
| ATOM | 763 | O | ASN | A | 134 | 19.824 | −43.690 | 8.326 | 1.00 | 65.43 | O |
| ATOM | 764 | N | ARG | A | 135 | 20.112 | −42.146 | 6.707 | 1.00 | 65.09 | N |
| ATOM | 765 | CA | ARG | A | 135 | 19.287 | −42.836 | 5.734 | 1.00 | 64.81 | C |
| ATOM | 766 | CB | ARG | A | 135 | 18.497 | −41.831 | 4.894 | 1.00 | 64.75 | C |
| ATOM | 767 | CG | ARG | A | 135 | 17.760 | −40.714 | 5.123 | 0.00 | 50.00 | C |
| ATOM | 768 | CD | ARG | A | 135 | 17.421 | −39.538 | 4.310 | 0.00 | 50.00 | C |
| ATOM | 769 | NE | ARG | A | 135 | 18.323 | −38.615 | 3.780 | 0.00 | 50.00 | N |
| ATOM | 770 | CZ | ARG | A | 135 | 18.391 | −37.354 | 3.482 | 0.00 | 50.00 | C |
| ATOM | 771 | NH1 | ARG | A | 135 | 19.376 | −36.980 | 2.625 | 0.00 | 50.00 | N |
| ATOM | 772 | NH2 | ARG | A | 135 | 17.696 | −36.373 | 4.013 | 0.00 | 50.00 | N |
| ATOM | 773 | C | ARG | A | 135 | 20.213 | −43.669 | 4.857 | 1.00 | 64.65 | C |
| ATOM | 774 | O | ARG | A | 135 | 19.912 | −43.944 | 3.695 | 1.00 | 64.66 | O |
| ATOM | 775 | N | PHE | A | 136 | 21.344 | −44.072 | 5.431 | 1.00 | 64.44 | N |
| ATOM | 776 | CA | PHE | A | 136 | 22.377 | −44.795 | 4.695 | 1.00 | 64.19 | C |
| ATOM | 777 | CB | PHE | A | 136 | 23.496 | −43.832 | 4.295 | 1.00 | 64.24 | C |
| ATOM | 778 | CG | PHE | A | 136 | 24.064 | −44.089 | 2.932 | 1.00 | 64.14 | C |
| ATOM | 779 | CD1 | PHE | A | 136 | 23.904 | −43.155 | 1.919 | 1.00 | 64.00 | C |
| ATOM | 780 | CE1 | PHE | A | 136 | 24.424 | −43.377 | 0.663 | 1.00 | 64.15 | C |
| ATOM | 781 | CZ | PHE | A | 136 | 25.111 | −44.548 | 0.403 | 1.00 | 64.46 | C |
| ATOM | 782 | CE2 | PHE | A | 136 | 25.277 | −45.491 | 1.407 | 1.00 | 64.42 | C |
| ATOM | 783 | CD2 | PHE | A | 136 | 24.756 | −45.257 | 2.662 | 1.00 | 64.17 | C |
| ATOM | 784 | C | PHE | A | 136 | 22.959 | −45.939 | 5.525 | 1.00 | 63.99 | C |
| ATOM | 785 | O | PHE | A | 136 | 23.550 | −45.718 | 6.586 | 1.00 | 63.97 | O |
| ATOM | 786 | N | MET | A | 137 | 22.793 | −47.160 | 5.028 | 1.00 | 63.68 | N |
| ATOM | 787 | CA | MET | A | 137 | 23.301 | −48.347 | 5.703 | 1.00 | 63.43 | C |
| ATOM | 788 | CB | MET | A | 137 | 22.165 | −49.343 | 5.945 | 1.00 | 63.62 | C |
| ATOM | 789 | CG | MET | A | 137 | 21.184 | −48.929 | 7.033 | 1.00 | 64.35 | C |
| ATOM | 790 | SD | MET | A | 137 | 21.680 | −49.495 | 8.676 | 1.00 | 66.23 | S |
| ATOM | 791 | CE | MET | A | 137 | 21.543 | −51.275 | 8.465 | 1.00 | 65.88 | C |
| ATOM | 792 | C | MET | A | 137 | 24.384 | −49.000 | 4.858 | 1.00 | 63.01 | C |
| ATOM | 793 | O | MET | A | 137 | 24.486 | −48.735 | 3.661 | 1.00 | 63.09 | O |
| ATOM | 794 | N | CYS | A | 138 | 25.188 | −49.857 | 5.479 | 1.00 | 62.44 | N |
| ATOM | 795 | CA | CYS | A | 138 | 26.232 | −50.576 | 4.757 | 1.00 | 61.94 | C |
| ATOM | 796 | CB | CYS | A | 138 | 27.595 | −49.918 | 4.975 | 1.00 | 62.03 | C |
| ATOM | 797 | SG | CYS | A | 138 | 28.990 | −50.936 | 4.439 | 1.00 | 62.33 | S |
| ATOM | 798 | C | CYS | A | 138 | 26.295 | −52.041 | 5.163 | 1.00 | 61.48 | C |
| ATOM | 799 | O | CYS | A | 138 | 26.422 | −52.365 | 6.345 | 1.00 | 61.39 | O |
| ATOM | 800 | N | ALA | A | 139 | 26.211 | −52.921 | 4.172 | 1.00 | 60.93 | N |
| ATOM | 801 | CA | ALA | A | 139 | 26.280 | −54.357 | 4.408 | 1.00 | 60.47 | C |
| ATOM | 802 | CB | ALA | A | 139 | 25.044 | −55.046 | 3.856 | 1.00 | 60.46 | C |
| ATOM | 803 | C | ALA | A | 139 | 27.541 | −54.939 | 3.783 | 1.00 | 60.13 | C |
| ATOM | 804 | O | ALA | A | 139 | 27.987 | −54.485 | 2.729 | 1.00 | 60.12 | O |
| ATOM | 805 | N | GLN | A | 140 | 28.113 | −55.942 | 4.441 | 1.00 | 59.68 | N |
| ATOM | 806 | CA | GLN | A | 140 | 29.350 | −56.562 | 3.985 | 1.00 | 59.22 | C |
| ATOM | 807 | CB | GLN | A | 140 | 30.513 | −56.145 | 4.882 | 1.00 | 59.22 | C |
| ATOM | 808 | CG | GLN | A | 140 | 30.723 | −54.656 | 4.993 | 1.00 | 59.09 | C |
| ATOM | 809 | CD | GLN | A | 140 | 31.949 | −54.321 | 5.802 | 1.00 | 59.09 | C |
| ATOM | 810 | OE1 | GLN | A | 140 | 32.772 | −55.192 | 6.084 | 1.00 | 59.44 | O |
| ATOM | 811 | NE2 | GLN | A | 140 | 32.080 | −53.058 | 6.185 | 1.00 | 59.05 | N |
| ATOM | 812 | C | GLN | A | 140 | 29.231 | −58.074 | 4.025 | 1.00 | 58.94 | C |
| ATOM | 813 | O | GLN | A | 140 | 28.591 | −58.624 | 4.920 | 1.00 | 58.94 | O |
| ATOM | 814 | N | LEU | A | 141 | 29.860 | −58.747 | 3.067 | 1.00 | 58.55 | N |
| ATOM | 815 | CA | LEU | A | 141 | 29.887 | −60.207 | 3.081 | 1.00 | 58.30 | C |
| ATOM | 816 | CB | LEU | A | 141 | 28.481 | −60.785 | 2.861 | 1.00 | 58.30 | C |
| ATOM | 817 | CG | LEU | A | 141 | 27.730 | −60.539 | 1.550 | 1.00 | 58.25 | C |
| ATOM | 818 | CD1 | LEU | A | 141 | 26.394 | −61.267 | 1.581 | 1.00 | 58.11 | C |
| ATOM | 819 | CD2 | LEU | A | 141 | 27.524 | −59.056 | 1.278 | 1.00 | 58.38 | C |
| ATOM | 820 | C | LEU | A | 141 | 30.890 | −60.810 | 2.099 | 1.00 | 58.06 | C |
| ATOM | 821 | O | LEU | A | 141 | 30.953 | −60.403 | 0.942 | 1.00 | 58.07 | O |
| ATOM | 822 | N | PRO | A | 142 | 31.681 | −61.788 | 2.571 | 1.00 | 57.87 | N |
| ATOM | 823 | CA | PRO | A | 142 | 32.663 | −62.505 | 1.761 | 1.00 | 57.70 | C |
| ATOM | 824 | CB | PRO | A | 142 | 33.217 | −63.554 | 2.728 | 1.00 | 57.74 | C |
| ATOM | 825 | CG | PRO | A | 142 | 32.219 | −63.635 | 3.840 | 1.00 | 57.78 | C |
| ATOM | 826 | CD | PRO | A | 142 | 31.664 | −62.265 | 3.963 | 1.00 | 57.80 | C |
| ATOM | 827 | C | PRO | A | 142 | 31.999 | −63.187 | 0.578 | 1.00 | 57.59 | C |
| ATOM | 828 | O | PRO | A | 142 | 31.212 | −64.109 | 0.759 | 1.00 | 57.61 | O |
| ATOM | 829 | N | ASN | A | 143 | 32.327 | −62.729 | −0.625 | 1.00 | 57.52 | N |
| ATOM | 830 | CA | ASN | A | 143 | 31.640 | −63.152 | −1.837 | 1.00 | 57.41 | C |
| ATOM | 831 | CB | ASN | A | 143 | 30.167 | −62.745 | −1.756 | 1.00 | 57.34 | C |
| ATOM | 832 | CG | ASN | A | 143 | 29.403 | −63.022 | −3.037 | 1.00 | 57.28 | C |
| ATOM | 833 | OD1 | ASN | A | 143 | 29.960 | −62.987 | −4.136 | 1.00 | 57.32 | O |
| ATOM | 834 | ND2 | ASN | A | 143 | 28.109 | −63.281 | −2.901 | 1.00 | 56.70 | N |
| ATOM | 835 | C | ASN | A | 143 | 32.314 | −62.516 | −3.050 | 1.00 | 57.47 | C |
| ATOM | 836 | O | ASN | A | 143 | 32.443 | −61.295 | −3.124 | 1.00 | 57.41 | O |
| ATOM | 837 | N | GLN | A | 144 | 32.737 | −63.350 | −3.998 | 1.00 | 57.56 | N |
| ATOM | 838 | CA | GLN | A | 144 | 33.563 | −62.908 | −5.129 | 1.00 | 57.72 | C |
| ATOM | 839 | CB | GLN | A | 144 | 33.773 | −64.055 | −6.110 | 1.00 | 57.81 | C |
| ATOM | 840 | CG | GLN | A | 144 | 34.101 | −65.378 | −5.467 | 1.00 | 58.02 | C |
| ATOM | 841 | CD | GLN | A | 144 | 34.468 | −66.419 | −6.497 | 1.00 | 58.57 | C |
| ATOM | 842 | OE1 | GLN | A | 144 | 35.365 | −66.207 | −7.317 | 1.00 | 58.64 | O |
| ATOM | 843 | NE2 | GLN | A | 144 | 33.774 | −67.553 | −6.466 | 1.00 | 58.83 | N |
| ATOM | 844 | C | GLN | A | 144 | 32.996 | −61.713 | −5.891 | 1.00 | 57.73 | C |
| ATOM | 845 | O | GLN | A | 144 | 33.729 | −60.782 | −6.265 | 1.00 | 57.88 | O |
| ATOM | 846 | N | VAL | A | 145 | 31.693 | −61.756 | −6.143 | 1.00 | 57.64 | N |
| ATOM | 847 | CA | VAL | A | 145 | 31.037 | −60.663 | −6.830 | 1.00 | 57.55 | C |
| ATOM | 848 | CB | VAL | A | 145 | 29.515 | −60.834 | −6.849 | 1.00 | 57.60 | C |
| ATOM | 849 | CG1 | VAL | A | 145 | 29.149 | −62.269 | −7.189 | 1.00 | 57.62 | C |
| ATOM | 850 | CG2 | VAL | A | 145 | 28.891 | −59.867 | −7.844 | 1.00 | 57.54 | C |
| ATOM | 851 | C | VAL | A | 145 | 31.396 | −59.369 | −6.123 | 1.00 | 57.47 | C |
| ATOM | 852 | O | VAL | A | 145 | 31.752 | −58.386 | −6.764 | 1.00 | 57.53 | O |
| ATOM | 853 | N | LEU | A | 146 | 31.325 | −59.386 | −4.797 | 1.00 | 57.46 | N |
| ATOM | 854 | CA | LEU | A | 146 | 31.695 | −58.222 | −4.001 | 1.00 | 57.59 | C |
| ATOM | 855 | CB | LEU | A | 146 | 31.218 | −58.367 | −2.553 | 1.00 | 57.59 | C |
| ATOM | 856 | CG | LEU | A | 146 | 29.750 | −58.045 | −2.263 | 1.00 | 57.56 | C |
| ATOM | 857 | CD1 | LEU | A | 146 | 29.270 | −56.879 | −3.119 | 1.00 | 57.23 | C |
| ATOM | 858 | CD2 | LEU | A | 146 | 28.882 | −59.266 | −2.489 | 1.00 | 57.83 | C |
| ATOM | 859 | C | LEU | A | 146 | 33.197 | −57.934 | −4.046 | 1.00 | 57.65 | C |
| ATOM | 860 | O | LEU | A | 146 | 33.635 | −56.840 | −3.698 | 1.00 | 57.62 | O |
| ATOM | 861 | N | GLU | A | 147 | 33.985 | −58.917 | −4.466 | 1.00 | 57.74 | N |
| ATOM | 862 | CA | GLU | A | 147 | 35.396 | −58.681 | −4.719 | 1.00 | 57.96 | C |
| ATOM | 863 | CB | GLU | A | 147 | 36.152 | −59.994 | −4.928 | 1.00 | 57.87 | C |
| ATOM | 864 | CG | GLU | A | 147 | 36.190 | −60.912 | −3.725 | 1.00 | 58.25 | C |
| ATOM | 865 | CD | GLU | A | 147 | 36.827 | −62.253 | −4.048 | 1.00 | 58.44 | C |
| ATOM | 866 | OE1 | GLU | A | 147 | 36.631 | −63.217 | −3.271 | 1.00 | 58.79 | O |
| ATOM | 867 | OE2 | GLU | A | 147 | 37.520 | −62.343 | −5.086 | 1.00 | 59.30 | O |
| ATOM | 868 | C | GLU | A | 147 | 35.520 | −57.827 | −5.969 | 1.00 | 57.91 | C |
| ATOM | 869 | O | GLU | A | 147 | 36.266 | −56.850 | −5.996 | 1.00 | 58.03 | O |
| ATOM | 870 | N | SER | A | 148 | 34.781 | −58.197 | −7.009 | 1.00 | 57.86 | N |
| ATOM | 871 | CA | SER | A | 148 | 34.875 | −57.474 | −8.274 | 1.00 | 57.89 | C |
| ATOM | 872 | CB | SER | A | 148 | 34.270 | −58.296 | −9.409 | 1.00 | 57.92 | C |
| ATOM | 873 | OG | SER | A | 148 | 34.858 | −59.582 | −9.471 | 1.00 | 58.15 | O |
| ATOM | 874 | C | SER | A | 148 | 34.220 | −56.091 | −8.222 | 1.00 | 57.91 | C |
| ATOM | 875 | O | SER | A | 148 | 34.816 | −55.099 | −8.641 | 1.00 | 58.01 | O |
| ATOM | 876 | N | ILE | A | 149 | 32.995 | −56.031 | −7.708 | 1.00 | 57.82 | N |
| ATOM | 877 | CA | ILE | A | 149 | 32.216 | −54.795 | −7.699 | 1.00 | 57.70 | C |
| ATOM | 878 | CB | ILE | A | 149 | 31.161 | −54.802 | −8.818 | 1.00 | 57.63 | C |
| ATOM | 879 | CG1 | ILE | A | 149 | 30.180 | −55.962 | −8.620 | 1.00 | 57.21 | C |
| ATOM | 880 | CD1 | ILE | A | 149 | 29.012 | −55.946 | −9.580 | 1.00 | 56.31 | C |
| ATOM | 881 | CG2 | ILE | A | 149 | 31.825 | −54.901 | −10.175 | 1.00 | 57.78 | C |
| ATOM | 882 | C | ILE | A | 149 | 31.484 | −54.602 | −6.378 | 1.00 | 57.81 | C |
| ATOM | 883 | O | ILE | A | 149 | 31.605 | −55.418 | −5.467 | 1.00 | 58.00 | O |
| ATOM | 884 | N | SER | A | 150 | 30.725 | −53.517 | −6.272 | 1.00 | 57.80 | N |
| ATOM | 885 | CA | SER | A | 150 | 29.808 | −53.345 | −5.146 | 1.00 | 57.87 | C |
| ATOM | 886 | CB | SER | A | 150 | 30.424 | −52.492 | −4.031 | 1.00 | 57.80 | C |
| ATOM | 887 | OG | SER | A | 150 | 31.122 | −51.381 | −4.555 | 1.00 | 58.07 | O |
| ATOM | 888 | C | SER | A | 150 | 28.472 | −52.780 | −5.620 | 1.00 | 57.82 | C |
| ATOM | 889 | O | SER | A | 150 | 28.417 | −51.969 | −6.546 | 1.00 | 57.84 | O |
| ATOM | 890 | N | ILE | A | 151 | 27.393 | −53.233 | −4.994 | 1.00 | 57.75 | N |
| ATOM | 891 | CA | ILE | A | 151 | 26.056 | −52.907 | −5.461 | 1.00 | 57.75 | C |
| ATOM | 892 | CB | ILE | A | 151 | 25.248 | −54.178 | −5.733 | 1.00 | 57.69 | C |
| ATOM | 893 | CG1 | ILE | A | 151 | 26.076 | −55.163 | −6.555 | 1.00 | 57.79 | C |
| ATOM | 894 | CD1 | ILE | A | 151 | 25.580 | −56.585 | −6.471 | 1.00 | 58.36 | C |
| ATOM | 895 | CG2 | ILE | A | 151 | 23.946 | −53.837 | −6.437 | 1.00 | 57.61 | C |
| ATOM | 896 | C | ILE | A | 151 | 25.307 | −52.074 | −4.438 | 1.00 | 57.80 | C |
| ATOM | 897 | O | ILE | A | 151 | 25.108 | −52.502 | −3.302 | 1.00 | 57.77 | O |
| ATOM | 898 | N | ILE | A | 152 | 24.884 | −50.882 | −4.838 | 1.00 | 57.79 | N |
| ATOM | 899 | CA | ILE | A | 152 | 24.129 | −50.046 | −3.928 | 1.00 | 57.80 | C |
| ATOM | 900 | CB | ILE | A | 152 | 24.593 | −48.573 | −3.980 | 1.00 | 57.84 | C |
| ATOM | 901 | CG1 | ILE | A | 152 | 23.502 | −47.669 | −4.549 | 1.00 | 58.21 | C |
| ATOM | 902 | CD1 | ILE | A | 152 | 22.496 | −47.213 | −3.501 | 1.00 | 58.80 | C |
| ATOM | 903 | CG2 | ILE | A | 152 | 25.918 | −48.445 | −4.728 | 1.00 | 57.83 | C |
| ATOM | 904 | C | ILE | A | 152 | 22.633 | −50.189 | −4.214 | 1.00 | 57.75 | C |
| ATOM | 905 | O | ILE | A | 152 | 22.177 | −49.991 | −5.340 | 1.00 | 57.62 | O |
| ATOM | 906 | N | ASP | A | 153 | 21.886 | −50.564 | −3.181 | 1.00 | 57.80 | N |
| ATOM | 907 | CA | ASP | A | 153 | 20.450 | −50.776 | −3.283 | 1.00 | 57.91 | C |
| ATOM | 908 | CB | ASP | A | 153 | 20.048 | −52.005 | −2.470 | 1.00 | 57.99 | C |
| ATOM | 909 | CG | ASP | A | 153 | 18.582 | −52.358 | −2.625 | 1.00 | 58.23 | C |
| ATOM | 910 | OD1 | ASP | A | 153 | 17.831 | −51.554 | −3.219 | 1.00 | 58.41 | O |
| ATOM | 911 | OD2 | ASP | A | 153 | 18.181 | −53.444 | −2.147 | 1.00 | 58.26 | O |
| ATOM | 912 | C | ASP | A | 153 | 19.710 | −49.549 | −2.770 | 1.00 | 58.00 | C |
| ATOM | 913 | O | ASP | A | 153 | 20.084 | −48.975 | −1.746 | 1.00 | 58.18 | O |
| ATOM | 914 | N | THR | A | 154 | 18.652 | −49.160 | −3.475 | 1.00 | 58.02 | N |
| ATOM | 915 | CA | THR | A | 154 | 17.958 | −47.904 | −3.200 | 1.00 | 57.95 | C |
| ATOM | 916 | CB | THR | A | 154 | 17.814 | −47.058 | −4.482 | 1.00 | 57.97 | C |
| ATOM | 917 | OG1 | THR | A | 154 | 17.036 | −47.776 | −5.454 | 1.00 | 57.67 | O |
| ATOM | 918 | CG2 | THR | A | 154 | 19.182 | −46.726 | −5.059 | 1.00 | 57.86 | C |
| ATOM | 919 | C | THR | A | 154 | 16.568 | −48.117 | −2.617 | 1.00 | 58.01 | C |
| ATOM | 920 | O | THR | A | 154 | 16.008 | −49.205 | −2.739 | 1.00 | 57.95 | O |
| ATOM | 921 | N | PRO | A | 155 | 16.009 | −47.071 | −1.977 | 1.00 | 58.12 | N |
| ATOM | 922 | CA | PRO | A | 155 | 14.614 | −47.083 | −1.545 | 1.00 | 58.12 | C |
| ATOM | 923 | CB | PRO | A | 155 | 14.458 | −45.757 | −0.788 | 1.00 | 58.01 | C |
| ATOM | 924 | CG | PRO | A | 155 | 15.838 | −45.284 | −0.511 | 1.00 | 58.04 | C |
| ATOM | 925 | CD | PRO | A | 155 | 16.684 | −45.812 | −1.616 | 1.00 | 58.18 | C |
| ATOM | 926 | C | PRO | A | 155 | 13.682 | −47.093 | −2.747 | 1.00 | 58.22 | C |
| ATOM | 927 | O | PRO | A | 155 | 14.050 | −46.623 | −3.829 | 1.00 | 58.19 | O |
| ATOM | 928 | N | GLY | A | 156 | 12.483 | −47.628 | −2.552 | 1.00 | 58.35 | N |
| ATOM | 929 | CA | GLY | A | 156 | 11.492 | −47.689 | −3.616 | 1.00 | 58.61 | C |
| ATOM | 930 | C | GLY | A | 156 | 10.938 | −46.324 | −3.963 | 1.00 | 58.79 | C |
| ATOM | 931 | O | GLY | A | 156 | 10.545 | −45.557 | −3.083 | 1.00 | 58.87 | O |
| ATOM | 932 | N | ILE | A | 157 | 10.916 | −46.016 | −5.253 | 1.00 | 58.96 | N |
| ATOM | 933 | CA | ILE | A | 157 | 10.340 | −44.769 | −5.713 | 1.00 | 59.18 | C |
| ATOM | 934 | CB | ILE | A | 157 | 10.632 | −44.544 | −7.199 | 1.00 | 59.11 | C |
| ATOM | 935 | CG1 | ILE | A | 157 | 12.137 | −44.645 | −7.447 | 1.00 | 59.27 | C |
| ATOM | 936 | CD1 | ILE | A | 157 | 12.551 | −44.359 | −8.875 | 1.00 | 59.64 | C |
| ATOM | 937 | CG2 | ILE | A | 157 | 10.127 | −43.190 | −7.640 | 1.00 | 59.13 | C |
| ATOM | 938 | C | ILE | A | 157 | 8.838 | −44.792 | −5.459 | 1.00 | 59.40 | C |
| ATOM | 939 | O | ILE | A | 157 | 8.145 | −45.715 | −5.877 | 1.00 | 59.29 | O |
| ATOM | 940 | N | LEU | A | 158 | 8.344 | −43.786 | −4.745 | 1.00 | 59.83 | N |
| ATOM | 941 | CA | LEU | A | 158 | 6.924 | −43.723 | −4.408 | 1.00 | 60.23 | C |
| ATOM | 942 | CB | LEU | A | 158 | 6.614 | −42.471 | −3.580 | 1.00 | 60.13 | C |
| ATOM | 943 | CG | LEU | A | 158 | 6.013 | −41.410 | −3.969 | 0.00 | 50.00 | C |
| ATOM | 944 | CD1 | LEU | A | 158 | 5.164 | −41.219 | −5.172 | 0.00 | 50.00 | C |
| ATOM | 945 | CD2 | LEU | A | 158 | 5.392 | −40.676 | −2.774 | 0.00 | 50.00 | C |
| ATOM | 946 | C | LEU | A | 158 | 6.063 | −43.761 | −5.668 | 1.00 | 60.49 | C |
| ATOM | 947 | O | LEU | A | 158 | 6.377 | −43.110 | −6.666 | 1.00 | 60.52 | O |
| ATOM | 948 | N | SER | A | 159 | 4.984 | −44.535 | −5.618 | 1.00 | 60.79 | N |
| ATOM | 949 | CA | SER | A | 159 | 4.070 | −44.651 | −6.748 | 1.00 | 61.07 | C |
| ATOM | 950 | CB | SER | A | 159 | 3.135 | −45.849 | −6.561 | 1.00 | 61.20 | C |
| ATOM | 951 | OG | SER | A | 159 | 2.477 | −46.173 | −7.774 | 1.00 | 61.72 | O |
| ATOM | 952 | C | SER | A | 159 | 3.260 | −43.364 | −6.942 | 1.00 | 61.06 | C |
| ATOM | 953 | O | SER | A | 159 | 2.043 | −43.338 | −6.733 | 1.00 | 61.02 | O |
| ATOM | 954 | N | ARG | A | 167 | 8.576 | −37.115 | 1.268 | 1.00 | 65.73 | N |
| ATOM | 955 | CA | ARG | A | 167 | 9.108 | −35.811 | 1.654 | 1.00 | 65.83 | C |
| ATOM | 956 | CB | ARG | A | 167 | 8.000 | −34.902 | 2.220 | 1.00 | 65.92 | C |
| ATOM | 957 | CG | ARG | A | 167 | 6.850 | −34.591 | 1.263 | 1.00 | 66.25 | C |
| ATOM | 958 | CD | ARG | A | 167 | 5.617 | −35.448 | 1.553 | 1.00 | 66.72 | C |
| ATOM | 959 | NE | ARG | A | 167 | 5.726 | −36.803 | 1.001 | 1.00 | 67.11 | N |
| ATOM | 960 | CZ | ARG | A | 167 | 4.818 | −37.760 | 1.181 | 1.00 | 67.11 | C |
| ATOM | 961 | NH1 | ARG | A | 167 | 3.728 | −37.514 | 1.905 | 1.00 | 67.24 | N |
| ATOM | 962 | NH2 | ARG | A | 167 | 4.998 | −38.961 | 0.640 | 1.00 | 66.65 | N |
| ATOM | 963 | C | ARG | A | 167 | 10.217 | −35.961 | 2.690 | 1.00 | 65.75 | C |
| ATOM | 964 | O | ARG | A | 167 | 10.033 | −35.617 | 3.858 | 1.00 | 65.80 | O |
| ATOM | 965 | N | GLY | A | 168 | 11.367 | −36.474 | 2.266 | 1.00 | 65.67 | N |
| ATOM | 966 | CA | GLY | A | 168 | 12.498 | −36.639 | 3.172 | 1.00 | 65.55 | C |
| ATOM | 967 | C | GLY | A | 168 | 13.752 | −35.936 | 2.691 | 1.00 | 65.49 | C |
| ATOM | 968 | O | GLY | A | 168 | 14.343 | −35.132 | 3.410 | 1.00 | 65.38 | O |
| ATOM | 969 | N | TYR | A | 169 | 14.151 | −36.239 | 1.462 | 1.00 | 65.52 | N |
| ATOM | 970 | CA | TYR | A | 169 | 15.392 | −35.720 | 0.898 | 1.00 | 65.52 | C |
| ATOM | 971 | CB | TYR | A | 169 | 16.520 | −36.744 | 1.084 | 1.00 | 65.60 | C |
| ATOM | 972 | CG | TYR | A | 169 | 16.060 | −38.185 | 0.979 | 1.00 | 65.63 | C |
| ATOM | 973 | CD1 | TYR | A | 169 | 15.809 | −38.940 | 2.121 | 1.00 | 65.74 | C |
| ATOM | 974 | CE1 | TYR | A | 169 | 15.382 | −40.256 | 2.032 | 1.00 | 65.83 | C |
| ATOM | 975 | CZ | TYR | A | 169 | 15.198 | −40.834 | 0.790 | 1.00 | 65.80 | C |
| ATOM | 976 | OH | TYR | A | 169 | 14.772 | −42.140 | 0.699 | 1.00 | 65.64 | O |
| ATOM | 977 | CE2 | TYR | A | 169 | 15.438 | −40.105 | −0.360 | 1.00 | 65.77 | C |
| ATOM | 978 | CD2 | TYR | A | 169 | 15.866 | −38.788 | −0.260 | 1.00 | 65.73 | C |
| ATOM | 979 | C | TYR | A | 169 | 15.216 | −35.383 | −0.585 | 1.00 | 65.44 | C |
| ATOM | 980 | O | TYR | A | 169 | 14.107 | −35.459 | −1.118 | 1.00 | 65.44 | O |
| ATOM | 981 | N | ASP | A | 170 | 16.312 | −35.007 | −1.242 | 1.00 | 65.30 | N |
| ATOM | 982 | CA | ASP | A | 170 | 16.294 | −34.711 | −2.674 | 1.00 | 65.09 | C |
| ATOM | 983 | CB | ASP | A | 170 | 17.247 | −33.557 | −2.999 | 1.00 | 65.12 | C |
| ATOM | 984 | CG | ASP | A | 170 | 16.898 | −32.862 | −4.304 | 1.00 | 65.25 | C |
| ATOM | 985 | OD1 | ASP | A | 170 | 17.312 | −31.695 | −4.484 | 1.00 | 65.06 | O |
| ATOM | 986 | OD2 | ASP | A | 170 | 16.211 | −33.481 | −5.149 | 1.00 | 65.55 | O |
| ATOM | 987 | C | ASP | A | 170 | 16.637 | −35.965 | −3.486 | 1.00 | 64.95 | C |
| ATOM | 988 | O | ASP | A | 170 | 17.698 | −36.058 | −4.131 | 1.00 | 64.78 | O |
| ATOM | 989 | N | PHE | A | 171 | 15.719 | −36.926 | −3.442 | 1.00 | 64.77 | N |
| ATOM | 990 | CA | PHE | A | 171 | 15.907 | −38.217 | −4.088 | 1.00 | 64.63 | C |
| ATOM | 991 | CB | PHE | A | 171 | 14.580 | −38.975 | −4.165 | 1.00 | 64.63 | C |
| ATOM | 992 | CG | PHE | A | 171 | 14.728 | −40.414 | −4.560 | 1.00 | 64.58 | C |
| ATOM | 993 | CD1 | PHE | A | 171 | 15.480 | −41.283 | −3.788 | 1.00 | 64.37 | C |
| ATOM | 994 | CE1 | PHE | A | 171 | 15.616 | −42.608 | −4.146 | 1.00 | 64.43 | C |
| ATOM | 995 | CZ | PHE | A | 171 | 14.992 | −43.083 | −5.285 | 1.00 | 64.73 | C |
| ATOM | 996 | CE2 | PHE | A | 171 | 14.237 | −42.228 | −6.062 | 1.00 | 64.67 | C |
| ATOM | 997 | CD2 | PHE | A | 171 | 14.107 | −40.902 | −5.697 | 1.00 | 64.81 | C |
| ATOM | 998 | C | PHE | A | 171 | 16.534 | −38.064 | −5.470 | 1.00 | 64.56 | C |
| ATOM | 999 | O | PHE | A | 171 | 17.609 | −38.616 | −5.734 | 1.00 | 64.43 | O |
| ATOM | 1000 | N | PRO | A | 172 | 15.872 | −37.306 | −6.357 | 1.00 | 64.52 | N |
| ATOM | 1001 | CA | PRO | A | 172 | 16.455 | −37.032 | −7.663 | 1.00 | 64.49 | C |
| ATOM | 1002 | CB | PRO | A | 172 | 15.573 | −35.907 | −8.202 | 1.00 | 64.54 | C |
| ATOM | 1003 | CG | PRO | A | 172 | 14.246 | −36.174 | −7.585 | 1.00 | 64.64 | C |
| ATOM | 1004 | CD | PRO | A | 172 | 14.547 | −36.678 | −6.203 | 1.00 | 64.57 | C |
| ATOM | 1005 | C | PRO | A | 172 | 17.916 | −36.583 | −7.571 | 1.00 | 64.39 | C |
| ATOM | 1006 | O | PRO | A | 172 | 18.775 | −37.148 | −8.258 | 1.00 | 64.27 | O |
| ATOM | 1007 | N | ALA | A | 173 | 18.200 | −35.592 | −6.729 | 1.00 | 64.18 | N |
| ATOM | 1008 | CA | ALA | A | 173 | 19.568 | −35.093 | −6.596 | 1.00 | 64.15 | C |
| ATOM | 1009 | CB | ALA | A | 173 | 19.641 | −33.959 | −5.592 | 1.00 | 64.18 | C |
| ATOM | 1010 | C | ALA | A | 173 | 20.547 | −36.206 | −6.226 | 1.00 | 64.15 | C |
| ATOM | 1011 | O | ALA | A | 173 | 21.636 | −36.305 | −6.809 | 1.00 | 64.04 | O |
| ATOM | 1012 | N | VAL | A | 174 | 20.168 | −37.051 | −5.270 | 1.00 | 64.23 | N |
| ATOM | 1013 | CA | VAL | A | 174 | 21.045 | −38.177 | −4.922 | 1.00 | 64.12 | C |
| ATOM | 1014 | CB | VAL | A | 174 | 20.627 | −38.899 | −3.608 | 1.00 | 64.14 | C |
| ATOM | 1015 | CG1 | VAL | A | 174 | 19.189 | −38.598 | −3.246 | 1.00 | 64.11 | C |
| ATOM | 1016 | CG2 | VAL | A | 174 | 20.874 | −40.397 | −3.705 | 1.00 | 64.15 | C |
| ATOM | 1017 | C | VAL | A | 174 | 21.219 | −39.162 | −6.092 | 1.00 | 64.08 | C |
| ATOM | 1018 | O | VAL | A | 174 | 22.340 | −39.617 | −6.378 | 1.00 | 63.98 | O |
| ATOM | 1019 | N | LEU | A | 175 | 20.121 | −39.466 | −6.781 | 1.00 | 64.02 | N |
| ATOM | 1020 | CA | LEU | A | 175 | 20.200 | −40.298 | −7.976 | 1.00 | 63.80 | C |
| ATOM | 1021 | CB | LEU | A | 175 | 18.813 | −40.556 | −8.557 | 1.00 | 63.79 | C |
| ATOM | 1022 | CG | LEU | A | 175 | 18.168 | −41.866 | −8.103 | 1.00 | 64.02 | C |
| ATOM | 1023 | CD1 | LEU | A | 175 | 16.760 | −42.013 | −8.663 | 1.00 | 64.02 | C |
| ATOM | 1024 | CD2 | LEU | A | 175 | 19.034 | −43.050 | −8.512 | 1.00 | 64.22 | C |
| ATOM | 1025 | C | LEU | A | 175 | 21.107 | −39.679 | −9.034 | 1.00 | 63.78 | C |
| ATOM | 1026 | O | LEU | A | 175 | 21.733 | −40.386 | −9.820 | 1.00 | 63.66 | O |
| ATOM | 1027 | N | ARG | A | 176 | 21.181 | −38.355 | −9.049 | 1.00 | 63.87 | N |
| ATOM | 1028 | CA | ARG | A | 176 | 22.050 | −37.663 | −9.989 | 1.00 | 63.82 | C |
| ATOM | 1029 | CB | ARG | A | 176 | 21.579 | −36.223 | −10.186 | 1.00 | 63.86 | C |
| ATOM | 1030 | CG | ARG | A | 176 | 22.452 | −35.376 | −11.095 | 1.00 | 64.46 | C |
| ATOM | 1031 | CD | ARG | A | 176 | 21.716 | −34.101 | −11.477 | 1.00 | 65.87 | C |
| ATOM | 1032 | NE | ARG | A | 176 | 20.505 | −33.929 | −10.669 | 1.00 | 66.91 | N |
| ATOM | 1033 | CZ | ARG | A | 176 | 19.918 | −32.760 | −10.423 | 1.00 | 67.30 | C |
| ATOM | 1034 | NH1 | ARG | A | 176 | 18.817 | −32.717 | −9.679 | 1.00 | 67.30 | N |
| ATOM | 1035 | NH2 | ARG | A | 176 | 20.435 | −31.635 | −10.912 | 1.00 | 67.62 | N |
| ATOM | 1036 | C | ARG | A | 176 | 23.510 | −37.722 | −9.538 | 1.00 | 63.65 | C |
| ATOM | 1037 | O | ARG | A | 176 | 24.417 | −37.859 | −10.363 | 1.00 | 63.59 | O |
| ATOM | 1038 | N | TRP | A | 177 | 23.733 | −37.633 | −8.229 | 1.00 | 63.54 | N |
| ATOM | 1039 | CA | TRP | A | 177 | 25.080 | −37.773 | −7.668 | 1.00 | 63.37 | C |
| ATOM | 1040 | CB | TRP | A | 177 | 25.025 | −37.527 | −6.160 | 1.00 | 63.31 | C |
| ATOM | 1041 | CG | TRP | A | 177 | 26.347 | −37.583 | −5.454 | 1.00 | 63.26 | C |
| ATOM | 1042 | CD1 | TRP | A | 177 | 27.218 | −36.549 | −5.255 | 1.00 | 62.97 | C |
| ATOM | 1043 | NE1 | TRP | A | 177 | 28.317 | −36.985 | −4.555 | 1.00 | 62.77 | N |
| ATOM | 1044 | CE2 | TRP | A | 177 | 28.169 | −38.320 | −4.280 | 1.00 | 63.09 | C |
| ATOM | 1045 | CD2 | TRP | A | 177 | 26.937 | −38.731 | −4.828 | 1.00 | 63.35 | C |
| ATOM | 1046 | CE3 | TRP | A | 177 | 26.544 | −40.069 | −4.683 | 1.00 | 63.27 | C |
| ATOM | 1047 | CZ3 | TRP | A | 177 | 27.383 | −40.939 | −4.005 | 1.00 | 63.03 | C |
| ATOM | 1048 | CH2 | TRP | A | 177 | 28.604 | −40.500 | −3.473 | 1.00 | 63.04 | C |
| ATOM | 1049 | CZ2 | TRP | A | 177 | 29.014 | −39.199 | −3.599 | 1.00 | 63.18 | C |
| ATOM | 1050 | C | TRP | A | 177 | 25.641 | −39.168 | −7.971 | 1.00 | 63.41 | C |
| ATOM | 1051 | O | TRP | A | 177 | 26.780 | −39.331 | −8.476 | 1.00 | 63.24 | O |
| ATOM | 1052 | N | PHE | A | 178 | 24.826 | −40.177 | −7.672 | 1.00 | 63.59 | N |
| ATOM | 1053 | CA | PHE | A | 178 | 25.161 | −41.545 | −8.047 | 1.00 | 63.57 | C |
| ATOM | 1054 | CB | PHE | A | 178 | 24.094 | −42.526 | −7.558 | 1.00 | 63.59 | C |
| ATOM | 1055 | CG | PHE | A | 178 | 24.285 | −42.971 | −6.137 | 1.00 | 63.89 | C |
| ATOM | 1056 | CD1 | PHE | A | 178 | 25.401 | −43.713 | −5.772 | 1.00 | 64.14 | C |
| ATOM | 1057 | CE1 | PHE | A | 178 | 25.580 | −44.131 | −4.461 | 1.00 | 64.00 | C |
| ATOM | 1058 | CZ | PHE | A | 178 | 24.638 | −43.811 | −3.501 | 1.00 | 63.93 | C |
| ATOM | 1059 | CE2 | PHE | A | 178 | 23.520 | −43.076 | −3.851 | 1.00 | 64.17 | C |
| ATOM | 1060 | CD2 | PHE | A | 178 | 23.346 | −42.660 | −5.166 | 1.00 | 64.18 | C |
| ATOM | 1061 | C | PHE | A | 178 | 25.345 | −41.661 | −9.562 | 1.00 | 63.55 | C |
| ATOM | 1062 | O | PHE | A | 178 | 26.341 | −42.210 | −10.032 | 1.00 | 63.61 | O |
| ATOM | 1063 | N | ALA | A | 179 | 24.391 | −41.133 | −10.325 | 1.00 | 63.51 | N |
| ATOM | 1064 | CA | ALA | A | 179 | 24.479 | −41.170 | −11.783 | 1.00 | 63.38 | C |
| ATOM | 1065 | CB | ALA | A | 179 | 23.344 | −40.377 | −12.410 | 1.00 | 63.38 | C |
| ATOM | 1066 | C | ALA | A | 179 | 25.832 | −40.648 | −12.260 | 1.00 | 63.30 | C |
| ATOM | 1067 | O | ALA | A | 179 | 26.435 | −41.207 | −13.178 | 1.00 | 63.20 | O |
| ATOM | 1068 | N | GLU | A | 180 | 26.303 | −39.574 | −11.631 | 1.00 | 63.33 | N |
| ATOM | 1069 | CA | GLU | A | 180 | 27.635 | −39.051 | −11.916 | 1.00 | 63.25 | C |
| ATOM | 1070 | CB | GLU | A | 180 | 27.923 | −37.789 | −11.102 | 1.00 | 63.23 | C |
| ATOM | 1071 | CG | GLU | A | 180 | 27.232 | −36.535 | −11.594 | 1.00 | 63.57 | C |
| ATOM | 1072 | CD | GLU | A | 180 | 27.433 | −35.365 | −10.650 | 1.00 | 63.57 | C |
| ATOM | 1073 | OE1 | GLU | A | 180 | 28.083 | −35.556 | −9.596 | 1.00 | 63.87 | O |
| ATOM | 1074 | OE2 | GLU | A | 180 | 26.936 | −34.258 | −10.959 | 1.00 | 64.08 | O |
| ATOM | 1075 | C | GLU | A | 180 | 28.695 | −40.090 | −11.595 | 1.00 | 63.07 | C |
| ATOM | 1076 | O | GLU | A | 180 | 29.588 | −40.338 | −12.405 | 1.00 | 63.03 | O |
| ATOM | 1077 | N | ARG | A | 181 | 28.601 | −40.696 | −10.411 | 1.00 | 62.95 | N |
| ATOM | 1078 | CA | ARG | A | 181 | 29.672 | −41.605 | −9.967 | 1.00 | 62.57 | C |
| ATOM | 1079 | CB | ARG | A | 181 | 29.820 | −41.556 | −8.447 | 1.00 | 62.55 | C |
| ATOM | 1080 | CG | ARG | A | 181 | 30.681 | −40.405 | −7.980 | 1.00 | 62.92 | C |
| ATOM | 1081 | CD | ARG | A | 181 | 30.191 | −39.845 | −6.668 | 1.00 | 63.49 | C |
| ATOM | 1082 | NE | ARG | A | 181 | 30.603 | −38.454 | −6.494 | 1.00 | 64.41 | N |
| ATOM | 1083 | CZ | ARG | A | 181 | 29.996 | −37.415 | −7.063 | 1.00 | 64.92 | C |
| ATOM | 1084 | NH1 | ARG | A | 181 | 30.438 | −36.180 | −6.846 | 1.00 | 65.08 | N |
| ATOM | 1085 | NH2 | ARG | A | 181 | 28.943 | −37.606 | −7.849 | 1.00 | 65.13 | N |
| ATOM | 1086 | C | ARG | A | 181 | 29.587 | −43.059 | −10.444 | 1.00 | 62.33 | C |
| ATOM | 1087 | O | ARG | A | 181 | 30.604 | −43.668 | −10.768 | 1.00 | 62.41 | O |
| ATOM | 1088 | N | VAL | A | 182 | 28.377 | −43.601 | −10.499 | 1.00 | 62.04 | N |
| ATOM | 1089 | CA | VAL | A | 182 | 28.153 | −45.040 | −10.663 | 1.00 | 61.56 | C |
| ATOM | 1090 | CB | VAL | A | 182 | 26.718 | −45.378 | −10.249 | 1.00 | 61.45 | C |
| ATOM | 1091 | CG1 | VAL | A | 182 | 26.198 | −46.552 | −11.030 | 1.00 | 61.55 | C |
| ATOM | 1092 | CG2 | VAL | A | 182 | 26.654 | −45.623 | −8.752 | 1.00 | 61.14 | C |
| ATOM | 1093 | C | VAL | A | 182 | 28.430 | −45.596 | −12.065 | 1.00 | 61.44 | C |
| ATOM | 1094 | O | VAL | A | 182 | 28.331 | −44.875 | −13.055 | 1.00 | 61.47 | O |
| ATOM | 1095 | N | ASP | A | 183 | 28.772 | −46.882 | −12.137 | 1.00 | 61.33 | N |
| ATOM | 1096 | CA | ASP | A | 183 | 29.076 | −47.554 | −13.415 | 1.00 | 61.35 | C |
| ATOM | 1097 | CB | ASP | A | 183 | 30.000 | −48.762 | −13.198 | 1.00 | 61.39 | C |
| ATOM | 1098 | CG | ASP | A | 183 | 31.466 | −48.373 | −13.136 | 1.00 | 62.09 | C |
| ATOM | 1099 | OD1 | ASP | A | 183 | 32.248 | −48.863 | −13.981 | 1.00 | 62.54 | O |
| ATOM | 1100 | OD2 | ASP | A | 183 | 31.838 | −47.569 | −12.252 | 1.00 | 63.12 | O |
| ATOM | 1101 | C | ASP | A | 183 | 27.842 | −47.992 | −14.216 | 1.00 | 61.13 | C |
| ATOM | 1102 | O | ASP | A | 183 | 27.771 | −47.778 | −15.428 | 1.00 | 61.05 | O |
| ATOM | 1103 | N | LEU | A | 184 | 26.894 | −48.634 | −13.539 | 1.00 | 60.90 | N |
| ATOM | 1104 | CA | LEU | A | 184 | 25.651 | −49.082 | −14.164 | 1.00 | 60.71 | C |
| ATOM | 1105 | CB | LEU | A | 184 | 25.630 | −50.608 | −14.307 | 1.00 | 60.75 | C |
| ATOM | 1106 | CG | LEU | A | 184 | 26.126 | −51.298 | −15.581 | 1.00 | 60.63 | C |
| ATOM | 1107 | CD1 | LEU | A | 184 | 25.747 | −52.774 | −15.537 | 1.00 | 60.23 | C |
| ATOM | 1108 | CD2 | LEU | A | 184 | 25.555 | −50.648 | −16.828 | 1.00 | 60.26 | C |
| ATOM | 1109 | C | LEU | A | 184 | 24.448 | −48.655 | −13.335 | 1.00 | 60.59 | C |
| ATOM | 1110 | O | LEU | A | 184 | 24.508 | −48.633 | −12.107 | 1.00 | 60.73 | O |
| ATOM | 1111 | N | ILE | A | 185 | 23.351 | −48.320 | −14.005 | 1.00 | 60.33 | N |
| ATOM | 1112 | CA | ILE | A | 185 | 22.119 | −47.996 | −13.310 | 1.00 | 60.09 | C |
| ATOM | 1113 | CB | ILE | A | 185 | 21.670 | −46.560 | −13.589 | 1.00 | 59.94 | C |
| ATOM | 1114 | CG1 | ILE | A | 185 | 22.827 | −45.592 | −13.343 | 1.00 | 59.84 | C |
| ATOM | 1115 | CD1 | ILE | A | 185 | 22.469 | −44.139 | −13.553 | 1.00 | 59.85 | C |
| ATOM | 1116 | CG2 | ILE | A | 185 | 20.483 | −46.203 | −12.718 | 1.00 | 59.66 | C |
| ATOM | 1117 | C | ILE | A | 185 | 21.039 | −48.964 | −13.754 | 1.00 | 60.22 | C |
| ATOM | 1118 | O | ILE | A | 185 | 20.636 | −48.969 | −14.915 | 1.00 | 60.25 | O |
| ATOM | 1119 | N | ILE | A | 186 | 20.586 | −49.799 | −12.830 | 1.00 | 60.39 | N |
| ATOM | 1120 | CA | ILE | A | 186 | 19.557 | −50.777 | −13.145 | 1.00 | 60.66 | C |
| ATOM | 1121 | CB | ILE | A | 186 | 19.785 | −52.100 | −12.400 | 1.00 | 60.60 | C |
| ATOM | 1122 | CG1 | ILE | A | 186 | 21.222 | −52.588 | −12.592 | 1.00 | 60.80 | C |
| ATOM | 1123 | CD1 | ILE | A | 186 | 21.619 | −52.766 | −14.037 | 1.00 | 60.97 | C |
| ATOM | 1124 | CG2 | ILE | A | 186 | 18.808 | −53.141 | −12.881 | 1.00 | 60.56 | C |
| ATOM | 1125 | C | ILE | A | 186 | 18.188 | −50.230 | −12.768 | 1.00 | 60.88 | C |
| ATOM | 1126 | O | ILE | A | 186 | 17.935 | −49.911 | −11.603 | 1.00 | 60.99 | O |
| ATOM | 1127 | N | LEU | A | 187 | 17.312 | −50.107 | −13.757 | 1.00 | 61.05 | N |
| ATOM | 1128 | CA | LEU | A | 187 | 15.945 | −49.672 | −13.507 | 1.00 | 61.25 | C |
| ATOM | 1129 | CB | LEU | A | 187 | 15.555 | −48.542 | −14.458 | 1.00 | 61.17 | C |
| ATOM | 1130 | CG | LEU | A | 187 | 15.836 | −47.107 | −14.010 | 1.00 | 60.76 | C |
| ATOM | 1131 | CD1 | LEU | A | 187 | 17.290 | −46.918 | −13.667 | 1.00 | 60.58 | C |
| ATOM | 1132 | CD2 | LEU | A | 187 | 15.425 | −46.133 | −15.096 | 1.00 | 60.96 | C |
| ATOM | 1133 | C | LEU | A | 187 | 14.980 | −50.850 | −13.649 | 1.00 | 61.68 | C |
| ATOM | 1134 | O | LEU | A | 187 | 14.955 | −51.526 | −14.682 | 1.00 | 61.88 | O |
| ATOM | 1135 | N | LEU | A | 188 | 14.194 | −51.101 | −12.606 | 1.00 | 61.91 | N |
| ATOM | 1136 | CA | LEU | A | 188 | 13.260 | −52.216 | −12.627 | 1.00 | 62.18 | C |
| ATOM | 1137 | CB | LEU | A | 188 | 13.348 | −53.033 | −11.341 | 1.00 | 62.05 | C |
| ATOM | 1138 | CG | LEU | A | 188 | 14.662 | −53.756 | −11.067 | 1.00 | 62.08 | C |
| ATOM | 1139 | CD1 | LEU | A | 188 | 14.375 | −55.091 | −10.405 | 1.00 | 61.97 | C |
| ATOM | 1140 | CD2 | LEU | A | 188 | 15.416 | −53.970 | −12.353 | 1.00 | 62.07 | C |
| ATOM | 1141 | C | LEU | A | 188 | 11.831 | −51.755 | −12.833 | 1.00 | 62.53 | C |
| ATOM | 1142 | O | LEU | A | 188 | 11.372 | −50.801 | −12.205 | 1.00 | 62.71 | O |
| ATOM | 1143 | N | PHE | A | 189 | 11.138 | −52.441 | −13.729 | 1.00 | 62.88 | N |
| ATOM | 1144 | CA | PHE | A | 189 | 9.703 | −52.305 | −13.862 | 1.00 | 63.25 | C |
| ATOM | 1145 | CB | PHE | A | 189 | 9.348 | −51.737 | −15.222 | 1.00 | 63.06 | C |
| ATOM | 1146 | CG | PHE | A | 189 | 9.810 | −50.339 | −15.425 | 1.00 | 63.11 | C |
| ATOM | 1147 | CD1 | PHE | A | 189 | 11.085 | −50.080 | −15.889 | 1.00 | 63.21 | C |
| ATOM | 1148 | CE1 | PHE | A | 189 | 11.513 | −48.781 | −16.081 | 1.00 | 63.18 | C |
| ATOM | 1149 | CZ | PHE | A | 189 | 10.659 | −47.727 | −15.805 | 1.00 | 63.21 | C |
| ATOM | 1150 | CE2 | PHE | A | 189 | 9.385 | −47.974 | −15.339 | 1.00 | 63.09 | C |
| ATOM | 1151 | CD2 | PHE | A | 189 | 8.966 | −49.274 | −15.152 | 1.00 | 63.37 | C |
| ATOM | 1152 | C | PHE | A | 189 | 9.168 | −53.707 | −13.755 | 1.00 | 63.65 | C |
| ATOM | 1153 | O | PHE | A | 189 | 9.798 | −54.631 | −14.257 | 1.00 | 63.87 | O |
| ATOM | 1154 | N | ASP | A | 190 | 8.028 | −53.895 | −13.104 | 1.00 | 64.11 | N |
| ATOM | 1155 | CA | ASP | A | 190 | 7.488 | −55.244 | −13.029 | 1.00 | 64.78 | C |
| ATOM | 1156 | CB | ASP | A | 190 | 7.296 | −55.712 | −11.575 | 1.00 | 64.95 | C |
| ATOM | 1157 | CG | ASP | A | 190 | 5.906 | −55.455 | −11.040 | 1.00 | 65.15 | C |
| ATOM | 1158 | OD1 | ASP | A | 190 | 5.433 | −54.305 | −11.133 | 1.00 | 66.38 | O |
| ATOM | 1159 | OD2 | ASP | A | 190 | 5.299 | −56.403 | −10.497 | 1.00 | 64.63 | O |
| ATOM | 1160 | C | ASP | A | 190 | 6.252 | −55.446 | −13.911 | 1.00 | 65.11 | C |
| ATOM | 1161 | O | ASP | A | 190 | 5.267 | −54.713 | −13.810 | 1.00 | 65.07 | O |
| ATOM | 1162 | N | ALA | A | 191 | 6.348 | −56.441 | −14.793 | 1.00 | 65.57 | N |
| ATOM | 1163 | CA | ALA | A | 191 | 5.325 | −56.747 | −15.793 | 1.00 | 65.98 | C |
| ATOM | 1164 | CB | ALA | A | 191 | 5.777 | −57.918 | −16.644 | 1.00 | 66.05 | C |
| ATOM | 1165 | C | ALA | A | 191 | 4.001 | −57.076 | −15.143 | 1.00 | 66.33 | C |
| ATOM | 1166 | O | ALA | A | 191 | 2.979 | −57.215 | −15.811 | 1.00 | 66.29 | O |
| ATOM | 1167 | N | HIS | A | 192 | 4.039 | −57.206 | −13.826 | 1.00 | 66.88 | N |
| ATOM | 1168 | CA | HIS | A | 192 | 2.885 | −57.594 | −13.058 | 1.00 | 67.53 | C |
| ATOM | 1169 | CB | HIS | A | 192 | 3.359 | −58.253 | −11.773 | 1.00 | 67.72 | C |
| ATOM | 1170 | CG | HIS | A | 192 | 2.252 | −58.756 | −10.912 | 1.00 | 68.70 | C |
| ATOM | 1171 | ND1 | HIS | A | 192 | 1.333 | −59.684 | −11.354 | 1.00 | 69.77 | N |
| ATOM | 1172 | CE1 | HIS | A | 192 | 0.476 | −59.947 | −10.383 | 1.00 | 70.36 | C |
| ATOM | 1173 | NE2 | HIS | A | 192 | 0.812 | −59.227 | −9.327 | 1.00 | 71.03 | N |
| ATOM | 1174 | CD2 | HIS | A | 192 | 1.921 | −58.474 | −9.632 | 1.00 | 69.62 | C |
| ATOM | 1175 | C | HIS | A | 192 | 2.028 | −56.372 | −12.751 | 1.00 | 67.83 | C |
| ATOM | 1176 | O | HIS | A | 192 | 0.806 | −56.409 | −12.890 | 1.00 | 68.11 | O |
| ATOM | 1177 | N | LYS | A | 193 | 2.681 | −55.290 | −12.336 | 1.00 | 68.10 | N |
| ATOM | 1178 | CA | LYS | A | 193 | 2.010 | −54.027 | −12.039 | 1.00 | 68.23 | C |
| ATOM | 1179 | CB | LYS | A | 193 | 2.029 | −53.736 | −10.536 | 1.00 | 68.19 | C |
| ATOM | 1180 | CG | LYS | A | 193 | 0.915 | −54.383 | −9.730 | 1.00 | 68.42 | C |
| ATOM | 1181 | CD | LYS | A | 193 | 0.800 | −53.722 | −8.360 | 1.00 | 68.19 | C |
| ATOM | 1182 | CE | LYS | A | 193 | 0.625 | −52.216 | −8.509 | 1.00 | 68.29 | C |
| ATOM | 1183 | NZ | LYS | A | 193 | 0.868 | −51.484 | −7.239 | 1.00 | 68.54 | N |
| ATOM | 1184 | C | LYS | A | 193 | 2.717 | −52.889 | −12.750 | 1.00 | 68.36 | C |
| ATOM | 1185 | O | LYS | A | 193 | 3.491 | −52.156 | −12.133 | 1.00 | 68.44 | O |
| ATOM | 1186 | N | LEU | A | 194 | 2.459 | −52.730 | −14.040 | 1.00 | 68.56 | N |
| ATOM | 1187 | CA | LEU | A | 194 | 3.100 | −51.653 | −14.781 | 1.00 | 68.79 | C |
| ATOM | 1188 | CB | LEU | A | 194 | 2.892 | −51.826 | −16.282 | 1.00 | 68.92 | C |
| ATOM | 1189 | CG | LEU | A | 194 | 4.052 | −51.373 | −17.167 | 1.00 | 68.90 | C |
| ATOM | 1190 | CD1 | LEU | A | 194 | 3.496 | −50.606 | −18.343 | 1.00 | 69.72 | C |
| ATOM | 1191 | CD2 | LEU | A | 194 | 5.038 | −50.514 | −16.405 | 1.00 | 69.07 | C |
| ATOM | 1192 | C | LEU | A | 194 | 2.570 | −50.294 | −14.335 | 1.00 | 68.86 | C |
| ATOM | 1193 | O | LEU | A | 194 | 1.387 | −49.994 | −14.484 | 1.00 | 68.87 | O |
| ATOM | 1194 | N | GLU | A | 195 | 3.459 | −49.479 | −13.781 | 1.00 | 69.01 | N |
| ATOM | 1195 | CA | GLU | A | 195 | 3.114 | −48.129 | −13.348 | 1.00 | 69.24 | C |
| ATOM | 1196 | CB | GLU | A | 195 | 2.533 | −48.156 | −11.926 | 1.00 | 69.19 | C |
| ATOM | 1197 | CG | GLU | A | 195 | 2.935 | −46.994 | −11.024 | 1.00 | 69.68 | C |
| ATOM | 1198 | CD | GLU | A | 195 | 2.265 | −45.679 | −11.395 | 1.00 | 70.76 | C |
| ATOM | 1199 | OE1 | GLU | A | 195 | 2.217 | −45.345 | −12.597 | 1.00 | 71.25 | O |
| ATOM | 1200 | OE2 | GLU | A | 195 | 1.795 | −44.968 | −10.479 | 1.00 | 70.87 | O |
| ATOM | 1201 | C | GLU | A | 195 | 4.353 | −47.238 | −13.433 | 1.00 | 69.31 | C |
| ATOM | 1202 | O | GLU | A | 195 | 5.439 | −47.641 | −13.016 | 1.00 | 69.43 | O |
| ATOM | 1203 | N | ILE | A | 196 | 4.198 | −46.047 | −14.013 | 1.00 | 69.37 | N |
| ATOM | 1204 | CA | ILE | A | 196 | 5.286 | −45.058 | −14.048 | 1.00 | 69.32 | C |
| ATOM | 1205 | CB | ILE | A | 196 | 5.806 | −44.760 | −15.486 | 1.00 | 69.37 | C |
| ATOM | 1206 | CG1 | ILE | A | 196 | 6.278 | −46.045 | −16.167 | 1.00 | 69.31 | C |
| ATOM | 1207 | CD1 | ILE | A | 196 | 7.156 | −45.796 | −17.375 | 1.00 | 69.52 | C |
| ATOM | 1208 | CG2 | ILE | A | 196 | 6.960 | −43.749 | −15.449 | 1.00 | 69.16 | C |
| ATOM | 1209 | C | ILE | A | 196 | 4.873 | −43.763 | −13.341 | 1.00 | 69.13 | C |
| ATOM | 1210 | O | ILE | A | 196 | 4.292 | −42.853 | −13.938 | 1.00 | 69.00 | O |
| ATOM | 1211 | N | SER | A | 197 | 5.186 | −43.707 | −12.053 | 1.00 | 68.93 | N |
| ATOM | 1212 | CA | SER | A | 197 | 4.839 | −42.579 | −11.215 | 1.00 | 68.80 | C |
| ATOM | 1213 | CB | SER | A | 197 | 5.212 | −42.892 | −9.771 | 1.00 | 68.89 | C |
| ATOM | 1214 | OG | SER | A | 197 | 6.584 | −43.236 | −9.679 | 1.00 | 68.92 | O |
| ATOM | 1215 | C | SER | A | 197 | 5.542 | −41.299 | −11.643 | 1.00 | 68.69 | C |
| ATOM | 1216 | O | SER | A | 197 | 6.556 | −41.331 | −12.345 | 1.00 | 68.50 | O |
| ATOM | 1217 | N | ASP | A | 198 | 4.988 | −40.174 | −11.201 | 1.00 | 68.61 | N |
| ATOM | 1218 | CA | ASP | A | 198 | 5.579 | −38.869 | −11.439 | 1.00 | 68.54 | C |
| ATOM | 1219 | CB | ASP | A | 198 | 4.708 | −37.785 | −10.802 | 1.00 | 68.63 | C |
| ATOM | 1220 | CG | ASP | A | 198 | 5.291 | −36.394 | −10.965 | 1.00 | 69.32 | C |
| ATOM | 1221 | OD1 | ASP | A | 198 | 6.289 | −36.245 | −11.704 | 1.00 | 69.90 | O |
| ATOM | 1222 | OD2 | ASP | A | 198 | 4.747 | −35.445 | −10.354 | 1.00 | 70.07 | O |
| ATOM | 1223 | C | ASP | A | 198 | 6.996 | −38.830 | −10.866 | 1.00 | 68.35 | C |
| ATOM | 1224 | O | ASP | A | 198 | 7.959 | −38.466 | −11.560 | 1.00 | 68.12 | O |
| ATOM | 1225 | N | GLU | A | 199 | 7.116 | −39.220 | −9.600 | 1.00 | 68.16 | N |
| ATOM | 1226 | CA | GLU | A | 199 | 8.406 | −39.264 | −8.926 | 1.00 | 68.12 | C |
| ATOM | 1227 | CB | GLU | A | 199 | 8.256 | −39.928 | −7.558 | 1.00 | 68.13 | C |
| ATOM | 1228 | CG | GLU | A | 199 | 9.527 | −39.956 | −6.717 | 1.00 | 68.49 | C |
| ATOM | 1229 | CD | GLU | A | 199 | 9.283 | −40.500 | −5.315 | 1.00 | 68.60 | C |
| ATOM | 1230 | OE1 | GLU | A | 199 | 8.391 | −39.966 | −4.618 | 1.00 | 69.43 | O |
| ATOM | 1231 | OE2 | GLU | A | 199 | 9.983 | −41.456 | −4.908 | 1.00 | 68.91 | O |
| ATOM | 1232 | C | GLU | A | 199 | 9.450 | −39.994 | −9.774 | 1.00 | 67.92 | C |
| ATOM | 1233 | O | GLU | A | 199 | 10.610 | −39.567 | −9.861 | 1.00 | 67.83 | O |
| ATOM | 1234 | N | PHE | A | 200 | 9.034 | −41.082 | −10.418 | 1.00 | 67.72 | N |
| ATOM | 1235 | CA | PHE | A | 200 | 9.947 | −41.851 | −11.255 | 1.00 | 67.53 | C |
| ATOM | 1236 | CB | PHE | A | 200 | 9.287 | −43.134 | −11.753 | 1.00 | 67.63 | C |
| ATOM | 1237 | CG | PHE | A | 200 | 10.212 | −44.319 | −11.781 | 1.00 | 67.69 | C |
| ATOM | 1238 | CD1 | PHE | A | 200 | 9.890 | −45.482 | −11.102 | 1.00 | 67.87 | C |
| ATOM | 1239 | CE1 | PHE | A | 200 | 10.742 | −46.574 | −11.129 | 1.00 | 68.11 | C |
| ATOM | 1240 | CZ | PHE | A | 200 | 11.934 | −46.505 | −11.828 | 1.00 | 67.82 | C |
| ATOM | 1241 | CE2 | PHE | A | 200 | 12.269 | −45.351 | −12.499 | 1.00 | 67.56 | C |
| ATOM | 1242 | CD2 | PHE | A | 200 | 11.411 | −44.267 | −12.474 | 1.00 | 67.81 | C |
| ATOM | 1243 | C | PHE | A | 200 | 10.455 | −41.020 | −12.432 | 1.00 | 67.43 | C |
| ATOM | 1244 | O | PHE | A | 200 | 11.660 | −40.823 | −12.581 | 1.00 | 67.23 | O |
| ATOM | 1245 | N | SER | A | 201 | 9.541 | −40.522 | −13.262 | 1.00 | 67.37 | N |
| ATOM | 1246 | CA | SER | A | 201 | 9.940 | −39.690 | −14.399 | 1.00 | 67.44 | C |
| ATOM | 1247 | CB | SER | A | 201 | 8.729 | −39.271 | −15.239 | 1.00 | 67.44 | C |
| ATOM | 1248 | OG | SER | A | 201 | 7.921 | −38.337 | −14.551 | 1.00 | 67.77 | O |
| ATOM | 1249 | C | SER | A | 201 | 10.758 | −38.471 | −13.956 | 1.00 | 67.32 | C |
| ATOM | 1250 | O | SER | A | 201 | 11.566 | −37.939 | −14.726 | 1.00 | 67.15 | O |
| ATOM | 1251 | N | GLU | A | 202 | 10.546 | −38.033 | −12.716 | 1.00 | 67.40 | N |
| ATOM | 1252 | CA | GLU | A | 202 | 11.424 | −37.031 | −12.113 | 1.00 | 67.29 | C |
| ATOM | 1253 | CB | GLU | A | 202 | 10.885 | −36.581 | −10.754 | 1.00 | 67.36 | C |
| ATOM | 1254 | CG | GLU | A | 202 | 9.975 | −35.366 | −10.803 | 1.00 | 67.70 | C |
| ATOM | 1255 | CD | GLU | A | 202 | 10.675 | −34.096 | −10.356 | 1.00 | 68.43 | C |
| ATOM | 1256 | OE1 | GLU | A | 202 | 11.599 | −34.193 | −9.520 | 1.00 | 68.71 | O |
| ATOM | 1257 | OE2 | GLU | A | 202 | 10.296 | −33.000 | −10.828 | 1.00 | 68.82 | O |
| ATOM | 1258 | C | GLU | A | 202 | 12.832 | −37.602 | −11.957 | 1.00 | 67.11 | C |
| ATOM | 1259 | O | GLU | A | 202 | 13.824 | −36.963 | −12.332 | 1.00 | 66.97 | O |
| ATOM | 1260 | N | ALA | A | 203 | 12.916 | −38.811 | −11.411 | 1.00 | 67.08 | N |
| ATOM | 1261 | CA | ALA | A | 203 | 14.206 | −39.491 | −11.281 | 1.00 | 66.87 | C |
| ATOM | 1262 | CB | ALA | A | 203 | 14.030 | −40.842 | −10.595 | 1.00 | 66.88 | C |
| ATOM | 1263 | C | ALA | A | 203 | 14.920 | −39.656 | −12.633 | 1.00 | 66.81 | C |
| ATOM | 1264 | O | ALA | A | 203 | 16.110 | −39.327 | −12.771 | 1.00 | 66.36 | O |
| ATOM | 1265 | N | ILE | A | 204 | 14.189 | −40.164 | −13.627 | 1.00 | 66.84 | N |
| ATOM | 1266 | CA | ILE | A | 204 | 14.736 | −40.312 | −14.977 | 1.00 | 66.68 | C |
| ATOM | 1267 | CB | ILE | A | 204 | 13.766 | −41.034 | −15.925 | 1.00 | 66.55 | C |
| ATOM | 1268 | CG1 | ILE | A | 204 | 13.592 | −42.485 | −15.493 | 1.00 | 66.77 | C |
| ATOM | 1269 | CD1 | ILE | A | 204 | 12.277 | −42.763 | −14.821 | 1.00 | 67.14 | C |
| ATOM | 1270 | CG2 | ILE | A | 204 | 14.282 | −40.998 | −17.345 | 1.00 | 66.27 | C |
| ATOM | 1271 | C | ILE | A | 204 | 15.125 | −38.957 | −15.567 | 1.00 | 66.71 | C |
| ATOM | 1272 | O | ILE | A | 204 | 16.102 | −38.850 | −16.312 | 1.00 | 66.75 | O |
| ATOM | 1273 | N | GLY | A | 205 | 14.359 | −37.922 | −15.235 | 1.00 | 66.74 | N |
| ATOM | 1274 | CA | GLY | A | 205 | 14.763 | −36.565 | −15.575 | 1.00 | 66.51 | C |
| ATOM | 1275 | C | GLY | A | 205 | 16.115 | −36.275 | −14.946 | 1.00 | 66.38 | C |
| ATOM | 1276 | O | GLY | A | 205 | 16.957 | −35.597 | −15.541 | 1.00 | 66.39 | O |
| ATOM | 1277 | N | ALA | A | 206 | 16.326 | −36.806 | −13.743 | 1.00 | 66.29 | N |
| ATOM | 1278 | CA | ALA | A | 206 | 17.573 | −36.575 | −13.006 | 1.00 | 65.87 | C |
| ATOM | 1279 | CB | ALA | A | 206 | 17.366 | −36.813 | −11.515 | 1.00 | 65.95 | C |
| ATOM | 1280 | C | ALA | A | 206 | 18.743 | −37.407 | −13.518 | 1.00 | 65.58 | C |
| ATOM | 1281 | O | ALA | A | 206 | 19.900 | −37.072 | −13.280 | 1.00 | 65.48 | O |
| ATOM | 1282 | N | LEU | A | 207 | 18.446 | −38.499 | −14.210 | 1.00 | 65.49 | N |
| ATOM | 1283 | CA | LEU | A | 207 | 19.506 | −39.322 | −14.795 | 1.00 | 65.08 | C |
| ATOM | 1284 | CB | LEU | A | 207 | 19.040 | −40.769 | −14.948 | 1.00 | 65.03 | C |
| ATOM | 1285 | CG | LEU | A | 207 | 19.398 | −41.743 | −13.823 | 1.00 | 65.05 | C |
| ATOM | 1286 | CD1 | LEU | A | 207 | 19.471 | −41.051 | −12.472 | 1.00 | 65.29 | C |
| ATOM | 1287 | CD2 | LEU | A | 207 | 18.398 | −42.885 | −13.782 | 1.00 | 65.61 | C |
| ATOM | 1288 | C | LEU | A | 207 | 20.009 | −38.785 | −16.136 | 1.00 | 64.97 | C |
| ATOM | 1289 | O | LEU | A | 207 | 20.904 | −39.364 | −16.750 | 1.00 | 64.88 | O |
| ATOM | 1290 | N | ARG | A | 208 | 19.433 | −37.674 | −16.582 | 1.00 | 64.92 | N |
| ATOM | 1291 | CA | ARG | A | 208 | 19.771 | −37.083 | −17.879 | 1.00 | 64.83 | C |
| ATOM | 1292 | CB | ARG | A | 208 | 19.025 | −35.751 | −18.064 | 1.00 | 64.82 | C |
| ATOM | 1293 | CG | ARG | A | 208 | 19.634 | −34.791 | −19.087 | 1.00 | 64.69 | C |
| ATOM | 1294 | CD | ARG | A | 208 | 18.525 | −34.039 | −19.840 | 1.00 | 64.59 | C |
| ATOM | 1295 | NE | ARG | A | 208 | 18.746 | −32.581 | −19.922 | 1.00 | 64.85 | N |
| ATOM | 1296 | CZ | ARG | A | 208 | 19.843 | −31.934 | −19.505 | 1.00 | 64.78 | C |
| ATOM | 1297 | NH1 | ARG | A | 208 | 20.873 | −32.594 | −18.958 | 1.00 | 65.02 | N |
| ATOM | 1298 | NH2 | ARG | A | 208 | 19.907 | −30.609 | −19.645 | 1.00 | 64.48 | N |
| ATOM | 1299 | C | ARG | A | 208 | 21.279 | −36.913 | −18.094 | 1.00 | 64.76 | C |
| ATOM | 1300 | O | ARG | A | 208 | 21.993 | −36.423 | −17.218 | 1.00 | 64.84 | O |
| ATOM | 1301 | N | GLY | A | 209 | 21.753 | −37.327 | −19.266 | 1.00 | 64.60 | N |
| ATOM | 1302 | CA | GLY | A | 209 | 23.163 | −37.188 | −19.622 | 1.00 | 64.23 | C |
| ATOM | 1303 | C | GLY | A | 209 | 23.947 | −38.455 | −19.352 | 1.00 | 63.99 | C |
| ATOM | 1304 | O | GLY | A | 209 | 25.131 | −38.553 | −19.677 | 1.00 | 63.84 | O |
| ATOM | 1305 | N | HIS | A | 210 | 23.281 | −39.431 | −18.749 | 1.00 | 63.86 | N |
| ATOM | 1306 | CA | HIS | A | 210 | 23.932 | −40.685 | −18.408 | 1.00 | 63.78 | C |
| ATOM | 1307 | CB | HIS | A | 210 | 24.183 | −40.769 | −16.900 | 1.00 | 63.93 | C |
| ATOM | 1308 | CG | HIS | A | 210 | 25.189 | −39.779 | −16.401 | 1.00 | 64.53 | C |
| ATOM | 1309 | ND1 | HIS | A | 210 | 24.880 | −38.807 | −15.474 | 1.00 | 65.05 | N |
| ATOM | 1310 | CE1 | HIS | A | 210 | 25.955 | −38.079 | −15.228 | 1.00 | 65.36 | C |
| ATOM | 1311 | NE2 | HIS | A | 210 | 26.949 | −38.540 | −15.967 | 1.00 | 65.43 | N |
| ATOM | 1312 | CD2 | HIS | A | 210 | 26.496 | −39.602 | −16.711 | 1.00 | 65.12 | C |
| ATOM | 1313 | C | HIS | A | 210 | 23.132 | −41.891 | −18.878 | 1.00 | 63.46 | C |
| ATOM | 1314 | O | HIS | A | 210 | 23.308 | −42.996 | −18.366 | 1.00 | 63.56 | O |
| ATOM | 1315 | N | GLU | A | 211 | 22.261 | −41.682 | −19.860 | 1.00 | 63.02 | N |
| ATOM | 1316 | CA | GLU | A | 211 | 21.438 | −42.767 | −20.373 | 1.00 | 62.65 | C |
| ATOM | 1317 | CB | GLU | A | 211 | 20.582 | −42.298 | −21.548 | 1.00 | 62.67 | C |
| ATOM | 1318 | CG | GLU | A | 211 | 21.011 | −40.968 | −22.133 | 1.00 | 63.51 | C |
| ATOM | 1319 | CD | GLU | A | 211 | 20.239 | −39.803 | −21.542 | 1.00 | 64.41 | C |
| ATOM | 1320 | OE1 | GLU | A | 211 | 20.737 | −38.656 | −21.593 | 1.00 | 64.72 | O |
| ATOM | 1321 | OE2 | GLU | A | 211 | 19.126 | −40.038 | −21.027 | 1.00 | 64.94 | O |
| ATOM | 1322 | C | GLU | A | 211 | 22.261 | −43.999 | −20.756 | 1.00 | 62.27 | C |
| ATOM | 1323 | O | GLU | A | 211 | 21.780 | −45.128 | −20.648 | 1.00 | 62.45 | O |
| ATOM | 1324 | N | ASP | A | 212 | 23.502 | −43.786 | −21.182 | 1.00 | 61.63 | N |
| ATOM | 1325 | CA | ASP | A | 212 | 24.356 | −44.897 | −21.600 | 1.00 | 61.13 | C |
| ATOM | 1326 | CB | ASP | A | 212 | 25.658 | −44.381 | −22.206 | 1.00 | 61.30 | C |
| ATOM | 1327 | CG | ASP | A | 212 | 26.499 | −43.615 | −21.207 | 1.00 | 62.39 | C |
| ATOM | 1328 | OD1 | ASP | A | 212 | 26.292 | −42.385 | −21.076 | 1.00 | 63.83 | O |
| ATOM | 1329 | OD2 | ASP | A | 212 | 27.374 | −44.239 | −20.561 | 1.00 | 63.08 | O |
| ATOM | 1330 | C | ASP | A | 212 | 24.664 | −45.858 | −20.455 | 1.00 | 60.47 | C |
| ATOM | 1331 | O | ASP | A | 212 | 25.109 | −46.983 | −20.681 | 1.00 | 60.32 | O |
| ATOM | 1332 | N | LYS | A | 213 | 24.427 | −45.410 | −19.229 | 1.00 | 59.79 | N |
| ATOM | 1333 | CA | LYS | A | 213 | 24.701 | −46.226 | −18.058 | 1.00 | 59.18 | C |
| ATOM | 1334 | CB | LYS | A | 213 | 25.212 | −45.361 | −16.909 | 1.00 | 59.14 | C |
| ATOM | 1335 | CG | LYS | A | 213 | 26.566 | −44.724 | −17.136 | 1.00 | 59.05 | C |
| ATOM | 1336 | CD | LYS | A | 213 | 27.080 | −44.131 | −15.831 | 1.00 | 59.11 | C |
| ATOM | 1337 | CE | LYS | A | 213 | 28.308 | −43.262 | −16.033 | 1.00 | 58.78 | C |
| ATOM | 1338 | NZ | LYS | A | 213 | 28.643 | −42.542 | −14.778 | 1.00 | 58.30 | N |
| ATOM | 1339 | C | LYS | A | 213 | 23.456 | −46.962 | −17.601 | 1.00 | 58.86 | C |
| ATOM | 1340 | O | LYS | A | 213 | 23.464 | −47.627 | −16.568 | 1.00 | 58.84 | O |
| ATOM | 1341 | N | ILE | A | 214 | 22.384 | −46.851 | −18.372 | 1.00 | 58.47 | N |
| ATOM | 1342 | CA | ILE | A | 214 | 21.099 | −47.377 | −17.935 | 1.00 | 58.17 | C |
| ATOM | 1343 | CB | ILE | A | 214 | 19.967 | −46.389 | −18.249 | 1.00 | 58.12 | C |
| ATOM | 1344 | CG1 | ILE | A | 214 | 20.170 | −45.098 | −17.459 | 1.00 | 58.10 | C |
| ATOM | 1345 | CD1 | ILE | A | 214 | 19.152 | −44.022 | −17.765 | 1.00 | 58.21 | C |
| ATOM | 1346 | CG2 | ILE | A | 214 | 18.622 | −47.009 | −17.924 | 1.00 | 58.27 | C |
| ATOM | 1347 | C | ILE | A | 214 | 20.745 | −48.735 | −18.531 | 1.00 | 57.94 | C |
| ATOM | 1348 | O | ILE | A | 214 | 20.731 | −48.908 | −19.746 | 1.00 | 58.08 | O |
| ATOM | 1349 | N | ARG | A | 215 | 20.460 | −49.698 | −17.664 | 1.00 | 57.63 | N |
| ATOM | 1350 | CA | ARG | A | 215 | 19.861 | −50.946 | −18.101 | 1.00 | 57.37 | C |
| ATOM | 1351 | CB | ARG | A | 215 | 20.675 | −52.152 | −17.641 | 1.00 | 57.18 | C |
| ATOM | 1352 | CG | ARG | A | 215 | 22.111 | −52.145 | −18.072 | 1.00 | 56.81 | C |
| ATOM | 1353 | CD | ARG | A | 215 | 22.266 | −51.984 | −19.569 | 1.00 | 56.23 | C |
| ATOM | 1354 | NE | ARG | A | 215 | 23.676 | −51.817 | −19.905 | 1.00 | 56.42 | N |
| ATOM | 1355 | CZ | ARG | A | 215 | 24.302 | −50.645 | −19.933 | 1.00 | 56.18 | C |
| ATOM | 1356 | NH1 | ARG | A | 215 | 23.640 | −49.527 | −19.664 | 1.00 | 56.41 | N |
| ATOM | 1357 | NH2 | ARG | A | 215 | 25.590 | −50.591 | −20.234 | 1.00 | 55.83 | N |
| ATOM | 1358 | C | ARG | A | 215 | 18.472 | −51.029 | −17.503 | 1.00 | 57.38 | C |
| ATOM | 1359 | O | ARG | A | 215 | 18.320 | −51.179 | −16.287 | 1.00 | 57.59 | O |
| ATOM | 1360 | N | VAL | A | 216 | 17.461 | −50.913 | −18.357 | 1.00 | 57.17 | N |
| ATOM | 1361 | CA | VAL | A | 216 | 16.075 | −51.040 | −17.934 | 1.00 | 56.79 | C |
| ATOM | 1362 | CB | VAL | A | 216 | 15.154 | −50.313 | −18.912 | 1.00 | 56.68 | C |
| ATOM | 1363 | CG1 | VAL | A | 216 | 13.703 | −50.608 | −18.590 | 1.00 | 56.77 | C |
| ATOM | 1364 | CG2 | VAL | A | 216 | 15.431 | −48.823 | −18.886 | 1.00 | 56.69 | C |
| ATOM | 1365 | C | VAL | A | 216 | 15.688 | −52.506 | −17.941 | 1.00 | 56.67 | C |
| ATOM | 1366 | O | VAL | A | 216 | 15.659 | −53.124 | −18.999 | 1.00 | 56.91 | O |
| ATOM | 1367 | N | VAL | A | 217 | 15.395 | −53.079 | −16.778 | 1.00 | 56.39 | N |
| ATOM | 1368 | CA | VAL | A | 217 | 14.980 | −54.478 | −16.760 | 1.00 | 56.08 | C |
| ATOM | 1369 | CB | VAL | A | 217 | 15.906 | −55.381 | −15.905 | 1.00 | 56.09 | C |
| ATOM | 1370 | CG1 | VAL | A | 217 | 17.104 | −54.600 | −15.412 | 1.00 | 55.83 | C |
| ATOM | 1371 | CG2 | VAL | A | 217 | 15.148 | −56.004 | −14.745 | 1.00 | 56.09 | C |
| ATOM | 1372 | C | VAL | A | 217 | 13.529 | −54.632 | −16.335 | 1.00 | 56.01 | C |
| ATOM | 1373 | O | VAL | A | 217 | 13.112 | −54.152 | −15.275 | 1.00 | 56.14 | O |
| ATOM | 1374 | N | LEU | A | 218 | 12.766 | −55.297 | −17.195 | 1.00 | 55.76 | N |
| ATOM | 1375 | CA | LEU | A | 218 | 11.364 | −55.593 | −16.950 | 1.00 | 55.35 | C |
| ATOM | 1376 | CB | LEU | A | 218 | 10.622 | −55.634 | −18.279 | 1.00 | 55.27 | C |
| ATOM | 1377 | CG | LEU | A | 218 | 9.189 | −56.137 | −18.354 | 1.00 | 55.27 | C |
| ATOM | 1378 | CD1 | LEU | A | 218 | 8.248 | −55.189 | −17.639 | 1.00 | 55.39 | C |
| ATOM | 1379 | CD2 | LEU | A | 218 | 8.817 | −56.270 | −19.817 | 1.00 | 55.68 | C |
| ATOM | 1380 | C | LEU | A | 218 | 11.261 | −56.926 | −16.213 | 1.00 | 55.33 | C |
| ATOM | 1381 | O | LEU | A | 218 | 11.388 | −58.006 | −16.799 | 1.00 | 55.26 | O |
| ATOM | 1382 | N | ASN | A | 219 | 11.040 | −56.830 | −14.911 | 1.00 | 55.33 | N |
| ATOM | 1383 | CA | ASN | A | 219 | 11.089 | −57.970 | −14.018 | 1.00 | 55.16 | C |
| ATOM | 1384 | CB | ASN | A | 219 | 11.392 | −57.479 | −12.607 | 1.00 | 55.29 | C |
| ATOM | 1385 | CG | ASN | A | 219 | 12.091 | −58.516 | −11.774 | 1.00 | 55.94 | C |
| ATOM | 1386 | OD1 | ASN | A | 219 | 12.474 | −59.575 | −12.282 | 1.00 | 56.02 | O |
| ATOM | 1387 | ND2 | ASN | A | 219 | 12.266 | −58.225 | −10.480 | 1.00 | 56.54 | N |
| ATOM | 1388 | C | ASN | A | 219 | 9.807 | −58.789 | −14.008 | 1.00 | 55.04 | C |
| ATOM | 1389 | O | ASN | A | 219 | 8.796 | −58.388 | −14.582 | 1.00 | 54.74 | O |
| ATOM | 1390 | N | LYS | A | 220 | 9.868 | −59.937 | −13.337 | 1.00 | 55.21 | N |
| ATOM | 1391 | CA | LYS | A | 220 | 8.744 | −60.871 | −13.202 | 1.00 | 55.41 | C |
| ATOM | 1392 | CB | LYS | A | 220 | 7.898 | −60.553 | −11.958 | 1.00 | 55.43 | C |
| ATOM | 1393 | CG | LYS | A | 220 | 7.902 | −59.096 | −11.540 | 1.00 | 55.52 | C |
| ATOM | 1394 | CD | LYS | A | 220 | 8.157 | −58.922 | −10.043 | 1.00 | 55.10 | C |
| ATOM | 1395 | CE | LYS | A | 220 | 6.892 | −58.547 | −9.289 | 1.00 | 55.18 | C |
| ATOM | 1396 | NZ | LYS | A | 220 | 7.203 | −57.685 | −8.115 | 1.00 | 54.26 | N |
| ATOM | 1397 | C | LYS | A | 220 | 7.873 | −61.022 | −14.446 | 1.00 | 55.67 | C |
| ATOM | 1398 | O | LYS | A | 220 | 6.652 | −61.107 | −14.352 | 1.00 | 55.78 | O |
| ATOM | 1399 | N | ALA | A | 221 | 8.513 | −61.082 | −15.610 | 1.00 | 56.06 | N |
| ATOM | 1400 | CA | ALA | A | 221 | 7.809 | −61.285 | −16.872 | 1.00 | 56.27 | C |
| ATOM | 1401 | CB | ALA | A | 221 | 8.736 | −61.039 | −18.041 | 1.00 | 56.15 | C |
| ATOM | 1402 | C | ALA | A | 221 | 7.241 | −62.689 | −16.951 | 1.00 | 56.45 | C |
| ATOM | 1403 | O | ALA | A | 221 | 6.458 | −62.996 | −17.841 | 1.00 | 56.60 | O |
| ATOM | 1404 | N | ASP | A | 222 | 7.636 | −63.540 | −16.014 | 1.00 | 56.79 | N |
| ATOM | 1405 | CA | ASP | A | 222 | 7.225 | −64.932 | −16.043 | 1.00 | 57.20 | C |
| ATOM | 1406 | CB | ASP | A | 222 | 8.254 | −65.817 | −15.346 | 1.00 | 57.26 | C |
| ATOM | 1407 | CG | ASP | A | 222 | 8.054 | −65.865 | −13.841 | 1.00 | 58.12 | C |
| ATOM | 1408 | OD1 | ASP | A | 222 | 7.813 | −66.978 | −13.312 | 1.00 | 58.75 | O |
| ATOM | 1409 | OD2 | ASP | A | 222 | 8.117 | −64.791 | −13.191 | 1.00 | 58.98 | O |
| ATOM | 1410 | C | ASP | A | 222 | 5.891 | −65.106 | −15.353 | 1.00 | 57.37 | C |
| ATOM | 1411 | O | ASP | A | 222 | 5.334 | −66.205 | −15.334 | 1.00 | 57.48 | O |
| ATOM | 1412 | N | MET | A | 223 | 5.380 | −64.038 | −14.759 | 1.00 | 57.52 | N |
| ATOM | 1413 | CA | MET | A | 223 | 4.135 | −64.182 | −14.030 | 1.00 | 57.98 | C |
| ATOM | 1414 | CB | MET | A | 223 | 4.286 | −63.751 | −12.571 | 1.00 | 57.98 | C |
| ATOM | 1415 | CG | MET | A | 223 | 4.396 | −62.271 | −12.325 | 1.00 | 58.43 | C |
| ATOM | 1416 | SD | MET | A | 223 | 4.202 | −61.968 | −10.553 | 1.00 | 59.28 | S |
| ATOM | 1417 | CE | MET | A | 223 | 2.734 | −62.938 | −10.188 | 1.00 | 59.20 | C |
| ATOM | 1418 | C | MET | A | 223 | 2.975 | −63.489 | −14.720 | 1.00 | 57.63 | C |
| ATOM | 1419 | O | MET | A | 223 | 2.063 | −62.975 | −14.076 | 1.00 | 57.84 | O |
| ATOM | 1420 | N | VAL | A | 224 | 3.005 | −63.514 | −16.045 | 1.00 | 57.30 | N |
| ATOM | 1421 | CA | VAL | A | 224 | 1.964 | −62.896 | −16.834 | 1.00 | 56.98 | C |
| ATOM | 1422 | CB | VAL | A | 224 | 2.240 | −61.386 | −17.001 | 1.00 | 56.86 | C |
| ATOM | 1423 | CG1 | VAL | A | 224 | 2.971 | −61.108 | −18.298 | 1.00 | 56.82 | C |
| ATOM | 1424 | CG2 | VAL | A | 224 | 0.953 | −60.602 | −16.936 | 1.00 | 56.98 | C |
| ATOM | 1425 | C | VAL | A | 224 | 1.892 | −63.608 | −18.183 | 1.00 | 56.86 | C |
| ATOM | 1426 | O | VAL | A | 224 | 2.917 | −63.957 | −18.764 | 1.00 | 56.87 | O |
| ATOM | 1427 | N | GLU | A | 225 | 0.682 | −63.856 | −18.665 | 1.00 | 56.71 | N |
| ATOM | 1428 | CA | GLU | A | 225 | 0.513 | −64.536 | −19.937 | 1.00 | 56.69 | C |
| ATOM | 1429 | CB | GLU | A | 225 | −0.968 | −64.682 | −20.256 | 1.00 | 57.01 | C |
| ATOM | 1430 | CG | GLU | A | 225 | −1.282 | −65.935 | −21.035 | 1.00 | 58.92 | C |
| ATOM | 1431 | CD | GLU | A | 225 | −1.499 | −67.130 | −20.129 | 1.00 | 61.73 | C |
| ATOM | 1432 | OE1 | GLU | A | 225 | −0.506 | −67.812 | −19.773 | 1.00 | 62.80 | O |
| ATOM | 1433 | OE2 | GLU | A | 225 | −2.671 | −67.378 | −19.761 | 1.00 | 63.64 | O |
| ATOM | 1434 | C | GLU | A | 225 | 1.213 | −63.731 | −21.029 | 1.00 | 56.21 | C |
| ATOM | 1435 | O | GLU | A | 225 | 1.305 | −62.512 | −20.935 | 1.00 | 56.09 | O |
| ATOM | 1436 | N | THR | A | 226 | 1.692 | −64.399 | −22.071 | 1.00 | 55.84 | N |
| ATOM | 1437 | CA | THR | A | 226 | 2.587 | −63.730 | −23.022 | 1.00 | 55.74 | C |
| ATOM | 1438 | CB | THR | A | 226 | 3.293 | −64.715 | −23.989 | 1.00 | 55.64 | C |
| ATOM | 1439 | OG1 | THR | A | 226 | 3.317 | −64.152 | −25.306 | 1.00 | 55.50 | O |
| ATOM | 1440 | CG2 | THR | A | 226 | 2.580 | −66.054 | −24.021 | 1.00 | 56.27 | C |
| ATOM | 1441 | C | THR | A | 226 | 2.027 | −62.487 | −23.756 | 1.00 | 55.65 | C |
| ATOM | 1442 | O | THR | A | 226 | 2.646 | −61.416 | −23.715 | 1.00 | 55.72 | O |
| ATOM | 1443 | N | GLN | A | 227 | 0.882 | −62.618 | −24.426 | 1.00 | 55.48 | N |
| ATOM | 1444 | CA | GLN | A | 227 | 0.245 | −61.451 | −25.055 | 1.00 | 55.18 | C |
| ATOM | 1445 | CB | GLN | A | 227 | −1.218 | −61.737 | −25.375 | 1.00 | 55.14 | C |
| ATOM | 1446 | CG | GLN | A | 227 | −1.496 | −62.165 | −26.797 | 1.00 | 54.85 | C |
| ATOM | 1447 | CD | GLN | A | 227 | −2.945 | −62.543 | −26.991 | 1.00 | 54.43 | C |
| ATOM | 1448 | OE1 | GLN | A | 227 | −3.545 | −63.201 | −26.136 | 1.00 | 53.99 | O |
| ATOM | 1449 | NE2 | GLN | A | 227 | −3.523 | −62.124 | −28.111 | 1.00 | 54.27 | N |
| ATOM | 1450 | C | GLN | A | 227 | 0.309 | −60.245 | −24.126 | 1.00 | 55.20 | C |
| ATOM | 1451 | O | GLN | A | 227 | 0.738 | −59.151 | −24.521 | 1.00 | 55.06 | O |
| ATOM | 1452 | N | GLN | A | 228 | −0.130 | −60.463 | −22.888 | 1.00 | 55.21 | N |
| ATOM | 1453 | CA | GLN | A | 228 | −0.091 | −59.450 | −21.848 | 1.00 | 55.26 | C |
| ATOM | 1454 | CB | GLN | A | 228 | −0.604 | −60.039 | −20.538 | 1.00 | 55.25 | C |
| ATOM | 1455 | CG | GLN | A | 228 | −0.485 | −59.113 | −19.350 | 1.00 | 55.38 | C |
| ATOM | 1456 | CD | GLN | A | 228 | −1.259 | −57.832 | −19.534 | 1.00 | 55.86 | C |
| ATOM | 1457 | OE1 | GLN | A | 228 | −2.266 | −57.794 | −20.243 | 1.00 | 56.42 | O |
| ATOM | 1458 | NE2 | GLN | A | 228 | −0.795 | −56.769 | −18.892 | 1.00 | 56.22 | N |
| ATOM | 1459 | C | GLN | A | 228 | 1.320 | −58.880 | −21.670 | 1.00 | 55.39 | C |
| ATOM | 1460 | O | GLN | A | 228 | 1.504 | −57.660 | −21.581 | 1.00 | 55.39 | O |
| ATOM | 1461 | N | LEU | A | 229 | 2.316 | −59.761 | −21.627 | 1.00 | 55.35 | N |
| ATOM | 1462 | CA | LEU | A | 229 | 3.707 | −59.320 | −21.591 | 1.00 | 55.42 | C |
| ATOM | 1463 | CB | LEU | A | 229 | 4.664 | −60.506 | −21.719 | 1.00 | 55.40 | C |
| ATOM | 1464 | CG | LEU | A | 229 | 5.986 | −60.430 | −20.956 | 1.00 | 55.19 | C |
| ATOM | 1465 | CD1 | LEU | A | 229 | 7.025 | −61.296 | −21.644 | 1.00 | 55.13 | C |
| ATOM | 1466 | CD2 | LEU | A | 229 | 6.476 | −59.001 | −20.843 | 1.00 | 54.53 | C |
| ATOM | 1467 | C | LEU | A | 229 | 3.955 | −58.334 | −22.728 | 1.00 | 55.47 | C |
| ATOM | 1468 | O | LEU | A | 229 | 4.405 | −57.209 | −22.493 | 1.00 | 55.55 | O |
| ATOM | 1469 | N | MET | A | 230 | 3.646 | −58.752 | −23.955 | 1.00 | 55.41 | N |
| ATOM | 1470 | CA | MET | A | 230 | 3.777 | −57.862 | −25.108 | 1.00 | 55.26 | C |
| ATOM | 1471 | CB | MET | A | 230 | 3.182 | −58.497 | −26.364 | 1.00 | 55.44 | C |
| ATOM | 1472 | CG | MET | A | 230 | 3.890 | −59.763 | −26.794 | 1.00 | 55.41 | C |
| ATOM | 1473 | SD | MET | A | 230 | 5.663 | −59.494 | −26.963 | 1.00 | 56.86 | S |
| ATOM | 1474 | CE | MET | A | 230 | 6.272 | −61.082 | −26.388 | 1.00 | 56.24 | C |
| ATOM | 1475 | C | MET | A | 230 | 3.111 | −56.523 | −24.830 | 1.00 | 55.18 | C |
| ATOM | 1476 | O | MET | A | 230 | 3.696 | −55.459 | −25.064 | 1.00 | 55.12 | O |
| ATOM | 1477 | N | ARG | A | 231 | 1.888 | −56.581 | −24.316 | 1.00 | 55.12 | N |
| ATOM | 1478 | CA | ARG | A | 231 | 1.164 | −55.368 | −23.947 | 1.00 | 55.21 | C |
| ATOM | 1479 | CB | ARG | A | 231 | −0.186 | −55.730 | −23.330 | 1.00 | 55.10 | C |
| ATOM | 1480 | CG | ARG | A | 231 | −1.374 | −55.530 | −24.259 | 1.00 | 55.70 | C |
| ATOM | 1481 | CD | ARG | A | 231 | −1.500 | −56.597 | −25.332 | 1.00 | 56.51 | C |
| ATOM | 1482 | NE | ARG | A | 231 | −0.666 | −56.309 | −26.492 | 1.00 | 57.65 | N |
| ATOM | 1483 | CZ | ARG | A | 231 | −0.540 | −57.120 | −27.537 | 1.00 | 57.94 | C |
| ATOM | 1484 | NH1 | ARG | A | 231 | −1.204 | −58.268 | −27.570 | 1.00 | 57.78 | N |
| ATOM | 1485 | NH2 | ARG | A | 231 | 0.252 | −56.782 | −28.546 | 1.00 | 58.31 | N |
| ATOM | 1486 | C | ARG | A | 231 | 1.969 | −54.457 | −23.005 | 1.00 | 55.08 | C |
| ATOM | 1487 | O | ARG | A | 231 | 2.166 | −53.258 | −23.284 | 1.00 | 54.91 | O |
| ATOM | 1488 | N | VAL | A | 232 | 2.443 | −55.032 | −21.901 | 1.00 | 54.99 | N |
| ATOM | 1489 | CA | VAL | A | 232 | 3.204 | −54.267 | −20.917 | 1.00 | 54.96 | C |
| ATOM | 1490 | CB | VAL | A | 232 | 3.520 | −55.100 | −19.664 | 1.00 | 54.88 | C |
| ATOM | 1491 | CG1 | VAL | A | 232 | 4.557 | −54.407 | −18.823 | 1.00 | 55.17 | C |
| ATOM | 1492 | CG2 | VAL | A | 232 | 2.272 | −55.310 | −18.848 | 1.00 | 55.17 | C |
| ATOM | 1493 | C | VAL | A | 232 | 4.494 | −53.702 | −21.518 | 1.00 | 54.91 | C |
| ATOM | 1494 | O | VAL | A | 232 | 4.720 | −52.485 | −21.503 | 1.00 | 54.68 | O |
| ATOM | 1495 | N | TYR | A | 233 | 5.332 | −54.583 | −22.055 | 1.00 | 54.85 | N |
| ATOM | 1496 | CA | TYR | A | 233 | 6.549 | −54.152 | −22.724 | 1.00 | 54.67 | C |
| ATOM | 1497 | CB | TYR | A | 233 | 7.164 | −55.333 | −23.470 | 1.00 | 54.48 | C |
| ATOM | 1498 | CG | TYR | A | 233 | 8.424 | −55.026 | −24.241 | 1.00 | 54.61 | C |
| ATOM | 1499 | CD1 | TYR | A | 233 | 8.799 | −55.811 | −25.321 | 1.00 | 54.70 | C |
| ATOM | 1500 | CE1 | TYR | A | 233 | 9.948 | −55.544 | −26.035 | 1.00 | 54.44 | C |
| ATOM | 1501 | CZ | TYR | A | 233 | 10.736 | −54.480 | −25.678 | 1.00 | 54.26 | C |
| ATOM | 1502 | OH | TYR | A | 233 | 11.877 | −54.219 | −26.394 | 1.00 | 54.47 | O |
| ATOM | 1503 | CE2 | TYR | A | 233 | 10.388 | −53.681 | −24.612 | 1.00 | 54.50 | C |
| ATOM | 1504 | CD2 | TYR | A | 233 | 9.237 | −53.955 | −23.898 | 1.00 | 54.77 | C |
| ATOM | 1505 | C | TYR | A | 233 | 6.256 | −52.964 | −23.657 | 1.00 | 54.73 | C |
| ATOM | 1506 | O | TYR | A | 233 | 6.922 | −51.909 | −23.586 | 1.00 | 54.67 | O |
| ATOM | 1507 | N | GLY | A | 234 | 5.243 | −53.129 | −24.508 | 1.00 | 54.80 | N |
| ATOM | 1508 | CA | GLY | A | 234 | 4.797 | −52.047 | −25.381 | 1.00 | 54.70 | C |
| ATOM | 1509 | C | GLY | A | 234 | 4.565 | −50.754 | −24.615 | 1.00 | 54.80 | C |
| ATOM | 1510 | O | GLY | A | 234 | 5.144 | −49.698 | −24.950 | 1.00 | 54.91 | O |
| ATOM | 1511 | N | ALA | A | 235 | 3.730 | −50.830 | −23.578 | 1.00 | 54.65 | N |
| ATOM | 1512 | CA | ALA | A | 235 | 3.434 | −49.651 | −22.763 | 1.00 | 54.39 | C |
| ATOM | 1513 | CB | ALA | A | 235 | 2.500 | −50.015 | −21.633 | 1.00 | 54.36 | C |
| ATOM | 1514 | C | ALA | A | 235 | 4.708 | −49.002 | −22.216 | 1.00 | 54.45 | C |
| ATOM | 1515 | O | ALA | A | 235 | 4.913 | −47.784 | −22.345 | 1.00 | 54.48 | O |
| ATOM | 1516 | N | LEU | A | 236 | 5.564 | −49.824 | −21.614 | 1.00 | 54.43 | N |
| ATOM | 1517 | CA | LEU | A | 236 | 6.828 | −49.355 | −21.068 | 1.00 | 54.31 | C |
| ATOM | 1518 | CB | LEU | A | 236 | 7.695 | −50.532 | −20.638 | 1.00 | 54.32 | C |
| ATOM | 1519 | CG | LEU | A | 236 | 8.536 | −50.421 | −19.362 | 1.00 | 54.14 | C |
| ATOM | 1520 | CD1 | LEU | A | 236 | 8.970 | −49.007 | −19.059 | 1.00 | 54.03 | C |
| ATOM | 1521 | CD2 | LEU | A | 236 | 9.735 | −51.350 | −19.443 | 1.00 | 53.68 | C |
| ATOM | 1522 | C | LEU | A | 236 | 7.577 | −48.558 | −22.115 | 1.00 | 54.43 | C |
| ATOM | 1523 | O | LEU | A | 236 | 7.874 | −47.383 | −21.910 | 1.00 | 54.51 | O |
| ATOM | 1524 | N | MET | A | 237 | 7.885 | −49.192 | −23.245 | 1.00 | 54.66 | N |
| ATOM | 1525 | CA | MET | A | 237 | 8.702 | −48.506 | −24.253 | 1.00 | 54.81 | C |
| ATOM | 1526 | CB | MET | A | 237 | 9.112 | −49.435 | −25.395 | 1.00 | 54.86 | C |
| ATOM | 1527 | CG | MET | A | 237 | 10.080 | −50.528 | −24.964 | 1.00 | 55.85 | C |
| ATOM | 1528 | SD | MET | A | 237 | 11.647 | −49.943 | −24.265 | 1.00 | 57.46 | S |
| ATOM | 1529 | CE | MET | A | 237 | 12.613 | −49.728 | −25.759 | 1.00 | 56.82 | C |
| ATOM | 1530 | C | MET | A | 237 | 8.030 | −47.250 | −24.786 | 1.00 | 54.74 | C |
| ATOM | 1531 | O | MET | A | 237 | 8.708 | −46.278 | −25.121 | 1.00 | 54.99 | O |
| ATOM | 1532 | N | TRP | A | 238 | 6.704 | −47.261 | −24.855 | 1.00 | 54.61 | N |
| ATOM | 1533 | CA | TRP | A | 238 | 5.983 | −46.061 | −25.264 | 1.00 | 54.49 | C |
| ATOM | 1534 | CB | TRP | A | 238 | 4.496 | −46.358 | −25.263 | 1.00 | 54.42 | C |
| ATOM | 1535 | CG | TRP | A | 238 | 3.815 | −45.961 | −26.503 | 1.00 | 54.35 | C |
| ATOM | 1536 | CD1 | TRP | A | 238 | 3.955 | −46.533 | −27.733 | 1.00 | 54.85 | C |
| ATOM | 1537 | NE1 | TRP | A | 238 | 3.148 | −45.899 | −28.647 | 1.00 | 54.83 | N |
| ATOM | 1538 | CE2 | TRP | A | 238 | 2.463 | −44.903 | −28.006 | 1.00 | 54.14 | C |
| ATOM | 1539 | CD2 | TRP | A | 238 | 2.861 | −44.916 | −26.652 | 1.00 | 53.92 | C |
| ATOM | 1540 | CE3 | TRP | A | 238 | 2.304 | −43.991 | −25.776 | 1.00 | 53.71 | C |
| ATOM | 1541 | CZ3 | TRP | A | 238 | 1.388 | −43.100 | −26.265 | 1.00 | 54.62 | C |
| ATOM | 1542 | CH2 | TRP | A | 238 | 1.014 | −43.109 | −27.617 | 1.00 | 54.60 | C |
| ATOM | 1543 | CZ2 | TRP | A | 238 | 1.539 | −44.004 | −28.499 | 1.00 | 54.05 | C |
| ATOM | 1544 | C | TRP | A | 238 | 6.263 | −44.921 | −24.287 | 1.00 | 54.52 | C |
| ATOM | 1545 | O | TRP | A | 238 | 6.752 | −43.827 | −24.653 | 1.00 | 54.67 | O |
| ATOM | 1546 | N | ALA | A | 239 | 5.951 | −45.206 | −23.028 | 1.00 | 54.59 | N |
| ATOM | 1547 | CA | ALA | A | 239 | 6.148 | −44.262 | −21.941 | 1.00 | 54.74 | C |
| ATOM | 1548 | CB | ALA | A | 239 | 5.781 | −44.915 | −20.617 | 1.00 | 54.68 | C |
| ATOM | 1549 | C | ALA | A | 239 | 7.583 | −43.746 | −21.904 | 1.00 | 54.79 | C |
| ATOM | 1550 | O | ALA | A | 239 | 7.828 | −42.547 | −22.071 | 1.00 | 55.14 | O |
| ATOM | 1551 | N | LEU | A | 240 | 8.525 | −44.661 | −21.680 | 1.00 | 54.72 | N |
| ATOM | 1552 | CA | LEU | A | 240 | 9.939 | −44.311 | −21.643 | 1.00 | 54.66 | C |
| ATOM | 1553 | CB | LEU | A | 240 | 10.807 | −45.565 | −21.563 | 1.00 | 54.59 | C |
| ATOM | 1554 | CG | LEU | A | 240 | 10.956 | −46.206 | −20.184 | 1.00 | 54.30 | C |
| ATOM | 1555 | CD1 | LEU | A | 240 | 11.936 | −47.363 | −20.250 | 1.00 | 54.48 | C |
| ATOM | 1556 | CD2 | LEU | A | 240 | 11.408 | −45.186 | −19.152 | 1.00 | 53.92 | C |
| ATOM | 1557 | C | LEU | A | 240 | 10.317 | −43.482 | −22.860 | 1.00 | 54.72 | C |
| ATOM | 1558 | O | LEU | A | 240 | 10.939 | −42.429 | −22.729 | 1.00 | 54.60 | O |
| ATOM | 1559 | N | GLY | A | 241 | 9.933 | −43.962 | −24.040 | 1.00 | 54.91 | N |
| ATOM | 1560 | CA | GLY | A | 241 | 10.086 | −43.188 | −25.263 | 1.00 | 55.18 | C |
| ATOM | 1561 | C | GLY | A | 241 | 9.742 | −41.726 | −25.038 | 1.00 | 55.50 | C |
| ATOM | 1562 | O | GLY | A | 241 | 10.590 | −40.842 | −25.229 | 1.00 | 55.56 | O |
| ATOM | 1563 | N | LYS | A | 242 | 8.503 | −41.464 | −24.621 | 1.00 | 55.54 | N |
| ATOM | 1564 | CA | LYS | A | 242 | 8.070 | −40.079 | −24.365 | 1.00 | 55.84 | C |
| ATOM | 1565 | CB | LYS | A | 242 | 6.564 | −40.037 | −24.078 | 1.00 | 55.91 | C |
| ATOM | 1566 | CG | LYS | A | 242 | 6.106 | −38.929 | −23.139 | 1.00 | 56.07 | C |
| ATOM | 1567 | CD | LYS | A | 242 | 5.977 | −37.591 | −23.848 | 1.00 | 56.51 | C |
| ATOM | 1568 | CE | LYS | A | 242 | 5.135 | −36.620 | −23.031 | 1.00 | 56.40 | C |
| ATOM | 1569 | NZ | LYS | A | 242 | 3.736 | −37.109 | −22.882 | 1.00 | 56.49 | N |
| ATOM | 1570 | C | LYS | A | 242 | 8.859 | −39.370 | −23.249 | 1.00 | 56.01 | C |
| ATOM | 1571 | O | LYS | A | 242 | 9.107 | −38.168 | −23.328 | 1.00 | 55.92 | O |
| ATOM | 1572 | N | VAL | A | 243 | 9.264 | −40.121 | −22.227 | 1.00 | 56.28 | N |
| ATOM | 1573 | CA | VAL | A | 243 | 9.903 | −39.540 | −21.034 | 1.00 | 56.96 | C |
| ATOM | 1574 | CB | VAL | A | 243 | 9.818 | −40.487 | −19.809 | 1.00 | 57.04 | C |
| ATOM | 1575 | CG1 | VAL | A | 243 | 10.420 | −39.818 | −18.572 | 1.00 | 56.83 | C |
| ATOM | 1576 | CG2 | VAL | A | 243 | 8.375 | −40.904 | −19.550 | 1.00 | 57.52 | C |
| ATOM | 1577 | C | VAL | A | 243 | 11.363 | −39.111 | −21.205 | 1.00 | 57.22 | C |
| ATOM | 1578 | O | VAL | A | 243 | 11.674 | −37.939 | −21.023 | 1.00 | 57.33 | O |
| ATOM | 1579 | N | VAL | A | 244 | 12.256 | −40.051 | −21.528 | 1.00 | 57.66 | N |
| ATOM | 1580 | CA | VAL | A | 244 | 13.693 | −39.748 | −21.602 | 1.00 | 58.09 | C |
| ATOM | 1581 | CB | VAL | A | 244 | 14.578 | −41.016 | −21.585 | 1.00 | 57.99 | C |
| ATOM | 1582 | CG1 | VAL | A | 244 | 13.898 | −42.136 | −20.823 | 1.00 | 58.05 | C |
| ATOM | 1583 | CG2 | VAL | A | 244 | 14.902 | −41.459 | −22.988 | 1.00 | 57.87 | C |
| ATOM | 1584 | C | VAL | A | 244 | 14.057 | −38.882 | −22.812 | 1.00 | 58.63 | C |
| ATOM | 1585 | O | VAL | A | 244 | 15.063 | −38.174 | −22.791 | 1.00 | 58.67 | O |
| ATOM | 1586 | N | GLY | A | 245 | 13.247 | −38.957 | −23.866 | 1.00 | 59.32 | N |
| ATOM | 1587 | CA | GLY | A | 245 | 13.291 | −37.975 | −24.960 | 1.00 | 60.12 | C |
| ATOM | 1588 | C | GLY | A | 245 | 14.427 | −38.053 | −25.967 | 1.00 | 60.64 | C |
| ATOM | 1589 | O | GLY | A | 245 | 14.404 | −37.358 | −26.987 | 1.00 | 60.52 | O |
| ATOM | 1590 | N | THR | A | 246 | 15.422 | −38.891 | −25.681 | 1.00 | 61.23 | N |
| ATOM | 1591 | CA | THR | A | 246 | 16.545 | −39.111 | −26.594 | 1.00 | 61.72 | C |
| ATOM | 1592 | CB | THR | A | 246 | 17.713 | −39.822 | −25.877 | 1.00 | 61.77 | C |
| ATOM | 1593 | OG1 | THR | A | 246 | 18.778 | −40.049 | −26.807 | 1.00 | 62.38 | O |
| ATOM | 1594 | CG2 | THR | A | 246 | 17.264 | −41.160 | −25.307 | 1.00 | 61.63 | C |
| ATOM | 1595 | C | THR | A | 246 | 16.109 | −39.947 | −27.803 | 1.00 | 61.92 | C |
| ATOM | 1596 | O | THR | A | 246 | 15.279 | −40.838 | −27.666 | 1.00 | 61.87 | O |
| ATOM | 1597 | N | PRO | A | 247 | 16.652 | −39.646 | −28.998 | 1.00 | 62.13 | N |
| ATOM | 1598 | CA | PRO | A | 247 | 16.324 | −40.447 | −30.180 | 1.00 | 62.22 | C |
| ATOM | 1599 | CB | PRO | A | 247 | 16.935 | −39.644 | −31.338 | 1.00 | 62.11 | C |
| ATOM | 1600 | CG | PRO | A | 247 | 17.223 | −38.284 | −30.771 | 1.00 | 62.24 | C |
| ATOM | 1601 | CD | PRO | A | 247 | 17.556 | −38.535 | −29.333 | 1.00 | 62.13 | C |
| ATOM | 1602 | C | PRO | A | 247 | 16.922 | −41.857 | −30.135 | 1.00 | 62.38 | C |
| ATOM | 1603 | O | PRO | A | 247 | 16.518 | −42.714 | −30.921 | 1.00 | 62.55 | O |
| ATOM | 1604 | N | GLU | A | 248 | 17.869 | −42.095 | −29.228 | 1.00 | 62.37 | N |
| ATOM | 1605 | CA | GLU | A | 248 | 18.454 | −43.427 | −29.065 | 1.00 | 62.39 | C |
| ATOM | 1606 | CB | GLU | A | 248 | 19.928 | −43.337 | −28.665 | 1.00 | 62.54 | C |
| ATOM | 1607 | CG | GLU | A | 248 | 20.752 | −42.415 | −29.542 | 1.00 | 63.55 | C |
| ATOM | 1608 | CD | GLU | A | 248 | 20.801 | −41.000 | −29.002 | 1.00 | 64.86 | C |
| ATOM | 1609 | OE1 | GLU | A | 248 | 20.533 | −40.048 | −29.772 | 1.00 | 65.06 | O |
| ATOM | 1610 | OE2 | GLU | A | 248 | 21.103 | −40.844 | −27.798 | 1.00 | 65.69 | O |
| ATOM | 1611 | C | GLU | A | 248 | 17.687 | −44.235 | −28.030 | 1.00 | 62.14 | C |
| ATOM | 1612 | O | GLU | A | 248 | 17.524 | −43.801 | −26.892 | 1.00 | 62.25 | O |
| ATOM | 1613 | N | VAL | A | 249 | 17.234 | −45.419 | −28.429 | 1.00 | 61.87 | N |
| ATOM | 1614 | CA | VAL | A | 249 | 16.374 | −46.254 | −27.590 | 1.00 | 61.58 | C |
| ATOM | 1615 | CB | VAL | A | 249 | 15.486 | −47.184 | −28.459 | 1.00 | 61.51 | C |
| ATOM | 1616 | CG1 | VAL | A | 249 | 16.221 | −47.608 | −29.720 | 1.00 | 61.83 | C |
| ATOM | 1617 | CG2 | VAL | A | 249 | 15.035 | −48.396 | −27.674 | 1.00 | 61.54 | C |
| ATOM | 1618 | C | VAL | A | 249 | 17.134 | −47.072 | −26.538 | 1.00 | 61.44 | C |
| ATOM | 1619 | O | VAL | A | 249 | 18.170 | −47.671 | −26.823 | 1.00 | 61.45 | O |
| ATOM | 1620 | N | LEU | A | 250 | 16.608 | −47.082 | −25.318 | 1.00 | 61.36 | N |
| ATOM | 1621 | CA | LEU | A | 250 | 17.189 | −47.851 | −24.224 | 1.00 | 61.27 | C |
| ATOM | 1622 | CB | LEU | A | 250 | 16.662 | −47.347 | −22.881 | 1.00 | 61.02 | C |
| ATOM | 1623 | CG | LEU | A | 250 | 17.582 | −46.481 | −22.021 | 1.00 | 60.92 | C |
| ATOM | 1624 | CD1 | LEU | A | 250 | 18.366 | −45.482 | −22.848 | 1.00 | 61.76 | C |
| ATOM | 1625 | CD2 | LEU | A | 250 | 16.777 | −45.765 | −20.962 | 1.00 | 61.02 | C |
| ATOM | 1626 | C | LEU | A | 250 | 16.888 | −49.335 | −24.361 | 1.00 | 61.49 | C |
| ATOM | 1627 | O | LEU | A | 250 | 15.789 | −49.725 | −24.754 | 1.00 | 61.64 | O |
| ATOM | 1628 | N | ARG | A | 251 | 17.871 | −50.161 | −24.025 | 1.00 | 61.69 | N |
| ATOM | 1629 | CA | ARG | A | 251 | 17.693 | −51.606 | −24.018 | 1.00 | 61.74 | C |
| ATOM | 1630 | CB | ARG | A | 251 | 19.060 | −52.294 | −24.056 | 1.00 | 61.77 | C |
| ATOM | 1631 | CG | ARG | A | 251 | 19.056 | −53.780 | −23.705 | 1.00 | 61.82 | C |
| ATOM | 1632 | CD | ARG | A | 251 | 18.614 | −54.629 | −24.885 | 1.00 | 61.89 | C |
| ATOM | 1633 | NE | ARG | A | 251 | 19.429 | −55.833 | −25.010 | 1.00 | 61.71 | N |
| ATOM | 1634 | CZ | ARG | A | 251 | 19.057 | −57.040 | −24.598 | 1.00 | 61.63 | C |
| ATOM | 1635 | NH1 | ARG | A | 251 | 17.874 | −57.216 | −24.030 | 1.00 | 61.74 | N |
| ATOM | 1636 | NH2 | ARG | A | 251 | 19.875 | −58.072 | −24.751 | 1.00 | 61.58 | N |
| ATOM | 1637 | C | ARG | A | 251 | 16.946 | −52.033 | −22.762 | 1.00 | 61.79 | C |
| ATOM | 1638 | O | ARG | A | 251 | 17.320 | −51.641 | −21.656 | 1.00 | 61.72 | O |
| ATOM | 1639 | N | VAL | A | 252 | 15.890 | −52.825 | −22.929 | 1.00 | 61.91 | N |
| ATOM | 1640 | CA | VAL | A | 252 | 15.265 | −53.487 | −21.778 | 1.00 | 62.11 | C |
| ATOM | 1641 | CB | VAL | A | 252 | 13.724 | −53.317 | −21.741 | 1.00 | 62.15 | C |
| ATOM | 1642 | CG1 | VAL | A | 252 | 13.357 | −51.846 | −21.684 | 1.00 | 62.31 | C |
| ATOM | 1643 | CG2 | VAL | A | 252 | 13.085 | −53.976 | −22.949 | 1.00 | 62.44 | C |
| ATOM | 1644 | C | VAL | A | 252 | 15.625 | −54.972 | −21.739 | 1.00 | 61.92 | C |
| ATOM | 1645 | O | VAL | A | 252 | 15.675 | −55.633 | −22.776 | 1.00 | 61.98 | O |
| ATOM | 1646 | N | TYR | A | 253 | 15.894 | −55.482 | −20.541 | 1.00 | 61.76 | N |
| ATOM | 1647 | CA | TYR | A | 253 | 16.191 | −56.898 | −20.351 | 1.00 | 61.60 | C |
| ATOM | 1648 | CB | TYR | A | 253 | 17.414 | −57.072 | −19.449 | 1.00 | 61.60 | C |
| ATOM | 1649 | CG | TYR | A | 253 | 18.679 | −56.478 | −20.036 | 1.00 | 62.19 | C |
| ATOM | 1650 | CD1 | TYR | A | 253 | 19.206 | −55.293 | −19.549 | 1.00 | 62.55 | C |
| ATOM | 1651 | CE1 | TYR | A | 253 | 20.359 | −54.749 | −20.091 | 1.00 | 62.44 | C |
| ATOM | 1652 | CZ | TYR | A | 253 | 20.993 | −55.384 | −21.132 | 1.00 | 61.97 | C |
| ATOM | 1653 | OH | TYR | A | 253 | 22.139 | −54.844 | −21.673 | 1.00 | 62.06 | O |
| ATOM | 1654 | CE2 | TYR | A | 253 | 20.490 | −56.557 | −21.632 | 1.00 | 62.23 | C |
| ATOM | 1655 | CD2 | TYR | A | 253 | 19.341 | −57.098 | −21.088 | 1.00 | 62.57 | C |
| ATOM | 1656 | C | TYR | A | 253 | 14.976 | −57.588 | −19.749 | 1.00 | 61.37 | C |
| ATOM | 1657 | O | TYR | A | 253 | 14.563 | −57.252 | −18.640 | 1.00 | 61.52 | O |
| ATOM | 1658 | N | ILE | A | 254 | 14.400 | −58.539 | −20.484 | 1.00 | 60.85 | N |
| ATOM | 1659 | CA | ILE | A | 254 | 13.152 | −59.177 | −20.068 | 1.00 | 60.38 | C |
| ATOM | 1660 | CB | ILE | A | 254 | 12.245 | −59.455 | −21.275 | 1.00 | 60.34 | C |
| ATOM | 1661 | CG1 | ILE | A | 254 | 12.301 | −58.278 | −22.251 | 1.00 | 60.50 | C |
| ATOM | 1662 | CD1 | ILE | A | 254 | 11.238 | −58.319 | −23.329 | 1.00 | 60.93 | C |
| ATOM | 1663 | CG2 | ILE | A | 254 | 10.821 | −59.735 | −20.821 | 1.00 | 59.77 | C |
| ATOM | 1664 | C | ILE | A | 254 | 13.385 | −60.486 | −19.324 | 1.00 | 60.21 | C |
| ATOM | 1665 | O | ILE | A | 254 | 13.958 | −61.423 | −19.867 | 1.00 | 60.30 | O |
| ATOM | 1666 | N | GLY | A | 255 | 12.931 | −60.558 | −18.079 | 1.00 | 59.92 | N |
| ATOM | 1667 | CA | GLY | A | 255 | 13.090 | −61.789 | −17.310 | 1.00 | 59.62 | C |
| ATOM | 1668 | C | GLY | A | 255 | 12.484 | −61.725 | −15.923 | 1.00 | 59.23 | C |
| ATOM | 1669 | O | GLY | A | 255 | 11.792 | −60.766 | −15.585 | 1.00 | 59.30 | O |
| ATOM | 1670 | N | SER | A | 256 | 12.735 | −62.759 | −15.126 | 1.00 | 58.78 | N |
| ATOM | 1671 | CA | SER | A | 256 | 12.342 | −62.757 | −13.723 | 1.00 | 58.69 | C |
| ATOM | 1672 | CB | SER | A | 256 | 11.081 | −63.593 | −13.486 | 1.00 | 58.72 | C |
| ATOM | 1673 | OG | SER | A | 256 | 11.098 | −64.776 | −14.260 | 1.00 | 59.59 | O |
| ATOM | 1674 | C | SER | A | 256 | 13.500 | −63.204 | −12.829 | 1.00 | 58.34 | C |
| ATOM | 1675 | O | SER | A | 256 | 13.731 | −64.392 | −12.606 | 1.00 | 58.22 | O |
| ATOM | 1676 | N | PHE | A | 257 | 14.207 | −62.212 | −12.305 | 1.00 | 58.01 | N |
| ATOM | 1677 | CA | PHE | A | 257 | 15.459 | −62.410 | −11.606 | 1.00 | 57.54 | C |
| ATOM | 1678 | CB | PHE | A | 257 | 16.256 | −61.107 | −11.660 | 1.00 | 57.11 | C |
| ATOM | 1679 | CG | PHE | A | 257 | 16.501 | −60.627 | −13.062 | 1.00 | 56.81 | C |
| ATOM | 1680 | CD1 | PHE | A | 257 | 17.747 | −60.763 | −13.651 | 1.00 | 56.40 | C |
| ATOM | 1681 | CE1 | PHE | A | 257 | 17.969 | −60.338 | −14.950 | 1.00 | 55.40 | C |
| ATOM | 1682 | CZ | PHE | A | 257 | 16.941 | −59.781 | −15.679 | 1.00 | 55.70 | C |
| ATOM | 1683 | CE2 | PHE | A | 257 | 15.693 | −59.650 | −15.119 | 1.00 | 55.89 | C |
| ATOM | 1684 | CD2 | PHE | A | 257 | 15.473 | −60.077 | −13.813 | 1.00 | 56.91 | C |
| ATOM | 1685 | C | PHE | A | 257 | 15.300 | −62.948 | −10.177 | 1.00 | 57.57 | C |
| ATOM | 1686 | O | PHE | A | 257 | 15.433 | −62.216 | −9.193 | 1.00 | 57.54 | O |
| ATOM | 1687 | N | TRP | A | 258 | 15.008 | −64.243 | −10.090 | 1.00 | 57.58 | N |
| ATOM | 1688 | CA | TRP | A | 258 | 14.989 | −64.966 | −8.821 | 1.00 | 57.65 | C |
| ATOM | 1689 | CB | TRP | A | 258 | 13.858 | −64.464 | −7.917 | 1.00 | 57.76 | C |
| ATOM | 1690 | CG | TRP | A | 258 | 12.478 | −64.843 | −8.339 | 1.00 | 57.87 | C |
| ATOM | 1691 | CD1 | TRP | A | 258 | 12.077 | −65.277 | −9.572 | 1.00 | 58.43 | C |
| ATOM | 1692 | NE1 | TRP | A | 258 | 10.723 | −65.520 | −9.572 | 1.00 | 58.60 | N |
| ATOM | 1693 | CE2 | TRP | A | 258 | 10.222 | −65.229 | −8.329 | 1.00 | 58.63 | C |
| ATOM | 1694 | CD2 | TRP | A | 258 | 11.299 | −64.791 | −7.530 | 1.00 | 58.19 | C |
| ATOM | 1695 | CE3 | TRP | A | 258 | 11.050 | −64.430 | −6.201 | 1.00 | 58.21 | C |
| ATOM | 1696 | CZ3 | TRP | A | 258 | 9.753 | −64.513 | −5.720 | 1.00 | 58.20 | C |
| ATOM | 1697 | CH2 | TRP | A | 258 | 8.703 | −64.949 | −6.539 | 1.00 | 58.12 | C |
| ATOM | 1698 | CZ2 | TRP | A | 258 | 8.916 | −65.313 | −7.842 | 1.00 | 58.37 | C |
| ATOM | 1699 | C | TRP | A | 258 | 14.928 | −66.479 | −9.058 | 1.00 | 57.67 | C |
| ATOM | 1700 | O | TRP | A | 258 | 14.436 | −66.937 | −10.093 | 1.00 | 57.71 | O |
| ATOM | 1701 | N | SER | A | 259 | 15.443 | −67.254 | −8.107 | 1.00 | 57.66 | N |
| ATOM | 1702 | CA | SER | A | 259 | 15.578 | −68.706 | −8.285 | 1.00 | 57.55 | C |
| ATOM | 1703 | CB | SER | A | 259 | 16.660 | −69.255 | −7.356 | 1.00 | 57.60 | C |
| ATOM | 1704 | OG | SER | A | 259 | 17.916 | −68.667 | −7.651 | 1.00 | 58.07 | O |
| ATOM | 1705 | C | SER | A | 259 | 14.274 | −69.478 | −8.081 | 1.00 | 57.36 | C |
| ATOM | 1706 | O | SER | A | 259 | 14.283 | −70.686 | −7.836 | 1.00 | 57.37 | O |
| ATOM | 1707 | N | GLN | A | 260 | 13.156 | −68.775 | −8.194 | 1.00 | 57.11 | N |
| ATOM | 1708 | CA | GLN | A | 260 | 11.850 | −69.384 | −8.021 | 1.00 | 56.90 | C |
| ATOM | 1709 | CB | GLN | A | 260 | 10.841 | −68.302 | −7.643 | 1.00 | 57.05 | C |
| ATOM | 1710 | CG | GLN | A | 260 | 9.862 | −68.687 | −6.541 | 1.00 | 58.16 | C |
| ATOM | 1711 | CD | GLN | A | 260 | 10.543 | −68.952 | −5.204 | 1.00 | 59.01 | C |
| ATOM | 1712 | OE1 | GLN | A | 260 | 11.611 | −68.404 | −4.913 | 1.00 | 59.19 | O |
| ATOM | 1713 | NE2 | GLN | A | 260 | 9.922 | −69.798 | −4.384 | 1.00 | 58.96 | N |
| ATOM | 1714 | C | GLN | A | 260 | 11.441 | −70.055 | −9.327 | 1.00 | 56.43 | C |
| ATOM | 1715 | O | GLN | A | 260 | 11.879 | −69.640 | −10.396 | 1.00 | 56.50 | O |
| ATOM | 1716 | N | PRO | A | 261 | 10.623 | −71.113 | −9.249 | 1.00 | 56.02 | N |
| ATOM | 1717 | CA | PRO | A | 261 | 10.059 | −71.724 | −10.452 | 1.00 | 55.81 | C |
| ATOM | 1718 | CB | PRO | A | 261 | 9.110 | −72.785 | −9.893 | 1.00 | 55.75 | C |
| ATOM | 1719 | CG | PRO | A | 261 | 9.690 | −73.132 | −8.570 | 1.00 | 55.87 | C |
| ATOM | 1720 | CD | PRO | A | 261 | 10.223 | −71.832 | −8.030 | 1.00 | 56.03 | C |
| ATOM | 1721 | C | PRO | A | 261 | 9.292 | −70.729 | −11.319 | 1.00 | 55.59 | C |
| ATOM | 1722 | O | PRO | A | 261 | 8.921 | −69.652 | −10.859 | 1.00 | 55.74 | O |
| ATOM | 1723 | N | LEU | A | 262 | 9.057 | −71.092 | −12.570 | 1.00 | 55.30 | N |
| ATOM | 1724 | CA | LEU | A | 262 | 8.463 | −70.173 | −13.518 | 1.00 | 55.12 | C |
| ATOM | 1725 | CB | LEU | A | 262 | 9.026 | −70.457 | −14.905 | 1.00 | 55.09 | C |
| ATOM | 1726 | CG | LEU | A | 262 | 9.764 | −69.356 | −15.667 | 1.00 | 54.85 | C |
| ATOM | 1727 | CD1 | LEU | A | 262 | 10.497 | −68.395 | −14.741 | 1.00 | 54.87 | C |
| ATOM | 1728 | CD2 | LEU | A | 262 | 10.742 | −70.010 | −16.607 | 1.00 | 54.29 | C |
| ATOM | 1729 | C | LEU | A | 262 | 6.966 | −70.386 | −13.513 | 1.00 | 55.14 | C |
| ATOM | 1730 | O | LEU | A | 262 | 6.508 | −71.518 | −13.615 | 1.00 | 55.24 | O |
| ATOM | 1731 | N | LEU | A | 263 | 6.198 | −69.309 | −13.379 | 1.00 | 55.24 | N |
| ATOM | 1732 | CA | LEU | A | 263 | 4.743 | −69.433 | −13.356 | 1.00 | 55.20 | C |
| ATOM | 1733 | CB | LEU | A | 263 | 4.076 | −68.110 | −12.967 | 1.00 | 55.14 | C |
| ATOM | 1734 | CG | LEU | A | 263 | 2.706 | −68.176 | −12.271 | 1.00 | 55.03 | C |
| ATOM | 1735 | CD1 | LEU | A | 263 | 2.132 | −66.781 | −12.099 | 1.00 | 54.72 | C |
| ATOM | 1736 | CD2 | LEU | A | 263 | 1.706 | −69.056 | −13.010 | 1.00 | 55.07 | C |
| ATOM | 1737 | C | LEU | A | 263 | 4.266 | −69.875 | −14.729 | 1.00 | 55.30 | C |
| ATOM | 1738 | O | LEU | A | 263 | 3.879 | −71.025 | −14.921 | 1.00 | 55.01 | O |
| ATOM | 1739 | N | VAL | A | 264 | 4.298 | −68.949 | −15.680 | 1.00 | 55.62 | N |
| ATOM | 1740 | CA | VAL | A | 264 | 3.950 | −69.260 | −17.059 | 1.00 | 55.97 | C |
| ATOM | 1741 | CB | VAL | A | 264 | 3.016 | −68.194 | −17.669 | 1.00 | 55.83 | C |
| ATOM | 1742 | CG1 | VAL | A | 264 | 3.489 | −66.801 | −17.317 | 1.00 | 55.89 | C |
| ATOM | 1743 | CG2 | VAL | A | 264 | 2.898 | −68.376 | −19.175 | 1.00 | 56.05 | C |
| ATOM | 1744 | C | VAL | A | 264 | 5.228 | −69.448 | −17.876 | 1.00 | 56.23 | C |
| ATOM | 1745 | O | VAL | A | 264 | 5.882 | −68.484 | −18.251 | 1.00 | 56.29 | O |
| ATOM | 1746 | N | PRO | A | 265 | 5.574 | −70.711 | −18.161 | 1.00 | 56.68 | N |
| ATOM | 1747 | CA | PRO | A | 265 | 6.910 | −71.109 | −18.587 | 1.00 | 57.10 | C |
| ATOM | 1748 | CB | PRO | A | 265 | 6.917 | −72.607 | −18.282 | 1.00 | 57.09 | C |
| ATOM | 1749 | CG | PRO | A | 265 | 5.512 | −73.022 | −18.537 | 1.00 | 56.77 | C |
| ATOM | 1750 | CD | PRO | A | 265 | 4.657 | −71.863 | −18.091 | 1.00 | 56.60 | C |
| ATOM | 1751 | C | PRO | A | 265 | 7.228 | −70.900 | −20.067 | 1.00 | 57.60 | C |
| ATOM | 1752 | O | PRO | A | 265 | 8.402 | −70.934 | −20.449 | 1.00 | 57.55 | O |
| ATOM | 1753 | N | ASP | A | 266 | 6.210 | −70.686 | −20.896 | 1.00 | 58.11 | N |
| ATOM | 1754 | CA | ASP | A | 266 | 6.421 | −70.770 | −22.341 | 1.00 | 58.60 | C |
| ATOM | 1755 | CB | ASP | A | 266 | 5.111 | −71.053 | −23.108 | 1.00 | 58.96 | C |
| ATOM | 1756 | CG | ASP | A | 266 | 3.967 | −70.141 | −22.702 | 1.00 | 59.26 | C |
| ATOM | 1757 | OD1 | ASP | A | 266 | 4.064 | −68.920 | −22.943 | 1.00 | 60.12 | O |
| ATOM | 1758 | OD2 | ASP | A | 266 | 2.955 | −70.659 | −22.177 | 1.00 | 59.63 | O |
| ATOM | 1759 | C | ASP | A | 266 | 7.205 | −69.617 | −22.951 | 1.00 | 58.63 | C |
| ATOM | 1760 | O | ASP | A | 266 | 6.978 | −69.238 | −24.092 | 1.00 | 58.80 | O |
| ATOM | 1761 | N | ASN | A | 267 | 8.151 | −69.085 | −22.190 | 1.00 | 58.79 | N |
| ATOM | 1762 | CA | ASN | A | 267 | 8.998 | −68.002 | −22.663 | 1.00 | 58.87 | C |
| ATOM | 1763 | CB | ASN | A | 267 | 8.339 | −66.657 | −22.369 | 1.00 | 58.66 | C |
| ATOM | 1764 | CG | ASN | A | 267 | 7.617 | −66.102 | −23.561 | 1.00 | 58.19 | C |
| ATOM | 1765 | OD1 | ASN | A | 267 | 8.090 | −66.229 | −24.686 | 1.00 | 58.89 | O |
| ATOM | 1766 | ND2 | ASN | A | 267 | 6.467 | −65.483 | −23.331 | 1.00 | 57.38 | N |
| ATOM | 1767 | C | ASN | A | 267 | 10.387 | −68.040 | −22.037 | 1.00 | 59.27 | C |
| ATOM | 1768 | O | ASN | A | 267 | 11.117 | −67.045 | −22.065 | 1.00 | 59.19 | O |
| ATOM | 1769 | N | ARG | A | 268 | 10.763 | −69.187 | −21.477 | 1.00 | 59.60 | N |
| ATOM | 1770 | CA | ARG | A | 268 | 11.957 | −69.204 | −20.649 | 1.00 | 59.99 | C |
| ATOM | 1771 | CB | ARG | A | 268 | 11.986 | −70.400 | −19.683 | 1.00 | 60.07 | C |
| ATOM | 1772 | CG | ARG | A | 268 | 12.662 | −71.672 | −20.115 | 1.00 | 60.90 | C |
| ATOM | 1773 | CD | ARG | A | 268 | 13.141 | −72.379 | −18.844 | 1.00 | 63.18 | C |
| ATOM | 1774 | NE | ARG | A | 268 | 13.096 | −73.840 | −18.898 | 1.00 | 65.28 | N |
| ATOM | 1775 | CZ | ARG | A | 268 | 11.996 | −74.570 | −18.704 | 1.00 | 66.78 | C |
| ATOM | 1776 | NH1 | ARG | A | 268 | 10.830 | −73.975 | −18.460 | 1.00 | 66.33 | N |
| ATOM | 1777 | NH2 | ARG | A | 268 | 12.057 | −75.902 | −18.764 | 1.00 | 67.27 | N |
| ATOM | 1778 | C | ARG | A | 268 | 13.241 | −69.016 | −21.440 | 1.00 | 60.01 | C |
| ATOM | 1779 | O | ARG | A | 268 | 14.156 | −68.351 | −20.970 | 1.00 | 60.20 | O |
| ATOM | 1780 | N | ARG | A | 269 | 13.287 | −69.550 | −22.654 | 1.00 | 60.18 | N |
| ATOM | 1781 | CA | ARG | A | 269 | 14.414 | −69.298 | −23.548 | 1.00 | 60.31 | C |
| ATOM | 1782 | CB | ARG | A | 269 | 14.079 | −69.759 | −24.967 | 1.00 | 60.47 | C |
| ATOM | 1783 | CG | ARG | A | 269 | 14.873 | −70.966 | −25.444 | 1.00 | 61.18 | C |
| ATOM | 1784 | CD | ARG | A | 269 | 16.252 | −70.541 | −25.953 | 1.00 | 62.47 | C |
| ATOM | 1785 | NE | ARG | A | 269 | 17.234 | −70.391 | −24.878 | 1.00 | 62.90 | N |
| ATOM | 1786 | CZ | ARG | A | 269 | 18.457 | −70.919 | −24.900 | 1.00 | 62.99 | C |
| ATOM | 1787 | NH1 | ARG | A | 269 | 18.866 | −71.619 | −25.952 | 1.00 | 62.79 | N |
| ATOM | 1788 | NH2 | ARG | A | 269 | 19.281 | −70.729 | −23.875 | 1.00 | 62.86 | N |
| ATOM | 1789 | C | ARG | A | 269 | 14.784 | −67.818 | −23.554 | 1.00 | 60.15 | C |
| ATOM | 1790 | O | ARG | A | 269 | 15.943 | −67.449 | −23.343 | 1.00 | 60.18 | O |
| ATOM | 1791 | N | LEU | A | 270 | 13.781 | −66.978 | −23.789 | 1.00 | 60.00 | N |
| ATOM | 1792 | CA | LEU | A | 270 | 13.970 | −65.539 | −23.848 | 1.00 | 59.87 | C |
| ATOM | 1793 | CB | LEU | A | 270 | 12.637 | −64.857 | −24.150 | 1.00 | 59.80 | C |
| ATOM | 1794 | CG | LEU | A | 270 | 12.602 | −63.460 | −24.777 | 1.00 | 59.73 | C |
| ATOM | 1795 | CD1 | LEU | A | 270 | 12.448 | −62.370 | −23.734 | 1.00 | 59.80 | C |
| ATOM | 1796 | CD2 | LEU | A | 270 | 13.817 | −63.214 | −25.654 | 1.00 | 59.78 | C |
| ATOM | 1797 | C | LEU | A | 270 | 14.543 | −65.031 | −22.537 | 1.00 | 59.92 | C |
| ATOM | 1798 | O | LEU | A | 270 | 15.516 | −64.287 | −22.525 | 1.00 | 60.09 | O |
| ATOM | 1799 | N | PHE | A | 271 | 13.948 | −65.455 | −21.429 | 1.00 | 60.22 | N |
| ATOM | 1800 | CA | PHE | A | 271 | 14.377 | −65.006 | −20.109 | 1.00 | 60.39 | C |
| ATOM | 1801 | CB | PHE | A | 271 | 13.480 | −65.581 | −19.013 | 1.00 | 60.47 | C |
| ATOM | 1802 | CG | PHE | A | 271 | 12.029 | −65.224 | −19.166 | 1.00 | 60.39 | C |
| ATOM | 1803 | CD1 | PHE | A | 271 | 11.653 | −64.051 | −19.811 | 1.00 | 60.01 | C |
| ATOM | 1804 | CE1 | PHE | A | 271 | 10.319 | −63.716 | −19.953 | 1.00 | 59.82 | C |
| ATOM | 1805 | CZ | PHE | A | 271 | 9.341 | −64.546 | −19.432 | 1.00 | 60.43 | C |
| ATOM | 1806 | CE2 | PHE | A | 271 | 9.702 | −65.718 | −18.778 | 1.00 | 60.27 | C |
| ATOM | 1807 | CD2 | PHE | A | 271 | 11.042 | −66.049 | −18.646 | 1.00 | 60.08 | C |
| ATOM | 1808 | C | PHE | A | 271 | 15.826 | −65.375 | −19.839 | 1.00 | 60.56 | C |
| ATOM | 1809 | O | PHE | A | 271 | 16.629 | −64.523 | −19.432 | 1.00 | 60.91 | O |
| ATOM | 1810 | N | GLU | A | 272 | 16.172 | −66.638 | −20.064 | 1.00 | 60.53 | N |
| ATOM | 1811 | CA | GLU | A | 272 | 17.557 | −67.032 | −19.881 | 1.00 | 60.88 | C |
| ATOM | 1812 | CB | GLU | A | 272 | 17.780 | −68.554 | −19.987 | 1.00 | 60.68 | C |
| ATOM | 1813 | CG | GLU | A | 272 | 16.859 | −69.330 | −20.916 | 1.00 | 61.39 | C |
| ATOM | 1814 | CD | GLU | A | 272 | 17.054 | −70.852 | −20.801 | 1.00 | 62.06 | C |
| ATOM | 1815 | OE1 | GLU | A | 272 | 16.525 | −71.608 | −21.655 | 1.00 | 63.50 | O |
| ATOM | 1816 | OE2 | GLU | A | 272 | 17.745 | −71.301 | −19.853 | 1.00 | 64.06 | O |
| ATOM | 1817 | C | GLU | A | 272 | 18.472 | −66.221 | −20.804 | 1.00 | 60.54 | C |
| ATOM | 1818 | O | GLU | A | 272 | 19.500 | −65.707 | −20.365 | 1.00 | 60.78 | O |
| ATOM | 1819 | N | LEU | A | 273 | 18.082 | −66.061 | −22.064 | 1.00 | 60.28 | N |
| ATOM | 1820 | CA | LEU | A | 273 | 18.885 | −65.255 | −22.979 | 1.00 | 60.15 | C |
| ATOM | 1821 | CB | LEU | A | 273 | 18.265 | −65.249 | −24.378 | 1.00 | 60.05 | C |
| ATOM | 1822 | CG | LEU | A | 273 | 19.067 | −66.013 | −25.429 | 1.00 | 59.60 | C |
| ATOM | 1823 | CD1 | LEU | A | 273 | 20.501 | −65.525 | −25.383 | 1.00 | 60.64 | C |
| ATOM | 1824 | CD2 | LEU | A | 273 | 19.023 | −67.492 | −25.195 | 1.00 | 59.04 | C |
| ATOM | 1825 | C | LEU | A | 273 | 19.135 | −63.819 | −22.481 | 1.00 | 60.30 | C |
| ATOM | 1826 | O | LEU | A | 273 | 20.285 | −63.350 | −22.429 | 1.00 | 60.39 | O |
| ATOM | 1827 | N | GLU | A | 274 | 18.059 | −63.128 | −22.114 | 1.00 | 60.35 | N |
| ATOM | 1828 | CA | GLU | A | 274 | 18.162 | −61.747 | −21.656 | 1.00 | 60.63 | C |
| ATOM | 1829 | CB | GLU | A | 274 | 16.774 | −61.173 | −21.378 | 1.00 | 60.61 | C |
| ATOM | 1830 | CG | GLU | A | 274 | 15.841 | −61.133 | −22.589 | 1.00 | 61.23 | C |
| ATOM | 1831 | CD | GLU | A | 274 | 16.125 | −59.970 | −23.531 | 1.00 | 62.07 | C |
| ATOM | 1832 | OE1 | GLU | A | 274 | 15.475 | −58.910 | −23.376 | 1.00 | 62.22 | O |
| ATOM | 1833 | OE2 | GLU | A | 274 | 16.996 | −60.115 | −24.421 | 1.00 | 62.05 | O |
| ATOM | 1834 | C | GLU | A | 274 | 19.025 | −61.672 | −20.402 | 1.00 | 60.74 | C |
| ATOM | 1835 | O | GLU | A | 274 | 19.923 | −60.821 | −20.280 | 1.00 | 60.90 | O |
| ATOM | 1836 | N | GLU | A | 275 | 18.752 | −62.578 | −19.473 | 1.00 | 60.89 | N |
| ATOM | 1837 | CA | GLU | A | 275 | 19.561 | −62.697 | −18.276 | 1.00 | 61.09 | C |
| ATOM | 1838 | CB | GLU | A | 275 | 19.106 | −63.919 | −17.491 | 1.00 | 61.28 | C |
| ATOM | 1839 | CG | GLU | A | 275 | 19.561 | −63.971 | −16.051 | 1.00 | 62.13 | C |
| ATOM | 1840 | CD | GLU | A | 275 | 19.147 | −65.272 | −15.391 | 1.00 | 63.38 | C |
| ATOM | 1841 | OE1 | GLU | A | 275 | 19.938 | −65.821 | −14.587 | 1.00 | 63.96 | O |
| ATOM | 1842 | OE2 | GLU | A | 275 | 18.033 | −65.757 | −15.698 | 1.00 | 63.30 | O |
| ATOM | 1843 | C | GLU | A | 275 | 21.037 | −62.822 | −18.658 | 1.00 | 61.04 | C |
| ATOM | 1844 | O | GLU | A | 275 | 21.896 | −62.138 | −18.102 | 1.00 | 60.86 | O |
| ATOM | 1845 | N | GLN | A | 276 | 21.323 | −63.688 | −19.623 | 1.00 | 61.18 | N |
| ATOM | 1846 | CA | GLN | A | 276 | 22.689 | −63.863 | −20.110 | 1.00 | 61.56 | C |
| ATOM | 1847 | CB | GLN | A | 276 | 22.749 | −64.934 | −21.207 | 1.00 | 61.86 | C |
| ATOM | 1848 | CG | GLN | A | 276 | 22.828 | −66.357 | −20.681 | 1.00 | 63.52 | C |
| ATOM | 1849 | CD | GLN | A | 276 | 24.095 | −66.593 | −19.875 | 1.00 | 66.43 | C |
| ATOM | 1850 | OE1 | GLN | A | 276 | 25.197 | −66.659 | −20.431 | 1.00 | 67.98 | O |
| ATOM | 1851 | NE2 | GLN | A | 276 | 23.947 | −66.718 | −18.557 | 1.00 | 67.10 | N |
| ATOM | 1852 | C | GLN | A | 276 | 23.282 | −62.550 | −20.618 | 1.00 | 61.36 | C |
| ATOM | 1853 | O | GLN | A | 276 | 24.414 | −62.196 | −20.269 | 1.00 | 61.33 | O |
| ATOM | 1854 | N | ASP | A | 277 | 22.523 | −61.827 | −21.437 | 1.00 | 61.08 | N |
| ATOM | 1855 | CA | ASP | A | 277 | 23.030 | −60.572 | −21.987 | 1.00 | 61.12 | C |
| ATOM | 1856 | CB | ASP | A | 277 | 22.044 | −59.973 | −22.993 | 1.00 | 61.25 | C |
| ATOM | 1857 | CG | ASP | A | 277 | 21.989 | −60.757 | −24.307 | 1.00 | 62.18 | C |
| ATOM | 1858 | OD1 | ASP | A | 277 | 22.708 | −61.774 | −24.444 | 1.00 | 63.05 | O |
| ATOM | 1859 | OD2 | ASP | A | 277 | 21.223 | −60.356 | −25.213 | 1.00 | 63.07 | O |
| ATOM | 1860 | C | ASP | A | 277 | 23.366 | −59.576 | −20.873 | 1.00 | 60.97 | C |
| ATOM | 1861 | O | ASP | A | 277 | 24.484 | −59.023 | −20.818 | 1.00 | 61.14 | O |
| ATOM | 1862 | N | LEU | A | 278 | 22.411 | −59.359 | −19.969 | 1.00 | 60.75 | N |
| ATOM | 1863 | CA | LEU | A | 278 | 22.657 | −58.435 | −18.859 | 1.00 | 60.28 | C |
| ATOM | 1864 | CB | LEU | A | 278 | 21.412 | −58.273 | −17.973 | 1.00 | 60.28 | C |
| ATOM | 1865 | CG | LEU | A | 278 | 21.560 | −57.508 | −16.651 | 1.00 | 59.63 | C |
| ATOM | 1866 | CD1 | LEU | A | 278 | 21.808 | −56.034 | −16.902 | 1.00 | 58.86 | C |
| ATOM | 1867 | CD2 | LEU | A | 278 | 20.334 | −57.694 | −15.776 | 1.00 | 59.64 | C |
| ATOM | 1868 | C | LEU | A | 278 | 23.860 | −58.894 | −18.034 | 1.00 | 60.34 | C |
| ATOM | 1869 | O | LEU | A | 278 | 24.730 | −58.091 | −17.684 | 1.00 | 60.18 | O |
| ATOM | 1870 | N | PHE | A | 279 | 23.916 | −60.190 | −17.735 | 1.00 | 60.47 | N |
| ATOM | 1871 | CA | PHE | A | 279 | 25.030 | −60.706 | −16.950 | 1.00 | 60.57 | C |
| ATOM | 1872 | CB | PHE | A | 279 | 24.897 | −62.197 | −16.662 | 1.00 | 60.52 | C |
| ATOM | 1873 | CG | PHE | A | 279 | 23.881 | −62.511 | −15.618 | 1.00 | 60.50 | C |
| ATOM | 1874 | CD1 | PHE | A | 279 | 23.466 | −61.533 | −14.729 | 1.00 | 60.16 | C |
| ATOM | 1875 | CE1 | PHE | A | 279 | 22.523 | −61.808 | −13.766 | 1.00 | 60.38 | C |
| ATOM | 1876 | CZ | PHE | A | 279 | 21.992 | −63.075 | −13.670 | 1.00 | 60.80 | C |
| ATOM | 1877 | CE2 | PHE | A | 279 | 22.408 | −64.068 | −14.548 | 1.00 | 60.94 | C |
| ATOM | 1878 | CD2 | PHE | A | 279 | 23.346 | −63.783 | −15.514 | 1.00 | 60.60 | C |
| ATOM | 1879 | C | PHE | A | 279 | 26.350 | −60.418 | −17.630 | 1.00 | 60.72 | C |
| ATOM | 1880 | O | PHE | A | 279 | 27.261 | −59.888 | −17.007 | 1.00 | 60.64 | O |
| ATOM | 1881 | N | ARG | A | 280 | 26.462 | −60.758 | −18.908 | 1.00 | 60.96 | N |
| ATOM | 1882 | CA | ARG | A | 280 | 27.662 | −60.380 | −19.633 | 1.00 | 61.16 | C |
| ATOM | 1883 | CB | ARG | A | 280 | 27.549 | −60.714 | −21.123 | 1.00 | 61.05 | C |
| ATOM | 1884 | CG | ARG | A | 280 | 28.208 | −62.041 | −21.488 | 1.00 | 61.56 | C |
| ATOM | 1885 | CD | ARG | A | 280 | 28.096 | −62.359 | −22.976 | 1.00 | 61.94 | C |
| ATOM | 1886 | NE | ARG | A | 280 | 26.969 | −63.243 | −23.280 | 1.00 | 63.84 | N |
| ATOM | 1887 | CZ | ARG | A | 280 | 25.713 | −62.833 | −23.459 | 1.00 | 64.28 | C |
| ATOM | 1888 | NH1 | ARG | A | 280 | 25.410 | −61.542 | −23.363 | 1.00 | 64.38 | N |
| ATOM | 1889 | NH2 | ARG | A | 280 | 24.759 | −63.718 | −23.733 | 1.00 | 64.17 | N |
| ATOM | 1890 | C | ARG | A | 280 | 27.951 | −58.895 | −19.396 | 1.00 | 61.05 | C |
| ATOM | 1891 | O | ARG | A | 280 | 29.071 | −58.525 | −19.027 | 1.00 | 60.89 | O |
| ATOM | 1892 | N | ASP | A | 281 | 26.932 | −58.053 | −19.561 | 1.00 | 61.18 | N |
| ATOM | 1893 | CA | ASP | A | 281 | 27.123 | −56.605 | −19.389 | 1.00 | 61.26 | C |
| ATOM | 1894 | CB | ASP | A | 281 | 25.814 | −55.846 | −19.642 | 1.00 | 61.67 | C |
| ATOM | 1895 | CG | ASP | A | 281 | 26.023 | −54.555 | −20.433 | 1.00 | 62.92 | C |
| ATOM | 1896 | OD1 | ASP | A | 281 | 27.161 | −54.026 | −20.453 | 1.00 | 63.95 | O |
| ATOM | 1897 | OD2 | ASP | A | 281 | 25.041 | −54.076 | −21.048 | 1.00 | 64.41 | O |
| ATOM | 1898 | C | ASP | A | 281 | 27.700 | −56.222 | −18.020 | 1.00 | 60.84 | C |
| ATOM | 1899 | O | ASP | A | 281 | 28.646 | −55.446 | −17.943 | 1.00 | 60.76 | O |
| ATOM | 1900 | N | ILE | A | 282 | 27.125 | −56.763 | −16.948 | 1.00 | 60.54 | N |
| ATOM | 1901 | CA | ILE | A | 282 | 27.616 | −56.507 | −15.589 | 1.00 | 60.21 | C |
| ATOM | 1902 | CB | ILE | A | 282 | 26.726 | −57.185 | −14.534 | 1.00 | 60.12 | C |
| ATOM | 1903 | CG1 | ILE | A | 282 | 25.316 | −56.592 | −14.547 | 1.00 | 60.23 | C |
| ATOM | 1904 | CD1 | ILE | A | 282 | 24.299 | −57.415 | −13.778 | 1.00 | 59.99 | C |
| ATOM | 1905 | CG2 | ILE | A | 282 | 27.357 | −57.075 | −13.150 | 1.00 | 59.55 | C |
| ATOM | 1906 | C | ILE | A | 282 | 29.009 | −57.077 | −15.409 | 1.00 | 60.29 | C |
| ATOM | 1907 | O | ILE | A | 282 | 29.945 | −56.371 | −15.057 | 1.00 | 60.29 | O |
| ATOM | 1908 | N | GLN | A | 283 | 29.128 | −58.375 | −15.651 | 1.00 | 60.51 | N |
| ATOM | 1909 | CA | GLN | A | 283 | 30.374 | −59.096 | −15.479 | 1.00 | 60.54 | C |
| ATOM | 1910 | CB | GLN | A | 283 | 30.218 | −60.538 | −15.964 | 1.00 | 60.59 | C |
| ATOM | 1911 | CG | GLN | A | 283 | 31.250 | −61.500 | −15.404 | 1.00 | 61.30 | C |
| ATOM | 1912 | CD | GLN | A | 283 | 32.334 | −61.830 | −16.404 | 1.00 | 62.16 | C |
| ATOM | 1913 | OE1 | GLN | A | 283 | 33.515 | −61.936 | −16.054 | 1.00 | 62.37 | O |
| ATOM | 1914 | NE2 | GLN | A | 283 | 31.938 | −61.995 | −17.664 | 1.00 | 62.82 | N |
| ATOM | 1915 | C | GLN | A | 283 | 31.498 | −58.403 | −16.223 | 1.00 | 60.41 | C |
| ATOM | 1916 | O | GLN | A | 283 | 32.660 | −58.514 | −15.838 | 1.00 | 60.58 | O |
| ATOM | 1917 | N | GLY | A | 284 | 31.145 | −57.670 | −17.275 | 1.00 | 60.32 | N |
| ATOM | 1918 | CA | GLY | A | 284 | 32.142 | −56.993 | −18.098 | 1.00 | 59.90 | C |
| ATOM | 1919 | C | GLY | A | 284 | 32.647 | −55.656 | −17.576 | 1.00 | 59.63 | C |
| ATOM | 1920 | O | GLY | A | 284 | 33.536 | −55.052 | −18.175 | 1.00 | 59.69 | O |
| ATOM | 1921 | N | LEU | A | 285 | 32.096 | −55.194 | −16.458 | 1.00 | 59.39 | N |
| ATOM | 1922 | CA | LEU | A | 285 | 32.397 | −53.848 | −15.950 | 1.00 | 59.12 | C |
| ATOM | 1923 | CB | LEU | A | 285 | 31.494 | −53.498 | −14.761 | 1.00 | 58.97 | C |
| ATOM | 1924 | CG | LEU | A | 285 | 30.009 | −53.305 | −15.056 | 1.00 | 58.44 | C |
| ATOM | 1925 | CD1 | LEU | A | 285 | 29.291 | −52.805 | −13.819 | 1.00 | 58.10 | C |
| ATOM | 1926 | CD2 | LEU | A | 285 | 29.822 | −52.345 | −16.210 | 1.00 | 58.23 | C |
| ATOM | 1927 | C | LEU | A | 285 | 33.864 | −53.581 | −15.588 | 1.00 | 59.12 | C |
| ATOM | 1928 | O | LEU | A | 285 | 34.466 | −52.638 | −16.110 | 1.00 | 58.98 | O |
| ATOM | 1929 | N | PRO | A | 286 | 34.444 | −54.409 | −14.698 | 1.00 | 59.17 | N |
| ATOM | 1930 | CA | PRO | A | 286 | 35.792 | −54.145 | −14.203 | 1.00 | 59.19 | C |
| ATOM | 1931 | CB | PRO | A | 286 | 36.075 | −55.339 | −13.288 | 1.00 | 59.12 | C |
| ATOM | 1932 | CG | PRO | A | 286 | 34.744 | −55.870 | −12.925 | 1.00 | 59.19 | C |
| ATOM | 1933 | CD | PRO | A | 286 | 33.888 | −55.648 | −14.127 | 1.00 | 59.17 | C |
| ATOM | 1934 | C | PRO | A | 286 | 36.789 | −54.132 | −15.342 | 1.00 | 59.26 | C |
| ATOM | 1935 | O | PRO | A | 286 | 37.746 | −53.357 | −15.323 | 1.00 | 59.19 | O |
| ATOM | 1936 | N | ARG | A | 287 | 36.553 | −54.989 | −16.331 | 1.00 | 59.46 | N |
| ATOM | 1937 | CA | ARG | A | 287 | 37.412 | −55.057 | −17.499 | 1.00 | 59.64 | C |
| ATOM | 1938 | CB | ARG | A | 287 | 36.797 | −55.921 | −18.592 | 1.00 | 59.76 | C |
| ATOM | 1939 | CG | ARG | A | 287 | 37.768 | −56.326 | −19.690 | 1.00 | 60.10 | C |
| ATOM | 1940 | CD | ARG | A | 287 | 37.147 | −57.427 | −20.510 | 1.00 | 61.21 | C |
| ATOM | 1941 | NE | ARG | A | 287 | 36.470 | −58.384 | −19.637 | 1.00 | 62.03 | N |
| ATOM | 1942 | CZ | ARG | A | 287 | 35.399 | −59.089 | −19.984 | 1.00 | 62.52 | C |
| ATOM | 1943 | NH1 | ARG | A | 287 | 34.852 | −59.933 | −19.118 | 1.00 | 62.89 | N |
| ATOM | 1944 | NH2 | ARG | A | 287 | 34.871 | −58.943 | −21.192 | 1.00 | 62.75 | N |
| ATOM | 1945 | C | ARG | A | 287 | 37.685 | −53.659 | −18.017 | 1.00 | 59.65 | C |
| ATOM | 1946 | O | ARG | A | 287 | 38.790 | −53.378 | −18.474 | 1.00 | 59.81 | O |
| ATOM | 1947 | N | HIS | A | 288 | 36.694 | −52.772 | −17.946 | 1.00 | 59.63 | N |
| ATOM | 1948 | CA | HIS | A | 288 | 37.017 | −51.366 | −18.161 | 1.00 | 59.67 | C |
| ATOM | 1949 | CB | HIS | A | 288 | 37.330 | −51.042 | −19.616 | 1.00 | 59.99 | C |
| ATOM | 1950 | CG | HIS | A | 288 | 38.788 | −50.816 | −19.848 | 1.00 | 61.01 | C |
| ATOM | 1951 | ND1 | HIS | A | 288 | 39.584 | −50.164 | −18.929 | 1.00 | 61.71 | N |
| ATOM | 1952 | CE1 | HIS | A | 288 | 40.826 | −50.118 | −19.381 | 1.00 | 62.63 | C |
| ATOM | 1953 | NE2 | HIS | A | 288 | 40.864 | −50.719 | −20.558 | 1.00 | 62.85 | N |
| ATOM | 1954 | CD2 | HIS | A | 288 | 39.603 | −51.171 | −20.870 | 1.00 | 62.15 | C |
| ATOM | 1955 | C | HIS | A | 288 | 36.213 | −50.263 | −17.500 | 1.00 | 59.26 | C |
| ATOM | 1956 | O | HIS | A | 288 | 35.213 | −49.774 | −18.027 | 1.00 | 59.07 | O |
| ATOM | 1957 | N | ALA | A | 289 | 36.721 | −49.867 | −16.340 | 1.00 | 58.88 | N |
| ATOM | 1958 | CA | ALA | A | 289 | 36.302 | −48.660 | −15.668 | 1.00 | 58.55 | C |
| ATOM | 1959 | CB | ALA | A | 289 | 36.632 | −48.746 | −14.182 | 1.00 | 58.66 | C |
| ATOM | 1960 | C | ALA | A | 289 | 37.013 | −47.468 | −16.301 | 1.00 | 58.24 | C |
| ATOM | 1961 | O | ALA | A | 289 | 36.464 | −46.372 | −16.359 | 1.00 | 58.03 | O |
| ATOM | 1962 | N | ALA | A | 290 | 38.233 | −47.688 | −16.781 | 1.00 | 57.82 | N |
| ATOM | 1963 | CA | ALA | A | 290 | 39.024 | −46.609 | −17.358 | 1.00 | 57.55 | C |
| ATOM | 1964 | CB | ALA | A | 290 | 40.307 | −47.154 | −17.938 | 1.00 | 57.56 | C |
| ATOM | 1965 | C | ALA | A | 290 | 38.252 | −45.822 | −18.416 | 1.00 | 57.41 | C |
| ATOM | 1966 | O | ALA | A | 290 | 38.284 | −44.587 | −18.437 | 1.00 | 57.25 | O |
| ATOM | 1967 | N | LEU | A | 291 | 37.557 | −46.542 | −19.289 | 1.00 | 57.29 | N |
| ATOM | 1968 | CA | LEU | A | 291 | 36.796 | −45.916 | −20.362 | 1.00 | 57.23 | C |
| ATOM | 1969 | CB | LEU | A | 291 | 36.243 | −46.984 | −21.308 | 1.00 | 57.15 | C |
| ATOM | 1970 | CG | LEU | A | 291 | 36.111 | −46.651 | −22.796 | 1.00 | 56.86 | C |
| ATOM | 1971 | CD1 | LEU | A | 291 | 35.366 | −47.763 | −23.507 | 1.00 | 56.79 | C |
| ATOM | 1972 | CD2 | LEU | A | 291 | 35.407 | −45.328 | −23.016 | 1.00 | 57.08 | C |
| ATOM | 1973 | C | LEU | A | 291 | 35.658 | −45.059 | −19.802 | 1.00 | 57.31 | C |
| ATOM | 1974 | O | LEU | A | 291 | 35.502 | −43.894 | −20.180 | 1.00 | 57.24 | O |
| ATOM | 1975 | N | ARG | A | 292 | 34.866 | −45.629 | −18.897 | 1.00 | 57.43 | N |
| ATOM | 1976 | CA | ARG | A | 292 | 33.724 | −44.901 | −18.343 | 1.00 | 57.57 | C |
| ATOM | 1977 | CB | ARG | A | 292 | 32.790 | −45.826 | −17.545 | 1.00 | 57.62 | C |
| ATOM | 1978 | CG | ARG | A | 292 | 33.064 | −45.912 | −16.049 | 1.00 | 57.76 | C |
| ATOM | 1979 | CD | ARG | A | 292 | 32.064 | −45.093 | −15.239 | 1.00 | 57.94 | C |
| ATOM | 1980 | NE | ARG | A | 292 | 32.428 | −45.049 | −13.823 | 1.00 | 58.09 | N |
| ATOM | 1981 | CZ | ARG | A | 292 | 32.935 | −43.984 | −13.209 | 1.00 | 57.92 | C |
| ATOM | 1982 | NH1 | ARG | A | 292 | 33.241 | −44.043 | −11.923 | 1.00 | 58.28 | N |
| ATOM | 1983 | NH2 | ARG | A | 292 | 33.133 | −42.855 | −13.874 | 1.00 | 58.17 | N |
| ATOM | 1984 | C | ARG | A | 292 | 34.204 | −43.718 | −17.508 | 1.00 | 57.46 | C |
| ATOM | 1985 | O | ARG | A | 292 | 33.588 | −42.648 | −17.520 | 1.00 | 57.56 | O |
| ATOM | 1986 | N | LYS | A | 293 | 35.312 | −43.912 | −16.802 | 1.00 | 57.36 | N |
| ATOM | 1987 | CA | LYS | A | 293 | 35.942 | −42.831 | −16.067 | 1.00 | 57.19 | C |
| ATOM | 1988 | CB | LYS | A | 293 | 37.186 | −43.323 | −15.330 | 1.00 | 57.19 | C |
| ATOM | 1989 | CG | LYS | A | 293 | 36.886 | −44.029 | −14.020 | 1.00 | 57.17 | C |
| ATOM | 1990 | CD | LYS | A | 293 | 38.050 | −44.901 | −13.578 | 1.00 | 57.02 | C |
| ATOM | 1991 | CE | LYS | A | 293 | 37.757 | −45.577 | −12.248 | 1.00 | 57.19 | C |
| ATOM | 1992 | NZ | LYS | A | 293 | 38.749 | −46.645 | −11.944 | 1.00 | 57.34 | N |
| ATOM | 1993 | C | LYS | A | 293 | 36.304 | −41.729 | −17.043 | 1.00 | 57.09 | C |
| ATOM | 1994 | O | LYS | A | 293 | 35.865 | −40.588 | −16.892 | 1.00 | 57.11 | O |
| ATOM | 1995 | N | LEU | A | 294 | 37.091 | −42.079 | −18.055 | 1.00 | 56.95 | N |
| ATOM | 1996 | CA | LEU | A | 294 | 37.458 | −41.121 | −19.083 | 1.00 | 56.84 | C |
| ATOM | 1997 | CB | LEU | A | 294 | 38.123 | −41.836 | −20.259 | 1.00 | 56.79 | C |
| ATOM | 1998 | CG | LEU | A | 294 | 38.441 | −40.974 | −21.483 | 1.00 | 56.79 | C |
| ATOM | 1999 | CD1 | LEU | A | 294 | 39.335 | −39.809 | −21.097 | 1.00 | 57.30 | C |
| ATOM | 2000 | CD2 | LEU | A | 294 | 39.095 | −41.802 | −22.568 | 1.00 | 56.83 | C |
| ATOM | 2001 | C | LEU | A | 294 | 36.226 | −40.348 | −19.550 | 1.00 | 56.79 | C |
| ATOM | 2002 | O | LEU | A | 294 | 36.167 | −39.112 | −19.454 | 1.00 | 56.76 | O |
| ATOM | 2003 | N | ASN | A | 295 | 35.236 | −41.084 | −20.039 | 1.00 | 56.85 | N |
| ATOM | 2004 | CA | ASN | A | 295 | 34.003 | −40.469 | −20.527 | 1.00 | 56.94 | C |
| ATOM | 2005 | CB | ASN | A | 295 | 32.999 | −41.549 | −20.922 | 1.00 | 57.00 | C |
| ATOM | 2006 | CG | ASN | A | 295 | 32.915 | −41.708 | −22.436 | 1.00 | 57.02 | C |
| ATOM | 2007 | OD1 | ASN | A | 295 | 33.082 | −42.815 | −22.972 | 1.00 | 56.80 | O |
| ATOM | 2008 | ND2 | ASN | A | 295 | 32.656 | −40.594 | −23.140 | 1.00 | 57.22 | N |
| ATOM | 2009 | C | ASN | A | 295 | 33.355 | −39.494 | −19.545 | 1.00 | 56.98 | C |
| ATOM | 2010 | O | ASN | A | 295 | 33.029 | −38.355 | −19.897 | 1.00 | 56.90 | O |
| ATOM | 2011 | N | ASP | A | 296 | 33.163 | −39.939 | −18.311 | 1.00 | 57.20 | N |
| ATOM | 2012 | CA | ASP | A | 296 | 32.544 | −39.075 | −17.319 | 1.00 | 57.27 | C |
| ATOM | 2013 | CB | ASP | A | 296 | 32.240 | −39.854 | −16.040 | 1.00 | 57.49 | C |
| ATOM | 2014 | CG | ASP | A | 296 | 31.232 | −40.978 | −16.270 | 1.00 | 58.34 | C |
| ATOM | 2015 | OD1 | ASP | A | 296 | 31.236 | −41.950 | −15.485 | 1.00 | 59.28 | O |
| ATOM | 2016 | OD2 | ASP | A | 296 | 30.441 | −40.898 | −17.241 | 1.00 | 59.32 | O |
| ATOM | 2017 | C | ASP | A | 296 | 33.409 | −37.844 | −17.057 | 1.00 | 57.08 | C |
| ATOM | 2018 | O | ASP | A | 296 | 32.886 | −36.745 | −16.834 | 1.00 | 56.96 | O |
| ATOM | 2019 | N | LEU | A | 297 | 34.728 | −38.027 | −17.115 | 1.00 | 56.92 | N |
| ATOM | 2020 | CA | LEU | A | 297 | 35.652 | −36.901 | −17.027 | 1.00 | 56.71 | C |
| ATOM | 2021 | CB | LEU | A | 297 | 37.110 | −37.360 | −17.104 | 1.00 | 56.70 | C |
| ATOM | 2022 | CG | LEU | A | 297 | 38.145 | −36.289 | −16.736 | 1.00 | 57.00 | C |
| ATOM | 2023 | CD1 | LEU | A | 297 | 38.338 | −36.212 | −15.230 | 1.00 | 57.18 | C |
| ATOM | 2024 | CD2 | LEU | A | 297 | 39.486 | −36.538 | −17.417 | 1.00 | 57.32 | C |
| ATOM | 2025 | C | LEU | A | 297 | 35.357 | −35.904 | −18.140 | 1.00 | 56.57 | C |
| ATOM | 2026 | O | LEU | A | 297 | 35.223 | −34.709 | −17.882 | 1.00 | 56.57 | O |
| ATOM | 2027 | N | VAL | A | 298 | 35.246 | −36.394 | −19.374 | 1.00 | 56.40 | N |
| ATOM | 2028 | CA | VAL | A | 298 | 34.872 | −35.528 | −20.498 | 1.00 | 56.20 | C |
| ATOM | 2029 | CB | VAL | A | 298 | 34.701 | −36.323 | −21.801 | 1.00 | 56.10 | C |
| ATOM | 2030 | CG1 | VAL | A | 298 | 34.213 | −35.414 | −22.920 | 1.00 | 55.78 | C |
| ATOM | 2031 | CG2 | VAL | A | 298 | 35.994 | −37.011 | −22.183 | 1.00 | 55.98 | C |
| ATOM | 2032 | C | VAL | A | 298 | 33.562 | −34.781 | −20.239 | 1.00 | 56.34 | C |
| ATOM | 2033 | O | VAL | A | 298 | 33.490 | −33.550 | −20.372 | 1.00 | 56.51 | O |
| ATOM | 2034 | N | LYS | A | 299 | 32.523 | −35.532 | −19.880 | 1.00 | 56.48 | N |
| ATOM | 2035 | CA | LYS | A | 299 | 31.207 | −34.944 | −19.649 | 1.00 | 56.48 | C |
| ATOM | 2036 | CB | LYS | A | 299 | 30.223 | −36.009 | −19.163 | 1.00 | 56.50 | C |
| ATOM | 2037 | CG | LYS | A | 299 | 29.928 | −37.118 | −20.171 | 1.00 | 56.54 | C |
| ATOM | 2038 | CD | LYS | A | 299 | 29.057 | −38.204 | −19.543 | 1.00 | 56.73 | C |
| ATOM | 2039 | CE | LYS | A | 299 | 28.654 | −39.272 | −20.554 | 1.00 | 57.33 | C |
| ATOM | 2040 | NZ | LYS | A | 299 | 27.703 | −38.749 | −21.571 | 1.00 | 57.04 | N |
| ATOM | 2041 | C | LYS | A | 299 | 31.312 | −33.824 | −18.623 | 1.00 | 56.44 | C |
| ATOM | 2042 | O | LYS | A | 299 | 30.872 | −32.676 | −18.860 | 1.00 | 56.62 | O |
| ATOM | 2043 | N | ARG | A | 300 | 31.910 | −34.158 | −17.482 | 1.00 | 56.46 | N |
| ATOM | 2044 | CA | ARG | A | 300 | 32.147 | −33.159 | −16.456 | 1.00 | 56.31 | C |
| ATOM | 2045 | CB | ARG | A | 300 | 32.985 | −33.720 | −15.307 | 1.00 | 56.27 | C |
| ATOM | 2046 | CG | ARG | A | 300 | 33.478 | −32.651 | −14.337 | 1.00 | 56.17 | C |
| ATOM | 2047 | CD | ARG | A | 300 | 34.274 | −33.246 | −13.191 | 1.00 | 56.14 | C |
| ATOM | 2048 | NE | ARG | A | 300 | 33.429 | −33.563 | −12.047 | 1.00 | 56.16 | N |
| ATOM | 2049 | CZ | ARG | A | 300 | 33.530 | −32.970 | −10.862 | 1.00 | 56.53 | C |
| ATOM | 2050 | NH1 | ARG | A | 300 | 32.722 | −33.317 | −9.870 | 1.00 | 56.30 | N |
| ATOM | 2051 | NH2 | ARG | A | 300 | 34.450 | −32.037 | −10.665 | 1.00 | 56.98 | N |
| ATOM | 2052 | C | ARG | A | 300 | 32.834 | −31.952 | −17.081 | 1.00 | 56.37 | C |
| ATOM | 2053 | O | ARG | A | 300 | 32.329 | −30.834 | −16.996 | 1.00 | 56.39 | O |
| ATOM | 2054 | N | ALA | A | 301 | 33.973 | −32.186 | −17.729 | 1.00 | 56.45 | N |
| ATOM | 2055 | CA | ALA | A | 301 | 34.714 | −31.106 | −18.374 | 1.00 | 56.41 | C |
| ATOM | 2056 | CB | ALA | A | 301 | 35.758 | −31.660 | −19.331 | 1.00 | 56.29 | C |
| ATOM | 2057 | C | ALA | A | 301 | 33.761 | −30.172 | −19.104 | 1.00 | 56.46 | C |
| ATOM | 2058 | O | ALA | A | 301 | 33.722 | −28.971 | −18.821 | 1.00 | 56.47 | O |
| ATOM | 2059 | N | ARG | A | 302 | 32.980 | −30.728 | −20.025 | 1.00 | 56.55 | N |
| ATOM | 2060 | CA | ARG | A | 302 | 32.035 | −29.923 | −20.808 | 1.00 | 56.77 | C |
| ATOM | 2061 | CB | ARG | A | 302 | 31.260 | −30.818 | −21.777 | 1.00 | 56.86 | C |
| ATOM | 2062 | CG | ARG | A | 302 | 32.166 | −31.414 | −22.840 | 1.00 | 57.73 | C |
| ATOM | 2063 | CD | ARG | A | 302 | 31.637 | −32.710 | −23.404 | 1.00 | 59.51 | C |
| ATOM | 2064 | NE | ARG | A | 302 | 30.704 | −32.490 | −24.503 | 1.00 | 61.12 | N |
| ATOM | 2065 | CZ | ARG | A | 302 | 29.383 | −32.597 | −24.393 | 1.00 | 62.12 | C |
| ATOM | 2066 | NH1 | ARG | A | 302 | 28.831 | −32.927 | −23.228 | 1.00 | 62.16 | N |
| ATOM | 2067 | NH2 | ARG | A | 302 | 28.612 | −32.381 | −25.452 | 1.00 | 62.71 | N |
| ATOM | 2068 | C | ARG | A | 302 | 31.093 | −29.087 | −19.932 | 1.00 | 56.68 | C |
| ATOM | 2069 | O | ARG | A | 302 | 30.924 | −27.862 | −20.149 | 1.00 | 56.68 | O |
| ATOM | 2070 | N | LEU | A | 303 | 30.503 | −29.735 | −18.927 | 1.00 | 56.67 | N |
| ATOM | 2071 | CA | LEU | A | 303 | 29.663 | −28.990 | −17.981 | 1.00 | 56.63 | C |
| ATOM | 2072 | CB | LEU | A | 303 | 29.083 | −29.913 | −16.910 | 1.00 | 56.59 | C |
| ATOM | 2073 | CG | LEU | A | 303 | 28.182 | −29.195 | −15.902 | 1.00 | 56.67 | C |
| ATOM | 2074 | CD1 | LEU | A | 303 | 26.913 | −28.699 | −16.582 | 1.00 | 56.50 | C |
| ATOM | 2075 | CD2 | LEU | A | 303 | 27.844 | −30.106 | −14.733 | 1.00 | 57.07 | C |
| ATOM | 2076 | C | LEU | A | 303 | 30.433 | −27.832 | −17.323 | 1.00 | 56.59 | C |
| ATOM | 2077 | O | LEU | A | 303 | 29.971 | −26.677 | −17.307 | 1.00 | 56.69 | O |
| ATOM | 2078 | N | VAL | A | 304 | 31.606 | −28.149 | −16.781 | 1.00 | 56.40 | N |
| ATOM | 2079 | CA | VAL | A | 304 | 32.482 | −27.143 | −16.199 | 1.00 | 56.15 | C |
| ATOM | 2080 | CB | VAL | A | 304 | 33.862 | −27.728 | −15.855 | 1.00 | 56.03 | C |
| ATOM | 2081 | CG1 | VAL | A | 304 | 34.801 | −26.633 | −15.389 | 1.00 | 55.85 | C |
| ATOM | 2082 | CG2 | VAL | A | 304 | 33.728 | −28.800 | −14.797 | 1.00 | 55.67 | C |
| ATOM | 2083 | C | VAL | A | 304 | 32.655 | −25.971 | −17.159 | 1.00 | 56.22 | C |
| ATOM | 2084 | O | VAL | A | 304 | 32.319 | −24.832 | −16.825 | 1.00 | 56.31 | O |
| ATOM | 2085 | N | ARG | A | 305 | 33.166 | −26.249 | −18.356 | 1.00 | 56.24 | N |
| ATOM | 2086 | CA | ARG | A | 305 | 33.364 | −25.195 | −19.351 | 1.00 | 56.28 | C |
| ATOM | 2087 | CB | ARG | A | 305 | 33.760 | −25.783 | −20.714 | 1.00 | 56.25 | C |
| ATOM | 2088 | CG | ARG | A | 305 | 33.325 | −24.961 | −21.919 | 1.00 | 56.53 | C |
| ATOM | 2089 | CD | ARG | A | 305 | 34.485 | −24.659 | −22.872 | 1.00 | 57.09 | C |
| ATOM | 2090 | NE | ARG | A | 305 | 34.903 | −25.757 | −23.746 | 1.00 | 57.25 | N |
| ATOM | 2091 | CZ | ARG | A | 305 | 34.124 | −26.746 | −24.174 | 1.00 | 57.93 | C |
| ATOM | 2092 | NH1 | ARG | A | 305 | 34.632 | −27.680 | −24.968 | 1.00 | 57.81 | N |
| ATOM | 2093 | NH2 | ARG | A | 305 | 32.845 | −26.809 | −23.820 | 1.00 | 58.76 | N |
| ATOM | 2094 | C | ARG | A | 305 | 32.126 | −24.303 | −19.448 | 1.00 | 56.28 | C |
| ATOM | 2095 | O | ARG | A | 305 | 32.223 | −23.058 | −19.357 | 1.00 | 56.39 | O |
| ATOM | 2096 | N | VAL | A | 306 | 30.956 | −24.931 | −19.589 | 1.00 | 56.35 | N |
| ATOM | 2097 | CA | VAL | A | 306 | 29.727 | −24.136 | −19.630 | 1.00 | 56.27 | C |
| ATOM | 2098 | CB | VAL | A | 306 | 28.487 | −25.017 | −19.682 | 1.00 | 56.15 | C |
| ATOM | 2099 | CG1 | VAL | A | 306 | 27.267 | −24.162 | −19.959 | 1.00 | 56.18 | C |
| ATOM | 2100 | CG2 | VAL | A | 306 | 28.652 | −26.085 | −20.738 | 1.00 | 56.17 | C |
| ATOM | 2101 | C | VAL | A | 306 | 29.608 | −23.217 | −18.409 | 1.00 | 56.36 | C |
| ATOM | 2102 | O | VAL | A | 306 | 29.536 | −21.983 | −18.532 | 1.00 | 56.51 | O |
| ATOM | 2103 | N | HIS | A | 307 | 29.588 | −23.822 | −17.227 | 1.00 | 56.41 | N |
| ATOM | 2104 | CA | HIS | A | 307 | 29.471 | −23.049 | −15.993 | 1.00 | 56.44 | C |
| ATOM | 2105 | CB | HIS | A | 307 | 29.743 | −23.942 | −14.783 | 1.00 | 56.45 | C |
| ATOM | 2106 | CG | HIS | A | 307 | 29.652 | −23.225 | −13.474 | 1.00 | 55.91 | C |
| ATOM | 2107 | ND1 | HIS | A | 307 | 28.449 | −22.849 | −12.914 | 1.00 | 55.76 | N |
| ATOM | 2108 | CE1 | HIS | A | 307 | 28.672 | −22.245 | −11.760 | 1.00 | 55.92 | C |
| ATOM | 2109 | NE2 | HIS | A | 307 | 29.976 | −22.221 | −11.548 | 1.00 | 56.18 | N |
| ATOM | 2110 | CD2 | HIS | A | 307 | 30.612 | −22.827 | −12.606 | 1.00 | 55.83 | C |
| ATOM | 2111 | C | HIS | A | 307 | 30.427 | −21.856 | −15.981 | 1.00 | 56.54 | C |
| ATOM | 2112 | O | HIS | A | 307 | 30.023 | −20.712 | −15.705 | 1.00 | 56.69 | O |
| ATOM | 2113 | N | ALA | A | 308 | 31.696 | −22.138 | −16.281 | 1.00 | 56.50 | N |
| ATOM | 2114 | CA | ALA | A | 308 | 32.722 | −21.106 | −16.342 | 1.00 | 56.41 | C |
| ATOM | 2115 | CB | ALA | A | 308 | 34.000 | −21.659 | −16.921 | 1.00 | 56.25 | C |
| ATOM | 2116 | C | ALA | A | 308 | 32.211 | −19.972 | −17.198 | 1.00 | 56.41 | C |
| ATOM | 2117 | O | ALA | A | 308 | 32.153 | −18.819 | −16.751 | 1.00 | 56.62 | O |
| ATOM | 2118 | N | TYR | A | 309 | 31.814 | −20.299 | −18.424 | 1.00 | 56.40 | N |
| ATOM | 2119 | CA | TYR | A | 309 | 31.311 | −19.251 | −19.299 | 1.00 | 56.51 | C |
| ATOM | 2120 | CB | TYR | A | 309 | 30.868 | −19.809 | −20.650 | 1.00 | 56.57 | C |
| ATOM | 2121 | CG | TYR | A | 309 | 32.021 | −20.031 | −21.598 | 1.00 | 56.51 | C |
| ATOM | 2122 | CD1 | TYR | A | 309 | 32.161 | −21.225 | −22.287 | 1.00 | 56.48 | C |
| ATOM | 2123 | CE1 | TYR | A | 309 | 33.219 | −21.429 | −23.150 | 1.00 | 56.59 | C |
| ATOM | 2124 | CZ | TYR | A | 309 | 34.159 | −20.436 | −23.323 | 1.00 | 56.61 | C |
| ATOM | 2125 | OH | TYR | A | 309 | 35.215 | −20.639 | −24.178 | 1.00 | 56.68 | O |
| ATOM | 2126 | CE2 | TYR | A | 309 | 34.048 | −19.243 | −22.644 | 1.00 | 56.61 | C |
| ATOM | 2127 | CD2 | TYR | A | 309 | 32.985 | −19.048 | −21.786 | 1.00 | 56.59 | C |
| ATOM | 2128 | C | TYR | A | 309 | 30.193 | −18.455 | −18.632 | 1.00 | 56.61 | C |
| ATOM | 2129 | O | TYR | A | 309 | 30.250 | −17.219 | −18.581 | 1.00 | 56.91 | O |
| ATOM | 2130 | N | ILE | A | 310 | 29.193 | −19.158 | −18.102 | 1.00 | 56.59 | N |
| ATOM | 2131 | CA | ILE | A | 310 | 28.068 | −18.486 | −17.439 | 1.00 | 56.63 | C |
| ATOM | 2132 | CB | ILE | A | 310 | 27.113 | −19.492 | −16.774 | 1.00 | 56.52 | C |
| ATOM | 2133 | CG1 | ILE | A | 310 | 26.552 | −20.463 | −17.809 | 1.00 | 56.47 | C |
| ATOM | 2134 | CD1 | ILE | A | 310 | 25.786 | −21.610 | −17.202 | 1.00 | 56.57 | C |
| ATOM | 2135 | CG2 | ILE | A | 310 | 25.980 | −18.768 | −16.079 | 1.00 | 56.40 | C |
| ATOM | 2136 | C | ILE | A | 310 | 28.523 | −17.463 | −16.387 | 1.00 | 56.78 | C |
| ATOM | 2137 | O | ILE | A | 310 | 28.192 | −16.258 | −16.473 | 1.00 | 57.05 | O |
| ATOM | 2138 | N | ILE | A | 311 | 29.283 | −17.938 | −15.399 | 1.00 | 56.75 | N |
| ATOM | 2139 | CA | ILE | A | 311 | 29.750 | −17.046 | −14.338 | 1.00 | 56.82 | C |
| ATOM | 2140 | CB | ILE | A | 311 | 30.607 | −17.771 | −13.281 | 1.00 | 56.78 | C |
| ATOM | 2141 | CG1 | ILE | A | 311 | 29.743 | −18.232 | −12.109 | 1.00 | 56.78 | C |
| ATOM | 2142 | CD1 | ILE | A | 311 | 28.714 | −19.266 | −12.461 | 1.00 | 57.40 | C |
| ATOM | 2143 | CG2 | ILE | A | 311 | 31.655 | −16.834 | −12.716 | 1.00 | 57.09 | C |
| ATOM | 2144 | C | ILE | A | 311 | 30.532 | −15.882 | −14.932 | 1.00 | 56.87 | C |
| ATOM | 2145 | O | ILE | A | 311 | 30.257 | −14.714 | −14.630 | 1.00 | 56.99 | O |
| ATOM | 2146 | N | SER | A | 312 | 31.495 | −16.205 | −15.792 | 1.00 | 56.92 | N |
| ATOM | 2147 | CA | SER | A | 312 | 32.304 | −15.181 | −16.436 | 1.00 | 57.06 | C |
| ATOM | 2148 | CB | SER | A | 312 | 33.181 | −15.797 | −17.521 | 1.00 | 57.04 | C |
| ATOM | 2149 | OG | SER | A | 312 | 34.124 | −16.687 | −16.952 | 1.00 | 57.03 | O |
| ATOM | 2150 | C | SER | A | 312 | 31.437 | −14.078 | −17.028 | 1.00 | 57.22 | C |
| ATOM | 2151 | O | SER | A | 312 | 31.633 | −12.896 | −16.726 | 1.00 | 57.33 | O |
| ATOM | 2152 | N | TYR | A | 313 | 30.474 | −14.464 | −17.864 | 1.00 | 57.40 | N |
| ATOM | 2153 | CA | TYR | A | 313 | 29.591 | −13.479 | −18.501 | 1.00 | 57.83 | C |
| ATOM | 2154 | CB | TYR | A | 313 | 28.638 | −14.142 | −19.503 | 1.00 | 58.15 | C |
| ATOM | 2155 | CG | TYR | A | 313 | 29.257 | −14.429 | −20.864 | 1.00 | 58.84 | C |
| ATOM | 2156 | CD1 | TYR | A | 313 | 28.576 | −14.120 | −22.041 | 1.00 | 59.36 | C |
| ATOM | 2157 | CE1 | TYR | A | 313 | 29.137 | −14.383 | −23.290 | 1.00 | 59.39 | C |
| ATOM | 2158 | CZ | TYR | A | 313 | 30.395 | −14.957 | −23.368 | 1.00 | 59.16 | C |
| ATOM | 2159 | OH | TYR | A | 313 | 30.957 | −15.219 | −24.597 | 1.00 | 59.00 | O |
| ATOM | 2160 | CE2 | TYR | A | 313 | 31.093 | −15.269 | −22.216 | 1.00 | 59.29 | C |
| ATOM | 2161 | CD2 | TYR | A | 313 | 30.525 | −15.004 | −20.974 | 1.00 | 59.34 | C |
| ATOM | 2162 | C | TYR | A | 313 | 28.815 | −12.657 | −17.471 | 1.00 | 57.75 | C |
| ATOM | 2163 | O | TYR | A | 313 | 28.734 | −11.423 | −17.578 | 1.00 | 57.91 | O |
| ATOM | 2164 | N | LEU | A | 314 | 28.257 | −13.328 | −16.465 | 1.00 | 57.51 | N |
| ATOM | 2165 | CA | LEU | A | 314 | 27.569 | −12.590 | −15.403 | 1.00 | 57.40 | C |
| ATOM | 2166 | CB | LEU | A | 314 | 27.057 | −13.550 | −14.330 | 1.00 | 57.20 | C |
| ATOM | 2167 | CG | LEU | A | 314 | 25.894 | −14.430 | −14.790 | 1.00 | 56.64 | C |
| ATOM | 2168 | CD1 | LEU | A | 314 | 25.739 | −15.648 | −13.905 | 1.00 | 55.97 | C |
| ATOM | 2169 | CD2 | LEU | A | 314 | 24.612 | −13.622 | −14.836 | 1.00 | 56.28 | C |
| ATOM | 2170 | C | LEU | A | 314 | 28.481 | −11.514 | −14.795 | 1.00 | 57.49 | C |
| ATOM | 2171 | O | LEU | A | 314 | 28.138 | −10.316 | −14.753 | 1.00 | 57.84 | O |
| ATOM | 2172 | N | LYS | A | 315 | 29.656 | −11.949 | −14.344 | 1.00 | 57.46 | N |
| ATOM | 2173 | CA | LYS | A | 315 | 30.624 | −11.045 | −13.725 | 1.00 | 57.51 | C |
| ATOM | 2174 | CB | LYS | A | 315 | 31.880 | −11.804 | −13.290 | 1.00 | 57.50 | C |
| ATOM | 2175 | CG | LYS | A | 315 | 33.079 | −10.920 | −12.986 | 1.00 | 57.23 | C |
| ATOM | 2176 | CD | LYS | A | 315 | 32.828 | −10.022 | −11.793 | 1.00 | 57.26 | C |
| ATOM | 2177 | CE | LYS | A | 315 | 34.012 | −9.103 | −11.556 | 1.00 | 57.59 | C |
| ATOM | 2178 | NZ | LYS | A | 315 | 33.958 | −8.446 | −10.218 | 1.00 | 57.78 | N |
| ATOM | 2179 | C | LYS | A | 315 | 31.003 | −9.898 | −14.648 | 1.00 | 57.60 | C |
| ATOM | 2180 | O | LYS | A | 315 | 31.104 | −8.753 | −14.207 | 1.00 | 57.65 | O |
| ATOM | 2181 | N | LYS | A | 316 | 31.217 | −10.202 | −15.925 | 1.00 | 57.67 | N |
| ATOM | 2182 | CA | LYS | A | 316 | 31.571 | −9.156 | −16.880 | 1.00 | 57.96 | C |
| ATOM | 2183 | CB | LYS | A | 316 | 32.094 | −9.747 | −18.198 | 1.00 | 58.04 | C |
| ATOM | 2184 | CG | LYS | A | 316 | 31.033 | −10.014 | −19.266 | 1.00 | 58.13 | C |
| ATOM | 2185 | CD | LYS | A | 316 | 31.633 | −10.748 | −20.463 | 1.00 | 58.14 | C |
| ATOM | 2186 | CE | LYS | A | 316 | 30.559 | −11.212 | −21.441 | 1.00 | 58.37 | C |
| ATOM | 2187 | NZ | LYS | A | 316 | 29.905 | −10.080 | −22.159 | 1.00 | 58.37 | N |
| ATOM | 2188 | C | LYS | A | 316 | 30.386 | −8.223 | −17.128 | 1.00 | 57.99 | C |
| ATOM | 2189 | O | LYS | A | 316 | 30.559 | −7.095 | −17.590 | 1.00 | 57.89 | O |
| ATOM | 2190 | N | GLU | A | 317 | 29.183 | −8.693 | −16.812 | 1.00 | 58.12 | N |
| ATOM | 2191 | CA | GLU | A | 317 | 28.004 | −7.848 | −16.967 | 1.00 | 58.35 | C |
| ATOM | 2192 | CB | GLU | A | 317 | 26.752 | −8.689 | −17.228 | 1.00 | 58.34 | C |
| ATOM | 2193 | CG | GLU | A | 317 | 25.722 | −7.983 | −18.087 | 1.00 | 58.29 | C |
| ATOM | 2194 | CD | GLU | A | 317 | 26.286 | −7.568 | −19.434 | 1.00 | 58.49 | C |
| ATOM | 2195 | OE1 | GLU | A | 317 | 25.892 | −6.495 | −19.943 | 1.00 | 58.78 | O |
| ATOM | 2196 | OE2 | GLU | A | 317 | 27.130 | −8.311 | −19.982 | 1.00 | 58.20 | O |
| ATOM | 2197 | C | GLU | A | 317 | 27.791 | −6.929 | −15.765 | 1.00 | 58.52 | C |
| ATOM | 2198 | O | GLU | A | 317 | 27.312 | −5.806 | −15.919 | 1.00 | 58.43 | O |
| ATOM | 2199 | N | MET | A | 318 | 28.147 | −7.408 | −14.574 | 1.00 | 58.76 | N |
| ATOM | 2200 | CA | MET | A | 318 | 27.957 | −6.621 | −13.339 | 1.00 | 59.38 | C |
| ATOM | 2201 | CB | MET | A | 318 | 28.350 | −7.457 | −12.122 | 1.00 | 59.35 | C |
| ATOM | 2202 | CG | MET | A | 318 | 27.290 | −8.450 | −11.703 | 1.00 | 59.35 | C |
| ATOM | 2203 | SD | MET | A | 318 | 25.852 | −7.627 | −11.000 | 1.00 | 59.21 | S |
| ATOM | 2204 | CE | MET | A | 318 | 26.589 | −6.840 | −9.568 | 1.00 | 59.36 | C |
| ATOM | 2205 | C | MET | A | 318 | 28.694 | −5.273 | −13.292 | 1.00 | 59.81 | C |
| ATOM | 2206 | O | MET | A | 318 | 29.870 | −5.198 | −13.641 | 1.00 | 59.90 | O |
| ATOM | 2207 | N | PRO | A | 319 | 28.001 | −4.208 | −12.835 | 1.00 | 60.27 | N |
| ATOM | 2208 | CA | PRO | A | 319 | 28.570 | −2.859 | −12.719 | 1.00 | 60.66 | C |
| ATOM | 2209 | CB | PRO | A | 319 | 27.330 | −1.968 | −12.653 | 1.00 | 60.60 | C |
| ATOM | 2210 | CG | PRO | A | 319 | 26.311 | −2.817 | −11.979 | 1.00 | 60.41 | C |
| ATOM | 2211 | CD | PRO | A | 319 | 26.597 | −4.248 | −12.386 | 1.00 | 60.27 | C |
| ATOM | 2212 | C | PRO | A | 319 | 29.398 | −2.698 | −11.446 | 1.00 | 61.12 | C |
| ATOM | 2213 | O | PRO | A | 319 | 29.036 | −3.243 | −10.399 | 1.00 | 61.23 | O |
| ATOM | 2214 | N | THR | A | 320 | 30.488 | −1.936 | −11.537 | 1.00 | 61.63 | N |
| ATOM | 2215 | CA | THR | A | 320 | 31.497 | −1.880 | −10.470 | 1.00 | 62.20 | C |
| ATOM | 2216 | CB | THR | A | 320 | 32.620 | −0.850 | −10.777 | 1.00 | 62.25 | C |
| ATOM | 2217 | OG1 | THR | A | 320 | 32.176 | 0.476 | −10.455 | 1.00 | 62.39 | O |
| ATOM | 2218 | CG2 | THR | A | 320 | 33.033 | −0.914 | −12.247 | 1.00 | 62.52 | C |
| ATOM | 2219 | C | THR | A | 320 | 30.946 | −1.611 | −9.065 | 1.00 | 62.49 | C |
| ATOM | 2220 | O | THR | A | 320 | 30.968 | −2.492 | −8.200 | 1.00 | 62.58 | O |
| ATOM | 2221 | N | VAL | A | 321 | 30.451 | −0.398 | −8.840 | 1.00 | 62.79 | N |
| ATOM | 2222 | CA | VAL | A | 321 | 30.129 | 0.029 | −7.482 | 1.00 | 63.06 | C |
| ATOM | 2223 | CB | VAL | A | 321 | 30.852 | 1.360 | −7.122 | 1.00 | 63.09 | C |
| ATOM | 2224 | CG1 | VAL | A | 321 | 30.552 | 2.457 | −8.196 | 1.00 | 63.09 | C |
| ATOM | 2225 | CG2 | VAL | A | 321 | 30.489 | 1.830 | −5.677 | 1.00 | 63.21 | C |
| ATOM | 2226 | C | VAL | A | 321 | 28.625 | 0.097 | −7.157 | 1.00 | 63.21 | C |
| ATOM | 2227 | O | VAL | A | 321 | 28.177 | −0.527 | −6.190 | 1.00 | 63.23 | O |
| ATOM | 2228 | N | PHE | A | 322 | 27.846 | 0.829 | −7.953 | 1.00 | 63.40 | N |
| ATOM | 2229 | CA | PHE | A | 322 | 26.411 | 0.966 | −7.661 | 1.00 | 63.56 | C |
| ATOM | 2230 | CB | PHE | A | 322 | 26.053 | 2.392 | −7.207 | 1.00 | 63.65 | C |
| ATOM | 2231 | CG | PHE | A | 322 | 26.791 | 3.475 | −7.940 | 1.00 | 63.76 | C |
| ATOM | 2232 | CD1 | PHE | A | 322 | 27.629 | 4.341 | −7.253 | 1.00 | 63.84 | C |
| ATOM | 2233 | CE1 | PHE | A | 322 | 28.311 | 5.344 | −7.919 | 1.00 | 64.14 | C |
| ATOM | 2234 | CZ | PHE | A | 322 | 28.157 | 5.492 | −9.289 | 1.00 | 64.13 | C |
| ATOM | 2235 | CE2 | PHE | A | 322 | 27.322 | 4.636 | −9.985 | 1.00 | 64.19 | C |
| ATOM | 2236 | CD2 | PHE | A | 322 | 26.643 | 3.633 | −9.311 | 1.00 | 64.04 | C |
| ATOM | 2237 | C | PHE | A | 322 | 25.459 | 0.500 | −8.764 | 1.00 | 63.57 | C |
| ATOM | 2238 | O | PHE | A | 322 | 25.789 | 0.536 | −9.951 | 1.00 | 63.52 | O |
| ATOM | 2239 | N | GLY | A | 323 | 24.271 | 0.074 | −8.341 | 1.00 | 63.63 | N |
| ATOM | 2240 | CA | GLY | A | 323 | 23.257 | −0.470 | −9.238 | 1.00 | 63.75 | C |
| ATOM | 2241 | C | GLY | A | 323 | 23.131 | −1.967 | −9.042 | 1.00 | 63.84 | C |
| ATOM | 2242 | O | GLY | A | 323 | 22.124 | −2.574 | −9.402 | 1.00 | 63.82 | O |
| ATOM | 2243 | N | LYS | A | 324 | 24.168 | −2.547 | −8.448 | 1.00 | 63.98 | N |
| ATOM | 2244 | CA | LYS | A | 324 | 24.303 | −3.993 | −8.249 | 1.00 | 64.15 | C |
| ATOM | 2245 | CB | LYS | A | 324 | 25.266 | −4.263 | −7.084 | 1.00 | 64.21 | C |
| ATOM | 2246 | CG | LYS | A | 324 | 25.336 | −3.122 | −6.060 | 1.00 | 64.51 | C |
| ATOM | 2247 | CD | LYS | A | 324 | 26.357 | −3.392 | −4.952 | 1.00 | 64.43 | C |
| ATOM | 2248 | CE | LYS | A | 324 | 27.696 | −3.883 | −5.508 | 1.00 | 64.92 | C |
| ATOM | 2249 | NZ | LYS | A | 324 | 28.280 | −2.994 | −6.560 | 1.00 | 64.78 | N |
| ATOM | 2250 | C | LYS | A | 324 | 23.012 | −4.807 | −8.070 | 1.00 | 64.09 | C |
| ATOM | 2251 | O | LYS | A | 324 | 22.825 | −5.828 | −8.735 | 1.00 | 64.04 | O |
| ATOM | 2252 | N | GLU | A | 325 | 22.132 | −4.361 | −7.177 | 1.00 | 64.10 | N |
| ATOM | 2253 | CA | GLU | A | 325 | 20.961 | −5.152 | −6.781 | 1.00 | 64.12 | C |
| ATOM | 2254 | CB | GLU | A | 325 | 20.169 | −4.416 | −5.692 | 1.00 | 64.09 | C |
| ATOM | 2255 | CG | GLU | A | 325 | 20.990 | −4.058 | −4.459 | 1.00 | 63.96 | C |
| ATOM | 2256 | CD | GLU | A | 325 | 20.031 | −4.963 | −3.893 | 0.00 | 50.00 | C |
| ATOM | 2257 | OE1 | GLU | A | 325 | 20.090 | −5.825 | −4.783 | 0.00 | 50.00 | O |
| ATOM | 2258 | OE2 | GLU | A | 325 | 19.664 | −5.171 | −2.727 | 0.00 | 50.00 | O |
| ATOM | 2259 | C | GLU | A | 325 | 20.030 | −5.549 | −7.941 | 1.00 | 64.19 | C |
| ATOM | 2260 | O | GLU | A | 325 | 19.810 | −6.781 | −8.214 | 1.00 | 64.25 | O |
| ATOM | 2261 | N | ASN | A | 326 | 19.472 | −4.524 | −8.613 | 1.00 | 64.26 | N |
| ATOM | 2262 | CA | ASN | A | 326 | 18.505 | −4.812 | −9.679 | 1.00 | 64.33 | C |
| ATOM | 2263 | CB | ASN | A | 326 | 17.540 | −3.633 | −9.941 | 1.00 | 64.30 | C |
| ATOM | 2264 | CG | ASN | A | 326 | 18.252 | −2.335 | −10.279 | 1.00 | 64.30 | C |
| ATOM | 2265 | OD1 | ASN | A | 326 | 18.063 | −1.317 | −9.606 | 1.00 | 64.17 | O |
| ATOM | 2266 | ND2 | ASN | A | 326 | 19.058 | −2.357 | −11.338 | 1.00 | 64.50 | N |
| ATOM | 2267 | C | ASN | A | 326 | 19.181 | −5.330 | −10.958 | 1.00 | 64.35 | C |
| ATOM | 2268 | O | ASN | A | 326 | 18.546 | −5.991 | −11.784 | 1.00 | 64.40 | O |
| ATOM | 2269 | N | LYS | A | 327 | 20.472 | −5.038 | −11.104 | 1.00 | 64.30 | N |
| ATOM | 2270 | CA | LYS | A | 327 | 21.244 | −5.569 | −12.219 | 1.00 | 64.23 | C |
| ATOM | 2271 | CB | LYS | A | 327 | 22.638 | −4.943 | −12.281 | 1.00 | 64.22 | C |
| ATOM | 2272 | CG | LYS | A | 327 | 23.130 | −4.643 | −13.699 | 1.00 | 64.25 | C |
| ATOM | 2273 | CD | LYS | A | 327 | 22.954 | −5.831 | −14.639 | 1.00 | 64.27 | C |
| ATOM | 2274 | CE | LYS | A | 327 | 23.113 | −5.426 | −16.099 | 1.00 | 64.19 | C |
| ATOM | 2275 | NZ | LYS | A | 327 | 24.510 | −5.015 | −16.421 | 1.00 | 64.30 | N |
| ATOM | 2276 | C | LYS | A | 327 | 21.355 | −7.072 | −12.042 | 1.00 | 64.18 | C |
| ATOM | 2277 | O | LYS | A | 327 | 21.258 | −7.825 | −13.004 | 1.00 | 64.23 | O |
| ATOM | 2278 | N | LYS | A | 328 | 21.551 | −7.503 | −10.801 | 1.00 | 64.17 | N |
| ATOM | 2279 | CA | LYS | A | 328 | 21.573 | −8.924 | −10.490 | 1.00 | 64.23 | C |
| ATOM | 2280 | CB | LYS | A | 328 | 22.005 | −9.156 | −9.040 | 1.00 | 64.21 | C |
| ATOM | 2281 | CG | LYS | A | 328 | 21.992 | −10.616 | −8.620 | 1.00 | 64.08 | C |
| ATOM | 2282 | CD | LYS | A | 328 | 22.061 | −10.764 | −7.110 | 1.00 | 64.01 | C |
| ATOM | 2283 | CE | LYS | A | 328 | 21.635 | −12.160 | −6.682 | 1.00 | 63.97 | C |
| ATOM | 2284 | NZ | LYS | A | 328 | 21.527 | −12.286 | −5.204 | 1.00 | 63.96 | N |
| ATOM | 2285 | C | LYS | A | 328 | 20.197 | −9.536 | −10.744 | 1.00 | 64.29 | C |
| ATOM | 2286 | O | LYS | A | 328 | 20.069 | −10.507 | −11.517 | 1.00 | 64.33 | O |
| ATOM | 2287 | N | LYS | A | 329 | 19.165 | −8.960 | −10.111 | 1.00 | 64.32 | N |
| ATOM | 2288 | CA | LYS | A | 329 | 17.813 | −9.511 | −10.273 | 1.00 | 64.39 | C |
| ATOM | 2289 | CB | LYS | A | 329 | 16.788 | −8.659 | −9.523 | 1.00 | 64.37 | C |
| ATOM | 2290 | CG | LYS | A | 329 | 16.688 | −10.172 | −7.803 | 0.00 | 50.00 | C |
| ATOM | 2291 | CD | LYS | A | 329 | 15.993 | −9.545 | −6.623 | 0.00 | 50.00 | C |
| ATOM | 2292 | CE | LYS | A | 329 | 15.083 | −10.565 | −5.937 | 0.00 | 50.00 | C |
| ATOM | 2293 | NZ | LYS | A | 329 | 14.752 | −10.177 | −4.545 | 0.00 | 50.00 | N |
| ATOM | 2294 | C | LYS | A | 329 | 17.434 | −9.631 | −11.757 | 1.00 | 64.44 | C |
| ATOM | 2295 | O | LYS | A | 329 | 16.935 | −10.671 | −12.218 | 1.00 | 64.52 | O |
| ATOM | 2296 | N | GLN | A | 330 | 17.689 | −8.557 | −12.498 | 1.00 | 64.44 | N |
| ATOM | 2297 | CA | GLN | A | 330 | 17.458 | −8.520 | −13.936 | 1.00 | 64.45 | C |
| ATOM | 2298 | CB | GLN | A | 330 | 17.758 | −7.118 | −14.473 | 1.00 | 64.44 | C |
| ATOM | 2299 | CG | GLN | A | 330 | 17.843 | −7.007 | −15.986 | 1.00 | 64.39 | C |
| ATOM | 2300 | CD | GLN | A | 330 | 18.221 | −5.608 | −16.440 | 1.00 | 64.40 | C |
| ATOM | 2301 | OE1 | GLN | A | 330 | 17.777 | −4.614 | −15.862 | 1.00 | 64.30 | O |
| ATOM | 2302 | NE2 | GLN | A | 330 | 19.046 | −5.524 | −17.479 | 1.00 | 64.34 | N |
| ATOM | 2303 | C | GLN | A | 330 | 18.305 | −9.565 | −14.658 | 1.00 | 64.45 | C |
| ATOM | 2304 | O | GLN | A | 330 | 17.813 | −10.287 | −15.529 | 1.00 | 64.60 | O |
| ATOM | 2305 | N | LEU | A | 331 | 19.579 | −9.647 | −14.281 | 1.00 | 64.39 | N |
| ATOM | 2306 | CA | LEU | A | 331 | 20.495 | −10.615 | −14.874 | 1.00 | 64.37 | C |
| ATOM | 2307 | CB | LEU | A | 331 | 21.902 | −10.488 | −14.284 | 1.00 | 64.32 | C |
| ATOM | 2308 | CG | LEU | A | 331 | 22.855 | −9.519 | −14.986 | 1.00 | 64.30 | C |
| ATOM | 2309 | CD1 | LEU | A | 331 | 24.178 | −9.421 | −14.242 | 1.00 | 64.35 | C |
| ATOM | 2310 | CD2 | LEU | A | 331 | 23.081 | −9.930 | −16.430 | 1.00 | 64.38 | C |
| ATOM | 2311 | C | LEU | A | 331 | 19.983 | −12.039 | −14.718 | 1.00 | 64.41 | C |
| ATOM | 2312 | O | LEU | A | 331 | 20.171 | −12.868 | −15.610 | 1.00 | 64.39 | O |
| ATOM | 2313 | N | ILE | A | 332 | 19.335 | −12.331 | −13.593 | 1.00 | 64.44 | N |
| ATOM | 2314 | CA | ILE | A | 332 | 18.744 | −13.660 | −13.438 | 1.00 | 64.56 | C |
| ATOM | 2315 | CB | ILE | A | 332 | 18.734 | −14.168 | −11.978 | 1.00 | 64.53 | C |
| ATOM | 2316 | CG1 | ILE | A | 332 | 18.252 | −13.081 | −11.022 | 1.00 | 64.67 | C |
| ATOM | 2317 | CD1 | ILE | A | 332 | 18.518 | −13.404 | −9.570 | 1.00 | 64.93 | C |
| ATOM | 2318 | CG2 | ILE | A | 332 | 20.122 | −14.638 | −11.573 | 1.00 | 64.59 | C |
| ATOM | 2319 | C | ILE | A | 332 | 17.361 | −13.779 | −14.076 | 1.00 | 64.64 | C |
| ATOM | 2320 | O | ILE | A | 332 | 16.916 | −14.885 | −14.381 | 1.00 | 64.71 | O |
| ATOM | 2321 | N | LEU | A | 333 | 16.681 | −12.653 | −14.286 | 1.00 | 64.69 | N |
| ATOM | 2322 | CA | LEU | A | 333 | 15.474 | −12.679 | −15.120 | 1.00 | 64.86 | C |
| ATOM | 2323 | CB | LEU | A | 333 | 14.733 | −11.340 | −15.070 | 1.00 | 64.84 | C |
| ATOM | 2324 | CG | LEU | A | 333 | 13.377 | −11.329 | −14.357 | 1.00 | 64.83 | C |
| ATOM | 2325 | CD1 | LEU | A | 333 | 13.505 | −11.795 | −12.912 | 1.00 | 64.93 | C |
| ATOM | 2326 | CD2 | LEU | A | 333 | 12.750 | −9.944 | −14.431 | 1.00 | 64.92 | C |
| ATOM | 2327 | C | LEU | A | 333 | 15.821 | −13.018 | −16.568 | 1.00 | 64.95 | C |
| ATOM | 2328 | O | LEU | A | 333 | 15.066 | −13.699 | −17.265 | 1.00 | 64.92 | O |
| ATOM | 2329 | N | LYS | A | 334 | 16.982 | −12.542 | −17.002 | 1.00 | 65.06 | N |
| ATOM | 2330 | CA | LYS | A | 334 | 17.410 | −12.645 | −18.390 | 1.00 | 65.32 | C |
| ATOM | 2331 | CB | LYS | A | 334 | 18.037 | −11.303 | −18.801 | 1.00 | 65.31 | C |
| ATOM | 2332 | CG | LYS | A | 334 | 18.859 | −11.279 | −20.084 | 1.00 | 65.31 | C |
| ATOM | 2333 | CD | LYS | A | 334 | 19.751 | −10.026 | −20.101 | 1.00 | 65.18 | C |
| ATOM | 2334 | CE | LYS | A | 334 | 20.825 | −10.098 | −21.178 | 1.00 | 64.74 | C |
| ATOM | 2335 | NZ | LYS | A | 334 | 21.839 | −9.010 | −21.016 | 1.00 | 64.46 | N |
| ATOM | 2336 | C | LYS | A | 334 | 18.385 | −13.813 | −18.589 | 1.00 | 65.54 | C |
| ATOM | 2337 | O | LYS | A | 334 | 19.241 | −13.778 | −19.474 | 1.00 | 65.57 | O |
| ATOM | 2338 | N | LEU | A | 335 | 18.243 | −14.855 | −17.772 | 1.00 | 65.83 | N |
| ATOM | 2339 | CA | LEU | A | 335 | 19.176 | −15.984 | −17.815 | 1.00 | 66.16 | C |
| ATOM | 2340 | CB | LEU | A | 335 | 19.092 | −16.826 | −16.537 | 1.00 | 66.09 | C |
| ATOM | 2341 | CG | LEU | A | 335 | 20.406 | −17.326 | −15.917 | 1.00 | 66.13 | C |
| ATOM | 2342 | CD1 | LEU | A | 335 | 20.204 | −18.673 | −15.232 | 1.00 | 66.05 | C |
| ATOM | 2343 | CD2 | LEU | A | 335 | 21.536 | −17.420 | −16.931 | 1.00 | 65.95 | C |
| ATOM | 2344 | C | LEU | A | 335 | 18.990 | −16.877 | −19.044 | 1.00 | 66.47 | C |
| ATOM | 2345 | O | LEU | A | 335 | 19.964 | −17.217 | −19.710 | 1.00 | 66.51 | O |
| ATOM | 2346 | N | PRO | A | 336 | 17.739 | −17.263 | −19.354 | 1.00 | 66.81 | N |
| ATOM | 2347 | CA | PRO | A | 336 | 17.544 | −18.144 | −20.503 | 1.00 | 67.08 | C |
| ATOM | 2348 | CB | PRO | A | 336 | 16.028 | −18.367 | −20.526 | 1.00 | 67.05 | C |
| ATOM | 2349 | CG | PRO | A | 336 | 15.458 | −17.221 | −19.762 | 1.00 | 66.99 | C |
| ATOM | 2350 | CD | PRO | A | 336 | 16.465 | −16.916 | −18.702 | 1.00 | 66.85 | C |
| ATOM | 2351 | C | PRO | A | 336 | 18.010 | −17.486 | −21.797 | 1.00 | 67.39 | C |
| ATOM | 2352 | O | PRO | A | 336 | 18.262 | −18.175 | −22.786 | 1.00 | 67.44 | O |
| ATOM | 2353 | N | VAL | A | 337 | 18.123 | −16.162 | −21.781 | 1.00 | 67.78 | N |
| ATOM | 2354 | CA | VAL | A | 337 | 18.655 | −15.421 | −22.917 | 1.00 | 68.20 | C |
| ATOM | 2355 | CB | VAL | A | 337 | 18.258 | −13.931 | −22.857 | 1.00 | 68.20 | C |
| ATOM | 2356 | CG1 | VAL | A | 337 | 18.443 | −13.275 | −24.221 | 1.00 | 68.26 | C |
| ATOM | 2357 | CG2 | VAL | A | 337 | 16.817 | −13.779 | −22.384 | 1.00 | 68.27 | C |
| ATOM | 2358 | C | VAL | A | 337 | 20.177 | −15.540 | −22.917 | 1.00 | 68.46 | C |
| ATOM | 2359 | O | VAL | A | 337 | 20.796 | −15.754 | −23.964 | 1.00 | 68.80 | O |
| ATOM | 2360 | N | ILE | A | 338 | 20.769 | −15.408 | −21.733 | 1.00 | 68.70 | N |
| ATOM | 2361 | CA | ILE | A | 338 | 22.210 | −15.578 | −21.561 | 1.00 | 68.88 | C |
| ATOM | 2362 | CB | ILE | A | 338 | 22.627 | −15.468 | −20.077 | 1.00 | 68.80 | C |
| ATOM | 2363 | CG1 | ILE | A | 338 | 22.408 | −14.045 | −19.557 | 1.00 | 68.70 | C |
| ATOM | 2364 | CD1 | ILE | A | 338 | 23.413 | −13.035 | −20.078 | 1.00 | 68.75 | C |
| ATOM | 2365 | CG2 | ILE | A | 338 | 24.082 | −15.865 | −19.900 | 1.00 | 68.67 | C |
| ATOM | 2366 | C | ILE | A | 338 | 22.661 | −16.929 | −22.114 | 1.00 | 69.11 | C |
| ATOM | 2367 | O | ILE | A | 338 | 23.575 | −16.995 | −22.939 | 1.00 | 69.27 | O |
| ATOM | 2368 | N | PHE | A | 339 | 22.007 | −17.999 | −21.664 | 1.00 | 69.30 | N |
| ATOM | 2369 | CA | PHE | A | 339 | 22.327 | −19.346 | −22.133 | 1.00 | 69.45 | C |
| ATOM | 2370 | CB | PHE | A | 339 | 21.395 | −20.390 | −21.503 | 1.00 | 69.45 | C |
| ATOM | 2371 | CG | PHE | A | 339 | 21.613 | −20.598 | −20.024 | 1.00 | 69.55 | C |
| ATOM | 2372 | CD1 | PHE | A | 339 | 22.744 | −20.104 | −19.391 | 1.00 | 69.69 | C |
| ATOM | 2373 | CE1 | PHE | A | 339 | 22.944 | −20.304 | −18.035 | 1.00 | 69.63 | C |
| ATOM | 2374 | CZ | PHE | A | 339 | 22.019 | −21.017 | −17.299 | 1.00 | 69.58 | C |
| ATOM | 2375 | CE2 | PHE | A | 339 | 20.893 | −21.524 | −17.919 | 1.00 | 69.60 | C |
| ATOM | 2376 | CD2 | PHE | A | 339 | 20.695 | −21.318 | −19.273 | 1.00 | 69.56 | C |
| ATOM | 2377 | C | PHE | A | 339 | 22.248 | −19.427 | −23.656 | 1.00 | 69.52 | C |
| ATOM | 2378 | O | PHE | A | 339 | 23.170 | −19.923 | −24.314 | 1.00 | 69.66 | O |
| ATOM | 2379 | N | ALA | A | 340 | 21.146 | −18.926 | −24.208 | 1.00 | 69.56 | N |
| ATOM | 2380 | CA | ALA | A | 340 | 20.949 | −18.908 | −25.653 | 1.00 | 69.59 | C |
| ATOM | 2381 | CB | ALA | A | 340 | 19.633 | −18.214 | −26.000 | 1.00 | 69.61 | C |
| ATOM | 2382 | C | ALA | A | 340 | 22.119 | −18.218 | −26.351 | 1.00 | 69.61 | C |
| ATOM | 2383 | O | ALA | A | 340 | 22.683 | −18.750 | −27.319 | 1.00 | 69.74 | O |
| ATOM | 2384 | N | LYS | A | 341 | 22.486 | −17.035 | −25.848 | 1.00 | 69.49 | N |
| ATOM | 2385 | CA | LYS | A | 341 | 23.574 | −16.263 | −26.446 | 1.00 | 69.65 | C |
| ATOM | 2386 | CB | LYS | A | 341 | 23.660 | −14.861 | −25.828 | 1.00 | 69.67 | C |
| ATOM | 2387 | CG | LYS | A | 341 | 22.479 | −13.952 | −26.189 | 1.00 | 69.92 | C |
| ATOM | 2388 | CD | LYS | A | 341 | 22.774 | −12.477 | −25.902 | 1.00 | 69.78 | C |
| ATOM | 2389 | CE | LYS | A | 341 | 21.518 | −11.612 | −26.045 | 1.00 | 69.77 | C |
| ATOM | 2390 | NZ | LYS | A | 341 | 20.889 | −11.703 | −27.400 | 1.00 | 69.59 | N |
| ATOM | 2391 | C | LYS | A | 341 | 24.914 | −16.993 | −26.352 | 1.00 | 69.57 | C |
| ATOM | 2392 | O | LYS | A | 341 | 25.626 | −17.142 | −27.354 | 1.00 | 69.70 | O |
| ATOM | 2393 | N | ILE | A | 342 | 25.248 | −17.463 | −25.152 | 1.00 | 69.50 | N |
| ATOM | 2394 | CA | ILE | A | 342 | 26.494 | −18.200 | −24.950 | 1.00 | 69.48 | C |
| ATOM | 2395 | CB | ILE | A | 342 | 26.709 | −18.597 | −23.478 | 1.00 | 69.39 | C |
| ATOM | 2396 | CG1 | ILE | A | 342 | 26.707 | −17.354 | −22.587 | 1.00 | 69.17 | C |
| ATOM | 2397 | CD1 | ILE | A | 342 | 27.110 | −17.626 | −21.159 | 1.00 | 68.85 | C |
| ATOM | 2398 | CG2 | ILE | A | 342 | 28.024 | −19.337 | −23.320 | 1.00 | 69.32 | C |
| ATOM | 2399 | C | ILE | A | 342 | 26.565 | −19.437 | −25.846 | 1.00 | 69.61 | C |
| ATOM | 2400 | O | ILE | A | 342 | 27.632 | −19.764 | −26.364 | 1.00 | 69.60 | O |
| ATOM | 2401 | N | GLN | A | 343 | 25.436 | −20.123 | −26.027 | 1.00 | 69.77 | N |
| ATOM | 2402 | CA | GLN | A | 343 | 25.359 | −21.194 | −27.031 | 1.00 | 69.99 | C |
| ATOM | 2403 | CB | GLN | A | 343 | 23.974 | −21.856 | −27.044 | 1.00 | 69.96 | C |
| ATOM | 2404 | CG | GLN | A | 343 | 23.772 | −22.873 | −25.937 | 1.00 | 70.15 | C |
| ATOM | 2405 | CD | GLN | A | 343 | 22.383 | −23.483 | −25.957 | 1.00 | 70.33 | C |
| ATOM | 2406 | OE1 | GLN | A | 343 | 21.783 | −23.669 | −27.044 | 1.00 | 71.09 | O |
| ATOM | 2407 | NE2 | GLN | A | 343 | 21.876 | −23.816 | −24.732 | 1.00 | 70.53 | N |
| ATOM | 2408 | C | GLN | A | 343 | 25.669 | −20.640 | −28.416 | 1.00 | 70.01 | C |
| ATOM | 2409 | O | GLN | A | 343 | 26.512 | −21.177 | −29.138 | 1.00 | 70.02 | O |
| ATOM | 2410 | N | LEU | A | 344 | 24.977 | −19.562 | −28.778 | 1.00 | 70.01 | N |
| ATOM | 2411 | CA | LEU | A | 344 | 25.165 | −18.934 | −30.082 | 1.00 | 70.13 | C |
| ATOM | 2412 | CB | LEU | A | 344 | 24.382 | −17.615 | −30.176 | 1.00 | 70.20 | C |
| ATOM | 2413 | CG | LEU | A | 344 | 22.866 | −17.683 | −30.411 | 1.00 | 70.29 | C |
| ATOM | 2414 | CD1 | LEU | A | 344 | 22.234 | −16.303 | −30.260 | 1.00 | 70.28 | C |
| ATOM | 2415 | CD2 | LEU | A | 344 | 22.543 | −18.278 | −31.778 | 1.00 | 70.32 | C |
| ATOM | 2416 | C | LEU | A | 344 | 26.640 | −18.682 | −30.384 | 1.00 | 70.13 | C |
| ATOM | 2417 | O | LEU | A | 344 | 27.154 | −19.142 | −31.405 | 1.00 | 70.23 | O |
| ATOM | 2418 | N | GLU | A | 345 | 27.323 | −17.959 | −29.496 | 1.00 | 70.00 | N |
| ATOM | 2419 | CA | GLU | A | 345 | 28.670 | −17.481 | −29.815 | 1.00 | 70.11 | C |
| ATOM | 2420 | CB | GLU | A | 345 | 28.849 | −16.036 | −29.341 | 1.00 | 70.11 | C |
| ATOM | 2421 | CG | GLU | A | 345 | 28.436 | −15.805 | −27.898 | 1.00 | 70.34 | C |
| ATOM | 2422 | CD | GLU | A | 345 | 28.662 | −14.378 | −27.452 | 1.00 | 70.34 | C |
| ATOM | 2423 | OE1 | GLU | A | 345 | 29.828 | −14.018 | −27.182 | 1.00 | 70.78 | O |
| ATOM | 2424 | OE2 | GLU | A | 345 | 27.672 | −13.618 | −27.364 | 1.00 | 70.66 | O |
| ATOM | 2425 | C | GLU | A | 345 | 29.830 | −18.345 | −29.313 | 1.00 | 70.06 | C |
| ATOM | 2426 | O | GLU | A | 345 | 30.985 | −17.922 | −29.375 | 1.00 | 70.05 | O |
| ATOM | 2427 | N | HIS | A | 346 | 29.537 | −19.545 | −28.823 | 1.00 | 70.09 | N |
| ATOM | 2428 | CA | HIS | A | 346 | 30.597 | −20.438 | −28.341 | 1.00 | 70.11 | C |
| ATOM | 2429 | CB | HIS | A | 346 | 30.676 | −20.431 | −26.811 | 1.00 | 70.05 | C |
| ATOM | 2430 | CG | HIS | A | 346 | 31.545 | −19.349 | −26.253 | 1.00 | 69.94 | C |
| ATOM | 2431 | ND1 | HIS | A | 346 | 32.919 | −19.371 | −26.358 | 1.00 | 69.85 | N |
| ATOM | 2432 | CE1 | HIS | A | 346 | 33.419 | −18.296 | −25.775 | 1.00 | 70.02 | C |
| ATOM | 2433 | NE2 | HIS | A | 346 | 32.419 | −17.581 | −25.289 | 1.00 | 69.91 | N |
| ATOM | 2434 | CD2 | HIS | A | 346 | 31.236 | −18.218 | −25.574 | 1.00 | 70.00 | C |
| ATOM | 2435 | C | HIS | A | 346 | 30.444 | −21.869 | −28.841 | 1.00 | 70.14 | C |
| ATOM | 2436 | O | HIS | A | 346 | 31.236 | −22.744 | −28.486 | 1.00 | 70.16 | O |
| ATOM | 2437 | N | HIS | A | 347 | 29.425 | −22.099 | −29.661 | 1.00 | 70.14 | N |
| ATOM | 2438 | CA | HIS | A | 347 | 29.158 | −23.422 | −30.208 | 1.00 | 70.21 | C |
| ATOM | 2439 | CB | HIS | A | 347 | 30.145 | −23.753 | −31.329 | 1.00 | 70.15 | C |
| ATOM | 2440 | CG | HIS | A | 347 | 30.172 | −22.738 | −32.427 | 1.00 | 70.25 | C |
| ATOM | 2441 | ND1 | HIS | A | 347 | 31.071 | −21.693 | −32.451 | 1.00 | 70.27 | N |
| ATOM | 2442 | CE1 | HIS | A | 347 | 30.861 | −20.961 | −33.532 | 1.00 | 70.36 | C |
| ATOM | 2443 | NE2 | HIS | A | 347 | 29.859 | −21.494 | −34.208 | 1.00 | 70.39 | N |
| ATOM | 2444 | CD2 | HIS | A | 347 | 29.409 | −22.606 | −33.538 | 1.00 | 70.34 | C |
| ATOM | 2445 | C | HIS | A | 347 | 29.219 | −24.505 | −29.138 | 1.00 | 70.23 | C |
| ATOM | 2446 | O | HIS | A | 347 | 30.208 | −25.230 | −29.032 | 1.00 | 70.29 | O |
| ATOM | 2447 | N | ILE | A | 348 | 28.165 | −24.603 | −28.336 | 1.00 | 70.25 | N |
| ATOM | 2448 | CA | ILE | A | 348 | 28.014 | −25.743 | −27.441 | 1.00 | 70.37 | C |
| ATOM | 2449 | CB | ILE | A | 348 | 28.329 | −25.401 | −25.961 | 1.00 | 70.40 | C |
| ATOM | 2450 | CG1 | ILE | A | 348 | 27.603 | −24.131 | −25.524 | 1.00 | 70.55 | C |
| ATOM | 2451 | CD1 | ILE | A | 348 | 28.426 | −22.879 | −25.700 | 1.00 | 70.97 | C |
| ATOM | 2452 | CG2 | ILE | A | 348 | 29.829 | −25.226 | −25.759 | 1.00 | 70.45 | C |
| ATOM | 2453 | C | ILE | A | 348 | 26.614 | −26.336 | −27.566 | 1.00 | 70.38 | C |
| ATOM | 2454 | O | ILE | A | 348 | 25.644 | −25.618 | −27.818 | 1.00 | 70.43 | O |
| ATOM | 2455 | N | SER | A | 349 | 26.523 | −27.653 | −27.407 | 1.00 | 70.37 | N |
| ATOM | 2456 | CA | SER | A | 349 | 25.248 | −28.352 | −27.479 | 1.00 | 70.38 | C |
| ATOM | 2457 | CB | SER | A | 349 | 25.471 | −29.867 | −27.539 | 1.00 | 70.40 | C |
| ATOM | 2458 | OG | SER | A | 349 | 24.237 | −30.566 | −27.596 | 1.00 | 70.52 | O |
| ATOM | 2459 | C | SER | A | 349 | 24.382 | −28.006 | −26.275 | 1.00 | 70.34 | C |
| ATOM | 2460 | O | SER | A | 349 | 24.880 | −27.934 | −25.149 | 1.00 | 70.27 | O |
| ATOM | 2461 | N | PRO | A | 350 | 23.078 | −27.779 | −26.511 | 1.00 | 70.29 | N |
| ATOM | 2462 | CA | PRO | A | 350 | 22.123 | −27.530 | −25.431 | 1.00 | 70.22 | C |
| ATOM | 2463 | CB | PRO | A | 350 | 20.777 | −27.495 | −26.160 | 1.00 | 70.23 | C |
| ATOM | 2464 | CG | PRO | A | 350 | 21.122 | −27.067 | −27.546 | 1.00 | 70.27 | C |
| ATOM | 2465 | CD | PRO | A | 350 | 22.439 | −27.722 | −27.837 | 1.00 | 70.21 | C |
| ATOM | 2466 | C | PRO | A | 350 | 22.130 | −28.634 | −24.364 | 1.00 | 70.13 | C |
| ATOM | 2467 | O | PRO | A | 350 | 21.642 | −28.421 | −23.253 | 1.00 | 70.22 | O |
| ATOM | 2468 | N | GLY | A | 351 | 22.689 | −29.795 | −24.695 | 1.00 | 69.88 | N |
| ATOM | 2469 | CA | GLY | A | 351 | 22.773 | −30.899 | −23.746 | 1.00 | 69.54 | C |
| ATOM | 2470 | C | GLY | A | 351 | 23.820 | −30.680 | −22.669 | 1.00 | 69.38 | C |
| ATOM | 2471 | O | GLY | A | 351 | 24.040 | −31.547 | −21.818 | 1.00 | 69.41 | O |
| ATOM | 2472 | N | ASP | A | 352 | 24.468 | −29.516 | −22.700 | 1.00 | 69.15 | N |
| ATOM | 2473 | CA | ASP | A | 352 | 25.539 | −29.207 | −21.755 | 1.00 | 68.90 | C |
| ATOM | 2474 | CB | ASP | A | 352 | 26.748 | −28.614 | −22.482 | 1.00 | 68.94 | C |
| ATOM | 2475 | CG | ASP | A | 352 | 27.470 | −29.629 | −23.340 | 1.00 | 69.10 | C |
| ATOM | 2476 | OD1 | ASP | A | 352 | 28.721 | −29.587 | −23.386 | 1.00 | 69.17 | O |
| ATOM | 2477 | OD2 | ASP | A | 352 | 26.788 | −30.470 | −23.969 | 1.00 | 69.78 | O |
| ATOM | 2478 | C | ASP | A | 352 | 25.097 | −28.251 | −20.653 | 1.00 | 68.71 | C |
| ATOM | 2479 | O | ASP | A | 352 | 25.743 | −28.157 | −19.610 | 1.00 | 68.69 | O |
| ATOM | 2480 | N | PHE | A | 353 | 24.000 | −27.541 | −20.890 | 1.00 | 68.50 | N |
| ATOM | 2481 | CA | PHE | A | 353 | 23.536 | −26.519 | −19.955 | 1.00 | 68.34 | C |
| ATOM | 2482 | CB | PHE | A | 353 | 22.700 | −25.470 | −20.683 | 1.00 | 68.32 | C |
| ATOM | 2483 | CG | PHE | A | 353 | 23.518 | −24.413 | −21.348 | 1.00 | 68.40 | C |
| ATOM | 2484 | CD1 | PHE | A | 353 | 23.773 | −23.213 | −20.704 | 1.00 | 68.54 | C |
| ATOM | 2485 | CE1 | PHE | A | 353 | 24.534 | −22.233 | −21.312 | 1.00 | 68.49 | C |
| ATOM | 2486 | CZ | PHE | A | 353 | 25.053 | −22.448 | −22.571 | 1.00 | 68.51 | C |
| ATOM | 2487 | CE2 | PHE | A | 353 | 24.811 | −23.646 | −23.220 | 1.00 | 68.68 | C |
| ATOM | 2488 | CD2 | PHE | A | 353 | 24.049 | −24.620 | −22.608 | 1.00 | 68.56 | C |
| ATOM | 2489 | C | PHE | A | 353 | 22.761 | −27.066 | −18.763 | 1.00 | 68.21 | C |
| ATOM | 2490 | O | PHE | A | 353 | 21.997 | −28.021 | −18.896 | 1.00 | 68.28 | O |
| ATOM | 2491 | N | PRO | A | 354 | 22.949 | −26.445 | −17.589 | 1.00 | 68.04 | N |
| ATOM | 2492 | CA | PRO | A | 354 | 22.224 | −26.839 | −16.385 | 1.00 | 67.82 | C |
| ATOM | 2493 | CB | PRO | A | 354 | 22.931 | −26.057 | −15.267 | 1.00 | 67.87 | C |
| ATOM | 2494 | CG | PRO | A | 354 | 24.145 | −25.426 | −15.899 | 1.00 | 67.91 | C |
| ATOM | 2495 | CD | PRO | A | 354 | 23.859 | −25.313 | −17.348 | 1.00 | 67.93 | C |
| ATOM | 2496 | C | PRO | A | 354 | 20.773 | −26.392 | −16.476 | 1.00 | 67.54 | C |
| ATOM | 2497 | O | PRO | A | 354 | 20.406 | −25.682 | −17.412 | 1.00 | 67.44 | O |
| ATOM | 2498 | N | ASP | A | 355 | 19.956 | −26.806 | −15.512 | 1.00 | 67.25 | N |
| ATOM | 2499 | CA | ASP | A | 355 | 18.591 | −26.307 | −15.419 | 1.00 | 66.95 | C |
| ATOM | 2500 | CB | ASP | A | 355 | 17.806 | −27.057 | −14.341 | 1.00 | 66.98 | C |
| ATOM | 2501 | CG | ASP | A | 355 | 16.373 | −26.569 | −14.216 | 1.00 | 67.19 | C |
| ATOM | 2502 | OD1 | ASP | A | 355 | 15.459 | −27.421 | −14.187 | 1.00 | 67.62 | O |
| ATOM | 2503 | OD2 | ASP | A | 355 | 16.155 | −25.338 | −14.148 | 1.00 | 67.10 | O |
| ATOM | 2504 | C | ASP | A | 355 | 18.627 | −24.813 | −15.111 | 1.00 | 66.67 | C |
| ATOM | 2505 | O | ASP | A | 355 | 19.260 | −24.383 | −14.144 | 1.00 | 66.78 | O |
| ATOM | 2506 | N | CYS | A | 356 | 17.945 | −24.029 | −15.940 | 1.00 | 66.16 | N |
| ATOM | 2507 | CA | CYS | A | 356 | 17.977 | −22.576 | −15.834 | 1.00 | 65.72 | C |
| ATOM | 2508 | CB | CYS | A | 356 | 17.156 | −21.950 | −16.964 | 1.00 | 65.76 | C |
| ATOM | 2509 | SG | CYS | A | 356 | 17.529 | −20.216 | −17.290 | 1.00 | 65.98 | S |
| ATOM | 2510 | C | CYS | A | 356 | 17.470 | −22.090 | −14.478 | 1.00 | 65.43 | C |
| ATOM | 2511 | O | CYS | A | 356 | 18.068 | −21.203 | −13.864 | 1.00 | 64.92 | O |
| ATOM | 2512 | N | GLN | A | 357 | 16.378 | −22.689 | −14.010 | 1.00 | 65.25 | N |
| ATOM | 2513 | CA | GLN | A | 357 | 15.719 | −22.247 | −12.780 | 1.00 | 65.06 | C |
| ATOM | 2514 | CB | GLN | A | 357 | 14.262 | −22.723 | −12.746 | 1.00 | 65.11 | C |
| ATOM | 2515 | CG | GLN | A | 357 | 13.336 | −21.874 | −13.615 | 1.00 | 65.39 | C |
| ATOM | 2516 | CD | GLN | A | 357 | 12.050 | −22.588 | −13.988 | 1.00 | 65.96 | C |
| ATOM | 2517 | OE1 | GLN | A | 357 | 11.004 | −21.939 | −14.178 | 1.00 | 66.00 | O |
| ATOM | 2518 | NE2 | GLN | A | 357 | 12.123 | −23.933 | −14.100 | 1.00 | 66.01 | N |
| ATOM | 2519 | C | GLN | A | 357 | 16.466 | −22.615 | −11.492 | 1.00 | 64.82 | C |
| ATOM | 2520 | O | GLN | A | 357 | 16.503 | −21.825 | −10.541 | 1.00 | 64.50 | O |
| ATOM | 2521 | N | LYS | A | 358 | 17.061 | −23.805 | −11.458 | 1.00 | 64.56 | N |
| ATOM | 2522 | CA | LYS | A | 358 | 17.898 | −24.192 | −10.327 | 1.00 | 64.41 | C |
| ATOM | 2523 | CB | LYS | A | 358 | 18.410 | −25.623 | −10.501 | 1.00 | 64.55 | C |
| ATOM | 2524 | CG | LYS | A | 358 | 19.047 | −26.218 | −9.249 | 1.00 | 65.05 | C |
| ATOM | 2525 | CD | LYS | A | 358 | 18.000 | −26.546 | −8.187 | 1.00 | 65.74 | C |
| ATOM | 2526 | CE | LYS | A | 358 | 18.640 | −27.141 | −6.936 | 1.00 | 66.04 | C |
| ATOM | 2527 | NZ | LYS | A | 358 | 17.621 | −27.613 | −5.948 | 1.00 | 66.17 | N |
| ATOM | 2528 | C | LYS | A | 358 | 19.067 | −23.215 | −10.243 | 1.00 | 64.12 | C |
| ATOM | 2529 | O | LYS | A | 358 | 19.329 | −22.600 | −9.193 | 1.00 | 63.78 | O |
| ATOM | 2530 | N | MET | A | 359 | 19.752 | −23.074 | −11.374 | 1.00 | 63.91 | N |
| ATOM | 2531 | CA | MET | A | 359 | 20.818 | −22.101 | −11.531 | 1.00 | 63.82 | C |
| ATOM | 2532 | CB | MET | A | 359 | 21.298 | −22.083 | −12.984 | 1.00 | 63.91 | C |
| ATOM | 2533 | CG | MET | A | 359 | 22.634 | −21.389 | −13.214 | 1.00 | 64.20 | C |
| ATOM | 2534 | SD | MET | A | 359 | 24.047 | −22.269 | −12.508 | 1.00 | 65.02 | S |
| ATOM | 2535 | CE | MET | A | 359 | 25.409 | −21.348 | −13.220 | 1.00 | 64.30 | C |
| ATOM | 2536 | C | MET | A | 359 | 20.322 | −20.719 | −11.117 | 1.00 | 63.69 | C |
| ATOM | 2537 | O | MET | A | 359 | 21.086 | −19.907 | −10.602 | 1.00 | 63.41 | O |
| ATOM | 2538 | N | GLN | A | 360 | 19.037 | −20.459 | −11.328 | 1.00 | 63.78 | N |
| ATOM | 2539 | CA | GLN | A | 360 | 18.454 | −19.186 | −10.916 | 1.00 | 63.51 | C |
| ATOM | 2540 | CB | GLN | A | 360 | 17.126 | −18.937 | −11.632 | 1.00 | 63.59 | C |
| ATOM | 2541 | CG | GLN | A | 360 | 17.299 | −18.365 | −13.029 | 1.00 | 63.62 | C |
| ATOM | 2542 | CD | GLN | A | 360 | 16.031 | −18.412 | −13.856 | 1.00 | 63.50 | C |
| ATOM | 2543 | OE1 | GLN | A | 360 | 15.815 | −17.566 | −14.722 | 1.00 | 63.52 | O |
| ATOM | 2544 | NE2 | GLN | A | 360 | 15.187 | −19.403 | −13.595 | 1.00 | 63.46 | N |
| ATOM | 2545 | C | GLN | A | 360 | 18.297 | −19.059 | −9.399 | 1.00 | 63.40 | C |
| ATOM | 2546 | O | GLN | A | 360 | 18.546 | −17.991 | −8.834 | 1.00 | 63.16 | O |
| ATOM | 2547 | N | GLU | A | 361 | 17.895 | −20.144 | −8.741 | 1.00 | 63.45 | N |
| ATOM | 2548 | CA | GLU | A | 361 | 17.802 | −20.151 | −7.279 | 1.00 | 63.14 | C |
| ATOM | 2549 | CB | GLU | A | 361 | 17.142 | −21.442 | −6.790 | 1.00 | 63.14 | C |
| ATOM | 2550 | CG | GLU | A | 361 | 15.901 | −22.350 | −6.363 | 0.00 | 50.00 | C |
| ATOM | 2551 | CD | GLU | A | 361 | 15.696 | −23.530 | −5.435 | 0.00 | 50.00 | C |
| ATOM | 2552 | OE1 | GLU | A | 361 | 16.150 | −24.642 | −5.776 | 0.00 | 50.00 | O |
| ATOM | 2553 | OE2 | GLU | A | 361 | 15.081 | −23.346 | −4.363 | 0.00 | 50.00 | O |
| ATOM | 2554 | C | GLU | A | 361 | 19.184 | −19.978 | −6.637 | 1.00 | 63.01 | C |
| ATOM | 2555 | O | GLU | A | 361 | 19.382 | −19.152 | −5.715 | 1.00 | 62.82 | O |
| ATOM | 2556 | N | LEU | A | 362 | 20.148 | −20.751 | −7.138 | 1.00 | 62.93 | N |
| ATOM | 2557 | CA | LEU | A | 362 | 21.511 | −20.662 | −6.617 | 1.00 | 62.55 | C |
| ATOM | 2558 | CB | LEU | A | 362 | 22.361 | −21.809 | −7.164 | 1.00 | 62.51 | C |
| ATOM | 2559 | CG | LEU | A | 362 | 21.784 | −23.205 | −6.916 | 1.00 | 62.22 | C |
| ATOM | 2560 | CD1 | LEU | A | 362 | 22.741 | −24.272 | −7.416 | 1.00 | 61.94 | C |
| ATOM | 2561 | CD2 | LEU | A | 362 | 21.471 | −23.409 | −5.440 | 1.00 | 61.87 | C |
| ATOM | 2562 | C | LEU | A | 362 | 22.154 | −19.299 | −6.921 | 1.00 | 62.44 | C |
| ATOM | 2563 | O | LEU | A | 362 | 22.811 | −18.678 | −6.052 | 1.00 | 62.11 | O |
| ATOM | 2564 | N | LEU | A | 363 | 21.960 | −18.832 | −8.156 | 1.00 | 62.56 | N |
| ATOM | 2565 | CA | LEU | A | 363 | 22.412 | −17.501 | −8.530 | 1.00 | 62.37 | C |
| ATOM | 2566 | CB | LEU | A | 363 | 22.080 | −17.199 | −9.993 | 1.00 | 62.32 | C |
| ATOM | 2567 | CG | LEU | A | 363 | 23.212 | −17.346 | −11.013 | 1.00 | 62.11 | C |
| ATOM | 2568 | CD1 | LEU | A | 363 | 24.325 | −16.381 | −10.684 | 1.00 | 62.15 | C |
| ATOM | 2569 | CD2 | LEU | A | 363 | 23.753 | −18.759 | −11.056 | 1.00 | 61.97 | C |
| ATOM | 2570 | C | LEU | A | 363 | 21.759 | −16.483 | −7.609 | 1.00 | 62.38 | C |
| ATOM | 2571 | O | LEU | A | 363 | 22.362 | −15.468 | −7.261 | 1.00 | 62.29 | O |
| ATOM | 2572 | N | MET | A | 364 | 20.521 | −16.770 | −7.213 | 1.00 | 62.56 | N |
| ATOM | 2573 | CA | MET | A | 364 | 19.821 | −15.949 | −6.229 | 1.00 | 62.46 | C |
| ATOM | 2574 | CB | MET | A | 364 | 18.376 | −16.436 | −6.047 | 1.00 | 62.50 | C |
| ATOM | 2575 | CG | MET | A | 364 | 17.449 | −15.444 | −5.357 | 1.00 | 62.64 | C |
| ATOM | 2576 | SD | MET | A | 364 | 17.255 | −13.900 | −6.279 | 1.00 | 63.45 | S |
| ATOM | 2577 | CE | MET | A | 364 | 15.974 | −13.066 | −5.331 | 1.00 | 63.02 | C |
| ATOM | 2578 | C | MET | A | 364 | 20.576 | −16.000 | −4.903 | 1.00 | 62.32 | C |
| ATOM | 2579 | O | MET | A | 364 | 20.579 | −15.030 | −4.142 | 1.00 | 62.24 | O |
| ATOM | 2580 | N | ALA | A | 365 | 21.227 | −17.132 | −4.638 | 1.00 | 62.25 | N |
| ATOM | 2581 | CA | ALA | A | 365 | 22.014 | −17.277 | −3.401 | 1.00 | 61.89 | C |
| ATOM | 2582 | CB | ALA | A | 365 | 22.130 | −18.749 | −3.012 | 1.00 | 61.88 | C |
| ATOM | 2583 | C | ALA | A | 365 | 23.406 | −16.615 | −3.399 | 1.00 | 61.73 | C |
| ATOM | 2584 | O | ALA | A | 365 | 23.901 | −16.219 | −2.344 | 1.00 | 61.65 | O |
| ATOM | 2585 | N | HIS | A | 366 | 24.039 | −16.491 | −4.565 | 1.00 | 61.58 | N |
| ATOM | 2586 | CA | HIS | A | 366 | 25.428 | −15.979 | −4.623 | 1.00 | 61.07 | C |
| ATOM | 2587 | CB | HIS | A | 366 | 26.178 | −16.621 | −5.786 | 1.00 | 61.07 | C |
| ATOM | 2588 | CG | HIS | A | 366 | 26.701 | −17.983 | −5.478 | 1.00 | 60.77 | C |
| ATOM | 2589 | ND1 | HIS | A | 366 | 25.881 | −19.026 | −5.107 | 1.00 | 60.88 | N |
| ATOM | 2590 | CE1 | HIS | A | 366 | 26.613 | −20.104 | −4.894 | 1.00 | 61.03 | C |
| ATOM | 2591 | NE2 | HIS | A | 366 | 27.879 | −19.796 | −5.114 | 1.00 | 61.08 | N |
| ATOM | 2592 | CD2 | HIS | A | 366 | 27.961 | −18.475 | −5.481 | 1.00 | 60.66 | C |
| ATOM | 2593 | C | HIS | A | 366 | 25.606 | −14.462 | −4.714 | 1.00 | 60.91 | C |
| ATOM | 2594 | O | HIS | A | 366 | 24.659 | −13.732 | −4.999 | 1.00 | 60.95 | O |
| ATOM | 2595 | N | ASP | A | 367 | 26.837 | −14.001 | −4.476 | 1.00 | 60.67 | N |
| ATOM | 2596 | CA | ASP | A | 367 | 27.209 | −12.600 | −4.702 | 1.00 | 60.54 | C |
| ATOM | 2597 | CB | ASP | A | 367 | 28.089 | −12.052 | −3.576 | 1.00 | 60.59 | C |
| ATOM | 2598 | CG | ASP | A | 367 | 28.551 | −10.615 | −3.839 | 1.00 | 60.69 | C |
| ATOM | 2599 | OD1 | ASP | A | 367 | 29.141 | −9.993 | −2.927 | 1.00 | 60.49 | O |
| ATOM | 2600 | OD2 | ASP | A | 367 | 28.324 | −10.103 | −4.958 | 1.00 | 60.90 | O |
| ATOM | 2601 | C | ASP | A | 367 | 27.947 | −12.442 | −6.020 | 1.00 | 60.51 | C |
| ATOM | 2602 | O | ASP | A | 367 | 28.946 | −13.117 | −6.273 | 1.00 | 60.68 | O |
| ATOM | 2603 | N | PHE | A | 368 | 27.468 | −11.519 | −6.845 | 1.00 | 60.30 | N |
| ATOM | 2604 | CA | PHE | A | 368 | 27.980 | −11.376 | −8.194 | 1.00 | 60.01 | C |
| ATOM | 2605 | CB | PHE | A | 368 | 26.956 | −10.666 | −9.076 | 1.00 | 60.10 | C |
| ATOM | 2606 | CG | PHE | A | 368 | 25.846 | −11.562 | −9.547 | 1.00 | 60.32 | C |
| ATOM | 2607 | CD1 | PHE | A | 368 | 25.315 | −12.532 | −8.707 | 1.00 | 60.54 | C |
| ATOM | 2608 | CE1 | PHE | A | 368 | 24.295 | −13.358 | −9.133 | 1.00 | 60.45 | C |
| ATOM | 2609 | CZ | PHE | A | 368 | 23.784 | −13.217 | −10.408 | 1.00 | 60.53 | C |
| ATOM | 2610 | CE2 | PHE | A | 368 | 24.299 | −12.251 | −11.256 | 1.00 | 60.66 | C |
| ATOM | 2611 | CD2 | PHE | A | 368 | 25.323 | −11.430 | −10.823 | 1.00 | 60.62 | C |
| ATOM | 2612 | C | PHE | A | 368 | 29.324 | −10.673 | −8.238 | 1.00 | 59.80 | C |
| ATOM | 2613 | O | PHE | A | 368 | 30.204 | −11.067 | −8.998 | 1.00 | 59.86 | O |
| ATOM | 2614 | N | THR | A | 369 | 29.486 | −9.641 | −7.417 | 1.00 | 59.53 | N |
| ATOM | 2615 | CA | THR | A | 369 | 30.717 | −8.854 | −7.432 | 1.00 | 59.25 | C |
| ATOM | 2616 | CB | THR | A | 369 | 30.613 | −7.593 | −6.543 | 1.00 | 59.18 | C |
| ATOM | 2617 | OG1 | THR | A | 369 | 30.494 | −7.974 | −5.167 | 1.00 | 59.25 | O |
| ATOM | 2618 | CG2 | THR | A | 369 | 29.407 | −6.761 | −6.943 | 1.00 | 59.10 | C |
| ATOM | 2619 | C | THR | A | 369 | 31.927 | −9.691 | −7.025 | 1.00 | 59.10 | C |
| ATOM | 2620 | O | THR | A | 369 | 33.068 | −9.311 | −7.280 | 1.00 | 58.97 | O |
| ATOM | 2621 | N | LYS | A | 370 | 31.670 | −10.840 | −6.407 | 1.00 | 59.01 | N |
| ATOM | 2622 | CA | LYS | A | 370 | 32.740 | −11.725 | −5.960 | 1.00 | 58.97 | C |
| ATOM | 2623 | CB | LYS | A | 370 | 32.318 | −12.482 | −4.699 | 1.00 | 59.01 | C |
| ATOM | 2624 | CG | LYS | A | 370 | 31.859 | −11.596 | −3.550 | 1.00 | 59.07 | C |
| ATOM | 2625 | CD | LYS | A | 370 | 33.008 | −11.175 | −2.649 | 1.00 | 59.12 | C |
| ATOM | 2626 | CE | LYS | A | 370 | 32.490 | −10.481 | −1.392 | 1.00 | 59.61 | C |
| ATOM | 2627 | NZ | LYS | A | 370 | 31.488 | −11.309 | −0.638 | 1.00 | 59.98 | N |
| ATOM | 2628 | C | LYS | A | 370 | 33.152 | −12.715 | −7.047 | 1.00 | 58.95 | C |
| ATOM | 2629 | O | LYS | A | 370 | 34.235 | −13.292 | −6.982 | 1.00 | 58.95 | O |
| ATOM | 2630 | N | PHE | A | 371 | 32.285 | −12.914 | −8.039 | 1.00 | 58.98 | N |
| ATOM | 2631 | CA | PHE | A | 371 | 32.595 | −13.790 | −9.170 | 1.00 | 59.03 | C |
| ATOM | 2632 | CB | PHE | A | 371 | 31.474 | −13.759 | −10.214 | 1.00 | 58.86 | C |
| ATOM | 2633 | CG | PHE | A | 371 | 30.216 | −14.459 | −9.785 | 1.00 | 58.79 | C |
| ATOM | 2634 | CD1 | PHE | A | 371 | 28.979 | −14.003 | −10.210 | 1.00 | 58.52 | C |
| ATOM | 2635 | CE1 | PHE | A | 371 | 27.818 | −14.644 | −9.823 | 1.00 | 58.30 | C |
| ATOM | 2636 | CZ | PHE | A | 371 | 27.883 | −15.752 | −9.000 | 1.00 | 58.32 | C |
| ATOM | 2637 | CE2 | PHE | A | 371 | 29.106 | −16.218 | −8.569 | 1.00 | 58.37 | C |
| ATOM | 2638 | CD2 | PHE | A | 371 | 30.266 | −15.574 | −8.961 | 1.00 | 58.75 | C |
| ATOM | 2639 | C | PHE | A | 371 | 33.902 | −13.386 | −9.840 | 1.00 | 59.21 | C |
| ATOM | 2640 | O | PHE | A | 371 | 34.291 | −12.223 | −9.804 | 1.00 | 59.25 | O |
| ATOM | 2641 | N | HIS | A | 372 | 34.579 | −14.346 | −10.456 | 1.00 | 59.50 | N |
| ATOM | 2642 | CA | HIS | A | 372 | 35.789 | −14.040 | −11.206 | 1.00 | 59.80 | C |
| ATOM | 2643 | CB | HIS | A | 372 | 36.798 | −15.180 | −11.093 | 1.00 | 59.85 | C |
| ATOM | 2644 | CG | HIS | A | 372 | 37.463 | −15.261 | −9.756 | 1.00 | 59.95 | C |
| ATOM | 2645 | ND1 | HIS | A | 372 | 38.399 | −14.342 | −9.335 | 1.00 | 59.92 | N |
| ATOM | 2646 | CE1 | HIS | A | 372 | 38.814 | −14.663 | −8.123 | 1.00 | 60.28 | C |
| ATOM | 2647 | NE2 | HIS | A | 372 | 38.178 | −15.756 | −7.741 | 1.00 | 60.47 | N |
| ATOM | 2648 | CD2 | HIS | A | 372 | 37.327 | −16.150 | −8.744 | 1.00 | 60.21 | C |
| ATOM | 2649 | C | HIS | A | 372 | 35.468 | −13.754 | −12.668 | 1.00 | 59.98 | C |
| ATOM | 2650 | O | HIS | A | 372 | 34.385 | −14.092 | −13.149 | 1.00 | 60.08 | O |
| ATOM | 2651 | N | SER | A | 373 | 36.413 | −13.130 | −13.370 | 1.00 | 60.16 | N |
| ATOM | 2652 | CA | SER | A | 373 | 36.231 | −12.788 | −14.783 | 1.00 | 60.24 | C |
| ATOM | 2653 | CB | SER | A | 373 | 37.012 | −11.521 | −15.135 | 1.00 | 60.28 | C |
| ATOM | 2654 | OG | SER | A | 373 | 36.688 | −10.461 | −14.252 | 1.00 | 60.74 | O |
| ATOM | 2655 | C | SER | A | 373 | 36.664 | −13.918 | −15.705 | 1.00 | 60.17 | C |
| ATOM | 2656 | O | SER | A | 373 | 37.354 | −14.847 | −15.291 | 1.00 | 60.00 | O |
| ATOM | 2657 | N | LEU | A | 374 | 36.259 | −13.828 | −16.964 | 1.00 | 60.35 | N |
| ATOM | 2658 | CA | LEU | A | 374 | 36.661 | −14.813 | −17.955 | 1.00 | 60.62 | C |
| ATOM | 2659 | CB | LEU | A | 374 | 36.125 | −14.424 | −19.333 | 1.00 | 60.56 | C |
| ATOM | 2660 | CG | LEU | A | 374 | 36.072 | −15.531 | −20.384 | 1.00 | 60.72 | C |
| ATOM | 2661 | CD1 | LEU | A | 374 | 34.981 | −15.247 | −21.409 | 1.00 | 61.21 | C |
| ATOM | 2662 | CD2 | LEU | A | 374 | 37.420 | −15.707 | −21.060 | 1.00 | 60.74 | C |
| ATOM | 2663 | C | LEU | A | 374 | 38.184 | −14.936 | −17.972 | 1.00 | 60.76 | C |
| ATOM | 2664 | O | LEU | A | 374 | 38.894 | −13.933 | −17.917 | 1.00 | 60.93 | O |
| ATOM | 2665 | N | LYS | A | 375 | 38.682 | −16.167 | −18.030 | 1.00 | 60.82 | N |
| ATOM | 2666 | CA | LYS | A | 375 | 40.120 | −16.420 | −18.015 | 1.00 | 60.84 | C |
| ATOM | 2667 | CB | LYS | A | 375 | 40.535 | −17.002 | −16.664 | 1.00 | 60.91 | C |
| ATOM | 2668 | CG | LYS | A | 375 | 40.369 | −16.044 | −15.498 | 1.00 | 61.48 | C |
| ATOM | 2669 | CD | LYS | A | 375 | 41.697 | −15.434 | −15.084 | 1.00 | 62.71 | C |
| ATOM | 2670 | CE | LYS | A | 375 | 42.553 | −16.457 | −14.337 | 1.00 | 63.51 | C |
| ATOM | 2671 | NZ | LYS | A | 375 | 43.854 | −15.899 | −13.868 | 1.00 | 63.82 | N |
| ATOM | 2672 | C | LYS | A | 375 | 40.491 | −17.381 | −19.138 | 1.00 | 60.68 | C |
| ATOM | 2673 | O | LYS | A | 375 | 40.479 | −18.595 | −18.946 | 1.00 | 60.73 | O |
| ATOM | 2674 | N | PRO | A | 376 | 40.838 | −16.837 | −20.313 | 1.00 | 60.57 | N |
| ATOM | 2675 | CA | PRO | A | 376 | 41.018 | −17.632 | −21.526 | 1.00 | 60.57 | C |
| ATOM | 2676 | CB | PRO | A | 376 | 41.521 | −16.607 | −22.545 | 1.00 | 60.60 | C |
| ATOM | 2677 | CG | PRO | A | 376 | 41.046 | −15.295 | −22.034 | 1.00 | 60.61 | C |
| ATOM | 2678 | CD | PRO | A | 376 | 41.097 | −15.407 | −20.544 | 1.00 | 60.55 | C |
| ATOM | 2679 | C | PRO | A | 376 | 42.042 | −18.746 | −21.353 | 1.00 | 60.59 | C |
| ATOM | 2680 | O | PRO | A | 376 | 41.907 | −19.809 | −21.956 | 1.00 | 60.69 | O |
| ATOM | 2681 | N | LYS | A | 377 | 43.050 | −18.495 | −20.525 | 1.00 | 60.59 | N |
| ATOM | 2682 | CA | LYS | A | 377 | 44.131 | −19.446 | −20.285 | 1.00 | 60.71 | C |
| ATOM | 2683 | CB | LYS | A | 377 | 45.129 | −18.839 | −19.295 | 1.00 | 60.74 | C |
| ATOM | 2684 | CG | LYS | A | 377 | 44.517 | −17.771 | −18.384 | 1.00 | 61.05 | C |
| ATOM | 2685 | CD | LYS | A | 377 | 45.457 | −17.378 | −17.246 | 1.00 | 61.19 | C |
| ATOM | 2686 | CE | LYS | A | 377 | 45.573 | −18.489 | −16.201 | 1.00 | 61.82 | C |
| ATOM | 2687 | NZ | LYS | A | 377 | 46.528 | −18.146 | −15.102 | 1.00 | 62.06 | N |
| ATOM | 2688 | C | LYS | A | 377 | 43.654 | −20.821 | −19.798 | 1.00 | 60.59 | C |
| ATOM | 2689 | O | LYS | A | 377 | 43.932 | −21.849 | −20.429 | 1.00 | 60.69 | O |
| ATOM | 2690 | N | LEU | A | 378 | 42.935 | −20.834 | −18.678 | 1.00 | 60.51 | N |
| ATOM | 2691 | CA | LEU | A | 378 | 42.450 | −22.076 | −18.077 | 1.00 | 60.41 | C |
| ATOM | 2692 | CB | LEU | A | 378 | 41.743 | −21.783 | −16.757 | 1.00 | 60.35 | C |
| ATOM | 2693 | CG | LEU | A | 378 | 42.521 | −20.939 | −15.743 | 1.00 | 60.12 | C |
| ATOM | 2694 | CD1 | LEU | A | 378 | 41.617 | −20.552 | −14.588 | 1.00 | 59.86 | C |
| ATOM | 2695 | CD2 | LEU | A | 378 | 43.767 | −21.663 | −15.242 | 1.00 | 59.70 | C |
| ATOM | 2696 | C | LEU | A | 378 | 41.507 | −22.807 | −19.022 | 1.00 | 60.42 | C |
| ATOM | 2697 | O | LEU | A | 378 | 41.628 | −24.019 | −19.238 | 1.00 | 60.64 | O |
| ATOM | 2698 | N | LEU | A | 379 | 40.564 | −22.056 | −19.578 | 1.00 | 60.36 | N |
| ATOM | 2699 | CA | LEU | A | 379 | 39.686 | −22.568 | −20.617 | 1.00 | 60.34 | C |
| ATOM | 2700 | CB | LEU | A | 379 | 38.868 | −21.425 | −21.214 | 1.00 | 60.18 | C |
| ATOM | 2701 | CG | LEU | A | 379 | 37.657 | −21.020 | −20.372 | 1.00 | 59.81 | C |
| ATOM | 2702 | CD1 | LEU | A | 379 | 37.524 | −19.515 | −20.250 | 1.00 | 59.39 | C |
| ATOM | 2703 | CD2 | LEU | A | 379 | 36.394 | −21.632 | −20.952 | 1.00 | 59.83 | C |
| ATOM | 2704 | C | LEU | A | 379 | 40.493 | −23.284 | −21.695 | 1.00 | 60.50 | C |
| ATOM | 2705 | O | LEU | A | 379 | 40.278 | −24.470 | −21.961 | 1.00 | 60.81 | O |
| ATOM | 2706 | N | GLU | A | 380 | 41.432 | −22.567 | −22.304 | 1.00 | 60.65 | N |
| ATOM | 2707 | CA | GLU | A | 380 | 42.308 | −23.167 | −23.303 | 1.00 | 60.83 | C |
| ATOM | 2708 | CB | GLU | A | 380 | 43.438 | −22.214 | −23.691 | 1.00 | 60.85 | C |
| ATOM | 2709 | CG | GLU | A | 380 | 43.118 | −21.304 | −24.858 | 1.00 | 60.88 | C |
| ATOM | 2710 | CD | GLU | A | 380 | 44.355 | −20.937 | −25.648 | 1.00 | 61.03 | C |
| ATOM | 2711 | OE1 | GLU | A | 380 | 44.281 | −20.931 | −26.892 | 1.00 | 61.38 | O |
| ATOM | 2712 | OE2 | GLU | A | 380 | 45.407 | −20.673 | −25.029 | 1.00 | 61.20 | O |
| ATOM | 2713 | C | GLU | A | 380 | 42.896 | −24.470 | −22.786 | 1.00 | 60.92 | C |
| ATOM | 2714 | O | GLU | A | 380 | 42.872 | −25.499 | −23.481 | 1.00 | 61.26 | O |
| ATOM | 2715 | N | ALA | A | 381 | 43.424 | −24.420 | −21.564 | 1.00 | 60.89 | N |
| ATOM | 2716 | CA | ALA | A | 381 | 43.984 | −25.606 | −20.931 | 1.00 | 61.02 | C |
| ATOM | 2717 | CB | ALA | A | 381 | 44.320 | −25.322 | −19.480 | 1.00 | 60.97 | C |
| ATOM | 2718 | C | ALA | A | 381 | 42.991 | −26.754 | −21.036 | 1.00 | 61.14 | C |
| ATOM | 2719 | O | ALA | A | 381 | 43.344 | −27.872 | −21.425 | 1.00 | 61.43 | O |
| ATOM | 2720 | N | LEU | A | 382 | 41.741 | −26.460 | −20.701 | 1.00 | 61.13 | N |
| ATOM | 2721 | CA | LEU | A | 382 | 40.666 | −27.438 | −20.800 | 1.00 | 61.34 | C |
| ATOM | 2722 | CB | LEU | A | 382 | 39.365 | −26.808 | −20.291 | 1.00 | 61.35 | C |
| ATOM | 2723 | CG | LEU | A | 382 | 38.199 | −27.671 | −19.802 | 1.00 | 61.48 | C |
| ATOM | 2724 | CD1 | LEU | A | 382 | 37.136 | −27.835 | −20.885 | 1.00 | 61.27 | C |
| ATOM | 2725 | CD2 | LEU | A | 382 | 38.694 | −29.019 | −19.281 | 1.00 | 62.11 | C |
| ATOM | 2726 | C | LEU | A | 382 | 40.506 | −27.964 | −22.236 | 1.00 | 61.46 | C |
| ATOM | 2727 | O | LEU | A | 382 | 40.736 | −29.157 | −22.509 | 1.00 | 61.78 | O |
| ATOM | 2728 | N | ASP | A | 383 | 40.127 | −27.070 | −23.150 | 1.00 | 61.44 | N |
| ATOM | 2729 | CA | ASP | A | 383 | 39.867 | −27.453 | −24.543 | 1.00 | 61.75 | C |
| ATOM | 2730 | CB | ASP | A | 383 | 39.743 | −26.209 | −25.431 | 1.00 | 61.77 | C |
| ATOM | 2731 | CG | ASP | A | 383 | 38.778 | −25.172 | −24.868 | 1.00 | 61.90 | C |
| ATOM | 2732 | OD1 | ASP | A | 383 | 38.498 | −25.208 | −23.654 | 1.00 | 62.19 | O |
| ATOM | 2733 | OD2 | ASP | A | 383 | 38.302 | −24.311 | −25.640 | 1.00 | 62.06 | O |
| ATOM | 2734 | C | ASP | A | 383 | 40.974 | −28.358 | −25.074 | 1.00 | 61.90 | C |
| ATOM | 2735 | O | ASP | A | 383 | 40.722 | −29.471 | −25.589 | 1.00 | 62.21 | O |
| ATOM | 2736 | N | ASP | A | 384 | 42.204 | −27.870 | −24.934 | 1.00 | 61.93 | N |
| ATOM | 2737 | CA | ASP | A | 384 | 43.377 | −28.633 | −25.329 | 1.00 | 62.20 | C |
| ATOM | 2738 | CB | ASP | A | 384 | 44.659 | −27.857 | −25.012 | 1.00 | 62.22 | C |
| ATOM | 2739 | CG | ASP | A | 384 | 45.905 | −28.552 | −25.527 | 1.00 | 62.42 | C |
| ATOM | 2740 | OD1 | ASP | A | 384 | 46.168 | −28.477 | −26.747 | 1.00 | 62.53 | O |
| ATOM | 2741 | OD2 | ASP | A | 384 | 46.625 | −29.168 | −24.711 | 1.00 | 62.70 | O |
| ATOM | 2742 | C | ASP | A | 384 | 43.387 | −29.985 | −24.621 | 1.00 | 62.27 | C |
| ATOM | 2743 | O | ASP | A | 384 | 43.630 | −31.022 | −25.261 | 1.00 | 62.50 | O |
| ATOM | 2744 | N | MET | A | 385 | 43.109 | −29.969 | −23.310 | 1.00 | 62.20 | N |
| ATOM | 2745 | CA | MET | A | 385 | 43.094 | −31.206 | −22.531 | 1.00 | 62.37 | C |
| ATOM | 2746 | CB | MET | A | 385 | 42.531 | −31.035 | −21.114 | 1.00 | 62.38 | C |
| ATOM | 2747 | CG | MET | A | 385 | 42.451 | −32.383 | −20.351 | 1.00 | 62.39 | C |
| ATOM | 2748 | SD | MET | A | 385 | 41.135 | −32.605 | −19.117 | 1.00 | 62.11 | S |
| ATOM | 2749 | CE | MET | A | 385 | 39.727 | −33.021 | −20.153 | 1.00 | 61.97 | C |
| ATOM | 2750 | C | MET | A | 385 | 42.244 | −32.216 | −23.244 | 1.00 | 62.48 | C |
| ATOM | 2751 | O | MET | A | 385 | 42.760 | −33.215 | −23.768 | 1.00 | 62.86 | O |
| ATOM | 2752 | N | LEU | A | 386 | 40.937 | −31.958 | −23.272 | 1.00 | 62.43 | N |
| ATOM | 2753 | CA | LEU | A | 386 | 40.058 | −32.982 | −23.813 | 1.00 | 62.76 | C |
| ATOM | 2754 | CB | LEU | A | 386 | 38.556 | −32.642 | −23.723 | 1.00 | 62.76 | C |
| ATOM | 2755 | CG | LEU | A | 386 | 37.940 | −31.254 | −23.928 | 1.00 | 62.69 | C |
| ATOM | 2756 | CD1 | LEU | A | 386 | 36.474 | −31.394 | −24.317 | 1.00 | 62.40 | C |
| ATOM | 2757 | CD2 | LEU | A | 386 | 38.072 | −30.425 | −22.665 | 1.00 | 62.82 | C |
| ATOM | 2758 | C | LEU | A | 386 | 40.515 | −33.334 | −25.217 | 1.00 | 62.86 | C |
| ATOM | 2759 | O | LEU | A | 386 | 40.936 | −34.480 | −25.461 | 1.00 | 63.18 | O |
| ATOM | 2760 | N | ALA | A | 387 | 40.506 | −32.335 | −26.104 | 1.00 | 62.84 | N |
| ATOM | 2761 | CA | ALA | A | 387 | 40.937 | −32.545 | −27.487 | 1.00 | 63.11 | C |
| ATOM | 2762 | CB | ALA | A | 387 | 41.213 | −31.207 | −28.168 | 1.00 | 62.92 | C |
| ATOM | 2763 | C | ALA | A | 387 | 42.163 | −33.465 | −27.585 | 1.00 | 63.24 | C |
| ATOM | 2764 | O | ALA | A | 387 | 42.042 | −34.694 | −27.776 | 1.00 | 63.61 | O |
| ATOM | 2765 | N | GLN | A | 388 | 43.343 | −32.877 | −27.416 | 1.00 | 63.14 | N |
| ATOM | 2766 | CA | GLN | A | 388 | 44.582 | −33.594 | −27.698 | 1.00 | 63.43 | C |
| ATOM | 2767 | CB | GLN | A | 388 | 45.744 | −32.603 | −27.824 | 1.00 | 63.49 | C |
| ATOM | 2768 | CG | GLN | A | 388 | 45.354 | −31.297 | −28.512 | 1.00 | 63.57 | C |
| ATOM | 2769 | CD | GLN | A | 388 | 46.408 | −30.790 | −29.481 | 1.00 | 63.61 | C |
| ATOM | 2770 | OE1 | GLN | A | 388 | 47.175 | −31.570 | −30.053 | 1.00 | 63.36 | O |
| ATOM | 2771 | NE2 | GLN | A | 388 | 46.441 | −29.477 | −29.681 | 1.00 | 63.79 | N |
| ATOM | 2772 | C | GLN | A | 388 | 44.906 | −34.688 | −26.676 | 1.00 | 63.52 | C |
| ATOM | 2773 | O | GLN | A | 388 | 45.198 | −35.850 | −27.041 | 1.00 | 63.82 | O |
| ATOM | 2774 | N | ASP | A | 389 | 44.844 | −34.323 | −25.397 | 1.00 | 63.47 | N |
| ATOM | 2775 | CA | ASP | A | 389 | 45.269 | −35.242 | −24.354 | 1.00 | 63.75 | C |
| ATOM | 2776 | CB | ASP | A | 389 | 45.274 | −34.549 | −22.992 | 1.00 | 63.76 | C |
| ATOM | 2777 | CG | ASP | A | 389 | 46.351 | −33.484 | −22.888 | 1.00 | 64.01 | C |
| ATOM | 2778 | OD1 | ASP | A | 389 | 47.535 | −33.801 | −23.137 | 1.00 | 64.41 | O |
| ATOM | 2779 | OD2 | ASP | A | 389 | 46.017 | −32.328 | −22.554 | 1.00 | 64.29 | O |
| ATOM | 2780 | C | ASP | A | 389 | 44.395 | −36.491 | −24.338 | 1.00 | 63.83 | C |
| ATOM | 2781 | O | ASP | A | 389 | 44.909 | −37.633 | −24.114 | 1.00 | 64.19 | O |
| ATOM | 2782 | N | ILE | A | 390 | 43.082 | −36.290 | −24.600 | 1.00 | 63.79 | N |
| ATOM | 2783 | CA | ILE | A | 390 | 42.202 | −37.449 | −24.564 | 1.00 | 64.00 | C |
| ATOM | 2784 | CB | ILE | A | 390 | 40.757 | −37.051 | −24.238 | 1.00 | 63.95 | C |
| ATOM | 2785 | CG1 | ILE | A | 390 | 40.670 | −36.707 | −22.748 | 1.00 | 63.71 | C |
| ATOM | 2786 | CD1 | ILE | A | 390 | 39.357 | −36.139 | −22.318 | 1.00 | 63.77 | C |
| ATOM | 2787 | CG2 | ILE | A | 390 | 39.789 | −38.171 | −24.600 | 1.00 | 63.97 | C |
| ATOM | 2788 | C | ILE | A | 390 | 42.349 | −38.215 | −25.875 | 1.00 | 64.14 | C |
| ATOM | 2789 | O | ILE | A | 390 | 42.347 | −39.465 | −25.896 | 1.00 | 64.58 | O |
| ATOM | 2790 | N | ALA | A | 391 | 42.530 | −37.458 | −26.958 | 1.00 | 64.14 | N |
| ATOM | 2791 | CA | ALA | A | 391 | 42.907 | −38.063 | −28.225 | 1.00 | 64.26 | C |
| ATOM | 2792 | CB | ALA | A | 391 | 43.364 | −37.001 | −29.210 | 1.00 | 64.25 | C |
| ATOM | 2793 | C | ALA | A | 391 | 43.998 | −39.120 | −28.018 | 1.00 | 64.38 | C |
| ATOM | 2794 | O | ALA | A | 391 | 43.828 | −40.265 | −28.427 | 1.00 | 64.34 | O |
| ATOM | 2795 | N | LYS | A | 392 | 45.100 | −38.750 | −27.361 | 1.00 | 64.51 | N |
| ATOM | 2796 | CA | LYS | A | 392 | 46.252 | −39.681 | −27.211 | 1.00 | 65.03 | C |
| ATOM | 2797 | CB | LYS | A | 392 | 47.541 | −38.900 | −26.844 | 1.00 | 65.00 | C |
| ATOM | 2798 | CG | LYS | A | 392 | 48.702 | −39.822 | −26.461 | 1.00 | 65.42 | C |
| ATOM | 2799 | CD | LYS | A | 392 | 49.804 | −39.084 | −25.687 | 1.00 | 65.51 | C |
| ATOM | 2800 | CE | LYS | A | 392 | 49.376 | −38.797 | −24.226 | 1.00 | 66.25 | C |
| ATOM | 2801 | NZ | LYS | A | 392 | 50.460 | −38.108 | −23.435 | 1.00 | 66.63 | N |
| ATOM | 2802 | C | LYS | A | 392 | 46.064 | −40.824 | −26.164 | 1.00 | 65.09 | C |
| ATOM | 2803 | O | LYS | A | 392 | 46.422 | −42.067 | −26.150 | 1.00 | 65.53 | O |
| ATOM | 2804 | N | LEU | A | 393 | 45.000 | −40.372 | −25.222 | 1.00 | 65.09 | N |
| ATOM | 2805 | CA | LEU | A | 393 | 44.770 | −41.413 | −24.203 | 1.00 | 65.41 | C |
| ATOM | 2806 | CB | LEU | A | 393 | 44.067 | −40.850 | −22.966 | 1.00 | 65.35 | C |
| ATOM | 2807 | CG | LEU | A | 393 | 45.072 | −40.402 | −21.908 | 1.00 | 65.48 | C |
| ATOM | 2808 | CD1 | LEU | A | 393 | 44.354 | −39.831 | −20.708 | 1.00 | 65.63 | C |
| ATOM | 2809 | CD2 | LEU | A | 393 | 45.954 | −41.576 | −21.502 | 1.00 | 65.50 | C |
| ATOM | 2810 | C | LEU | A | 393 | 43.969 | −42.559 | −24.811 | 1.00 | 65.64 | C |
| ATOM | 2811 | O | LEU | A | 393 | 44.037 | −43.692 | −24.318 | 1.00 | 65.90 | O |
| ATOM | 2812 | N | MET | A | 394 | 43.227 | −42.276 | −25.889 | 1.00 | 65.68 | N |
| ATOM | 2813 | CA | MET | A | 394 | 42.453 | −43.350 | −26.532 | 1.00 | 66.07 | C |
| ATOM | 2814 | CB | MET | A | 394 | 41.660 | −42.822 | −27.736 | 1.00 | 66.11 | C |
| ATOM | 2815 | CG | MET | A | 394 | 40.545 | −41.845 | −27.371 | 1.00 | 66.43 | C |
| ATOM | 2816 | SD | MET | A | 394 | 39.379 | −42.505 | −26.151 | 1.00 | 67.28 | S |
| ATOM | 2817 | CE | MET | A | 394 | 38.146 | −41.197 | −26.108 | 1.00 | 66.63 | C |
| ATOM | 2818 | C | MET | A | 394 | 43.334 | −44.552 | −26.932 | 1.00 | 66.21 | C |
| ATOM | 2819 | O | MET | A | 394 | 43.125 | −45.678 | −26.449 | 1.00 | 66.49 | O |
| ATOM | 2820 | N | PRO | A | 395 | 44.324 | −44.319 | −27.820 | 1.00 | 66.35 | N |
| ATOM | 2821 | CA | PRO | A | 395 | 45.221 | −45.400 | −28.246 | 1.00 | 66.36 | C |
| ATOM | 2822 | CB | PRO | A | 395 | 46.359 | −44.656 | −28.950 | 1.00 | 66.30 | C |
| ATOM | 2823 | CG | PRO | A | 395 | 45.708 | −43.450 | −29.505 | 1.00 | 66.32 | C |
| ATOM | 2824 | CD | PRO | A | 395 | 44.637 | −43.024 | −28.496 | 1.00 | 66.42 | C |
| ATOM | 2825 | C | PRO | A | 395 | 45.788 | −46.183 | −27.065 | 1.00 | 66.38 | C |
| ATOM | 2826 | O | PRO | A | 395 | 45.847 | −47.571 | −27.111 | 1.00 | 66.53 | O |
| ATOM | 2827 | N | LEU | A | 396 | 45.997 | −45.303 | −25.853 | 1.00 | 66.35 | N |
| ATOM | 2828 | CA | LEU | A | 396 | 46.746 | −46.083 | −24.821 | 1.00 | 66.51 | C |
| ATOM | 2829 | CB | LEU | A | 396 | 47.352 | −45.178 | −23.729 | 1.00 | 66.52 | C |
| ATOM | 2830 | CG | LEU | A | 396 | 48.412 | −45.758 | −22.772 | 1.00 | 66.50 | C |
| ATOM | 2831 | CD1 | LEU | A | 396 | 48.815 | −44.715 | −21.728 | 1.00 | 66.78 | C |
| ATOM | 2832 | CD2 | LEU | A | 396 | 47.966 | −47.051 | −22.076 | 1.00 | 66.41 | C |
| ATOM | 2833 | C | LEU | A | 396 | 45.701 | −47.020 | −24.189 | 1.00 | 66.56 | C |
| ATOM | 2834 | O | LEU | A | 396 | 45.970 | −48.217 | −23.789 | 1.00 | 66.72 | O |
| ATOM | 2835 | N | LEU | A | 397 | 44.490 | −46.430 | −24.125 | 1.00 | 66.57 | N |
| ATOM | 2836 | CA | LEU | A | 397 | 43.335 | −47.098 | −23.570 | 1.00 | 66.81 | C |
| ATOM | 2837 | CB | LEU | A | 397 | 42.107 | −46.199 | −23.695 | 1.00 | 66.77 | C |
| ATOM | 2838 | CG | LEU | A | 397 | 40.789 | −46.797 | −23.217 | 1.00 | 66.74 | C |
| ATOM | 2839 | CD1 | LEU | A | 397 | 40.828 | −47.016 | −21.711 | 1.00 | 66.79 | C |
| ATOM | 2840 | CD2 | LEU | A | 397 | 39.643 | −45.884 | −23.609 | 1.00 | 66.83 | C |
| ATOM | 2841 | C | LEU | A | 397 | 43.082 | −48.420 | −24.267 | 1.00 | 66.95 | C |
| ATOM | 2842 | O | LEU | A | 397 | 42.883 | −49.426 | −23.605 | 1.00 | 67.22 | O |
| ATOM | 2843 | N | ARG | A | 398 | 43.092 | −48.416 | −25.599 | 1.00 | 67.02 | N |
| ATOM | 2844 | CA | ARG | A | 398 | 42.836 | −49.643 | −26.369 | 1.00 | 67.30 | C |
| ATOM | 2845 | CB | ARG | A | 398 | 42.850 | −49.332 | −27.868 | 1.00 | 67.36 | C |
| ATOM | 2846 | CG | ARG | A | 398 | 41.808 | −48.309 | −28.301 | 1.00 | 67.98 | C |
| ATOM | 2847 | CD | ARG | A | 398 | 42.319 | −47.456 | −29.459 | 1.00 | 68.97 | C |
| ATOM | 2848 | NE | ARG | A | 398 | 42.794 | −48.259 | −30.583 | 1.00 | 69.67 | N |
| ATOM | 2849 | CZ | ARG | A | 398 | 42.005 | −48.760 | −31.532 | 1.00 | 70.33 | C |
| ATOM | 2850 | NH1 | ARG | A | 398 | 42.528 | −49.478 | −32.520 | 1.00 | 70.67 | N |
| ATOM | 2851 | NH2 | ARG | A | 398 | 40.693 | −48.547 | −31.494 | 1.00 | 70.46 | N |
| ATOM | 2852 | C | ARG | A | 398 | 43.866 | −50.744 | −26.069 | 1.00 | 67.28 | C |
| ATOM | 2853 | O | ARG | A | 398 | 43.504 | −52.040 | −25.930 | 1.00 | 67.55 | O |
| ATOM | 2854 | N | GLN | A | 399 | 45.149 | −50.229 | −25.856 | 1.00 | 67.15 | N |
| ATOM | 2855 | CA | GLN | A | 399 | 46.206 | −51.190 | −25.539 | 1.00 | 67.22 | C |
| ATOM | 2856 | CB | GLN | A | 399 | 47.589 | −50.529 | −25.575 | 1.00 | 67.21 | C |
| ATOM | 2857 | CG | GLN | A | 399 | 48.756 | −51.484 | −25.318 | 1.00 | 67.20 | C |
| ATOM | 2858 | CD | GLN | A | 399 | 48.755 | −52.683 | −26.261 | 1.00 | 67.24 | C |
| ATOM | 2859 | OE1 | GLN | A | 399 | 48.822 | −53.832 | −25.813 | 1.00 | 67.09 | O |
| ATOM | 2860 | NE2 | GLN | A | 399 | 48.674 | −52.421 | −27.571 | 1.00 | 67.33 | N |
| ATOM | 2861 | C | GLN | A | 399 | 45.927 | −51.801 | −24.170 | 1.00 | 67.26 | C |
| ATOM | 2862 | O | GLN | A | 399 | 46.229 | −52.973 | −23.925 | 1.00 | 67.20 | O |
| ATOM | 2863 | N | GLU | A | 400 | 45.333 | −51.006 | −23.282 | 1.00 | 67.33 | N |
| ATOM | 2864 | CA | GLU | A | 400 | 44.948 | −51.520 | −21.970 | 1.00 | 67.67 | C |
| ATOM | 2865 | CB | GLU | A | 400 | 44.770 | −50.383 | −20.966 | 1.00 | 67.73 | C |
| ATOM | 2866 | CG | GLU | A | 400 | 46.081 | −49.853 | −20.396 | 1.00 | 68.29 | C |
| ATOM | 2867 | CD | GLU | A | 400 | 45.856 | −49.020 | −19.145 | 1.00 | 68.96 | C |
| ATOM | 2868 | OE1 | GLU | A | 400 | 46.798 | −48.311 | −18.724 | 1.00 | 69.08 | O |
| ATOM | 2869 | OE2 | GLU | A | 400 | 44.735 | −49.077 | −18.586 | 1.00 | 69.03 | O |
| ATOM | 2870 | C | GLU | A | 400 | 43.681 | −52.374 | −22.016 | 1.00 | 67.75 | C |
| ATOM | 2871 | O | GLU | A | 400 | 43.505 | −53.276 | −21.197 | 1.00 | 67.75 | O |
| ATOM | 2872 | N | GLU | A | 401 | 42.802 | −52.089 | −22.974 | 1.00 | 67.83 | N |
| ATOM | 2873 | CA | GLU | A | 401 | 41.535 | −52.807 | −23.088 | 1.00 | 68.15 | C |
| ATOM | 2874 | CB | GLU | A | 401 | 40.515 | −52.036 | −23.945 | 1.00 | 68.25 | C |
| ATOM | 2875 | CG | GLU | A | 401 | 39.080 | −52.606 | −23.881 | 1.00 | 68.55 | C |
| ATOM | 2876 | CD | GLU | A | 401 | 38.001 | −51.549 | −24.104 | 1.00 | 68.79 | C |
| ATOM | 2877 | OE1 | GLU | A | 401 | 38.129 | −50.435 | −23.549 | 1.00 | 68.79 | O |
| ATOM | 2878 | OE2 | GLU | A | 401 | 37.017 | −51.839 | −24.824 | 1.00 | 68.49 | O |
| ATOM | 2879 | C | GLU | A | 401 | 41.775 | −54.218 | −23.626 | 1.00 | 68.19 | C |
| ATOM | 2880 | O | GLU | A | 401 | 40.863 | −55.103 | −23.541 | 1.00 | 68.20 | O |
| ATOM | 2881 | N | LEU | A | 402 | 43.014 | −54.443 | −24.161 | 1.00 | 68.10 | N |
| ATOM | 2882 | CA | LEU | A | 402 | 43.360 | −55.876 | −24.349 | 1.00 | 68.26 | C |
| ATOM | 2883 | CB | LEU | A | 402 | 43.651 | −56.248 | −25.818 | 1.00 | 68.22 | C |
| ATOM | 2884 | CG | LEU | A | 402 | 44.480 | −55.386 | −26.774 | 1.00 | 68.29 | C |
| ATOM | 2885 | CD1 | LEU | A | 402 | 45.951 | −55.301 | −26.369 | 1.00 | 68.15 | C |
| ATOM | 2886 | CD2 | LEU | A | 402 | 44.340 | −55.954 | −28.185 | 1.00 | 68.27 | C |
| ATOM | 2887 | C | LEU | A | 402 | 44.480 | −56.322 | −23.386 | 1.00 | 68.35 | C |
| ATOM | 2888 | O | LEU | A | 402 | 45.647 | −56.557 | −23.789 | 1.00 | 68.28 | O |
| ATOM | 2889 | N | GLU | A | 403 | 44.107 | −56.428 | −22.102 | 1.00 | 68.52 | N |
| ATOM | 2890 | CA | GLU | A | 403 | 45.088 | −56.755 | −21.065 | 1.00 | 68.65 | C |
| ATOM | 2891 | CB | GLU | A | 403 | 45.431 | −55.521 | −20.224 | 1.00 | 68.70 | C |
| ATOM | 2892 | CG | GLU | A | 403 | 46.491 | −54.639 | −20.862 | 1.00 | 68.74 | C |
| ATOM | 2893 | CD | GLU | A | 403 | 47.715 | −55.426 | −21.293 | 1.00 | 68.53 | C |
| ATOM | 2894 | OE1 | GLU | A | 403 | 47.701 | −56.670 | −21.161 | 1.00 | 68.11 | O |
| ATOM | 2895 | OE2 | GLU | A | 403 | 48.691 | −54.802 | −21.761 | 1.00 | 68.50 | O |
| ATOM | 2896 | C | GLU | A | 403 | 44.714 | −57.924 | −20.161 | 1.00 | 68.69 | C |
| ATOM | 2897 | O | GLU | A | 403 | 45.249 | −59.023 | −20.320 | 1.00 | 68.78 | O |
| ATOM | 2898 | N | SER | A | 404 | 43.817 | −57.699 | −19.204 | 1.00 | 68.70 | N |
| ATOM | 2899 | CA | SER | A | 404 | 43.474 | −58.768 | −18.266 | 1.00 | 68.71 | C |
| ATOM | 2900 | CB | SER | A | 404 | 44.240 | −58.599 | −16.944 | 1.00 | 68.72 | C |
| ATOM | 2901 | OG | SER | A | 404 | 44.441 | −59.853 | −16.305 | 1.00 | 68.64 | O |
| ATOM | 2902 | C | SER | A | 404 | 41.970 | −58.933 | −18.010 | 1.00 | 68.68 | C |
| ATOM | 2903 | O | SER | A | 404 | 41.344 | −58.123 | −17.318 | 1.00 | 68.72 | O |
| ATOM | 2904 | N | VAL | A | 405 | 41.405 | −59.999 | −18.576 | 1.00 | 68.52 | N |
| ATOM | 2905 | CA | VAL | A | 405 | 40.014 | −60.373 | −18.330 | 1.00 | 68.31 | C |
| ATOM | 2906 | CB | VAL | A | 405 | 39.515 | −61.401 | −19.382 | 1.00 | 68.37 | C |
| ATOM | 2907 | CG1 | VAL | A | 405 | 38.017 | −61.661 | −19.226 | 1.00 | 68.42 | C |
| ATOM | 2908 | CG2 | VAL | A | 405 | 40.316 | −62.702 | −19.301 | 1.00 | 68.30 | C |
| ATOM | 2909 | C | VAL | A | 405 | 39.859 | −60.941 | −16.914 | 1.00 | 68.09 | C |
| ATOM | 2910 | O | VAL | A | 405 | 38.742 | −61.172 | −16.440 | 1.00 | 68.12 | O |
| ATOM | 2911 | N | GLU | A | 406 | 40.994 | −61.154 | −16.249 | 1.00 | 67.77 | N |
| ATOM | 2912 | CA | GLU | A | 406 | 41.041 | −61.660 | −14.875 | 1.00 | 67.37 | C |
| ATOM | 2913 | CB | GLU | A | 406 | 42.492 | −61.732 | −14.388 | 1.00 | 67.50 | C |
| ATOM | 2914 | CG | GLU | A | 406 | 42.647 | −62.138 | −12.926 | 1.00 | 67.80 | C |
| ATOM | 2915 | CD | GLU | A | 406 | 43.569 | −61.202 | −12.160 | 1.00 | 68.31 | C |
| ATOM | 2916 | OE1 | GLU | A | 406 | 44.561 | −61.685 | −11.570 | 1.00 | 68.29 | O |
| ATOM | 2917 | OE2 | GLU | A | 406 | 43.301 | −59.978 | −12.151 | 1.00 | 68.51 | O |
| ATOM | 2918 | C | GLU | A | 406 | 40.234 | −60.777 | −13.926 | 1.00 | 66.93 | C |
| ATOM | 2919 | O | GLU | A | 406 | 39.662 | −61.265 | −12.946 | 1.00 | 66.98 | O |
| ATOM | 2920 | N | ALA | A | 407 | 40.197 | −59.479 | −14.224 | 1.00 | 66.23 | N |
| ATOM | 2921 | CA | ALA | A | 407 | 39.443 | −58.516 | −13.424 | 1.00 | 65.47 | C |
| ATOM | 2922 | CB | ALA | A | 407 | 39.721 | −57.094 | −13.907 | 1.00 | 65.49 | C |
| ATOM | 2923 | C | ALA | A | 407 | 37.940 | −58.813 | −13.441 | 1.00 | 64.85 | C |
| ATOM | 2924 | O | ALA | A | 407 | 37.202 | −58.369 | −12.555 | 1.00 | 64.84 | O |
| ATOM | 2925 | N | GLY | A | 408 | 37.504 | −59.576 | −14.444 | 1.00 | 64.00 | N |
| ATOM | 2926 | CA | GLY | A | 408 | 36.098 | −59.936 | −14.617 | 1.00 | 62.91 | C |
| ATOM | 2927 | C | GLY | A | 408 | 35.433 | −60.428 | −13.348 | 1.00 | 62.16 | C |
| ATOM | 2928 | O | GLY | A | 408 | 36.082 | −61.020 | −12.486 | 1.00 | 62.11 | O |
| ATOM | 2929 | N | VAL | A | 409 | 34.133 | −60.180 | −13.234 | 1.00 | 61.40 | N |
| ATOM | 2930 | CA | VAL | A | 409 | 33.380 | −60.595 | −12.059 | 1.00 | 60.66 | C |
| ATOM | 2931 | CB | VAL | A | 409 | 31.952 | −60.013 | −12.059 | 1.00 | 60.68 | C |
| ATOM | 2932 | CG1 | VAL | A | 409 | 31.345 | −60.103 | −10.670 | 1.00 | 60.53 | C |
| ATOM | 2933 | CG2 | VAL | A | 409 | 31.973 | −58.573 | −12.521 | 1.00 | 60.66 | C |
| ATOM | 2934 | C | VAL | A | 409 | 33.329 | −62.117 | −11.964 | 1.00 | 60.21 | C |
| ATOM | 2935 | O | VAL | A | 409 | 32.927 | −62.803 | −12.909 | 1.00 | 60.00 | O |
| ATOM | 2936 | N | ALA | A | 410 | 33.750 | −62.638 | −10.817 | 1.00 | 59.67 | N |
| ATOM | 2937 | CA | ALA | A | 410 | 33.824 | −64.077 | −10.612 | 1.00 | 59.13 | C |
| ATOM | 2938 | CB | ALA | A | 410 | 35.019 | −64.425 | −9.741 | 1.00 | 59.26 | C |
| ATOM | 2939 | C | ALA | A | 410 | 32.543 | −64.616 | −9.998 | 1.00 | 58.71 | C |
| ATOM | 2940 | O | ALA | A | 410 | 31.774 | −63.875 | −9.392 | 1.00 | 58.61 | O |
| ATOM | 2941 | N | GLY | A | 411 | 32.338 | −65.919 | −10.160 | 1.00 | 58.33 | N |
| ATOM | 2942 | CA | GLY | A | 411 | 31.135 | −66.617 | −9.709 | 1.00 | 57.74 | C |
| ATOM | 2943 | C | GLY | A | 411 | 30.218 | −65.930 | −8.713 | 1.00 | 57.25 | C |
| ATOM | 2944 | O | GLY | A | 411 | 30.664 | −65.273 | −7.770 | 1.00 | 57.29 | O |
| ATOM | 2945 | N | GLY | A | 412 | 28.920 | −66.103 | −8.937 | 1.00 | 56.81 | N |
| ATOM | 2946 | CA | GLY | A | 412 | 27.885 | −65.639 | −8.023 | 1.00 | 56.24 | C |
| ATOM | 2947 | C | GLY | A | 412 | 26.593 | −66.100 | −8.654 | 1.00 | 55.88 | C |
| ATOM | 2948 | O | GLY | A | 412 | 26.023 | −67.115 | −8.267 | 1.00 | 55.87 | O |
| ATOM | 2949 | N | ALA | A | 413 | 26.149 | −65.349 | −9.651 | 1.00 | 55.53 | N |
| ATOM | 2950 | CA | ALA | A | 413 | 25.119 | −65.806 | −10.564 | 1.00 | 55.19 | C |
| ATOM | 2951 | CB | ALA | A | 413 | 23.952 | −64.844 | −10.570 | 1.00 | 55.14 | C |
| ATOM | 2952 | C | ALA | A | 413 | 25.763 | −65.865 | −11.937 | 1.00 | 54.97 | C |
| ATOM | 2953 | O | ALA | A | 413 | 25.107 | −66.140 | −12.938 | 1.00 | 55.00 | O |
| ATOM | 2954 | N | PHE | A | 414 | 27.066 | −65.613 | −11.968 | 1.00 | 54.73 | N |
| ATOM | 2955 | CA | PHE | A | 414 | 27.791 | −65.468 | −13.220 | 1.00 | 54.57 | C |
| ATOM | 2956 | CB | PHE | A | 414 | 28.869 | −64.387 | −13.090 | 1.00 | 54.59 | C |
| ATOM | 2957 | CG | PHE | A | 414 | 28.326 | −63.027 | −12.744 | 1.00 | 54.41 | C |
| ATOM | 2958 | CD1 | PHE | A | 414 | 27.851 | −62.184 | −13.735 | 1.00 | 54.25 | C |
| ATOM | 2959 | CE1 | PHE | A | 414 | 27.351 | −60.941 | −13.421 | 1.00 | 54.27 | C |
| ATOM | 2960 | CZ | PHE | A | 414 | 27.322 | −60.521 | −12.105 | 1.00 | 54.38 | C |
| ATOM | 2961 | CE2 | PHE | A | 414 | 27.792 | −61.350 | −11.109 | 1.00 | 54.20 | C |
| ATOM | 2962 | CD2 | PHE | A | 414 | 28.291 | −62.594 | −11.429 | 1.00 | 54.24 | C |
| ATOM | 2963 | C | PHE | A | 414 | 28.421 | −66.771 | −13.689 | 1.00 | 54.51 | C |
| ATOM | 2964 | O | PHE | A | 414 | 28.536 | −67.005 | −14.892 | 1.00 | 54.59 | O |
| ATOM | 2965 | N | GLU | A | 415 | 28.827 | −67.612 | −12.740 | 1.00 | 54.32 | N |
| ATOM | 2966 | CA | GLU | A | 415 | 29.498 | −68.872 | −13.056 | 1.00 | 54.13 | C |
| ATOM | 2967 | CB | GLU | A | 415 | 29.668 | −69.713 | −11.796 | 1.00 | 54.25 | C |
| ATOM | 2968 | CG | GLU | A | 415 | 28.358 | −70.145 | −11.184 | 1.00 | 55.37 | C |
| ATOM | 2969 | CD | GLU | A | 415 | 28.459 | −70.379 | −9.691 | 1.00 | 57.16 | C |
| ATOM | 2970 | OE1 | GLU | A | 415 | 27.487 | −70.912 | −9.105 | 1.00 | 57.92 | O |
| ATOM | 2971 | OE2 | GLU | A | 415 | 29.507 | −70.025 | −9.104 | 1.00 | 57.82 | O |
| ATOM | 2972 | C | GLU | A | 415 | 28.758 | −69.664 | −14.133 | 1.00 | 53.71 | C |
| ATOM | 2973 | O | GLU | A | 415 | 27.527 | −69.672 | −14.179 | 1.00 | 53.58 | O |
| ATOM | 2974 | N | GLY | A | 416 | 29.523 | −70.325 | −14.997 | 1.00 | 53.38 | N |
| ATOM | 2975 | CA | GLY | A | 416 | 28.966 | −70.999 | −16.165 | 1.00 | 52.98 | C |
| ATOM | 2976 | C | GLY | A | 416 | 28.302 | −72.320 | −15.844 | 1.00 | 52.72 | C |
| ATOM | 2977 | O | GLY | A | 416 | 27.353 | −72.729 | −16.516 | 1.00 | 52.60 | O |
| ATOM | 2978 | N | THR | A | 417 | 28.805 | −72.985 | −14.808 | 1.00 | 52.54 | N |
| ATOM | 2979 | CA | THR | A | 417 | 28.274 | −74.275 | −14.382 | 1.00 | 52.37 | C |
| ATOM | 2980 | CB | THR | A | 417 | 29.094 | −74.865 | −13.219 | 1.00 | 52.27 | C |
| ATOM | 2981 | OG1 | THR | A | 417 | 29.438 | −73.824 | −12.299 | 1.00 | 51.98 | O |
| ATOM | 2982 | CG2 | THR | A | 417 | 30.369 | −75.497 | −13.736 | 1.00 | 52.29 | C |
| ATOM | 2983 | C | THR | A | 417 | 26.803 | −74.199 | −13.977 | 1.00 | 52.41 | C |
| ATOM | 2984 | O | THR | A | 417 | 26.166 | −75.226 | −13.746 | 1.00 | 52.44 | O |
| ATOM | 2985 | N | ARG | A | 418 | 26.269 | −72.982 | −13.899 | 1.00 | 52.48 | N |
| ATOM | 2986 | CA | ARG | A | 418 | 24.876 | −72.768 | −13.518 | 1.00 | 52.60 | C |
| ATOM | 2987 | CB | ARG | A | 418 | 24.659 | −71.335 | −13.031 | 1.00 | 52.61 | C |
| ATOM | 2988 | CG | ARG | A | 418 | 25.100 | −71.097 | −11.597 | 1.00 | 53.27 | C |
| ATOM | 2989 | CD | ARG | A | 418 | 24.677 | −69.722 | −11.083 | 1.00 | 54.81 | C |
| ATOM | 2990 | NE | ARG | A | 418 | 23.237 | −69.623 | −10.850 | 1.00 | 55.96 | N |
| ATOM | 2991 | CZ | ARG | A | 418 | 22.371 | −69.084 | −11.706 | 1.00 | 56.80 | C |
| ATOM | 2992 | NH1 | ARG | A | 418 | 21.077 | −69.038 | −11.405 | 1.00 | 57.11 | N |
| ATOM | 2993 | NH2 | ARG | A | 418 | 22.795 | −68.589 | −12.864 | 1.00 | 56.93 | N |
| ATOM | 2994 | C | ARG | A | 418 | 23.916 | −73.077 | −14.657 | 1.00 | 52.65 | C |
| ATOM | 2995 | O | ARG | A | 418 | 22.722 | −73.271 | −14.439 | 1.00 | 52.59 | O |
| ATOM | 2996 | N | MET | A | 419 | 24.443 | −73.129 | −15.873 | 1.00 | 52.86 | N |
| ATOM | 2997 | CA | MET | A | 419 | 23.613 | −73.399 | −17.037 | 1.00 | 53.06 | C |
| ATOM | 2998 | CB | MET | A | 419 | 24.176 | −72.685 | −18.273 | 1.00 | 53.29 | C |
| ATOM | 2999 | CG | MET | A | 419 | 23.147 | −72.383 | −19.370 | 1.00 | 54.17 | C |
| ATOM | 3000 | SD | MET | A | 419 | 21.616 | −71.599 | −18.788 | 1.00 | 55.92 | S |
| ATOM | 3001 | CE | MET | A | 419 | 20.494 | −72.997 | −18.721 | 1.00 | 55.37 | C |
| ATOM | 3002 | C | MET | A | 419 | 23.462 | −74.904 | −17.280 | 1.00 | 52.89 | C |
| ATOM | 3003 | O | MET | A | 419 | 22.750 | −75.326 | −18.190 | 1.00 | 52.78 | O |
| ATOM | 3004 | N | GLY | A | 420 | 24.124 | −75.707 | −16.452 | 1.00 | 52.86 | N |
| ATOM | 3005 | CA | GLY | A | 420 | 24.028 | −77.161 | −16.549 | 1.00 | 52.96 | C |
| ATOM | 3006 | C | GLY | A | 420 | 25.303 | −77.821 | −17.045 | 1.00 | 53.03 | C |
| ATOM | 3007 | O | GLY | A | 420 | 26.231 | −77.138 | −17.476 | 1.00 | 53.13 | O |
| ATOM | 3008 | N | PRO | A | 421 | 25.364 | −79.161 | −16.972 | 1.00 | 53.04 | N |
| ATOM | 3009 | CA | PRO | A | 421 | 26.537 | −79.878 | −17.412 | 1.00 | 53.09 | C |
| ATOM | 3010 | CB | PRO | A | 421 | 26.673 | −80.952 | −16.335 | 1.00 | 52.99 | C |
| ATOM | 3011 | CG | PRO | A | 421 | 25.217 | −81.301 | −15.998 | 1.00 | 52.91 | C |
| ATOM | 3012 | CD | PRO | A | 421 | 24.357 | −80.087 | −16.418 | 1.00 | 53.05 | C |
| ATOM | 3013 | C | PRO | A | 421 | 26.348 | −80.558 | −18.768 | 1.00 | 53.26 | C |
| ATOM | 3014 | O | PRO | A | 421 | 27.242 | −81.294 | −19.215 | 1.00 | 53.35 | O |
| ATOM | 3015 | N | PHE | A | 422 | 25.210 | −80.321 | −19.424 | 1.00 | 53.47 | N |
| ATOM | 3016 | CA | PHE | A | 422 | 24.861 | −81.125 | −20.599 | 1.00 | 53.71 | C |
| ATOM | 3017 | CB | PHE | A | 422 | 23.439 | −81.673 | −20.491 | 1.00 | 53.51 | C |
| ATOM | 3018 | CG | PHE | A | 422 | 23.360 | −82.947 | −19.722 | 1.00 | 53.01 | C |
| ATOM | 3019 | CD1 | PHE | A | 422 | 23.056 | −82.939 | −18.377 | 1.00 | 52.78 | C |
| ATOM | 3020 | CE1 | PHE | A | 422 | 23.000 | −84.113 | −17.662 | 1.00 | 52.83 | C |
| ATOM | 3021 | CZ | PHE | A | 422 | 23.260 | −85.312 | −18.287 | 1.00 | 52.98 | C |
| ATOM | 3022 | CE2 | PHE | A | 422 | 23.577 | −85.331 | −19.627 | 1.00 | 52.93 | C |
| ATOM | 3023 | CD2 | PHE | A | 422 | 23.631 | −84.153 | −20.336 | 1.00 | 52.79 | C |
| ATOM | 3024 | C | PHE | A | 422 | 25.106 | −80.504 | −21.970 | 1.00 | 54.16 | C |
| ATOM | 3025 | O | PHE | A | 422 | 25.548 | −81.193 | −22.894 | 1.00 | 54.20 | O |
| ATOM | 3026 | N | VAL | A | 423 | 24.814 | −79.217 | −22.116 | 1.00 | 54.72 | N |
| ATOM | 3027 | CA | VAL | A | 423 | 25.103 | −78.540 | −23.376 | 1.00 | 55.30 | C |
| ATOM | 3028 | CB | VAL | A | 423 | 23.829 | −77.988 | −24.055 | 1.00 | 55.35 | C |
| ATOM | 3029 | CG1 | VAL | A | 423 | 24.187 | −77.283 | −25.358 | 1.00 | 55.52 | C |
| ATOM | 3030 | CG2 | VAL | A | 423 | 22.840 | −79.119 | −24.324 | 1.00 | 55.43 | C |
| ATOM | 3031 | C | VAL | A | 423 | 26.133 | −77.434 | −23.179 | 1.00 | 55.57 | C |
| ATOM | 3032 | O | VAL | A | 423 | 25.864 | −76.444 | −22.496 | 1.00 | 55.56 | O |
| ATOM | 3033 | N | GLU | A | 424 | 27.308 | −77.627 | −23.783 | 1.00 | 56.06 | N |
| ATOM | 3034 | CA | GLU | A | 424 | 28.436 | −76.685 | −23.709 | 1.00 | 56.34 | C |
| ATOM | 3035 | CB | GLU | A | 424 | 28.105 | −75.352 | −24.420 | 1.00 | 56.44 | C |
| ATOM | 3036 | CG | GLU | A | 424 | 29.293 | −74.350 | −24.581 | 1.00 | 56.50 | C |
| ATOM | 3037 | CD | GLU | A | 424 | 29.181 | −73.133 | −23.625 | 1.00 | 57.30 | C |
| ATOM | 3038 | OE1 | GLU | A | 424 | 27.963 | −72.749 | −23.245 | 1.00 | 57.51 | O |
| ATOM | 3039 | OE2 | GLU | A | 424 | 30.250 | −72.545 | −23.247 | 1.00 | 57.40 | O |
| ATOM | 3040 | C | GLU | A | 424 | 28.929 | −76.440 | −22.273 | 1.00 | 56.48 | C |
| ATOM | 3041 | O | GLU | A | 424 | 28.279 | −75.721 | −21.495 | 1.00 | 56.64 | O |
| ATOM | 3042 | N | GLU | A | 443 | 15.905 | −85.521 | −51.072 | 1.00 | 65.60 | N |
| ATOM | 3043 | CA | GLU | A | 443 | 14.889 | −84.482 | −51.179 | 1.00 | 65.73 | C |
| ATOM | 3044 | CB | GLU | A | 443 | 14.210 | −84.527 | −52.553 | 1.00 | 66.04 | C |
| ATOM | 3045 | CG | GLU | A | 443 | 13.123 | −83.462 | −52.765 | 1.00 | 67.13 | C |
| ATOM | 3046 | CD | GLU | A | 443 | 13.687 | −82.071 | −53.033 | 1.00 | 68.58 | C |
| ATOM | 3047 | OE1 | GLU | A | 443 | 14.867 | −81.816 | −52.699 | 1.00 | 69.01 | O |
| ATOM | 3048 | OE2 | GLU | A | 443 | 12.942 | −81.229 | −53.583 | 1.00 | 69.27 | O |
| ATOM | 3049 | C | GLU | A | 443 | 13.847 | −84.609 | −50.074 | 1.00 | 65.36 | C |
| ATOM | 3050 | O | GLU | A | 443 | 14.126 | −84.292 | −48.918 | 1.00 | 65.51 | O |
| ATOM | 3051 | N | TRP | A | 444 | 12.652 | −85.071 | −50.441 | 1.00 | 64.83 | N |
| ATOM | 3052 | CA | TRP | A | 444 | 11.546 | −85.228 | −49.500 | 1.00 | 64.28 | C |
| ATOM | 3053 | CB | TRP | A | 444 | 10.269 | −84.611 | −50.076 | 1.00 | 64.11 | C |
| ATOM | 3054 | CG | TRP | A | 444 | 9.181 | −84.393 | −49.063 | 1.00 | 63.96 | C |
| ATOM | 3055 | CD1 | TRP | A | 444 | 9.343 | −84.086 | −47.744 | 1.00 | 63.74 | C |
| ATOM | 3056 | NE1 | TRP | A | 444 | 8.118 | −83.952 | −47.135 | 1.00 | 63.50 | N |
| ATOM | 3057 | CE2 | TRP | A | 444 | 7.133 | −84.164 | −48.062 | 1.00 | 63.49 | C |
| ATOM | 3058 | CD2 | TRP | A | 444 | 7.764 | −84.438 | −49.292 | 1.00 | 63.81 | C |
| ATOM | 3059 | CE3 | TRP | A | 444 | 6.970 | −84.694 | −50.414 | 1.00 | 63.77 | C |
| ATOM | 3060 | CZ3 | TRP | A | 444 | 5.591 | −84.665 | −50.271 | 1.00 | 63.70 | C |
| ATOM | 3061 | CH2 | TRP | A | 444 | 4.995 | −84.386 | −49.037 | 1.00 | 63.62 | C |
| ATOM | 3062 | CZ2 | TRP | A | 444 | 5.747 | −84.137 | −47.923 | 1.00 | 63.59 | C |
| ATOM | 3063 | C | TRP | A | 444 | 11.342 | −86.707 | −49.211 | 1.00 | 64.04 | C |
| ATOM | 3064 | O | TRP | A | 444 | 11.421 | −87.531 | −50.116 | 1.00 | 64.09 | O |
| ATOM | 3065 | N | VAL | A | 445 | 11.074 | −87.043 | −47.953 | 1.00 | 63.74 | N |
| ATOM | 3066 | CA | VAL | A | 445 | 11.078 | −88.445 | −47.534 | 1.00 | 63.47 | C |
| ATOM | 3067 | CB | VAL | A | 445 | 11.343 | −88.591 | −46.016 | 1.00 | 63.49 | C |
| ATOM | 3068 | CG1 | VAL | A | 445 | 12.100 | −89.891 | −45.746 | 1.00 | 63.40 | C |
| ATOM | 3069 | CG2 | VAL | A | 445 | 12.153 | −87.410 | −45.501 | 1.00 | 63.67 | C |
| ATOM | 3070 | C | VAL | A | 445 | 9.814 | −89.236 | −47.888 | 1.00 | 63.28 | C |
| ATOM | 3071 | O | VAL | A | 445 | 9.848 | −90.466 | −47.930 | 1.00 | 63.22 | O |
| ATOM | 3072 | N | VAL | A | 446 | 8.703 | −88.549 | −48.141 | 1.00 | 63.03 | N |
| ATOM | 3073 | CA | VAL | A | 446 | 7.432 | −89.251 | −48.345 | 1.00 | 62.82 | C |
| ATOM | 3074 | CB | VAL | A | 446 | 6.242 | −88.540 | −47.646 | 1.00 | 62.73 | C |
| ATOM | 3075 | CG1 | VAL | A | 446 | 6.725 | −87.739 | −46.447 | 1.00 | 62.74 | C |
| ATOM | 3076 | CG2 | VAL | A | 446 | 5.508 | −87.644 | −48.612 | 1.00 | 62.62 | C |
| ATOM | 3077 | C | VAL | A | 446 | 7.100 | −89.527 | −49.817 | 1.00 | 62.78 | C |
| ATOM | 3078 | O | VAL | A | 446 | 6.157 | −90.263 | −50.119 | 1.00 | 62.73 | O |
| ATOM | 3079 | N | THR | A | 447 | 7.884 | −88.954 | −50.728 | 1.00 | 62.71 | N |
| ATOM | 3080 | CA | THR | A | 447 | 7.650 | −89.148 | −52.161 | 1.00 | 62.74 | C |
| ATOM | 3081 | CB | THR | A | 447 | 8.638 | −88.339 | −53.025 | 1.00 | 62.72 | C |
| ATOM | 3082 | OG1 | THR | A | 447 | 9.861 | −88.163 | −52.305 | 1.00 | 63.01 | O |
| ATOM | 3083 | CG2 | THR | A | 447 | 8.062 | −86.972 | −53.357 | 1.00 | 62.66 | C |
| ATOM | 3084 | C | THR | A | 447 | 7.680 | −90.620 | −52.582 | 1.00 | 62.71 | C |
| ATOM | 3085 | O | THR | A | 447 | 7.073 | −90.991 | −53.588 | 1.00 | 62.63 | O |
| ATOM | 3086 | N | LYS | A | 448 | 8.379 | −91.453 | −51.813 | 1.00 | 62.74 | N |
| ATOM | 3087 | CA | LYS | A | 448 | 8.381 | −92.895 | −52.061 | 1.00 | 62.80 | C |
| ATOM | 3088 | CB | LYS | A | 448 | 9.370 | −93.628 | −51.148 | 1.00 | 62.76 | C |
| ATOM | 3089 | CG | LYS | A | 448 | 10.789 | −93.108 | −51.197 | 1.00 | 63.05 | C |
| ATOM | 3090 | CD | LYS | A | 448 | 11.108 | −92.289 | −49.960 | 1.00 | 63.48 | C |
| ATOM | 3091 | CE | LYS | A | 448 | 12.245 | −91.319 | −50.222 | 1.00 | 63.64 | C |
| ATOM | 3092 | NZ | LYS | A | 448 | 11.832 | −90.275 | −51.202 | 1.00 | 63.53 | N |
| ATOM | 3093 | C | LYS | A | 448 | 6.986 | −93.490 | −51.874 | 1.00 | 62.83 | C |
| ATOM | 3094 | O | LYS | A | 448 | 6.543 | −94.314 | −52.674 | 1.00 | 63.01 | O |
| ATOM | 3095 | N | ASP | A | 449 | 6.297 | −93.070 | −50.818 | 1.00 | 62.70 | N |
| ATOM | 3096 | CA | ASP | A | 449 | 4.993 | −93.632 | −50.493 | 1.00 | 62.64 | C |
| ATOM | 3097 | CB | ASP | A | 449 | 4.882 | −93.837 | −48.981 | 1.00 | 62.80 | C |
| ATOM | 3098 | CG | ASP | A | 449 | 5.951 | −94.782 | −48.439 | 1.00 | 63.19 | C |
| ATOM | 3099 | OD1 | ASP | A | 449 | 6.122 | −95.890 | −49.006 | 1.00 | 63.74 | O |
| ATOM | 3100 | OD2 | ASP | A | 449 | 6.619 | −94.419 | −47.444 | 1.00 | 63.29 | O |
| ATOM | 3101 | C | ASP | A | 449 | 3.868 | −92.743 | −51.014 | 1.00 | 62.46 | C |
| ATOM | 3102 | O | ASP | A | 449 | 2.684 | −92.959 | −50.717 | 1.00 | 62.44 | O |
| ATOM | 3103 | N | LYS | A | 450 | 4.253 | −91.756 | −51.817 | 1.00 | 62.30 | N |
| ATOM | 3104 | CA | LYS | A | 450 | 3.326 | −90.744 | −52.305 | 1.00 | 62.13 | C |
| ATOM | 3105 | CB | LYS | A | 450 | 4.051 | −89.734 | −53.196 | 1.00 | 62.11 | C |
| ATOM | 3106 | CG | LYS | A | 450 | 3.695 | −88.274 | −52.916 | 1.00 | 61.77 | C |
| ATOM | 3107 | CD | LYS | A | 450 | 2.292 | −87.897 | −53.378 | 1.00 | 60.83 | C |
| ATOM | 3108 | CE | LYS | A | 450 | 1.970 | −86.454 | −53.009 | 1.00 | 59.97 | C |
| ATOM | 3109 | NZ | LYS | A | 450 | 0.604 | −86.047 | −53.420 | 1.00 | 59.33 | N |
| ATOM | 3110 | C | LYS | A | 450 | 2.140 | −91.352 | −53.050 | 1.00 | 62.15 | C |
| ATOM | 3111 | O | LYS | A | 450 | 1.013 | −91.248 | −52.589 | 1.00 | 62.07 | O |
| ATOM | 3112 | N | SER | A | 451 | 2.394 | −91.983 | −54.193 | 1.00 | 62.16 | N |
| ATOM | 3113 | CA | SER | A | 451 | 1.320 | −92.559 | −55.008 | 1.00 | 62.17 | C |
| ATOM | 3114 | CB | SER | A | 451 | 1.913 | −93.360 | −56.164 | 1.00 | 62.12 | C |
| ATOM | 3115 | OG | SER | A | 451 | 3.202 | −93.834 | −55.821 | 1.00 | 62.06 | O |
| ATOM | 3116 | C | SER | A | 451 | 0.346 | −93.415 | −54.186 | 1.00 | 62.26 | C |
| ATOM | 3117 | O | SER | A | 451 | −0.870 | −93.404 | −54.419 | 1.00 | 62.26 | O |
| ATOM | 3118 | N | LYS | A | 452 | 0.895 | −94.144 | −53.221 | 1.00 | 62.25 | N |
| ATOM | 3119 | CA | LYS | A | 452 | 0.125 | −94.923 | −52.257 | 1.00 | 62.40 | C |
| ATOM | 3120 | CB | LYS | A | 452 | 1.131 | −95.657 | −51.364 | 1.00 | 62.38 | C |
| ATOM | 3121 | CG | LYS | A | 452 | 0.596 | −96.422 | −50.166 | 1.00 | 62.84 | C |
| ATOM | 3122 | CD | LYS | A | 452 | 1.727 | −97.330 | −49.630 | 1.00 | 63.07 | C |
| ATOM | 3123 | CE | LYS | A | 452 | 1.511 | −97.788 | −48.181 | 1.00 | 64.07 | C |
| ATOM | 3124 | NZ | LYS | A | 452 | 2.122 | −96.868 | −47.174 | 1.00 | 63.97 | N |
| ATOM | 3125 | C | LYS | A | 452 | −0.812 | −94.016 | −51.434 | 1.00 | 62.22 | C |
| ATOM | 3126 | O | LYS | A | 452 | −2.075 | −94.152 | −51.457 | 1.00 | 62.21 | O |
| ATOM | 3127 | N | TYR | A | 453 | −0.196 | −93.076 | −50.719 | 1.00 | 62.12 | N |
| ATOM | 3128 | CA | TYR | A | 453 | −0.965 | −92.110 | −49.949 | 1.00 | 61.83 | C |
| ATOM | 3129 | CB | TYR | A | 453 | −0.041 | −91.057 | −49.352 | 1.00 | 61.96 | C |
| ATOM | 3130 | CG | TYR | A | 453 | 0.911 | −91.611 | −48.324 | 1.00 | 62.18 | C |
| ATOM | 3131 | CD1 | TYR | A | 453 | 0.622 | −92.789 | −47.652 | 1.00 | 62.45 | C |
| ATOM | 3132 | CE1 | TYR | A | 453 | 1.484 | −93.298 | −46.704 | 1.00 | 62.52 | C |
| ATOM | 3133 | CZ | TYR | A | 453 | 2.645 | −92.622 | −46.408 | 1.00 | 62.28 | C |
| ATOM | 3134 | OH | TYR | A | 453 | 3.496 | −93.133 | −45.464 | 1.00 | 62.68 | O |
| ATOM | 3135 | CE2 | TYR | A | 453 | 2.957 | −91.445 | −47.053 | 1.00 | 62.18 | C |
| ATOM | 3136 | CD2 | TYR | A | 453 | 2.089 | −90.945 | −48.005 | 1.00 | 62.43 | C |
| ATOM | 3137 | C | TYR | A | 453 | −2.047 | −91.449 | −50.801 | 1.00 | 61.73 | C |
| ATOM | 3138 | O | TYR | A | 453 | −3.196 | −91.346 | −50.379 | 1.00 | 61.52 | O |
| ATOM | 3139 | N | ASP | A | 454 | −1.667 | −91.011 | −52.000 | 1.00 | 61.76 | N |
| ATOM | 3140 | CA | ASP | A | 454 | −2.583 | −90.401 | −52.962 | 1.00 | 61.68 | C |
| ATOM | 3141 | CB | ASP | A | 454 | −1.859 | −90.082 | −54.269 | 1.00 | 61.75 | C |
| ATOM | 3142 | CG | ASP | A | 454 | −1.090 | −88.786 | −54.208 | 1.00 | 62.05 | C |
| ATOM | 3143 | OD1 | ASP | A | 454 | −0.533 | −88.378 | −55.250 | 1.00 | 62.30 | O |
| ATOM | 3144 | OD2 | ASP | A | 454 | −1.045 | −88.176 | −53.119 | 1.00 | 63.12 | O |
| ATOM | 3145 | C | ASP | A | 454 | −3.761 | −91.304 | −53.267 | 1.00 | 61.60 | C |
| ATOM | 3146 | O | ASP | A | 454 | −4.908 | −90.860 | −53.236 | 1.00 | 61.46 | O |
| ATOM | 3147 | N | GLU | A | 455 | −3.474 | −92.565 | −53.581 | 1.00 | 61.64 | N |
| ATOM | 3148 | CA | GLU | A | 455 | −4.538 | −93.540 | −53.775 | 1.00 | 61.50 | C |
| ATOM | 3149 | CB | GLU | A | 455 | −3.972 | −94.962 | −53.839 | 1.00 | 61.52 | C |
| ATOM | 3150 | CG | GLU | A | 455 | −4.800 | −95.931 | −54.675 | 1.00 | 61.62 | C |
| ATOM | 3151 | CD | GLU | A | 455 | −3.526 | −96.015 | −55.853 | 0.00 | 50.00 | C |
| ATOM | 3152 | OE1 | GLU | A | 455 | −4.004 | −96.674 | −56.795 | 0.00 | 50.00 | O |
| ATOM | 3153 | OE2 | GLU | A | 455 | −2.549 | −95.268 | −55.967 | 0.00 | 50.00 | O |
| ATOM | 3154 | C | GLU | A | 455 | −5.543 | −93.399 | −52.630 | 1.00 | 61.47 | C |
| ATOM | 3155 | O | GLU | A | 455 | −6.770 | −93.244 | −52.874 | 1.00 | 61.39 | O |
| ATOM | 3156 | N | ILE | A | 456 | −5.034 | −93.395 | −51.379 | 1.00 | 61.57 | N |
| ATOM | 3157 | CA | ILE | A | 456 | −6.005 | −93.246 | −50.263 | 1.00 | 61.40 | C |
| ATOM | 3158 | CB | ILE | A | 456 | −5.373 | −93.420 | −48.849 | 1.00 | 61.41 | C |
| ATOM | 3159 | CG1 | ILE | A | 456 | −5.081 | −94.894 | −48.547 | 1.00 | 61.57 | C |
| ATOM | 3160 | CD1 | ILE | A | 456 | −3.780 | −95.411 | −49.117 | 1.00 | 61.87 | C |
| ATOM | 3161 | CG2 | ILE | A | 456 | −6.319 | −92.913 | −47.775 | 1.00 | 60.97 | C |
| ATOM | 3162 | C | ILE | A | 456 | −6.732 | −91.892 | −50.326 | 1.00 | 61.33 | C |
| ATOM | 3163 | O | ILE | A | 456 | −7.973 | −91.817 | −50.396 | 1.00 | 61.32 | O |
| ATOM | 3164 | N | PHE | A | 457 | −5.939 | −90.826 | −50.315 | 1.00 | 61.28 | N |
| ATOM | 3165 | CA | PHE | A | 457 | −6.451 | −89.462 | −50.304 | 1.00 | 61.18 | C |
| ATOM | 3166 | CB | PHE | A | 457 | −5.325 | −88.478 | −50.619 | 1.00 | 61.10 | C |
| ATOM | 3167 | CG | PHE | A | 457 | −5.788 | −87.059 | −50.805 | 1.00 | 61.06 | C |
| ATOM | 3168 | CD1 | PHE | A | 457 | −6.014 | −86.240 | −49.710 | 1.00 | 60.72 | C |
| ATOM | 3169 | CE1 | PHE | A | 457 | −6.425 | −84.930 | −49.878 | 1.00 | 60.37 | C |
| ATOM | 3170 | CZ | PHE | A | 457 | −6.609 | −84.424 | −51.146 | 1.00 | 60.51 | C |
| ATOM | 3171 | CE2 | PHE | A | 457 | −6.384 | −85.228 | −52.248 | 1.00 | 60.56 | C |
| ATOM | 3172 | CD2 | PHE | A | 457 | −5.976 | −86.536 | −52.077 | 1.00 | 60.85 | C |
| ATOM | 3173 | C | PHE | A | 457 | −7.583 | −89.275 | −51.295 | 1.00 | 61.08 | C |
| ATOM | 3174 | O | PHE | A | 457 | −8.642 | −88.746 | −50.955 | 1.00 | 61.04 | O |
| ATOM | 3175 | N | TYR | A | 458 | −7.352 | −89.710 | −52.526 | 1.00 | 61.12 | N |
| ATOM | 3176 | CA | TYR | A | 458 | −8.345 | −89.535 | −53.570 | 1.00 | 60.96 | C |
| ATOM | 3177 | CB | TYR | A | 458 | −7.707 | −89.605 | −54.954 | 1.00 | 60.87 | C |
| ATOM | 3178 | CG | TYR | A | 458 | −6.880 | −88.380 | −55.257 | 1.00 | 60.78 | C |
| ATOM | 3179 | CD1 | TYR | A | 458 | −7.477 | −87.135 | −55.376 | 1.00 | 60.62 | C |
| ATOM | 3180 | CE1 | TYR | A | 458 | −6.733 | −86.009 | −55.644 | 1.00 | 60.72 | C |
| ATOM | 3181 | CZ | TYR | A | 458 | −5.370 | −86.115 | −55.793 | 1.00 | 60.91 | C |
| ATOM | 3182 | OH | TYR | A | 458 | −4.624 | −84.987 | −56.061 | 1.00 | 61.14 | O |
| ATOM | 3183 | CE2 | TYR | A | 458 | −4.750 | −87.340 | −55.676 | 1.00 | 60.97 | C |
| ATOM | 3184 | CD2 | TYR | A | 458 | −5.505 | −88.463 | −55.404 | 1.00 | 60.82 | C |
| ATOM | 3185 | C | TYR | A | 458 | −9.497 | −90.509 | −53.423 | 1.00 | 60.98 | C |
| ATOM | 3186 | O | TYR | A | 458 | −10.613 | −90.212 | −53.844 | 1.00 | 60.99 | O |
| ATOM | 3187 | N | ASN | A | 459 | −9.246 | −91.663 | −52.807 | 1.00 | 61.12 | N |
| ATOM | 3188 | CA | ASN | A | 459 | −10.374 | −92.529 | −52.457 | 1.00 | 61.04 | C |
| ATOM | 3189 | CB | ASN | A | 459 | −9.935 | −93.967 | −52.193 | 1.00 | 61.02 | C |
| ATOM | 3190 | CG | ASN | A | 459 | −10.182 | −94.868 | −53.382 | 1.00 | 61.15 | C |
| ATOM | 3191 | OD1 | ASN | A | 459 | −9.353 | −95.713 | −53.720 | 1.00 | 61.72 | O |
| ATOM | 3192 | ND2 | ASN | A | 459 | −11.326 | −94.682 | −54.035 | 1.00 | 61.27 | N |
| ATOM | 3193 | C | ASN | A | 459 | −11.225 | −91.986 | −51.310 | 1.00 | 61.02 | C |
| ATOM | 3194 | O | ASN | A | 459 | −12.307 | −92.503 | −51.037 | 1.00 | 60.90 | O |
| ATOM | 3195 | N | LEU | A | 460 | −10.738 | −90.936 | −50.049 | 1.00 | 61.16 | N |
| ATOM | 3196 | CA | LEU | A | 460 | −11.536 | −90.256 | −49.614 | 1.00 | 61.21 | C |
| ATOM | 3197 | CB | LEU | A | 460 | −10.644 | −89.735 | −48.484 | 1.00 | 61.18 | C |
| ATOM | 3198 | CG | LEU | A | 460 | −10.441 | −90.651 | −47.276 | 1.00 | 61.31 | C |
| ATOM | 3199 | CD1 | LEU | A | 460 | −10.126 | −92.081 | −47.695 | 1.00 | 61.70 | C |
| ATOM | 3200 | CD2 | LEU | A | 460 | −9.353 | −90.103 | −46.364 | 1.00 | 61.36 | C |
| ATOM | 3201 | C | LEU | A | 460 | −12.427 | −89.118 | −50.132 | 1.00 | 61.21 | C |
| ATOM | 3202 | O | LEU | A | 460 | −13.042 | −88.401 | −49.341 | 1.00 | 61.15 | O |
| ATOM | 3203 | N | ALA | A | 461 | −12.490 | −88.958 | −51.452 | 1.00 | 61.30 | N |
| ATOM | 3204 | CA | ALA | A | 461 | −13.332 | −87.933 | −52.092 | 1.00 | 61.39 | C |
| ATOM | 3205 | CB | ALA | A | 461 | −14.810 | −88.328 | −52.035 | 1.00 | 61.32 | C |
| ATOM | 3206 | C | ALA | A | 461 | −13.132 | −86.526 | −51.527 | 1.00 | 61.46 | C |
| ATOM | 3207 | O | ALA | A | 461 | −13.989 | −86.016 | −50.803 | 1.00 | 61.46 | O |
| ATOM | 3208 | N | PRO | A | 462 | −11.998 | −85.891 | −51.868 | 1.00 | 61.52 | N |
| ATOM | 3209 | CA | PRO | A | 462 | −11.692 | −84.538 | −51.427 | 1.00 | 61.50 | C |
| ATOM | 3210 | CB | PRO | A | 462 | −10.172 | −84.439 | −51.605 | 1.00 | 61.48 | C |
| ATOM | 3211 | CG | PRO | A | 462 | −9.724 | −85.764 | −52.182 | 1.00 | 61.39 | C |
| ATOM | 3212 | CD | PRO | A | 462 | −10.929 | −86.437 | −52.716 | 1.00 | 61.48 | C |
| ATOM | 3213 | C | PRO | A | 462 | −12.388 | −83.514 | −52.310 | 1.00 | 61.55 | C |
| ATOM | 3214 | O | PRO | A | 462 | −12.933 | −83.873 | −53.351 | 1.00 | 61.39 | O |
| ATOM | 3215 | N | ALA | A | 463 | −12.359 | −82.252 | −51.891 | 1.00 | 61.78 | N |
| ATOM | 3216 | CA | ALA | A | 463 | −13.067 | −81.176 | −52.582 | 1.00 | 61.99 | C |
| ATOM | 3217 | CB | ALA | A | 463 | −12.888 | −79.860 | −51.840 | 1.00 | 62.09 | C |
| ATOM | 3218 | C | ALA | A | 463 | −12.611 | −81.031 | −54.023 | 1.00 | 62.16 | C |
| ATOM | 3219 | O | ALA | A | 463 | −13.233 | −81.565 | −54.938 | 1.00 | 62.42 | O |
| ATOM | 3220 | N | ASP | A | 464 | −11.528 | −80.293 | −54.226 | 1.00 | 62.26 | N |
| ATOM | 3221 | CA | ASP | A | 464 | −10.959 | −80.154 | −55.556 | 1.00 | 62.40 | C |
| ATOM | 3222 | CB | ASP | A | 464 | −11.255 | −78.767 | −56.133 | 1.00 | 62.60 | C |
| ATOM | 3223 | CG | ASP | A | 464 | −10.538 | −77.651 | −55.385 | 1.00 | 63.34 | C |
| ATOM | 3224 | OD1 | ASP | A | 464 | −10.634 | −76.485 | −55.835 | 1.00 | 63.76 | O |
| ATOM | 3225 | OD2 | ASP | A | 464 | −9.880 | −77.936 | −54.354 | 1.00 | 64.06 | O |
| ATOM | 3226 | C | ASP | A | 464 | −9.464 | −80.382 | −55.468 | 1.00 | 62.23 | C |
| ATOM | 3227 | O | ASP | A | 464 | −8.707 | −79.934 | −56.317 | 1.00 | 62.39 | O |
| ATOM | 3228 | N | GLY | A | 465 | −9.049 | −81.083 | −54.424 | 1.00 | 62.02 | N |
| ATOM | 3229 | CA | GLY | A | 465 | −7.640 | −81.324 | −54.177 | 1.00 | 61.82 | C |
| ATOM | 3230 | C | GLY | A | 465 | −7.398 | −81.127 | −52.700 | 1.00 | 61.70 | C |
| ATOM | 3231 | O | GLY | A | 465 | −6.284 | −81.310 | −52.205 | 1.00 | 61.73 | O |
| ATOM | 3232 | N | LYS | A | 466 | −8.460 | −80.752 | −51.996 | 1.00 | 61.43 | N |
| ATOM | 3233 | CA | LYS | A | 466 | −8.379 | −80.507 | −50.572 | 1.00 | 61.22 | C |
| ATOM | 3234 | CB | LYS | A | 466 | −8.571 | −79.023 | −50.279 | 1.00 | 61.21 | C |
| ATOM | 3235 | CG | LYS | A | 466 | −7.437 | −78.154 | −50.775 | 1.00 | 61.05 | C |
| ATOM | 3236 | CD | LYS | A | 466 | −7.845 | −76.698 | −50.831 | 1.00 | 61.32 | C |
| ATOM | 3237 | CE | LYS | A | 466 | −6.793 | −75.881 | −51.549 | 1.00 | 61.80 | C |
| ATOM | 3238 | NZ | LYS | A | 466 | −6.444 | −76.494 | −52.864 | 1.00 | 62.20 | N |
| ATOM | 3239 | C | LYS | A | 466 | −9.417 | −81.323 | −49.830 | 1.00 | 61.12 | C |
| ATOM | 3240 | O | LYS | A | 466 | −10.613 | −81.218 | −50.091 | 1.00 | 61.13 | O |
| ATOM | 3241 | N | LEU | A | 467 | −8.948 | −82.144 | −48.903 | 1.00 | 61.00 | N |
| ATOM | 3242 | CA | LEU | A | 467 | −9.841 | −82.926 | −48.074 | 1.00 | 60.88 | C |
| ATOM | 3243 | CB | LEU | A | 467 | −9.118 | −84.164 | −47.542 | 1.00 | 60.79 | C |
| ATOM | 3244 | CG | LEU | A | 467 | −9.961 | −85.111 | −46.693 | 1.00 | 60.87 | C |
| ATOM | 3245 | CD1 | LEU | A | 467 | −11.113 | −85.658 | −47.518 | 1.00 | 61.30 | C |
| ATOM | 3246 | CD2 | LEU | A | 467 | −9.114 | −86.240 | −46.140 | 1.00 | 60.91 | C |
| ATOM | 3247 | C | LEU | A | 467 | −10.339 | −82.065 | −46.917 | 1.00 | 60.83 | C |
| ATOM | 3248 | O | LEU | A | 467 | −9.544 | −81.491 | −46.171 | 1.00 | 60.86 | O |
| ATOM | 3249 | N | SER | A | 468 | −11.658 | −81.966 | −46.784 | 1.00 | 60.71 | N |
| ATOM | 3250 | CA | SER | A | 468 | −12.277 | −81.277 | −45.656 | 1.00 | 60.55 | C |
| ATOM | 3251 | CB | SER | A | 468 | −13.787 | −81.522 | −45.672 | 1.00 | 60.68 | C |
| ATOM | 3252 | OG | SER | A | 468 | −14.306 | −81.658 | −44.359 | 1.00 | 61.03 | O |
| ATOM | 3253 | C | SER | A | 468 | −11.682 | −81.727 | −44.319 | 1.00 | 60.34 | C |
| ATOM | 3254 | O | SER | A | 468 | −11.179 | −82.846 | −44.201 | 1.00 | 60.29 | O |
| ATOM | 3255 | N | GLY | A | 469 | −11.746 | −80.852 | −43.318 | 1.00 | 60.13 | N |
| ATOM | 3256 | CA | GLY | A | 469 | −11.213 | −81.153 | −41.989 | 1.00 | 59.92 | C |
| ATOM | 3257 | C | GLY | A | 469 | −11.948 | −82.273 | −41.272 | 1.00 | 59.86 | C |
| ATOM | 3258 | O | GLY | A | 469 | −11.335 | −83.108 | −40.605 | 1.00 | 59.75 | O |
| ATOM | 3259 | N | SER | A | 470 | −13.267 | −82.298 | −41.412 | 1.00 | 59.92 | N |
| ATOM | 3260 | CA | SER | A | 470 | −14.080 | −83.316 | −40.755 | 1.00 | 60.13 | C |
| ATOM | 3261 | CB | SER | A | 470 | −15.570 | −82.996 | −40.911 | 1.00 | 60.18 | C |
| ATOM | 3262 | OG | SER | A | 470 | −15.909 | −82.804 | −42.273 | 1.00 | 60.39 | O |
| ATOM | 3263 | C | SER | A | 470 | −13.771 | −84.730 | −41.262 | 1.00 | 60.16 | C |
| ATOM | 3264 | O | SER | A | 470 | −13.628 | −85.667 | −40.468 | 1.00 | 60.19 | O |
| ATOM | 3265 | N | LYS | A | 471 | −13.665 | −84.875 | −42.582 | 1.00 | 60.16 | N |
| ATOM | 3266 | CA | LYS | A | 471 | −13.353 | −86.160 | −43.203 | 1.00 | 60.23 | C |
| ATOM | 3267 | CB | LYS | A | 471 | −13.418 | −86.032 | −44.726 | 1.00 | 60.32 | C |
| ATOM | 3268 | CG | LYS | A | 471 | −14.215 | −84.826 | −45.209 | 1.00 | 60.75 | C |
| ATOM | 3269 | CD | LYS | A | 471 | −15.690 | −85.140 | −45.412 | 1.00 | 61.90 | C |
| ATOM | 3270 | CE | LYS | A | 471 | −16.083 | −85.072 | −46.889 | 1.00 | 62.37 | C |
| ATOM | 3271 | NZ | LYS | A | 471 | −15.249 | −85.959 | −47.753 | 1.00 | 62.88 | N |
| ATOM | 3272 | C | LYS | A | 471 | −11.952 | −86.600 | −42.777 | 1.00 | 60.16 | C |
| ATOM | 3273 | O | LYS | A | 471 | −11.723 | −87.758 | −42.368 | 1.00 | 60.14 | O |
| ATOM | 3274 | N | ALA | A | 472 | −11.021 | −85.654 | −42.869 | 1.00 | 60.13 | N |
| ATOM | 3275 | CA | ALA | A | 472 | −9.655 | −85.868 | −42.425 | 1.00 | 60.03 | C |
| ATOM | 3276 | CB | ALA | A | 472 | −8.853 | −84.582 | −42.529 | 1.00 | 59.90 | C |
| ATOM | 3277 | C | ALA | A | 472 | −9.670 | −86.382 | −40.993 | 1.00 | 59.97 | C |
| ATOM | 3278 | O | ALA | A | 472 | −8.988 | −87.358 | −40.665 | 1.00 | 60.05 | O |
| ATOM | 3279 | N | LYS | A | 473 | −10.466 | −85.741 | −40.144 | 1.00 | 59.85 | N |
| ATOM | 3280 | CA | LYS | A | 473 | −10.563 | −86.203 | −38.771 | 1.00 | 59.96 | C |
| ATOM | 3281 | CB | LYS | A | 473 | −11.393 | −85.269 | −37.895 | 1.00 | 59.91 | C |
| ATOM | 3282 | CG | LYS | A | 473 | −11.375 | −85.683 | −36.428 | 1.00 | 59.67 | C |
| ATOM | 3283 | CD | LYS | A | 473 | −12.269 | −84.800 | −35.574 | 1.00 | 59.75 | C |
| ATOM | 3284 | CE | LYS | A | 473 | −11.937 | −84.943 | −34.095 | 1.00 | 59.64 | C |
| ATOM | 3285 | NZ | LYS | A | 473 | −12.834 | −84.122 | −33.234 | 1.00 | 59.71 | N |
| ATOM | 3286 | C | LYS | A | 473 | −11.111 | −87.620 | −38.700 | 1.00 | 60.14 | C |
| ATOM | 3287 | O | LYS | A | 473 | −10.441 | −88.495 | −38.164 | 1.00 | 60.26 | O |
| ATOM | 3288 | N | THR | A | 474 | −12.306 | −87.866 | −39.243 | 1.00 | 60.16 | N |
| ATOM | 3289 | CA | THR | A | 474 | −12.872 | −89.219 | −39.144 | 1.00 | 60.24 | C |
| ATOM | 3290 | CB | THR | A | 474 | −14.257 | −89.402 | −39.852 | 1.00 | 60.23 | C |
| ATOM | 3291 | OG1 | THR | A | 474 | −14.300 | −88.662 | −41.075 | 1.00 | 60.62 | O |
| ATOM | 3292 | CG2 | THR | A | 474 | −15.394 | −88.933 | −38.952 | 1.00 | 60.37 | C |
| ATOM | 3293 | C | THR | A | 474 | −11.864 | −90.289 | −39.582 | 1.00 | 60.23 | C |
| ATOM | 3294 | O | THR | A | 474 | −11.719 | −91.319 | −38.917 | 1.00 | 60.25 | O |
| ATOM | 3295 | N | TRP | A | 475 | −11.145 | −90.037 | −40.672 | 1.00 | 60.28 | N |
| ATOM | 3296 | CA | TRP | A | 475 | −10.106 | −90.978 | −41.096 | 1.00 | 60.42 | C |
| ATOM | 3297 | CB | TRP | A | 475 | −9.523 | −90.535 | −42.438 | 1.00 | 60.84 | C |
| ATOM | 3298 | CG | TRP | A | 475 | −8.313 | −91.301 | −42.870 | 1.00 | 61.18 | C |
| ATOM | 3299 | CD1 | TRP | A | 475 | −8.275 | −92.585 | −43.318 | 1.00 | 61.70 | C |
| ATOM | 3300 | NE1 | TRP | A | 475 | −6.985 | −92.942 | −43.627 | 1.00 | 61.88 | N |
| ATOM | 3301 | CE2 | TRP | A | 475 | −6.160 | −91.878 | −43.383 | 1.00 | 61.50 | C |
| ATOM | 3302 | CD2 | TRP | A | 475 | −6.963 | −90.823 | −42.907 | 1.00 | 61.57 | C |
| ATOM | 3303 | CE3 | TRP | A | 475 | −6.354 | −89.606 | −42.577 | 1.00 | 62.24 | C |
| ATOM | 3304 | CZ3 | TRP | A | 475 | −4.977 | −89.484 | −42.734 | 1.00 | 62.05 | C |
| ATOM | 3305 | CH2 | TRP | A | 475 | −4.207 | −90.555 | −43.212 | 1.00 | 61.82 | C |
| ATOM | 3306 | CZ2 | TRP | A | 475 | −4.780 | −91.756 | −43.540 | 1.00 | 61.71 | C |
| ATOM | 3307 | C | TRP | A | 475 | −8.998 | −91.098 | −40.043 | 1.00 | 60.37 | C |
| ATOM | 3308 | O | TRP | A | 475 | −8.652 | −92.206 | −39.568 | 1.00 | 60.28 | O |
| ATOM | 3309 | N | MET | A | 476 | −8.444 | −89.948 | −39.674 | 1.00 | 60.24 | N |
| ATOM | 3310 | CA | MET | A | 476 | −7.358 | −89.927 | −38.717 | 1.00 | 60.43 | C |
| ATOM | 3311 | CB | MET | A | 476 | −6.936 | −88.492 | −38.400 | 1.00 | 60.35 | C |
| ATOM | 3312 | CG | MET | A | 476 | −6.136 | −87.837 | −39.519 | 1.00 | 60.65 | C |
| ATOM | 3313 | SD | MET | A | 476 | −5.609 | −86.145 | −39.154 | 1.00 | 60.97 | S |
| ATOM | 3314 | CE | MET | A | 476 | −4.147 | −86.452 | −38.158 | 1.00 | 61.19 | C |
| ATOM | 3315 | C | MET | A | 476 | −7.739 | −90.692 | −37.452 | 1.00 | 60.37 | C |
| ATOM | 3316 | O | MET | A | 476 | −7.049 | −91.632 | −37.055 | 1.00 | 60.37 | O |
| ATOM | 3317 | N | VAL | A | 477 | −8.847 | −90.304 | −36.830 | 1.00 | 60.31 | N |
| ATOM | 3318 | CA | VAL | A | 477 | −9.308 | −90.980 | −35.630 | 1.00 | 60.18 | C |
| ATOM | 3319 | CB | VAL | A | 477 | −10.572 | −90.317 | −35.039 | 1.00 | 60.16 | C |
| ATOM | 3320 | CG1 | VAL | A | 477 | −11.834 | −91.039 | −35.497 | 1.00 | 60.18 | C |
| ATOM | 3321 | CG2 | VAL | A | 477 | −10.491 | −90.304 | −33.519 | 1.00 | 60.61 | C |
| ATOM | 3322 | C | VAL | A | 477 | −9.553 | −92.442 | −35.979 | 1.00 | 60.04 | C |
| ATOM | 3323 | O | VAL | A | 477 | −9.463 | −93.322 | −35.124 | 1.00 | 59.92 | O |
| ATOM | 3324 | N | GLY | A | 478 | −9.836 | −92.689 | −37.255 | 1.00 | 60.05 | N |
| ATOM | 3325 | CA | GLY | A | 478 | −9.937 | −94.048 | −37.773 | 1.00 | 59.87 | C |
| ATOM | 3326 | C | GLY | A | 478 | −8.652 | −94.826 | −37.573 | 1.00 | 59.75 | C |
| ATOM | 3327 | O | GLY | A | 478 | −8.683 | −96.039 | −37.375 | 1.00 | 59.64 | O |
| ATOM | 3328 | N | THR | A | 479 | −7.512 | −94.139 | −37.618 | 1.00 | 59.79 | N |
| ATOM | 3329 | CA | THR | A | 479 | −6.230 | −94.827 | −37.337 | 1.00 | 59.54 | C |
| ATOM | 3330 | CB | THR | A | 479 | −5.012 | −93.965 | −37.682 | 1.00 | 59.50 | C |
| ATOM | 3331 | OG1 | THR | A | 479 | −4.708 | −93.109 | −36.572 | 1.00 | 59.40 | O |
| ATOM | 3332 | CG2 | THR | A | 479 | −5.274 | −93.142 | −38.930 | 1.00 | 59.72 | C |
| ATOM | 3333 | C | THR | A | 479 | −6.044 | −95.280 | −35.881 | 1.00 | 59.41 | C |
| ATOM | 3334 | O | THR | A | 479 | −4.929 | −95.606 | −35.467 | 1.00 | 59.36 | O |
| ATOM | 3335 | N | LYS | A | 480 | −7.126 | −95.279 | −35.111 | 1.00 | 59.24 | N |
| ATOM | 3336 | CA | LYS | A | 480 | −7.102 | −95.706 | −33.708 | 1.00 | 59.00 | C |
| ATOM | 3337 | CB | LYS | A | 480 | −7.107 | −97.236 | −33.602 | 1.00 | 58.90 | C |
| ATOM | 3338 | CG | LYS | A | 480 | −8.720 | −97.582 | −33.472 | 0.00 | 50.00 | C |
| ATOM | 3339 | CD | LYS | A | 480 | −8.884 | −99.002 | −33.964 | 0.00 | 50.00 | C |
| ATOM | 3340 | CE | LYS | A | 480 | −7.623 | −99.825 | −33.831 | 0.00 | 50.00 | C |
| ATOM | 3341 | NZ | LYS | A | 480 | −7.163 | −100.001 | −32.431 | 0.00 | 50.00 | N |
| ATOM | 3342 | C | LYS | A | 480 | −5.952 | −95.120 | −32.883 | 1.00 | 58.86 | C |
| ATOM | 3343 | O | LYS | A | 480 | −5.432 | −95.778 | −31.985 | 1.00 | 59.02 | O |
| ATOM | 3344 | N | LEU | A | 481 | −5.562 | −93.884 | −33.184 | 1.00 | 58.63 | N |
| ATOM | 3345 | CA | LEU | A | 481 | −4.589 | −93.169 | −32.361 | 1.00 | 58.40 | C |
| ATOM | 3346 | CB | LEU | A | 481 | −3.644 | −92.336 | −33.233 | 1.00 | 58.41 | C |
| ATOM | 3347 | CG | LEU | A | 481 | −2.448 | −93.045 | −33.870 | 1.00 | 58.17 | C |
| ATOM | 3348 | CD1 | LEU | A | 481 | −1.516 | −92.033 | −34.503 | 1.00 | 57.72 | C |
| ATOM | 3349 | CD2 | LEU | A | 481 | −1.700 | −93.871 | −32.840 | 1.00 | 57.96 | C |
| ATOM | 3350 | C | LEU | A | 481 | −5.303 | −92.264 | −31.359 | 1.00 | 58.27 | C |
| ATOM | 3351 | O | LEU | A | 481 | −6.429 | −91.835 | −31.607 | 1.00 | 58.35 | O |
| ATOM | 3352 | N | PRO | A | 482 | −4.656 | −91.980 | −30.216 | 1.00 | 58.15 | N |
| ATOM | 3353 | CA | PRO | A | 482 | −5.239 | −91.080 | −29.221 | 1.00 | 58.10 | C |
| ATOM | 3354 | CB | PRO | A | 482 | −4.232 | −91.119 | −28.068 | 1.00 | 58.03 | C |
| ATOM | 3355 | CG | PRO | A | 482 | −2.965 | −91.591 | −28.671 | 1.00 | 58.03 | C |
| ATOM | 3356 | CD | PRO | A | 482 | −3.349 | −92.507 | −29.787 | 1.00 | 58.12 | C |
| ATOM | 3357 | C | PRO | A | 482 | −5.389 | −89.658 | −29.749 | 1.00 | 58.13 | C |
| ATOM | 3358 | O | PRO | A | 482 | −4.523 | −89.170 | −30.476 | 1.00 | 58.05 | O |
| ATOM | 3359 | N | ASN | A | 483 | −6.490 | −89.010 | −29.375 | 1.00 | 58.25 | N |
| ATOM | 3360 | CA | ASN | A | 483 | −6.826 | −87.673 | −29.865 | 1.00 | 58.27 | C |
| ATOM | 3361 | CB | ASN | A | 483 | −7.966 | −87.069 | −29.037 | 1.00 | 58.41 | C |
| ATOM | 3362 | CG | ASN | A | 483 | −8.680 | −85.936 | −29.761 | 1.00 | 58.98 | C |
| ATOM | 3363 | OD1 | ASN | A | 483 | −8.648 | −84.782 | −29.323 | 1.00 | 58.96 | O |
| ATOM | 3364 | ND2 | ASN | A | 483 | −9.328 | −86.264 | −30.878 | 1.00 | 59.69 | N |
| ATOM | 3365 | C | ASN | A | 483 | −5.630 | −86.730 | −29.872 | 1.00 | 57.99 | C |
| ATOM | 3366 | O | ASN | A | 483 | −5.249 | −86.219 | −30.912 | 1.00 | 57.84 | O |
| ATOM | 3367 | N | SER | A | 484 | −5.048 | −86.515 | −28.698 | 1.00 | 57.91 | N |
| ATOM | 3368 | CA | SER | A | 484 | −3.895 | −85.634 | −28.518 | 1.00 | 57.86 | C |
| ATOM | 3369 | CB | SER | A | 484 | −3.279 | −85.891 | −27.137 | 1.00 | 57.83 | C |
| ATOM | 3370 | OG | SER | A | 484 | −1.867 | −85.789 | −27.157 | 1.00 | 58.63 | O |
| ATOM | 3371 | C | SER | A | 484 | −2.851 | −85.771 | −29.634 | 1.00 | 57.69 | C |
| ATOM | 3372 | O | SER | A | 484 | −2.538 | −84.801 | −30.341 | 1.00 | 57.89 | O |
| ATOM | 3373 | N | VAL | A | 485 | −2.323 | −86.978 | −29.798 | 1.00 | 57.50 | N |
| ATOM | 3374 | CA | VAL | A | 485 | −1.368 | −87.246 | −30.858 | 1.00 | 57.27 | C |
| ATOM | 3375 | CB | VAL | A | 485 | −1.034 | −88.746 | −30.950 | 1.00 | 57.16 | C |
| ATOM | 3376 | CG1 | VAL | A | 485 | −0.287 | −89.050 | −32.233 | 1.00 | 56.97 | C |
| ATOM | 3377 | CG2 | VAL | A | 485 | −0.213 | −89.173 | −29.750 | 1.00 | 56.96 | C |
| ATOM | 3378 | C | VAL | A | 485 | −1.925 | −86.756 | −32.186 | 1.00 | 57.35 | C |
| ATOM | 3379 | O | VAL | A | 485 | −1.303 | −85.944 | −32.866 | 1.00 | 57.43 | O |
| ATOM | 3380 | N | LEU | A | 486 | −3.113 | −87.233 | −32.542 | 1.00 | 57.54 | N |
| ATOM | 3381 | CA | LEU | A | 486 | −3.739 | −86.843 | −33.803 | 1.00 | 57.77 | C |
| ATOM | 3382 | CB | LEU | A | 486 | −5.138 | −87.454 | −33.937 | 1.00 | 57.88 | C |
| ATOM | 3383 | CG | LEU | A | 486 | −5.229 | −88.972 | −34.109 | 1.00 | 58.42 | C |
| ATOM | 3384 | CD1 | LEU | A | 486 | −6.672 | −89.447 | −33.979 | 1.00 | 58.66 | C |
| ATOM | 3385 | CD2 | LEU | A | 486 | −4.638 | −89.407 | −35.444 | 1.00 | 58.90 | C |
| ATOM | 3386 | C | LEU | A | 486 | −3.803 | −85.325 | −33.975 | 1.00 | 57.80 | C |
| ATOM | 3387 | O | LEU | A | 486 | −3.455 | −84.808 | −35.033 | 1.00 | 57.86 | O |
| ATOM | 3388 | N | GLY | A | 487 | −4.249 | −84.618 | −32.938 | 1.00 | 57.71 | N |
| ATOM | 3389 | CA | GLY | A | 487 | −4.357 | −83.163 | −32.979 | 1.00 | 57.64 | C |
| ATOM | 3390 | C | GLY | A | 487 | −3.016 | −82.531 | −33.282 | 1.00 | 57.70 | C |
| ATOM | 3391 | O | GLY | A | 487 | −2.906 | −81.638 | −34.142 | 1.00 | 57.71 | O |
| ATOM | 3392 | N | ARG | A | 488 | −1.988 | −83.010 | −32.585 | 1.00 | 57.78 | N |
| ATOM | 3393 | CA | ARG | A | 488 | −0.628 | −82.544 | −32.841 | 1.00 | 57.97 | C |
| ATOM | 3394 | CB | ARG | A | 488 | 0.348 | −83.209 | −31.870 | 1.00 | 58.06 | C |
| ATOM | 3395 | CG | ARG | A | 488 | 1.529 | −82.338 | −31.468 | 1.00 | 59.39 | C |
| ATOM | 3396 | CD | ARG | A | 488 | 1.890 | −82.573 | −30.002 | 1.00 | 61.62 | C |
| ATOM | 3397 | NE | ARG | A | 488 | 2.408 | −83.923 | −29.761 | 1.00 | 63.12 | N |
| ATOM | 3398 | CZ | ARG | A | 488 | 2.051 | −84.695 | −28.733 | 1.00 | 63.38 | C |
| ATOM | 3399 | NH1 | ARG | A | 488 | 2.580 | −85.907 | −28.598 | 1.00 | 63.40 | N |
| ATOM | 3400 | NH2 | ARG | A | 488 | 1.153 | −84.266 | −27.850 | 1.00 | 63.17 | N |
| ATOM | 3401 | C | ARG | A | 488 | −0.227 | −82.814 | −34.300 | 1.00 | 57.81 | C |
| ATOM | 3402 | O | ARG | A | 488 | 0.355 | −81.947 | −34.979 | 1.00 | 57.77 | O |
| ATOM | 3403 | N | ILE | A | 489 | −0.564 | −84.008 | −34.784 | 1.00 | 57.58 | N |
| ATOM | 3404 | CA | ILE | A | 489 | −0.257 | −84.377 | −36.159 | 1.00 | 57.48 | C |
| ATOM | 3405 | CB | ILE | A | 489 | −0.663 | −85.815 | −36.470 | 1.00 | 57.33 | C |
| ATOM | 3406 | CG1 | ILE | A | 489 | 0.244 | −86.784 | −35.716 | 1.00 | 57.24 | C |
| ATOM | 3407 | CD1 | ILE | A | 489 | −0.047 | −88.239 | −35.980 | 1.00 | 57.50 | C |
| ATOM | 3408 | CG2 | ILE | A | 489 | −0.568 | −86.066 | −37.958 | 1.00 | 57.45 | C |
| ATOM | 3409 | C | ILE | A | 489 | −0.953 | −83.441 | −37.128 | 1.00 | 57.56 | C |
| ATOM | 3410 | O | ILE | A | 489 | −0.344 | −82.935 | −38.070 | 1.00 | 57.63 | O |
| ATOM | 3411 | N | TRP | A | 490 | −2.236 | −83.206 | −36.895 | 1.00 | 57.67 | N |
| ATOM | 3412 | CA | TRP | A | 490 | −2.970 | −82.265 | −37.711 | 1.00 | 57.64 | C |
| ATOM | 3413 | CB | TRP | A | 490 | −4.367 | −82.008 | −37.152 | 1.00 | 57.40 | C |
| ATOM | 3414 | CG | TRP | A | 490 | −4.998 | −80.874 | −37.862 | 1.00 | 57.22 | C |
| ATOM | 3415 | CD1 | TRP | A | 490 | −4.966 | −79.563 | −37.500 | 1.00 | 56.97 | C |
| ATOM | 3416 | NE1 | TRP | A | 490 | −5.628 | −78.800 | −38.430 | 1.00 | 57.04 | N |
| ATOM | 3417 | CE2 | TRP | A | 490 | −6.092 | −79.619 | −39.425 | 1.00 | 57.10 | C |
| ATOM | 3418 | CD2 | TRP | A | 490 | −5.704 | −80.933 | −39.103 | 1.00 | 57.28 | C |
| ATOM | 3419 | CE3 | TRP | A | 490 | −6.056 | −81.974 | −39.970 | 1.00 | 57.40 | C |
| ATOM | 3420 | CZ3 | TRP | A | 490 | −6.780 | −81.672 | −41.109 | 1.00 | 57.15 | C |
| ATOM | 3421 | CH2 | TRP | A | 490 | −7.154 | −80.354 | −41.400 | 1.00 | 57.10 | C |
| ATOM | 3422 | CZ2 | TRP | A | 490 | −6.820 | −79.316 | −40.573 | 1.00 | 57.08 | C |
| ATOM | 3423 | C | TRP | A | 490 | −2.208 | −80.950 | −37.754 | 1.00 | 57.62 | C |
| ATOM | 3424 | O | TRP | A | 490 | −1.865 | −80.442 | −38.833 | 1.00 | 57.65 | O |
| ATOM | 3425 | N | LYS | A | 491 | −1.942 | −80.409 | −36.568 | 1.00 | 51.68 | N |
| ATOM | 3426 | CA | LYS | A | 491 | −1.302 | −79.103 | −36.485 | 1.00 | 57.74 | C |
| ATOM | 3427 | CB | LYS | A | 491 | −1.059 | −78.684 | −35.036 | 1.00 | 57.75 | C |
| ATOM | 3428 | CG | LYS | A | 491 | −0.483 | −77.286 | −34.909 | 1.00 | 58.40 | C |
| ATOM | 3429 | CD | LYS | A | 491 | −0.964 | −76.592 | −33.647 | 1.00 | 60.09 | C |
| ATOM | 3430 | CE | LYS | A | 491 | −2.402 | −76.107 | −33.804 | 1.00 | 61.13 | C |
| ATOM | 3431 | NZ | LYS | A | 491 | −2.486 | −75.022 | −34.833 | 1.00 | 62.38 | N |
| ATOM | 3432 | C | LYS | A | 491 | −0.009 | −79.077 | −37.281 | 1.00 | 57.58 | C |
| ATOM | 3433 | O | LYS | A | 491 | 0.285 | −78.090 | −37.958 | 1.00 | 57.50 | O |
| ATOM | 3434 | N | LEU | A | 492 | 0.754 | −80.166 | −37.203 | 1.00 | 57.51 | N |
| ATOM | 3435 | CA | LEU | A | 492 | 1.982 | −80.287 | −37.988 | 1.00 | 57.34 | C |
| ATOM | 3436 | CB | LEU | A | 492 | 2.773 | −81.513 | −37.538 | 1.00 | 57.12 | C |
| ATOM | 3437 | CG | LEU | A | 492 | 4.058 | −81.329 | −36.735 | 1.00 | 56.50 | C |
| ATOM | 3438 | CD1 | LEU | A | 492 | 4.013 | −80.090 | −35.863 | 1.00 | 56.61 | C |
| ATOM | 3439 | CD2 | LEU | A | 492 | 4.317 | −82.581 | −35.908 | 1.00 | 56.14 | C |
| ATOM | 3440 | C | LEU | A | 492 | 1.722 | −80.363 | −39.500 | 1.00 | 57.59 | C |
| ATOM | 3441 | O | LEU | A | 492 | 2.471 | −79.801 | −40.301 | 1.00 | 57.53 | O |
| ATOM | 3442 | N | SER | A | 493 | 0.653 | −81.051 | −39.885 | 1.00 | 57.77 | N |
| ATOM | 3443 | CA | SER | A | 493 | 0.421 | −81.357 | −41.288 | 1.00 | 58.08 | C |
| ATOM | 3444 | CB | SER | A | 493 | −0.393 | −82.641 | −41.427 | 1.00 | 58.12 | C |
| ATOM | 3445 | OG | SER | A | 493 | 0.344 | −83.746 | −40.936 | 1.00 | 58.62 | O |
| ATOM | 3446 | C | SER | A | 493 | −0.238 | −80.231 | −42.071 | 1.00 | 58.20 | C |
| ATOM | 3447 | O | SER | A | 493 | 0.068 | −80.036 | −43.249 | 1.00 | 58.25 | O |
| ATOM | 3448 | N | ASP | A | 494 | −1.150 | −79.499 | −41.440 | 1.00 | 58.27 | N |
| ATOM | 3449 | CA | ASP | A | 494 | −1.797 | −78.397 | −42.148 | 1.00 | 58.62 | C |
| ATOM | 3450 | CB | ASP | A | 494 | −3.062 | −77.935 | −41.434 | 1.00 | 58.56 | C |
| ATOM | 3451 | CG | ASP | A | 494 | −3.824 | −76.894 | −42.226 | 1.00 | 58.70 | C |
| ATOM | 3452 | OD1 | ASP | A | 494 | −3.470 | −76.661 | −43.405 | 1.00 | 58.46 | O |
| ATOM | 3453 | OD2 | ASP | A | 494 | −4.777 | −76.308 | −41.668 | 1.00 | 59.37 | O |
| ATOM | 3454 | C | ASP | A | 494 | −0.821 | −77.238 | −42.302 | 1.00 | 58.87 | C |
| ATOM | 3455 | O | ASP | A | 494 | −0.883 | −76.250 | −41.568 | 1.00 | 58.88 | O |
| ATOM | 3456 | N | VAL | A | 495 | 0.078 | −77.368 | −43.269 | 1.00 | 59.15 | N |
| ATOM | 3457 | CA | VAL | A | 495 | 1.182 | −76.438 | −43.406 | 1.00 | 59.38 | C |
| ATOM | 3458 | CB | VAL | A | 495 | 2.219 | −76.921 | −44.439 | 1.00 | 59.37 | C |
| ATOM | 3459 | CG1 | VAL | A | 495 | 3.314 | −75.883 | −44.618 | 1.00 | 59.25 | C |
| ATOM | 3460 | CG2 | VAL | A | 495 | 2.819 | −78.234 | −43.989 | 1.00 | 59.21 | C |
| ATOM | 3461 | C | VAL | A | 495 | 0.721 | −75.025 | −43.728 | 1.00 | 59.65 | C |
| ATOM | 3462 | O | VAL | A | 495 | 1.173 | −74.077 | −43.089 | 1.00 | 60.04 | O |
| ATOM | 3463 | N | ASP | A | 496 | −0.171 | −74.869 | −44.703 | 1.00 | 59.85 | N |
| ATOM | 3464 | CA | ASP | A | 496 | −0.668 | −73.525 | −45.031 | 1.00 | 60.04 | C |
| ATOM | 3465 | CB | ASP | A | 496 | −1.133 | −73.422 | −46.488 | 1.00 | 60.09 | C |
| ATOM | 3466 | CG | ASP | A | 496 | −2.176 | −74.455 | −46.847 | 1.00 | 60.31 | C |
| ATOM | 3467 | OD1 | ASP | A | 496 | −2.344 | −75.432 | −46.082 | 1.00 | 60.71 | O |
| ATOM | 3468 | OD2 | ASP | A | 496 | −2.825 | −74.289 | −47.904 | 1.00 | 60.45 | O |
| ATOM | 3469 | C | ASP | A | 496 | −1.771 | −73.111 | −44.066 | 1.00 | 60.06 | C |
| ATOM | 3470 | O | ASP | A | 496 | −2.373 | −72.046 | −44.210 | 1.00 | 59.85 | O |
| ATOM | 3471 | N | ARG | A | 497 | −1.999 | −73.980 | −43.081 | 1.00 | 60.35 | N |
| ATOM | 3472 | CA | ARG | A | 497 | −2.962 | −73.785 | −41.992 | 1.00 | 60.65 | C |
| ATOM | 3473 | CB | ARG | A | 497 | −2.285 | −73.166 | −40.754 | 1.00 | 60.76 | C |
| ATOM | 3474 | CG | ARG | A | 497 | −2.301 | −71.656 | −40.649 | 1.00 | 62.10 | C |
| ATOM | 3475 | CD | ARG | A | 497 | −1.067 | −71.162 | −39.908 | 1.00 | 64.37 | C |
| ATOM | 3476 | NE | ARG | A | 497 | 0.050 | −70.975 | −40.832 | 1.00 | 66.05 | N |
| ATOM | 3477 | CZ | ARG | A | 497 | 0.345 | −69.817 | −41.420 | 1.00 | 67.08 | C |
| ATOM | 3478 | NH1 | ARG | A | 497 | 1.375 | −69.738 | −42.255 | 1.00 | 67.52 | N |
| ATOM | 3479 | NH2 | ARG | A | 497 | −0.385 | −68.733 | −41.170 | 1.00 | 67.06 | N |
| ATOM | 3480 | C | ARG | A | 497 | −4.271 | −73.097 | −42.402 | 1.00 | 60.58 | C |
| ATOM | 3481 | O | ARG | A | 497 | −4.638 | −72.036 | −41.889 | 1.00 | 60.38 | O |
| ATOM | 3482 | N | ASP | A | 498 | −4.976 | −73.740 | −43.331 | 1.00 | 60.75 | N |
| ATOM | 3483 | CA | ASP | A | 498 | −6.248 | −73.227 | −43.821 | 1.00 | 60.92 | C |
| ATOM | 3484 | CB | ASP | A | 498 | −6.279 | −73.207 | −45.358 | 1.00 | 60.94 | C |
| ATOM | 3485 | CG | ASP | A | 498 | −6.136 | −74.597 | −45.961 | 1.00 | 60.96 | C |
| ATOM | 3486 | OD1 | ASP | A | 498 | −5.577 | −75.493 | −45.290 | 1.00 | 61.07 | O |
| ATOM | 3487 | OD2 | ASP | A | 498 | −6.578 | −74.792 | −47.115 | 1.00 | 60.82 | O |
| ATOM | 3488 | C | ASP | A | 498 | −7.435 | −74.025 | −43.273 | 1.00 | 61.06 | C |
| ATOM | 3489 | O | ASP | A | 498 | −8.584 | −73.496 | −43.189 | 1.00 | 61.39 | O |
| ATOM | 3490 | N | GLY | A | 499 | −7.172 | −75.293 | −42.902 | 1.00 | 61.04 | N |
| ATOM | 3491 | CA | GLY | A | 499 | −8.246 | −76.100 | −42.330 | 1.00 | 61.21 | C |
| ATOM | 3492 | C | GLY | A | 499 | −8.543 | −77.366 | −43.108 | 1.00 | 61.37 | C |
| ATOM | 3493 | O | GLY | A | 499 | −9.301 | −78.229 | −42.648 | 1.00 | 61.52 | O |
| ATOM | 3494 | N | MET | A | 500 | −7.948 | −77.476 | −44.291 | 1.00 | 61.28 | N |
| ATOM | 3495 | CA | MET | A | 500 | −8.092 | −78.669 | −45.105 | 1.00 | 61.23 | C |
| ATOM | 3496 | CB | MET | A | 500 | −8.669 | −78.304 | −46.467 | 1.00 | 61.21 | C |
| ATOM | 3497 | CG | MET | A | 500 | −10.072 | −77.741 | −46.410 | 1.00 | 61.10 | C |
| ATOM | 3498 | SD | MET | A | 500 | −10.597 | −77.079 | −48.004 | 1.00 | 61.13 | S |
| ATOM | 3499 | CE | MET | A | 500 | −12.361 | −76.890 | −47.731 | 1.00 | 61.68 | C |
| ATOM | 3500 | C | MET | A | 500 | −6.737 | −79.338 | −45.277 | 1.00 | 61.33 | C |
| ATOM | 3501 | O | MET | A | 500 | −5.721 | −78.816 | −44.820 | 1.00 | 61.29 | O |
| ATOM | 3502 | N | LEU | A | 501 | −6.727 | −80.500 | −45.927 | 1.00 | 61.45 | N |
| ATOM | 3503 | CA | LEU | A | 501 | −5.477 | −81.158 | −46.298 | 1.00 | 61.49 | C |
| ATOM | 3504 | CB | LEU | A | 501 | −5.259 | −82.432 | −45.475 | 1.00 | 61.41 | C |
| ATOM | 3505 | CG | LEU | A | 501 | −5.099 | −82.291 | −43.959 | 1.00 | 61.24 | C |
| ATOM | 3506 | CD1 | LEU | A | 501 | −5.182 | −83.646 | −43.272 | 1.00 | 60.96 | C |
| ATOM | 3507 | CD2 | LEU | A | 501 | −3.792 | −81.603 | −43.617 | 1.00 | 61.45 | C |
| ATOM | 3508 | C | LEU | A | 501 | −5.480 | −81.494 | −47.788 | 1.00 | 61.59 | C |
| ATOM | 3509 | O | LEU | A | 501 | −6.352 | −82.220 | −48.264 | 1.00 | 61.52 | O |
| ATOM | 3510 | N | ASP | A | 502 | −4.515 | −80.952 | −48.525 | 1.00 | 61.72 | N |
| ATOM | 3511 | CA | ASP | A | 502 | −4.353 | −81.329 | −49.919 | 1.00 | 61.94 | C |
| ATOM | 3512 | CB | ASP | A | 502 | −3.736 | −80.192 | −50.739 | 1.00 | 62.06 | C |
| ATOM | 3513 | CG | ASP | A | 502 | −2.315 | −79.857 | −50.315 | 1.00 | 62.72 | C |
| ATOM | 3514 | OD1 | ASP | A | 502 | −1.539 | −80.784 | −49.993 | 1.00 | 63.46 | O |
| ATOM | 3515 | OD2 | ASP | A | 502 | −1.966 | −78.655 | −50.326 | 1.00 | 63.58 | O |
| ATOM | 3516 | C | ASP | A | 502 | −3.505 | −82.590 | −49.981 | 1.00 | 61.99 | C |
| ATOM | 3517 | O | ASP | A | 502 | −3.095 | −83.111 | −48.946 | 1.00 | 62.07 | O |
| ATOM | 3518 | N | ASP | A | 503 | −3.242 | −83.079 | −51.188 | 1.00 | 62.08 | N |
| ATOM | 3519 | CA | ASP | A | 503 | −2.516 | −84.337 | −51.362 | 1.00 | 62.21 | C |
| ATOM | 3520 | CB | ASP | A | 503 | −2.278 | −84.611 | −52.844 | 1.00 | 62.34 | C |
| ATOM | 3521 | CG | ASP | A | 503 | −1.514 | −83.495 | −53.525 | 1.00 | 62.98 | C |
| ATOM | 3522 | OD1 | ASP | A | 503 | −1.726 | −82.315 | −53.165 | 1.00 | 63.16 | O |
| ATOM | 3523 | OD2 | ASP | A | 503 | −0.703 | −83.803 | −54.425 | 1.00 | 64.06 | O |
| ATOM | 3524 | C | ASP | A | 503 | −1.193 | −84.407 | −50.593 | 1.00 | 62.12 | C |
| ATOM | 3525 | O | ASP | A | 503 | −0.955 | −85.360 | −49.843 | 1.00 | 62.04 | O |
| ATOM | 3526 | N | GLU | A | 504 | −0.337 | −83.405 | −50.779 | 1.00 | 62.02 | N |
| ATOM | 3527 | CA | GLU | A | 504 | 0.969 | −83.400 | −50.126 | 1.00 | 62.12 | C |
| ATOM | 3528 | CB | GLU | A | 504 | 1.813 | −82.201 | −50.574 | 1.00 | 62.02 | C |
| ATOM | 3529 | CG | GLU | A | 504 | 1.840 | −81.992 | −52.089 | 1.00 | 62.68 | C |
| ATOM | 3530 | CD | GLU | A | 504 | 3.004 | −81.120 | −52.556 | 1.00 | 62.81 | C |
| ATOM | 3531 | OE1 | GLU | A | 504 | 2.797 | −79.905 | −52.791 | 1.00 | 63.40 | O |
| ATOM | 3532 | OE2 | GLU | A | 504 | 4.130 | −81.652 | −52.694 | 1.00 | 63.95 | O |
| ATOM | 3533 | C | GLU | A | 504 | 0.793 | −83.410 | −48.614 | 1.00 | 61.85 | C |
| ATOM | 3534 | O | GLU | A | 504 | 1.286 | −84.308 | −47.927 | 1.00 | 61.84 | O |
| ATOM | 3535 | N | GLU | A | 505 | 0.069 | −82.420 | −48.102 | 1.00 | 61.64 | N |
| ATOM | 3536 | CA | GLU | A | 505 | −0.204 | −82.340 | −46.677 | 1.00 | 61.43 | C |
| ATOM | 3537 | CB | GLU | A | 505 | −1.201 | −81.228 | −46.390 | 1.00 | 61.18 | C |
| ATOM | 3538 | CG | GLU | A | 505 | −0.588 | −79.851 | −46.381 | 1.00 | 60.83 | C |
| ATOM | 3539 | CD | GLU | A | 505 | −1.631 | −78.753 | −46.286 | 1.00 | 60.55 | C |
| ATOM | 3540 | OE1 | GLU | A | 505 | −2.627 | −78.807 | −47.043 | 1.00 | 60.25 | O |
| ATOM | 3541 | OE2 | GLU | A | 505 | −1.457 | −77.832 | −45.456 | 1.00 | 60.26 | O |
| ATOM | 3542 | C | GLU | A | 505 | −0.735 | −83.667 | −46.149 | 1.00 | 61.67 | C |
| ATOM | 3543 | O | GLU | A | 505 | −0.357 | −84.106 | −45.056 | 1.00 | 61.83 | O |
| ATOM | 3544 | N | PHE | A | 506 | −1.604 | −84.309 | −46.927 | 1.00 | 61.68 | N |
| ATOM | 3545 | CA | PHE | A | 506 | −2.180 | −85.586 | −46.521 | 1.00 | 61.63 | C |
| ATOM | 3546 | CB | PHE | A | 506 | −3.294 | −86.020 | −47.474 | 1.00 | 61.56 | C |
| ATOM | 3547 | CG | PHE | A | 506 | −3.985 | −87.290 | −47.056 | 1.00 | 61.67 | C |
| ATOM | 3548 | CD1 | PHE | A | 506 | −3.470 | −88.528 | −47.414 | 1.00 | 61.62 | C |
| ATOM | 3549 | CE1 | PHE | A | 506 | −4.098 | −89.695 | −47.024 | 1.00 | 61.32 | C |
| ATOM | 3550 | CZ | PHE | A | 506 | −5.257 | −89.633 | −46.273 | 1.00 | 61.44 | C |
| ATOM | 3551 | CE2 | PHE | A | 506 | −5.781 | −88.409 | −45.912 | 1.00 | 61.14 | C |
| ATOM | 3552 | CD2 | PHE | A | 506 | −5.147 | −87.247 | −46.299 | 1.00 | 61.51 | C |
| ATOM | 3553 | C | PHE | A | 506 | −1.102 | −86.658 | −46.443 | 1.00 | 61.66 | C |
| ATOM | 3554 | O | PHE | A | 506 | −1.048 | −87.430 | −45.482 | 1.00 | 61.70 | O |
| ATOM | 3555 | N | ALA | A | 507 | −0.245 | −86.705 | −47.458 | 1.00 | 61.76 | N |
| ATOM | 3556 | CA | ALA | A | 507 | 0.876 | −87.636 | −47.447 | 1.00 | 61.66 | C |
| ATOM | 3557 | CB | ALA | A | 507 | 1.706 | −87.488 | −48.706 | 1.00 | 61.65 | C |
| ATOM | 3558 | C | ALA | A | 507 | 1.732 | −87.405 | −46.204 | 1.00 | 61.68 | C |
| ATOM | 3559 | O | ALA | A | 507 | 2.156 | −88.361 | −45.551 | 1.00 | 61.70 | O |
| ATOM | 3560 | N | LEU | A | 508 | 1.969 | −86.133 | −45.878 | 1.00 | 61.60 | N |
| ATOM | 3561 | CA | LEU | A | 508 | 2.695 | −85.767 | −44.661 | 1.00 | 61.54 | C |
| ATOM | 3562 | CB | LEU | A | 508 | 2.778 | −84.247 | −44.530 | 1.00 | 61.44 | C |
| ATOM | 3563 | CG | LEU | A | 508 | 4.018 | −83.623 | −43.886 | 1.00 | 61.08 | C |
| ATOM | 3564 | CD1 | LEU | A | 508 | 3.712 | −82.195 | −43.481 | 1.00 | 60.85 | C |
| ATOM | 3565 | CD2 | LEU | A | 508 | 4.505 | −84.417 | −42.691 | 1.00 | 60.65 | C |
| ATOM | 3566 | C | LEU | A | 508 | 1.991 | −86.346 | −43.433 | 1.00 | 61.70 | C |
| ATOM | 3567 | O | LEU | A | 508 | 2.595 | −87.063 | −42.621 | 1.00 | 61.61 | O |
| ATOM | 3568 | N | ALA | A | 509 | 0.708 | −86.026 | −43.301 | 1.00 | 61.77 | N |
| ATOM | 3569 | CA | ALA | A | 509 | −0.093 | −86.572 | −42.221 | 1.00 | 61.78 | C |
| ATOM | 3570 | CB | ALA | A | 509 | −1.560 | −86.243 | −42.434 | 1.00 | 61.77 | C |
| ATOM | 3571 | C | ALA | A | 509 | 0.122 | −88.082 | −42.115 | 1.00 | 61.87 | C |
| ATOM | 3572 | O | ALA | A | 509 | 0.550 | −88.585 | −41.073 | 1.00 | 61.97 | O |
| ATOM | 3573 | N | SER | A | 510 | −0.154 | −88.798 | −43.202 | 1.00 | 61.83 | N |
| ATOM | 3574 | CA | SER | A | 510 | 0.004 | −90.251 | −43.226 | 1.00 | 61.95 | C |
| ATOM | 3575 | CB | SER | A | 510 | −0.249 | −90.793 | −44.634 | 1.00 | 62.00 | C |
| ATOM | 3576 | OG | SER | A | 510 | −1.493 | −90.333 | −45.135 | 1.00 | 62.65 | O |
| ATOM | 3577 | C | SER | A | 510 | 1.390 | −90.673 | −42.748 | 1.00 | 61.79 | C |
| ATOM | 3578 | O | SER | A | 510 | 1.522 | −91.562 | −41.896 | 1.00 | 61.78 | O |
| ATOM | 3579 | N | HIS | A | 511 | 2.416 | −90.030 | −43.304 | 1.00 | 61.62 | N |
| ATOM | 3580 | CA | HIS | A | 511 | 3.793 | −90.302 | −42.913 | 1.00 | 61.40 | C |
| ATOM | 3581 | CB | HIS | A | 511 | 4.758 | −89.312 | −43.576 | 1.00 | 61.24 | C |
| ATOM | 3582 | CG | HIS | A | 511 | 6.183 | −89.470 | −43.139 | 1.00 | 60.84 | C |
| ATOM | 3583 | ND1 | HIS | A | 511 | 7.110 | −90.185 | −43.867 | 1.00 | 60.78 | N |
| ATOM | 3584 | CE1 | HIS | A | 511 | 8.275 | −90.155 | −43.244 | 1.00 | 60.24 | C |
| ATOM | 3585 | NE2 | HIS | A | 511 | 8.137 | −89.449 | −42.136 | 1.00 | 60.07 | N |
| ATOM | 3586 | CD2 | HIS | A | 511 | 6.838 | −89.009 | −42.047 | 1.00 | 60.36 | C |
| ATOM | 3587 | C | HIS | A | 511 | 3.906 | −90.212 | −41.404 | 1.00 | 61.38 | C |
| ATOM | 3588 | O | HIS | A | 511 | 4.408 | −91.124 | −40.751 | 1.00 | 61.56 | O |
| ATOM | 3589 | N | LEU | A | 512 | 3.417 | −89.109 | −40.852 | 1.00 | 61.36 | N |
| ATOM | 3590 | CA | LEU | A | 512 | 3.499 | −88.881 | −39.419 | 1.00 | 61.37 | C |
| ATOM | 3591 | CB | LEU | A | 512 | 3.011 | −87.469 | −39.097 | 1.00 | 61.22 | C |
| ATOM | 3592 | CG | LEU | A | 512 | 4.034 | −86.435 | −39.573 | 1.00 | 60.86 | C |
| ATOM | 3593 | CD1 | LEU | A | 512 | 3.482 | −85.018 | −39.516 | 1.00 | 60.51 | C |
| ATOM | 3594 | CD2 | LEU | A | 512 | 5.315 | −86.565 | −38.760 | 1.00 | 59.84 | C |
| ATOM | 3595 | C | LEU | A | 512 | 2.749 | −89.938 | −38.605 | 1.00 | 61.48 | C |
| ATOM | 3596 | O | LEU | A | 512 | 3.236 | −90.422 | −37.574 | 1.00 | 61.48 | O |
| ATOM | 3597 | N | ILE | A | 513 | 1.571 | −90.309 | −39.090 | 1.00 | 61.74 | N |
| ATOM | 3598 | CA | ILE | A | 513 | 0.791 | −91.371 | −38.468 | 1.00 | 61.83 | C |
| ATOM | 3599 | CB | ILE | A | 513 | −0.579 | −91.518 | −39.155 | 1.00 | 61.81 | C |
| ATOM | 3600 | CG1 | ILE | A | 513 | −1.325 | −90.181 | −39.084 | 1.00 | 61.73 | C |
| ATOM | 3601 | CD1 | ILE | A | 513 | −2.782 | −90.257 | −39.456 | 1.00 | 62.12 | C |
| ATOM | 3602 | CG2 | ILE | A | 513 | −1.382 | −92.645 | −38.518 | 1.00 | 61.68 | C |
| ATOM | 3603 | C | ILE | A | 513 | 1.554 | −92.705 | −38.444 | 1.00 | 61.89 | C |
| ATOM | 3604 | O | ILE | A | 513 | 1.683 | −93.333 | −37.391 | 1.00 | 61.85 | O |
| ATOM | 3605 | N | GLU | A | 514 | 2.071 | −93.124 | −39.598 | 1.00 | 61.92 | N |
| ATOM | 3606 | CA | GLU | A | 514 | 2.911 | −94.317 | −39.660 | 1.00 | 62.03 | C |
| ATOM | 3607 | CB | GLU | A | 514 | 3.456 | −94.519 | −41.069 | 1.00 | 62.08 | C |
| ATOM | 3608 | CG | GLU | A | 514 | 2.396 | −94.778 | −42.118 | 1.00 | 62.83 | C |
| ATOM | 3609 | CD | GLU | A | 514 | 2.944 | −95.511 | −43.333 | 1.00 | 63.54 | C |
| ATOM | 3610 | OE1 | GLU | A | 514 | 3.998 | −96.181 | −43.216 | 1.00 | 63.39 | O |
| ATOM | 3611 | OE2 | GLU | A | 514 | 2.310 | −95.420 | −44.407 | 1.00 | 64.11 | O |
| ATOM | 3612 | C | GLU | A | 514 | 4.077 | −94.201 | −38.689 | 1.00 | 62.07 | C |
| ATOM | 3613 | O | GLU | A | 514 | 4.345 | −95.108 | −37.894 | 1.00 | 62.33 | O |
| ATOM | 3614 | N | ALA | A | 515 | 4.775 | −93.072 | −38.767 | 1.00 | 61.99 | N |
| ATOM | 3615 | CA | ALA | A | 515 | 5.881 | −92.794 | −37.865 | 1.00 | 61.95 | C |
| ATOM | 3616 | CB | ALA | A | 515 | 6.338 | −91.346 | −38.007 | 1.00 | 61.86 | C |
| ATOM | 3617 | C | ALA | A | 515 | 5.477 | −93.091 | −36.426 | 1.00 | 61.91 | C |
| ATOM | 3618 | O | ALA | A | 515 | 6.134 | −93.878 | −35.746 | 1.00 | 61.89 | O |
| ATOM | 3619 | N | LYS | A | 516 | 4.389 | −92.472 | −35.972 | 1.00 | 61.87 | N |
| ATOM | 3620 | CA | LYS | A | 516 | 3.946 | −92.658 | −34.592 | 1.00 | 61.79 | C |
| ATOM | 3621 | CB | LYS | A | 516 | 2.753 | −91.760 | −34.258 | 1.00 | 61.68 | C |
| ATOM | 3622 | CG | LYS | A | 516 | 2.874 | −91.054 | −32.915 | 1.00 | 61.28 | C |
| ATOM | 3623 | CD | LYS | A | 516 | 2.859 | −92.024 | −31.750 | 1.00 | 61.20 | C |
| ATOM | 3624 | CE | LYS | A | 516 | 3.802 | −91.567 | −30.647 | 1.00 | 61.34 | C |
| ATOM | 3625 | NZ | LYS | A | 516 | 3.687 | −90.108 | −30.377 | 1.00 | 61.19 | N |
| ATOM | 3626 | C | LYS | A | 516 | 3.598 | −94.113 | −34.336 | 1.00 | 61.88 | C |
| ATOM | 3627 | O | LYS | A | 516 | 3.898 | −94.645 | −33.269 | 1.00 | 62.04 | O |
| ATOM | 3628 | N | LEU | A | 517 | 2.975 | −94.761 | −35.315 | 1.00 | 61.97 | N |
| ATOM | 3629 | CA | LEU | A | 517 | 2.643 | −96.176 | −35.178 | 1.00 | 62.25 | C |
| ATOM | 3630 | CB | LEU | A | 517 | 1.868 | −96.690 | −36.396 | 1.00 | 62.24 | C |
| ATOM | 3631 | CG | LEU | A | 517 | 0.436 | −96.164 | −36.551 | 1.00 | 62.30 | C |
| ATOM | 3632 | CD1 | LEU | A | 517 | −0.225 | −96.729 | −37.800 | 1.00 | 62.24 | C |
| ATOM | 3633 | CD2 | LEU | A | 517 | −0.395 | −96.473 | −35.313 | 1.00 | 62.23 | C |
| ATOM | 3634 | C | LEU | A | 517 | 3.893 | −97.019 | −34.935 | 1.00 | 62.46 | C |
| ATOM | 3635 | O | LEU | A | 517 | 3.871 | −97.939 | −34.123 | 1.00 | 62.42 | O |
| ATOM | 3636 | N | GLU | A | 518 | 4.983 | −96.698 | −35.628 | 1.00 | 62.79 | N |
| ATOM | 3637 | CA | GLU | A | 518 | 6.252 | −97.404 | −35.418 | 1.00 | 63.31 | C |
| ATOM | 3638 | CB | GLU | A | 518 | 7.314 | −96.944 | −36.421 | 1.00 | 63.45 | C |
| ATOM | 3639 | CG | GLU | A | 518 | 7.738 | −98.010 | −37.419 | 1.00 | 63.87 | C |
| ATOM | 3640 | CD | GLU | A | 518 | 6.782 | −98.127 | −38.581 | 1.00 | 64.37 | C |
| ATOM | 3641 | OE1 | GLU | A | 518 | 6.048 | −99.135 | −38.645 | 1.00 | 64.42 | O |
| ATOM | 3642 | OE2 | GLU | A | 518 | 6.759 | −97.205 | −39.426 | 1.00 | 64.82 | O |
| ATOM | 3643 | C | GLU | A | 518 | 6.799 | −97.252 | −34.001 | 1.00 | 63.54 | C |
| ATOM | 3644 | O | GLU | A | 518 | 7.571 | −98.091 | −33.532 | 1.00 | 63.50 | O |
| ATOM | 3645 | N | GLY | A | 519 | 6.406 | −96.175 | −33.329 | 1.00 | 63.92 | N |
| ATOM | 3646 | CA | GLY | A | 519 | 6.916 | −95.872 | −31.995 | 1.00 | 64.42 | C |
| ATOM | 3647 | C | GLY | A | 519 | 7.909 | −94.725 | −32.008 | 1.00 | 64.74 | C |
| ATOM | 3648 | O | GLY | A | 519 | 9.012 | −94.834 | −31.468 | 1.00 | 64.74 | O |
| ATOM | 3649 | N | HIS | A | 520 | 7.513 | −93.620 | −32.633 | 1.00 | 65.16 | N |
| ATOM | 3650 | CA | HIS | A | 520 | 8.361 | −92.438 | −32.716 | 1.00 | 65.57 | C |
| ATOM | 3651 | CB | HIS | A | 520 | 8.735 | −92.143 | −34.169 | 1.00 | 65.67 | C |
| ATOM | 3652 | CG | HIS | A | 520 | 9.840 | −93.003 | −34.691 | 1.00 | 66.10 | C |
| ATOM | 3653 | ND1 | HIS | A | 520 | 11.163 | −92.796 | −34.357 | 1.00 | 66.58 | N |
| ATOM | 3654 | CE1 | HIS | A | 520 | 11.911 | −93.707 | −34.955 | 1.00 | 66.72 | C |
| ATOM | 3655 | NE2 | HIS | A | 520 | 11.121 | −94.495 | −35.666 | 1.00 | 66.89 | N |
| ATOM | 3656 | CD2 | HIS | A | 520 | 9.820 | −94.077 | −35.517 | 1.00 | 66.36 | C |
| ATOM | 3657 | C | HIS | A | 520 | 7.678 | −91.219 | −32.126 | 1.00 | 65.70 | C |
| ATOM | 3658 | O | HIS | A | 520 | 6.576 | −90.855 | −32.538 | 1.00 | 65.77 | O |
| ATOM | 3659 | N | GLY | A | 521 | 8.335 | −90.583 | −31.163 | 1.00 | 65.86 | N |
| ATOM | 3660 | CA | GLY | A | 521 | 7.867 | −89.296 | −30.678 | 1.00 | 66.01 | C |
| ATOM | 3661 | C | GLY | A | 521 | 7.715 | −88.376 | −31.873 | 1.00 | 66.07 | C |
| ATOM | 3662 | O | GLY | A | 521 | 8.524 | −88.416 | −32.802 | 1.00 | 66.13 | O |
| ATOM | 3663 | N | LEU | A | 522 | 6.663 | −87.569 | −31.874 | 1.00 | 66.10 | N |
| ATOM | 3664 | CA | LEU | A | 522 | 6.483 | −86.582 | −32.929 | 1.00 | 66.04 | C |
| ATOM | 3665 | CB | LEU | A | 522 | 5.019 | −86.174 | −33.043 | 1.00 | 66.08 | C |
| ATOM | 3666 | CG | LEU | A | 522 | 4.179 | −86.795 | −34.157 | 1.00 | 66.47 | C |
| ATOM | 3667 | CD1 | LEU | A | 522 | 4.642 | −88.202 | −34.519 | 1.00 | 66.89 | C |
| ATOM | 3668 | CD2 | LEU | A | 522 | 2.724 | −86.798 | −33.720 | 1.00 | 66.94 | C |
| ATOM | 3669 | C | LEU | A | 522 | 7.322 | −85.351 | −32.639 | 1.00 | 65.97 | C |
| ATOM | 3670 | O | LEU | A | 522 | 7.534 | −84.999 | −31.478 | 1.00 | 65.87 | O |
| ATOM | 3671 | N | PRO | A | 523 | 7.808 | −84.690 | −33.699 | 1.00 | 65.92 | N |
| ATOM | 3672 | CA | PRO | A | 523 | 8.473 | −83.409 | −33.516 | 1.00 | 65.84 | C |
| ATOM | 3673 | CB | PRO | A | 523 | 8.849 | −82.993 | −34.943 | 1.00 | 65.78 | C |
| ATOM | 3674 | CG | PRO | A | 523 | 8.792 | −84.241 | −35.750 | 1.00 | 65.95 | C |
| ATOM | 3675 | CD | PRO | A | 523 | 7.751 | −85.097 | −35.113 | 1.00 | 65.96 | C |
| ATOM | 3676 | C | PRO | A | 523 | 7.476 | −82.414 | −32.939 | 1.00 | 65.77 | C |
| ATOM | 3677 | O | PRO | A | 523 | 6.272 | −82.685 | −32.901 | 1.00 | 65.97 | O |
| ATOM | 3678 | N | THR | A | 524 | 7.965 | −81.272 | −32.484 | 1.00 | 65.48 | N |
| ATOM | 3679 | CA | THR | A | 524 | 7.065 | −80.238 | −32.020 | 1.00 | 65.17 | C |
| ATOM | 3680 | CB | THR | A | 524 | 7.481 | −79.717 | −30.623 | 1.00 | 65.20 | C |
| ATOM | 3681 | OG1 | THR | A | 524 | 7.177 | −78.323 | −30.514 | 1.00 | 65.65 | O |
| ATOM | 3682 | CG2 | THR | A | 524 | 8.974 | −79.937 | −30.389 | 1.00 | 65.23 | C |
| ATOM | 3683 | C | THR | A | 524 | 6.952 | −79.137 | −33.084 | 1.00 | 64.84 | C |
| ATOM | 3684 | O | THR | A | 524 | 5.974 | −78.392 | −33.122 | 1.00 | 64.93 | O |
| ATOM | 3685 | N | ASN | A | 525 | 7.946 | −79.070 | −33.966 | 1.00 | 64.41 | N |
| ATOM | 3686 | CA | ASN | A | 525 | 7.941 | −78.137 | −35.095 | 1.00 | 63.87 | C |
| ATOM | 3687 | CB | ASN | A | 525 | 8.818 | −76.920 | −34.805 | 1.00 | 63.88 | C |
| ATOM | 3688 | CG | ASN | A | 525 | 8.167 | −75.948 | −33.854 | 1.00 | 63.78 | C |
| ATOM | 3689 | OD1 | ASN | A | 525 | 8.737 | −75.603 | −32.820 | 1.00 | 63.84 | O |
| ATOM | 3690 | ND2 | ASN | A | 525 | 6.961 | −75.502 | −34.194 | 1.00 | 63.55 | N |
| ATOM | 3691 | C | ASN | A | 525 | 8.422 | −78.805 | −36.374 | 1.00 | 63.51 | C |
| ATOM | 3692 | O | ASN | A | 525 | 9.530 | −79.340 | −36.428 | 1.00 | 63.55 | O |
| ATOM | 3693 | N | LEU | A | 526 | 7.583 | −78.761 | −37.401 | 1.00 | 62.99 | N |
| ATOM | 3694 | CA | LEU | A | 526 | 7.882 | −79.378 | −38.681 | 1.00 | 62.43 | C |
| ATOM | 3695 | CB | LEU | A | 526 | 6.694 | −79.165 | −39.615 | 1.00 | 62.46 | C |
| ATOM | 3696 | CG | LEU | A | 526 | 6.369 | −80.239 | −40.650 | 1.00 | 62.26 | C |
| ATOM | 3697 | CD1 | LEU | A | 526 | 7.395 | −80.225 | −41.753 | 1.00 | 62.66 | C |
| ATOM | 3698 | CD2 | LEU | A | 526 | 6.303 | −81.602 | −39.996 | 1.00 | 62.13 | C |
| ATOM | 3699 | C | LEU | A | 526 | 9.146 | −78.766 | −39.286 | 1.00 | 62.24 | C |
| ATOM | 3700 | O | LEU | A | 526 | 9.145 | −77.599 | −39.674 | 1.00 | 62.21 | O |
| ATOM | 3701 | N | PRO | A | 527 | 10.234 | −79.554 | −39.371 | 1.00 | 61.99 | N |
| ATOM | 3702 | CA | PRO | A | 527 | 11.486 | −79.062 | −39.942 | 1.00 | 61.70 | C |
| ATOM | 3703 | CB | PRO | A | 527 | 12.503 | −80.112 | −39.495 | 1.00 | 61.70 | C |
| ATOM | 3704 | CG | PRO | A | 527 | 11.722 | −81.364 | −39.395 | 1.00 | 61.61 | C |
| ATOM | 3705 | CD | PRO | A | 527 | 10.340 | −80.961 | −38.941 | 1.00 | 61.99 | C |
| ATOM | 3706 | C | PRO | A | 527 | 11.417 | −79.005 | −41.466 | 1.00 | 61.51 | C |
| ATOM | 3707 | O | PRO | A | 527 | 10.651 | −79.751 | −42.079 | 1.00 | 61.49 | O |
| ATOM | 3708 | N | ARG | A | 528 | 12.221 | −78.133 | −42.069 | 1.00 | 61.27 | N |
| ATOM | 3709 | CA | ARG | A | 528 | 12.154 | −77.888 | −43.510 | 1.00 | 61.10 | C |
| ATOM | 3710 | CB | ARG | A | 528 | 13.334 | −77.024 | −43.968 | 1.00 | 61.10 | C |
| ATOM | 3711 | CG | ARG | A | 528 | 13.363 | −76.776 | −45.472 | 1.00 | 61.06 | C |
| ATOM | 3712 | CD | ARG | A | 528 | 14.621 | −76.043 | −45.903 | 1.00 | 61.02 | C |
| ATOM | 3713 | NE | ARG | A | 528 | 14.650 | −75.823 | −47.347 | 1.00 | 60.86 | N |
| ATOM | 3714 | CZ | ARG | A | 528 | 14.282 | −74.691 | −47.945 | 1.00 | 60.65 | C |
| ATOM | 3715 | NH1 | ARG | A | 528 | 14.343 | −74.592 | −49.267 | 1.00 | 60.39 | N |
| ATOM | 3716 | NH2 | ARG | A | 528 | 13.855 | −73.658 | −47.227 | 1.00 | 60.55 | N |
| ATOM | 3717 | C | ARG | A | 528 | 12.091 | −79.163 | −44.355 | 1.00 | 61.02 | C |
| ATOM | 3718 | O | ARG | A | 528 | 11.282 | −79.270 | −45.277 | 1.00 | 60.96 | O |
| ATOM | 3719 | N | ARG | A | 529 | 12.948 | −80.125 | −44.032 | 1.00 | 60.92 | N |
| ATOM | 3720 | CA | ARG | A | 529 | 13.097 | −81.325 | −44.848 | 1.00 | 60.87 | C |
| ATOM | 3721 | CB | ARG | A | 529 | 14.249 | −82.189 | −44.328 | 1.00 | 61.05 | C |
| ATOM | 3722 | CG | ARG | A | 529 | 14.118 | −82.565 | −42.867 | 1.00 | 61.92 | C |
| ATOM | 3723 | CD | ARG | A | 529 | 15.338 | −83.315 | −42.367 | 1.00 | 63.40 | C |
| ATOM | 3724 | NE | ARG | A | 529 | 15.130 | −83.798 | −41.006 | 1.00 | 64.56 | N |
| ATOM | 3725 | CZ | ARG | A | 529 | 14.453 | −84.901 | −40.702 | 1.00 | 65.44 | C |
| ATOM | 3726 | NH1 | ARG | A | 529 | 14.311 | −85.259 | −39.432 | 1.00 | 65.89 | N |
| ATOM | 3727 | NH2 | ARG | A | 529 | 13.916 | −85.646 | −41.665 | 1.00 | 65.76 | N |
| ATOM | 3728 | C | ARG | A | 529 | 11.817 | −82.142 | −44.925 | 1.00 | 60.59 | C |
| ATOM | 3729 | O | ARG | A | 529 | 11.665 | −82.979 | −45.817 | 1.00 | 60.74 | O |
| ATOM | 3730 | N | LEU | A | 530 | 10.901 | −81.907 | −43.991 | 1.00 | 60.16 | N |
| ATOM | 3731 | CA | LEU | A | 530 | 9.623 | −82.605 | −44.003 | 1.00 | 59.71 | C |
| ATOM | 3732 | CB | LEU | A | 530 | 9.209 | −83.023 | −42.591 | 1.00 | 59.65 | C |
| ATOM | 3733 | CG | LEU | A | 530 | 9.701 | −84.382 | −42.092 | 1.00 | 59.14 | C |
| ATOM | 3734 | CD1 | LEU | A | 530 | 9.103 | −84.693 | −40.730 | 1.00 | 58.59 | C |
| ATOM | 3735 | CD2 | LEU | A | 530 | 9.345 | −85.472 | −43.082 | 1.00 | 58.67 | C |
| ATOM | 3736 | C | LEU | A | 530 | 8.520 | −81.784 | −44.664 | 1.00 | 59.56 | C |
| ATOM | 3737 | O | LEU | A | 530 | 7.468 | −82.318 | −45.009 | 1.00 | 59.51 | O |
| ATOM | 3738 | N | VAL | A | 531 | 8.756 | −80.488 | −44.840 | 1.00 | 59.45 | N |
| ATOM | 3739 | CA | VAL | A | 531 | 7.784 | −79.637 | −45.519 | 1.00 | 59.44 | C |
| ATOM | 3740 | CB | VAL | A | 531 | 8.167 | −78.142 | −45.438 | 1.00 | 59.28 | C |
| ATOM | 3741 | CG1 | VAL | A | 531 | 7.067 | −77.283 | −46.024 | 1.00 | 58.89 | C |
| ATOM | 3742 | CG2 | VAL | A | 531 | 8.443 | −77.735 | −44.006 | 1.00 | 59.32 | C |
| ATOM | 3743 | C | VAL | A | 531 | 7.685 | −80.033 | −46.989 | 1.00 | 59.61 | C |
| ATOM | 3744 | O | VAL | A | 531 | 8.700 | −80.081 | −47.685 | 1.00 | 59.64 | O |
| ATOM | 3745 | N | PRO | A | 532 | 6.464 | −80.329 | −47.467 | 1.00 | 59.71 | N |
| ATOM | 3746 | CA | PRO | A | 532 | 6.282 | −80.580 | −48.891 | 1.00 | 59.85 | C |
| ATOM | 3747 | CB | PRO | A | 532 | 4.776 | −80.410 | −49.079 | 1.00 | 59.82 | C |
| ATOM | 3748 | CG | PRO | A | 532 | 4.193 | −80.816 | −47.766 | 1.00 | 59.65 | C |
| ATOM | 3749 | CD | PRO | A | 532 | 5.203 | −80.447 | −46.713 | 1.00 | 59.60 | C |
| ATOM | 3750 | C | PRO | A | 532 | 7.044 | −79.537 | −49.707 | 1.00 | 60.13 | C |
| ATOM | 3751 | O | PRO | A | 532 | 7.085 | −78.370 | −49.322 | 1.00 | 60.15 | O |
| ATOM | 3752 | N | PRO | A | 533 | 7.648 | −79.960 | −50.829 | 1.00 | 60.38 | N |
| ATOM | 3753 | CA | PRO | A | 533 | 8.535 | −79.145 | −51.659 | 1.00 | 60.52 | C |
| ATOM | 3754 | CB | PRO | A | 533 | 8.659 | −79.971 | −52.938 | 1.00 | 60.55 | C |
| ATOM | 3755 | CG | PRO | A | 533 | 8.543 | −81.366 | −52.458 | 1.00 | 60.51 | C |
| ATOM | 3756 | CD | PRO | A | 533 | 7.498 | −81.321 | −51.373 | 1.00 | 60.41 | C |
| ATOM | 3757 | C | PRO | A | 533 | 8.031 | −77.729 | −51.965 | 1.00 | 60.72 | C |
| ATOM | 3758 | O | PRO | A | 533 | 8.842 | −76.825 | −52.156 | 1.00 | 60.82 | O |
| ATOM | 3759 | N | SER | A | 534 | 6.717 | −77.532 | −52.022 | 1.00 | 61.01 | N |
| ATOM | 3760 | CA | SER | A | 534 | 6.176 | −76.176 | −52.028 | 1.00 | 61.33 | C |
| ATOM | 3761 | CB | SER | A | 534 | 4.719 | −76.179 | −52.469 | 1.00 | 61.27 | C |
| ATOM | 3762 | OG | SER | A | 534 | 4.535 | −77.089 | −53.541 | 1.00 | 61.06 | O |
| ATOM | 3763 | C | SER | A | 534 | 6.320 | −75.634 | −50.606 | 1.00 | 61.73 | C |
| ATOM | 3764 | O | SER | A | 534 | 5.342 | −75.532 | −49.849 | 1.00 | 61.49 | O |
| ATOM | 3765 | N | LYS | A | 535 | 7.561 | −75.302 | −50.248 | 1.00 | 62.26 | N |
| ATOM | 3766 | CA | LYS | A | 535 | 7.909 | −75.061 | −48.849 | 1.00 | 62.66 | C |
| ATOM | 3767 | CB | LYS | A | 535 | 9.019 | −76.020 | −48.362 | 1.00 | 62.74 | C |
| ATOM | 3768 | CG | LYS | A | 535 | 10.263 | −76.121 | −49.239 | 1.00 | 62.63 | C |
| ATOM | 3769 | CD | LYS | A | 535 | 11.276 | −77.127 | −48.668 | 1.00 | 62.53 | C |
| ATOM | 3770 | CE | LYS | A | 535 | 10.889 | −78.574 | −48.974 | 1.00 | 62.10 | C |
| ATOM | 3771 | NZ | LYS | A | 535 | 11.955 | −79.555 | −48.612 | 1.00 | 61.50 | N |
| ATOM | 3772 | C | LYS | A | 535 | 8.221 | −73.624 | −48.467 | 1.00 | 62.89 | C |
| ATOM | 3773 | O | LYS | A | 535 | 7.423 | −73.007 | −47.763 | 1.00 | 63.20 | O |
| ATOM | 3774 | N | ARG | A | 536 | 9.356 | −73.086 | −48.915 | 1.00 | 62.87 | N |
| ATOM | 3775 | CA | ARG | A | 536 | 9.820 | −71.790 | −48.411 | 1.00 | 62.96 | C |
| ATOM | 3776 | CB | ARG | A | 536 | 10.355 | −71.935 | −46.981 | 1.00 | 63.00 | C |
| ATOM | 3777 | CG | ARG | A | 536 | 9.589 | −71.160 | −45.920 | 1.00 | 62.88 | C |
| ATOM | 3778 | CD | ARG | A | 536 | 10.104 | −69.730 | −45.785 | 1.00 | 62.78 | C |
| ATOM | 3779 | NE | ARG | A | 536 | 9.513 | −69.039 | −44.637 | 1.00 | 62.80 | N |
| ATOM | 3780 | CZ | ARG | A | 536 | 9.662 | −67.741 | −44.378 | 1.00 | 62.71 | C |
| ATOM | 3781 | NH1 | ARG | A | 536 | 9.085 | −67.202 | −43.312 | 1.00 | 62.64 | N |
| ATOM | 3782 | NH2 | ARG | A | 536 | 10.385 | −66.977 | −45.186 | 1.00 | 62.73 | N |
| ATOM | 3783 | C | ARG | A | 536 | 10.907 | −71.172 | −49.261 | 1.00 | 63.09 | C |
| ATOM | 3784 | O | ARG | A | 536 | 12.064 | −71.595 | −49.195 | 1.00 | 63.09 | O |
| ATOM | 3785 | N | ARG | A | 537 | 10.536 | −70.154 | −50.040 | 1.00 | 63.37 | N |
| ATOM | 3786 | CA | ARG | A | 537 | 11.507 | −69.382 | −50.808 | 1.00 | 63.60 | C |
| ATOM | 3787 | CB | ARG | A | 537 | 10.803 | −68.242 | −51.556 | 1.00 | 63.66 | C |
| ATOM | 3788 | CG | ARG | A | 537 | 11.370 | −67.921 | −52.949 | 1.00 | 64.02 | C |
| ATOM | 3789 | CD | ARG | A | 537 | 12.815 | −67.398 | −52.901 | 1.00 | 64.23 | C |
| ATOM | 3790 | NE | ARG | A | 537 | 13.276 | −66.952 | −54.216 | 1.00 | 64.87 | N |
| ATOM | 3791 | CZ | ARG | A | 537 | 13.586 | −65.692 | −54.516 | 1.00 | 65.30 | C |
| ATOM | 3792 | NH1 | ARG | A | 537 | 13.493 | −64.746 | −53.591 | 1.00 | 65.61 | N |
| ATOM | 3793 | NH2 | ARG | A | 537 | 13.998 | −65.373 | −55.739 | 1.00 | 64.97 | N |
| ATOM | 3794 | C | ARG | A | 537 | 12.530 | −68.816 | −49.820 | 1.00 | 63.69 | C |
| ATOM | 3795 | O | ARG | A | 537 | 12.202 | −68.553 | −48.657 | 1.00 | 63.76 | O |
| ATOM | 3796 | N | GLN | A | 538 | 13.766 | −68.641 | −50.275 | 1.00 | 63.75 | N |
| ATOM | 3797 | CA | GLN | A | 538 | 14.834 | −68.177 | −49.394 | 1.00 | 63.87 | C |
| ATOM | 3798 | CB | GLN | A | 538 | 15.457 | −69.363 | −48.658 | 1.00 | 63.84 | C |
| ATOM | 3799 | CG | GLN | A | 538 | 15.906 | −70.488 | −49.582 | 1.00 | 63.92 | C |
| ATOM | 3800 | CD | GLN | A | 538 | 16.296 | −71.738 | −48.817 | 1.00 | 64.01 | C |
| ATOM | 3801 | OE1 | GLN | A | 538 | 16.303 | −71.751 | −47.584 | 1.00 | 64.15 | O |
| ATOM | 3802 | NE2 | GLN | A | 538 | 16.622 | −72.802 | −49.545 | 1.00 | 64.37 | N |
| ATOM | 3803 | C | GLN | A | 538 | 15.909 | −67.416 | −50.160 | 1.00 | 63.88 | C |
| ATOM | 3804 | O | GLN | A | 538 | 17.043 | −67.243 | −49.647 | 1.00 | 63.92 | O |
| ATOM | 3805 | O2A | ANP | A | 600 | 11.527 | −56.574 | −1.805 | 1.00 | 48.61 | O |
| ATOM | 3806 | PA | ANP | A | 600 | 11.448 | −57.106 | −3.213 | 1.00 | 48.08 | P |
| ATOM | 3807 | O1A | ANP | A | 600 | 12.590 | −57.937 | −3.742 | 1.00 | 48.41 | O |
| ATOM | 3808 | O3A | ANP | A | 600 | 11.221 | −55.854 | −4.189 | 1.00 | 49.01 | O |
| ATOM | 3809 | PB | ANP | A | 600 | 11.878 | −54.443 | −3.787 | 1.00 | 49.55 | P |
| ATOM | 3810 | O1B | ANP | A | 600 | 12.072 | −53.654 | −5.058 | 1.00 | 49.44 | O |
| ATOM | 3811 | O2B | ANP | A | 600 | 13.088 | −54.721 | −2.926 | 1.00 | 49.32 | O |
| ATOM | 3812 | N3B | ANP | A | 600 | 10.688 | −53.641 | −2.803 | 1.00 | 49.69 | N |
| ATOM | 3813 | PG | ANP | A | 600 | 11.161 | −52.172 | −2.006 | 1.00 | 50.29 | P |
| ATOM | 3814 | O3G | ANP | A | 600 | 11.033 | −51.114 | −3.076 | 1.00 | 50.68 | O |
| ATOM | 3815 | O2G | ANP | A | 600 | 10.150 | −52.031 | −0.893 | 1.00 | 50.99 | O |
| ATOM | 3816 | O1G | ANP | A | 600 | 12.579 | −52.435 | −1.551 | 1.00 | 49.88 | O |
| ATOM | 3817 | O5* | ANP | A | 600 | 10.094 | −57.950 | −3.373 | 1.00 | 48.33 | O |
| ATOM | 3818 | C5* | ANP | A | 600 | 9.065 | −57.592 | −4.286 | 1.00 | 49.10 | C |
| ATOM | 3819 | C4* | ANP | A | 600 | 7.956 | −58.626 | −4.134 | 1.00 | 50.21 | C |
| ATOM | 3820 | C3* | ANP | A | 600 | 8.309 | −59.661 | −3.080 | 1.00 | 50.31 | C |
| ATOM | 3821 | O3* | ANP | A | 600 | 7.105 | −60.157 | −2.491 | 1.00 | 50.26 | O |
| ATOM | 3822 | C2* | ANP | A | 600 | 8.963 | −60.782 | −3.853 | 1.00 | 50.21 | C |
| ATOM | 3823 | O2* | ANP | A | 600 | 8.705 | −62.044 | −3.239 | 1.00 | 50.10 | O |
| ATOM | 3824 | C1* | ANP | A | 600 | 8.291 | −60.692 | −5.208 | 1.00 | 50.66 | C |
| ATOM | 3825 | O4* | ANP | A | 600 | 7.807 | −59.354 | −5.353 | 1.00 | 50.80 | O |
| ATOM | 3826 | N9 | ANP | A | 600 | 9.253 | −60.938 | −6.296 | 1.00 | 50.73 | N |
| ATOM | 3827 | C4 | ANP | A | 600 | 8.946 | −61.536 | −7.443 | 1.00 | 50.52 | C |
| ATOM | 3828 | C5 | ANP | A | 600 | 10.180 | −61.565 | −8.228 | 1.00 | 50.67 | C |
| ATOM | 3829 | N7 | ANP | A | 600 | 11.130 | −60.971 | −7.480 | 1.00 | 51.15 | N |
| ATOM | 3830 | C8 | ANP | A | 600 | 10.552 | −60.592 | −6.309 | 1.00 | 50.79 | C |
| ATOM | 3831 | N3 | ANP | A | 600 | 7.823 | −62.071 | −7.962 | 1.00 | 50.46 | N |
| ATOM | 3832 | C2 | ANP | A | 600 | 7.828 | −62.625 | −9.184 | 1.00 | 50.75 | C |
| ATOM | 3833 | N1 | ANP | A | 600 | 8.920 | −62.692 | −9.963 | 1.00 | 50.79 | N |
| ATOM | 3834 | C6 | ANP | A | 600 | 10.108 | −62.188 | −9.564 | 1.00 | 50.80 | C |
| ATOM | 3835 | N6 | ANP | A | 600 | 11.205 | −62.253 | −10.356 | 1.00 | 50.98 | N |
| ATOM | 3836 | MG | MG | A | 700 | 13.847 | −53.618 | −0.877 | 1.00 | 49.55 | MG |
| ATOM | 3837 | CA | CA | A | 701 | −4.087 | −77.142 | −45.879 | 1.00 | 46.71 | CA |
| ATOM | 1 | N | ARG | B | 19 | −35.864 | −44.695 | −40.362 | 1.00 | 58.79 | N |
| ATOM | 2 | CA | ARG | B | 19 | −36.349 | −44.622 | −38.954 | 1.00 | 58.86 | C |
| ATOM | 3 | CB | ARG | B | 19 | −37.709 | −45.314 | −38.819 | 1.00 | 58.86 | C |
| ATOM | 4 | CG | ARG | B | 19 | −37.648 | −46.838 | −38.872 | 1.00 | 59.05 | C |
| ATOM | 5 | CD | ARG | B | 19 | −39.035 | −47.464 | −38.760 | 1.00 | 59.14 | C |
| ATOM | 6 | NE | ARG | B | 19 | −39.799 | −46.949 | −37.622 | 1.00 | 59.75 | N |
| ATOM | 7 | CZ | ARG | B | 19 | −40.800 | −46.076 | −37.722 | 1.00 | 59.95 | C |
| ATOM | 8 | NH1 | ARG | B | 19 | −41.169 | −45.613 | −38.911 | 1.00 | 60.07 | N |
| ATOM | 9 | NH2 | ARG | B | 19 | −41.437 | −45.665 | −36.631 | 1.00 | 59.86 | N |
| ATOM | 10 | C | ARG | B | 19 | −35.335 | −45.249 | −37.999 | 1.00 | 58.73 | C |
| ATOM | 11 | O | ARG | B | 19 | −35.582 | −45.372 | −36.800 | 1.00 | 58.69 | O |
| ATOM | 1 | N | THR | B | 20 | −34.188 | −45.636 | −38.545 | 1.00 | 58.62 | N |
| ATOM | 2 | CA | THR | B | 20 | −33.114 | −46.223 | −37.751 | 1.00 | 58.46 | C |
| ATOM | 3 | CB | THR | B | 20 | −32.290 | −47.226 | −38.581 | 1.00 | 58.50 | C |
| ATOM | 4 | OG1 | THR | B | 20 | −31.604 | −46.534 | −39.634 | 1.00 | 58.37 | O |
| ATOM | 5 | CG2 | THR | B | 20 | −33.199 | −48.292 | −39.178 | 1.00 | 58.59 | C |
| ATOM | 6 | C | THR | B | 20 | −32.180 | −45.152 | −37.186 | 1.00 | 58.32 | C |
| ATOM | 7 | O | THR | B | 20 | −32.520 | −43.965 | −37.165 | 1.00 | 58.25 | O |
| ATOM | 1 | N | VAL | B | 21 | −31.000 | −45.579 | −36.740 | 1.00 | 58.12 | N |
| ATOM | 2 | CA | VAL | B | 21 | −30.048 | −44.683 | −36.083 | 1.00 | 57.89 | C |
| ATOM | 3 | CB | VAL | B | 21 | −29.111 | −45.447 | −35.127 | 1.00 | 57.93 | C |
| ATOM | 4 | CG1 | VAL | B | 21 | −28.124 | −46.293 | −35.915 | 1.00 | 58.17 | C |
| ATOM | 5 | CG2 | VAL | B | 21 | −28.372 | −44.474 | −34.220 | 1.00 | 57.98 | C |
| ATOM | 6 | C | VAL | B | 21 | −29.212 | −43.859 | −37.066 | 1.00 | 57.68 | C |
| ATOM | 7 | O | VAL | B | 21 | −29.074 | −42.644 | −36.900 | 1.00 | 57.39 | O |
| ATOM | 1 | N | THR | B | 22 | −28.652 | −44.511 | −38.082 | 1.00 | 57.46 | N |
| ATOM | 2 | CA | THR | B | 22 | −27.859 | −43.792 | −39.077 | 1.00 | 57.30 | C |
| ATOM | 3 | CB | THR | B | 22 | −27.161 | −44.740 | −40.090 | 1.00 | 57.27 | C |
| ATOM | 4 | OG1 | THR | B | 22 | −28.023 | −44.987 | −41.207 | 1.00 | 57.51 | O |
| ATOM | 5 | CG2 | THR | B | 22 | −26.780 | −46.062 | −39.430 | 1.00 | 57.23 | C |
| ATOM | 6 | C | THR | B | 22 | −28.755 | −42.784 | −39.798 | 1.00 | 57.11 | C |
| ATOM | 7 | O | THR | B | 22 | −28.337 | −41.669 | −40.100 | 1.00 | 57.00 | O |
| ATOM | 1 | N | SER | B | 23 | −29.994 | −43.183 | −40.059 | 1.00 | 56.93 | N |
| ATOM | 2 | CA | SER | B | 23 | −31.000 | −42.275 | −40.586 | 1.00 | 56.79 | C |
| ATOM | 3 | CB | SER | B | 23 | −32.349 | −42.985 | −40.618 | 1.00 | 56.78 | C |
| ATOM | 4 | OG | SER | B | 23 | −33.311 | −42.263 | −39.874 | 1.00 | 57.00 | O |
| ATOM | 5 | C | SER | B | 23 | −31.094 | −41.016 | −39.728 | 1.00 | 56.73 | C |
| ATOM | 6 | O | SER | B | 23 | −31.004 | −39.884 | −40.230 | 1.00 | 56.72 | O |
| ATOM | 1 | N | SER | B | 24 | −31.268 | −41.227 | −38.427 | 1.00 | 56.67 | N |
| ATOM | 2 | CA | SER | B | 24 | −31.384 | −40.135 | −37.468 | 1.00 | 56.66 | C |
| ATOM | 3 | CB | SER | B | 24 | −31.630 | −40.691 | −36.065 | 1.00 | 56.64 | C |
| ATOM | 4 | OG | SER | B | 24 | −32.318 | −39.753 | −35.257 | 1.00 | 56.92 | O |
| ATOM | 5 | C | SER | B | 24 | −30.129 | −39.262 | −37.495 | 1.00 | 56.55 | C |
| ATOM | 6 | O | SER | B | 24 | −30.210 | −38.031 | −37.511 | 1.00 | 56.41 | O |
| ATOM | 1 | N | LEU | B | 25 | −28.971 | −39.911 | −37.510 | 1.00 | 56.64 | N |
| ATOM | 2 | CA | LEU | B | 25 | −27.708 | −39.213 | −37.709 | 1.00 | 56.46 | C |
| ATOM | 3 | CB | LEU | B | 25 | −26.567 | −40.216 | −37.867 | 1.00 | 56.37 | C |
| ATOM | 4 | CG | LEU | B | 25 | −25.705 | −40.508 | −36.643 | 1.00 | 56.13 | C |
| ATOM | 5 | CD1 | LEU | B | 25 | −26.483 | −40.290 | −35.370 | 1.00 | 55.71 | C |
| ATOM | 6 | CD2 | LEU | B | 25 | −25.157 | −41.923 | −36.717 | 1.00 | 56.30 | C |
| ATOM | 7 | C | LEU | B | 25 | −27.771 | −38.320 | −38.943 | 1.00 | 56.49 | C |
| ATOM | 8 | O | LEU | B | 25 | −27.389 | −37.148 | −38.894 | 1.00 | 56.42 | O |
| ATOM | 1 | N | LYS | B | 26 | −28.252 | −38.883 | −40.048 | 1.00 | 56.45 | N |
| ATOM | 2 | CA | LYS | B | 26 | −28.317 | −38.164 | −41.308 | 1.00 | 56.29 | C |
| ATOM | 3 | CB | LYS | B | 26 | −28.822 | −39.079 | −42.427 | 1.00 | 56.32 | C |
| ATOM | 4 | CG | LYS | B | 26 | −28.800 | −38.454 | −43.819 | 1.00 | 56.35 | C |
| ATOM | 5 | CD | LYS | B | 26 | −28.778 | −39.516 | −44.917 | 1.00 | 56.35 | C |
| ATOM | 6 | CE | LYS | B | 26 | −30.038 | −40.371 | −44.910 | 1.00 | 56.39 | C |
| ATOM | 7 | NZ | LYS | B | 26 | −29.907 | −41.557 | −45.803 | 1.00 | 56.13 | N |
| ATOM | 8 | C | LYS | B | 26 | −29.190 | −36.924 | −41.168 | 1.00 | 56.32 | C |
| ATOM | 9 | O | LYS | B | 26 | −28.764 | −35.817 | −41.516 | 1.00 | 56.34 | O |
| ATOM | 1 | N | GLU | B | 27 | −30.401 | −37.093 | −40.641 | 1.00 | 56.37 | N |
| ATOM | 2 | CA | GLU | B | 27 | −31.264 | −35.927 | −40.433 | 1.00 | 56.31 | C |
| ATOM | 3 | CB | GLU | B | 27 | −32.635 | −36.332 | −39.894 | 1.00 | 56.40 | C |
| ATOM | 4 | CG | GLU | B | 27 | −33.618 | −36.769 | −40.976 | 1.00 | 56.99 | C |
| ATOM | 5 | CD | GLU | B | 27 | −33.694 | −35.789 | −42.142 | 1.00 | 57.43 | C |
| ATOM | 6 | OE1 | GLU | B | 27 | −32.665 | −35.604 | −42.834 | 1.00 | 57.97 | O |
| ATOM | 7 | OE2 | GLU | B | 27 | −34.785 | −35.218 | −42.379 | 1.00 | 56.59 | O |
| ATOM | 8 | C | GLU | B | 27 | −30.610 | −34.886 | −39.526 | 1.00 | 56.16 | C |
| ATOM | 9 | O | GLU | B | 27 | −30.594 | −33.691 | −39.844 | 1.00 | 55.99 | O |
| ATOM | 1 | N | LEU | B | 28 | −30.063 | −35.352 | −38.405 | 1.00 | 56.19 | N |
| ATOM | 2 | CA | LEU | B | 28 | −29.327 | −34.483 | −37.489 | 1.00 | 56.04 | C |
| ATOM | 3 | CB | LEU | B | 28 | −28.656 | −35.295 | −36.378 | 1.00 | 55.97 | C |
| ATOM | 4 | CG | LEU | B | 28 | −29.364 | −35.366 | −35.022 | 1.00 | 56.13 | C |
| ATOM | 5 | CD1 | LEU | B | 28 | −30.854 | −35.712 | −35.137 | 1.00 | 56.25 | C |
| ATOM | 6 | CD2 | LEU | B | 28 | −28.644 | −36.321 | −34.084 | 1.00 | 56.01 | C |
| ATOM | 7 | C | LEU | B | 28 | −28.287 | −33.671 | −38.241 | 1.00 | 56.00 | C |
| ATOM | 8 | O | LEU | B | 28 | −28.236 | −32.450 | −38.116 | 1.00 | 56.01 | O |
| ATOM | 1 | N | TYR | B | 29 | −27.461 | −34.353 | −39.026 | 1.00 | 55.97 | N |
| ATOM | 2 | CA | TYR | B | 29 | −26.478 | −33.671 | −39.848 | 1.00 | 55.76 | C |
| ATOM | 3 | CB | TYR | B | 29 | −25.722 | −34.659 | −40.731 | 1.00 | 55.53 | C |
| ATOM | 4 | CG | TYR | B | 29 | −24.819 | −33.982 | −41.733 | 1.00 | 55.13 | C |
| ATOM | 5 | CD1 | TYR | B | 29 | −23.669 | −33.320 | −41.323 | 1.00 | 54.84 | C |
| ATOM | 6 | CE1 | TYR | B | 29 | −22.840 | −32.696 | −42.234 | 1.00 | 54.64 | C |
| ATOM | 7 | CZ | TYR | B | 29 | −23.157 | −32.729 | −43.574 | 1.00 | 54.85 | C |
| ATOM | 8 | OH | TYR | B | 29 | −22.339 | −32.111 | −44.487 | 1.00 | 55.02 | O |
| ATOM | 9 | CE2 | TYR | B | 29 | −24.291 | −33.381 | −44.008 | 1.00 | 54.94 | C |
| ATOM | 10 | CD2 | TYR | B | 29 | −25.117 | −34.000 | −43.087 | 1.00 | 54.98 | C |
| ATOM | 11 | C | TYR | B | 29 | −27.140 | −32.618 | −40.720 | 1.00 | 55.90 | C |
| ATOM | 12 | O | TYR | B | 29 | −26.828 | −31.432 | −40.611 | 1.00 | 55.94 | O |
| ATOM | 1 | N | ARG | B | 30 | −28.065 | −33.056 | −41.572 | 1.00 | 56.07 | N |
| ATOM | 2 | CA | ARG | B | 30 | −28.661 | −32.167 | −42.566 | 1.00 | 56.16 | C |
| ATOM | 3 | CB | ARG | B | 30 | −29.655 | −32.915 | −43.458 | 1.00 | 56.13 | C |
| ATOM | 4 | CG | ARG | B | 30 | −30.281 | −32.027 | −44.534 | 1.00 | 56.57 | C |
| ATOM | 5 | CD | ARG | B | 30 | −31.444 | −32.704 | −45.244 | 1.00 | 56.62 | C |
| ATOM | 6 | NE | ARG | B | 30 | −32.497 | −33.102 | −44.313 | 1.00 | 57.56 | N |
| ATOM | 7 | CZ | ARG | B | 30 | −33.525 | −32.334 | −43.968 | 1.00 | 57.96 | C |
| ATOM | 8 | NH1 | ARG | B | 30 | −33.650 | −31.115 | −44.479 | 1.00 | 58.09 | N |
| ATOM | 9 | NH2 | ARG | B | 30 | −34.433 | −32.785 | −43.112 | 1.00 | 58.17 | N |
| ATOM | 10 | C | ARG | B | 30 | −29.336 | −30.945 | −41.958 | 1.00 | 56.07 | C |
| ATOM | 11 | O | ARG | B | 30 | −29.309 | −29.864 | −42.544 | 1.00 | 56.10 | O |
| ATOM | 1 | N | THR | B | 31 | −29.938 | −31.104 | −40.786 | 1.00 | 56.02 | N |
| ATOM | 2 | CA | THR | B | 31 | −30.686 | −29.999 | −40.194 | 1.00 | 55.94 | C |
| ATOM | 3 | CB | THR | B | 31 | −31.968 | −30.495 | −39.510 | 1.00 | 55.94 | C |
| ATOM | 4 | OG1 | THR | B | 31 | −31.627 | −31.427 | −38.476 | 1.00 | 56.01 | O |
| ATOM | 5 | CG2 | THR | B | 31 | −32.879 | −31.172 | −40.522 | 1.00 | 55.91 | C |
| ATOM | 6 | C | THR | B | 31 | −29.878 | −29.166 | −39.196 | 1.00 | 55.97 | C |
| ATOM | 7 | O | THR | B | 31 | −30.158 | −27.982 | −39.000 | 1.00 | 55.91 | O |
| ATOM | 1 | N | LYS | B | 32 | −28.877 | −29.780 | −38.572 | 1.00 | 56.00 | N |
| ATOM | 2 | CA | LYS | B | 32 | −28.160 | −29.137 | −37.469 | 1.00 | 56.02 | C |
| ATOM | 3 | CB | LYS | B | 32 | −28.201 | −30.019 | −36.210 | 1.00 | 56.03 | C |
| ATOM | 4 | CG | LYS | B | 32 | −29.582 | −30.162 | −35.544 | 1.00 | 56.05 | C |
| ATOM | 5 | CD | LYS | B | 32 | −29.920 | −28.956 | −34.665 | 1.00 | 56.16 | C |
| ATOM | 6 | CE | LYS | B | 32 | −31.272 | −29.099 | −33.959 | 1.00 | 55.96 | C |
| ATOM | 7 | NZ | LYS | B | 32 | −31.193 | −29.805 | −32.646 | 1.00 | 55.50 | N |
| ATOM | 8 | C | LYS | B | 32 | −26.711 | −28.750 | −37.796 | 1.00 | 56.03 | C |
| ATOM | 9 | O | LYS | B | 32 | −26.168 | −27.819 | −37.200 | 1.00 | 56.12 | O |
| ATOM | 1 | N | LEU | B | 33 | −26.087 | −29.460 | −38.734 | 1.00 | 55.98 | N |
| ATOM | 2 | CA | LEU | B | 33 | −24.682 | −29.209 | −39.069 | 1.00 | 55.83 | C |
| ATOM | 3 | CB | LEU | B | 33 | −23.881 | −30.508 | −39.031 | 1.00 | 55.81 | C |
| ATOM | 4 | CG | LEU | B | 33 | −22.853 | −30.636 | −37.915 | 1.00 | 55.96 | C |
| ATOM | 5 | CD1 | LEU | B | 33 | −22.175 | −31.991 | −37.990 | 1.00 | 56.34 | C |
| ATOM | 6 | CD2 | LEU | B | 33 | −21.830 | −29.526 | −38.036 | 1.00 | 56.25 | C |
| ATOM | 7 | C | LEU | B | 33 | −24.496 | −28.553 | −40.428 | 1.00 | 55.75 | C |
| ATOM | 8 | O | LEU | B | 33 | −23.834 | −27.521 | −40.551 | 1.00 | 55.70 | O |
| ATOM | 1 | N | LEU | B | 34 | −25.081 | −29.171 | −41.448 | 1.00 | 55.70 | N |
| ATOM | 2 | CA | LEU | B | 34 | −24.952 | −28.699 | −42.817 | 1.00 | 55.64 | C |
| ATOM | 3 | CB | LEU | B | 34 | −25.927 | −29.447 | −43.729 | 1.00 | 55.58 | C |
| ATOM | 4 | CG | LEU | B | 34 | −25.827 | −29.178 | −45.229 | 1.00 | 55.53 | C |
| ATOM | 5 | CD1 | LEU | B | 34 | −24.377 | −29.201 | −45.688 | 1.00 | 55.57 | C |
| ATOM | 6 | CD2 | LEU | B | 34 | −26.656 | −30.193 | −46.001 | 1.00 | 55.60 | C |
| ATOM | 7 | C | LEU | B | 34 | −25.138 | −27.189 | −42.948 | 1.00 | 55.74 | C |
| ATOM | 8 | O | LEU | B | 34 | −24.368 | −26.536 | −43.652 | 1.00 | 55.74 | O |
| ATOM | 1 | N | PRO | B | 35 | −26.158 | −26.624 | −42.272 | 1.00 | 55.81 | N |
| ATOM | 2 | CA | PRO | B | 35 | −26.387 | −25.187 | −42.397 | 1.00 | 55.89 | C |
| ATOM | 3 | CB | PRO | B | 35 | −27.570 | −24.935 | −41.457 | 1.00 | 55.71 | C |
| ATOM | 4 | CG | PRO | B | 35 | −28.242 | −26.244 | −41.333 | 1.00 | 55.71 | C |
| ATOM | 5 | CD | PRO | B | 35 | −27.147 | −27.256 | −41.381 | 1.00 | 55.76 | C |
| ATOM | 6 | C | PRO | B | 35 | −25.183 | −24.372 | −41.948 | 1.00 | 56.06 | C |
| ATOM | 7 | O | PRO | B | 35 | −24.884 | −23.339 | −42.542 | 1.00 | 56.11 | O |
| ATOM | 1 | N | LEU | B | 36 | −24.496 | −24.843 | −40.914 | 1.00 | 56.32 | N |
| ATOM | 2 | CA | LEU | B | 36 | −23.376 | −24.111 | −40.338 | 1.00 | 56.60 | C |
| ATOM | 3 | CB | LEU | B | 36 | −23.087 | −24.654 | −38.947 | 1.00 | 56.61 | C |
| ATOM | 4 | CG | LEU | B | 36 | −22.594 | −23.658 | −37.905 | 1.00 | 56.89 | C |
| ATOM | 5 | CD1 | LEU | B | 36 | −23.153 | −24.031 | −36.552 | 1.00 | 57.25 | C |
| ATOM | 6 | CD2 | LEU | B | 36 | −21.082 | −23.624 | −37.872 | 1.00 | 57.53 | C |
| ATOM | 7 | C | LEU | B | 36 | −22.148 | −24.229 | −41.237 | 1.00 | 56.79 | C |
| ATOM | 8 | O | LEU | B | 36 | −21.443 | −23.237 | −41.507 | 1.00 | 56.95 | O |
| ATOM | 1 | N | GLU | B | 37 | −21.913 | −25.448 | −41.712 | 1.00 | 57.00 | N |
| ATOM | 2 | CA | GLU | B | 37 | −20.879 | −25.705 | −42.703 | 1.00 | 57.34 | C |
| ATOM | 3 | CB | GLU | B | 37 | −20.912 | −27.168 | −43.151 | 1.00 | 57.32 | C |
| ATOM | 4 | CG | GLU | B | 37 | −20.568 | −28.182 | −42.074 | 1.00 | 57.49 | C |
| ATOM | 5 | CD | GLU | B | 37 | −20.509 | −29.602 | −42.617 | 1.00 | 57.48 | C |
| ATOM | 6 | OE1 | GLU | B | 37 | −20.434 | −29.763 | −43.857 | 1.00 | 57.98 | O |
| ATOM | 7 | OE2 | GLU | B | 37 | −20.535 | −30.557 | −41.810 | 1.00 | 57.15 | O |
| ATOM | 8 | C | GLU | B | 37 | −21.050 | −24.800 | −43.925 | 1.00 | 57.50 | C |
| ATOM | 9 | O | GLU | B | 37 | −20.095 | −24.154 | −44.366 | 1.00 | 57.53 | O |
| ATOM | 1 | N | GLU | B | 38 | −22.267 | −24.760 | −44.466 | 1.00 | 57.64 | N |
| ATOM | 2 | CA | GLU | B | 38 | −22.544 | −23.989 | −45.677 | 1.00 | 57.92 | C |
| ATOM | 3 | CB | GLU | B | 38 | −23.939 | −24.302 | −46.222 | 1.00 | 58.10 | C |
| ATOM | 4 | CG | GLU | B | 38 | −24.052 | −25.674 | −46.868 | 1.00 | 59.87 | C |
| ATOM | 5 | CD | GLU | B | 38 | −23.318 | −25.767 | −48.201 | 1.00 | 62.05 | C |
| ATOM | 6 | OE1 | GLU | B | 38 | −23.851 | −25.231 | −49.205 | 1.00 | 62.81 | O |
| ATOM | 7 | OE2 | GLU | B | 38 | −22.219 | −26.384 | −48.245 | 1.00 | 62.50 | O |
| ATOM | 8 | C | GLU | B | 38 | −22.401 | −22.498 | −45.442 | 1.00 | 57.70 | C |
| ATOM | 9 | O | GLU | B | 38 | −21.934 | −21.771 | −46.316 | 1.00 | 57.74 | O |
| ATOM | 1 | N | HIS | B | 39 | −22.798 | −22.045 | −44.259 | 1.00 | 57.50 | N |
| ATOM | 2 | CA | HIS | B | 39 | −22.740 | −20.627 | −43.951 | 1.00 | 57.36 | C |
| ATOM | 3 | CB | HIS | B | 39 | −23.553 | −20.289 | −42.697 | 1.00 | 57.49 | C |
| ATOM | 4 | CG | HIS | B | 39 | −23.914 | −18.837 | −42.582 | 1.00 | 57.86 | C |
| ATOM | 5 | ND1 | HIS | B | 39 | −23.802 | −18.130 | −41.403 | 1.00 | 58.15 | N |
| ATOM | 6 | CE1 | HIS | B | 39 | −24.187 | −16.882 | −41.600 | 1.00 | 57.80 | C |
| ATOM | 7 | NE2 | HIS | B | 39 | −24.540 | −16.751 | −42.866 | 1.00 | 58.12 | N |
| ATOM | 8 | CD2 | HIS | B | 39 | −24.379 | −17.959 | −43.502 | 1.00 | 57.91 | C |
| ATOM | 9 | C | HIS | B | 39 | −21.301 | −20.174 | −43.795 | 1.00 | 57.12 | C |
| ATOM | 10 | O | HIS | B | 39 | −20.915 | −19.146 | −44.341 | 1.00 | 57.14 | O |
| ATOM | 1 | N | TYR | B | 40 | −20.494 | −20.938 | −43.068 | 1.00 | 56.95 | N |
| ATOM | 2 | CA | TYR | B | 40 | −19.110 | −20.504 | −42.885 | 1.00 | 56.80 | C |
| ATOM | 3 | CB | TYR | B | 40 | −18.683 | −20.668 | −41.432 | 1.00 | 56.68 | C |
| ATOM | 4 | CG | TYR | B | 40 | −19.505 | −19.812 | −40.501 | 1.00 | 56.39 | C |
| ATOM | 5 | CD1 | TYR | B | 40 | −19.178 | −18.482 | −40.284 | 1.00 | 56.19 | C |
| ATOM | 6 | CE1 | TYR | B | 40 | −19.929 | −17.692 | −39.445 | 1.00 | 56.13 | C |
| ATOM | 7 | CZ | TYR | B | 40 | −21.032 | −18.227 | −38.811 | 1.00 | 56.27 | C |
| ATOM | 8 | OH | TYR | B | 40 | −21.788 | −17.435 | −37.973 | 1.00 | 56.35 | O |
| ATOM | 9 | CE2 | TYR | B | 40 | −21.384 | −19.546 | −39.017 | 1.00 | 55.86 | C |
| ATOM | 10 | CD2 | TYR | B | 40 | −20.624 | −20.327 | −39.858 | 1.00 | 56.00 | C |
| ATOM | 11 | C | TYR | B | 40 | −18.132 | −21.152 | −43.868 | 1.00 | 56.98 | C |
| ATOM | 12 | O | TYR | B | 40 | −16.916 | −21.131 | −43.664 | 1.00 | 56.80 | O |
| ATOM | 1 | N | ARG | B | 41 | −18.685 | −21.710 | −44.943 | 1.00 | 57.29 | N |
| ATOM | 2 | CA | ARG | B | 41 | −17.902 | −22.199 | −46.082 | 1.00 | 57.67 | C |
| ATOM | 3 | CB | ARG | B | 41 | −17.259 | −21.025 | −46.827 | 1.00 | 57.84 | C |
| ATOM | 4 | CG | ARG | B | 41 | −18.247 | −20.264 | −47.691 | 1.00 | 59.24 | C |
| ATOM | 5 | CD | ARG | B | 41 | −18.706 | −21.140 | −48.847 | 1.00 | 61.74 | C |
| ATOM | 6 | NE | ARG | B | 41 | −20.154 | −21.097 | −49.031 | 1.00 | 63.78 | N |
| ATOM | 7 | CZ | ARG | B | 41 | −20.869 | −22.080 | −49.574 | 1.00 | 65.07 | C |
| ATOM | 8 | NH1 | ARG | B | 41 | −22.184 | −21.953 | −49.699 | 1.00 | 65.94 | N |
| ATOM | 9 | NH2 | ARG | B | 41 | −20.275 | −23.194 | −49.988 | 1.00 | 65.68 | N |
| ATOM | 10 | C | ARG | B | 41 | −16.862 | −23.250 | −45.704 | 1.00 | 57.59 | C |
| ATOM | 11 | O | ARG | B | 41 | −15.745 | −23.266 | −46.227 | 1.00 | 57.63 | O |
| ATOM | 1 | N | PHE | B | 42 | −17.261 | −24.135 | −44.798 | 1.00 | 57.42 | N |
| ATOM | 2 | CA | PHE | B | 42 | −16.440 | −25.247 | −44.347 | 1.00 | 57.25 | C |
| ATOM | 3 | CB | PHE | B | 42 | −17.303 | −26.170 | −43.483 | 1.00 | 57.11 | C |
| ATOM | 4 | CG | PHE | B | 42 | −16.533 | −27.222 | −42.751 | 1.00 | 56.65 | C |
| ATOM | 5 | CD1 | PHE | B | 42 | −16.541 | −28.533 | −43.191 | 1.00 | 56.29 | C |
| ATOM | 6 | CE1 | PHE | B | 42 | −15.840 | −29.506 | −42.518 | 1.00 | 56.21 | C |
| ATOM | 7 | CZ | PHE | B | 42 | −15.123 | −29.177 | −41.385 | 1.00 | 56.63 | C |
| ATOM | 8 | CE2 | PHE | B | 42 | −15.111 | −27.873 | −40.931 | 1.00 | 56.58 | C |
| ATOM | 9 | CD2 | PHE | B | 42 | −15.815 | −26.905 | −41.612 | 1.00 | 56.58 | C |
| ATOM | 10 | C | PHE | B | 42 | −15.866 | −26.027 | −45.525 | 1.00 | 57.37 | C |
| ATOM | 11 | O | PHE | B | 42 | −14.740 | −26.520 | −45.469 | 1.00 | 57.37 | O |
| ATOM | 1 | N | GLY | B | 43 | −16.649 | −26.124 | −46.595 | 1.00 | 57.54 | N |
| ATOM | 2 | CA | GLY | B | 43 | −16.295 | −26.943 | −47.750 | 1.00 | 57.71 | C |
| ATOM | 3 | C | GLY | B | 43 | −15.092 | −26.473 | −48.545 | 1.00 | 57.88 | C |
| ATOM | 4 | O | GLY | B | 43 | −14.552 | −27.222 | −49.358 | 1.00 | 58.01 | O |
| ATOM | 1 | N | SER | B | 44 | −14.671 | −25.234 | −48.320 | 1.00 | 57.99 | N |
| ATOM | 2 | CA | SER | B | 44 | −13.537 | −24.680 | −49.045 | 1.00 | 58.04 | C |
| ATOM | 3 | CB | SER | B | 44 | −13.877 | −23.298 | −49.588 | 1.00 | 57.99 | C |
| ATOM | 4 | OG | SER | B | 44 | −15.092 | −23.328 | −50.315 | 1.00 | 58.35 | O |
| ATOM | 5 | C | SER | B | 44 | −12.324 | −24.589 | −48.144 | 1.00 | 58.15 | C |
| ATOM | 6 | O | SER | B | 44 | −11.338 | −23.930 | −48.482 | 1.00 | 58.23 | O |
| ATOM | 1 | N | PHE | B | 45 | −12.400 | −25.257 | −46.996 | 1.00 | 58.23 | N |
| ATOM | 2 | CA | PHE | B | 45 | −11.345 | −25.183 | −45.995 | 1.00 | 58.33 | C |
| ATOM | 3 | CB | PHE | B | 45 | −11.770 | −24.285 | −44.832 | 1.00 | 58.38 | C |
| ATOM | 4 | CG | PHE | B | 45 | −11.611 | −22.817 | −45.101 | 1.00 | 58.62 | C |
| ATOM | 5 | CD1 | PHE | B | 45 | −10.373 | −22.207 | −44.984 | 1.00 | 58.76 | C |
| ATOM | 6 | CE1 | PHE | B | 45 | −10.225 | −20.851 | −45.226 | 1.00 | 58.85 | C |
| ATOM | 7 | CZ | PHE | B | 45 | −11.322 | −20.087 | −45.582 | 1.00 | 58.65 | C |
| ATOM | 8 | CE2 | PHE | B | 45 | −12.563 | −20.681 | −45.694 | 1.00 | 58.80 | C |
| ATOM | 9 | CD2 | PHE | B | 45 | −12.705 | −22.039 | −45.454 | 1.00 | 58.92 | C |
| ATOM | 10 | C | PHE | B | 45 | −10.947 | −26.549 | −45.451 | 1.00 | 58.40 | C |
| ATOM | 11 | O | PHE | B | 45 | −9.769 | −26.809 | −45.230 | 1.00 | 58.46 | O |
| ATOM | 1 | N | HIS | B | 46 | −11.924 | −27.415 | −45.214 | 1.00 | 58.47 | N |
| ATOM | 2 | CA | HIS | B | 46 | −11.635 | −28.696 | −44.585 | 1.00 | 58.57 | C |
| ATOM | 3 | CB | HIS | B | 46 | −12.053 | −28.677 | −43.118 | 1.00 | 58.52 | C |
| ATOM | 4 | CG | HIS | B | 46 | −11.512 | −27.510 | −42.357 | 1.00 | 58.40 | C |
| ATOM | 5 | ND1 | HIS | B | 46 | −10.256 | −27.510 | −41.790 | 1.00 | 58.69 | N |
| ATOM | 6 | CE1 | HIS | B | 46 | −10.047 | −26.351 | −41.191 | 1.00 | 58.73 | C |
| ATOM | 7 | NE2 | HIS | B | 46 | −11.122 | −25.599 | −41.351 | 1.00 | 58.60 | N |
| ATOM | 8 | CD2 | HIS | B | 46 | −12.053 | −26.301 | −42.077 | 1.00 | 58.47 | C |
| ATOM | 9 | C | HIS | B | 46 | −12.304 | −29.851 | −45.306 | 1.00 | 58.82 | C |
| ATOM | 10 | O | HIS | B | 46 | −11.631 | −30.724 | −45.842 | 1.00 | 58.94 | O |
| ATOM | 1 | N | SER | B | 47 | −13.630 | −29.859 | −45.322 | 1.00 | 59.14 | N |
| ATOM | 2 | CA | SER | B | 47 | −14.356 | −30.936 | −45.976 | 1.00 | 59.57 | C |
| ATOM | 3 | CB | SER | B | 47 | −14.756 | −32.001 | −44.959 | 1.00 | 59.49 | C |
| ATOM | 4 | OG | SER | B | 47 | −13.754 | −32.992 | −44.853 | 1.00 | 59.64 | O |
| ATOM | 5 | C | SER | B | 47 | −15.589 | −30.454 | −46.723 | 1.00 | 59.92 | C |
| ATOM | 6 | O | SER | B | 47 | −16.299 | −29.566 | −46.251 | 1.00 | 60.08 | O |
| ATOM | 1 | N | PRO | B | 48 | −15.848 | −31.043 | −47.899 | 1.00 | 60.20 | N |
| ATOM | 2 | CA | PRO | B | 48 | −17.079 | −30.774 | −48.629 | 1.00 | 60.54 | C |
| ATOM | 3 | CB | PRO | B | 48 | −16.850 | −31.470 | −49.967 | 1.00 | 60.46 | C |
| ATOM | 4 | CG | PRO | B | 48 | −15.873 | −32.544 | −49.664 | 1.00 | 60.43 | C |
| ATOM | 5 | CD | PRO | B | 48 | −14.983 | −32.004 | −48.600 | 1.00 | 60.14 | C |
| ATOM | 6 | C | PRO | B | 48 | −18.249 | −31.409 | −47.892 | 1.00 | 60.95 | C |
| ATOM | 7 | O | PRO | B | 48 | −18.037 | −32.221 | −46.990 | 1.00 | 61.07 | O |
| ATOM | 1 | N | ALA | B | 49 | −19.470 | −31.039 | −48.259 | 1.00 | 61.39 | N |
| ATOM | 2 | CA | ALA | B | 49 | −20.649 | −31.565 | −47.577 | 1.00 | 61.81 | C |
| ATOM | 3 | CB | ALA | B | 49 | −21.887 | −30.779 | −47.964 | 1.00 | 61.89 | C |
| ATOM | 4 | C | ALA | B | 49 | −20.841 | −33.046 | −47.872 | 1.00 | 62.05 | C |
| ATOM | 5 | O | ALA | B | 49 | −20.542 | −33.508 | −48.969 | 1.00 | 61.90 | O |
| ATOM | 1 | N | LEU | B | 50 | −21.346 | −33.778 | −46.884 | 1.00 | 62.63 | N |
| ATOM | 2 | CA | LEU | B | 50 | −21.517 | −35.224 | −46.983 | 1.00 | 63.26 | C |
| ATOM | 3 | CB | LEU | B | 50 | −21.669 | −35.826 | −45.582 | 1.00 | 63.16 | C |
| ATOM | 4 | CG | LEU | B | 50 | −20.604 | −35.424 | −44.553 | 1.00 | 63.07 | C |
| ATOM | 5 | CD1 | LEU | B | 50 | −19.233 | −35.875 | −45.010 | 1.00 | 63.12 | C |
| ATOM | 6 | CD2 | LEU | B | 50 | −20.910 | −35.978 | −43.166 | 1.00 | 62.96 | C |
| ATOM | 7 | C | LEU | B | 50 | −22.713 | −35.607 | −47.863 | 1.00 | 63.95 | C |
| ATOM | 8 | O | LEU | B | 50 | −23.639 | −34.817 | −48.047 | 1.00 | 64.09 | O |
| ATOM | 1 | N | GLU | B | 51 | −22.681 | −36.820 | −48.414 | 1.00 | 64.70 | N |
| ATOM | 2 | CA | GLU | B | 51 | −23.796 | −37.346 | −49.200 | 1.00 | 65.43 | C |
| ATOM | 3 | CB | GLU | B | 51 | −23.280 | −37.991 | −50.482 | 1.00 | 65.54 | C |
| ATOM | 4 | CG | GLU | B | 51 | −22.076 | −37.302 | −51.095 | 1.00 | 66.38 | C |
| ATOM | 5 | CD | GLU | B | 51 | −21.147 | −38.289 | −51.777 | 1.00 | 67.59 | C |
| ATOM | 6 | OE1 | GLU | B | 51 | −20.943 | −39.386 | −51.212 | 1.00 | 68.33 | O |
| ATOM | 7 | OE2 | GLU | B | 51 | −20.620 | −37.976 | −52.870 | 1.00 | 68.07 | O |
| ATOM | 8 | C | GLU | B | 51 | −24.526 | −38.398 | −48.373 | 1.00 | 65.74 | C |
| ATOM | 9 | O | GLU | B | 51 | −24.216 | −38.592 | −47.201 | 1.00 | 65.83 | O |
| ATOM | 1 | N | ASP | B | 52 | −25.489 | −39.086 | −48.977 | 1.00 | 66.11 | N |
| ATOM | 2 | CA | ASP | B | 52 | −26.114 | −40.222 | −48.312 | 1.00 | 66.50 | C |
| ATOM | 3 | CB | ASP | B | 52 | −27.461 | −40.543 | −48.947 | 1.00 | 66.58 | C |
| ATOM | 4 | CG | ASP | B | 52 | −28.508 | −39.495 | −48.641 | 1.00 | 67.19 | C |
| ATOM | 5 | OD1 | ASP | B | 52 | −28.132 | −38.386 | −48.194 | 1.00 | 67.73 | O |
| ATOM | 6 | OD2 | ASP | B | 52 | −29.710 | −39.782 | −48.843 | 1.00 | 67.86 | O |
| ATOM | 7 | C | ASP | B | 52 | −25.187 | −41.420 | −48.418 | 1.00 | 66.69 | C |
| ATOM | 8 | O | ASP | B | 52 | −25.273 | −42.377 | −47.638 | 1.00 | 66.73 | O |
| ATOM | 1 | N | ALA | B | 53 | −24.283 | −41.341 | −49.388 | 1.00 | 66.92 | N |
| ATOM | 2 | CA | ALA | B | 53 | −23.378 | −42.436 | −49.699 | 1.00 | 67.19 | C |
| ATOM | 3 | CB | ALA | B | 53 | −22.599 | −42.128 | −50.969 | 1.00 | 67.25 | C |
| ATOM | 4 | C | ALA | B | 53 | −22.430 | −42.772 | −48.552 | 1.00 | 67.26 | C |
| ATOM | 5 | O | ALA | B | 53 | −22.050 | −43.926 | −48.380 | 1.00 | 67.24 | O |
| ATOM | 1 | N | ASP | B | 54 | −22.043 | −41.771 | −47.770 | 1.00 | 67.45 | N |
| ATOM | 2 | CA | ASP | B | 54 | −21.163 | −42.028 | −46.632 | 1.00 | 67.74 | C |
| ATOM | 3 | CB | ASP | B | 54 | −20.141 | −40.899 | −46.416 | 1.00 | 67.90 | C |
| ATOM | 4 | CG | ASP | B | 54 | −20.661 | −39.544 | −46.847 | 1.00 | 68.67 | C |
| ATOM | 5 | OD1 | ASP | B | 54 | −21.898 | −39.378 | −46.913 | 1.00 | 69.79 | O |
| ATOM | 6 | OD2 | ASP | B | 54 | −19.833 | −38.646 | −47.125 | 1.00 | 69.03 | O |
| ATOM | 7 | C | ASP | B | 54 | −21.958 | −42.329 | −45.363 | 1.00 | 67.62 | C |
| ATOM | 8 | O | ASP | B | 54 | −21.400 | −42.378 | −44.265 | 1.00 | 67.85 | O |
| ATOM | 1 | N | PHE | B | 55 | −23.262 | −42.532 | −45.530 | 1.00 | 67.33 | N |
| ATOM | 2 | CA | PHE | B | 55 | −24.116 | −42.994 | −44.441 | 1.00 | 67.04 | C |
| ATOM | 3 | CB | PHE | B | 55 | −25.306 | −42.056 | −44.244 | 1.00 | 67.01 | C |
| ATOM | 4 | CG | PHE | B | 55 | −24.948 | −40.743 | −43.624 | 1.00 | 66.79 | C |
| ATOM | 5 | CD1 | PHE | B | 55 | −24.578 | −39.670 | −44.412 | 1.00 | 66.59 | C |
| ATOM | 6 | CE1 | PHE | B | 55 | −24.254 | −38.459 | −43.846 | 1.00 | 66.30 | C |
| ATOM | 7 | CZ | PHE | B | 55 | −24.300 | −38.306 | −42.480 | 1.00 | 66.49 | C |
| ATOM | 8 | CE2 | PHE | B | 55 | −24.671 | −39.366 | −41.683 | 1.00 | 66.62 | C |
| ATOM | 9 | CD2 | PHE | B | 55 | −24.995 | −40.576 | −42.255 | 1.00 | 66.65 | C |
| ATOM | 10 | C | PHE | B | 55 | −24.636 | −44.396 | −44.732 | 1.00 | 66.92 | C |
| ATOM | 11 | O | PHE | B | 55 | −24.788 | −45.209 | −43.822 | 1.00 | 66.88 | O |
| ATOM | 1 | N | ASP | B | 56 | −24.915 | −44.669 | −46.004 | 1.00 | 66.74 | N |
| ATOM | 2 | CA | ASP | B | 56 | −25.524 | −45.940 | −46.392 | 1.00 | 66.60 | C |
| ATOM | 3 | CB | ASP | B | 56 | −26.816 | −45.690 | −47.176 | 1.00 | 66.71 | C |
| ATOM | 4 | CG | ASP | B | 56 | −27.844 | −44.905 | −46.379 | 1.00 | 67.09 | C |
| ATOM | 5 | OD1 | ASP | B | 56 | −27.864 | −45.043 | −45.136 | 1.00 | 67.21 | O |
| ATOM | 6 | OD2 | ASP | B | 56 | −28.635 | −44.153 | −46.997 | 1.00 | 67.60 | O |
| ATOM | 7 | C | ASP | B | 56 | −24.591 | −46.835 | −47.209 | 1.00 | 66.36 | C |
| ATOM | 8 | O | ASP | B | 56 | −25.044 | −47.576 | −48.082 | 1.00 | 66.42 | O |
| ATOM | 1 | N | GLY | B | 57 | −23.296 | −46.777 | −46.922 | 1.00 | 66.00 | N |
| ATOM | 2 | CA | GLY | B | 57 | −22.319 | −47.528 | −47.701 | 1.00 | 65.67 | C |
| ATOM | 3 | C | GLY | B | 57 | −21.546 | −48.534 | −46.877 | 1.00 | 65.47 | C |
| ATOM | 4 | O | GLY | B | 57 | −21.455 | −48.408 | −45.656 | 1.00 | 65.50 | O |
| ATOM | 1 | N | LYS | B | 58 | −20.985 | −49.535 | −47.552 | 1.00 | 65.22 | N |
| ATOM | 2 | CA | LYS | B | 58 | −20.241 | −50.602 | −46.883 | 1.00 | 64.95 | C |
| ATOM | 3 | CB | LYS | B | 58 | −19.922 | −51.734 | −47.864 | 1.00 | 64.95 | C |
| ATOM | 4 | CG | LYS | B | 58 | −20.909 | −52.886 | −47.815 | 1.00 | 65.08 | C |
| ATOM | 5 | CD | LYS | B | 58 | −20.919 | −53.661 | −49.120 | 1.00 | 65.47 | C |
| ATOM | 6 | CE | LYS | B | 58 | −19.519 | −54.076 | −49.535 | 1.00 | 65.82 | C |
| ATOM | 7 | NZ | LYS | B | 58 | −19.515 | −54.712 | −50.885 | 1.00 | 66.10 | N |
| ATOM | 8 | C | LYS | B | 58 | −18.958 | −50.095 | −46.235 | 1.00 | 64.68 | C |
| ATOM | 9 | O | LYS | B | 58 | −18.191 | −49.365 | −46.864 | 1.00 | 64.72 | O |
| ATOM | 1 | N | PRO | B | 59 | −18.726 | −50.484 | −44.970 | 1.00 | 64.36 | N |
| ATOM | 2 | CA | PRO | B | 59 | −17.520 | −50.095 | −44.241 | 1.00 | 64.14 | C |
| ATOM | 3 | CB | PRO | B | 59 | −17.587 | −50.937 | −42.956 | 1.00 | 64.07 | C |
| ATOM | 4 | CG | PRO | B | 59 | −18.679 | −51.939 | −43.176 | 1.00 | 64.14 | C |
| ATOM | 5 | CD | PRO | B | 59 | −19.618 | −51.317 | −44.149 | 1.00 | 64.28 | C |
| ATOM | 6 | C | PRO | B | 59 | −16.240 | −50.401 | −45.018 | 1.00 | 63.96 | C |
| ATOM | 7 | O | PRO | B | 59 | −16.143 | −51.438 | −45.678 | 1.00 | 63.99 | O |
| ATOM | 1 | N | MET | B | 60 | −15.277 | −49.488 | −44.941 | 1.00 | 63.71 | N |
| ATOM | 2 | CA | MET | B | 60 | −13.981 | −49.666 | −45.582 | 1.00 | 63.49 | C |
| ATOM | 3 | CB | MET | B | 60 | −13.569 | −48.382 | −46.311 | 1.00 | 63.41 | C |
| ATOM | 4 | CG | MET | B | 60 | −14.477 | −47.957 | −47.455 | 1.00 | 63.33 | C |
| ATOM | 5 | SD | MET | B | 60 | −14.625 | −46.152 | −47.591 | 1.00 | 63.57 | S |
| ATOM | 6 | CE | MET | B | 60 | −15.071 | −45.961 | −49.319 | 1.00 | 63.70 | C |
| ATOM | 7 | C | MET | B | 60 | −12.935 | −49.986 | −44.520 | 1.00 | 63.31 | C |
| ATOM | 8 | O | MET | B | 60 | −13.049 | −49.526 | −43.387 | 1.00 | 63.29 | O |
| ATOM | 1 | N | VAL | B | 61 | −11.928 | −50.779 | −44.871 | 1.00 | 63.07 | N |
| ATOM | 2 | CA | VAL | B | 61 | −10.704 | −50.802 | −44.073 | 1.00 | 62.97 | C |
| ATOM | 3 | CB | VAL | B | 61 | −10.398 | −52.180 | −43.434 | 1.00 | 62.87 | C |
| ATOM | 4 | CG1 | VAL | B | 61 | −11.661 | −52.805 | −42.886 | 1.00 | 62.90 | C |
| ATOM | 5 | CG2 | VAL | B | 61 | −9.741 | −53.104 | −44.425 | 1.00 | 62.98 | C |
| ATOM | 6 | C | VAL | B | 61 | −9.549 | −50.335 | −44.954 | 1.00 | 63.00 | C |
| ATOM | 7 | O | VAL | B | 61 | −9.521 | −50.612 | −46.161 | 1.00 | 63.16 | O |
| ATOM | 1 | N | LEU | B | 62 | −8.615 | −49.599 | −44.360 | 1.00 | 62.78 | N |
| ATOM | 2 | CA | LEU | B | 62 | −7.493 | −49.062 | −45.114 | 1.00 | 62.54 | C |
| ATOM | 3 | CB | LEU | B | 62 | −7.429 | −47.541 | −44.987 | 1.00 | 62.51 | C |
| ATOM | 4 | CG | LEU | B | 62 | −6.260 | −46.869 | −45.715 | 1.00 | 62.61 | C |
| ATOM | 5 | CD1 | LEU | B | 62 | −6.657 | −45.506 | −46.263 | 1.00 | 62.58 | C |
| ATOM | 6 | CD2 | LEU | B | 62 | −5.045 | −46.751 | −44.806 | 1.00 | 62.83 | C |
| ATOM | 7 | C | LEU | B | 62 | −6.184 | −49.675 | −44.665 | 1.00 | 62.41 | C |
| ATOM | 8 | O | LEU | B | 62 | −5.880 | −49.715 | −43.473 | 1.00 | 62.37 | O |
| ATOM | 1 | N | VAL | B | 63 | −5.407 | −50.148 | −45.631 | 1.00 | 62.32 | N |
| ATOM | 2 | CA | VAL | B | 63 | −4.108 | −50.731 | −45.339 | 1.00 | 62.19 | C |
| ATOM | 3 | CB | VAL | B | 63 | −3.968 | −52.128 | −45.956 | 1.00 | 62.15 | C |
| ATOM | 4 | CG1 | VAL | B | 63 | −2.708 | −52.787 | −45.447 | 1.00 | 62.50 | C |
| ATOM | 5 | CG2 | VAL | B | 63 | −5.184 | −52.982 | −45.618 | 1.00 | 61.99 | C |
| ATOM | 6 | C | VAL | B | 63 | −2.981 | −49.830 | −45.837 | 1.00 | 62.07 | C |
| ATOM | 7 | O | VAL | B | 63 | −2.860 | −49.577 | −47.036 | 1.00 | 61.99 | O |
| ATOM | 1 | N | ALA | B | 64 | −2.166 | −49.344 | −44.905 | 1.00 | 62.00 | N |
| ATOM | 2 | CA | ALA | B | 64 | −1.062 | −48.446 | −45.232 | 1.00 | 61.93 | C |
| ATOM | 3 | CB | ALA | B | 64 | −1.355 | −47.043 | −44.742 | 1.00 | 61.86 | C |
| ATOM | 4 | C | ALA | B | 64 | 0.234 | −48.949 | −44.626 | 1.00 | 61.87 | C |
| ATOM | 5 | O | ALA | B | 64 | 0.225 | −49.792 | −43.737 | 1.00 | 62.00 | O |
| ATOM | 1 | N | GLY | B | 65 | 1.347 | −48.418 | −45.106 | 1.00 | 61.82 | N |
| ATOM | 2 | CA | GLY | B | 65 | 2.662 | −48.820 | −44.633 | 1.00 | 62.04 | C |
| ATOM | 3 | C | GLY | B | 65 | 3.663 | −48.413 | −45.689 | 1.00 | 62.30 | C |
| ATOM | 4 | O | GLY | B | 65 | 3.272 | −47.955 | −46.765 | 1.00 | 62.42 | O |
| ATOM | 1 | N | GLN | B | 66 | 4.951 | −48.562 | −45.405 | 1.00 | 62.45 | N |
| ATOM | 2 | CA | GLN | B | 66 | 5.940 | −48.233 | −46.419 | 1.00 | 62.73 | C |
| ATOM | 3 | CB | GLN | B | 66 | 7.199 | −47.616 | −45.816 | 1.00 | 62.81 | C |
| ATOM | 4 | CG | GLN | B | 66 | 7.642 | −48.190 | −44.496 | 1.00 | 63.71 | C |
| ATOM | 5 | CD | GLN | B | 66 | 8.746 | −47.355 | −43.871 | 1.00 | 64.68 | C |
| ATOM | 6 | OE1 | GLN | B | 66 | 9.595 | −46.794 | −44.578 | 1.00 | 65.17 | O |
| ATOM | 7 | NE2 | GLN | B | 66 | 8.740 | −47.264 | −42.543 | 1.00 | 65.07 | N |
| ATOM | 8 | C | GLN | B | 66 | 6.260 | −49.424 | −47.306 | 1.00 | 62.78 | C |
| ATOM | 9 | O | GLN | B | 66 | 5.623 | −50.470 | −47.198 | 1.00 | 62.88 | O |
| ATOM | 1 | N | TYR | B | 67 | 7.237 | −49.258 | −48.189 | 1.00 | 62.88 | N |
| ATOM | 2 | CA | TYR | B | 67 | 7.528 | −50.258 | −49.210 | 1.00 | 62.98 | C |
| ATOM | 3 | CB | TYR | B | 67 | 8.730 | −49.831 | −50.058 | 1.00 | 63.32 | C |
| ATOM | 4 | CG | TYR | B | 67 | 8.484 | −48.619 | −50.930 | 1.00 | 63.88 | C |
| ATOM | 5 | CD1 | TYR | B | 67 | 9.395 | −47.566 | −50.965 | 1.00 | 64.41 | C |
| ATOM | 6 | CE1 | TYR | B | 67 | 9.176 | −46.451 | −51.771 | 1.00 | 64.56 | C |
| ATOM | 7 | CZ | TYR | B | 67 | 8.030 | −46.381 | −52.547 | 1.00 | 64.29 | C |
| ATOM | 8 | OH | TYR | B | 67 | 7.805 | −45.278 | −53.346 | 1.00 | 64.28 | O |
| ATOM | 9 | CE2 | TYR | B | 67 | 7.111 | −47.414 | −52.527 | 1.00 | 64.30 | C |
| ATOM | 10 | CD2 | TYR | B | 67 | 7.340 | −48.526 | −51.721 | 1.00 | 64.33 | C |
| ATOM | 11 | C | TYR | B | 67 | 7.772 | −51.655 | −48.651 | 1.00 | 62.84 | C |
| ATOM | 12 | O | TYR | B | 67 | 8.447 | −51.824 | −47.637 | 1.00 | 62.73 | O |
| ATOM | 1 | N | SER | B | 68 | 7.207 | −52.648 | −49.333 | 1.00 | 62.73 | N |
| ATOM | 2 | CA | SER | B | 68 | 7.460 | −54.060 | −49.053 | 1.00 | 62.58 | C |
| ATOM | 3 | CB | SER | B | 68 | 8.900 | −54.438 | −49.424 | 1.00 | 62.63 | C |
| ATOM | 4 | OG | SER | B | 68 | 9.161 | −54.184 | −50.797 | 1.00 | 62.93 | O |
| ATOM | 5 | C | SER | B | 68 | 7.172 | −54.439 | −47.611 | 1.00 | 62.40 | C |
| ATOM | 6 | O | SER | B | 68 | 7.851 | −55.288 | −47.035 | 1.00 | 62.68 | O |
| ATOM | 1 | N | THR | B | 69 | 6.162 | −53.814 | −47.025 | 1.00 | 61.97 | N |
| ATOM | 2 | CA | THR | B | 69 | 5.803 | −54.129 | −45.658 | 1.00 | 61.63 | C |
| ATOM | 3 | CB | THR | B | 69 | 5.233 | −52.907 | −44.930 | 1.00 | 61.73 | C |
| ATOM | 4 | OG1 | THR | B | 69 | 5.877 | −51.719 | −45.403 | 1.00 | 61.90 | O |
| ATOM | 5 | CG2 | THR | B | 69 | 5.474 | −53.030 | −43.448 | 1.00 | 62.25 | C |
| ATOM | 6 | C | THR | B | 69 | 4.788 | −55.266 | −45.651 | 1.00 | 61.26 | C |
| ATOM | 7 | O | THR | B | 69 | 4.469 | −55.832 | −44.607 | 1.00 | 61.25 | O |
| ATOM | 1 | N | GLY | B | 70 | 4.278 | −55.596 | −46.830 | 1.00 | 60.94 | N |
| ATOM | 2 | CA | GLY | B | 70 | 3.357 | −56.715 | −46.973 | 1.00 | 60.52 | C |
| ATOM | 3 | C | GLY | B | 70 | 1.888 | −56.350 | −46.949 | 1.00 | 60.22 | C |
| ATOM | 4 | O | GLY | B | 70 | 1.065 | −57.156 | −46.549 | 1.00 | 60.21 | O |
| ATOM | 1 | N | LYS | B | 71 | 1.550 | −55.138 | −47.376 | 1.00 | 60.02 | N |
| ATOM | 2 | CA | LYS | B | 71 | 0.156 | −54.712 | −47.405 | 1.00 | 59.94 | C |
| ATOM | 3 | CB | LYS | B | 71 | 0.060 | −53.272 | −47.897 | 1.00 | 59.83 | C |
| ATOM | 4 | CG | LYS | B | 71 | 0.543 | −52.237 | −46.903 | 1.00 | 59.55 | C |
| ATOM | 5 | CD | LYS | B | 71 | 0.862 | −50.922 | −47.596 | 1.00 | 58.81 | C |
| ATOM | 6 | CE | LYS | B | 71 | 2.258 | −50.955 | −48.191 | 1.00 | 58.14 | C |
| ATOM | 7 | NZ | LYS | B | 71 | 2.389 | −50.011 | −49.321 | 1.00 | 57.62 | N |
| ATOM | 8 | C | LYS | B | 71 | −0.670 | −55.620 | −48.313 | 1.00 | 60.06 | C |
| ATOM | 9 | O | LYS | B | 71 | −1.656 | −56.244 | −47.885 | 1.00 | 60.25 | O |
| ATOM | 1 | N | THR | B | 72 | −0.254 | −55.686 | −49.574 | 1.00 | 60.04 | N |
| ATOM | 2 | CA | THR | B | 72 | −0.936 | −56.494 | −50.569 | 1.00 | 60.09 | C |
| ATOM | 3 | CB | THR | B | 72 | −0.198 | −56.451 | −51.928 | 1.00 | 60.10 | C |
| ATOM | 4 | OG1 | THR | B | 72 | 0.276 | −55.121 | −52.180 | 1.00 | 60.02 | O |
| ATOM | 5 | CG2 | THR | B | 72 | −1.126 | −56.878 | −53.059 | 1.00 | 60.05 | C |
| ATOM | 6 | C | THR | B | 72 | −1.048 | −57.931 | −50.069 | 1.00 | 60.14 | C |
| ATOM | 7 | O | THR | B | 72 | −2.105 | −58.562 | −50.181 | 1.00 | 60.40 | O |
| ATOM | 1 | N | SER | B | 73 | 0.043 | −58.438 | −49.501 | 1.00 | 60.12 | N |
| ATOM | 2 | CA | SER | B | 73 | 0.054 | −59.783 | −48.944 | 1.00 | 60.22 | C |
| ATOM | 3 | CB | SER | B | 73 | 1.436 | −60.131 | −48.381 | 1.00 | 60.26 | C |
| ATOM | 4 | OG | SER | B | 73 | 2.380 | −60.336 | −49.421 | 1.00 | 59.98 | O |
| ATOM | 5 | C | SER | B | 73 | −1.001 | −59.901 | −47.856 | 1.00 | 60.19 | C |
| ATOM | 6 | O | SER | B | 73 | −1.798 | −60.825 | −47.841 | 1.00 | 60.56 | O |
| ATOM | 1 | N | PHE | B | 74 | −0.996 | −58.942 | −46.947 | 1.00 | 60.20 | N |
| ATOM | 2 | CA | PHE | B | 74 | −1.956 | −58.885 | −45.867 | 1.00 | 60.14 | C |
| ATOM | 3 | CB | PHE | B | 74 | −1.794 | −57.558 | −45.135 | 1.00 | 60.03 | C |
| ATOM | 4 | CG | PHE | B | 74 | −2.723 | −57.383 | −43.983 | 1.00 | 60.15 | C |
| ATOM | 5 | CD1 | PHE | B | 74 | −2.513 | −58.066 | −42.800 | 1.00 | 60.37 | C |
| ATOM | 6 | CE1 | PHE | B | 74 | −3.366 | −57.899 | −41.736 | 1.00 | 60.41 | C |
| ATOM | 7 | CZ | PHE | B | 74 | −4.437 | −57.033 | −41.843 | 1.00 | 60.50 | C |
| ATOM | 8 | CE2 | PHE | B | 74 | −4.648 | −56.342 | −43.013 | 1.00 | 60.12 | C |
| ATOM | 9 | CD2 | PHE | B | 74 | −3.797 | −56.517 | −44.073 | 1.00 | 60.02 | C |
| ATOM | 10 | C | PHE | B | 74 | −3.376 | −59.038 | −46.397 | 1.00 | 60.19 | C |
| ATOM | 11 | O | PHE | B | 74 | −4.123 | −59.913 | −45.958 | 1.00 | 60.38 | O |
| ATOM | 1 | N | ILE | B | 75 | −3.747 | −58.192 | −47.351 | 1.00 | 60.27 | N |
| ATOM | 2 | CA | ILE | B | 75 | −5.099 | −58.260 | −47.895 | 1.00 | 60.40 | C |
| ATOM | 3 | CB | ILE | B | 75 | −5.362 | −57.131 | −48.894 | 1.00 | 60.26 | C |
| ATOM | 4 | CG1 | ILE | B | 75 | −5.040 | −55.781 | −48.258 | 1.00 | 60.08 | C |
| ATOM | 5 | CD1 | ILE | B | 75 | −5.166 | −54.622 | −49.210 | 1.00 | 59.79 | C |
| ATOM | 6 | CG2 | ILE | B | 75 | −6.805 | −57.159 | −49.353 | 1.00 | 60.22 | C |
| ATOM | 7 | C | ILE | B | 75 | −5.350 | −59.613 | −48.559 | 1.00 | 60.71 | C |
| ATOM | 8 | O | ILE | B | 75 | −6.362 | −60.278 | −48.299 | 1.00 | 61.02 | O |
| ATOM | 1 | N | GLN | B | 76 | −4.415 | −60.007 | −49.419 | 1.00 | 60.91 | N |
| ATOM | 2 | CA | GLN | B | 76 | −4.433 | −61.311 | −50.069 | 1.00 | 61.22 | C |
| ATOM | 3 | CB | GLN | B | 76 | −3.038 | −61.595 | −50.620 | 1.00 | 61.23 | C |
| ATOM | 4 | CG | GLN | B | 76 | −2.935 | −62.776 | −51.553 | 1.00 | 62.03 | C |
| ATOM | 5 | CD | GLN | B | 76 | −2.700 | −62.353 | −52.989 | 1.00 | 63.06 | C |
| ATOM | 6 | OE1 | GLN | B | 76 | −1.909 | −62.972 | −53.707 | 1.00 | 63.47 | O |
| ATOM | 7 | NE2 | GLN | B | 76 | −3.373 | −61.282 | −53.412 | 1.00 | 63.36 | N |
| ATOM | 8 | C | GLN | B | 76 | −4.792 | −62.381 | −49.043 | 1.00 | 61.30 | C |
| ATOM | 9 | O | GLN | B | 76 | −5.596 | −63.280 | −49.294 | 1.00 | 61.68 | O |
| ATOM | 1 | N | TYR | B | 77 | −4.178 | −62.242 | −47.874 | 1.00 | 61.31 | N |
| ATOM | 2 | CA | TYR | B | 77 | −4.286 | −63.181 | −46.768 | 1.00 | 61.46 | C |
| ATOM | 3 | CB | TYR | B | 77 | −3.182 | −62.874 | −45.758 | 1.00 | 61.45 | C |
| ATOM | 4 | CG | TYR | B | 77 | −3.136 | −63.776 | −44.557 | 1.00 | 61.46 | C |
| ATOM | 5 | CD1 | TYR | B | 77 | −3.478 | −63.300 | −43.300 | 1.00 | 61.59 | C |
| ATOM | 6 | CE1 | TYR | B | 77 | −3.419 | −64.117 | −42.185 | 1.00 | 61.89 | C |
| ATOM | 7 | CZ | TYR | B | 77 | −3.012 | −65.428 | −42.324 | 1.00 | 61.81 | C |
| ATOM | 8 | OH | TYR | B | 77 | −2.955 | −66.244 | −41.222 | 1.00 | 61.74 | O |
| ATOM | 9 | CE2 | TYR | B | 77 | −2.663 | −65.923 | −43.565 | 1.00 | 61.90 | C |
| ATOM | 10 | CD2 | TYR | B | 77 | −2.727 | −65.097 | −44.671 | 1.00 | 61.71 | C |
| ATOM | 11 | C | TYR | B | 77 | −5.645 | −63.093 | −46.092 | 1.00 | 61.48 | C |
| ATOM | 12 | O | TYR | B | 77 | −6.213 | −64.112 | −45.699 | 1.00 | 61.51 | O |
| ATOM | 1 | N | LEU | B | 78 | −6.159 | −61.874 | −45.947 | 1.00 | 61.31 | N |
| ATOM | 2 | CA | LEU | B | 78 | −7.491 | −61.687 | −45.390 | 1.00 | 61.65 | C |
| ATOM | 3 | CB | LEU | B | 78 | −7.806 | −60.206 | −45.219 | 1.00 | 61.70 | C |
| ATOM | 4 | CG | LEU | B | 78 | −7.478 | −59.629 | −43.844 | 1.00 | 62.28 | C |
| ATOM | 5 | CD1 | LEU | B | 78 | −7.621 | −58.112 | −43.859 | 1.00 | 62.88 | C |
| ATOM | 6 | CD2 | LEU | B | 78 | −8.408 | −60.242 | −42.813 | 1.00 | 62.72 | C |
| ATOM | 7 | C | LEU | B | 78 | −8.531 | −62.325 | −46.287 | 1.00 | 61.78 | C |
| ATOM | 8 | O | LEU | B | 78 | −9.488 | −62.939 | −45.814 | 1.00 | 61.79 | O |
| ATOM | 1 | N | LEU | B | 79 | −8.337 | −62.181 | −47.591 | 1.00 | 61.84 | N |
| ATOM | 2 | CA | LEU | B | 79 | −9.298 | −62.702 | −48.542 | 1.00 | 62.22 | C |
| ATOM | 3 | CB | LEU | B | 79 | −9.254 | −61.888 | −49.832 | 1.00 | 62.29 | C |
| ATOM | 4 | CG | LEU | B | 79 | −9.512 | −60.386 | −49.707 | 1.00 | 62.42 | C |
| ATOM | 5 | CD1 | LEU | B | 79 | −9.514 | −59.736 | −51.083 | 1.00 | 62.58 | C |
| ATOM | 6 | CD2 | LEU | B | 79 | −10.824 | −60.120 | −48.987 | 1.00 | 62.69 | C |
| ATOM | 7 | C | LEU | B | 79 | −9.047 | −64.168 | −48.851 | 1.00 | 62.47 | C |
| ATOM | 8 | O | LEU | B | 79 | −9.897 | −64.833 | −49.444 | 1.00 | 62.39 | O |
| ATOM | 1 | N | GLU | B | 80 | −7.883 | −64.668 | −48.443 | 1.00 | 62.76 | N |
| ATOM | 2 | CA | GLU | B | 80 | −7.442 | −66.001 | −48.849 | 1.00 | 63.58 | C |
| ATOM | 3 | CB | GLU | B | 80 | −8.137 | −67.091 | −48.031 | 1.00 | 63.72 | C |
| ATOM | 4 | CG | GLU | B | 80 | −7.551 | −67.293 | −46.633 | 1.00 | 65.58 | C |
| ATOM | 5 | CD | GLU | B | 80 | −6.542 | −68.443 | −46.559 | 1.00 | 68.13 | C |
| ATOM | 6 | OE1 | GLU | B | 80 | −6.912 | −69.522 | −46.016 | 1.00 | 69.12 | O |
| ATOM | 7 | OE2 | GLU | B | 80 | −5.388 | −68.278 | −47.045 | 1.00 | 68.88 | O |
| ATOM | 8 | C | GLU | B | 80 | −7.714 | −66.180 | −50.339 | 1.00 | 63.57 | C |
| ATOM | 9 | O | GLU | B | 80 | −8.206 | −67.219 | −50.777 | 1.00 | 63.47 | O |
| ATOM | 1 | N | GLN | B | 81 | −7.399 | −65.133 | −51.099 | 1.00 | 63.78 | N |
| ATOM | 2 | CA | GLN | B | 81 | −7.586 | −65.106 | −52.541 | 1.00 | 63.93 | C |
| ATOM | 3 | CB | GLN | B | 81 | −9.062 | −64.900 | −52.890 | 1.00 | 63.84 | C |
| ATOM | 4 | CG | GLN | B | 81 | −9.347 | −64.947 | −54.387 | 1.00 | 63.39 | C |
| ATOM | 5 | CD | GLN | B | 81 | −10.824 | −64.864 | −54.707 | 1.00 | 62.75 | C |
| ATOM | 6 | OE1 | GLN | B | 81 | −11.239 | −65.199 | −55.812 | 1.00 | 63.13 | O |
| ATOM | 7 | NE2 | GLN | B | 81 | −11.627 | −64.417 | −53.746 | 1.00 | 62.73 | N |
| ATOM | 8 | C | GLN | B | 81 | −6.739 | −63.987 | −53.152 | 1.00 | 64.17 | C |
| ATOM | 9 | O | GLN | B | 81 | −6.526 | −62.952 | −52.522 | 1.00 | 64.15 | O |
| ATOM | 1 | N | GLU | B | 82 | −6.257 | −64.204 | −54.375 | 1.00 | 64.47 | N |
| ATOM | 2 | CA | GLU | B | 82 | −5.416 | −63.227 | −55.065 | 1.00 | 64.72 | C |
| ATOM | 3 | CB | GLU | B | 82 | −4.583 | −63.910 | −56.160 | 1.00 | 64.74 | C |
| ATOM | 4 | CG | GLU | B | 82 | −3.346 | −63.123 | −56.595 | 1.00 | 65.36 | C |
| ATOM | 5 | CD | GLU | B | 82 | −2.097 | −63.996 | −56.753 | 1.00 | 66.14 | C |
| ATOM | 6 | OE1 | GLU | B | 82 | −2.185 | −65.080 | −57.373 | 1.00 | 66.18 | O |
| ATOM | 7 | OE2 | GLU | B | 82 | −1.020 | −63.591 | −56.255 | 1.00 | 66.28 | O |
| ATOM | 8 | C | GLU | B | 82 | −6.232 | −62.057 | −55.631 | 1.00 | 64.81 | C |
| ATOM | 9 | O | GLU | B | 82 | −7.363 | −62.233 | −56.085 | 1.00 | 64.77 | O |
| ATOM | 1 | N | VAL | B | 83 | −5.652 | −60.862 | −55.588 | 1.00 | 64.99 | N |
| ATOM | 2 | CA | VAL | B | 83 | −6.325 | −59.654 | −56.052 | 1.00 | 65.07 | C |
| ATOM | 3 | CB | VAL | B | 83 | −5.794 | −58.413 | −55.322 | 1.00 | 65.06 | C |
| ATOM | 4 | CG1 | VAL | B | 83 | −6.742 | −57.244 | −55.511 | 1.00 | 65.06 | C |
| ATOM | 5 | CG2 | VAL | B | 83 | −5.598 | −58.712 | −53.847 | 1.00 | 64.87 | C |
| ATOM | 6 | C | VAL | B | 83 | −6.138 | −59.465 | −57.556 | 1.00 | 65.23 | C |
| ATOM | 7 | O | VAL | B | 83 | −5.009 | −59.404 | −58.040 | 1.00 | 65.10 | O |
| ATOM | 1 | N | PRO | B | 84 | −7.252 | −59.365 | −58.297 | 1.00 | 65.51 | N |
| ATOM | 2 | CA | PRO | B | 84 | −7.253 | −59.247 | −59.752 | 1.00 | 65.77 | C |
| ATOM | 3 | CB | PRO | B | 84 | −8.656 | −58.723 | −60.051 | 1.00 | 65.66 | C |
| ATOM | 4 | CG | PRO | B | 84 | −9.490 | −59.315 | −58.983 | 1.00 | 65.56 | C |
| ATOM | 5 | CD | PRO | B | 84 | −8.620 | −59.365 | −57.750 | 1.00 | 65.53 | C |
| ATOM | 6 | C | PRO | B | 84 | −6.206 | −58.275 | −60.284 | 1.00 | 66.15 | C |
| ATOM | 7 | O | PRO | B | 84 | −5.487 | −58.603 | −61.225 | 1.00 | 66.22 | O |
| ATOM | 1 | N | GLY | B | 85 | −6.116 | −57.094 | −59.685 | 1.00 | 66.61 | N |
| ATOM | 2 | CA | GLY | B | 85 | −5.194 | −56.072 | −60.165 | 1.00 | 67.21 | C |
| ATOM | 3 | C | GLY | B | 85 | −3.847 | −56.044 | −59.464 | 1.00 | 67.67 | C |
| ATOM | 4 | O | GLY | B | 85 | −2.820 | −55.785 | −60.094 | 1.00 | 67.61 | O |
| ATOM | 1 | N | SER | B | 86 | −3.848 | −56.313 | −58.161 | 1.00 | 68.17 | N |
| ATOM | 2 | CA | SER | B | 86 | −2.644 | −56.150 | −57.339 | 1.00 | 68.71 | C |
| ATOM | 3 | CB | SER | B | 86 | −3.014 | −55.711 | −55.914 | 1.00 | 68.74 | C |
| ATOM | 4 | OG | SER | B | 86 | −3.020 | −54.297 | −55.787 | 1.00 | 68.90 | O |
| ATOM | 5 | C | SER | B | 86 | −1.725 | −57.374 | −57.281 | 1.00 | 69.01 | C |
| ATOM | 6 | O | SER | B | 86 | −2.054 | −58.386 | −56.652 | 1.00 | 69.11 | O |
| ATOM | 1 | N | ARG | B | 87 | −0.565 | −57.263 | −57.927 | 1.00 | 69.32 | N |
| ATOM | 2 | CA | ARG | B | 87 | 0.485 | −58.266 | −57.801 | 1.00 | 69.63 | C |
| ATOM | 3 | CB | ARG | B | 87 | 1.378 | −58.274 | −59.044 | 1.00 | 69.53 | C |
| ATOM | 4 | CG | ARG | B | 87 | 0.269 | −59.653 | −59.740 | 0.00 | 50.00 | C |
| ATOM | 5 | CD | ARG | B | 87 | 0.975 | −60.115 | −60.993 | 0.00 | 50.00 | C |
| ATOM | 6 | NE | ARG | B | 87 | 1.731 | −61.343 | −60.776 | 0.00 | 50.00 | N |
| ATOM | 7 | CZ | ARG | B | 87 | 1.237 | −62.572 | −60.852 | 0.00 | 50.00 | C |
| ATOM | 8 | NH1 | ARG | B | 87 | 2.033 | −63.609 | −60.623 | 0.00 | 50.00 | N |
| ATOM | 9 | NH2 | ARG | B | 87 | −0.028 | −62.779 | −61.180 | 0.00 | 50.00 | N |
| ATOM | 10 | C | ARG | B | 87 | 1.319 | −57.999 | −56.544 | 1.00 | 69.92 | C |
| ATOM | 11 | O | ARG | B | 87 | 1.025 | −57.085 | −55.771 | 1.00 | 69.93 | O |
| ATOM | 1 | N | VAL | B | 88 | 2.357 | −58.803 | −56.344 | 1.00 | 70.32 | N |
| ATOM | 2 | CA | VAL | B | 88 | 3.222 | −58.678 | −55.172 | 1.00 | 70.65 | C |
| ATOM | 3 | CB | VAL | B | 88 | 2.715 | −59.565 | −54.007 | 1.00 | 70.63 | C |
| ATOM | 4 | CG1 | VAL | B | 88 | 3.838 | −59.875 | −53.028 | 1.00 | 70.52 | C |
| ATOM | 5 | CG2 | VAL | B | 88 | 2.102 | −60.855 | −54.544 | 1.00 | 70.76 | C |
| ATOM | 6 | C | VAL | B | 88 | 4.664 | −59.035 | −55.538 | 1.00 | 70.97 | C |
| ATOM | 7 | O | VAL | B | 88 | 4.906 | −59.997 | −56.272 | 1.00 | 71.03 | O |
| ATOM | 1 | N | GLY | B | 89 | 5.619 | −58.255 | −55.040 | 1.00 | 71.28 | N |
| ATOM | 2 | CA | GLY | B | 89 | 7.020 | −58.471 | −55.389 | 1.00 | 71.78 | C |
| ATOM | 3 | C | GLY | B | 89 | 8.015 | −57.882 | −54.410 | 1.00 | 72.17 | C |
| ATOM | 4 | O | GLY | B | 89 | 7.627 | −57.243 | −53.432 | 1.00 | 72.08 | O |
| ATOM | 1 | N | PRO | B | 90 | 9.316 | −58.087 | −54.681 | 1.00 | 72.59 | N |
| ATOM | 2 | CA | PRO | B | 90 | 10.417 | −57.648 | −53.818 | 1.00 | 72.92 | C |
| ATOM | 3 | CB | PRO | B | 90 | 11.655 | −58.251 | −54.493 | 1.00 | 72.90 | C |
| ATOM | 4 | CG | PRO | B | 90 | 11.259 | −58.441 | −55.924 | 1.00 | 72.85 | C |
| ATOM | 5 | CD | PRO | B | 90 | 9.797 | −58.771 | −55.896 | 1.00 | 72.65 | C |
| ATOM | 6 | C | PRO | B | 90 | 10.523 | −56.126 | −53.765 | 1.00 | 73.27 | C |
| ATOM | 7 | O | PRO | B | 90 | 9.958 | −55.499 | −52.866 | 1.00 | 73.27 | O |
| ATOM | 1 | N | GLU | B | 91 | 11.246 | −55.546 | −54.721 | 1.00 | 73.71 | N |
| ATOM | 2 | CA | GLU | B | 91 | 11.333 | −54.094 | −54.859 | 1.00 | 74.09 | C |
| ATOM | 3 | CB | GLU | B | 91 | 12.405 | −53.714 | −55.891 | 1.00 | 74.13 | C |
| ATOM | 4 | CG | GLU | B | 91 | 13.840 | −53.941 | −55.421 | 1.00 | 74.32 | C |
| ATOM | 5 | CD | GLU | B | 91 | 14.010 | −53.220 | −53.305 | 0.00 | 50.00 | C |
| ATOM | 6 | OE1 | GLU | B | 91 | 15.017 | −53.953 | −53.217 | 0.00 | 50.00 | O |
| ATOM | 7 | OE2 | GLU | B | 91 | 13.312 | −52.915 | −52.308 | 0.00 | 50.00 | O |
| ATOM | 8 | C | GLU | B | 91 | 9.971 | −53.526 | −55.270 | 1.00 | 74.25 | C |
| ATOM | 9 | O | GLU | B | 91 | 9.245 | −54.168 | −56.034 | 1.00 | 74.34 | O |
| ATOM | 1 | N | PRO | B | 92 | 9.626 | −52.323 | −54.759 | 1.00 | 74.34 | N |
| ATOM | 2 | CA | PRO | B | 92 | 8.362 | −51.615 | −54.984 | 1.00 | 74.34 | C |
| ATOM | 3 | CB | PRO | B | 92 | 8.766 | −50.133 | −54.870 | 1.00 | 74.35 | C |
| ATOM | 4 | CG | PRO | B | 92 | 10.183 | −50.122 | −54.284 | 1.00 | 74.33 | C |
| ATOM | 5 | CD | PRO | B | 92 | 10.513 | −51.535 | −53.889 | 1.00 | 74.39 | C |
| ATOM | 6 | C | PRO | B | 92 | 7.728 | −51.884 | −56.354 | 1.00 | 74.33 | C |
| ATOM | 7 | O | PRO | B | 92 | 8.391 | −51.737 | −57.391 | 1.00 | 74.31 | O |
| ATOM | 1 | N | THR | B | 93 | 6.448 | −52.260 | −56.348 | 1.00 | 74.15 | N |
| ATOM | 2 | CA | THR | B | 93 | 5.746 | −52.645 | −57.578 | 1.00 | 73.95 | C |
| ATOM | 3 | CB | THR | B | 93 | 5.311 | −54.133 | −57.540 | 1.00 | 74.06 | C |
| ATOM | 4 | OG1 | THR | B | 93 | 4.909 | −54.490 | −56.194 | 1.00 | 73.87 | O |
| ATOM | 5 | CG2 | THR | B | 93 | 6.469 | −55.058 | −57.997 | 1.00 | 74.23 | C |
| ATOM | 6 | C | THR | B | 93 | 4.532 | −51.770 | −57.907 | 1.00 | 73.59 | C |
| ATOM | 7 | O | THR | B | 93 | 4.528 | −51.057 | −58.917 | 1.00 | 73.58 | O |
| ATOM | 1 | N | THR | B | 94 | 3.507 | −51.839 | −57.055 | 1.00 | 73.13 | N |
| ATOM | 2 | CA | THR | B | 94 | 2.259 | −51.097 | −57.264 | 1.00 | 72.66 | C |
| ATOM | 3 | CB | THR | B | 94 | 1.120 | −51.644 | −56.357 | 1.00 | 72.80 | C |
| ATOM | 4 | OG1 | THR | B | 94 | 1.526 | −51.598 | −54.978 | 1.00 | 72.65 | O |
| ATOM | 5 | CG2 | THR | B | 94 | 0.780 | −53.088 | −56.729 | 1.00 | 72.95 | C |
| ATOM | 6 | C | THR | B | 94 | 2.442 | −49.593 | −57.020 | 1.00 | 72.18 | C |
| ATOM | 7 | O | THR | B | 94 | 3.059 | −49.188 | −56.033 | 1.00 | 72.21 | O |
| ATOM | 1 | N | ASP | B | 95 | 1.900 | −48.767 | −57.914 | 1.00 | 71.45 | N |
| ATOM | 2 | CA | ASP | B | 95 | 2.119 | −47.320 | −57.839 | 1.00 | 70.73 | C |
| ATOM | 3 | CB | ASP | B | 95 | 2.915 | −46.839 | −59.063 | 1.00 | 70.79 | C |
| ATOM | 4 | CG | ASP | B | 95 | 3.164 | −46.932 | −59.646 | 0.00 | 50.00 | C |
| ATOM | 5 | OD1 | ASP | B | 95 | 2.550 | −47.842 | −60.280 | 0.00 | 50.00 | O |
| ATOM | 6 | OD2 | ASP | B | 95 | 3.842 | −46.098 | −60.301 | 0.00 | 50.00 | O |
| ATOM | 7 | C | ASP | B | 95 | 0.835 | −46.498 | −57.697 | 1.00 | 70.16 | C |
| ATOM | 8 | O | ASP | B | 95 | 0.762 | −45.367 | −58.187 | 1.00 | 70.07 | O |
| ATOM | 1 | N | CYS | B | 96 | −0.165 | −47.052 | −57.014 | 1.00 | 69.46 | N |
| ATOM | 2 | CA | CYS | B | 96 | −1.433 | −46.343 | −56.840 | 1.00 | 68.81 | C |
| ATOM | 3 | CB | CYS | B | 96 | −2.198 | −46.296 | −58.165 | 1.00 | 68.91 | C |
| ATOM | 4 | SG | CYS | B | 96 | −2.997 | −47.853 | −58.603 | 1.00 | 69.45 | S |
| ATOM | 5 | C | CYS | B | 96 | −2.334 | −46.935 | −55.752 | 1.00 | 68.20 | C |
| ATOM | 6 | O | CYS | B | 96 | −1.935 | −47.834 | −55.013 | 1.00 | 68.12 | O |
| ATOM | 1 | N | PHE | B | 97 | −3.553 | −46.408 | −55.668 | 1.00 | 67.48 | N |
| ATOM | 2 | CA | PHE | B | 97 | −4.549 | −46.870 | −54.713 | 1.00 | 66.69 | C |
| ATOM | 3 | CB | PHE | B | 97 | −5.414 | −45.706 | −54.228 | 1.00 | 66.64 | C |
| ATOM | 4 | CG | PHE | B | 97 | −4.672 | −44.689 | −53.416 | 1.00 | 66.32 | C |
| ATOM | 5 | CD1 | PHE | B | 97 | −3.822 | −43.780 | −54.025 | 1.00 | 66.08 | C |
| ATOM | 6 | CE1 | PHE | B | 97 | −3.140 | −42.841 | −53.281 | 1.00 | 65.88 | C |
| ATOM | 7 | CZ | PHE | B | 97 | −3.311 | −42.793 | −51.915 | 1.00 | 66.18 | C |
| ATOM | 8 | CE2 | PHE | B | 97 | −4.162 | −43.688 | −51.295 | 1.00 | 66.18 | C |
| ATOM | 9 | CD2 | PHE | B | 97 | −4.839 | −44.627 | −52.045 | 1.00 | 66.18 | C |
| ATOM | 10 | C | PHE | B | 97 | −5.450 | −47.902 | −55.365 | 1.00 | 66.32 | C |
| ATOM | 11 | O | PHE | B | 97 | −6.032 | −47.657 | −56.421 | 1.00 | 66.17 | O |
| ATOM | 1 | N | VAL | B | 98 | −5.568 | −49.056 | −54.726 | 1.00 | 65.88 | N |
| ATOM | 2 | CA | VAL | B | 98 | −6.434 | −50.108 | −55.218 | 1.00 | 65.43 | C |
| ATOM | 3 | CB | VAL | B | 98 | −5.678 | −51.438 | −55.357 | 1.00 | 65.36 | C |
| ATOM | 4 | CG1 | VAL | B | 98 | −6.570 | −52.488 | −55.991 | 1.00 | 65.18 | C |
| ATOM | 5 | CG2 | VAL | B | 98 | −4.413 | −51.243 | −56.171 | 1.00 | 65.13 | C |
| ATOM | 6 | C | VAL | B | 98 | −7.590 | −50.287 | −54.251 | 1.00 | 65.26 | C |
| ATOM | 7 | O | VAL | B | 98 | −7.389 | −50.620 | −53.086 | 1.00 | 65.27 | O |
| ATOM | 1 | N | ALA | B | 99 | −8.801 | −50.050 | −54.732 | 1.00 | 65.07 | N |
| ATOM | 2 | CA | ALA | B | 99 | −9.978 | −50.225 | −53.905 | 1.00 | 65.02 | C |
| ATOM | 3 | CB | ALA | B | 99 | −11.021 | −49.179 | −54.242 | 1.00 | 65.00 | C |
| ATOM | 4 | C | ALA | B | 99 | −10.539 | −51.619 | −54.108 | 1.00 | 65.03 | C |
| ATOM | 5 | O | ALA | B | 99 | −11.338 | −51.847 | −55.012 | 1.00 | 65.09 | O |
| ATOM | 1 | N | VAL | B | 100 | −10.109 | −52.555 | −53.274 | 1.00 | 65.12 | N |
| ATOM | 2 | CA | VAL | B | 100 | −10.605 | −53.922 | −53.363 | 1.00 | 65.24 | C |
| ATOM | 3 | CB | VAL | B | 100 | −9.693 | −54.914 | −52.616 | 1.00 | 65.20 | C |
| ATOM | 4 | CG1 | VAL | B | 100 | −8.233 | −54.672 | −52.969 | 1.00 | 65.10 | C |
| ATOM | 5 | CG2 | VAL | B | 100 | −10.100 | −56.345 | −52.931 | 1.00 | 65.15 | C |
| ATOM | 6 | C | VAL | B | 100 | −12.012 | −53.995 | −52.786 | 1.00 | 65.39 | C |
| ATOM | 7 | O | VAL | B | 100 | −12.199 | −53.938 | −51.570 | 1.00 | 65.48 | O |
| ATOM | 1 | N | MET | B | 101 | −13.001 | −54.107 | −53.665 | 1.00 | 65.56 | N |
| ATOM | 2 | CA | MET | B | 101 | −14.394 | −54.161 | −53.245 | 1.00 | 65.78 | C |
| ATOM | 3 | CB | MET | B | 101 | −15.100 | −52.850 | −53.572 | 1.00 | 65.79 | C |
| ATOM | 4 | CG | MET | B | 101 | −14.752 | −52.294 | −54.941 | 1.00 | 65.81 | C |
| ATOM | 5 | SD | MET | B | 101 | −15.844 | −50.959 | −55.463 | 1.00 | 65.65 | S |
| ATOM | 6 | CE | MET | B | 101 | −15.750 | −49.849 | −54.058 | 1.00 | 65.57 | C |
| ATOM | 7 | C | MET | B | 101 | −15.110 | −55.317 | −53.917 | 1.00 | 66.02 | C |
| ATOM | 8 | O | MET | B | 101 | −14.552 | −55.978 | −54.790 | 1.00 | 66.05 | O |
| ATOM | 1 | N | HIS | B | 102 | −16.350 | −55.559 | −53.514 | 1.00 | 66.39 | N |
| ATOM | 2 | CA | HIS | B | 102 | −17.107 | −56.667 | −54.073 | 1.00 | 66.80 | C |
| ATOM | 3 | CB | HIS | B | 102 | −18.418 | −56.881 | −53.314 | 1.00 | 66.73 | C |
| ATOM | 4 | CG | HIS | B | 102 | −19.129 | −58.142 | −53.694 | 1.00 | 66.79 | C |
| ATOM | 5 | ND1 | HIS | B | 102 | −18.664 | −59.392 | −53.343 | 1.00 | 66.68 | N |
| ATOM | 6 | CE1 | HIS | B | 102 | −19.483 | −60.313 | −53.819 | 1.00 | 66.49 | C |
| ATOM | 7 | NE2 | HIS | B | 102 | −20.461 | −59.706 | −54.469 | 1.00 | 66.63 | N |
| ATOM | 8 | CD2 | HIS | B | 102 | −20.262 | −58.347 | −54.408 | 1.00 | 66.78 | C |
| ATOM | 9 | C | HIS | B | 102 | −17.373 | −56.459 | −55.561 | 1.00 | 67.13 | C |
| ATOM | 10 | O | HIS | B | 102 | −16.949 | −55.465 | −56.145 | 1.00 | 67.23 | O |
| ATOM | 1 | N | GLY | B | 103 | −18.072 | −57.408 | −56.168 | 1.00 | 67.46 | N |
| ATOM | 2 | CA | GLY | B | 103 | −18.379 | −57.341 | −57.583 | 1.00 | 67.99 | C |
| ATOM | 3 | C | GLY | B | 103 | −18.712 | −58.729 | −58.069 | 1.00 | 68.39 | C |
| ATOM | 4 | O | GLY | B | 103 | −18.227 | −59.715 | −57.519 | 1.00 | 68.41 | O |
| ATOM | 1 | N | GLU | B | 104 | −19.545 | −58.812 | −59.099 | 1.00 | 68.82 | N |
| ATOM | 2 | CA | GLU | B | 104 | −19.993 | −60.105 | −59.591 | 1.00 | 69.31 | C |
| ATOM | 3 | CB | GLU | B | 104 | −21.329 | −59.968 | −60.333 | 1.00 | 69.45 | C |
| ATOM | 4 | CG | GLU | B | 104 | −21.933 | −61.291 | −60.806 | 1.00 | 70.31 | C |
| ATOM | 5 | CD | GLU | B | 104 | −21.826 | −62.411 | −59.768 | 1.00 | 71.40 | C |
| ATOM | 6 | OE1 | GLU | B | 104 | −21.442 | −62.136 | −58.606 | 1.00 | 71.64 | O |
| ATOM | 7 | OE2 | GLU | B | 104 | −22.124 | −63.575 | −60.123 | 1.00 | 71.78 | O |
| ATOM | 8 | C | GLU | B | 104 | −18.934 | −60.790 | −60.460 | 1.00 | 69.42 | C |
| ATOM | 9 | O | GLU | B | 104 | −18.745 | −62.007 | −60.384 | 1.00 | 69.49 | O |
| ATOM | 1 | N | THR | B | 105 | −18.237 | −60.003 | −61.272 | 1.00 | 69.53 | N |
| ATOM | 2 | CA | THR | B | 105 | −17.191 | −60.542 | −62.131 | 1.00 | 69.56 | C |
| ATOM | 3 | CB | THR | B | 105 | −17.345 | −60.049 | −63.590 | 1.00 | 69.61 | C |
| ATOM | 4 | OG1 | THR | B | 105 | −16.517 | −60.837 | −64.455 | 1.00 | 69.65 | O |
| ATOM | 5 | CG2 | THR | B | 105 | −16.957 | −58.573 | −63.713 | 1.00 | 69.72 | C |
| ATOM | 6 | C | THR | B | 105 | −15.805 | −60.172 | −61.606 | 1.00 | 69.52 | C |
| ATOM | 7 | O | THR | B | 105 | −15.611 | −59.099 | −61.030 | 1.00 | 69.53 | O |
| ATOM | 1 | N | GLU | B | 106 | −14.849 | −61.075 | −61.792 | 1.00 | 69.46 | N |
| ATOM | 2 | CA | GLU | B | 106 | −13.460 | −60.778 | −61.477 | 1.00 | 69.46 | C |
| ATOM | 3 | CB | GLU | B | 106 | −12.637 | −62.066 | −61.400 | 1.00 | 69.51 | C |
| ATOM | 4 | CG | GLU | B | 106 | −12.955 | −62.949 | −60.198 | 1.00 | 69.53 | C |
| ATOM | 5 | CD | GLU | B | 106 | −12.289 | −64.318 | −60.277 | 1.00 | 69.39 | C |
| ATOM | 6 | OE1 | GLU | B | 106 | −11.990 | −64.754 | −61.456 | 1.00 | 69.37 | O |
| ATOM | 7 | OE2 | GLU | B | 106 | −12.062 | −64.963 | −59.165 | 1.00 | 68.93 | O |
| ATOM | 8 | C | GLU | B | 106 | −12.899 | −59.863 | −62.557 | 1.00 | 69.46 | C |
| ATOM | 9 | O | GLU | B | 106 | −13.145 | −60.073 | −63.743 | 1.00 | 69.46 | O |
| ATOM | 1 | N | GLY | B | 107 | −12.150 | −58.845 | −62.148 | 1.00 | 69.47 | N |
| ATOM | 2 | CA | GLY | B | 107 | −11.584 | −57.896 | −63.099 | 1.00 | 69.50 | C |
| ATOM | 3 | C | GLY | B | 107 | −11.266 | −56.551 | −62.476 | 1.00 | 69.52 | C |
| ATOM | 4 | O | GLY | B | 107 | −11.271 | −56.407 | −61.253 | 1.00 | 69.51 | O |
| ATOM | 1 | N | THR | B | 108 | −10.989 | −55.562 | −63.322 | 1.00 | 69.51 | N |
| ATOM | 2 | CA | THR | B | 108 | −10.595 | −54.240 | −62.848 | 1.00 | 69.49 | C |
| ATOM | 3 | CB | THR | B | 108 | −9.073 | −54.020 | −62.975 | 1.00 | 69.48 | C |
| ATOM | 4 | OG1 | THR | B | 108 | −8.713 | −53.964 | −64.361 | 1.00 | 69.55 | O |
| ATOM | 5 | CG2 | THR | B | 108 | −8.298 | −55.139 | −62.290 | 1.00 | 69.46 | C |
| ATOM | 6 | C | THR | B | 108 | −11.287 | −53.116 | −63.608 | 1.00 | 69.49 | C |
| ATOM | 7 | O | THR | B | 108 | −12.009 | −53.354 | −64.578 | 1.00 | 69.48 | O |
| ATOM | 1 | N | VAL | B | 109 | −11.046 | −51.890 | −63.150 | 1.00 | 69.49 | N |
| ATOM | 2 | CA | VAL | B | 109 | −11.563 | −50.680 | −63.784 | 1.00 | 69.46 | C |
| ATOM | 3 | CB | VAL | B | 109 | −13.114 | −50.660 | −63.804 | 1.00 | 69.47 | C |
| ATOM | 4 | CG1 | VAL | B | 109 | −13.632 | −49.424 | −64.531 | 1.00 | 69.54 | C |
| ATOM | 5 | CG2 | VAL | B | 109 | −13.680 | −50.750 | −62.389 | 1.00 | 69.55 | C |
| ATOM | 6 | C | VAL | B | 109 | −11.007 | −49.450 | −63.056 | 1.00 | 69.39 | C |
| ATOM | 7 | O | VAL | B | 109 | −11.153 | −49.325 | −61.839 | 1.00 | 69.42 | O |
| ATOM | 1 | N | PRO | B | 110 | −10.343 | −48.548 | −63.799 | 1.00 | 69.28 | N |
| ATOM | 2 | CA | PRO | B | 110 | −9.693 | −47.376 | −63.208 | 1.00 | 69.20 | C |
| ATOM | 3 | CB | PRO | B | 110 | −8.558 | −47.076 | −64.194 | 1.00 | 69.20 | C |
| ATOM | 4 | CG | PRO | B | 110 | −9.001 | −47.667 | −65.506 | 1.00 | 69.19 | C |
| ATOM | 5 | CD | PRO | B | 110 | −10.153 | −48.608 | −65.259 | 1.00 | 69.25 | C |
| ATOM | 6 | C | PRO | B | 110 | −10.598 | −46.150 | −63.049 | 1.00 | 69.09 | C |
| ATOM | 7 | O | PRO | B | 110 | −10.416 | −45.155 | −63.752 | 1.00 | 69.13 | O |
| ATOM | 1 | N | GLY | B | 111 | −11.548 | −46.216 | −62.120 | 1.00 | 68.96 | N |
| ATOM | 2 | CA | GLY | B | 111 | −12.442 | −45.089 | −61.852 | 1.00 | 68.85 | C |
| ATOM | 3 | C | GLY | B | 111 | −13.484 | −44.872 | −62.941 | 1.00 | 68.81 | C |
| ATOM | 4 | O | GLY | B | 111 | −13.344 | −43.965 | −63.800 | 1.00 | 68.65 | O |
| TER | 4 | | GLY | B | 111 | | | | | | |
| ATOM | 1 | N | ASN | B | 130 | −11.159 | −31.270 | −62.225 | 1.00 | 66.96 | N |
| ATOM | 2 | CA | ASN | B | 130 | −9.886 | −30.849 | −61.642 | 1.00 | 67.00 | C |
| ATOM | 3 | CB | ASN | B | 130 | −10.094 | −29.640 | −60.719 | 1.00 | 66.96 | C |
| ATOM | 4 | CG | ASN | B | 130 | −8.779 | −29.020 | −60.264 | 1.00 | 66.90 | C |
| ATOM | 5 | OD1 | ASN | B | 130 | −8.343 | −28.004 | −60.826 | 1.00 | 66.78 | O |
| ATOM | 6 | ND2 | ASN | B | 130 | −8.136 | −29.635 | −59.247 | 1.00 | 66.61 | N |
| ATOM | 7 | C | ASN | B | 130 | −9.150 | −31.973 | −60.895 | 1.00 | 67.05 | C |
| ATOM | 8 | O | ASN | B | 130 | −7.929 | −32.106 | −61.015 | 1.00 | 67.08 | O |
| ATOM | 1 | N | THR | B | 131 | −9.899 | −32.772 | −60.132 | 1.00 | 67.04 | N |
| ATOM | 2 | CA | THR | B | 131 | −9.333 | −33.856 | −59.307 | 1.00 | 66.97 | C |
| ATOM | 3 | CB | THR | B | 131 | −10.400 | −34.450 | −58.342 | 1.00 | 66.99 | C |
| ATOM | 4 | OG1 | THR | B | 131 | −10.043 | −35.792 | −57.984 | 1.00 | 66.77 | O |
| ATOM | 5 | CG2 | THR | B | 131 | −11.777 | −34.460 | −58.998 | 1.00 | 67.17 | C |
| ATOM | 6 | C | THR | B | 131 | −8.678 | −34.980 | −60.126 | 1.00 | 66.94 | C |
| ATOM | 7 | O | THR | B | 131 | −8.847 | −35.054 | −61.348 | 1.00 | 66.93 | O |
| ATOM | 1 | N | PHE | B | 132 | −7.931 | −35.851 | −59.444 | 1.00 | 66.81 | N |
| ATOM | 2 | CA | PHE | B | 132 | −7.174 | −36.912 | −60.115 | 1.00 | 66.68 | C |
| ATOM | 3 | CB | PHE | B | 132 | −5.672 | −36.602 | −60.088 | 1.00 | 66.71 | C |
| ATOM | 4 | CG | PHE | B | 132 | −5.315 | −35.278 | −60.709 | 1.00 | 66.79 | C |
| ATOM | 5 | CD1 | PHE | B | 132 | −4.691 | −34.290 | −59.960 | 1.00 | 66.90 | C |
| ATOM | 6 | CE1 | PHE | B | 132 | −4.363 | −33.064 | −60.532 | 1.00 | 67.27 | C |
| ATOM | 7 | CZ | PHE | B | 132 | −4.666 | −32.814 | −61.865 | 1.00 | 67.02 | C |
| ATOM | 8 | CE2 | PHE | B | 132 | −5.293 | −33.791 | −62.620 | 1.00 | 67.01 | C |
| ATOM | 9 | CD2 | PHE | B | 132 | −5.616 | −35.015 | −62.041 | 1.00 | 66.90 | C |
| ATOM | 10 | C | PHE | B | 132 | −7.447 | −38.316 | −59.563 | 1.00 | 66.58 | C |
| ATOM | 11 | O | PHE | B | 132 | −7.146 | −38.620 | −58.404 | 1.00 | 66.52 | O |
| ATOM | 1 | N | LEU | B | 133 | −8.016 | −39.165 | −60.417 | 1.00 | 66.41 | N |
| ATOM | 2 | CA | LEU | B | 133 | −8.370 | −40.539 | −60.064 | 1.00 | 66.14 | C |
| ATOM | 3 | CB | LEU | B | 133 | −9.874 | −40.769 | −60.256 | 1.00 | 66.19 | C |
| ATOM | 4 | CG | LEU | B | 133 | −10.882 | −40.435 | −59.148 | 1.00 | 66.22 | C |
| ATOM | 5 | CD1 | LEU | B | 133 | −10.589 | −39.104 | −58.467 | 1.00 | 66.32 | C |
| ATOM | 6 | CD2 | LEU | B | 133 | −12.304 | −40.457 | −59.704 | 1.00 | 66.13 | C |
| ATOM | 7 | C | LEU | B | 133 | −7.601 | −41.546 | −60.915 | 1.00 | 65.93 | C |
| ATOM | 8 | O | LEU | B | 133 | −7.829 | −42.751 | −60.820 | 1.00 | 65.82 | O |
| ATOM | 1 | N | ASN | B | 134 | −6.705 | −41.051 | −61.765 | 1.00 | 65.76 | N |
| ATOM | 2 | CA | ASN | B | 134 | −5.909 | −41.930 | −62.615 | 1.00 | 65.55 | C |
| ATOM | 3 | CB | ASN | B | 134 | −5.234 | −41.148 | −63.746 | 1.00 | 65.55 | C |
| ATOM | 4 | CG | ASN | B | 134 | −4.640 | −42.057 | −64.813 | 1.00 | 65.49 | C |
| ATOM | 5 | OD1 | ASN | B | 134 | −3.814 | −41.627 | −65.619 | 1.00 | 65.41 | O |
| ATOM | 6 | ND2 | ASN | B | 134 | −5.060 | −43.319 | −64.821 | 1.00 | 65.26 | N |
| ATOM | 7 | C | ASN | B | 134 | −4.873 | −42.672 | −61.783 | 1.00 | 65.39 | C |
| ATOM | 8 | O | ASN | B | 134 | −4.324 | −43.690 | −62.209 | 1.00 | 65.43 | O |
| ATOM | 1 | N | ARG | B | 135 | −4.612 | −42.146 | −60.590 | 1.00 | 65.09 | N |
| ATOM | 2 | CA | ARG | B | 135 | −3.787 | −42.836 | −59.617 | 1.00 | 64.81 | C |
| ATOM | 3 | CB | ARG | B | 135 | −2.997 | −41.831 | −58.777 | 1.00 | 64.75 | C |
| ATOM | 4 | CG | ARG | B | 135 | −2.260 | −40.714 | −59.006 | 0.00 | 50.00 | C |
| ATOM | 5 | CD | ARG | B | 135 | −1.921 | −39.538 | −58.193 | 0.00 | 50.00 | C |
| ATOM | 6 | NE | ARG | B | 135 | −2.823 | −38.615 | −57.663 | 0.00 | 50.00 | N |
| ATOM | 7 | CZ | ARG | B | 135 | −2.891 | −37.354 | −57.365 | 0.00 | 50.00 | C |
| ATOM | 8 | NH1 | ARG | B | 135 | −3.876 | −36.980 | −56.508 | 0.00 | 50.00 | N |
| ATOM | 9 | NH2 | ARG | B | 135 | −2.196 | −36.373 | −57.896 | 0.00 | 50.00 | N |
| ATOM | 10 | C | ARG | B | 135 | −4.713 | −43.669 | −58.740 | 1.00 | 64.65 | C |
| ATOM | 11 | O | ARG | B | 135 | −4.412 | −43.944 | −57.578 | 1.00 | 64.66 | O |
| ATOM | 1 | N | PHE | B | 136 | −5.844 | −44.072 | −59.314 | 1.00 | 64.44 | N |
| ATOM | 2 | CA | PHE | B | 136 | −6.877 | −44.795 | −58.578 | 1.00 | 64.19 | C |
| ATOM | 3 | CB | PHE | B | 136 | −7.996 | −43.832 | −58.178 | 1.00 | 64.24 | C |
| ATOM | 4 | CG | PHE | B | 136 | −8.564 | −44.089 | −56.815 | 1.00 | 64.14 | C |
| ATOM | 5 | CD1 | PHE | B | 136 | −8.404 | −43.155 | −55.802 | 1.00 | 64.00 | C |
| ATOM | 6 | CE1 | PHE | B | 136 | −8.924 | −43.377 | −54.546 | 1.00 | 64.15 | C |
| ATOM | 7 | CZ | PHE | B | 136 | −9.611 | −44.548 | −54.286 | 1.00 | 64.46 | C |
| ATOM | 8 | CE2 | PHE | B | 136 | −9.777 | −45.491 | −55.290 | 1.00 | 64.42 | C |
| ATOM | 9 | CD2 | PHE | B | 136 | −9.256 | −45.257 | −56.545 | 1.00 | 64.17 | C |
| ATOM | 10 | C | PHE | B | 136 | −7.459 | −45.939 | −59.408 | 1.00 | 63.99 | C |
| ATOM | 11 | O | PHE | B | 136 | −8.050 | −45.718 | −60.469 | 1.00 | 63.97 | O |
| ATOM | 1 | N | MET | B | 137 | −7.293 | −47.160 | −58.911 | 1.00 | 63.68 | N |
| ATOM | 2 | CA | MET | B | 137 | −7.801 | −48.347 | −59.586 | 1.00 | 63.43 | C |
| ATOM | 3 | CB | MET | B | 137 | −6.665 | −49.343 | −59.828 | 1.00 | 63.62 | C |
| ATOM | 4 | CG | MET | B | 137 | −5.684 | −48.929 | −60.916 | 1.00 | 64.35 | C |
| ATOM | 5 | SD | MET | B | 137 | −6.180 | −49.495 | −62.559 | 1.00 | 66.23 | S |
| ATOM | 6 | CE | MET | B | 137 | −6.043 | −51.275 | −62.348 | 1.00 | 65.88 | C |
| ATOM | 7 | C | MET | B | 137 | −8.884 | −49.000 | −58.741 | 1.00 | 63.01 | C |
| ATOM | 8 | O | MET | B | 137 | −8.986 | −48.735 | −57.544 | 1.00 | 63.09 | O |
| ATOM | 1 | N | CYS | B | 138 | −9.688 | −49.857 | −59.362 | 1.00 | 62.44 | N |
| ATOM | 2 | CA | CYS | B | 138 | −10.732 | −50.576 | −58.640 | 1.00 | 61.94 | C |
| ATOM | 3 | CB | CYS | B | 138 | −12.095 | −49.918 | −58.858 | 1.00 | 62.03 | C |
| ATOM | 4 | SG | CYS | B | 138 | −13.490 | −50.936 | −58.322 | 1.00 | 62.33 | S |
| ATOM | 5 | C | CYS | B | 138 | −10.795 | −52.041 | −59.046 | 1.00 | 61.48 | C |
| ATOM | 6 | O | CYS | B | 138 | −10.922 | −52.365 | −60.228 | 1.00 | 61.39 | O |
| ATOM | 1 | N | ALA | B | 139 | −10.711 | −52.921 | −58.055 | 1.00 | 60.93 | N |
| ATOM | 2 | CA | ALA | B | 139 | −10.780 | −54.357 | −58.291 | 1.00 | 60.47 | C |
| ATOM | 3 | CB | ALA | B | 139 | −9.544 | −55.046 | −57.739 | 1.00 | 60.46 | C |
| ATOM | 4 | C | ALA | B | 139 | −12.041 | −54.939 | −57.666 | 1.00 | 60.13 | C |
| ATOM | 5 | O | ALA | B | 139 | −12.487 | −54.485 | −56.612 | 1.00 | 60.12 | O |
| ATOM | 1 | N | GLN | B | 140 | −12.613 | −55.942 | −58.324 | 1.00 | 59.68 | N |
| ATOM | 2 | CA | GLN | B | 140 | −13.850 | −56.562 | −57.868 | 1.00 | 59.22 | C |
| ATOM | 3 | CB | GLN | B | 140 | −15.013 | −56.145 | −58.765 | 1.00 | 59.22 | C |
| ATOM | 4 | CG | GLN | B | 140 | −15.223 | −54.656 | −58.876 | 1.00 | 59.09 | C |
| ATOM | 5 | CD | GLN | B | 140 | −16.449 | −54.321 | −59.685 | 1.00 | 59.09 | C |
| ATOM | 6 | OE1 | GLN | B | 140 | −17.272 | −55.192 | −59.967 | 1.00 | 59.44 | O |
| ATOM | 7 | NE2 | GLN | B | 140 | −16.580 | −53.058 | −60.068 | 1.00 | 59.05 | N |
| ATOM | 8 | C | GLN | B | 140 | −13.731 | −58.074 | −57.908 | 1.00 | 58.94 | C |
| ATOM | 9 | O | GLN | B | 140 | −13.091 | −58.624 | −58.803 | 1.00 | 58.94 | O |
| ATOM | 1 | N | LEU | B | 141 | −14.360 | −58.747 | −56.950 | 1.00 | 58.55 | N |
| ATOM | 2 | CA | LEU | B | 141 | −14.387 | −60.207 | −56.964 | 1.00 | 58.30 | C |
| ATOM | 3 | CB | LEU | B | 141 | −12.981 | −60.785 | −56.744 | 1.00 | 58.30 | C |
| ATOM | 4 | CG | LEU | B | 141 | −12.230 | −60.539 | −55.433 | 1.00 | 58.25 | C |
| ATOM | 5 | CD1 | LEU | B | 141 | −10.894 | −61.267 | −55.464 | 1.00 | 58.11 | C |
| ATOM | 6 | CD2 | LEU | B | 141 | −12.024 | −59.056 | −55.161 | 1.00 | 58.38 | C |
| ATOM | 7 | C | LEU | B | 141 | −15.390 | −60.810 | −55.982 | 1.00 | 58.06 | C |
| ATOM | 8 | O | LEU | B | 141 | −15.453 | −60.403 | −54.825 | 1.00 | 58.07 | O |
| ATOM | 1 | N | PRO | B | 142 | −16.181 | −61.788 | −56.454 | 1.00 | 57.87 | N |
| ATOM | 2 | CA | PRO | B | 142 | −17.163 | −62.505 | −55.644 | 1.00 | 57.70 | C |
| ATOM | 3 | CB | PRO | B | 142 | −17.717 | −63.554 | −56.611 | 1.00 | 57.74 | C |
| ATOM | 4 | CG | PRO | B | 142 | −16.719 | −63.635 | −57.723 | 1.00 | 57.78 | C |
| ATOM | 5 | CD | PRO | B | 142 | −16.164 | −62.265 | −57.846 | 1.00 | 57.80 | C |
| ATOM | 6 | C | PRO | B | 142 | −16.499 | −63.187 | −54.461 | 1.00 | 57.59 | C |
| ATOM | 7 | O | PRO | B | 142 | −15.712 | −64.109 | −54.642 | 1.00 | 57.61 | O |
| ATOM | 1 | N | ASN | B | 143 | −16.827 | −62.729 | −53.258 | 1.00 | 57.52 | N |
| ATOM | 2 | CA | ASN | B | 143 | −16.140 | −63.152 | −52.046 | 1.00 | 57.41 | C |
| ATOM | 3 | CB | ASN | B | 143 | −14.667 | −62.745 | −52.127 | 1.00 | 57.34 | C |
| ATOM | 4 | CG | ASN | B | 143 | −13.903 | −63.022 | −50.846 | 1.00 | 57.28 | C |
| ATOM | 5 | OD1 | ASN | B | 143 | −14.460 | −62.987 | −49.747 | 1.00 | 57.32 | O |
| ATOM | 6 | ND2 | ASN | B | 143 | −12.609 | −63.281 | −50.982 | 1.00 | 56.70 | N |
| ATOM | 7 | C | ASN | B | 143 | −16.814 | −62.516 | −50.833 | 1.00 | 57.47 | C |
| ATOM | 8 | O | ASN | B | 143 | −16.943 | −61.295 | −50.759 | 1.00 | 57.41 | O |
| ATOM | 1 | N | GLN | B | 144 | −17.237 | −63.350 | −49.885 | 1.00 | 57.56 | N |
| ATOM | 2 | CA | GLN | B | 144 | −18.063 | −62.908 | −48.754 | 1.00 | 57.72 | C |
| ATOM | 3 | CB | GLN | B | 144 | −18.273 | −64.055 | −47.773 | 1.00 | 57.81 | C |
| ATOM | 4 | CG | GLN | B | 144 | −18.601 | −65.378 | −48.416 | 1.00 | 58.02 | C |
| ATOM | 5 | CD | GLN | B | 144 | −18.968 | −66.419 | −47.386 | 1.00 | 58.57 | C |
| ATOM | 6 | OE1 | GLN | B | 144 | −19.865 | −66.207 | −46.566 | 1.00 | 58.64 | O |
| ATOM | 7 | NE2 | GLN | B | 144 | −18.274 | −67.553 | −47.417 | 1.00 | 58.83 | N |
| ATOM | 8 | C | GLN | B | 144 | −17.496 | −61.713 | −47.992 | 1.00 | 57.73 | C |
| ATOM | 9 | O | GLN | B | 144 | −18.229 | −60.782 | −47.618 | 1.00 | 57.88 | O |
| ATOM | 1 | N | VAL | B | 145 | −16.193 | −61.756 | −47.740 | 1.00 | 57.64 | N |
| ATOM | 2 | CA | VAL | B | 145 | −15.537 | −60.663 | −47.053 | 1.00 | 57.55 | C |
| ATOM | 3 | CB | VAL | B | 145 | −14.015 | −60.834 | −47.034 | 1.00 | 57.60 | C |
| ATOM | 4 | CG1 | VAL | B | 145 | −13.649 | −62.269 | −46.694 | 1.00 | 57.62 | C |
| ATOM | 5 | CG2 | VAL | B | 145 | −13.391 | −59.867 | −46.039 | 1.00 | 57.54 | C |
| ATOM | 6 | C | VAL | B | 145 | −15.896 | −59.369 | −47.760 | 1.00 | 57.47 | C |
| ATOM | 7 | O | VAL | B | 145 | −16.252 | −58.386 | −47.119 | 1.00 | 57.53 | O |
| ATOM | 1 | N | LEU | B | 146 | −15.825 | −59.386 | −49.086 | 1.00 | 57.46 | N |
| ATOM | 2 | CA | LEU | B | 146 | −16.195 | −58.222 | −49.882 | 1.00 | 57.59 | C |
| ATOM | 3 | CB | LEU | B | 146 | −15.718 | −58.367 | −51.330 | 1.00 | 57.59 | C |
| ATOM | 4 | CG | LEU | B | 146 | −14.250 | −58.045 | −51.620 | 1.00 | 57.56 | C |
| ATOM | 5 | CD1 | LEU | B | 146 | −13.770 | −56.879 | −50.764 | 1.00 | 57.23 | C |
| ATOM | 6 | CD2 | LEU | B | 146 | −13.382 | −59.266 | −51.394 | 1.00 | 57.83 | C |
| ATOM | 7 | C | LEU | B | 146 | −17.697 | −57.934 | −49.837 | 1.00 | 57.65 | C |
| ATOM | 8 | O | LEU | B | 146 | −18.135 | −56.840 | −50.185 | 1.00 | 57.62 | O |
| ATOM | 1 | N | GLU | B | 147 | −18.485 | −58.917 | −49.417 | 1.00 | 57.74 | N |
| ATOM | 2 | CA | GLU | B | 147 | −19.896 | −58.681 | −49.164 | 1.00 | 57.96 | C |
| ATOM | 3 | CB | GLU | B | 147 | −20.652 | −59.994 | −48.955 | 1.00 | 57.87 | C |
| ATOM | 4 | CG | GLU | B | 147 | −20.690 | −60.912 | −50.158 | 1.00 | 58.25 | C |
| ATOM | 5 | CD | GLU | B | 147 | −21.327 | −62.253 | −49.835 | 1.00 | 58.44 | C |
| ATOM | 6 | OE1 | GLU | B | 147 | −21.131 | −63.217 | −50.612 | 1.00 | 58.79 | O |
| ATOM | 7 | OE2 | GLU | B | 147 | −22.020 | −62.343 | −48.797 | 1.00 | 59.30 | O |
| ATOM | 8 | C | GLU | B | 147 | −20.020 | −57.827 | −47.914 | 1.00 | 57.91 | C |
| ATOM | 9 | O | GLU | B | 147 | −20.766 | −56.850 | −47.887 | 1.00 | 58.03 | O |
| ATOM | 1 | N | SER | B | 148 | −19.281 | −58.197 | −46.874 | 1.00 | 57.86 | N |
| ATOM | 2 | CA | SER | B | 148 | −19.375 | −57.474 | −45.609 | 1.00 | 57.89 | C |
| ATOM | 3 | CB | SER | B | 148 | −18.770 | −58.296 | −44.474 | 1.00 | 57.92 | C |
| ATOM | 4 | OG | SER | B | 148 | −19.358 | −59.582 | −44.412 | 1.00 | 58.15 | O |
| ATOM | 5 | C | SER | B | 148 | −18.720 | −56.091 | −45.661 | 1.00 | 57.91 | C |
| ATOM | 6 | O | SER | B | 148 | −19.316 | −55.099 | −45.242 | 1.00 | 58.01 | O |
| ATOM | 1 | N | ILE | B | 149 | −17.495 | −56.031 | −46.175 | 1.00 | 57.82 | N |
| ATOM | 2 | CA | ILE | B | 149 | −16.716 | −54.795 | −46.184 | 1.00 | 57.70 | C |
| ATOM | 3 | CB | ILE | B | 149 | −15.661 | −54.802 | −45.065 | 1.00 | 57.63 | C |
| ATOM | 4 | CG1 | ILE | B | 149 | −14.680 | −55.962 | −45.263 | 1.00 | 57.21 | C |
| ATOM | 5 | CD1 | ILE | B | 149 | −13.512 | −55.946 | −44.303 | 1.00 | 56.31 | C |
| ATOM | 6 | CG2 | ILE | B | 149 | −16.325 | −54.901 | −43.708 | 1.00 | 57.78 | C |
| ATOM | 7 | C | ILE | B | 149 | −15.984 | −54.602 | −47.505 | 1.00 | 57.81 | C |
| ATOM | 8 | O | ILE | B | 149 | −16.105 | −55.418 | −48.416 | 1.00 | 58.00 | O |
| ATOM | 1 | N | SER | B | 150 | −15.225 | −53.517 | −47.611 | 1.00 | 57.80 | N |
| ATOM | 2 | CA | SER | B | 150 | −14.308 | −53.345 | −48.737 | 1.00 | 57.87 | C |
| ATOM | 3 | CB | SER | B | 150 | −14.924 | −52.492 | −49.852 | 1.00 | 57.80 | C |
| ATOM | 4 | OG | SER | B | 150 | −15.622 | −51.381 | −49.328 | 1.00 | 58.07 | O |
| ATOM | 5 | C | SER | B | 150 | −12.972 | −52.780 | −48.263 | 1.00 | 57.82 | C |
| ATOM | 6 | O | SER | B | 150 | −12.917 | −51.969 | −47.337 | 1.00 | 57.84 | O |
| ATOM | 1 | N | ILE | B | 151 | −11.893 | −53.233 | −48.889 | 1.00 | 57.75 | N |
| ATOM | 2 | CA | ILE | B | 151 | −10.556 | −52.907 | −48.422 | 1.00 | 57.75 | C |
| ATOM | 3 | CB | ILE | B | 151 | −9.748 | −54.178 | −48.150 | 1.00 | 57.69 | C |
| ATOM | 4 | CG1 | ILE | B | 151 | −10.576 | −55.163 | −47.328 | 1.00 | 57.79 | C |
| ATOM | 5 | CD1 | ILE | B | 151 | −10.080 | −56.585 | −47.412 | 1.00 | 58.36 | C |
| ATOM | 6 | CG2 | ILE | B | 151 | −8.446 | −53.837 | −47.446 | 1.00 | 57.61 | C |
| ATOM | 7 | C | ILE | B | 151 | −9.807 | −52.074 | −49.445 | 1.00 | 57.80 | C |
| ATOM | 8 | O | ILE | B | 151 | −9.608 | −52.502 | −50.581 | 1.00 | 57.77 | O |
| ATOM | 1 | N | ILE | B | 152 | −9.384 | −50.882 | −49.045 | 1.00 | 57.79 | N |
| ATOM | 2 | CA | ILE | B | 152 | −8.629 | −50.046 | −49.955 | 1.00 | 57.80 | C |
| ATOM | 3 | CB | ILE | B | 152 | −9.093 | −48.573 | −49.903 | 1.00 | 57.84 | C |
| ATOM | 4 | CG1 | ILE | B | 152 | −8.002 | −47.669 | −49.334 | 1.00 | 58.21 | C |
| ATOM | 5 | CD1 | ILE | B | 152 | −6.996 | −47.213 | −50.382 | 1.00 | 58.80 | C |
| ATOM | 6 | CG2 | ILE | B | 152 | −10.418 | −48.445 | −49.155 | 1.00 | 57.83 | C |
| ATOM | 7 | C | ILE | B | 152 | −7.133 | −50.189 | −49.669 | 1.00 | 57.75 | C |
| ATOM | 8 | O | ILE | B | 152 | −6.677 | −49.991 | −48.543 | 1.00 | 57.62 | O |
| ATOM | 1 | N | ASP | B | 153 | −6.386 | −50.564 | −50.702 | 1.00 | 57.80 | N |
| ATOM | 2 | CA | ASP | B | 153 | −4.950 | −50.776 | −50.600 | 1.00 | 57.91 | C |
| ATOM | 3 | CB | ASP | B | 153 | −4.548 | −52.005 | −51.413 | 1.00 | 57.99 | C |
| ATOM | 4 | CG | ASP | B | 153 | −3.082 | −52.358 | −51.258 | 1.00 | 58.23 | C |
| ATOM | 5 | OD1 | ASP | B | 153 | −2.331 | −51.554 | −50.664 | 1.00 | 58.41 | O |
| ATOM | 6 | OD2 | ASP | B | 153 | −2.681 | −53.444 | −51.736 | 1.00 | 58.26 | O |
| ATOM | 7 | C | ASP | B | 153 | −4.210 | −49.549 | −51.113 | 1.00 | 58.00 | C |
| ATOM | 8 | O | ASP | B | 153 | −4.584 | −48.975 | −52.137 | 1.00 | 58.18 | O |
| ATOM | 1 | N | THR | B | 154 | −3.152 | −49.160 | −50.408 | 1.00 | 58.02 | N |
| ATOM | 2 | CA | THR | B | 154 | −2.458 | −47.904 | −50.683 | 1.00 | 57.95 | C |
| ATOM | 3 | CB | THR | B | 154 | −2.314 | −47.058 | −49.401 | 1.00 | 57.97 | C |
| ATOM | 4 | OG1 | THR | B | 154 | −1.536 | −47.776 | −48.429 | 1.00 | 57.67 | O |
| ATOM | 5 | CG2 | THR | B | 154 | −3.682 | −46.726 | −48.824 | 1.00 | 57.86 | C |
| ATOM | 6 | C | THR | B | 154 | −1.068 | −48.117 | −51.266 | 1.00 | 58.01 | C |
| ATOM | 7 | O | THR | B | 154 | −0.508 | −49.205 | −51.144 | 1.00 | 57.95 | O |
| ATOM | 1 | N | PRO | B | 155 | −0.509 | −47.071 | −51.906 | 1.00 | 58.12 | N |
| ATOM | 2 | CA | PRO | B | 155 | 0.886 | −47.083 | −52.338 | 1.00 | 58.12 | C |
| ATOM | 3 | CB | PRO | B | 155 | 1.042 | −45.757 | −53.095 | 1.00 | 58.01 | C |
| ATOM | 4 | CG | PRO | B | 155 | −0.338 | −45.284 | −53.372 | 1.00 | 58.04 | C |
| ATOM | 5 | CD | PRO | B | 155 | −1.184 | −45.812 | −52.267 | 1.00 | 58.18 | C |
| ATOM | 6 | C | PRO | B | 155 | 1.818 | −47.093 | −51.136 | 1.00 | 58.22 | C |
| ATOM | 7 | O | PRO | B | 155 | 1.450 | −46.623 | −50.054 | 1.00 | 58.19 | O |
| ATOM | 1 | N | GLY | B | 156 | 3.017 | −47.628 | −51.331 | 1.00 | 58.35 | N |
| ATOM | 2 | CA | GLY | B | 156 | 4.008 | −47.689 | −50.267 | 1.00 | 58.61 | C |
| ATOM | 3 | C | GLY | B | 156 | 4.562 | −46.324 | −49.920 | 1.00 | 58.79 | C |
| ATOM | 4 | O | GLY | B | 156 | 4.955 | −45.557 | −50.800 | 1.00 | 58.87 | O |
| ATOM | 1 | N | ILE | B | 157 | 4.584 | −46.016 | −48.630 | 1.00 | 58.96 | N |
| ATOM | 2 | CA | ILE | B | 157 | 5.160 | −44.769 | −48.170 | 1.00 | 59.18 | C |
| ATOM | 3 | CB | ILE | B | 157 | 4.868 | −44.544 | −46.684 | 1.00 | 59.11 | C |
| ATOM | 4 | CG1 | ILE | B | 157 | 3.363 | −44.645 | −46.436 | 1.00 | 59.27 | C |
| ATOM | 5 | CD1 | ILE | B | 157 | 2.949 | −44.359 | −45.008 | 1.00 | 59.64 | C |
| ATOM | 6 | CG2 | ILE | B | 157 | 5.373 | −43.190 | −46.243 | 1.00 | 59.13 | C |
| ATOM | 7 | C | ILE | B | 157 | 6.662 | −44.792 | −48.424 | 1.00 | 59.40 | C |
| ATOM | 8 | O | ILE | B | 157 | 7.355 | −45.715 | −48.006 | 1.00 | 59.29 | O |
| ATOM | 1 | N | LEU | B | 158 | 7.156 | −43.786 | −49.138 | 1.00 | 59.83 | N |
| ATOM | 2 | CA | LEU | B | 158 | 8.576 | −43.723 | −49.475 | 1.00 | 60.23 | C |
| ATOM | 3 | CB | LEU | B | 158 | 8.886 | −42.471 | −50.303 | 1.00 | 60.13 | C |
| ATOM | 4 | CG | LEU | B | 158 | 9.487 | −41.410 | −49.914 | 0.00 | 50.00 | C |
| ATOM | 5 | CD1 | LEU | B | 158 | 10.336 | −41.219 | −48.711 | 0.00 | 50.00 | C |
| ATOM | 6 | CD2 | LEU | B | 158 | 10.108 | −40.676 | −51.109 | 0.00 | 50.00 | C |
| ATOM | 7 | C | LEU | B | 158 | 9.437 | −43.761 | −48.215 | 1.00 | 60.49 | C |
| ATOM | 8 | O | LEU | B | 158 | 9.123 | −43.110 | −47.217 | 1.00 | 60.52 | O |
| ATOM | 1 | N | SER | B | 159 | 10.516 | −44.535 | −48.265 | 1.00 | 60.79 | N |
| ATOM | 2 | CA | SER | B | 159 | 11.430 | −44.651 | −47.135 | 1.00 | 61.07 | C |
| ATOM | 3 | CB | SER | B | 159 | 12.365 | −45.849 | −47.322 | 1.00 | 61.20 | C |
| ATOM | 4 | OG | SER | B | 159 | 13.023 | −46.173 | −46.109 | 1.00 | 61.72 | O |
| ATOM | 5 | C | SER | B | 159 | 12.240 | −43.364 | −46.941 | 1.00 | 61.06 | C |
| ATOM | 6 | O | SER | B | 159 | 13.457 | −43.338 | −47.150 | 1.00 | 61.02 | O |
| TER | 6 | | SER | B | 159 | | | | | | |
| ATOM | 1 | N | ARG | B | 167 | 6.924 | −37.115 | −55.151 | 1.00 | 65.73 | N |
| ATOM | 2 | CA | ARG | B | 167 | 6.392 | −35.811 | −55.537 | 1.00 | 65.83 | C |
| ATOM | 3 | CB | ARG | B | 167 | 7.500 | −34.902 | −56.103 | 1.00 | 65.92 | C |
| ATOM | 4 | CG | ARG | B | 167 | 8.650 | −34.591 | −55.146 | 1.00 | 66.25 | C |
| ATOM | 5 | CD | ARG | B | 167 | 9.883 | −35.448 | −55.436 | 1.00 | 66.72 | C |
| ATOM | 6 | NE | ARG | B | 167 | 9.774 | −36.803 | −54.884 | 1.00 | 67.11 | N |
| ATOM | 7 | CZ | ARG | B | 167 | 10.682 | −37.760 | −55.064 | 1.00 | 67.11 | C |
| ATOM | 8 | NH1 | ARG | B | 167 | 11.772 | −37.514 | −55.788 | 1.00 | 67.24 | N |
| ATOM | 9 | NH2 | ARG | B | 167 | 10.502 | −38.961 | −54.523 | 1.00 | 66.65 | N |
| ATOM | 10 | C | ARG | B | 167 | 5.283 | −35.961 | −56.573 | 1.00 | 65.75 | C |
| ATOM | 11 | O | ARG | B | 167 | 5.467 | −35.617 | −57.741 | 1.00 | 65.80 | O |
| ATOM | 1 | N | GLY | B | 168 | 4.133 | −36.474 | −56.149 | 1.00 | 65.67 | N |
| ATOM | 2 | CA | GLY | B | 168 | 3.002 | −36.639 | −57.055 | 1.00 | 65.55 | C |
| ATOM | 3 | C | GLY | B | 168 | 1.748 | −35.936 | −56.574 | 1.00 | 65.49 | C |
| ATOM | 4 | O | GLY | B | 168 | 1.157 | −35.132 | −57.293 | 1.00 | 65.38 | O |
| ATOM | 1 | N | TYR | B | 169 | 1.349 | −36.239 | −55.345 | 1.00 | 65.52 | N |
| ATOM | 2 | CA | TYR | B | 169 | 0.108 | −35.720 | −54.781 | 1.00 | 65.52 | C |
| ATOM | 3 | CB | TYR | B | 169 | −1.020 | −36.744 | −54.967 | 1.00 | 65.60 | C |
| ATOM | 4 | CG | TYR | B | 169 | −0.560 | −38.185 | −54.862 | 1.00 | 65.63 | C |
| ATOM | 5 | CD1 | TYR | B | 169 | −0.309 | −38.940 | −56.004 | 1.00 | 65.74 | C |
| ATOM | 6 | CE1 | TYR | B | 169 | 0.118 | −40.256 | −55.915 | 1.00 | 65.83 | C |
| ATOM | 7 | CZ | TYR | B | 169 | 0.302 | −40.834 | −54.673 | 1.00 | 65.80 | C |
| ATOM | 8 | OH | TYR | B | 169 | 0.728 | −42.140 | −54.582 | 1.00 | 65.64 | O |
| ATOM | 9 | CE2 | TYR | B | 169 | 0.062 | −40.105 | −53.523 | 1.00 | 65.77 | C |
| ATOM | 10 | CD2 | TYR | B | 169 | −0.366 | −38.788 | −53.623 | 1.00 | 65.73 | C |
| ATOM | 11 | C | TYR | B | 169 | 0.284 | −35.383 | −53.298 | 1.00 | 65.44 | C |
| ATOM | 12 | O | TYR | B | 169 | 1.393 | −35.459 | −52.765 | 1.00 | 65.44 | O |
| ATOM | 1 | N | ASP | B | 170 | −0.812 | −35.007 | −52.641 | 1.00 | 65.30 | N |
| ATOM | 2 | CA | ASP | B | 170 | −0.794 | −34.711 | −51.209 | 1.00 | 65.09 | C |
| ATOM | 3 | CB | ASP | B | 170 | −1.747 | −33.557 | −50.884 | 1.00 | 65.12 | C |
| ATOM | 4 | CG | ASP | B | 170 | −1.398 | −32.862 | −49.579 | 1.00 | 65.25 | C |
| ATOM | 5 | OD1 | ASP | B | 170 | −1.812 | −31.695 | −49.399 | 1.00 | 65.06 | O |
| ATOM | 6 | OD2 | ASP | B | 170 | −0.711 | −33.481 | −48.734 | 1.00 | 65.55 | O |
| ATOM | 7 | C | ASP | B | 170 | −1.137 | −35.965 | −50.397 | 1.00 | 64.95 | C |
| ATOM | 8 | O | ASP | B | 170 | −2.198 | −36.058 | −49.752 | 1.00 | 64.78 | O |
| ATOM | 1 | N | PHE | B | 171 | −0.219 | −36.926 | −50.441 | 1.00 | 64.77 | N |
| ATOM | 2 | CA | PHE | B | 171 | −0.407 | −38.217 | −49.795 | 1.00 | 64.63 | C |
| ATOM | 3 | CB | PHE | B | 171 | 0.920 | −38.975 | −49.718 | 1.00 | 64.63 | C |
| ATOM | 4 | CG | PHE | B | 171 | 0.772 | −40.414 | −49.323 | 1.00 | 64.58 | C |
| ATOM | 5 | CD1 | PHE | B | 171 | 0.020 | −41.283 | −50.095 | 1.00 | 64.37 | C |
| ATOM | 6 | CE1 | PHE | B | 171 | −0.116 | −42.608 | −49.737 | 1.00 | 64.43 | C |
| ATOM | 7 | CZ | PHE | B | 171 | 0.508 | −43.083 | −48.598 | 1.00 | 64.73 | C |
| ATOM | 8 | CE2 | PHE | B | 171 | 1.263 | −42.228 | −47.821 | 1.00 | 64.67 | C |
| ATOM | 9 | CD2 | PHE | B | 171 | 1.393 | −40.902 | −48.186 | 1.00 | 64.81 | C |
| ATOM | 10 | C | PHE | B | 171 | −1.034 | −38.064 | −48.413 | 1.00 | 64.56 | C |
| ATOM | 11 | O | PHE | B | 171 | −2.109 | −38.616 | −48.149 | 1.00 | 64.43 | O |
| ATOM | 1 | N | PRO | B | 172 | −0.372 | −37.306 | −47.526 | 1.00 | 64.52 | N |
| ATOM | 2 | CA | PRO | B | 172 | −0.955 | −37.032 | −46.220 | 1.00 | 64.49 | C |
| ATOM | 3 | CB | PRO | B | 172 | −0.073 | −35.907 | −45.681 | 1.00 | 64.54 | C |
| ATOM | 4 | CG | PRO | B | 172 | 1.254 | −36.174 | −46.298 | 1.00 | 64.64 | C |
| ATOM | 5 | CD | PRO | B | 172 | 0.953 | −36.678 | −47.680 | 1.00 | 64.57 | C |
| ATOM | 6 | C | PRO | B | 172 | −2.416 | −36.583 | −46.312 | 1.00 | 64.39 | C |
| ATOM | 7 | O | PRO | B | 172 | −3.275 | −37.148 | −45.625 | 1.00 | 64.27 | O |
| ATOM | 1 | N | ALA | B | 173 | −2.700 | −35.592 | −47.154 | 1.00 | 64.18 | N |
| ATOM | 2 | CA | ALA | B | 173 | −4.068 | −35.093 | −47.287 | 1.00 | 64.15 | C |
| ATOM | 3 | CB | ALA | B | 173 | −4.141 | −33.959 | −48.291 | 1.00 | 64.18 | C |
| ATOM | 4 | C | ALA | B | 173 | −5.047 | −36.206 | −47.657 | 1.00 | 64.15 | C |
| ATOM | 5 | O | ALA | B | 173 | −6.136 | −36.305 | −47.074 | 1.00 | 64.04 | O |
| ATOM | 1 | N | VAL | B | 174 | −4.668 | −37.051 | −48.613 | 1.00 | 64.23 | N |
| ATOM | 2 | CA | VAL | B | 174 | −5.545 | −38.177 | −48.961 | 1.00 | 64.12 | C |
| ATOM | 3 | CB | VAL | B | 174 | −5.127 | −38.899 | −50.275 | 1.00 | 64.14 | C |
| ATOM | 4 | CG1 | VAL | B | 174 | −3.689 | −38.598 | −50.637 | 1.00 | 64.11 | C |
| ATOM | 5 | CG2 | VAL | B | 174 | −5.374 | −40.397 | −50.178 | 1.00 | 64.15 | C |
| ATOM | 6 | C | VAL | B | 174 | −5.719 | −39.162 | −47.791 | 1.00 | 64.08 | C |
| ATOM | 7 | O | VAL | B | 174 | −6.840 | −39.617 | −47.505 | 1.00 | 63.98 | O |
| ATOM | 1 | N | LEU | B | 175 | −4.621 | −39.466 | −47.102 | 1.00 | 64.02 | N |
| ATOM | 2 | CA | LEU | B | 175 | −4.700 | −40.298 | −45.907 | 1.00 | 63.80 | C |
| ATOM | 3 | CB | LEU | B | 175 | −3.313 | −40.556 | −45.326 | 1.00 | 63.79 | C |
| ATOM | 4 | CG | LEU | B | 175 | −2.668 | −41.866 | −45.780 | 1.00 | 64.02 | C |
| ATOM | 5 | CD1 | LEU | B | 175 | −1.260 | −42.013 | −45.220 | 1.00 | 64.02 | C |
| ATOM | 6 | CD2 | LEU | B | 175 | −3.534 | −43.050 | −45.371 | 1.00 | 64.22 | C |
| ATOM | 7 | C | LEU | B | 175 | −5.607 | −39.679 | −44.849 | 1.00 | 63.78 | C |
| ATOM | 8 | O | LEU | B | 175 | −6.233 | −40.386 | −44.063 | 1.00 | 63.66 | O |
| ATOM | 1 | N | ARG | B | 176 | −5.681 | −38.355 | −44.834 | 1.00 | 63.87 | N |
| ATOM | 2 | CA | ARG | B | 176 | −6.550 | −37.663 | −43.894 | 1.00 | 63.82 | C |
| ATOM | 3 | CB | ARG | B | 176 | −6.079 | −36.223 | −43.697 | 1.00 | 63.86 | C |
| ATOM | 4 | CG | ARG | B | 176 | −6.952 | −35.376 | −42.788 | 1.00 | 64.46 | C |
| ATOM | 5 | CD | ARG | B | 176 | −6.216 | −34.101 | −42.406 | 1.00 | 65.87 | C |
| ATOM | 6 | NE | ARG | B | 176 | −5.005 | −33.929 | −43.214 | 1.00 | 66.91 | N |
| ATOM | 7 | CZ | ARG | B | 176 | −4.418 | −32.760 | −43.460 | 1.00 | 67.30 | C |
| ATOM | 8 | NH1 | ARG | B | 176 | −3.317 | −32.717 | −44.204 | 1.00 | 67.30 | N |
| ATOM | 9 | NH2 | ARG | B | 176 | −4.935 | −31.635 | −42.971 | 1.00 | 67.62 | N |
| ATOM | 10 | C | ARG | B | 176 | −8.010 | −37.722 | −44.345 | 1.00 | 63.65 | C |
| ATOM | 11 | O | ARG | B | 176 | −8.917 | −37.859 | −43.520 | 1.00 | 63.59 | O |
| ATOM | 1 | N | TRP | B | 177 | −8.233 | −37.633 | −45.654 | 1.00 | 63.54 | N |
| ATOM | 2 | CA | TRP | B | 177 | −9.580 | −37.773 | −46.215 | 1.00 | 63.37 | C |
| ATOM | 3 | CB | TRP | B | 177 | −9.525 | −37.527 | −47.723 | 1.00 | 63.31 | C |
| ATOM | 4 | CG | TRP | B | 177 | −10.847 | −37.583 | −48.429 | 1.00 | 63.26 | C |
| ATOM | 5 | CD1 | TRP | B | 177 | −11.718 | −36.549 | −48.628 | 1.00 | 62.97 | C |
| ATOM | 6 | NE1 | TRP | B | 177 | −12.817 | −36.985 | −49.328 | 1.00 | 62.77 | N |
| ATOM | 7 | CE2 | TRP | B | 177 | −12.669 | −38.320 | −49.603 | 1.00 | 63.09 | C |
| ATOM | 8 | CD2 | TRP | B | 177 | −11.437 | −38.731 | −49.055 | 1.00 | 63.35 | C |
| ATOM | 9 | CE3 | TRP | B | 177 | −11.044 | −40.069 | −49.200 | 1.00 | 63.27 | C |
| ATOM | 10 | CZ3 | TRP | B | 177 | −11.883 | −40.939 | −49.878 | 1.00 | 63.03 | C |
| ATOM | 11 | CH2 | TRP | B | 177 | −13.104 | −40.500 | −50.410 | 1.00 | 63.04 | C |
| ATOM | 12 | CZ2 | TRP | B | 177 | −13.514 | −39.199 | −50.284 | 1.00 | 63.18 | C |
| ATOM | 13 | C | TRP | B | 177 | −10.141 | −39.168 | −45.912 | 1.00 | 63.41 | C |
| ATOM | 14 | O | TRP | B | 177 | −11.280 | −39.331 | −45.407 | 1.00 | 63.24 | O |
| ATOM | 1 | N | PHE | B | 178 | −9.326 | −40.177 | −46.211 | 1.00 | 63.59 | N |
| ATOM | 2 | CA | PHE | B | 178 | −9.661 | −41.545 | −45.836 | 1.00 | 63.57 | C |
| ATOM | 3 | CB | PHE | B | 178 | −8.594 | −42.526 | −46.325 | 1.00 | 63.59 | C |
| ATOM | 4 | CG | PHE | B | 178 | −8.785 | −42.971 | −47.746 | 1.00 | 63.89 | C |
| ATOM | 5 | CD1 | PHE | B | 178 | −9.901 | −43.713 | −48.111 | 1.00 | 64.14 | C |
| ATOM | 6 | CE1 | PHE | B | 178 | −10.080 | −44.131 | −49.422 | 1.00 | 64.00 | C |
| ATOM | 7 | CZ | PHE | B | 178 | −9.138 | −43.811 | −50.382 | 1.00 | 63.93 | C |
| ATOM | 8 | CE2 | PHE | B | 178 | −8.020 | −43.076 | −50.032 | 1.00 | 64.17 | C |
| ATOM | 9 | CD2 | PHE | B | 178 | −7.846 | −42.660 | −48.717 | 1.00 | 64.18 | C |
| ATOM | 10 | C | PHE | B | 178 | −9.845 | −41.661 | −44.321 | 1.00 | 63.55 | C |
| ATOM | 11 | O | PHE | B | 178 | −10.841 | −42.210 | −43.851 | 1.00 | 63.61 | O |
| ATOM | 1 | N | ALA | B | 179 | −8.891 | −41.133 | −43.558 | 1.00 | 63.51 | N |
| ATOM | 2 | CA | ALA | B | 179 | −8.979 | −41.170 | −42.100 | 1.00 | 63.38 | C |
| ATOM | 3 | CB | ALA | B | 179 | −7.844 | −40.377 | −41.473 | 1.00 | 63.38 | C |
| ATOM | 4 | C | ALA | B | 179 | −10.332 | −40.648 | −41.623 | 1.00 | 63.30 | C |
| ATOM | 5 | O | ALA | B | 179 | −10.935 | −41.207 | −40.705 | 1.00 | 63.20 | O |
| ATOM | 1 | N | GLU | B | 180 | −10.803 | −39.574 | −42.252 | 1.00 | 63.33 | N |
| ATOM | 2 | CA | GLU | B | 180 | −12.135 | −39.051 | −41.967 | 1.00 | 63.25 | C |
| ATOM | 3 | CB | GLU | B | 180 | −12.423 | −37.789 | −42.781 | 1.00 | 63.23 | C |
| ATOM | 4 | CG | GLU | B | 180 | −11.732 | −36.535 | −42.289 | 1.00 | 63.57 | C |
| ATOM | 5 | CD | GLU | B | 180 | −11.933 | −35.365 | −43.233 | 1.00 | 63.57 | C |
| ATOM | 6 | OE1 | GLU | B | 180 | −12.583 | −35.556 | −44.287 | 1.00 | 63.87 | O |
| ATOM | 7 | OE2 | GLU | B | 180 | −11.436 | −34.258 | −42.924 | 1.00 | 64.08 | O |
| ATOM | 8 | C | GLU | B | 180 | −13.195 | −40.090 | −42.288 | 1.00 | 63.07 | C |
| ATOM | 9 | O | GLU | B | 180 | −14.088 | −40.338 | −41.478 | 1.00 | 63.03 | O |
| ATOM | 1 | N | ARG | B | 181 | −13.101 | −40.696 | −43.472 | 1.00 | 62.95 | N |
| ATOM | 2 | CA | ARG | B | 181 | −14.172 | −41.605 | −43.916 | 1.00 | 62.57 | C |
| ATOM | 3 | CB | ARG | B | 181 | −14.320 | −41.556 | −45.436 | 1.00 | 62.55 | C |
| ATOM | 4 | CG | ARG | B | 181 | −15.181 | −40.405 | −45.903 | 1.00 | 62.92 | C |
| ATOM | 5 | CD | ARG | B | 181 | −14.691 | −39.845 | −47.215 | 1.00 | 63.49 | C |
| ATOM | 6 | NE | ARG | B | 181 | −15.103 | −38.454 | −47.389 | 1.00 | 64.41 | N |
| ATOM | 7 | CZ | ARG | B | 181 | −14.496 | −37.415 | −46.820 | 1.00 | 64.92 | C |
| ATOM | 8 | NH1 | ARG | B | 181 | −14.938 | −36.180 | −47.037 | 1.00 | 65.08 | N |
| ATOM | 9 | NH2 | ARG | B | 181 | −13.443 | −37.606 | −46.034 | 1.00 | 65.13 | N |
| ATOM | 10 | C | ARG | B | 181 | −14.087 | −43.059 | −43.439 | 1.00 | 62.33 | C |
| ATOM | 11 | O | ARG | B | 181 | −15.104 | −43.668 | −43.115 | 1.00 | 62.41 | O |
| ATOM | 1 | N | VAL | B | 182 | −12.877 | −43.601 | −43.384 | 1.00 | 62.04 | N |
| ATOM | 2 | CA | VAL | B | 182 | −12.653 | −45.040 | −43.220 | 1.00 | 61.56 | C |
| ATOM | 3 | CB | VAL | B | 182 | −11.218 | −45.378 | −43.634 | 1.00 | 61.45 | C |
| ATOM | 4 | CG1 | VAL | B | 182 | −10.698 | −46.552 | −42.853 | 1.00 | 61.55 | C |
| ATOM | 5 | CG2 | VAL | B | 182 | −11.154 | −45.623 | −45.131 | 1.00 | 61.14 | C |
| ATOM | 6 | C | VAL | B | 182 | −12.930 | −45.596 | −41.818 | 1.00 | 61.44 | C |
| ATOM | 7 | O | VAL | B | 182 | −12.831 | −44.875 | −40.828 | 1.00 | 61.47 | O |
| ATOM | 1 | N | ASP | B | 183 | −13.272 | −46.882 | −41.746 | 1.00 | 61.33 | N |
| ATOM | 2 | CA | ASP | B | 183 | −13.576 | −47.554 | −40.468 | 1.00 | 61.35 | C |
| ATOM | 3 | CB | ASP | B | 183 | −14.500 | −48.762 | −40.685 | 1.00 | 61.39 | C |
| ATOM | 4 | CG | ASP | B | 183 | −15.966 | −48.373 | −40.747 | 1.00 | 62.09 | C |
| ATOM | 5 | OD1 | ASP | B | 183 | −16.748 | −48.863 | −39.902 | 1.00 | 62.54 | O |
| ATOM | 6 | OD2 | ASP | B | 183 | −16.338 | −47.569 | −41.631 | 1.00 | 63.12 | O |
| ATOM | 7 | C | ASP | B | 183 | −12.342 | −47.992 | −39.667 | 1.00 | 61.13 | C |
| ATOM | 8 | O | ASP | B | 183 | −12.271 | −47.778 | −38.455 | 1.00 | 61.05 | O |
| ATOM | 1 | N | LEU | B | 184 | −11.394 | −48.634 | −40.344 | 1.00 | 60.90 | N |
| ATOM | 2 | CA | LEU | B | 184 | −10.151 | −49.082 | −39.719 | 1.00 | 60.71 | C |
| ATOM | 3 | CB | LEU | B | 184 | −10.130 | −50.608 | −39.576 | 1.00 | 60.75 | C |
| ATOM | 4 | CG | LEU | B | 184 | −10.626 | −51.298 | −38.302 | 1.00 | 60.63 | C |
| ATOM | 5 | CD1 | LEU | B | 184 | −10.247 | −52.774 | −38.346 | 1.00 | 60.23 | C |
| ATOM | 6 | CD2 | LEU | B | 184 | −10.055 | −50.648 | −37.055 | 1.00 | 60.26 | C |
| ATOM | 7 | C | LEU | B | 184 | −8.948 | −48.655 | −40.548 | 1.00 | 60.59 | C |
| ATOM | 8 | O | LEU | B | 184 | −9.008 | −48.633 | −41.776 | 1.00 | 60.73 | O |
| ATOM | 1 | N | ILE | B | 185 | −7.851 | −48.320 | −39.878 | 1.00 | 60.33 | N |
| ATOM | 2 | CA | ILE | B | 185 | −6.619 | −47.996 | −40.573 | 1.00 | 60.09 | C |
| ATOM | 3 | CB | ILE | B | 185 | −6.170 | −46.560 | −40.294 | 1.00 | 59.94 | C |
| ATOM | 4 | CG1 | ILE | B | 185 | −7.327 | −45.592 | −40.540 | 1.00 | 59.84 | C |
| ATOM | 5 | CD1 | ILE | B | 185 | −6.969 | −44.139 | −40.330 | 1.00 | 59.85 | C |
| ATOM | 6 | CG2 | ILE | B | 185 | −4.983 | −46.203 | −41.165 | 1.00 | 59.66 | C |
| ATOM | 7 | C | ILE | B | 185 | −5.539 | −48.964 | −40.129 | 1.00 | 60.22 | C |
| ATOM | 8 | O | ILE | B | 185 | −5.136 | −48.969 | −38.968 | 1.00 | 60.25 | O |
| ATOM | 1 | N | ILE | B | 186 | −5.086 | −49.799 | −41.053 | 1.00 | 60.39 | N |
| ATOM | 2 | CA | ILE | B | 186 | −4.057 | −50.777 | −40.738 | 1.00 | 60.66 | C |
| ATOM | 3 | CB | ILE | B | 186 | −4.285 | −52.100 | −41.483 | 1.00 | 60.60 | C |
| ATOM | 4 | CG1 | ILE | B | 186 | −5.722 | −52.588 | −41.291 | 1.00 | 60.80 | C |
| ATOM | 5 | CD1 | ILE | B | 186 | −6.119 | −52.766 | −39.846 | 1.00 | 60.97 | C |
| ATOM | 6 | CG2 | ILE | B | 186 | −3.308 | −53.141 | −41.002 | 1.00 | 60.56 | C |
| ATOM | 7 | C | ILE | B | 186 | −2.688 | −50.230 | −41.115 | 1.00 | 60.88 | C |
| ATOM | 8 | O | ILE | B | 186 | −2.435 | −49.911 | −42.280 | 1.00 | 60.99 | O |
| ATOM | 1 | N | LEU | B | 187 | −1.812 | −50.107 | −40.126 | 1.00 | 61.05 | N |
| ATOM | 2 | CA | LEU | B | 187 | −0.445 | −49.672 | −40.376 | 1.00 | 61.25 | C |
| ATOM | 3 | CB | LEU | B | 187 | −0.055 | −48.542 | −39.425 | 1.00 | 61.17 | C |
| ATOM | 4 | CG | LEU | B | 187 | −0.336 | −47.107 | −39.873 | 1.00 | 60.76 | C |
| ATOM | 5 | CD1 | LEU | B | 187 | −1.790 | −46.918 | −40.216 | 1.00 | 60.58 | C |
| ATOM | 6 | CD2 | LEU | B | 187 | 0.075 | −46.133 | −38.787 | 1.00 | 60.96 | C |
| ATOM | 7 | C | LEU | B | 187 | 0.520 | −50.850 | −40.234 | 1.00 | 61.68 | C |
| ATOM | 8 | O | LEU | B | 187 | 0.545 | −51.526 | −39.201 | 1.00 | 61.88 | O |
| ATOM | 1 | N | LEU | B | 188 | 1.306 | −51.101 | −41.277 | 1.00 | 61.91 | N |
| ATOM | 2 | CA | LEU | B | 188 | 2.240 | −52.216 | −41.256 | 1.00 | 62.18 | C |
| ATOM | 3 | CB | LEU | B | 188 | 2.152 | −53.033 | −42.542 | 1.00 | 62.05 | C |
| ATOM | 4 | CG | LEU | B | 188 | 0.838 | −53.756 | −42.816 | 1.00 | 62.08 | C |
| ATOM | 5 | CD1 | LEU | B | 188 | 1.125 | −55.091 | −43.478 | 1.00 | 61.97 | C |
| ATOM | 6 | CD2 | LEU | B | 188 | 0.084 | −53.970 | −41.530 | 1.00 | 62.07 | C |
| ATOM | 7 | C | LEU | B | 188 | 3.669 | −51.755 | −41.050 | 1.00 | 62.53 | C |
| ATOM | 8 | O | LEU | B | 188 | 4.128 | −50.801 | −41.678 | 1.00 | 62.71 | O |
| ATOM | 1 | N | PHE | B | 189 | 4.362 | −52.441 | −40.154 | 1.00 | 62.88 | N |
| ATOM | 2 | CA | PHE | B | 189 | 5.797 | −52.305 | −40.021 | 1.00 | 63.25 | C |
| ATOM | 3 | CB | PHE | B | 189 | 6.152 | −51.737 | −38.661 | 1.00 | 63.06 | C |
| ATOM | 4 | CG | PHE | B | 189 | 5.690 | −50.339 | −38.458 | 1.00 | 63.11 | C |
| ATOM | 5 | CD1 | PHE | B | 189 | 4.415 | −50.080 | −37.994 | 1.00 | 63.21 | C |
| ATOM | 6 | CE1 | PHE | B | 189 | 3.987 | −48.781 | −37.802 | 1.00 | 63.18 | C |
| ATOM | 7 | CZ | PHE | B | 189 | 4.841 | −47.727 | −38.078 | 1.00 | 63.21 | C |
| ATOM | 8 | CE2 | PHE | B | 189 | 6.115 | −47.974 | −38.544 | 1.00 | 63.09 | C |
| ATOM | 9 | CD2 | PHE | B | 189 | 6.534 | −49.274 | −38.731 | 1.00 | 63.37 | C |
| ATOM | 10 | C | PHE | B | 189 | 6.332 | −53.707 | −40.128 | 1.00 | 63.65 | C |
| ATOM | 11 | O | PHE | B | 189 | 5.702 | −54.631 | −39.626 | 1.00 | 63.87 | O |
| ATOM | 1 | N | ASP | B | 190 | 7.472 | −53.895 | −40.779 | 1.00 | 64.11 | N |
| ATOM | 2 | CA | ASP | B | 190 | 8.012 | −55.244 | −40.854 | 1.00 | 64.78 | C |
| ATOM | 3 | CB | ASP | B | 190 | 8.204 | −55.712 | −42.308 | 1.00 | 64.95 | C |
| ATOM | 4 | CG | ASP | B | 190 | 9.594 | −55.455 | −42.843 | 1.00 | 65.15 | C |
| ATOM | 5 | OD1 | ASP | B | 190 | 10.067 | −54.305 | −42.750 | 1.00 | 66.38 | O |
| ATOM | 6 | OD2 | ASP | B | 190 | 10.201 | −56.403 | −43.386 | 1.00 | 64.63 | O |
| ATOM | 7 | C | ASP | B | 190 | 9.248 | −55.446 | −39.972 | 1.00 | 65.11 | C |
| ATOM | 8 | O | ASP | B | 190 | 10.233 | −54.713 | −40.073 | 1.00 | 65.07 | O |
| ATOM | 1 | N | ALA | B | 191 | 9.152 | −56.441 | −39.090 | 1.00 | 65.57 | N |
| ATOM | 2 | CA | ALA | B | 191 | 10.175 | −56.747 | −38.090 | 1.00 | 65.98 | C |
| ATOM | 3 | CB | ALA | B | 191 | 9.723 | −57.918 | −37.239 | 1.00 | 66.05 | C |
| ATOM | 4 | C | ALA | B | 191 | 11.499 | −57.076 | −38.740 | 1.00 | 66.33 | C |
| ATOM | 5 | O | ALA | B | 191 | 12.521 | −57.215 | −38.072 | 1.00 | 66.29 | O |
| ATOM | 1 | N | HIS | B | 192 | 11.461 | −57.206 | −40.057 | 1.00 | 66.88 | N |
| ATOM | 2 | CA | HIS | B | 192 | 12.615 | −57.594 | −40.825 | 1.00 | 67.53 | C |
| ATOM | 3 | CB | HIS | B | 192 | 12.141 | −58.253 | −42.110 | 1.00 | 67.72 | C |
| ATOM | 4 | CG | HIS | B | 192 | 13.248 | −58.756 | −42.971 | 1.00 | 68.70 | C |
| ATOM | 5 | ND1 | HIS | B | 192 | 14.167 | −59.684 | −42.529 | 1.00 | 69.77 | N |
| ATOM | 6 | CE1 | HIS | B | 192 | 15.024 | −59.947 | −43.500 | 1.00 | 70.36 | C |
| ATOM | 7 | NE2 | HIS | B | 192 | 14.688 | −59.227 | −44.556 | 1.00 | 71.03 | N |
| ATOM | 8 | CD2 | HIS | B | 192 | 13.579 | −58.474 | −44.251 | 1.00 | 69.62 | C |
| ATOM | 9 | C | HIS | B | 192 | 13.472 | −56.372 | −41.132 | 1.00 | 67.83 | C |
| ATOM | 10 | O | HIS | B | 192 | 14.694 | −56.409 | −40.993 | 1.00 | 68.11 | O |
| ATOM | 1 | N | LYS | B | 193 | 12.819 | −55.290 | −41.547 | 1.00 | 68.10 | N |
| ATOM | 2 | CA | LYS | B | 193 | 13.490 | −54.027 | −41.844 | 1.00 | 68.23 | C |
| ATOM | 3 | CB | LYS | B | 193 | 13.471 | −53.736 | −43.347 | 1.00 | 68.19 | C |
| ATOM | 4 | CG | LYS | B | 193 | 14.585 | −54.383 | −44.153 | 1.00 | 68.42 | C |
| ATOM | 5 | CD | LYS | B | 193 | 14.700 | −53.722 | −45.523 | 1.00 | 68.19 | C |
| ATOM | 6 | CE | LYS | B | 193 | 14.875 | −52.216 | −45.374 | 1.00 | 68.29 | C |
| ATOM | 7 | NZ | LYS | B | 193 | 14.632 | −51.484 | −46.644 | 1.00 | 68.54 | N |
| ATOM | 8 | C | LYS | B | 193 | 12.783 | −52.889 | −41.133 | 1.00 | 68.36 | C |
| ATOM | 9 | O | LYS | B | 193 | 12.009 | −52.156 | −41.750 | 1.00 | 68.44 | O |
| ATOM | 1 | N | LEU | B | 194 | 13.041 | −52.730 | −39.843 | 1.00 | 68.56 | N |
| ATOM | 2 | CA | LEU | B | 194 | 12.400 | −51.653 | −39.102 | 1.00 | 68.79 | C |
| ATOM | 3 | CB | LEU | B | 194 | 12.608 | −51.826 | −37.601 | 1.00 | 68.92 | C |
| ATOM | 4 | CG | LEU | B | 194 | 11.448 | −51.373 | −36.716 | 1.00 | 68.90 | C |
| ATOM | 5 | CD1 | LEU | B | 194 | 12.004 | −50.606 | −35.540 | 1.00 | 69.72 | C |
| ATOM | 6 | CD2 | LEU | B | 194 | 10.462 | −50.514 | −37.478 | 1.00 | 69.07 | C |
| ATOM | 7 | C | LEU | B | 194 | 12.930 | −50.294 | −39.548 | 1.00 | 68.86 | C |
| ATOM | 8 | O | LEU | B | 194 | 14.113 | −49.994 | −39.399 | 1.00 | 68.87 | O |
| ATOM | 1 | N | GLU | B | 195 | 12.041 | −49.479 | −40.102 | 1.00 | 69.01 | N |
| ATOM | 2 | CA | GLU | B | 195 | 12.386 | −48.129 | −40.535 | 1.00 | 69.24 | C |
| ATOM | 3 | CB | GLU | B | 195 | 12.967 | −48.156 | −41.957 | 1.00 | 69.19 | C |
| ATOM | 4 | CG | GLU | B | 195 | 12.565 | −46.994 | −42.859 | 1.00 | 69.68 | C |
| ATOM | 5 | CD | GLU | B | 195 | 13.235 | −45.679 | −42.488 | 1.00 | 70.76 | C |
| ATOM | 6 | OE1 | GLU | B | 195 | 13.283 | −45.345 | −41.286 | 1.00 | 71.25 | O |
| ATOM | 7 | OE2 | GLU | B | 195 | 13.705 | −44.968 | −43.404 | 1.00 | 70.87 | O |
| ATOM | 8 | C | GLU | B | 195 | 11.147 | −47.238 | −40.450 | 1.00 | 69.31 | C |
| ATOM | 9 | O | GLU | B | 195 | 10.061 | −47.641 | −40.867 | 1.00 | 69.43 | O |
| ATOM | 1 | N | ILE | B | 196 | 11.302 | −46.047 | −39.870 | 1.00 | 69.37 | N |
| ATOM | 2 | CA | ILE | B | 196 | 10.214 | −45.058 | −39.835 | 1.00 | 69.32 | C |
| ATOM | 3 | CB | ILE | B | 196 | 9.694 | −44.760 | −38.397 | 1.00 | 69.37 | C |
| ATOM | 4 | CG1 | ILE | B | 196 | 9.222 | −46.045 | −37.716 | 1.00 | 69.31 | C |
| ATOM | 5 | CD1 | ILE | B | 196 | 8.344 | −45.796 | −36.508 | 1.00 | 69.52 | C |
| ATOM | 6 | CG2 | ILE | B | 196 | 8.540 | −43.749 | −38.434 | 1.00 | 69.16 | C |
| ATOM | 7 | C | ILE | B | 196 | 10.627 | −43.763 | −40.542 | 1.00 | 69.13 | C |
| ATOM | 8 | O | LLE | B | 196 | 11.208 | −42.853 | −39.945 | 1.00 | 69.00 | O |
| ATOM | 1 | N | SER | B | 197 | 10.314 | −43.707 | −41.830 | 1.00 | 68.93 | N |
| ATOM | 2 | CA | SER | B | 197 | 10.661 | −42.579 | −42.668 | 1.00 | 68.80 | C |
| ATOM | 3 | CB | SER | B | 197 | 10.288 | −42.892 | −44.112 | 1.00 | 68.89 | C |
| ATOM | 4 | OG | SER | B | 197 | 8.916 | −43.236 | −44.204 | 1.00 | 68.92 | O |
| ATOM | 5 | C | SER | B | 197 | 9.958 | −41.299 | −42.240 | 1.00 | 68.69 | C |
| ATOM | 6 | O | SER | B | 197 | 8.944 | −41.331 | −41.538 | 1.00 | 68.50 | O |
| ATOM | 1 | N | ASP | B | 198 | 10.512 | −40.174 | −42.682 | 1.00 | 68.61 | N |
| ATOM | 2 | CA | ASP | B | 198 | 9.921 | −38.869 | −42.444 | 1.00 | 68.54 | C |
| ATOM | 3 | CB | ASP | B | 198 | 10.792 | −37.785 | −43.081 | 1.00 | 68.63 | C |
| ATOM | 4 | CG | ASP | B | 198 | 10.209 | −36.394 | −42.918 | 1.00 | 69.32 | C |
| ATOM | 5 | OD1 | ASP | B | 198 | 9.211 | −36.245 | −42.179 | 1.00 | 69.90 | O |
| ATOM | 6 | OD2 | ASP | B | 198 | 10.753 | −35.445 | −43.529 | 1.00 | 70.07 | O |
| ATOM | 7 | C | ASP | B | 198 | 8.504 | −38.830 | −43.017 | 1.00 | 68.35 | C |
| ATOM | 8 | O | ASP | B | 198 | 7.541 | −38.466 | −42.323 | 1.00 | 68.12 | O |
| ATOM | 1 | N | GLU | B | 199 | 8.384 | −39.220 | −44.283 | 1.00 | 68.16 | N |
| ATOM | 2 | CA | GLU | B | 199 | 7.094 | −39.264 | −44.957 | 1.00 | 68.12 | C |
| ATOM | 3 | CB | GLU | B | 199 | 7.244 | −39.928 | −46.325 | 1.00 | 68.13 | C |
| ATOM | 4 | CG | GLU | B | 199 | 5.973 | −39.956 | −47.166 | 1.00 | 68.49 | C |
| ATOM | 5 | CD | GLU | B | 199 | 6.217 | −40.500 | −48.568 | 1.00 | 68.60 | C |
| ATOM | 6 | OE1 | GLU | B | 199 | 7.109 | −39.966 | −49.265 | 1.00 | 69.43 | O |
| ATOM | 7 | OE2 | GLU | B | 199 | 5.517 | −41.456 | −48.975 | 1.00 | 68.91 | O |
| ATOM | 8 | C | GLU | B | 199 | 6.050 | −39.994 | −44.109 | 1.00 | 67.92 | C |
| ATOM | 9 | O | GLU | B | 199 | 4.890 | −39.567 | −44.022 | 1.00 | 67.83 | O |
| ATOM | 1 | N | PHE | B | 200 | 6.466 | −41.082 | −43.465 | 1.00 | 67.72 | N |
| ATOM | 2 | CA | PHE | B | 200 | 5.553 | −41.851 | −42.628 | 1.00 | 67.53 | C |
| ATOM | 3 | CB | PHE | B | 200 | 6.213 | −43.134 | −42.130 | 1.00 | 67.63 | C |
| ATOM | 4 | CG | PHE | B | 200 | 5.288 | −44.319 | −42.102 | 1.00 | 67.69 | C |
| ATOM | 5 | CD1 | PHE | B | 200 | 5.610 | −45.482 | −42.781 | 1.00 | 67.87 | C |
| ATOM | 6 | CE1 | PHE | B | 200 | 4.758 | −46.574 | −42.754 | 1.00 | 68.11 | C |
| ATOM | 7 | CZ | PHE | B | 200 | 3.566 | −46.505 | −42.055 | 1.00 | 67.82 | C |
| ATOM | 8 | CE2 | PHE | B | 200 | 3.231 | −45.351 | −41.384 | 1.00 | 67.56 | C |
| ATOM | 9 | CD2 | PHE | B | 200 | 4.089 | −44.267 | −41.409 | 1.00 | 67.81 | C |
| ATOM | 10 | C | PHE | B | 200 | 5.045 | −41.020 | −41.451 | 1.00 | 67.43 | C |
| ATOM | 11 | O | PHE | B | 200 | 3.840 | −40.823 | −41.302 | 1.00 | 67.23 | O |
| ATOM | 1 | N | SER | B | 201 | 5.959 | −40.522 | −40.621 | 1.00 | 67.37 | N |
| ATOM | 2 | CA | SER | B | 201 | 5.560 | −39.690 | −39.484 | 1.00 | 67.44 | C |
| ATOM | 3 | CB | SER | B | 201 | 6.771 | −39.271 | −38.644 | 1.00 | 67.44 | C |
| ATOM | 4 | OG | SER | B | 201 | 7.579 | −38.337 | −39.332 | 1.00 | 67.77 | O |
| ATOM | 5 | C | SER | B | 201 | 4.742 | −38.471 | −39.927 | 1.00 | 67.32 | C |
| ATOM | 6 | O | SER | B | 201 | 3.934 | −37.939 | −39.157 | 1.00 | 67.15 | O |
| ATOM | 1 | N | GLU | B | 202 | 4.954 | −38.033 | −41.167 | 1.00 | 67.40 | N |
| ATOM | 2 | CA | GLU | B | 202 | 4.076 | −37.031 | −41.770 | 1.00 | 67.29 | C |
| ATOM | 3 | CB | GLU | B | 202 | 4.615 | −36.581 | −43.129 | 1.00 | 67.36 | C |
| ATOM | 4 | CG | GLU | B | 202 | 5.525 | −35.366 | −43.080 | 1.00 | 67.70 | C |
| ATOM | 5 | CD | GLU | B | 202 | 4.825 | −34.096 | −43.527 | 1.00 | 68.43 | C |
| ATOM | 6 | OE1 | GLU | B | 202 | 3.901 | −34.193 | −44.363 | 1.00 | 68.71 | O |
| ATOM | 7 | OE2 | GLU | B | 202 | 5.204 | −33.000 | −43.055 | 1.00 | 68.82 | O |
| ATOM | 8 | C | GLU | B | 202 | 2.668 | −37.602 | −41.926 | 1.00 | 67.11 | C |
| ATOM | 9 | O | GLU | B | 202 | 1.676 | −36.963 | −41.551 | 1.00 | 66.97 | O |
| ATOM | 1 | N | ALA | B | 203 | 2.584 | −38.811 | −42.472 | 1.00 | 67.08 | N |
| ATOM | 2 | CA | ALA | B | 203 | 1.294 | −39.491 | −42.602 | 1.00 | 66.87 | C |
| ATOM | 3 | CB | ALA | B | 203 | 1.470 | −40.842 | −43.288 | 1.00 | 66.88 | C |
| ATOM | 4 | C | ALA | B | 203 | 0.580 | −39.656 | −41.250 | 1.00 | 66.81 | C |
| ATOM | 5 | O | ALA | B | 203 | −0.610 | −39.327 | −41.112 | 1.00 | 66.36 | O |
| ATOM | 1 | N | ILE | B | 204 | 1.311 | −40.164 | −40.256 | 1.00 | 66.84 | N |
| ATOM | 2 | CA | ILE | B | 204 | 0.764 | −40.312 | −38.906 | 1.00 | 66.68 | C |
| ATOM | 3 | CB | ILE | B | 204 | 1.734 | −41.034 | −37.958 | 1.00 | 66.55 | C |
| ATOM | 4 | CG1 | ILE | B | 204 | 1.908 | −42.485 | −38.390 | 1.00 | 66.77 | C |
| ATOM | 5 | CD1 | ILE | B | 204 | 3.223 | −42.763 | −39.062 | 1.00 | 67.14 | C |
| ATOM | 6 | CG2 | ILE | B | 204 | 1.218 | −40.998 | −36.538 | 1.00 | 66.27 | C |
| ATOM | 7 | C | ILE | B | 204 | 0.375 | −38.957 | −38.316 | 1.00 | 66.71 | C |
| ATOM | 8 | O | ILE | B | 204 | −0.602 | −38.850 | −37.571 | 1.00 | 66.75 | O |
| ATOM | 1 | N | GLY | B | 205 | 1.141 | −37.922 | −38.648 | 1.00 | 66.74 | N |
| ATOM | 2 | CA | GLY | B | 205 | 0.737 | −36.565 | −38.308 | 1.00 | 66.51 | C |
| ATOM | 3 | C | GLY | B | 205 | −0.615 | −36.275 | −38.937 | 1.00 | 66.38 | C |
| ATOM | 4 | O | GLY | B | 205 | −1.457 | −35.597 | −38.342 | 1.00 | 66.39 | O |
| ATOM | 1 | N | ALA | B | 206 | −0.826 | −36.806 | −40.140 | 1.00 | 66.29 | N |
| ATOM | 2 | CA | ALA | B | 206 | −2.073 | −36.575 | −40.877 | 1.00 | 65.87 | C |
| ATOM | 3 | CB | ALA | B | 206 | −1.866 | −36.813 | −42.368 | 1.00 | 65.95 | C |
| ATOM | 4 | C | ALA | B | 206 | −3.243 | −37.407 | −40.365 | 1.00 | 65.58 | C |
| ATOM | 5 | O | ALA | B | 206 | −4.400 | −37.072 | −40.603 | 1.00 | 65.48 | O |
| ATOM | 1 | N | LEU | B | 207 | −2.946 | −38.499 | −39.673 | 1.00 | 65.49 | N |
| ATOM | 2 | CA | LEU | B | 207 | −4.006 | −39.322 | −39.088 | 1.00 | 65.08 | C |
| ATOM | 3 | CB | LEU | B | 207 | −3.540 | −40.769 | −38.935 | 1.00 | 65.03 | C |
| ATOM | 4 | CG | LEU | B | 207 | −3.898 | −41.743 | −40.060 | 1.00 | 65.05 | C |
| ATOM | 5 | CD1 | LEU | B | 207 | −3.971 | −41.051 | −41.411 | 1.00 | 65.29 | C |
| ATOM | 6 | CD2 | LEU | B | 207 | −2.898 | −42.885 | −40.101 | 1.00 | 65.61 | C |
| ATOM | 7 | C | LEU | B | 207 | −4.509 | −38.785 | −37.747 | 1.00 | 64.97 | C |
| ATOM | 8 | O | LEU | B | 207 | −5.404 | −39.364 | −37.133 | 1.00 | 64.88 | O |
| ATOM | 1 | N | ARG | B | 208 | −3.933 | −37.674 | −37.301 | 1.00 | 64.92 | N |
| ATOM | 2 | CA | ARG | B | 208 | −4.271 | −37.083 | −36.004 | 1.00 | 64.83 | C |
| ATOM | 3 | CB | ARG | B | 208 | −3.525 | −35.751 | −35.819 | 1.00 | 64.82 | C |
| ATOM | 4 | CG | ARG | B | 208 | −4.134 | −34.791 | −34.796 | 1.00 | 64.69 | C |
| ATOM | 5 | CD | ARG | B | 208 | −3.025 | −34.039 | −34.043 | 1.00 | 64.59 | C |
| ATOM | 6 | NE | ARG | B | 208 | −3.246 | −32.581 | −33.961 | 1.00 | 64.85 | N |
| ATOM | 7 | CZ | ARG | B | 208 | −4.343 | −31.934 | −34.378 | 1.00 | 64.78 | C |
| ATOM | 8 | NH1 | ARG | B | 208 | −5.373 | −32.594 | −34.925 | 1.00 | 65.02 | N |
| ATOM | 9 | NH2 | ARG | B | 208 | −4.407 | −30.609 | −34.238 | 1.00 | 64.48 | N |
| ATOM | 10 | C | ARG | B | 208 | −5.779 | −36.913 | −35.789 | 1.00 | 64.76 | C |
| ATOM | 11 | O | ARG | B | 208 | −6.493 | −36.423 | −36.665 | 1.00 | 64.84 | O |
| ATOM | 1 | N | GLY | B | 209 | −6.253 | −37.327 | −34.617 | 1.00 | 64.60 | N |
| ATOM | 2 | CA | GLY | B | 209 | −7.663 | −37.188 | −34.261 | 1.00 | 64.23 | C |
| ATOM | 3 | C | GLY | B | 209 | −8.447 | −38.455 | −34.531 | 1.00 | 63.99 | C |
| ATOM | 4 | O | GLY | B | 209 | −9.631 | −38.553 | −34.206 | 1.00 | 63.84 | O |
| ATOM | 1 | N | HIS | B | 210 | −7.781 | −39.431 | −35.134 | 1.00 | 63.86 | N |
| ATOM | 2 | CA | HIS | B | 210 | −8.432 | −40.685 | −35.475 | 1.00 | 63.78 | C |
| ATOM | 3 | CB | HIS | B | 210 | −8.683 | −40.769 | −36.983 | 1.00 | 63.93 | C |
| ATOM | 4 | CG | HIS | B | 210 | −9.689 | −39.779 | −37.482 | 1.00 | 64.53 | C |
| ATOM | 5 | ND1 | HIS | B | 210 | −9.380 | −38.807 | −38.409 | 1.00 | 65.05 | N |
| ATOM | 6 | CE1 | HIS | B | 210 | −10.455 | −38.079 | −38.655 | 1.00 | 65.36 | C |
| ATOM | 7 | NE2 | HIS | B | 210 | −11.449 | −38.540 | −37.916 | 1.00 | 65.43 | N |
| ATOM | 8 | CD2 | HIS | B | 210 | −10.996 | −39.602 | −37.172 | 1.00 | 65.12 | C |
| ATOM | 9 | C | HIS | B | 210 | −7.632 | −41.891 | −35.005 | 1.00 | 63.46 | C |
| ATOM | 10 | O | HIS | B | 210 | −7.808 | −42.996 | −35.517 | 1.00 | 63.56 | O |
| ATOM | 1 | N | GLU | B | 211 | −6.761 | −41.682 | −34.023 | 1.00 | 63.02 | N |
| ATOM | 2 | CA | GLU | B | 211 | −5.938 | −42.767 | −33.510 | 1.00 | 62.65 | C |
| ATOM | 3 | CB | GLU | B | 211 | −5.082 | −42.298 | −32.335 | 1.00 | 62.67 | C |
| ATOM | 4 | CG | GLU | B | 211 | −5.511 | −40.968 | −31.750 | 1.00 | 63.51 | C |
| ATOM | 5 | CD | GLU | B | 211 | −4.739 | −39.803 | −32.341 | 1.00 | 64.41 | C |
| ATOM | 6 | OE1 | GLU | B | 211 | −5.237 | −38.656 | −32.290 | 1.00 | 64.72 | O |
| ATOM | 7 | OE2 | GLU | B | 211 | −3.626 | −40.038 | −32.856 | 1.00 | 64.94 | O |
| ATOM | 8 | C | GLU | B | 211 | −6.761 | −43.999 | −33.127 | 1.00 | 62.27 | C |
| ATOM | 9 | O | GLU | B | 211 | −6.280 | −45.128 | −33.235 | 1.00 | 62.45 | O |
| ATOM | 1 | N | ASP | B | 212 | −8.002 | −43.786 | −32.701 | 1.00 | 61.63 | N |
| ATOM | 2 | CA | ASP | B | 212 | −8.856 | −44.897 | −32.283 | 1.00 | 61.13 | C |
| ATOM | 3 | CB | ASP | B | 212 | −10.158 | −44.381 | −31.677 | 1.00 | 61.30 | C |
| ATOM | 4 | CG | ASP | B | 212 | −10.999 | −43.615 | −32.676 | 1.00 | 62.39 | C |
| ATOM | 5 | OD1 | ASP | B | 212 | −10.792 | −42.385 | −32.807 | 1.00 | 63.83 | O |
| ATOM | 6 | OD2 | ASP | B | 212 | −11.874 | −44.239 | −33.322 | 1.00 | 63.08 | O |
| ATOM | 7 | C | ASP | B | 212 | −9.164 | −45.858 | −33.428 | 1.00 | 60.47 | C |
| ATOM | 8 | O | ASP | B | 212 | −9.609 | −46.983 | −33.202 | 1.00 | 60.32 | O |
| ATOM | 1 | N | LYS | B | 213 | −8.927 | −45.410 | −34.654 | 1.00 | 59.79 | N |
| ATOM | 2 | CA | LYS | B | 213 | −9.201 | −46.226 | −35.825 | 1.00 | 59.18 | C |
| ATOM | 3 | CB | LYS | B | 213 | −9.712 | −45.361 | −36.974 | 1.00 | 59.14 | C |
| ATOM | 4 | CG | LYS | B | 213 | −11.066 | −44.724 | −36.747 | 1.00 | 59.05 | C |
| ATOM | 5 | CD | LYS | B | 213 | −11.580 | −44.131 | −38.052 | 1.00 | 59.11 | C |
| ATOM | 6 | CE | LYS | B | 213 | −12.808 | −43.262 | −37.850 | 1.00 | 58.78 | C |
| ATOM | 7 | NZ | LYS | B | 213 | −13.143 | −42.542 | −39.105 | 1.00 | 58.30 | N |
| ATOM | 8 | C | LYS | B | 213 | −7.956 | −46.962 | −36.282 | 1.00 | 58.86 | C |
| ATOM | 9 | O | LYS | B | 213 | −7.964 | −47.627 | −37.315 | 1.00 | 58.84 | O |
| ATOM | 1 | N | ILE | B | 214 | −6.884 | −46.851 | −35.511 | 1.00 | 58.47 | N |
| ATOM | 2 | CA | ILE | B | 214 | −5.599 | −47.377 | −35.948 | 1.00 | 58.17 | C |
| ATOM | 3 | CB | ILE | B | 214 | −4.467 | −46.389 | −35.634 | 1.00 | 58.12 | C |
| ATOM | 4 | CG1 | ILE | B | 214 | −4.670 | −45.098 | −36.424 | 1.00 | 58.10 | C |
| ATOM | 5 | CD1 | ILE | B | 214 | −3.652 | −44.022 | −36.118 | 1.00 | 58.21 | C |
| ATOM | 6 | CG2 | ILE | B | 214 | −3.122 | −47.009 | −35.959 | 1.00 | 58.27 | C |
| ATOM | 7 | C | ILE | B | 214 | −5.245 | −48.735 | −35.352 | 1.00 | 57.94 | C |
| ATOM | 8 | O | ILE | B | 214 | −5.231 | −48.908 | −34.137 | 1.00 | 58.08 | O |
| ATOM | 1 | N | ARG | B | 215 | −4.960 | −49.698 | −36.219 | 1.00 | 57.63 | N |
| ATOM | 2 | CA | ARG | B | 215 | −4.361 | −50.946 | −35.782 | 1.00 | 57.37 | C |
| ATOM | 3 | CB | ARG | B | 215 | −5.175 | −52.152 | −36.242 | 1.00 | 57.18 | C |
| ATOM | 4 | CG | ARG | B | 215 | −6.611 | −52.145 | −35.811 | 1.00 | 56.81 | C |
| ATOM | 5 | CD | ARG | B | 215 | −6.766 | −51.984 | −34.314 | 1.00 | 56.23 | C |
| ATOM | 6 | NE | ARG | B | 215 | −8.176 | −51.817 | −33.978 | 1.00 | 56.42 | N |
| ATOM | 7 | CZ | ARG | B | 215 | −8.802 | −50.645 | −33.950 | 1.00 | 56.18 | C |
| ATOM | 8 | NH1 | ARG | B | 215 | −8.140 | −49.527 | −34.219 | 1.00 | 56.41 | N |
| ATOM | 9 | NH2 | ARG | B | 215 | −10.090 | −50.591 | −33.649 | 1.00 | 55.83 | N |
| ATOM | 10 | C | ARG | B | 215 | −2.972 | −51.029 | −36.380 | 1.00 | 57.38 | C |
| ATOM | 11 | O | ARG | B | 215 | −2.820 | −51.179 | −37.596 | 1.00 | 57.59 | O |
| ATOM | 1 | N | VAL | B | 216 | −1.961 | −50.913 | −35.526 | 1.00 | 57.17 | N |
| ATOM | 2 | CA | VAL | B | 216 | −0.575 | −51.040 | −35.949 | 1.00 | 56.79 | C |
| ATOM | 3 | CB | VAL | B | 216 | 0.346 | −50.313 | −34.971 | 1.00 | 56.68 | C |
| ATOM | 4 | CG1 | VAL | B | 216 | 1.797 | −50.608 | −35.293 | 1.00 | 56.77 | C |
| ATOM | 5 | CG2 | VAL | B | 216 | 0.069 | −48.823 | −34.997 | 1.00 | 56.69 | C |
| ATOM | 6 | C | VAL | B | 216 | −0.188 | −52.506 | −35.942 | 1.00 | 56.67 | C |
| ATOM | 7 | O | VAL | B | 216 | −0.159 | −53.124 | −34.884 | 1.00 | 56.91 | O |
| ATOM | 1 | N | VAL | B | 217 | 0.105 | −53.079 | −37.105 | 1.00 | 56.39 | N |
| ATOM | 2 | CA | VAL | B | 217 | 0.520 | −54.478 | −37.123 | 1.00 | 56.08 | C |
| ATOM | 3 | CB | VAL | B | 217 | −0.406 | −55.381 | −37.978 | 1.00 | 56.09 | C |
| ATOM | 4 | CG1 | VAL | B | 217 | −1.604 | −54.600 | −38.471 | 1.00 | 55.83 | C |
| ATOM | 5 | CG2 | VAL | B | 217 | 0.352 | −56.004 | −39.138 | 1.00 | 56.09 | C |
| ATOM | 6 | C | VAL | B | 217 | 1.971 | −54.632 | −37.548 | 1.00 | 56.01 | C |
| ATOM | 7 | O | VAL | B | 217 | 2.388 | −54.152 | −38.608 | 1.00 | 56.14 | O |
| ATOM | 1 | N | LEU | B | 218 | 2.734 | −55.297 | −36.688 | 1.00 | 55.76 | N |
| ATOM | 2 | CA | LEU | B | 218 | 4.136 | −55.593 | −36.933 | 1.00 | 55.35 | C |
| ATOM | 3 | CB | LEU | B | 218 | 4.878 | −55.634 | −35.604 | 1.00 | 55.27 | C |
| ATOM | 4 | CG | LEU | B | 218 | 6.311 | −56.137 | −35.529 | 1.00 | 55.27 | C |
| ATOM | 5 | CD1 | LEU | B | 218 | 7.252 | −55.189 | −36.244 | 1.00 | 55.39 | C |
| ATOM | 6 | CD2 | LEU | B | 218 | 6.683 | −56.270 | −34.066 | 1.00 | 55.68 | C |
| ATOM | 7 | C | LEU | B | 218 | 4.239 | −56.926 | −37.670 | 1.00 | 55.33 | C |
| ATOM | 8 | O | LEU | B | 218 | 4.112 | −58.006 | −37.084 | 1.00 | 55.26 | O |
| ATOM | 1 | N | ASN | B | 219 | 4.460 | −56.830 | −38.972 | 1.00 | 55.33 | N |
| ATOM | 2 | CA | ASN | B | 219 | 4.411 | −57.970 | −39.865 | 1.00 | 55.16 | C |
| ATOM | 3 | CB | ASN | B | 219 | 4.108 | −57.479 | −41.276 | 1.00 | 55.29 | C |
| ATOM | 4 | CG | ASN | B | 219 | 3.409 | −58.516 | −42.109 | 1.00 | 55.94 | C |
| ATOM | 5 | OD1 | ASN | B | 219 | 3.026 | −59.575 | −41.601 | 1.00 | 56.02 | O |
| ATOM | 6 | ND2 | ASN | B | 219 | 3.234 | −58.225 | −43.403 | 1.00 | 56.54 | N |
| ATOM | 7 | C | ASN | B | 219 | 5.693 | −58.789 | −39.875 | 1.00 | 55.04 | C |
| ATOM | 8 | O | ASN | B | 219 | 6.704 | −58.388 | −39.301 | 1.00 | 54.74 | O |
| ATOM | 1 | N | LYS | B | 220 | 5.632 | −59.937 | −40.546 | 1.00 | 55.21 | N |
| ATOM | 2 | CA | LYS | B | 220 | 6.756 | −60.871 | −40.681 | 1.00 | 55.41 | C |
| ATOM | 3 | CB | LYS | B | 220 | 7.602 | −60.553 | −41.925 | 1.00 | 55.43 | C |
| ATOM | 4 | CG | LYS | B | 220 | 7.598 | −59.096 | −42.343 | 1.00 | 55.52 | C |
| ATOM | 5 | CD | LYS | B | 220 | 7.343 | −58.922 | −43.840 | 1.00 | 55.10 | C |
| ATOM | 6 | CE | LYS | B | 220 | 8.608 | −58.547 | −44.594 | 1.00 | 55.18 | C |
| ATOM | 7 | NZ | LYS | B | 220 | 8.297 | −57.685 | −45.768 | 1.00 | 54.26 | N |
| ATOM | 8 | C | LYS | B | 220 | 7.627 | −61.022 | −39.437 | 1.00 | 55.67 | C |
| ATOM | 9 | O | LYS | B | 220 | 8.848 | −61.107 | −39.531 | 1.00 | 55.78 | O |
| ATOM | 1 | N | ALA | B | 221 | 6.987 | −61.082 | −38.273 | 1.00 | 56.06 | N |
| ATOM | 2 | CA | ALA | B | 221 | 7.691 | −61.285 | −37.011 | 1.00 | 56.27 | C |
| ATOM | 3 | CB | ALA | B | 221 | 6.764 | −61.039 | −35.842 | 1.00 | 56.15 | C |
| ATOM | 4 | C | ALA | B | 221 | 8.259 | −62.689 | −36.932 | 1.00 | 56.45 | C |
| ATOM | 5 | O | ALA | B | 221 | 9.042 | −62.996 | −36.042 | 1.00 | 56.60 | O |
| ATOM | 1 | N | ASP | B | 222 | 7.864 | −63.540 | −37.869 | 1.00 | 56.79 | N |
| ATOM | 2 | CA | ASP | B | 222 | 8.275 | −64.932 | −37.840 | 1.00 | 57.20 | C |
| ATOM | 3 | CB | ASP | B | 222 | 7.246 | −65.817 | −38.537 | 1.00 | 57.26 | C |
| ATOM | 4 | CG | ASP | B | 222 | 7.446 | −65.865 | −40.042 | 1.00 | 58.12 | C |
| ATOM | 5 | OD1 | ASP | B | 222 | 7.687 | −66.978 | −40.571 | 1.00 | 58.75 | O |
| ATOM | 6 | OD2 | ASP | B | 222 | 7.383 | −64.791 | −40.692 | 1.00 | 58.98 | O |
| ATOM | 7 | C | ASP | B | 222 | 9.609 | −65.106 | −38.530 | 1.00 | 57.37 | C |
| ATOM | 8 | O | ASP | B | 222 | 10.166 | −66.205 | −38.549 | 1.00 | 57.48 | O |
| ATOM | 1 | N | MET | B | 223 | 10.120 | −64.038 | −39.124 | 1.00 | 57.52 | N |
| ATOM | 2 | CA | MET | B | 223 | 11.365 | −64.182 | −39.853 | 1.00 | 57.98 | C |
| ATOM | 3 | CB | MET | B | 223 | 11.214 | −63.751 | −41.312 | 1.00 | 57.98 | C |
| ATOM | 4 | CG | MET | B | 223 | 11.104 | −62.271 | −41.558 | 1.00 | 58.43 | C |
| ATOM | 5 | SD | MET | B | 223 | 11.298 | −61.968 | −43.330 | 1.00 | 59.28 | S |
| ATOM | 6 | CE | MET | B | 223 | 12.766 | −62.938 | −43.695 | 1.00 | 59.20 | C |
| ATOM | 7 | C | MET | B | 223 | 12.525 | −63.489 | −39.163 | 1.00 | 57.63 | C |
| ATOM | 8 | O | MET | B | 223 | 13.437 | −62.975 | −39.807 | 1.00 | 57.84 | O |
| ATOM | 1 | N | VAL | B | 224 | 12.495 | −63.514 | −37.838 | 1.00 | 57.30 | N |
| ATOM | 2 | CA | VAL | B | 224 | 13.536 | −62.896 | −37.049 | 1.00 | 56.98 | C |
| ATOM | 3 | CB | VAL | B | 224 | 13.260 | −61.386 | −36.882 | 1.00 | 56.86 | C |
| ATOM | 4 | CG1 | VAL | B | 224 | 12.529 | −61.108 | −35.585 | 1.00 | 56.82 | C |
| ATOM | 5 | CG2 | VAL | B | 224 | 14.547 | −60.602 | −36.947 | 1.00 | 56.98 | C |
| ATOM | 6 | C | VAL | B | 224 | 13.608 | −63.608 | −35.700 | 1.00 | 56.86 | C |
| ATOM | 7 | O | VAL | B | 224 | 12.583 | −63.957 | −35.119 | 1.00 | 56.87 | O |
| ATOM | 1 | N | GLU | B | 225 | 14.818 | −63.856 | −35.218 | 1.00 | 56.71 | N |
| ATOM | 2 | CA | GLU | B | 225 | 14.987 | −64.536 | −33.946 | 1.00 | 56.69 | C |
| ATOM | 3 | CB | GLU | B | 225 | 16.468 | −64.682 | −33.627 | 1.00 | 57.01 | C |
| ATOM | 4 | CG | GLU | B | 225 | 16.782 | −65.935 | −32.848 | 1.00 | 58.92 | C |
| ATOM | 5 | CD | GLU | B | 225 | 16.999 | −67.130 | −33.754 | 1.00 | 61.73 | C |
| ATOM | 6 | OE1 | GLU | B | 225 | 16.006 | −67.812 | −34.110 | 1.00 | 62.80 | O |
| ATOM | 7 | OE2 | GLU | B | 225 | 18.171 | −67.378 | −34.122 | 1.00 | 63.64 | O |
| ATOM | 8 | C | GLU | B | 225 | 14.287 | −63.731 | −32.854 | 1.00 | 56.21 | C |
| ATOM | 9 | O | GLU | B | 225 | 14.195 | −62.512 | −32.948 | 1.00 | 56.09 | O |
| ATOM | 1 | N | THR | B | 226 | 13.808 | −64.399 | −31.812 | 1.00 | 55.84 | N |
| ATOM | 2 | CA | THR | B | 226 | 12.913 | −63.730 | −30.861 | 1.00 | 55.74 | C |
| ATOM | 3 | CB | THR | B | 226 | 12.207 | −64.715 | −29.894 | 1.00 | 55.64 | C |
| ATOM | 4 | OG1 | THR | B | 226 | 12.183 | −64.152 | −28.577 | 1.00 | 55.50 | O |
| ATOM | 5 | CG2 | THR | B | 226 | 12.920 | −66.054 | −29.862 | 1.00 | 56.27 | C |
| ATOM | 6 | C | THR | B | 226 | 13.473 | −62.487 | −30.127 | 1.00 | 55.65 | C |
| ATOM | 7 | O | THR | B | 226 | 12.854 | −61.416 | −30.168 | 1.00 | 55.72 | O |
| ATOM | 1 | N | GLN | B | 227 | 14.618 | −62.618 | −29.457 | 1.00 | 55.48 | N |
| ATOM | 2 | CA | GLN | B | 227 | 15.255 | −61.451 | −28.828 | 1.00 | 55.18 | C |
| ATOM | 3 | CB | GLN | B | 227 | 16.718 | −61.737 | −28.508 | 1.00 | 55.14 | C |
| ATOM | 4 | CG | GLN | B | 227 | 16.996 | −62.165 | −27.086 | 1.00 | 54.85 | C |
| ATOM | 5 | CD | GLN | B | 227 | 18.445 | −62.543 | −26.892 | 1.00 | 54.43 | C |
| ATOM | 6 | OE1 | GLN | B | 227 | 19.045 | −63.201 | −27.747 | 1.00 | 53.99 | O |
| ATOM | 7 | NE2 | GLN | B | 227 | 19.023 | −62.124 | −25.772 | 1.00 | 54.27 | N |
| ATOM | 8 | C | GLN | B | 227 | 15.191 | −60.245 | −29.757 | 1.00 | 55.20 | C |
| ATOM | 9 | O | GLN | B | 227 | 14.762 | −59.151 | −29.362 | 1.00 | 55.06 | O |
| ATOM | 1 | N | GLN | B | 228 | 15.630 | −60.463 | −30.995 | 1.00 | 55.21 | N |
| ATOM | 2 | CA | GLN | B | 228 | 15.591 | −59.450 | −32.035 | 1.00 | 55.26 | C |
| ATOM | 3 | CB | GLN | B | 228 | 16.104 | −60.039 | −33.345 | 1.00 | 55.25 | C |
| ATOM | 4 | CG | GLN | B | 228 | 15.985 | −59.113 | −34.533 | 1.00 | 55.38 | C |
| ATOM | 5 | CD | GLN | B | 228 | 16.759 | −57.832 | −34.349 | 1.00 | 55.86 | C |
| ATOM | 6 | OE1 | GLN | B | 228 | 17.766 | −57.794 | −33.640 | 1.00 | 56.42 | O |
| ATOM | 7 | NE2 | GLN | B | 228 | 16.295 | −56.769 | −34.991 | 1.00 | 56.22 | N |
| ATOM | 8 | C | GLN | B | 228 | 14.180 | −58.880 | −32.213 | 1.00 | 55.39 | C |
| ATOM | 9 | O | GLN | B | 228 | 13.996 | −57.660 | −32.302 | 1.00 | 55.39 | O |
| ATOM | 1 | N | LEU | B | 229 | 13.184 | −59.761 | −32.256 | 1.00 | 55.35 | N |
| ATOM | 2 | CA | LEU | B | 229 | 11.793 | −59.320 | −32.292 | 1.00 | 55.42 | C |
| ATOM | 3 | CB | LEU | B | 229 | 10.836 | −60.506 | −32.164 | 1.00 | 55.40 | C |
| ATOM | 4 | CG | LEU | B | 229 | 9.514 | −60.430 | −32.927 | 1.00 | 55.19 | C |
| ATOM | 5 | CD1 | LEU | B | 229 | 8.475 | −61.296 | −32.239 | 1.00 | 55.13 | C |
| ATOM | 6 | CD2 | LEU | B | 229 | 9.024 | −59.001 | −33.040 | 1.00 | 54.53 | C |
| ATOM | 7 | C | LEU | B | 229 | 11.545 | −58.334 | −31.155 | 1.00 | 55.47 | C |
| ATOM | 8 | O | LEU | B | 229 | 11.095 | −57.209 | −31.390 | 1.00 | 55.55 | O |
| ATOM | 1 | N | MET | B | 230 | 11.854 | −58.752 | −29.928 | 1.00 | 55.41 | N |
| ATOM | 2 | CA | MET | B | 230 | 11.723 | −57.862 | −28.775 | 1.00 | 55.26 | C |
| ATOM | 3 | CB | MET | B | 230 | 12.318 | −58.497 | −27.519 | 1.00 | 55.44 | C |
| ATOM | 4 | CG | MET | B | 230 | 11.610 | −59.763 | −27.089 | 1.00 | 55.41 | C |
| ATOM | 5 | SD | MET | B | 230 | 9.837 | −59.494 | −26.920 | 1.00 | 56.86 | S |
| ATOM | 6 | CE | MET | B | 230 | 9.228 | −61.082 | −27.495 | 1.00 | 56.24 | C |
| ATOM | 7 | C | MET | B | 230 | 12.389 | −56.523 | −29.053 | 1.00 | 55.18 | C |
| ATOM | 8 | O | MET | B | 230 | 11.804 | −55.459 | −28.819 | 1.00 | 55.12 | O |
| ATOM | 1 | N | ARG | B | 231 | 13.612 | −56.581 | −29.567 | 1.00 | 55.12 | N |
| ATOM | 2 | CA | ARG | B | 231 | 14.336 | −55.368 | −29.936 | 1.00 | 55.21 | C |
| ATOM | 3 | CB | ARG | B | 231 | 15.686 | −55.730 | −30.553 | 1.00 | 55.10 | C |
| ATOM | 4 | CG | ARG | B | 231 | 16.874 | −55.530 | −29.624 | 1.00 | 55.70 | C |
| ATOM | 5 | CD | ARG | B | 231 | 17.000 | −56.597 | −28.551 | 1.00 | 56.51 | C |
| ATOM | 6 | NE | ARG | B | 231 | 16.166 | −56.309 | −27.391 | 1.00 | 57.65 | N |
| ATOM | 7 | CZ | ARG | B | 231 | 16.040 | −57.120 | −26.346 | 1.00 | 57.94 | C |
| ATOM | 8 | NH1 | ARG | B | 231 | 16.704 | −58.268 | −26.313 | 1.00 | 57.78 | N |
| ATOM | 9 | NH2 | ARG | B | 231 | 15.248 | −56.782 | −25.337 | 1.00 | 58.31 | N |
| ATOM | 10 | C | ARG | B | 231 | 13.531 | −54.457 | −30.878 | 1.00 | 55.08 | C |
| ATOM | 11 | O | ARG | B | 231 | 13.334 | −53.258 | −30.599 | 1.00 | 54.91 | O |
| ATOM | 1 | N | VAL | B | 232 | 13.057 | −55.032 | −31.982 | 1.00 | 54.99 | N |
| ATOM | 2 | CA | VAL | B | 232 | 12.296 | −54.267 | −32.966 | 1.00 | 54.96 | C |
| ATOM | 3 | CB | VAL | B | 232 | 11.980 | −55.100 | −34.219 | 1.00 | 54.88 | C |
| ATOM | 4 | CG1 | VAL | B | 232 | 10.943 | −54.407 | −35.060 | 1.00 | 55.17 | C |
| ATOM | 5 | CG2 | VAL | B | 232 | 13.228 | −55.310 | −35.035 | 1.00 | 55.17 | C |
| ATOM | 6 | C | VAL | B | 232 | 11.006 | −53.702 | −32.365 | 1.00 | 54.91 | C |
| ATOM | 7 | O | VAL | B | 232 | 10.780 | −52.485 | −32.380 | 1.00 | 54.68 | O |
| ATOM | 1 | N | TYR | B | 233 | 10.168 | −54.583 | −31.828 | 1.00 | 54.85 | N |
| ATOM | 2 | CA | TYR | B | 233 | 8.951 | −54.152 | −31.159 | 1.00 | 54.67 | C |
| ATOM | 3 | CB | TYR | B | 233 | 8.336 | −55.333 | −30.413 | 1.00 | 54.48 | C |
| ATOM | 4 | CG | TYR | B | 233 | 7.076 | −55.026 | −29.642 | 1.00 | 54.61 | C |
| ATOM | 5 | CD1 | TYR | B | 233 | 6.701 | −55.811 | −28.562 | 1.00 | 54.70 | C |
| ATOM | 6 | CE1 | TYR | B | 233 | 5.552 | −55.544 | −27.848 | 1.00 | 54.44 | C |
| ATOM | 7 | CZ | TYR | B | 233 | 4.764 | −54.480 | −28.205 | 1.00 | 54.26 | C |
| ATOM | 8 | OH | TYR | B | 233 | 3.623 | −54.219 | −27.489 | 1.00 | 54.47 | O |
| ATOM | 9 | CE2 | TYR | B | 233 | 5.112 | −53.681 | −29.271 | 1.00 | 54.50 | C |
| ATOM | 10 | CD2 | TYR | B | 233 | 6.263 | −53.955 | −29.985 | 1.00 | 54.77 | C |
| ATOM | 11 | C | TYR | B | 233 | 9.244 | −52.964 | −30.226 | 1.00 | 54.73 | C |
| ATOM | 12 | O | TYR | B | 233 | 8.578 | −51.909 | −30.297 | 1.00 | 54.67 | O |
| ATOM | 1 | N | GLY | B | 234 | 10.257 | −53.129 | −29.375 | 1.00 | 54.80 | N |
| ATOM | 2 | CA | GLY | B | 234 | 10.703 | −52.047 | −28.502 | 1.00 | 54.70 | C |
| ATOM | 3 | C | GLY | B | 234 | 10.935 | −50.754 | −29.268 | 1.00 | 54.80 | C |
| ATOM | 4 | O | GLY | B | 234 | 10.356 | −49.698 | −28.933 | 1.00 | 54.91 | O |
| ATOM | 1 | N | ALA | B | 235 | 11.770 | −50.830 | −30.305 | 1.00 | 54.65 | N |
| ATOM | 2 | CA | ALA | B | 235 | 12.066 | −49.651 | −31.120 | 1.00 | 54.39 | C |
| ATOM | 3 | CB | ALA | B | 235 | 13.000 | −50.015 | −32.250 | 1.00 | 54.36 | C |
| ATOM | 4 | C | ALA | B | 235 | 10.792 | −49.002 | −31.667 | 1.00 | 54.45 | C |
| ATOM | 5 | O | ALA | B | 235 | 10.587 | −47.784 | −31.538 | 1.00 | 54.48 | O |
| ATOM | 1 | N | LEU | B | 236 | 9.936 | −49.824 | −32.269 | 1.00 | 54.43 | N |
| ATOM | 2 | CA | LEU | B | 236 | 8.672 | −49.355 | −32.815 | 1.00 | 54.31 | C |
| ATOM | 3 | CB | LEU | B | 236 | 7.805 | −50.532 | −33.245 | 1.00 | 54.32 | C |
| ATOM | 4 | CG | LEU | B | 236 | 6.964 | −50.421 | −34.521 | 1.00 | 54.14 | C |
| ATOM | 5 | CD1 | LEU | B | 236 | 6.530 | −49.007 | −34.824 | 1.00 | 54.03 | C |
| ATOM | 6 | CD2 | LEU | B | 236 | 5.765 | −51.350 | −34.440 | 1.00 | 53.68 | C |
| ATOM | 7 | C | LEU | B | 236 | 7.923 | −48.558 | −31.768 | 1.00 | 54.43 | C |
| ATOM | 8 | O | LEU | B | 236 | 7.626 | −47.383 | −31.973 | 1.00 | 54.51 | O |
| ATOM | 1 | N | MET | B | 237 | 7.615 | −49.192 | −30.638 | 1.00 | 54.66 | N |
| ATOM | 2 | CA | MET | B | 237 | 6.798 | −48.506 | −29.630 | 1.00 | 54.81 | C |
| ATOM | 3 | CB | MET | B | 237 | 6.388 | −49.435 | −28.488 | 1.00 | 54.86 | C |
| ATOM | 4 | CG | MET | B | 237 | 5.420 | −50.528 | −28.919 | 1.00 | 55.85 | C |
| ATOM | 5 | SD | MET | B | 237 | 3.853 | −49.943 | −29.618 | 1.00 | 57.46 | S |
| ATOM | 6 | CE | MET | B | 237 | 2.887 | −49.728 | −28.124 | 1.00 | 56.82 | C |
| ATOM | 7 | C | MET | B | 237 | 7.470 | −47.250 | −29.097 | 1.00 | 54.74 | C |
| ATOM | 8 | O | MET | B | 237 | 6.792 | −46.278 | −28.762 | 1.00 | 54.99 | O |
| ATOM | 1 | N | TRP | B | 238 | 8.796 | −47.261 | −29.028 | 1.00 | 54.61 | N |
| ATOM | 2 | CA | TRP | B | 238 | 9.517 | −46.061 | −28.619 | 1.00 | 54.49 | C |
| ATOM | 3 | CB | TRP | B | 238 | 11.004 | −46.358 | −28.620 | 1.00 | 54.42 | C |
| ATOM | 4 | CG | TRP | B | 238 | 11.685 | −45.961 | −27.380 | 1.00 | 54.35 | C |
| ATOM | 5 | CD1 | TRP | B | 238 | 11.545 | −46.533 | −26.150 | 1.00 | 54.85 | C |
| ATOM | 6 | NE1 | TRP | B | 238 | 12.352 | −45.899 | −25.236 | 1.00 | 54.83 | N |
| ATOM | 7 | CE2 | TRP | B | 238 | 13.037 | −44.903 | −25.877 | 1.00 | 54.14 | C |
| ATOM | 8 | CD2 | TRP | B | 238 | 12.639 | −44.916 | −27.231 | 1.00 | 53.92 | C |
| ATOM | 9 | CE3 | TRP | B | 238 | 13.196 | −43.991 | −28.107 | 1.00 | 53.71 | C |
| ATOM | 10 | CZ3 | TRP | B | 238 | 14.112 | −43.100 | −27.618 | 1.00 | 54.62 | C |
| ATOM | 11 | CH2 | TRP | B | 238 | 14.486 | −43.109 | −26.266 | 1.00 | 54.60 | C |
| ATOM | 12 | CZ2 | TRP | B | 238 | 13.961 | −44.004 | −25.384 | 1.00 | 54.05 | C |
| ATOM | 13 | C | TRP | B | 238 | 9.237 | −44.921 | −29.596 | 1.00 | 54.52 | C |
| ATOM | 14 | O | TRP | B | 238 | 8.748 | −43.827 | −29.230 | 1.00 | 54.67 | O |
| ATOM | 1 | N | ALA | B | 239 | 9.549 | −45.206 | −30.855 | 1.00 | 54.59 | N |
| ATOM | 2 | CA | ALA | B | 239 | 9.352 | −44.262 | −31.942 | 1.00 | 54.74 | C |
| ATOM | 3 | CB | ALA | B | 239 | 9.719 | −44.915 | −33.266 | 1.00 | 54.68 | C |
| ATOM | 4 | C | ALA | B | 239 | 7.917 | −43.746 | −31.979 | 1.00 | 54.79 | C |
| ATOM | 5 | O | ALA | B | 239 | 7.672 | −42.547 | −31.812 | 1.00 | 55.14 | O |
| ATOM | 1 | N | LEU | B | 240 | 6.975 | −44.661 | −32.203 | 1.00 | 54.72 | N |
| ATOM | 2 | CA | LEU | B | 240 | 5.561 | −44.311 | −32.240 | 1.00 | 54.66 | C |
| ATOM | 3 | CB | LEU | B | 240 | 4.693 | −45.565 | −32.320 | 1.00 | 54.59 | C |
| ATOM | 4 | CG | LEU | B | 240 | 4.544 | −46.206 | −33.699 | 1.00 | 54.30 | C |
| ATOM | 5 | CD1 | LEU | B | 240 | 3.564 | −47.363 | −33.633 | 1.00 | 54.48 | C |
| ATOM | 6 | CD2 | LEU | B | 240 | 4.092 | −45.186 | −34.731 | 1.00 | 53.92 | C |
| ATOM | 7 | C | LEU | B | 240 | 5.183 | −43.482 | −31.023 | 1.00 | 54.72 | C |
| ATOM | 8 | O | LEU | B | 240 | 4.561 | −42.429 | −31.154 | 1.00 | 54.60 | O |
| ATOM | 1 | N | GLY | B | 241 | 5.567 | −43.962 | −29.843 | 1.00 | 54.91 | N |
| ATOM | 2 | CA | GLY | B | 241 | 5.414 | −43.188 | −28.620 | 1.00 | 55.18 | C |
| ATOM | 3 | C | GLY | B | 241 | 5.758 | −41.726 | −28.845 | 1.00 | 55.50 | C |
| ATOM | 4 | O | GLY | B | 241 | 4.910 | −40.842 | −28.654 | 1.00 | 55.56 | O |
| ATOM | 1 | N | LYS | B | 242 | 6.997 | −41.464 | −29.262 | 1.00 | 55.54 | N |
| ATOM | 2 | CA | LYS | B | 242 | 7.430 | −40.079 | −29.518 | 1.00 | 55.84 | C |
| ATOM | 3 | CB | LYS | B | 242 | 8.936 | −40.037 | −29.805 | 1.00 | 55.91 | C |
| ATOM | 4 | CG | LYS | B | 242 | 9.394 | −38.929 | −30.744 | 1.00 | 56.07 | C |
| ATOM | 5 | CD | LYS | B | 242 | 9.523 | −37.591 | −30.035 | 1.00 | 56.51 | C |
| ATOM | 6 | CE | LYS | B | 242 | 10.365 | −36.620 | −30.852 | 1.00 | 56.40 | C |
| ATOM | 7 | NZ | LYS | B | 242 | 11.764 | −37.109 | −31.001 | 1.00 | 56.49 | N |
| ATOM | 8 | C | LYS | B | 242 | 6.641 | −39.370 | −30.634 | 1.00 | 56.01 | C |
| ATOM | 9 | O | LYS | B | 242 | 6.393 | −38.168 | −30.555 | 1.00 | 55.92 | O |
| ATOM | 1 | N | VAL | B | 243 | 6.236 | −40.121 | −31.656 | 1.00 | 56.28 | N |
| ATOM | 2 | CA | VAL | B | 243 | 5.597 | −39.540 | −32.849 | 1.00 | 56.96 | C |
| ATOM | 3 | CB | VAL | B | 243 | 5.682 | −40.487 | −34.074 | 1.00 | 57.04 | C |
| ATOM | 4 | CG1 | VAL | B | 243 | 5.080 | −39.818 | −35.311 | 1.00 | 56.83 | C |
| ATOM | 5 | CG2 | VAL | B | 243 | 7.125 | −40.904 | −34.333 | 1.00 | 57.52 | C |
| ATOM | 6 | C | VAL | B | 243 | 4.137 | −39.111 | −32.678 | 1.00 | 57.22 | C |
| ATOM | 7 | O | VAL | B | 243 | 3.826 | −37.939 | −32.860 | 1.00 | 57.33 | O |
| ATOM | 1 | N | VAL | B | 244 | 3.244 | −40.051 | −32.355 | 1.00 | 57.66 | N |
| ATOM | 2 | CA | VAL | B | 244 | 1.807 | −39.748 | −32.281 | 1.00 | 58.09 | C |
| ATOM | 3 | CB | VAL | B | 244 | 0.922 | −41.016 | −32.298 | 1.00 | 57.99 | C |
| ATOM | 4 | CG1 | VAL | B | 244 | 1.602 | −42.136 | −33.060 | 1.00 | 58.05 | C |
| ATOM | 5 | CG2 | VAL | B | 244 | 0.598 | −41.459 | −30.895 | 1.00 | 57.87 | C |
| ATOM | 6 | C | VAL | B | 244 | 1.443 | −38.882 | −31.071 | 1.00 | 58.63 | C |
| ATOM | 7 | O | VAL | B | 244 | 0.437 | −38.174 | −31.092 | 1.00 | 58.67 | O |
| ATOM | 1 | N | GLY | B | 245 | 2.253 | −38.957 | −30.017 | 1.00 | 59.32 | N |
| ATOM | 2 | CA | GLY | B | 245 | 2.209 | −37.975 | −28.923 | 1.00 | 60.12 | C |
| ATOM | 3 | C | GLY | B | 245 | 1.073 | −38.053 | −27.916 | 1.00 | 60.64 | C |
| ATOM | 4 | O | GLY | B | 245 | 1.096 | −37.358 | −26.896 | 1.00 | 60.52 | O |
| ATOM | 1 | N | THR | B | 246 | 0.078 | −38.891 | −28.202 | 1.00 | 61.23 | N |
| ATOM | 2 | CA | THR | B | 246 | −1.045 | −39.111 | −27.289 | 1.00 | 61.72 | C |
| ATOM | 3 | CB | THR | B | 246 | −2.213 | −39.822 | −28.006 | 1.00 | 61.77 | C |
| ATOM | 4 | OG1 | THR | B | 246 | −3.278 | −40.049 | −27.076 | 1.00 | 62.38 | O |
| ATOM | 5 | CG2 | THR | B | 246 | −1.764 | −41.160 | −28.576 | 1.00 | 61.63 | C |
| ATOM | 6 | C | THR | B | 246 | −0.609 | −39.947 | −26.080 | 1.00 | 61.92 | C |
| ATOM | 7 | O | THR | B | 246 | 0.221 | −40.838 | −26.217 | 1.00 | 61.87 | O |
| ATOM | 1 | N | PRO | B | 247 | −1.152 | −39.646 | −24.885 | 1.00 | 62.13 | N |
| ATOM | 2 | CA | PRO | B | 247 | −0.824 | −40.447 | −23.703 | 1.00 | 62.22 | C |
| ATOM | 3 | CB | PRO | B | 247 | −1.435 | −39.644 | −22.545 | 1.00 | 62.11 | C |
| ATOM | 4 | CG | PRO | B | 247 | −1.723 | −38.284 | −23.112 | 1.00 | 62.24 | C |
| ATOM | 5 | CD | PRO | B | 247 | −2.056 | −38.535 | −24.550 | 1.00 | 62.13 | C |
| ATOM | 6 | C | PRO | B | 247 | −1.422 | −41.857 | −23.748 | 1.00 | 62.38 | C |
| ATOM | 7 | O | PRO | B | 247 | −1.018 | −42.714 | −22.962 | 1.00 | 62.55 | O |
| ATOM | 1 | N | GLU | B | 248 | −2.369 | −42.095 | −24.655 | 1.00 | 62.37 | N |
| ATOM | 2 | CA | GLU | B | 248 | −2.954 | −43.427 | −24.818 | 1.00 | 62.39 | C |
| ATOM | 3 | CB | GLU | B | 248 | −4.428 | −43.337 | −25.218 | 1.00 | 62.54 | C |
| ATOM | 4 | CG | GLU | B | 248 | −5.252 | −42.415 | −24.341 | 1.00 | 63.55 | C |
| ATOM | 5 | CD | GLU | B | 248 | −5.301 | −41.000 | −24.881 | 1.00 | 64.86 | C |
| ATOM | 6 | OE1 | GLU | B | 248 | −5.033 | −40.048 | −24.111 | 1.00 | 65.06 | O |
| ATOM | 7 | OE2 | GLU | B | 248 | −5.603 | −40.844 | −26.085 | 1.00 | 65.69 | O |
| ATOM | 8 | C | GLU | B | 248 | −2.187 | −44.235 | −25.853 | 1.00 | 62.14 | C |
| ATOM | 9 | O | GLU | B | 248 | −2.024 | −43.801 | −26.991 | 1.00 | 62.25 | O |
| ATOM | 1 | N | VAL | B | 249 | −1.734 | −45.419 | −25.454 | 1.00 | 61.87 | N |
| ATOM | 2 | CA | VAL | B | 249 | −0.874 | −46.254 | −26.293 | 1.00 | 61.58 | C |
| ATOM | 3 | CB | VAL | B | 249 | 0.014 | −47.184 | −25.424 | 1.00 | 61.51 | C |
| ATOM | 4 | CG1 | VAL | B | 249 | −0.721 | −47.608 | −24.163 | 1.00 | 61.83 | C |
| ATOM | 5 | CG2 | VAL | B | 249 | 0.465 | −48.396 | −26.209 | 1.00 | 61.54 | C |
| ATOM | 6 | C | VAL | B | 249 | −1.634 | −47.072 | −27.345 | 1.00 | 61.44 | C |
| ATOM | 7 | O | VAL | B | 249 | −2.670 | −47.671 | −27.060 | 1.00 | 61.45 | O |
| ATOM | 1 | N | LEU | B | 250 | −1.108 | −47.082 | −28.565 | 1.00 | 61.36 | N |
| ATOM | 2 | CA | LEU | B | 250 | −1.689 | −47.851 | −29.659 | 1.00 | 61.27 | C |
| ATOM | 3 | CB | LEU | B | 250 | −1.162 | −47.347 | −31.002 | 1.00 | 61.02 | C |
| ATOM | 4 | CG | LEU | B | 250 | −2.082 | −46.481 | −31.862 | 1.00 | 60.92 | C |
| ATOM | 5 | CD1 | LEU | B | 250 | −2.866 | −45.482 | −31.035 | 1.00 | 61.76 | C |
| ATOM | 6 | CD2 | LEU | B | 250 | −1.277 | −45.765 | −32.921 | 1.00 | 61.02 | C |
| ATOM | 7 | C | LEU | B | 250 | −1.388 | −49.335 | −29.522 | 1.00 | 61.49 | C |
| ATOM | 8 | O | LEU | B | 250 | −0.289 | −49.725 | −29.129 | 1.00 | 61.64 | O |
| ATOM | 1 | N | ARG | B | 251 | −2.371 | −50.161 | −29.858 | 1.00 | 61.69 | N |
| ATOM | 2 | CA | ARG | B | 251 | −2.193 | −51.606 | −29.865 | 1.00 | 61.74 | C |
| ATOM | 3 | CB | ARG | B | 251 | −3.560 | −52.294 | −29.827 | 1.00 | 61.77 | C |
| ATOM | 4 | CG | ARG | B | 251 | −3.556 | −53.780 | −30.178 | 1.00 | 61.82 | C |
| ATOM | 5 | CD | ARG | B | 251 | −3.114 | −54.629 | −28.998 | 1.00 | 61.89 | C |
| ATOM | 6 | NE | ARG | B | 251 | −3.929 | −55.833 | −28.873 | 1.00 | 61.71 | N |
| ATOM | 7 | CZ | ARG | B | 251 | −3.557 | −57.040 | −29.285 | 1.00 | 61.63 | C |
| ATOM | 8 | NH1 | ARG | B | 251 | −2.374 | −57.216 | −29.853 | 1.00 | 61.74 | N |
| ATOM | 9 | NH2 | ARG | B | 251 | −4.375 | −58.072 | −29.132 | 1.00 | 61.58 | N |
| ATOM | 10 | C | ARG | B | 251 | −1.446 | −52.033 | −31.121 | 1.00 | 61.79 | C |
| ATOM | 11 | O | ARG | B | 251 | −1.820 | −51.641 | −32.227 | 1.00 | 61.72 | O |
| ATOM | 1 | N | VAL | B | 252 | −0.390 | −52.825 | −30.954 | 1.00 | 61.91 | N |
| ATOM | 2 | CA | VAL | B | 252 | 0.235 | −53.487 | −32.105 | 1.00 | 62.11 | C |
| ATOM | 3 | CB | VAL | B | 252 | 1.776 | −53.317 | −32.142 | 1.00 | 62.15 | C |
| ATOM | 4 | CG1 | VAL | B | 252 | 2.143 | −51.846 | −32.199 | 1.00 | 62.31 | C |
| ATOM | 5 | CG2 | VAL | B | 252 | 2.415 | −53.976 | −30.934 | 1.00 | 62.44 | C |
| ATOM | 6 | C | VAL | B | 252 | −0.125 | −54.972 | −32.144 | 1.00 | 61.92 | C |
| ATOM | 7 | O | VAL | B | 252 | −0.175 | −55.633 | −31.107 | 1.00 | 61.98 | O |
| ATOM | 1 | N | TYR | B | 253 | −0.394 | −55.482 | −33.342 | 1.00 | 61.76 | N |
| ATOM | 2 | CA | TYR | B | 253 | −0.691 | −56.898 | −33.532 | 1.00 | 61.60 | C |
| ATOM | 3 | CB | TYR | B | 253 | −1.914 | −57.072 | −34.434 | 1.00 | 61.60 | C |
| ATOM | 4 | CG | TYR | B | 253 | −3.179 | −56.478 | −33.847 | 1.00 | 62.19 | C |
| ATOM | 5 | CD1 | TYR | B | 253 | −3.706 | −55.293 | −34.334 | 1.00 | 62.55 | C |
| ATOM | 6 | CE1 | TYR | B | 253 | −4.859 | −54.749 | −33.792 | 1.00 | 62.44 | C |
| ATOM | 7 | CZ | TYR | B | 253 | −5.493 | −55.384 | −32.751 | 1.00 | 61.97 | C |
| ATOM | 8 | OH | TYR | B | 253 | −6.639 | −54.844 | −32.210 | 1.00 | 62.06 | O |
| ATOM | 9 | CE2 | TYR | B | 253 | −4.990 | −56.557 | −32.251 | 1.00 | 62.23 | C |
| ATOM | 10 | CD2 | TYR | B | 253 | −3.841 | −57.098 | −32.795 | 1.00 | 62.57 | C |
| ATOM | 11 | C | TYR | B | 253 | 0.524 | −57.588 | −34.134 | 1.00 | 61.37 | C |
| ATOM | 12 | O | TYR | B | 253 | 0.937 | −57.252 | −35.243 | 1.00 | 61.52 | O |
| ATOM | 1 | N | ILE | B | 254 | 1.100 | −58.539 | −33.399 | 1.00 | 60.85 | N |
| ATOM | 2 | CA | ILE | B | 254 | 2.348 | −59.177 | −33.815 | 1.00 | 60.38 | C |
| ATOM | 3 | CB | ILE | B | 254 | 3.255 | −59.455 | −32.608 | 1.00 | 60.34 | C |
| ATOM | 4 | CG1 | ILE | B | 254 | 3.199 | −58.278 | −31.632 | 1.00 | 60.50 | C |
| ATOM | 5 | CD1 | ILE | B | 254 | 4.262 | −58.319 | −30.554 | 1.00 | 60.93 | C |
| ATOM | 6 | CG2 | ILE | B | 254 | 4.679 | −59.735 | −33.062 | 1.00 | 59.77 | C |
| ATOM | 7 | C | ILE | B | 254 | 2.115 | −60.486 | −34.559 | 1.00 | 60.21 | C |
| ATOM | 8 | O | ILE | B | 254 | 1.542 | −61.423 | −34.016 | 1.00 | 60.30 | O |
| ATOM | 1 | N | GLY | B | 255 | 2.569 | −60.558 | −35.804 | 1.00 | 59.92 | N |
| ATOM | 2 | CA | GLY | B | 255 | 2.410 | −61.789 | −36.573 | 1.00 | 59.62 | C |
| ATOM | 3 | C | GLY | B | 255 | 3.016 | −61.725 | −37.960 | 1.00 | 59.23 | C |
| ATOM | 4 | O | GLY | B | 255 | 3.708 | −60.766 | −38.298 | 1.00 | 59.30 | O |
| ATOM | 1 | N | SER | B | 256 | 2.765 | −62.759 | −38.757 | 1.00 | 58.78 | N |
| ATOM | 2 | CA | SER | B | 256 | 3.158 | −62.757 | −40.160 | 1.00 | 58.69 | C |
| ATOM | 3 | CB | SER | B | 256 | 4.419 | −63.593 | −40.397 | 1.00 | 58.72 | C |
| ATOM | 4 | OG | SER | B | 256 | 4.402 | −64.776 | −39.623 | 1.00 | 59.59 | O |
| ATOM | 5 | C | SER | B | 256 | 2.000 | −63.204 | −41.054 | 1.00 | 58.34 | C |
| ATOM | 6 | O | SER | B | 256 | 1.769 | −64.392 | −41.277 | 1.00 | 58.22 | O |
| ATOM | 1 | N | PHE | B | 257 | 1.293 | −62.212 | −41.578 | 1.00 | 58.01 | N |
| ATOM | 2 | CA | PHE | B | 257 | 0.041 | −62.410 | −42.277 | 1.00 | 57.54 | C |
| ATOM | 3 | CB | PHE | B | 257 | −0.756 | −61.107 | −42.223 | 1.00 | 57.11 | C |
| ATOM | 4 | CG | PHE | B | 257 | −1.001 | −60.627 | −40.821 | 1.00 | 56.81 | C |
| ATOM | 5 | CD1 | PHE | B | 257 | −2.247 | −60.763 | −40.232 | 1.00 | 56.40 | C |
| ATOM | 6 | CE1 | PHE | B | 257 | −2.469 | −60.338 | −38.933 | 1.00 | 55.40 | C |
| ATOM | 7 | CZ | PHE | B | 257 | −1.441 | −59.781 | −38.204 | 1.00 | 55.70 | C |
| ATOM | 8 | CE2 | PHE | B | 257 | −0.193 | −59.650 | −38.764 | 1.00 | 55.89 | C |
| ATOM | 9 | CD2 | PHE | B | 257 | 0.027 | −60.077 | −40.070 | 1.00 | 56.91 | C |
| ATOM | 10 | C | PHE | B | 257 | 0.200 | −62.948 | −43.706 | 1.00 | 57.57 | C |
| ATOM | 11 | O | PHE | B | 257 | 0.067 | −62.216 | −44.690 | 1.00 | 57.54 | O |
| ATOM | 1 | N | TRP | B | 258 | 0.492 | −64.243 | −43.793 | 1.00 | 57.58 | N |
| ATOM | 2 | CA | TRP | B | 258 | 0.511 | −64.966 | −45.062 | 1.00 | 57.65 | C |
| ATOM | 3 | CB | TRP | B | 258 | 1.642 | −64.464 | −45.966 | 1.00 | 57.76 | C |
| ATOM | 4 | CG | TRP | B | 258 | 3.022 | −64.843 | −45.544 | 1.00 | 57.87 | C |
| ATOM | 5 | CD1 | TRP | B | 258 | 3.423 | −65.277 | −44.311 | 1.00 | 58.43 | C |
| ATOM | 6 | NE1 | TRP | B | 258 | 4.777 | −65.520 | −44.311 | 1.00 | 58.60 | N |
| ATOM | 7 | CE2 | TRP | B | 258 | 5.278 | −65.229 | −45.554 | 1.00 | 58.63 | C |
| ATOM | 8 | CD2 | TRP | B | 258 | 4.201 | −64.791 | −46.353 | 1.00 | 58.19 | C |
| ATOM | 9 | CE3 | TRP | B | 258 | 4.450 | −64.430 | −47.682 | 1.00 | 58.21 | C |
| ATOM | 10 | CZ3 | TRP | B | 258 | 5.747 | −64.513 | −48.163 | 1.00 | 58.20 | C |
| ATOM | 11 | CH2 | TRP | B | 258 | 6.797 | −64.949 | −47.344 | 1.00 | 58.12 | C |
| ATOM | 12 | CZ2 | TRP | B | 258 | 6.584 | −65.313 | −46.041 | 1.00 | 58.37 | C |
| ATOM | 13 | C | TRP | B | 258 | 0.572 | −66.479 | −44.825 | 1.00 | 57.67 | C |
| ATOM | 14 | O | TRP | B | 258 | 1.064 | −66.937 | −43.790 | 1.00 | 57.71 | O |
| ATOM | 1 | N | SER | B | 259 | 0.057 | −67.254 | −45.776 | 1.00 | 57.66 | N |
| ATOM | 2 | CA | SER | B | 259 | −0.078 | −68.706 | −45.598 | 1.00 | 57.55 | C |
| ATOM | 3 | CB | SER | B | 259 | −1.160 | −69.255 | −46.527 | 1.00 | 57.60 | C |
| ATOM | 4 | OG | SER | B | 259 | −2.416 | −68.667 | −46.232 | 1.00 | 58.07 | O |
| ATOM | 5 | C | SER | B | 259 | 1.226 | −69.478 | −45.802 | 1.00 | 57.36 | C |
| ATOM | 6 | O | SER | B | 259 | 1.217 | −70.686 | −46.047 | 1.00 | 57.37 | O |
| ATOM | 1 | N | GLN | B | 260 | 2.344 | −68.775 | −45.689 | 1.00 | 57.11 | N |
| ATOM | 2 | CA | GLN | B | 260 | 3.650 | −69.384 | −45.862 | 1.00 | 56.90 | C |
| ATOM | 3 | CB | GLN | B | 260 | 4.659 | −68.302 | −46.240 | 1.00 | 57.05 | C |
| ATOM | 4 | CG | GLN | B | 260 | 5.638 | −68.687 | −47.342 | 1.00 | 58.16 | C |
| ATOM | 5 | CD | GLN | B | 260 | 4.957 | −68.952 | −48.679 | 1.00 | 59.01 | C |
| ATOM | 6 | OE1 | GLN | B | 260 | 3.889 | −68.404 | −48.970 | 1.00 | 59.19 | O |
| ATOM | 7 | NE2 | GLN | B | 260 | 5.578 | −69.798 | −49.499 | 1.00 | 58.96 | N |
| ATOM | 8 | C | GLN | B | 260 | 4.059 | −70.055 | −44.556 | 1.00 | 56.43 | C |
| ATOM | 9 | O | GLN | B | 260 | 3.621 | −69.640 | −43.487 | 1.00 | 56.50 | O |
| ATOM | 1 | N | PRO | B | 261 | 4.877 | −71.113 | −44.634 | 1.00 | 56.02 | N |
| ATOM | 2 | CA | PRO | B | 261 | 5.441 | −71.724 | −43.431 | 1.00 | 55.81 | C |
| ATOM | 3 | CB | PRO | B | 261 | 6.390 | −72.785 | −43.990 | 1.00 | 55.75 | C |
| ATOM | 4 | CG | PRO | B | 261 | 5.810 | −73.132 | −45.313 | 1.00 | 55.87 | C |
| ATOM | 5 | CD | PRO | B | 261 | 5.277 | −71.832 | −45.853 | 1.00 | 56.03 | C |
| ATOM | 6 | C | PRO | B | 261 | 6.208 | −70.729 | −42.564 | 1.00 | 55.59 | C |
| ATOM | 7 | O | PRO | B | 261 | 6.579 | −69.652 | −43.024 | 1.00 | 55.74 | O |
| ATOM | 1 | N | LEU | B | 262 | 6.443 | −71.092 | −41.313 | 1.00 | 55.30 | N |
| ATOM | 2 | CA | LEU | B | 262 | 7.037 | −70.173 | −40.365 | 1.00 | 55.12 | C |
| ATOM | 3 | CB | LEU | B | 262 | 6.474 | −70.457 | −38.978 | 1.00 | 55.09 | C |
| ATOM | 4 | CG | LEU | B | 262 | 5.736 | −69.356 | −38.216 | 1.00 | 54.85 | C |
| ATOM | 5 | CD1 | LEU | B | 262 | 5.003 | −68.395 | −39.142 | 1.00 | 54.87 | C |
| ATOM | 6 | CD2 | LEU | B | 262 | 4.758 | −70.010 | −37.276 | 1.00 | 54.29 | C |
| ATOM | 7 | C | LEU | B | 262 | 8.534 | −70.386 | −40.370 | 1.00 | 55.14 | C |
| ATOM | 8 | O | LEU | B | 262 | 8.992 | −71.518 | −40.268 | 1.00 | 55.24 | O |
| ATOM | 1 | N | LEU | B | 263 | 9.302 | −69.309 | −40.504 | 1.00 | 55.24 | N |
| ATOM | 2 | CA | LEU | B | 263 | 10.757 | −69.433 | −40.527 | 1.00 | 55.20 | C |
| ATOM | 3 | CB | LEU | B | 263 | 11.424 | −68.110 | −40.916 | 1.00 | 55.14 | C |
| ATOM | 4 | CG | LEU | B | 263 | 12.794 | −68.176 | −41.612 | 1.00 | 55.03 | C |
| ATOM | 5 | CD1 | LEU | B | 263 | 13.368 | −66.781 | −41.784 | 1.00 | 54.72 | C |
| ATOM | 6 | CD2 | LEU | B | 263 | 13.794 | −69.056 | −40.873 | 1.00 | 55.07 | C |
| ATOM | 7 | C | LEU | B | 263 | 11.234 | −69.875 | −39.154 | 1.00 | 55.30 | C |
| ATOM | 8 | O | LEU | B | 263 | 11.621 | −71.025 | −38.962 | 1.00 | 55.01 | O |
| ATOM | 1 | N | VAL | B | 264 | 11.202 | −68.949 | −38.203 | 1.00 | 55.62 | N |
| ATOM | 2 | CA | VAL | B | 264 | 11.550 | −69.260 | −36.824 | 1.00 | 55.97 | C |
| ATOM | 3 | CB | VAL | B | 264 | 12.484 | −68.194 | −36.214 | 1.00 | 55.83 | C |
| ATOM | 4 | CG1 | VAL | B | 264 | 12.011 | −66.801 | −36.566 | 1.00 | 55.89 | C |
| ATOM | 5 | CG2 | VAL | B | 264 | 12.602 | −68.376 | −34.708 | 1.00 | 56.05 | C |
| ATOM | 6 | C | VAL | B | 264 | 10.272 | −69.448 | −36.007 | 1.00 | 56.23 | C |
| ATOM | 7 | O | VAL | B | 264 | 9.618 | −68.484 | −35.632 | 1.00 | 56.29 | O |
| ATOM | 1 | N | PRO | B | 265 | 9.926 | −70.711 | −35.722 | 1.00 | 56.68 | N |
| ATOM | 2 | CA | PRO | B | 265 | 8.590 | −71.109 | −35.296 | 1.00 | 57.10 | C |
| ATOM | 3 | CB | PRO | B | 265 | 8.583 | −72.607 | −35.601 | 1.00 | 57.09 | C |
| ATOM | 4 | CG | PRO | B | 265 | 9.988 | −73.022 | −35.346 | 1.00 | 56.77 | C |
| ATOM | 5 | CD | PRO | B | 265 | 10.843 | −71.863 | −35.792 | 1.00 | 56.60 | C |
| ATOM | 6 | C | PRO | B | 265 | 8.272 | −70.900 | −33.816 | 1.00 | 57.60 | C |
| ATOM | 7 | O | PRO | B | 265 | 7.098 | −70.934 | −33.434 | 1.00 | 57.55 | O |
| ATOM | 1 | N | ASP | B | 266 | 9.290 | −70.686 | −32.987 | 1.00 | 58.11 | N |
| ATOM | 2 | CA | ASP | B | 266 | 9.079 | −70.770 | −31.542 | 1.00 | 58.60 | C |
| ATOM | 3 | CB | ASP | B | 266 | 10.389 | −71.053 | −30.775 | 1.00 | 58.96 | C |
| ATOM | 4 | CG | ASP | B | 266 | 11.533 | −70.141 | −31.181 | 1.00 | 59.26 | C |
| ATOM | 5 | OD1 | ASP | B | 266 | 11.436 | −68.920 | −30.940 | 1.00 | 60.12 | O |
| ATOM | 6 | OD2 | ASP | B | 266 | 12.545 | −70.659 | −31.706 | 1.00 | 59.63 | O |
| ATOM | 7 | C | ASP | B | 266 | 8.295 | −69.617 | −30.932 | 1.00 | 58.63 | C |
| ATOM | 8 | O | ASP | B | 266 | 8.522 | −69.238 | −29.791 | 1.00 | 58.80 | O |
| ATOM | 1 | N | ASN | B | 267 | 7.349 | −69.085 | −31.693 | 1.00 | 58.79 | N |
| ATOM | 2 | CA | ASN | B | 267 | 6.502 | −68.002 | −31.220 | 1.00 | 58.87 | C |
| ATOM | 3 | CB | ASN | B | 267 | 7.161 | −66.657 | −31.514 | 1.00 | 58.66 | C |
| ATOM | 4 | CG | ASN | B | 267 | 7.883 | −66.102 | −30.322 | 1.00 | 58.19 | C |
| ATOM | 5 | OD1 | ASN | B | 267 | 7.410 | −66.229 | −29.197 | 1.00 | 58.89 | O |
| ATOM | 6 | ND2 | ASN | B | 267 | 9.033 | −65.483 | −30.552 | 1.00 | 57.38 | N |
| ATOM | 7 | C | ASN | B | 267 | 5.113 | −68.040 | −31.846 | 1.00 | 59.27 | C |
| ATOM | 8 | O | ASN | B | 267 | 4.383 | −67.045 | −31.818 | 1.00 | 59.19 | O |
| ATOM | 1 | N | ARG | B | 268 | 4.737 | −69.187 | −32.406 | 1.00 | 59.60 | N |
| ATOM | 2 | CA | ARG | B | 268 | 3.543 | −69.204 | −33.234 | 1.00 | 59.99 | C |
| ATOM | 3 | CB | ARG | B | 268 | 3.514 | −70.400 | −34.200 | 1.00 | 60.07 | C |
| ATOM | 4 | CG | ARG | B | 268 | 2.838 | −71.672 | −33.768 | 1.00 | 60.90 | C |
| ATOM | 5 | CD | ARG | B | 268 | 2.359 | −72.379 | −35.039 | 1.00 | 63.18 | C |
| ATOM | 6 | NE | ARG | B | 268 | 2.404 | −73.840 | −34.985 | 1.00 | 65.28 | N |
| ATOM | 7 | CZ | ARG | B | 268 | 3.504 | −74.570 | −35.179 | 1.00 | 66.78 | C |
| ATOM | 8 | NH1 | ARG | B | 268 | 4.670 | −73.975 | −35.423 | 1.00 | 66.33 | N |
| ATOM | 9 | NH2 | ARG | B | 268 | 3.443 | −75.902 | −35.119 | 1.00 | 67.27 | N |
| ATOM | 10 | C | ARG | B | 268 | 2.259 | −69.016 | −32.443 | 1.00 | 60.01 | C |
| ATOM | 11 | O | ARG | B | 268 | 1.344 | −68.351 | −32.913 | 1.00 | 60.20 | O |
| ATOM | 1 | N | ARG | B | 269 | 2.213 | −69.550 | −31.229 | 1.00 | 60.18 | N |
| ATOM | 2 | CA | ARG | B | 269 | 1.086 | −69.298 | −30.335 | 1.00 | 60.31 | C |
| ATOM | 3 | CB | ARG | B | 269 | 1.421 | −69.759 | −28.916 | 1.00 | 60.47 | C |
| ATOM | 4 | CG | ARG | B | 269 | 0.627 | −70.966 | −28.439 | 1.00 | 61.18 | C |
| ATOM | 5 | CD | ARG | B | 269 | −0.752 | −70.541 | −27.930 | 1.00 | 62.47 | C |
| ATOM | 6 | NE | ARG | B | 269 | −1.734 | −70.391 | −29.005 | 1.00 | 62.90 | N |
| ATOM | 7 | CZ | ARG | B | 269 | −2.957 | −70.919 | −28.983 | 1.00 | 62.99 | C |
| ATOM | 8 | NH1 | ARG | B | 269 | −3.366 | −71.619 | −27.931 | 1.00 | 62.79 | N |
| ATOM | 9 | NH2 | ARG | B | 269 | −3.781 | −70.729 | −30.008 | 1.00 | 62.86 | N |
| ATOM | 10 | C | ARG | B | 269 | 0.716 | −67.818 | −30.329 | 1.00 | 60.15 | C |
| ATOM | 11 | O | ARG | B | 269 | −0.443 | −67.449 | −30.540 | 1.00 | 60.18 | O |
| ATOM | 1 | N | LEU | B | 270 | 1.719 | −66.978 | −30.094 | 1.00 | 60.00 | N |
| ATOM | 2 | CA | LEU | B | 270 | 1.530 | −65.539 | −30.035 | 1.00 | 59.87 | C |
| ATOM | 3 | CB | LEU | B | 270 | 2.863 | −64.857 | −29.733 | 1.00 | 59.80 | C |
| ATOM | 4 | CG | LEU | B | 270 | 2.898 | −63.460 | −29.106 | 1.00 | 59.73 | C |
| ATOM | 5 | CD1 | LEU | B | 270 | 3.052 | −62.370 | −30.149 | 1.00 | 59.80 | C |
| ATOM | 6 | CD2 | LEU | B | 270 | 1.683 | −63.214 | −28.229 | 1.00 | 59.78 | C |
| ATOM | 7 | C | LEU | B | 270 | 0.957 | −65.031 | −31.346 | 1.00 | 59.92 | C |
| ATOM | 8 | O | LEU | B | 270 | −0.016 | −64.287 | −31.358 | 1.00 | 60.09 | O |
| ATOM | 1 | N | PHE | B | 271 | 1.552 | −65.455 | −32.454 | 1.00 | 60.22 | N |
| ATOM | 2 | CA | PHE | B | 271 | 1.123 | −65.006 | −33.774 | 1.00 | 60.39 | C |
| ATOM | 3 | CB | PHE | B | 271 | 2.020 | −65.581 | −34.870 | 1.00 | 60.47 | C |
| ATOM | 4 | CG | PHE | B | 271 | 3.471 | −65.224 | −34.717 | 1.00 | 60.39 | C |
| ATOM | 5 | CD1 | PHE | B | 271 | 3.847 | −64.051 | −34.072 | 1.00 | 60.01 | C |
| ATOM | 6 | CE1 | PHE | B | 271 | 5.181 | −63.716 | −33.930 | 1.00 | 59.82 | C |
| ATOM | 7 | CZ | PHE | B | 271 | 6.159 | −64.546 | −34.451 | 1.00 | 60.43 | C |
| ATOM | 8 | CE2 | PHE | B | 271 | 5.798 | −65.718 | −35.105 | 1.00 | 60.27 | C |
| ATOM | 9 | CD2 | PHE | B | 271 | 4.458 | −66.049 | −35.237 | 1.00 | 60.08 | C |
| ATOM | 10 | C | PHE | B | 271 | −0.326 | −65.375 | −34.044 | 1.00 | 60.56 | C |
| ATOM | 11 | O | PHE | B | 271 | −1.129 | −64.523 | −34.451 | 1.00 | 60.91 | O |
| ATOM | 1 | N | GLU | B | 272 | −0.672 | −66.638 | −33.819 | 1.00 | 60.53 | N |
| ATOM | 2 | CA | GLU | B | 272 | −2.057 | −67.032 | −34.002 | 1.00 | 60.88 | C |
| ATOM | 3 | CB | GLU | B | 272 | −2.280 | −68.554 | −33.896 | 1.00 | 60.68 | C |
| ATOM | 4 | CG | GLU | B | 272 | −1.359 | −69.330 | −32.967 | 1.00 | 61.39 | C |
| ATOM | 5 | CD | GLU | B | 272 | −1.554 | −70.852 | −33.082 | 1.00 | 62.06 | C |
| ATOM | 6 | OE1 | GLU | B | 272 | −1.025 | −71.608 | −32.228 | 1.00 | 63.50 | O |
| ATOM | 7 | OE2 | GLU | B | 272 | −2.245 | −71.301 | −34.030 | 1.00 | 64.06 | O |
| ATOM | 8 | C | GLU | B | 272 | −2.972 | −66.221 | −33.079 | 1.00 | 60.54 | C |
| ATOM | 9 | O | GLU | B | 272 | −4.000 | −65.707 | −33.518 | 1.00 | 60.78 | O |
| ATOM | 1 | N | LEU | B | 273 | −2.582 | −66.061 | −31.819 | 1.00 | 60.28 | N |
| ATOM | 2 | CA | LEU | B | 273 | −3.385 | −65.255 | −30.904 | 1.00 | 60.15 | C |
| ATOM | 3 | CB | LEU | B | 273 | −2.765 | −65.249 | −29.505 | 1.00 | 60.05 | C |
| ATOM | 4 | CG | LEU | B | 273 | −3.567 | −66.013 | −28.454 | 1.00 | 59.60 | C |
| ATOM | 5 | CD1 | LEU | B | 273 | −5.001 | −65.525 | −28.500 | 1.00 | 60.64 | C |
| ATOM | 6 | CD2 | LEU | B | 273 | −3.523 | −67.492 | −28.688 | 1.00 | 59.04 | C |
| ATOM | 7 | C | LEU | B | 273 | −3.635 | −63.819 | −31.402 | 1.00 | 60.30 | C |
| ATOM | 8 | O | LEU | B | 273 | −4.785 | −63.350 | −31.454 | 1.00 | 60.39 | O |
| ATOM | 1 | N | GLU | B | 274 | −2.559 | −63.128 | −31.769 | 1.00 | 60.35 | N |
| ATOM | 2 | CA | GLU | B | 274 | −2.662 | −61.747 | −32.227 | 1.00 | 60.63 | C |
| ATOM | 3 | CB | GLU | B | 274 | −1.274 | −61.173 | −32.505 | 1.00 | 60.61 | C |
| ATOM | 4 | CG | GLU | B | 274 | −0.341 | −61.133 | −31.294 | 1.00 | 61.23 | C |
| ATOM | 5 | CD | GLU | B | 274 | −0.625 | −59.970 | −30.352 | 1.00 | 62.07 | C |
| ATOM | 6 | OE1 | GLU | B | 274 | 0.025 | −58.910 | −30.507 | 1.00 | 62.22 | O |
| ATOM | 7 | OE2 | GLU | B | 274 | −1.496 | −60.115 | −29.462 | 1.00 | 62.05 | O |
| ATOM | 8 | C | GLU | B | 274 | −3.525 | −61.672 | −33.481 | 1.00 | 60.74 | C |
| ATOM | 9 | O | GLU | B | 274 | −4.423 | −60.821 | −33.603 | 1.00 | 60.90 | O |
| ATOM | 1 | N | GLU | B | 275 | −3.252 | −62.578 | −34.410 | 1.00 | 60.89 | N |
| ATOM | 2 | CA | GLU | B | 275 | −4.061 | −62.697 | −35.607 | 1.00 | 61.09 | C |
| ATOM | 3 | CB | GLU | B | 275 | −3.606 | −63.919 | −36.392 | 1.00 | 61.28 | C |
| ATOM | 4 | CG | GLU | B | 275 | −4.061 | −63.971 | −37.832 | 1.00 | 62.13 | C |
| ATOM | 5 | CD | GLU | B | 275 | −3.647 | −65.272 | −38.492 | 1.00 | 63.38 | C |
| ATOM | 6 | OE1 | GLU | B | 275 | −4.438 | −65.821 | −39.296 | 1.00 | 63.96 | O |
| ATOM | 7 | OE2 | GLU | B | 275 | −2.533 | −65.757 | −38.185 | 1.00 | 63.30 | O |
| ATOM | 8 | C | GLU | B | 275 | −5.537 | −62.822 | −35.225 | 1.00 | 61.04 | C |
| ATOM | 9 | O | GLU | B | 275 | −6.396 | −62.138 | −35.781 | 1.00 | 60.86 | O |
| ATOM | 1 | N | GLN | B | 276 | −5.823 | −63.688 | −34.260 | 1.00 | 61.18 | N |
| ATOM | 2 | CA | GLN | B | 276 | −7.189 | −63.863 | −33.773 | 1.00 | 61.56 | C |
| ATOM | 3 | CB | GLN | B | 276 | −7.249 | −64.934 | −32.676 | 1.00 | 61.86 | C |
| ATOM | 4 | CG | GLN | B | 276 | −7.328 | −66.357 | −33.202 | 1.00 | 63.52 | C |
| ATOM | 5 | CD | GLN | B | 276 | −8.595 | −66.593 | −34.008 | 1.00 | 66.43 | C |
| ATOM | 6 | OE1 | GLN | B | 276 | −9.697 | −66.659 | −33.452 | 1.00 | 67.98 | O |
| ATOM | 7 | NE2 | GLN | B | 276 | −8.447 | −66.718 | −35.326 | 1.00 | 67.10 | N |
| ATOM | 8 | C | GLN | B | 276 | −7.782 | −62.550 | −33.265 | 1.00 | 61.36 | C |
| ATOM | 9 | O | GLN | B | 276 | −8.914 | −62.196 | −33.614 | 1.00 | 61.33 | O |
| ATOM | 1 | N | ASP | B | 277 | −7.023 | −61.827 | −32.446 | 1.00 | 61.08 | N |
| ATOM | 2 | CA | ASP | B | 277 | −7.530 | −60.572 | −31.896 | 1.00 | 61.12 | C |
| ATOM | 3 | CB | ASP | B | 277 | −6.544 | −59.973 | −30.890 | 1.00 | 61.25 | C |
| ATOM | 4 | CG | ASP | B | 277 | −6.489 | −60.757 | −29.576 | 1.00 | 62.18 | C |
| ATOM | 5 | OD1 | ASP | B | 277 | −7.208 | −61.774 | −29.439 | 1.00 | 63.05 | O |
| ATOM | 6 | OD2 | ASP | B | 277 | −5.723 | −60.356 | −28.670 | 1.00 | 63.07 | O |
| ATOM | 7 | C | ASP | B | 277 | −7.866 | −59.576 | −33.010 | 1.00 | 60.97 | C |
| ATOM | 8 | O | ASP | B | 277 | −8.984 | −59.023 | −33.065 | 1.00 | 61.14 | O |
| ATOM | 1 | N | LEU | B | 278 | −6.911 | −59.359 | −33.914 | 1.00 | 60.75 | N |
| ATOM | 2 | CA | LEU | B | 278 | −7.157 | −58.435 | −35.024 | 1.00 | 60.28 | C |
| ATOM | 3 | CB | LEU | B | 278 | −5.912 | −58.273 | −35.910 | 1.00 | 60.28 | C |
| ATOM | 4 | CG | LEU | B | 278 | −6.060 | −57.508 | −37.232 | 1.00 | 59.63 | C |
| ATOM | 5 | CD1 | LEU | B | 278 | −6.308 | −56.034 | −36.981 | 1.00 | 58.86 | C |
| ATOM | 6 | CD2 | LEU | B | 278 | −4.834 | −57.694 | −38.107 | 1.00 | 59.64 | C |
| ATOM | 7 | C | LEU | B | 278 | −8.360 | −58.894 | −35.849 | 1.00 | 60.34 | C |
| ATOM | 8 | O | LEU | B | 278 | −9.230 | −58.091 | −36.199 | 1.00 | 60.18 | O |
| ATOM | 1 | N | PHE | B | 279 | −8.416 | −60.190 | −36.148 | 1.00 | 60.47 | N |
| ATOM | 2 | CA | PHE | B | 279 | −9.530 | −60.706 | −36.933 | 1.00 | 60.57 | C |
| ATOM | 3 | CB | PHE | B | 279 | −9.397 | −62.197 | −37.221 | 1.00 | 60.52 | C |
| ATOM | 4 | CG | PHE | B | 279 | −8.381 | −62.511 | −38.265 | 1.00 | 60.50 | C |
| ATOM | 5 | CD1 | PHE | B | 279 | −7.966 | −61.533 | −39.154 | 1.00 | 60.16 | C |
| ATOM | 6 | CE1 | PHE | B | 279 | −7.023 | −61.808 | −40.117 | 1.00 | 60.38 | C |
| ATOM | 7 | CZ | PHE | B | 279 | −6.492 | −63.075 | −40.213 | 1.00 | 60.80 | C |
| ATOM | 8 | CE2 | PHE | B | 279 | −6.908 | −64.068 | −39.335 | 1.00 | 60.94 | C |
| ATOM | 9 | CD2 | PHE | B | 279 | −7.846 | −63.783 | −38.369 | 1.00 | 60.60 | C |
| ATOM | 10 | C | PHE | B | 279 | −10.850 | −60.418 | −36.253 | 1.00 | 60.72 | C |
| ATOM | 11 | O | PHE | B | 279 | −11.761 | −59.888 | −36.876 | 1.00 | 60.64 | O |
| ATOM | 1 | N | ARG | B | 280 | −10.962 | −60.758 | −34.975 | 1.00 | 60.96 | N |
| ATOM | 2 | CA | ARG | B | 280 | −12.162 | −60.380 | −34.250 | 1.00 | 61.16 | C |
| ATOM | 3 | CB | ARG | B | 280 | −12.049 | −60.714 | −32.760 | 1.00 | 61.05 | C |
| ATOM | 4 | CG | ARG | B | 280 | −12.708 | −62.041 | −32.395 | 1.00 | 61.56 | C |
| ATOM | 5 | CD | ARG | B | 280 | −12.596 | −62.359 | −30.907 | 1.00 | 61.94 | C |
| ATOM | 6 | NE | ARG | B | 280 | −11.469 | −63.243 | −30.603 | 1.00 | 63.84 | N |
| ATOM | 7 | CZ | ARG | B | 280 | −10.213 | −62.833 | −30.424 | 1.00 | 64.28 | C |
| ATOM | 8 | NH1 | ARG | B | 280 | −9.910 | −61.542 | −30.520 | 1.00 | 64.38 | N |
| ATOM | 9 | NH2 | ARG | B | 280 | −9.259 | −63.718 | −30.150 | 1.00 | 64.17 | N |
| ATOM | 10 | C | ARG | B | 280 | −12.451 | −58.895 | −34.487 | 1.00 | 61.05 | C |
| ATOM | 11 | O | ARG | B | 280 | −13.571 | −58.525 | −34.856 | 1.00 | 60.89 | O |
| ATOM | 1 | N | ASP | B | 281 | −11.432 | −58.053 | −34.322 | 1.00 | 61.18 | N |
| ATOM | 2 | CA | ASP | B | 281 | −11.623 | −56.605 | −34.494 | 1.00 | 61.26 | C |
| ATOM | 3 | CB | ASP | B | 281 | −10.314 | −55.846 | −34.241 | 1.00 | 61.67 | C |
| ATOM | 4 | CG | ASP | B | 281 | −10.523 | −54.555 | −33.450 | 1.00 | 62.92 | C |
| ATOM | 5 | OD1 | ASP | B | 281 | −11.661 | −54.026 | −33.430 | 1.00 | 63.95 | O |
| ATOM | 6 | OD2 | ASP | B | 281 | −9.541 | −54.076 | −32.835 | 1.00 | 64.41 | O |
| ATOM | 7 | C | ASP | B | 281 | −12.200 | −56.222 | −35.863 | 1.00 | 60.84 | C |
| ATOM | 8 | O | ASP | B | 281 | −13.146 | −55.446 | −35.940 | 1.00 | 60.76 | O |
| ATOM | 1 | N | ILE | B | 282 | −11.625 | −56.763 | −36.935 | 1.00 | 60.54 | N |
| ATOM | 2 | CA | ILE | B | 282 | −12.116 | −56.507 | −38.294 | 1.00 | 60.21 | C |
| ATOM | 3 | CB | ILE | B | 282 | −11.226 | −57.185 | −39.349 | 1.00 | 60.12 | C |
| ATOM | 4 | CG1 | ILE | B | 282 | −9.816 | −56.592 | −39.336 | 1.00 | 60.23 | C |
| ATOM | 5 | CD1 | ILE | B | 282 | −8.799 | −57.415 | −40.105 | 1.00 | 59.99 | C |
| ATOM | 6 | CG2 | ILE | B | 282 | −11.857 | −57.075 | −40.733 | 1.00 | 59.55 | C |
| ATOM | 7 | C | ILE | B | 282 | −13.509 | −57.077 | −38.474 | 1.00 | 60.29 | C |
| ATOM | 8 | O | ILE | B | 282 | −14.445 | −56.371 | −38.826 | 1.00 | 60.29 | O |
| ATOM | 1 | N | GLN | B | 283 | −13.628 | −58.375 | −38.232 | 1.00 | 60.51 | N |
| ATOM | 2 | CA | GLN | B | 283 | −14.874 | −59.096 | −38.404 | 1.00 | 60.54 | C |
| ATOM | 3 | CB | GLN | B | 283 | −14.718 | −60.538 | −37.919 | 1.00 | 60.59 | C |
| ATOM | 4 | CG | GLN | B | 283 | −15.750 | −61.500 | −38.479 | 1.00 | 61.30 | C |
| ATOM | 5 | CD | GLN | B | 283 | −16.834 | −61.830 | −37.479 | 1.00 | 62.16 | C |
| ATOM | 6 | OE1 | GLN | B | 283 | −18.015 | −61.936 | −37.829 | 1.00 | 62.37 | O |
| ATOM | 7 | NE2 | GLN | B | 283 | −16.438 | −61.995 | −36.219 | 1.00 | 62.82 | N |
| ATOM | 8 | C | GLN | B | 283 | −15.998 | −58.403 | −37.660 | 1.00 | 60.41 | C |
| ATOM | 9 | O | GLN | B | 283 | −17.160 | −58.514 | −38.045 | 1.00 | 60.58 | O |
| ATOM | 1 | N | GLY | B | 284 | −15.645 | −57.670 | −36.608 | 1.00 | 60.32 | N |
| ATOM | 2 | CA | GLY | B | 284 | −16.642 | −56.993 | −35.785 | 1.00 | 59.90 | C |
| ATOM | 3 | C | GLY | B | 284 | −17.147 | −55.656 | −36.307 | 1.00 | 59.63 | C |
| ATOM | 4 | O | GLY | B | 284 | −18.036 | −55.052 | −35.708 | 1.00 | 59.69 | O |
| ATOM | 1 | N | LEU | B | 285 | −16.596 | −55.194 | −37.425 | 1.00 | 59.39 | N |
| ATOM | 2 | CA | LEU | B | 285 | −16.897 | −53.848 | −37.933 | 1.00 | 59.12 | C |
| ATOM | 3 | CB | LEU | B | 285 | −15.994 | −53.498 | −39.122 | 1.00 | 58.97 | C |
| ATOM | 4 | CG | LEU | B | 285 | −14.509 | −53.305 | −38.827 | 1.00 | 58.44 | C |
| ATOM | 5 | CD1 | LEU | B | 285 | −13.791 | −52.805 | −40.064 | 1.00 | 58.10 | C |
| ATOM | 6 | CD2 | LEU | B | 285 | −14.322 | −52.345 | −37.673 | 1.00 | 58.23 | C |
| ATOM | 7 | C | LEU | B | 285 | −18.364 | −53.581 | −38.295 | 1.00 | 59.12 | C |
| ATOM | 8 | O | LEU | B | 285 | −18.966 | −52.638 | −37.773 | 1.00 | 58.98 | O |
| ATOM | 1 | N | PRO | B | 286 | −18.944 | −54.409 | −39.185 | 1.00 | 59.17 | N |
| ATOM | 2 | CA | PRO | B | 286 | −20.292 | −54.145 | −39.680 | 1.00 | 59.19 | C |
| ATOM | 3 | CB | PRO | B | 286 | −20.575 | −55.339 | −40.595 | 1.00 | 59.12 | C |
| ATOM | 4 | CG | PRO | B | 286 | −19.244 | −55.870 | −40.958 | 1.00 | 59.19 | C |
| ATOM | 5 | CD | PRO | B | 286 | −18.388 | −55.648 | −39.756 | 1.00 | 59.17 | C |
| ATOM | 6 | C | PRO | B | 286 | −21.289 | −54.132 | −38.541 | 1.00 | 59.26 | C |
| ATOM | 7 | O | PRO | B | 286 | −22.246 | −53.357 | −38.560 | 1.00 | 59.19 | O |
| ATOM | 1 | N | ARG | B | 287 | −21.053 | −54.989 | −37.552 | 1.00 | 59.46 | N |
| ATOM | 2 | CA | ARG | B | 287 | −21.912 | −55.057 | −36.384 | 1.00 | 59.64 | C |
| ATOM | 3 | CB | ARG | B | 287 | −21.297 | −55.921 | −35.291 | 1.00 | 59.76 | C |
| ATOM | 4 | CG | ARG | B | 287 | −22.268 | −56.326 | −34.193 | 1.00 | 60.10 | C |
| ATOM | 5 | CD | ARG | B | 287 | −21.647 | −57.427 | −33.373 | 1.00 | 61.21 | C |
| ATOM | 6 | NE | ARG | B | 287 | −20.970 | −58.384 | −34.246 | 1.00 | 62.03 | N |
| ATOM | 7 | CZ | ARG | B | 287 | −19.899 | −59.089 | −33.899 | 1.00 | 62.52 | C |
| ATOM | 8 | NH1 | ARG | B | 287 | −19.352 | −59.933 | −34.765 | 1.00 | 62.89 | N |
| ATOM | 9 | NH2 | ARG | B | 287 | −19.371 | −58.943 | −32.691 | 1.00 | 62.75 | N |
| ATOM | 10 | C | ARG | B | 287 | −22.185 | −53.659 | −35.866 | 1.00 | 59.65 | C |
| ATOM | 11 | O | ARG | B | 287 | −23.290 | −53.378 | −35.409 | 1.00 | 59.81 | O |
| ATOM | 1 | N | HIS | B | 288 | −21.194 | −52.772 | −35.937 | 1.00 | 59.63 | N |
| ATOM | 2 | CA | HIS | B | 288 | −21.517 | −51.366 | −35.722 | 1.00 | 59.67 | C |
| ATOM | 3 | CB | HIS | B | 288 | −21.830 | −51.042 | −34.267 | 1.00 | 59.99 | C |
| ATOM | 4 | CG | HIS | B | 288 | −23.288 | −50.816 | −34.035 | 1.00 | 61.01 | C |
| ATOM | 5 | ND1 | HIS | B | 288 | −24.084 | −50.164 | −34.954 | 1.00 | 61.71 | N |
| ATOM | 6 | CE1 | HIS | B | 288 | −25.326 | −50.118 | −34.502 | 1.00 | 62.63 | C |
| ATOM | 7 | NE2 | HIS | B | 288 | −25.364 | −50.719 | −33.325 | 1.00 | 62.85 | N |
| ATOM | 8 | CD2 | HIS | B | 288 | −24.103 | −51.171 | −33.013 | 1.00 | 62.15 | C |
| ATOM | 9 | C | HIS | B | 288 | −20.713 | −50.263 | −36.383 | 1.00 | 59.26 | C |
| ATOM | 10 | O | HIS | B | 288 | −19.713 | −49.774 | −35.856 | 1.00 | 59.07 | O |
| ATOM | 1 | N | ALA | B | 289 | −21.221 | −49.867 | −37.543 | 1.00 | 58.88 | N |
| ATOM | 2 | CA | ALA | B | 289 | −20.802 | −48.660 | −38.215 | 1.00 | 58.55 | C |
| ATOM | 3 | CB | ALA | B | 289 | −21.132 | −48.746 | −39.701 | 1.00 | 58.66 | C |
| ATOM | 4 | C | ALA | B | 289 | −21.513 | −47.468 | −37.582 | 1.00 | 58.24 | C |
| ATOM | 5 | O | ALA | B | 289 | −20.964 | −46.372 | −37.524 | 1.00 | 58.03 | O |
| ATOM | 1 | N | ALA | B | 290 | −22.733 | −47.688 | −37.102 | 1.00 | 57.82 | N |
| ATOM | 2 | CA | ALA | B | 290 | −23.524 | −46.609 | −36.525 | 1.00 | 57.55 | C |
| ATOM | 3 | CB | ALA | B | 290 | −24.807 | −47.154 | −35.945 | 1.00 | 57.56 | C |
| ATOM | 4 | C | ALA | B | 290 | −22.752 | −45.822 | −35.467 | 1.00 | 57.41 | C |
| ATOM | 5 | O | ALA | B | 290 | −22.784 | −44.587 | −35.446 | 1.00 | 57.25 | O |
| ATOM | 1 | N | LEU | B | 291 | −22.057 | −46.542 | −34.594 | 1.00 | 57.29 | N |
| ATOM | 2 | CA | LEU | B | 291 | −21.296 | −45.916 | −33.521 | 1.00 | 57.23 | C |
| ATOM | 3 | CB | LEU | B | 291 | −20.743 | −46.984 | −32.575 | 1.00 | 57.15 | C |
| ATOM | 4 | CG | LEU | B | 291 | −20.611 | −46.651 | −31.087 | 1.00 | 56.86 | C |
| ATOM | 5 | CD1 | LEU | B | 291 | −19.866 | −47.763 | −30.376 | 1.00 | 56.79 | C |
| ATOM | 6 | CD2 | LEU | B | 291 | −19.907 | −45.328 | −30.867 | 1.00 | 57.08 | C |
| ATOM | 7 | C | LEU | B | 291 | −20.158 | −45.059 | −34.081 | 1.00 | 57.31 | C |
| ATOM | 8 | O | LEU | B | 291 | −20.002 | −43.894 | −33.703 | 1.00 | 57.24 | O |
| ATOM | 1 | N | ARG | B | 292 | −19.366 | −45.629 | −34.986 | 1.00 | 57.43 | N |
| ATOM | 2 | CA | ARG | B | 292 | −18.224 | −44.901 | −35.540 | 1.00 | 57.57 | C |
| ATOM | 3 | CB | ARG | B | 292 | −17.290 | −45.826 | −36.338 | 1.00 | 57.62 | C |
| ATOM | 4 | CG | ARG | B | 292 | −17.564 | −45.912 | −37.834 | 1.00 | 57.76 | C |
| ATOM | 5 | CD | ARG | B | 292 | −16.564 | −45.093 | −38.644 | 1.00 | 57.94 | C |
| ATOM | 6 | NE | ARG | B | 292 | −16.928 | −45.049 | −40.060 | 1.00 | 58.09 | N |
| ATOM | 7 | CZ | ARG | B | 292 | −17.435 | −43.984 | −40.674 | 1.00 | 57.92 | C |
| ATOM | 8 | NH1 | ARG | B | 292 | −17.741 | −44.043 | −41.960 | 1.00 | 58.28 | N |
| ATOM | 9 | NH2 | ARG | B | 292 | −17.633 | −42.855 | −40.009 | 1.00 | 58.17 | N |
| ATOM | 10 | C | ARG | B | 292 | −18.704 | −43.718 | −36.375 | 1.00 | 57.46 | C |
| ATOM | 11 | O | ARG | B | 292 | −18.088 | −42.648 | −36.363 | 1.00 | 57.56 | O |
| ATOM | 1 | N | LYS | B | 293 | −19.812 | −43.912 | −37.081 | 1.00 | 57.36 | N |
| ATOM | 2 | CA | LYS | B | 293 | −20.442 | −42.831 | −37.816 | 1.00 | 57.19 | C |
| ATOM | 3 | CB | LYS | B | 293 | −21.686 | −43.323 | −38.553 | 1.00 | 57.19 | C |
| ATOM | 4 | CG | LYS | B | 293 | −21.386 | −44.029 | −39.863 | 1.00 | 57.17 | C |
| ATOM | 5 | CD | LYS | B | 293 | −22.550 | −44.901 | −40.305 | 1.00 | 57.02 | C |
| ATOM | 6 | CE | LYS | B | 293 | −22.257 | −45.577 | −41.635 | 1.00 | 57.19 | C |
| ATOM | 7 | NZ | LYS | B | 293 | −23.249 | −46.645 | −41.939 | 1.00 | 57.34 | N |
| ATOM | 8 | C | LYS | B | 293 | −20.804 | −41.729 | −36.840 | 1.00 | 57.09 | C |
| ATOM | 9 | O | LYS | B | 293 | −20.365 | −40.588 | −36.991 | 1.00 | 57.11 | O |
| ATOM | 1 | N | LEU | B | 294 | −21.591 | −42.079 | −35.828 | 1.00 | 56.95 | N |
| ATOM | 2 | CA | LEU | B | 294 | −21.958 | −41.121 | −34.800 | 1.00 | 56.84 | C |
| ATOM | 3 | CB | LEU | B | 294 | −22.623 | −41.836 | −33.624 | 1.00 | 56.79 | C |
| ATOM | 4 | CG | LEU | B | 294 | −22.941 | −40.974 | −32.400 | 1.00 | 56.79 | C |
| ATOM | 5 | CD1 | LEU | B | 294 | −23.835 | −39.809 | −32.786 | 1.00 | 57.30 | C |
| ATOM | 6 | CD2 | LEU | B | 294 | −23.595 | −41.802 | −31.315 | 1.00 | 56.83 | C |
| ATOM | 7 | C | LEU | B | 294 | −20.726 | −40.348 | −34.333 | 1.00 | 56.79 | C |
| ATOM | 8 | O | LEU | B | 294 | −20.667 | −39.112 | −34.429 | 1.00 | 56.76 | O |
| ATOM | 1 | N | ASN | B | 295 | −19.736 | −41.084 | −33.844 | 1.00 | 56.85 | N |
| ATOM | 2 | CA | ASN | B | 295 | −18.503 | −40.469 | −33.356 | 1.00 | 56.94 | C |
| ATOM | 3 | CB | ASN | B | 295 | −17.499 | −41.549 | −32.961 | 1.00 | 57.00 | C |
| ATOM | 4 | CG | ASN | B | 295 | −17.415 | −41.708 | −31.447 | 1.00 | 57.02 | C |
| ATOM | 5 | OD1 | ASN | B | 295 | −17.582 | −42.815 | −30.911 | 1.00 | 56.80 | O |
| ATOM | 6 | ND2 | ASN | B | 295 | −17.156 | −40.594 | −30.743 | 1.00 | 57.22 | N |
| ATOM | 7 | C | ASN | B | 295 | −17.855 | −39.494 | −34.338 | 1.00 | 56.98 | C |
| ATOM | 8 | O | ASN | B | 295 | −17.529 | −38.355 | −33.986 | 1.00 | 56.90 | O |
| ATOM | 1 | N | ASP | B | 296 | −17.663 | −39.939 | −35.572 | 1.00 | 57.20 | N |
| ATOM | 2 | CA | ASP | B | 296 | −17.044 | −39.075 | −36.564 | 1.00 | 57.27 | C |
| ATOM | 3 | CB | ASP | B | 296 | −16.740 | −39.854 | −37.843 | 1.00 | 57.49 | C |
| ATOM | 4 | CG | ASP | B | 296 | −15.732 | −40.978 | −37.613 | 1.00 | 58.34 | C |
| ATOM | 5 | OD1 | ASP | B | 296 | −15.736 | −41.950 | −38.398 | 1.00 | 59.28 | O |
| ATOM | 6 | OD2 | ASP | B | 296 | −14.941 | −40.898 | −36.642 | 1.00 | 59.32 | O |
| ATOM | 7 | C | ASP | B | 296 | −17.909 | −37.844 | −36.826 | 1.00 | 57.08 | C |
| ATOM | 8 | O | ASP | B | 296 | −17.386 | −36.745 | −37.049 | 1.00 | 56.96 | O |
| ATOM | 1 | N | LEU | B | 297 | −19.228 | −38.027 | −36.768 | 1.00 | 56.92 | N |
| ATOM | 2 | CA | LEU | B | 297 | −20.152 | −36.901 | −36.856 | 1.00 | 56.71 | C |
| ATOM | 3 | CB | LEU | B | 297 | −21.610 | −37.360 | −36.779 | 1.00 | 56.70 | C |
| ATOM | 4 | CG | LEU | B | 297 | −22.645 | −36.289 | −37.147 | 1.00 | 57.00 | C |
| ATOM | 5 | CD1 | LEU | B | 297 | −22.838 | −36.212 | −38.653 | 1.00 | 57.18 | C |
| ATOM | 6 | CD2 | LEU | B | 297 | −23.986 | −36.538 | −36.466 | 1.00 | 57.32 | C |
| ATOM | 7 | C | LEU | B | 297 | −19.857 | −35.904 | −35.743 | 1.00 | 56.57 | C |
| ATOM | 8 | O | LEU | B | 297 | −19.723 | −34.709 | −36.001 | 1.00 | 56.57 | O |
| ATOM | 1 | N | VAL | B | 298 | −19.746 | −36.394 | −34.509 | 1.00 | 56.40 | N |
| ATOM | 2 | CA | VAL | B | 298 | −19.372 | −35.528 | −33.385 | 1.00 | 56.20 | C |
| ATOM | 3 | CB | VAL | B | 298 | −19.201 | −36.323 | −32.082 | 1.00 | 56.10 | C |
| ATOM | 4 | CG1 | VAL | B | 298 | −18.713 | −35.414 | −30.963 | 1.00 | 55.78 | C |
| ATOM | 5 | CG2 | VAL | B | 298 | −20.494 | −37.011 | −31.700 | 1.00 | 55.98 | C |
| ATOM | 6 | C | VAL | B | 298 | −18.062 | −34.781 | −33.644 | 1.00 | 56.34 | C |
| ATOM | 7 | O | VAL | B | 298 | −17.990 | −33.550 | −33.511 | 1.00 | 56.51 | O |
| ATOM | 1 | N | LYS | B | 299 | −17.023 | −35.532 | −34.003 | 1.00 | 56.48 | N |
| ATOM | 2 | CA | LYS | B | 299 | −15.707 | −34.944 | −34.234 | 1.00 | 56.48 | C |
| ATOM | 3 | CB | LYS | B | 299 | −14.723 | −36.009 | −34.720 | 1.00 | 56.50 | C |
| ATOM | 4 | CG | LYS | B | 299 | −14.428 | −37.118 | −33.712 | 1.00 | 56.54 | C |
| ATOM | 5 | CD | LYS | B | 299 | −13.557 | −38.204 | −34.340 | 1.00 | 56.73 | C |
| ATOM | 6 | CE | LYS | B | 299 | −13.154 | −39.272 | −33.329 | 1.00 | 57.33 | C |
| ATOM | 7 | NZ | LYS | B | 299 | −12.203 | −38.749 | −32.312 | 1.00 | 57.04 | N |
| ATOM | 8 | C | LYS | B | 299 | −15.812 | −33.824 | −35.260 | 1.00 | 56.44 | C |
| ATOM | 9 | O | LYS | B | 299 | −15.372 | −32.676 | −35.023 | 1.00 | 56.62 | O |
| ATOM | 1 | N | ARG | B | 300 | −16.410 | −34.158 | −36.401 | 1.00 | 56.46 | N |
| ATOM | 2 | CA | ARG | B | 300 | −16.647 | −33.159 | −37.427 | 1.00 | 56.31 | C |
| ATOM | 3 | CB | ARG | B | 300 | −17.485 | −33.720 | −38.576 | 1.00 | 56.27 | C |
| ATOM | 4 | CG | ARG | B | 300 | −17.978 | −32.651 | −39.546 | 1.00 | 56.17 | C |
| ATOM | 5 | CD | ARG | B | 300 | −18.774 | −33.246 | −40.692 | 1.00 | 56.14 | C |
| ATOM | 6 | NE | ARG | B | 300 | −17.929 | −33.563 | −41.836 | 1.00 | 56.16 | N |
| ATOM | 7 | CZ | ARG | B | 300 | −18.030 | −32.970 | −43.021 | 1.00 | 56.53 | C |
| ATOM | 8 | NH1 | ARG | B | 300 | −17.222 | −33.317 | −44.013 | 1.00 | 56.30 | N |
| ATOM | 9 | NH2 | ARG | B | 300 | −18.950 | −32.037 | −43.218 | 1.00 | 56.98 | N |
| ATOM | 10 | C | ARG | B | 300 | −17.334 | −31.952 | −36.802 | 1.00 | 56.37 | C |
| ATOM | 11 | O | ARG | B | 300 | −16.829 | −30.834 | −36.887 | 1.00 | 56.39 | O |
| ATOM | 1 | N | ALA | B | 301 | −18.473 | −32.186 | −36.154 | 1.00 | 56.45 | N |
| ATOM | 2 | CA | ALA | B | 301 | −19.214 | −31.106 | −35.509 | 1.00 | 56.41 | C |
| ATOM | 3 | CB | ALA | B | 301 | −20.258 | −31.660 | −34.552 | 1.00 | 56.29 | C |
| ATOM | 4 | C | ALA | B | 301 | −18.261 | −30.172 | −34.779 | 1.00 | 56.46 | C |
| ATOM | 5 | O | ALA | B | 301 | −18.222 | −28.971 | −35.062 | 1.00 | 56.47 | O |
| ATOM | 1 | N | ARG | B | 302 | −17.480 | −30.728 | −33.858 | 1.00 | 56.55 | N |
| ATOM | 2 | CA | ARG | B | 302 | −16.535 | −29.923 | −33.075 | 1.00 | 56.77 | C |
| ATOM | 3 | CB | ARG | B | 302 | −15.760 | −30.818 | −32.106 | 1.00 | 56.86 | C |
| ATOM | 4 | CG | ARG | B | 302 | −16.666 | −31.414 | −31.043 | 1.00 | 57.73 | C |
| ATOM | 5 | CD | ARG | B | 302 | −16.137 | −32.710 | −30.479 | 1.00 | 59.51 | C |
| ATOM | 6 | NE | ARG | B | 302 | −15.204 | −32.490 | −29.380 | 1.00 | 61.12 | N |
| ATOM | 7 | CZ | ARG | B | 302 | −13.883 | −32.597 | −29.490 | 1.00 | 62.12 | C |
| ATOM | 8 | NH1 | ARG | B | 302 | −13.331 | −32.927 | −30.655 | 1.00 | 62.16 | N |
| ATOM | 9 | NH2 | ARG | B | 302 | −13.112 | −32.381 | −28.431 | 1.00 | 62.71 | N |
| ATOM | 10 | C | ARG | B | 302 | −15.593 | −29.087 | −33.951 | 1.00 | 56.68 | C |
| ATOM | 11 | O | ARG | B | 302 | −15.424 | −27.862 | −33.734 | 1.00 | 56.68 | O |
| ATOM | 1 | N | LEU | B | 303 | −15.003 | −29.735 | −34.956 | 1.00 | 56.67 | N |
| ATOM | 2 | CA | LEU | B | 303 | −14.163 | −28.990 | −35.902 | 1.00 | 56.63 | C |
| ATOM | 3 | CB | LEU | B | 303 | −13.583 | −29.913 | −36.973 | 1.00 | 56.59 | C |
| ATOM | 4 | CG | LEU | B | 303 | −12.682 | −29.195 | −37.981 | 1.00 | 56.67 | C |
| ATOM | 5 | CD1 | LEU | B | 303 | −11.413 | −28.699 | −37.301 | 1.00 | 56.50 | C |
| ATOM | 6 | CD2 | LEU | B | 303 | −12.344 | −30.106 | −39.150 | 1.00 | 57.07 | C |
| ATOM | 7 | C | LEU | B | 303 | −14.933 | −27.832 | −36.560 | 1.00 | 56.59 | C |
| ATOM | 8 | O | LEU | B | 303 | −14.471 | −26.677 | −36.576 | 1.00 | 56.69 | O |
| ATOM | 1 | N | VAL | B | 304 | −16.106 | −28.149 | −37.102 | 1.00 | 56.40 | N |
| ATOM | 2 | CA | VAL | B | 304 | −16.982 | −27.143 | −37.684 | 1.00 | 56.15 | C |
| ATOM | 3 | CB | VAL | B | 304 | −18.362 | −27.728 | −38.028 | 1.00 | 56.03 | C |
| ATOM | 4 | CG1 | VAL | B | 304 | −19.301 | −26.633 | −38.494 | 1.00 | 55.85 | C |
| ATOM | 5 | CG2 | VAL | B | 304 | −18.228 | −28.800 | −39.086 | 1.00 | 55.67 | C |
| ATOM | 6 | C | VAL | B | 304 | −17.155 | −25.971 | −36.724 | 1.00 | 56.22 | C |
| ATOM | 7 | O | VAL | B | 304 | −16.819 | −24.832 | −37.058 | 1.00 | 56.31 | O |
| ATOM | 1 | N | ARG | B | 305 | −17.666 | −26.249 | −35.527 | 1.00 | 56.24 | N |
| ATOM | 2 | CA | ARG | B | 305 | −17.864 | −25.195 | −34.532 | 1.00 | 56.28 | C |
| ATOM | 3 | CB | ARG | B | 305 | −18.260 | −25.783 | −33.169 | 1.00 | 56.25 | C |
| ATOM | 4 | CG | ARG | B | 305 | −17.825 | −24.961 | −31.964 | 1.00 | 56.53 | C |
| ATOM | 5 | CD | ARG | B | 305 | −18.985 | −24.659 | −31.011 | 1.00 | 57.09 | C |
| ATOM | 6 | NE | ARG | B | 305 | −19.403 | −25.757 | −30.137 | 1.00 | 57.25 | N |
| ATOM | 7 | CZ | ARG | B | 305 | −18.624 | −26.746 | −29.709 | 1.00 | 57.93 | C |
| ATOM | 8 | NH1 | ARG | B | 305 | −19.132 | −27.680 | −28.915 | 1.00 | 57.81 | N |
| ATOM | 9 | NH2 | ARG | B | 305 | −17.345 | −26.809 | −30.063 | 1.00 | 58.76 | N |
| ATOM | 10 | C | ARG | B | 305 | −16.626 | −24.303 | −34.435 | 1.00 | 56.28 | C |
| ATOM | 11 | O | ARG | B | 305 | −16.723 | −23.058 | −34.526 | 1.00 | 56.39 | O |
| ATOM | 1 | N | VAL | B | 306 | −15.456 | −24.931 | −34.294 | 1.00 | 56.35 | N |
| ATOM | 2 | CA | VAL | B | 306 | −14.227 | −24.136 | −34.253 | 1.00 | 56.27 | C |
| ATOM | 3 | CB | VAL | B | 306 | −12.987 | −25.017 | −34.201 | 1.00 | 56.15 | C |
| ATOM | 4 | CG1 | VAL | B | 306 | −11.767 | −24.162 | −33.924 | 1.00 | 56.18 | C |
| ATOM | 5 | CG2 | VAL | B | 306 | −13.152 | −26.085 | −33.145 | 1.00 | 56.17 | C |
| ATOM | 6 | C | VAL | B | 306 | −14.108 | −23.217 | −35.474 | 1.00 | 56.36 | C |
| ATOM | 7 | O | VAL | B | 306 | −14.036 | −21.983 | −35.351 | 1.00 | 56.51 | O |
| ATOM | 1 | N | HIS | B | 307 | −14.088 | −23.822 | −36.656 | 1.00 | 56.41 | N |
| ATOM | 2 | CA | HIS | B | 307 | −13.971 | −23.049 | −37.890 | 1.00 | 56.44 | C |
| ATOM | 3 | CB | HIS | B | 307 | −14.243 | −23.942 | −39.100 | 1.00 | 56.45 | C |
| ATOM | 4 | CG | HIS | B | 307 | −14.152 | −23.225 | −40.409 | 1.00 | 55.91 | C |
| ATOM | 5 | ND1 | HIS | B | 307 | −12.949 | −22.849 | −40.969 | 1.00 | 55.76 | N |
| ATOM | 6 | CE1 | HIS | B | 307 | −13.172 | −22.245 | −42.123 | 1.00 | 55.92 | C |
| ATOM | 7 | NE2 | HIS | B | 307 | −14.476 | −22.221 | −42.335 | 1.00 | 56.18 | N |
| ATOM | 8 | CD2 | HIS | B | 307 | −15.112 | −22.827 | −41.277 | 1.00 | 55.83 | C |
| ATOM | 9 | C | HIS | B | 307 | −14.927 | −21.856 | −37.902 | 1.00 | 56.54 | C |
| ATOM | 10 | O | HIS | B | 307 | −14.523 | −20.712 | −38.178 | 1.00 | 56.69 | O |
| ATOM | 1 | N | ALA | B | 308 | −16.196 | −22.138 | −37.602 | 1.00 | 56.50 | N |
| ATOM | 2 | CA | ALA | B | 308 | −17.222 | −21.106 | −37.541 | 1.00 | 56.41 | C |
| ATOM | 3 | CB | ALA | B | 308 | −18.500 | −21.659 | −36.962 | 1.00 | 56.25 | C |
| ATOM | 4 | C | ALA | B | 308 | −16.711 | −19.972 | −36.685 | 1.00 | 56.41 | C |
| ATOM | 5 | O | ALA | B | 308 | −16.653 | −18.819 | −37.132 | 1.00 | 56.62 | O |
| ATOM | 1 | N | TYR | B | 309 | −16.314 | −20.299 | −35.459 | 1.00 | 56.40 | N |
| ATOM | 2 | CA | TYR | B | 309 | −15.811 | −19.251 | −34.584 | 1.00 | 56.51 | C |
| ATOM | 3 | CB | TYR | B | 309 | −15.368 | −19.809 | −33.233 | 1.00 | 56.57 | C |
| ATOM | 4 | CG | TYR | B | 309 | −16.521 | −20.031 | −32.285 | 1.00 | 56.51 | C |
| ATOM | 5 | CD1 | TYR | B | 309 | −16.661 | −21.225 | −31.596 | 1.00 | 56.48 | C |
| ATOM | 6 | CE1 | TYR | B | 309 | −17.719 | −21.429 | −30.733 | 1.00 | 56.59 | C |
| ATOM | 7 | CZ | TYR | B | 309 | −18.659 | −20.436 | −30.560 | 1.00 | 56.61 | C |
| ATOM | 8 | OH | TYR | B | 309 | −19.715 | −20.639 | −29.705 | 1.00 | 56.68 | O |
| ATOM | 9 | CE2 | TYR | B | 309 | −18.548 | −19.243 | −31.239 | 1.00 | 56.61 | C |
| ATOM | 10 | CD2 | TYR | B | 309 | −17.485 | −19.048 | −32.097 | 1.00 | 56.59 | C |
| ATOM | 11 | C | TYR | B | 309 | −14.693 | −18.455 | −35.251 | 1.00 | 56.61 | C |
| ATOM | 12 | O | TYR | B | 309 | −14.750 | −17.219 | −35.302 | 1.00 | 56.91 | O |
| ATOM | 1 | N | ILE | B | 310 | −13.693 | −19.158 | −35.781 | 1.00 | 56.59 | N |
| ATOM | 2 | CA | ILE | B | 310 | −12.568 | −18.486 | −36.444 | 1.00 | 56.63 | C |
| ATOM | 3 | CB | ILE | B | 310 | −11.613 | −19.492 | −37.109 | 1.00 | 56.52 | C |
| ATOM | 4 | CG1 | ILE | B | 310 | −11.052 | −20.463 | −36.074 | 1.00 | 56.47 | C |
| ATOM | 5 | CD1 | ILE | B | 310 | −10.286 | −21.610 | −36.681 | 1.00 | 56.57 | C |
| ATOM | 6 | CG2 | ILE | B | 310 | −10.480 | −18.768 | −37.804 | 1.00 | 56.40 | C |
| ATOM | 7 | C | ILE | B | 310 | −13.023 | −17.463 | −37.496 | 1.00 | 56.78 | C |
| ATOM | 8 | O | ILE | B | 310 | −12.692 | −16.258 | −37.410 | 1.00 | 57.05 | 0 |
| ATOM | 1 | N | ILE | B | 311 | −13.783 | −17.938 | −38.484 | 1.00 | 56.75 | N |
| ATOM | 2 | CA | ILE | B | 311 | −14.250 | −17.046 | −39.545 | 1.00 | 56.82 | C |
| ATOM | 3 | CB | ILE | B | 311 | −15.107 | −17.771 | −40.602 | 1.00 | 56.78 | C |
| ATOM | 4 | CG1 | ILE | B | 311 | −14.243 | −18.232 | −41.774 | 1.00 | 56.78 | C |
| ATOM | 5 | CD1 | ILE | B | 311 | −13.214 | −19.266 | −41.422 | 1.00 | 57.40 | C |
| ATOM | 6 | CG2 | ILE | B | 311 | −16.155 | −16.834 | −41.167 | 1.00 | 57.09 | C |
| ATOM | 7 | C | ILE | B | 311 | −15.032 | −15.882 | −38.951 | 1.00 | 56.87 | C |
| ATOM | 8 | O | ILE | B | 311 | −14.757 | −14.714 | −39.253 | 1.00 | 56.99 | O |
| ATOM | 1 | N | SER | B | 312 | −15.995 | −16.205 | −38.091 | 1.00 | 56.92 | N |
| ATOM | 2 | CA | SER | B | 312 | −16.804 | −15.181 | −37.447 | 1.00 | 57.06 | C |
| ATOM | 3 | CB | SER | B | 312 | −17.681 | −15.797 | −36.362 | 1.00 | 57.04 | C |
| ATOM | 4 | OG | SER | B | 312 | −18.624 | −16.687 | −36.931 | 1.00 | 57.03 | 0 |
| ATOM | 5 | C | SER | B | 312 | −15.937 | −14.078 | −36.855 | 1.00 | 57.22 | C |
| ATOM | 6 | O | SER | B | 312 | −16.133 | −12.896 | −37.157 | 1.00 | 57.33 | 0 |
| ATOM | 1 | N | TYR | B | 313 | −14.974 | −14.464 | −36.019 | 1.00 | 57.40 | N |
| ATOM | 2 | CA | TYR | B | 313 | −14.091 | −13.479 | −35.382 | 1.00 | 57.83 | C |
| ATOM | 3 | CB | TYR | B | 313 | −13.138 | −14.142 | −34.380 | 1.00 | 58.15 | C |
| ATOM | 4 | CG | TYR | B | 313 | −13.757 | −14.429 | −33.019 | 1.00 | 58.84 | C |
| ATOM | 5 | CD1 | TYR | B | 313 | −13.076 | −14.120 | −31.842 | 1.00 | 59.36 | C |
| ATOM | 6 | CE1 | TYR | B | 313 | −11.637 | −14.383 | −30.593 | 1.00 | 59.39 | C |
| ATOM | 7 | CZ | TYR | B | 313 | −14.895 | −14.957 | −30.515 | 1.00 | 59.16 | C |
| ATOM | 8 | OH | TYR | B | 313 | −15.457 | −15.219 | −29.286 | 1.00 | 59.00 | 0 |
| ATOM | 9 | CE2 | TYR | B | 313 | −15.593 | −15.269 | −31.667 | 1.00 | 59.29 | C |
| ATOM | 10 | CD2 | TYR | B | 313 | −15.025 | −15.004 | −32.909 | 1.00 | 59.34 | C |
| ATOM | 11 | C | TYR | B | 313 | −13.315 | −12.657 | −36.412 | 1.00 | 57.75 | C |
| ATOM | 12 | O | TYR | B | 313 | −13.234 | −11.423 | −36.305 | 1.00 | 57.91 | 0 |
| ATOM | 1 | N | LEU | B | 314 | −12.757 | −13.328 | −37.418 | 1.00 | 57.51 | N |
| ATOM | 2 | CA | LEU | B | 314 | −12.069 | −12.590 | −38.480 | 1.00 | 57.40 | C |
| ATOM | 3 | CB | LEU | B | 314 | −11.557 | −13.550 | −39.553 | 1.00 | 57.20 | C |
| ATOM | 4 | CG | LEU | B | 314 | −10.394 | −14.430 | −39.093 | 1.00 | 56.64 | C |
| ATOM | 5 | CD1 | LEU | B | 314 | −10.239 | −15.648 | −39.978 | 1.00 | 55.97 | C |
| ATOM | 6 | CD2 | LEU | B | 314 | −9.112 | −13.622 | −39.047 | 1.00 | 56.28 | C |
| ATOM | 7 | C | LEU | B | 314 | −12.981 | −11.514 | −39.088 | 1.00 | 57.49 | C |
| ATOM | 8 | O | LEU | B | 314 | −12.638 | −10.316 | −39.130 | 1.00 | 57.84 | O |
| ATOM | 1 | N | LYS | B | 315 | −14.156 | −11.949 | −39.539 | 1.00 | 57.46 | N |
| ATOM | 2 | CA | LYS | B | 315 | −15.124 | −11.045 | −40.158 | 1.00 | 57.51 | C |
| ATOM | 3 | CB | LYS | B | 315 | −16.380 | −11.804 | −40.593 | 1.00 | 57.50 | C |
| ATOM | 4 | CG | LYS | B | 315 | −17.579 | −10.920 | −40.897 | 1.00 | 57.23 | C |
| ATOM | 5 | CD | LYS | B | 315 | −17.328 | −10.022 | −42.090 | 1.00 | 57.26 | C |
| ATOM | 6 | CE | LYS | B | 315 | −18.512 | −9.103 | −42.327 | 1.00 | 57.59 | C |
| ATOM | 7 | NZ | LYS | B | 315 | −18.458 | −8.446 | −43.665 | 1.00 | 57.78 | N |
| ATOM | 8 | C | LYS | B | 315 | −15.503 | −9.898 | −39.235 | 1.00 | 57.60 | C |
| ATOM | 9 | O | LYS | B | 315 | −15.604 | −8.753 | −39.676 | 1.00 | 57.65 | 0 |
| ATOM | 1 | N | LYS | B | 316 | −15.717 | −10.202 | −37.958 | 1.00 | 57.67 | N |
| ATOM | 2 | CA | LYS | B | 316 | −16.071 | −9.156 | −37.003 | 1.00 | 57.96 | C |
| ATOM | 3 | CB | LYS | B | 316 | −16.594 | −9.747 | −35.685 | 1.00 | 58.04 | C |
| ATOM | 4 | CG | LYS | B | 316 | −15.533 | −10.014 | −34.617 | 1.00 | 58.13 | C |
| ATOM | 5 | CD | LYS | B | 316 | −16.133 | −10.748 | −33.420 | 1.00 | 58.14 | C |
| ATOM | 6 | CE | LYS | B | 316 | −15.059 | −11.212 | −32.442 | 1.00 | 58.37 | C |
| ATOM | 7 | NZ | LYS | B | 316 | −14.405 | −10.080 | −31.724 | 1.00 | 58.37 | N |
| ATOM | 8 | C | LYS | B | 316 | −14.886 | −8.223 | −36.755 | 1.00 | 57.99 | C |
| ATOM | 9 | O | LYS | B | 316 | −15.059 | −7.095 | −36.293 | 1.00 | 57.89 | O |
| ATOM | 1 | N | GLU | B | 317 | −13.683 | −8.693 | −37.071 | 1.00 | 58.12 | N |
| ATOM | 2 | CA | GLU | B | 317 | −12.504 | −7.848 | −36.916 | 1.00 | 58.35 | C |
| ATOM | 3 | CB | GLU | B | 317 | −11.252 | −8.689 | −36.655 | 1.00 | 58.34 | C |
| ATOM | 4 | CG | GLU | B | 317 | −10.222 | −7.983 | −35.796 | 1.00 | 58.29 | C |
| ATOM | 5 | CD | GLU | B | 317 | −10.786 | −7.568 | −34.449 | 1.00 | 58.49 | C |
| ATOM | 6 | OE1 | GLU | B | 317 | −10.392 | −6.495 | −33.940 | 1.00 | 58.78 | O |
| ATOM | 7 | OE2 | GLU | B | 317 | −11.630 | −8.311 | −33.901 | 1.00 | 58.20 | O |
| ATOM | 8 | C | GLU | B | 317 | −12.291 | −6.929 | −38.118 | 1.00 | 58.52 | C |
| ATOM | 9 | O | GLU | B | 317 | −11.812 | −5.806 | −37.964 | 1.00 | 58.43 | O |
| ATOM | 1 | N | MET | B | 318 | −12.647 | −7.408 | −39.309 | 1.00 | 58.76 | N |
| ATOM | 2 | CA | MET | B | 318 | −12.457 | −6.621 | −40.544 | 1.00 | 59.38 | C |
| ATOM | 3 | CB | MET | B | 318 | −12.850 | −7.457 | −41.761 | 1.00 | 59.35 | C |
| ATOM | 4 | CG | MET | B | 318 | −11.790 | −8.450 | −42.180 | 1.00 | 59.35 | C |
| ATOM | 5 | SD | MET | B | 318 | −10.352 | −7.627 | −42.883 | 1.00 | 59.21 | S |
| ATOM | 6 | CE | MET | B | 318 | −11.089 | −6.840 | −44.315 | 1.00 | 59.36 | C |
| ATOM | 7 | C | MET | B | 318 | −13.194 | −5.273 | −40.591 | 1.00 | 59.81 | C |
| ATOM | 8 | O | MET | B | 318 | −14.370 | −5.198 | −40.242 | 1.00 | 59.90 | O |
| ATOM | 1 | N | PRO | B | 319 | −12.501 | −4.208 | −41.048 | 1.00 | 60.27 | N |
| ATOM | 2 | CA | PRO | B | 319 | −13.070 | −2.859 | −41.164 | 1.00 | 60.66 | C |
| ATOM | 3 | CB | PRO | B | 319 | −11.830 | −1.968 | −41.230 | 1.00 | 60.60 | C |
| ATOM | 4 | CG | PRO | B | 319 | −10.811 | −2.817 | −41.904 | 1.00 | 60.41 | C |
| ATOM | 5 | CD | PRO | B | 319 | −11.097 | −4.248 | −41.497 | 1.00 | 60.27 | C |
| ATOM | 6 | C | PRO | B | 319 | −13.898 | −2.698 | −42.437 | 1.00 | 61.12 | C |
| ATOM | 7 | O | PRO | B | 319 | −13.536 | −3.243 | −43.484 | 1.00 | 61.23 | O |
| ATOM | 1 | N | THR | B | 320 | −14.988 | −1.936 | −42.346 | 1.00 | 61.63 | N |
| ATOM | 2 | CA | THR | B | 320 | −15.997 | −1.880 | −43.413 | 1.00 | 62.20 | C |
| ATOM | 3 | CB | THR | B | 320 | −17.120 | −0.850 | −43.106 | 1.00 | 62.25 | C |
| ATOM | 4 | OG1 | THR | B | 320 | −16.676 | 0.476 | −43.428 | 1.00 | 62.39 | O |
| ATOM | 5 | CG2 | THR | B | 320 | −17.533 | −0.914 | −41.636 | 1.00 | 62.52 | C |
| ATOM | 6 | C | THR | B | 320 | −15.446 | −1.611 | −44.818 | 1.00 | 62.49 | C |
| ATOM | 7 | O | THR | B | 320 | −15.468 | −2.492 | −45.683 | 1.00 | 62.58 | O |
| ATOM | 1 | N | VAL | B | 321 | −14.951 | −0.398 | −45.043 | 1.00 | 62.79 | N |
| ATOM | 2 | CA | VAL | B | 321 | −14.629 | 0.029 | −41.401 | 1.00 | 63.06 | C |
| ATOM | 3 | CB | VAL | B | 321 | −15.352 | 1.360 | −46.761 | 1.00 | 63.09 | C |
| ATOM | 4 | CG1 | VAL | B | 321 | −15.052 | 2.457 | −45.687 | 1.00 | 63.09 | C |
| ATOM | 5 | CG2 | VAL | B | 321 | −14.989 | 1.830 | −48.206 | 1.00 | 63.21 | C |
| ATOM | 6 | C | VAL | B | 321 | −13.125 | 0.097 | −46.726 | 1.00 | 63.21 | C |
| ATOM | 7 | O | VAL | B | 321 | −12.677 | −0.527 | −47.693 | 1.00 | 63.23 | O |
| ATOM | 1 | N | PHE | B | 322 | −12.346 | 0.829 | −45.930 | 1.00 | 63.40 | N |
| ATOM | 2 | CA | PHE | B | 322 | −10.911 | 0.966 | −46.222 | 1.00 | 63.56 | C |
| ATOM | 3 | CB | PHE | B | 322 | −10.553 | 2.392 | −46.676 | 1.00 | 63.65 | C |
| ATOM | 4 | CG | PHE | B | 322 | −11.291 | 3.475 | −45.943 | 1.00 | 63.76 | C |
| ATOM | 5 | CD1 | PHE | B | 322 | −12.129 | 4.341 | −46.630 | 1.00 | 63.84 | C |
| ATOM | 6 | CE1 | PHE | B | 322 | −12.811 | 5.344 | −45.964 | 1.00 | 64.14 | C |
| ATOM | 7 | CZ | PHE | B | 322 | −12.657 | 5.492 | −44.594 | 1.00 | 64.13 | C |
| ATOM | 8 | CE2 | PHE | B | 322 | −11.822 | 4.636 | −43.898 | 1.00 | 64.19 | C |
| ATOM | 9 | CD2 | PHE | B | 322 | −11.143 | 3.633 | −44.572 | 1.00 | 64.04 | C |
| ATOM | 10 | C | PHE | B | 322 | −9.959 | 0.500 | −45.119 | 1.00 | 63.57 | C |
| ATOM | 11 | O | PHE | B | 322 | −10.289 | 0.536 | −43.932 | 1.00 | 63.52 | O |
| ATOM | 1 | N | GLY | B | 323 | −8.771 | 0.074 | −45.542 | 1.00 | 63.63 | N |
| ATOM | 2 | CA | GLY | B | 323 | −7.757 | −0.470 | −44.645 | 1.00 | 63.75 | C |
| ATOM | 3 | C | GLY | B | 323 | −7.631 | −1.967 | −44.841 | 1.00 | 63.84 | C |
| ATOM | 4 | O | GLY | B | 323 | −6.624 | −2.574 | −44.481 | 1.00 | 63.82 | O |
| ATOM | 1 | N | LYS | B | 324 | −8.668 | −2.547 | −45.435 | 1.00 | 63.98 | N |
| ATOM | 2 | CA | LYS | B | 324 | −8.803 | −3.993 | −45.634 | 1.00 | 64.15 | C |
| ATOM | 3 | CB | LYS | B | 324 | −9.766 | −4.263 | −46.799 | 1.00 | 64.21 | C |
| ATOM | 4 | CG | LYS | B | 324 | −9.836 | −3.122 | −47.823 | 1.00 | 64.51 | C |
| ATOM | 5 | CD | LYS | B | 324 | −10.857 | −3.392 | −48.931 | 1.00 | 64.43 | C |
| ATOM | 6 | CE | LYS | B | 324 | −12.196 | −3.883 | −48.375 | 1.00 | 64.92 | C |
| ATOM | 7 | NZ | LYS | B | 324 | −12.780 | −2.994 | −47.323 | 1.00 | 64.78 | N |
| ATOM | 8 | C | LYS | B | 324 | −7.512 | −4.807 | −45.813 | 1.00 | 64.09 | C |
| ATOM | 9 | O | LYS | B | 324 | −7.325 | −5.828 | −45.148 | 1.00 | 64.04 | O |
| ATOM | 1 | N | GLU | B | 325 | −6.632 | −4.361 | −46.706 | 1.00 | 64.10 | N |
| ATOM | 2 | CA | GLU | B | 325 | −5.461 | −5.152 | −47.102 | 1.00 | 64.12 | C |
| ATOM | 3 | CB | GLU | B | 325 | −4.669 | −4.416 | −48.191 | 1.00 | 64.09 | C |
| ATOM | 4 | CG | GLU | B | 325 | −5.490 | −4.058 | −49.424 | 1.00 | 63.96 | C |
| ATOM | 5 | CD | GLU | B | 325 | −4.531 | −4.963 | −49.990 | 0.00 | 50.00 | C |
| ATOM | 6 | OE1 | GLU | B | 325 | −4.590 | −5.825 | −49.100 | 0.00 | 50.00 | O |
| ATOM | 7 | OE2 | GLU | B | 325 | −4.164 | −5.171 | −51.156 | 0.00 | 50.00 | O |
| ATOM | 8 | C | GLU | B | 325 | −4.530 | −5.549 | −45.942 | 1.00 | 64.19 | C |
| ATOM | 9 | O | GLU | B | 325 | −4.310 | −6.781 | −45.669 | 1.00 | 64.25 | O |
| ATOM | 1 | N | ASN | B | 326 | −3.972 | −4.524 | −45.270 | 1.00 | 64.26 | N |
| ATOM | 2 | CA | ASN | B | 326 | −3.005 | −4.812 | −44.204 | 1.00 | 64.33 | C |
| ATOM | 3 | CB | ASN | B | 326 | −2.040 | −3.633 | −43.942 | 1.00 | 64.30 | C |
| ATOM | 4 | CG | ASN | B | 326 | −2.752 | −2.335 | −43.604 | 1.00 | 64.30 | C |
| ATOM | 5 | OD1 | ASN | B | 326 | −2.563 | −1.317 | −44.277 | 1.00 | 64.17 | O |
| ATOM | 6 | ND2 | ASN | B | 326 | −3.558 | −2.357 | −42.545 | 1.00 | 64.50 | N |
| ATOM | 7 | C | ASN | B | 326 | −3.681 | −5.330 | −42.925 | 1.00 | 64.35 | C |
| ATOM | 8 | O | ASN | B | 326 | −3.046 | −5.991 | −42.099 | 1.00 | 64.40 | O |
| ATOM | 1 | N | LYS | B | 327 | −4.972 | −5.038 | −42.779 | 1.00 | 64.30 | N |
| ATOM | 2 | CA | LYS | B | 327 | −5.744 | −5.569 | −41.664 | 1.00 | 64.23 | C |
| ATOM | 3 | CB | LYS | B | 327 | −7.138 | −4.943 | −41.602 | 1.00 | 64.22 | C |
| ATOM | 4 | CG | LYS | B | 327 | −7.630 | −4.643 | −40.184 | 1.00 | 64.25 | C |
| ATOM | 5 | CD | LYS | B | 327 | −7.454 | −5.831 | −39.244 | 1.00 | 64.27 | C |
| ATOM | 6 | CE | LYS | B | 327 | −7.613 | −5.426 | −37.784 | 1.00 | 64.19 | C |
| ATOM | 7 | NZ | LYS | B | 327 | −9.010 | −5.015 | −37.462 | 1.00 | 64.30 | N |
| ATOM | 8 | C | LYS | B | 327 | −5.855 | −7.072 | −41.841 | 1.00 | 64.18 | C |
| ATOM | 9 | O | LYS | B | 327 | −5.758 | −7.825 | −40.879 | 1.00 | 64.23 | O |
| ATOM | 1 | N | LYS | B | 328 | −6.051 | −7.503 | −43.082 | 1.00 | 64.17 | N |
| ATOM | 2 | CA | LYS | B | 328 | −6.073 | −8.924 | −43.393 | 1.00 | 64.23 | C |
| ATOM | 3 | CB | LYS | B | 328 | −6.505 | −9.156 | −44.843 | 1.00 | 64.21 | C |
| ATOM | 4 | CG | LYS | B | 328 | −6.492 | −10.616 | −45.263 | 1.00 | 64.08 | C |
| ATOM | 5 | CD | LYS | B | 328 | −6.561 | −10.764 | −46.773 | 1.00 | 64.01 | C |
| ATOM | 6 | CE | LYS | B | 328 | −6.135 | −12.160 | −47.201 | 1.00 | 63.97 | C |
| ATOM | 7 | NZ | LYS | B | 328 | −6.027 | −12.286 | −48.679 | 1.00 | 63.96 | N |
| ATOM | 8 | C | LYS | B | 328 | −4.697 | −9.536 | −43.139 | 1.00 | 64.29 | C |
| ATOM | 9 | O | LYS | B | 328 | −4.569 | −10.507 | −42.366 | 1.00 | 64.33 | O |
| ATOM | 1 | N | LYS | B | 329 | −3.665 | −8.960 | −43.772 | 1.00 | 64.32 | N |
| ATOM | 2 | CA | LYS | B | 329 | −2.313 | −9.511 | −43.610 | 1.00 | 64.39 | C |
| ATOM | 3 | CB | LYS | B | 329 | −1.288 | −8.659 | −44.360 | 1.00 | 64.37 | C |
| ATOM | 4 | CG | LYS | B | 329 | −1.188 | −10.172 | −46.080 | 0.00 | 50.00 | C |
| ATOM | 5 | CD | LYS | B | 329 | −0.493 | −9.545 | −47.260 | 0.00 | 50.00 | C |
| ATOM | 6 | CE | LYS | B | 329 | 0.417 | −10.565 | −47.946 | 0.00 | 50.00 | C |
| ATOM | 7 | NZ | LYS | B | 329 | 0.748 | −10.177 | −49.338 | 0.00 | 50.00 | N |
| ATOM | 8 | C | LYS | B | 329 | −1.934 | −9.631 | −42.126 | 1.00 | 64.44 | C |
| ATOM | 9 | O | LYS | B | 329 | −1.435 | −10.671 | −41.665 | 1.00 | 64.52 | O |
| ATOM | 1 | N | GLN | B | 330 | −2.189 | −8.557 | −41.385 | 1.00 | 64.44 | N |
| ATOM | 2 | CA | GLN | B | 330 | −1.958 | −8.520 | −39.947 | 1.00 | 64.45 | C |
| ATOM | 3 | CB | GLN | B | 330 | −2.258 | −7.118 | −39.410 | 1.00 | 64.44 | C |
| ATOM | 4 | CG | GLN | B | 330 | −2.343 | −7.007 | −37.897 | 1.00 | 64.39 | C |
| ATOM | 5 | CD | GLN | B | 330 | −2.721 | −5.608 | −37.443 | 1.00 | 64.40 | C |
| ATOM | 6 | OE1 | GLN | B | 330 | −2.277 | −4.614 | −38.021 | 1.00 | 64.30 | O |
| ATOM | 7 | NE2 | GLN | B | 330 | −3.546 | −5.524 | −36.404 | 1.00 | 64.34 | N |
| ATOM | 8 | C | GLN | B | 330 | −2.805 | −9.565 | −39.225 | 1.00 | 64.45 | C |
| ATOM | 9 | O | GLN | B | 330 | −2.313 | −10.287 | −38.354 | 1.00 | 64.60 | O |
| ATOM | 1 | N | LEU | B | 331 | −4.079 | −9.647 | −39.602 | 1.00 | 64.39 | N |
| ATOM | 2 | CA | LEU | B | 331 | −4.995 | −10.615 | −39.009 | 1.00 | 64.37 | C |
| ATOM | 3 | CB | LEU | B | 331 | −6.402 | −10.488 | −39.599 | 1.00 | 64.32 | C |
| ATOM | 4 | CG | LEU | B | 331 | −7.355 | −9.519 | −38.897 | 1.00 | 64.30 | C |
| ATOM | 5 | CD1 | LEU | B | 331 | −8.678 | −9.421 | −39.641 | 1.00 | 64.35 | C |
| ATOM | 6 | CD2 | LEU | B | 331 | −7.581 | −9.930 | −37.453 | 1.00 | 64.38 | C |
| ATOM | 7 | C | LEU | B | 331 | −4.483 | −12.039 | −39.165 | 1.00 | 64.41 | C |
| ATOM | 8 | O | LEU | B | 331 | −4.671 | −12.868 | −38.273 | 1.00 | 64.39 | O |
| ATOM | 1 | N | ILE | B | 332 | −3.835 | −12.331 | −40.290 | 1.00 | 64.44 | N |
| ATOM | 2 | CA | ILE | B | 332 | −3.244 | −13.660 | −40.445 | 1.00 | 64.56 | C |
| ATOM | 3 | CB | ILE | B | 332 | −3.234 | −14.168 | −41.905 | 1.00 | 64.53 | C |
| ATOM | 4 | CG1 | ILE | B | 332 | −2.752 | −13.081 | −42.861 | 1.00 | 64.67 | C |
| ATOM | 5 | CD1 | ILE | B | 332 | −3.018 | −13.404 | −44.313 | 1.00 | 64.93 | C |
| ATOM | 6 | CG2 | ILE | B | 332 | −4.622 | −14.638 | −42.310 | 1.00 | 64.59 | C |
| ATOM | 7 | C | ILE | B | 332 | −1.861 | −13.779 | −39.807 | 1.00 | 64.64 | C |
| ATOM | 8 | O | ILE | B | 332 | −1.416 | −14.885 | −39.502 | 1.00 | 64.71 | O |
| ATOM | 1 | N | LEU | B | 333 | −1.181 | −12.653 | −39.597 | 1.00 | 64.69 | N |
| ATOM | 2 | CA | LEU | B | 333 | 0.026 | −12.679 | −38.763 | 1.00 | 64.86 | C |
| ATOM | 3 | CB | LEU | B | 333 | 0.767 | −11.340 | −38.813 | 1.00 | 64.84 | C |
| ATOM | 4 | CG | LEU | B | 333 | 2.123 | −11.329 | −39.526 | 1.00 | 64.83 | C |
| ATOM | 5 | CD1 | LEU | B | 333 | 1.995 | −11.795 | −40.971 | 1.00 | 64.93 | C |
| ATOM | 6 | CD2 | LEU | B | 333 | 2.750 | −9.944 | −39.452 | 1.00 | 64.92 | C |
| ATOM | 7 | C | LEU | B | 333 | −0.321 | −13.018 | −37.315 | 1.00 | 64.95 | C |
| ATOM | 8 | O | LEU | B | 333 | 0.434 | −13.699 | −36.618 | 1.00 | 64.92 | O |
| ATOM | 1 | N | LYS | B | 334 | −1.482 | −12.542 | −36.881 | 1.00 | 65.06 | N |
| ATOM | 2 | CA | LYS | B | 334 | −1.910 | −12.645 | −35.493 | 1.00 | 65.32 | C |
| ATOM | 3 | CB | LYS | B | 334 | −2.537 | −11.303 | −35.082 | 1.00 | 65.31 | C |
| ATOM | 4 | CG | LYS | B | 334 | −3.359 | −11.279 | −33.799 | 1.00 | 65.31 | C |
| ATOM | 5 | CD | LYS | B | 334 | −4.251 | −10.026 | −33.782 | 1.00 | 65.18 | C |
| ATOM | 6 | CE | LYS | B | 334 | −5.325 | −10.098 | −32.705 | 1.00 | 64.74 | C |
| ATOM | 7 | NZ | LYS | B | 334 | −6.339 | −9.010 | −32.867 | 1.00 | 64.46 | N |
| ATOM | 8 | C | LYS | B | 334 | −2.885 | −13.813 | −35.294 | 1.00 | 65.54 | C |
| ATOM | 9 | O | LYS | B | 334 | −3.741 | −13.778 | −34.409 | 1.00 | 65.57 | O |
| ATOM | 1 | N | LEU | B | 335 | −2.743 | −14.855 | −36.111 | 1.00 | 65.83 | N |
| ATOM | 2 | CA | LEU | B | 335 | −3.676 | −15.984 | −36.068 | 1.00 | 66.16 | C |
| ATOM | 3 | CB | LEU | B | 335 | −3.592 | −16.826 | −37.346 | 1.00 | 66.09 | C |
| ATOM | 4 | CG | LEU | B | 335 | −4.906 | −17.326 | −37.966 | 1.00 | 66.13 | C |
| ATOM | 5 | CD1 | LEU | B | 335 | −4.704 | −18.673 | −38.651 | 1.00 | 66.05 | C |
| ATOM | 6 | CD2 | LEU | B | 335 | −6.036 | −17.420 | −36.952 | 1.00 | 65.95 | C |
| ATOM | 7 | C | LEU | B | 335 | −3.490 | −16.877 | −34.839 | 1.00 | 66.47 | C |
| ATOM | 8 | O | LEU | B | 335 | −4.464 | −17.217 | −34.173 | 1.00 | 66.51 | O |
| ATOM | 1 | N | PRO | B | 336 | −2.239 | −17.263 | −34.529 | 1.00 | 66.81 | N |
| ATOM | 2 | CA | PRO | B | 336 | −2.044 | −18.144 | −33.380 | 1.00 | 67.08 | C |
| ATOM | 3 | CB | PRO | B | 336 | −0.528 | −18.367 | −33.357 | 1.00 | 67.05 | C |
| ATOM | 4 | CG | PRO | B | 336 | 0.042 | −17.221 | −34.121 | 1.00 | 66.99 | C |
| ATOM | 5 | CD | PRO | B | 336 | −0.965 | −16.916 | −35.181 | 1.00 | 66.85 | C |
| ATOM | 6 | C | PRO | B | 336 | −2.510 | −17.486 | −32.086 | 1.00 | 67.39 | C |
| ATOM | 7 | O | PRO | B | 336 | −2.762 | −18.175 | −31.097 | 1.00 | 67.44 | O |
| ATOM | 1 | N | VAL | B | 337 | −2.623 | −16.162 | −32.102 | 1.00 | 67.78 | N |
| ATOM | 2 | CA | VAL | B | 337 | −3.155 | −15.421 | −30.966 | 1.00 | 68.20 | C |
| ATOM | 3 | CB | VAL | B | 337 | −2.758 | −13.931 | −31.026 | 1.00 | 68.20 | C |
| ATOM | 4 | CG1 | VAL | B | 337 | −2.943 | −13.275 | −29.662 | 1.00 | 68.26 | C |
| ATOM | 5 | CG2 | VAL | B | 337 | −1.317 | −13.779 | −31.499 | 1.00 | 68.27 | C |
| ATOM | 6 | C | VAL | B | 337 | −4.677 | −15.540 | −30.966 | 1.00 | 68.46 | C |
| ATOM | 7 | O | VAL | B | 337 | −5.296 | −15.754 | −29.919 | 1.00 | 68.80 | O |
| ATOM | 1 | N | ILE | B | 338 | −5.269 | −15.408 | −32.150 | 1.00 | 68.70 | N |
| ATOM | 2 | CA | ILE | B | 338 | −6.710 | −15.578 | −32.322 | 1.00 | 68.88 | C |
| ATOM | 3 | CB | ILE | B | 338 | −7.127 | −15.468 | −33.806 | 1.00 | 68.80 | C |
| ATOM | 4 | CG1 | ILE | B | 338 | −6.908 | −14.045 | −34.326 | 1.00 | 68.70 | C |
| ATOM | 5 | CD1 | ILE | B | 338 | −7.913 | −13.035 | −33.805 | 1.00 | 68.75 | C |
| ATOM | 6 | CG2 | ILE | B | 338 | −8.582 | −15.865 | −33.983 | 1.00 | 68.67 | C |
| ATOM | 7 | C | ILE | B | 338 | −7.161 | −16.929 | −31.769 | 1.00 | 69.11 | C |
| ATOM | 8 | O | ILE | B | 338 | −8.075 | −16.995 | −30.944 | 1.00 | 69.27 | O |
| ATOM | 1 | N | PHE | B | 339 | −6.507 | −17.999 | −32.219 | 1.00 | 69.30 | N |
| ATOM | 2 | CA | PHE | B | 339 | −6.827 | −19.346 | −31.750 | 1.00 | 69.45 | C |
| ATOM | 3 | CB | PHE | B | 339 | −5.895 | −20.390 | −32.380 | 1.00 | 69.45 | C |
| ATOM | 4 | CG | PHE | B | 339 | −6.113 | −20.598 | −33.859 | 1.00 | 69.55 | C |
| ATOM | 5 | CD1 | PHE | B | 339 | −7.244 | −20.104 | −34.492 | 1.00 | 69.69 | C |
| ATOM | 6 | CE1 | PHE | B | 339 | −7.444 | −20.304 | −35.848 | 1.00 | 69.63 | C |
| ATOM | 7 | CZ | PHE | B | 339 | −6.519 | −21.017 | −36.584 | 1.00 | 69.58 | C |
| ATOM | 8 | CE2 | PHE | B | 339 | −5.393 | −21.524 | −35.964 | 1.00 | 69.60 | C |
| ATOM | 9 | CD2 | PHE | B | 339 | −5.195 | −21.318 | −34.610 | 1.00 | 69.56 | C |
| ATOM | 10 | C | PHE | B | 339 | −6.748 | −19.427 | −30.227 | 1.00 | 69.52 | C |
| ATOM | 11 | O | PHE | B | 339 | −7.670 | −19.923 | −29.569 | 1.00 | 69.66 | O |
| ATOM | 1 | N | ALA | B | 340 | −5.646 | −18.926 | −29.675 | 1.00 | 69.56 | N |
| ATOM | 2 | CA | ALA | B | 340 | −5.449 | −18.908 | −28.230 | 1.00 | 69.59 | C |
| ATOM | 3 | CB | ALA | B | 340 | −4.133 | −18.214 | −27.883 | 1.00 | 69.61 | C |
| ATOM | 4 | C | ALA | B | 340 | −6.619 | −18.218 | −27.532 | 1.00 | 69.61 | C |
| ATOM | 5 | O | ALA | B | 340 | −7.183 | −18.750 | −26.564 | 1.00 | 69.74 | O |
| ATOM | 1 | N | LYS | B | 341 | −6.986 | −17.035 | −28.035 | 1.00 | 69.49 | N |
| ATOM | 2 | CA | LYS | B | 341 | −8.074 | −16.263 | −27.437 | 1.00 | 69.65 | C |
| ATOM | 3 | CB | LYS | B | 341 | −8.160 | −14.861 | −28.055 | 1.00 | 69.67 | C |
| ATOM | 4 | CG | LYS | B | 341 | −6.979 | −13.952 | −27.694 | 1.00 | 69.92 | C |
| ATOM | 5 | CD | LYS | B | 341 | −7.274 | −12.477 | −27.981 | 1.00 | 69.78 | C |
| ATOM | 6 | CE | LYS | B | 341 | −6.018 | −11.612 | −27.838 | 1.00 | 69.77 | C |
| ATOM | 7 | NZ | LYS | B | 341 | −5.389 | −11.703 | −26.483 | 1.00 | 69.59 | N |
| ATOM | 8 | C | LYS | B | 341 | −9.414 | −16.993 | −27.531 | 1.00 | 69.57 | C |
| ATOM | 9 | O | LYS | B | 341 | −10.126 | −17.142 | −26.529 | 1.00 | 69.70 | O |
| ATOM | 1 | N | ILE | B | 342 | −9.748 | −17.463 | −28.731 | 1.00 | 69.50 | N |
| ATOM | 2 | CA | ILE | B | 342 | −10.994 | −18.200 | −28.933 | 1.00 | 69.48 | C |
| ATOM | 3 | CB | ILE | B | 342 | −11.209 | −18.597 | −30.405 | 1.00 | 69.39 | C |
| ATOM | 4 | CG1 | ILE | B | 342 | −11.207 | −17.354 | −31.296 | 1.00 | 69.17 | C |
| ATOM | 5 | CD1 | ILE | B | 342 | −11.610 | −17.626 | −32.724 | 1.00 | 68.85 | C |
| ATOM | 6 | CG2 | ILE | B | 342 | −12.524 | −19.337 | −30.563 | 1.00 | 69.32 | C |
| ATOM | 7 | C | ILE | B | 342 | −11.065 | −19.437 | −28.037 | 1.00 | 69.61 | C |
| ATOM | 8 | O | ILE | B | 342 | −12.132 | −19.764 | −27.519 | 1.00 | 69.60 | O |
| ATOM | 1 | N | GLN | B | 343 | −9.936 | −20.123 | −27.856 | 1.00 | 69.77 | N |
| ATOM | 2 | CA | GLN | B | 343 | −9.859 | −21.194 | −26.852 | 1.00 | 69.99 | C |
| ATOM | 3 | CB | GLN | B | 343 | −8.474 | −21.856 | −26.839 | 1.00 | 69.96 | C |
| ATOM | 4 | CG | GLN | B | 343 | −8.272 | −22.873 | −27.946 | 1.00 | 70.15 | C |
| ATOM | 5 | CD | GLN | B | 343 | −6.883 | −23.483 | −27.926 | 1.00 | 70.33 | C |
| ATOM | 6 | OE1 | GLN | B | 343 | −6.283 | −23.669 | −26.839 | 1.00 | 71.09 | O |
| ATOM | 7 | NE2 | GLN | B | 343 | −6.376 | −23.816 | −29.151 | 1.00 | 70.53 | N |
| ATOM | 8 | C | GLN | B | 343 | −10.169 | −20.640 | −25.467 | 1.00 | 70.01 | C |
| ATOM | 9 | O | GLN | B | 343 | −11.012 | −21.177 | −24.745 | 1.00 | 70.02 | O |
| ATOM | 1 | N | LEU | B | 344 | −9.477 | −19.562 | −25.105 | 1.00 | 70.01 | N |
| ATOM | 2 | CA | LEU | B | 344 | −9.665 | −18.934 | −23.801 | 1.00 | 70.13 | C |
| ATOM | 3 | CB | LEU | B | 344 | −8.882 | −17.615 | −23.707 | 1.00 | 70.20 | C |
| ATOM | 4 | CG | LEU | B | 344 | −7.366 | −17.683 | −23.472 | 1.00 | 70.29 | C |
| ATOM | 5 | CD1 | LEU | B | 344 | −6.734 | −16.303 | −23.623 | 1.00 | 70.28 | C |
| ATOM | 6 | CD2 | LEU | B | 344 | −7.043 | −18.278 | −22.105 | 1.00 | 70.32 | C |
| ATOM | 7 | C | LEU | B | 344 | −11.140 | −18.682 | −23.499 | 1.00 | 70.13 | C |
| ATOM | 8 | O | LEU | B | 344 | −11.654 | −19.142 | −22.478 | 1.00 | 70.23 | O |
| ATOM | 1 | N | GLU | B | 345 | −11.823 | −17.959 | −24.387 | 1.00 | 70.00 | N |
| ATOM | 2 | CA | GLU | B | 345 | −13.170 | −17.481 | −24.068 | 1.00 | 70.11 | C |
| ATOM | 3 | CB | GLU | B | 345 | −13.349 | −16.036 | −24.542 | 1.00 | 70.11 | C |
| ATOM | 4 | CG | GLU | B | 345 | −12.936 | −15.805 | −25.985 | 1.00 | 70.34 | C |
| ATOM | 5 | CD | GLU | B | 345 | −13.162 | −14.378 | −26.431 | 1.00 | 70.34 | C |
| ATOM | 6 | OE1 | GLU | B | 345 | −14.328 | −14.018 | −26.701 | 1.00 | 70.78 | O |
| ATOM | 7 | OE2 | GLU | B | 345 | −12.172 | −13.618 | −26.519 | 1.00 | 70.66 | O |
| ATOM | 8 | C | GLU | B | 345 | −14.330 | −18.345 | −24.570 | 1.00 | 70.06 | C |
| ATOM | 9 | O | GLU | B | 345 | −15.485 | −17.922 | −24.508 | 1.00 | 70.05 | O |
| ATOM | 1 | N | HIS | B | 346 | −14.037 | −19.545 | −25.060 | 1.00 | 70.09 | N |
| ATOM | 2 | CA | HIS | B | 346 | −15.097 | −20.438 | −25.542 | 1.00 | 70.11 | C |
| ATOM | 3 | CB | HIS | B | 346 | −15.176 | −20.431 | −27.072 | 1.00 | 70.05 | C |
| ATOM | 4 | CG | HIS | B | 346 | −16.045 | −19.349 | −27.630 | 1.00 | 69.94 | C |
| ATOM | 5 | ND1 | HIS | B | 346 | −17.419 | −19.371 | −27.525 | 1.00 | 69.85 | N |
| ATOM | 6 | CE1 | HIS | B | 346 | −17.919 | −18.296 | −28.108 | 1.00 | 70.02 | C |
| ATOM | 7 | NE2 | HIS | B | 346 | −16.919 | −17.581 | −28.594 | 1.00 | 69.91 | N |
| ATOM | 8 | CD2 | HIS | B | 346 | −15.736 | −18.218 | −28.309 | 1.00 | 70.00 | C |
| ATOM | 9 | C | HIS | B | 346 | −14.944 | −21.869 | −25.042 | 1.00 | 70.14 | C |
| ATOM | 10 | O | HIS | B | 346 | −15.736 | −22.744 | −25.397 | 1.00 | 70.16 | O |
| ATOM | 1 | N | HIS | B | 347 | −13.925 | −22.099 | −24.222 | 1.00 | 70.14 | N |
| ATOM | 2 | CA | HIS | B | 347 | −13.658 | −23.422 | −23.675 | 1.00 | 70.21 | C |
| ATOM | 3 | CB | HIS | B | 347 | −14.645 | −23.753 | −22.554 | 1.00 | 70.15 | C |
| ATOM | 4 | CG | HIS | B | 347 | −14.672 | −22.738 | −21.456 | 1.00 | 70.25 | C |
| ATOM | 5 | ND1 | HIS | B | 347 | −15.571 | −21.693 | −21.432 | 1.00 | 70.27 | N |
| ATOM | 6 | CE1 | HIS | B | 347 | −15.361 | −20.961 | −20.351 | 1.00 | 70.36 | C |
| ATOM | 7 | NE2 | HIS | B | 347 | −14.359 | −21.494 | −19.675 | 1.00 | 70.39 | N |
| ATOM | 8 | CD2 | HIS | B | 347 | −13.909 | −22.606 | −20.345 | 1.00 | 70.34 | C |
| ATOM | 9 | C | HIS | B | 347 | −13.719 | −24.505 | −24.745 | 1.00 | 70.23 | C |
| ATOM | 10 | O | HIS | B | 347 | −14.708 | −25.230 | −24.851 | 1.00 | 70.29 | O |
| ATOM | 1 | N | ILE | B | 348 | −12.665 | −24.603 | −25.547 | 1.00 | 70.25 | N |
| ATOM | 2 | CA | ILE | B | 348 | −12.514 | −25.743 | −26.442 | 1.00 | 70.37 | C |
| ATOM | 3 | CB | ILE | B | 348 | −12.829 | −25.401 | −27.922 | 1.00 | 70.40 | C |
| ATOM | 4 | CG1 | ILE | B | 348 | −12.103 | −24.131 | −28.359 | 1.00 | 70.55 | C |
| ATOM | 5 | CD1 | ILE | B | 348 | −12.926 | −22.879 | −28.183 | 1.00 | 70.97 | C |
| ATOM | 6 | CG2 | ILE | B | 348 | −14.329 | −25.226 | −28.124 | 1.00 | 70.45 | C |
| ATOM | 7 | C | ILE | B | 348 | −11.114 | −26.336 | −26.317 | 1.00 | 70.38 | C |
| ATOM | 8 | O | ILE | B | 348 | −10.144 | −25.618 | −26.065 | 1.00 | 70.43 | O |
| ATOM | 1 | N | SER | B | 349 | −11.023 | −27.653 | −26.476 | 1.00 | 70.37 | N |
| ATOM | 2 | CA | SER | B | 349 | −9.748 | −28.352 | −26.404 | 1.00 | 70.38 | C |
| ATOM | 3 | CB | SER | B | 349 | −9.971 | −29.867 | −26.344 | 1.00 | 70.40 | C |
| ATOM | 4 | OG | SER | B | 349 | −8.737 | −30.566 | −26.287 | 1.00 | 70.52 | O |
| ATOM | 5 | C | SER | B | 349 | −8.882 | −28.006 | −27.608 | 1.00 | 70.34 | C |
| ATOM | 6 | O | SER | B | 349 | −9.380 | −27.934 | −28.734 | 1.00 | 70.27 | O |
| ATOM | 1 | N | PRO | B | 350 | −7.578 | −27.779 | −27.372 | 1.00 | 70.29 | N |
| ATOM | 2 | CA | PRO | B | 350 | −6.623 | −27.530 | −28.452 | 1.00 | 70.22 | C |
| ATOM | 3 | CB | PRO | B | 350 | −5.277 | −27.495 | −27.723 | 1.00 | 70.23 | C |
| ATOM | 4 | CG | PRO | B | 350 | −5.622 | −27.067 | −26.337 | 1.00 | 70.27 | C |
| ATOM | 5 | CD | PRO | B | 350 | −6.939 | −27.722 | −26.046 | 1.00 | 70.21 | C |
| ATOM | 6 | C | PRO | B | 350 | −6.630 | −28.634 | −29.519 | 1.00 | 70.13 | C |
| ATOM | 7 | O | PRO | B | 350 | −6.142 | −28.421 | −30.630 | 1.00 | 70.22 | O |
| ATOM | 1 | N | GLY | B | 351 | −7.189 | −29.795 | −29.188 | 1.00 | 69.88 | N |
| ATOM | 2 | CA | GLY | B | 351 | −7.273 | −30.899 | −30.137 | 1.00 | 69.54 | C |
| ATOM | 3 | C | GLY | B | 351 | −8.320 | −30.680 | −31.214 | 1.00 | 69.38 | C |
| ATOM | 4 | O | GLY | B | 351 | −8.540 | −31.547 | −32.065 | 1.00 | 69.41 | O |
| ATOM | 1 | N | ASP | B | 352 | −8.968 | −29.516 | −31.183 | 1.00 | 69.15 | N |
| ATOM | 2 | CA | ASP | B | 352 | −10.039 | −29.207 | −32.128 | 1.00 | 68.90 | C |
| ATOM | 3 | CB | ASP | B | 352 | −11.248 | −28.614 | −31.401 | 1.00 | 68.94 | C |
| ATOM | 4 | CG | ASP | B | 352 | −11.970 | −29.629 | −30.543 | 1.00 | 69.10 | C |
| ATOM | 5 | OD1 | ASP | B | 352 | −13.221 | −29.587 | −30.497 | 1.00 | 69.17 | O |
| ATOM | 6 | OD2 | ASP | B | 352 | −11.288 | −30.470 | −29.914 | 1.00 | 69.78 | O |
| ATOM | 7 | C | ASP | B | 352 | −9.597 | −28.251 | −33.230 | 1.00 | 68.71 | C |
| ATOM | 8 | O | ASP | B | 352 | −10.243 | −28.157 | −34.273 | 1.00 | 68.69 | O |
| ATOM | 1 | N | PHE | B | 353 | −8.500 | −27.541 | −32.993 | 1.00 | 68.50 | N |
| ATOM | 2 | CA | PHE | B | 353 | −8.036 | −26.519 | −33.928 | 1.00 | 68.34 | C |
| ATOM | 3 | CB | PHE | B | 353 | −7.200 | −25.470 | −33.200 | 1.00 | 68.32 | C |
| ATOM | 4 | CG | PHE | B | 353 | −8.018 | −24.413 | −32.535 | 1.00 | 68.40 | C |
| ATOM | 5 | CD1 | PHE | B | 353 | −8.273 | −23.213 | −33.179 | 1.00 | 68.54 | C |
| ATOM | 6 | CE1 | PHE | B | 353 | −9.034 | −22.233 | −32.571 | 1.00 | 68.49 | C |
| ATOM | 7 | CZ | PHE | B | 353 | −9.553 | −22.448 | −31.312 | 1.00 | 68.51 | C |
| ATOM | 8 | CE2 | PHE | B | 353 | −9.311 | −23.646 | −30.663 | 1.00 | 68.68 | C |
| ATOM | 9 | CD2 | PHE | B | 353 | −8.549 | −24.620 | −31.275 | 1.00 | 68.56 | C |
| ATOM | 10 | C | PHE | B | 353 | −7.261 | −27.066 | −35.120 | 1.00 | 68.21 | C |
| ATOM | 11 | O | PHE | B | 353 | −6.497 | −28.021 | −34.987 | 1.00 | 68.28 | O |
| ATOM | 1 | N | PRO | B | 354 | −7.449 | −26.445 | −36.294 | 1.00 | 68.04 | N |
| ATOM | 2 | CA | PRO | B | 354 | −6.724 | −26.839 | −37.498 | 1.00 | 67.82 | C |
| ATOM | 3 | CB | PRO | B | 354 | −7.431 | −26.057 | −38.616 | 1.00 | 67.87 | C |
| ATOM | 4 | CG | PRO | B | 354 | −8.645 | −25.426 | −37.984 | 1.00 | 67.91 | C |
| ATOM | 5 | CD | PRO | B | 354 | −8.359 | −25.313 | −36.535 | 1.00 | 67.93 | C |
| ATOM | 6 | C | PRO | B | 354 | −5.273 | −26.392 | −37.407 | 1.00 | 67.54 | C |
| ATOM | 7 | O | PRO | B | 354 | −4.906 | −25.682 | −36.471 | 1.00 | 67.44 | O |
| ATOM | 1 | N | ASP | B | 355 | −4.456 | −26.806 | −38.371 | 1.00 | 67.25 | N |
| ATOM | 2 | CA | ASP | B | 355 | −3.091 | −26.307 | −38.464 | 1.00 | 66.95 | C |
| ATOM | 3 | CB | ASP | B | 355 | −2.306 | −27.057 | −39.542 | 1.00 | 66.98 | C |
| ATOM | 4 | CG | ASP | B | 355 | −0.873 | −26.569 | −39.667 | 1.00 | 67.19 | C |
| ATOM | 5 | OD1 | ASP | B | 355 | 0.041 | −27.421 | −39.696 | 1.00 | 67.62 | O |
| ATOM | 6 | OD2 | ASP | B | 355 | −0.655 | −25.338 | −39.735 | 1.00 | 67.10 | O |
| ATOM | 7 | C | ASP | B | 355 | −3.127 | −24.813 | −38.772 | 1.00 | 66.67 | C |
| ATOM | 8 | O | ASP | B | 355 | −3.760 | −24.383 | −39.739 | 1.00 | 66.78 | O |
| ATOM | 1 | N | CYS | B | 356 | −2.445 | −24.029 | −37.943 | 1.00 | 66.16 | N |
| ATOM | 2 | CA | CYS | B | 356 | −2.477 | −22.576 | −38.049 | 1.00 | 65.72 | C |
| ATOM | 3 | CB | CYS | B | 356 | −1.656 | −21.950 | −36.919 | 1.00 | 65.76 | C |
| ATOM | 4 | SG | CYS | B | 356 | −2.029 | −20.216 | −36.593 | 1.00 | 65.98 | S |
| ATOM | 5 | C | CYS | B | 356 | −1.970 | −22.090 | −39.405 | 1.00 | 65.43 | C |
| ATOM | 6 | O | CYS | B | 356 | −2.568 | −21.203 | −40.019 | 1.00 | 64.92 | O |
| ATOM | 1 | N | GLN | B | 357 | −0.878 | −22.689 | −39.873 | 1.00 | 65.25 | N |
| ATOM | 2 | CA | GLN | B | 357 | −0.219 | −22.247 | −41.103 | 1.00 | 65.06 | C |
| ATOM | 3 | CB | GLN | B | 357 | 1.238 | −22.723 | −41.137 | 1.00 | 65.11 | C |
| ATOM | 4 | CG | GLN | B | 357 | 2.164 | −21.874 | −40.268 | 1.00 | 65.39 | C |
| ATOM | 5 | CD | GLN | B | 357 | 3.450 | −22.588 | −39.895 | 1.00 | 65.96 | C |
| ATOM | 6 | OE1 | GLN | B | 357 | 4.496 | −21.939 | −39.705 | 1.00 | 66.00 | O |
| ATOM | 7 | NE2 | GLN | B | 357 | 3.377 | −23.933 | −39.783 | 1.00 | 66.01 | N |
| ATOM | 8 | C | GLN | B | 357 | −0.966 | −22.615 | −42.391 | 1.00 | 64.82 | C |
| ATOM | 9 | O | GLN | B | 357 | −1.003 | −21.825 | −43.342 | 1.00 | 64.50 | O |
| ATOM | 1 | N | LYS | B | 358 | −1.561 | −23.805 | −42.425 | 1.00 | 64.56 | N |
| ATOM | 2 | CA | LYS | B | 358 | −2.398 | −24.192 | −43.556 | 1.00 | 64.41 | C |
| ATOM | 3 | CB | LYS | B | 358 | −2.910 | −25.623 | −43.382 | 1.00 | 64.55 | C |
| ATOM | 4 | CG | LYS | B | 358 | −3.547 | −26.218 | −44.634 | 1.00 | 65.05 | C |
| ATOM | 5 | CD | LYS | B | 358 | −2.500 | −26.546 | −45.696 | 1.00 | 65.74 | C |
| ATOM | 6 | CE | LYS | B | 358 | −3.140 | −27.141 | −46.947 | 1.00 | 66.04 | C |
| ATOM | 7 | NZ | LYS | B | 358 | −2.121 | −27.613 | −47.935 | 1.00 | 66.17 | N |
| ATOM | 8 | C | LYS | B | 358 | −3.567 | −23.215 | −43.640 | 1.00 | 64.12 | C |
| ATOM | 9 | O | LYS | B | 358 | −3.829 | −22.600 | −44.690 | 1.00 | 63.78 | O |
| ATOM | 1 | N | MET | B | 359 | −4.252 | −23.074 | −42.509 | 1.00 | 63.91 | N |
| ATOM | 2 | CA | MET | B | 359 | −5.318 | −22.101 | −42.352 | 1.00 | 63.82 | C |
| ATOM | 3 | CB | MET | B | 359 | −5.798 | −22.083 | −40.899 | 1.00 | 63.91 | C |
| ATOM | 4 | CG | MET | B | 359 | −7.134 | −21.389 | −40.669 | 1.00 | 64.20 | C |
| ATOM | 5 | SD | MET | B | 359 | −8.547 | −22.269 | −41.375 | 1.00 | 65.02 | S |
| ATOM | 6 | CE | MET | B | 359 | −9.909 | −21.348 | −40.663 | 1.00 | 64.30 | C |
| ATOM | 7 | C | MET | B | 359 | −4.822 | −20.719 | −42.766 | 1.00 | 63.69 | C |
| ATOM | 8 | O | MET | B | 359 | −5.586 | −19.907 | −43.281 | 1.00 | 63.41 | O |
| ATOM | 1 | N | GLN | B | 360 | −3.537 | −20.459 | −42.555 | 1.00 | 63.78 | N |
| ATOM | 2 | CA | GLN | B | 360 | −2.954 | −19.186 | −42.967 | 1.00 | 63.51 | C |
| ATOM | 3 | CB | GLN | B | 360 | −1.626 | −18.937 | −42.251 | 1.00 | 63.59 | C |
| ATOM | 4 | CG | GLN | B | 360 | −1.799 | −18.365 | −40.854 | 1.00 | 63.62 | C |
| ATOM | 5 | CD | GLN | B | 360 | −0.531 | −18.412 | −40.027 | 1.00 | 63.50 | C |
| ATOM | 6 | OE1 | GLN | B | 360 | −0.315 | −17.566 | −39.161 | 1.00 | 63.52 | O |
| ATOM | 7 | NE2 | GLN | B | 360 | 0.313 | −19.403 | −40.288 | 1.00 | 63.46 | N |
| ATOM | 8 | C | GLN | B | 360 | −2.797 | −19.059 | −44.484 | 1.00 | 63.40 | C |
| ATOM | 9 | O | GLN | B | 360 | −3.046 | −17.991 | −45.049 | 1.00 | 63.16 | O |
| ATOM | 1 | N | GLU | B | 361 | −2.395 | −20.144 | −45.142 | 1.00 | 63.45 | N |
| ATOM | 2 | CA | GLU | B | 361 | −2.302 | −20.151 | −46.604 | 1.00 | 63.14 | C |
| ATOM | 3 | CB | GLU | B | 361 | −1.642 | −21.442 | −47.093 | 1.00 | 63.14 | C |
| ATOM | 4 | CG | GLU | B | 361 | −0.401 | −22.350 | −47.520 | 0.00 | 50.00 | C |
| ATOM | 5 | CD | GLU | B | 361 | −0.196 | −23.530 | −48.448 | 0.00 | 50.00 | C |
| ATOM | 6 | OE1 | GLU | B | 361 | −0.650 | −24.642 | −48.107 | 0.00 | 50.00 | O |
| ATOM | 7 | OE2 | GLU | B | 361 | 0.419 | −23.346 | −49.520 | 0.00 | 50.00 | O |
| ATOM | 8 | C | GLU | B | 361 | −3.684 | −19.978 | −47.246 | 1.00 | 63.01 | C |
| ATOM | 9 | O | GLU | B | 361 | −3.882 | −19.152 | −48.168 | 1.00 | 62.82 | O |
| ATOM | 1 | N | LEU | B | 362 | −4.648 | −20.751 | −46.745 | 1.00 | 62.93 | N |
| ATOM | 2 | CA | LEU | B | 362 | −6.011 | −20.662 | −47.266 | 1.00 | 62.55 | C |
| ATOM | 3 | CB | LEU | B | 362 | −6.861 | −21.809 | −46.719 | 1.00 | 62.51 | C |
| ATOM | 4 | CG | LEU | B | 362 | −6.284 | −23.205 | −46.967 | 1.00 | 62.22 | C |
| ATOM | 5 | CD1 | LEU | B | 362 | −7.241 | −24.272 | −46.467 | 1.00 | 61.94 | C |
| ATOM | 6 | CD2 | LEU | B | 362 | −5.971 | −23.409 | −48.443 | 1.00 | 61.87 | C |
| ATOM | 7 | C | LEU | B | 362 | −6.654 | −19.299 | −46.962 | 1.00 | 62.44 | C |
| ATOM | 8 | O | LEU | B | 362 | −7.311 | −18.678 | −47.831 | 1.00 | 62.11 | O |
| ATOM | 1 | N | LEU | B | 363 | −6.460 | −18.832 | −45.727 | 1.00 | 62.56 | N |
| ATOM | 2 | CA | LEU | B | 363 | −6.912 | −17.501 | −45.353 | 1.00 | 62.37 | C |
| ATOM | 3 | CB | LEU | B | 363 | −6.580 | −17.199 | −43.890 | 1.00 | 62.32 | C |
| ATOM | 4 | CG | LEU | B | 363 | −7.712 | −17.346 | −42.870 | 1.00 | 62.11 | C |
| ATOM | 5 | CD1 | LEU | B | 363 | −8.825 | −16.381 | −43.199 | 1.00 | 62.15 | C |
| ATOM | 6 | CD2 | LEU | B | 363 | −8.253 | −18.759 | −42.827 | 1.00 | 61.97 | C |
| ATOM | 7 | C | LEU | B | 363 | −6.259 | −16.483 | −46.274 | 1.00 | 62.38 | C |
| ATOM | 8 | O | LEU | B | 363 | −6.862 | −15.468 | −46.622 | 1.00 | 62.29 | O |
| ATOM | 1 | N | MET | B | 364 | −5.021 | −16.770 | −46.670 | 1.00 | 62.56 | N |
| ATOM | 2 | CA | MET | B | 364 | −4.321 | −15.949 | −47.654 | 1.00 | 62.46 | C |
| ATOM | 3 | CB | MET | B | 364 | −2.876 | −16.436 | −47.836 | 1.00 | 62.50 | C |
| ATOM | 4 | CG | MET | B | 364 | −1.949 | −15.444 | −48.526 | 1.00 | 62.64 | C |
| ATOM | 5 | SD | MET | B | 364 | −1.755 | −13.900 | −47.604 | 1.00 | 63.45 | S |
| ATOM | 6 | CE | MET | B | 364 | −0.474 | −13.066 | −48.552 | 1.00 | 63.02 | C |
| ATOM | 7 | C | MET | B | 364 | −5.076 | −16.000 | −48.980 | 1.00 | 62.32 | C |
| ATOM | 8 | O | MET | B | 364 | −5.079 | −15.030 | −49.741 | 1.00 | 62.24 | O |
| ATOM | 1 | N | ALA | B | 365 | −5.727 | −17.132 | −49.245 | 1.00 | 62.25 | N |
| ATOM | 2 | CA | ALA | B | 365 | −6.514 | −17.277 | −50.482 | 1.00 | 61.89 | C |
| ATOM | 3 | CB | ALA | B | 365 | −6.630 | −18.749 | −50.871 | 1.00 | 61.88 | C |
| ATOM | 4 | C | ALA | B | 365 | −7.906 | −16.615 | −50.484 | 1.00 | 61.73 | C |
| ATOM | 5 | O | ALA | B | 365 | −8.401 | −16.219 | −51.539 | 1.00 | 61.65 | O |
| ATOM | 1 | N | HIS | B | 366 | −8.539 | −16.491 | −49.318 | 1.00 | 61.58 | N |
| ATOM | 2 | CA | HIS | B | 366 | −9.928 | −15.979 | −49.260 | 1.00 | 61.07 | C |
| ATOM | 3 | CB | HIS | B | 366 | −10.678 | −16.621 | −48.097 | 1.00 | 61.07 | C |
| ATOM | 4 | CG | HIS | B | 366 | −11.201 | −17.983 | −48.405 | 1.00 | 60.77 | C |
| ATOM | 5 | ND1 | HIS | B | 366 | −10.381 | −19.026 | −48.776 | 1.00 | 60.88 | N |
| ATOM | 6 | CE1 | HIS | B | 366 | −11.113 | −20.104 | −48.989 | 1.00 | 61.03 | C |
| ATOM | 7 | NE2 | HIS | B | 366 | −12.379 | −19.796 | −48.769 | 1.00 | 61.08 | N |
| ATOM | 8 | CD2 | HIS | B | 366 | −12.461 | −18.475 | −48.402 | 1.00 | 60.66 | C |
| ATOM | 9 | C | HIS | B | 366 | −10.106 | −14.462 | −49.169 | 1.00 | 60.91 | C |
| ATOM | 10 | O | HIS | B | 366 | −9.159 | −13.732 | −48.884 | 1.00 | 60.95 | O |
| ATOM | 1 | N | ASP | B | 367 | −11.337 | −14.001 | −49.407 | 1.00 | 60.67 | N |
| ATOM | 2 | CA | ASP | B | 367 | −11.709 | −12.600 | −49.181 | 1.00 | 60.54 | C |
| ATOM | 3 | CB | ASP | B | 367 | −12.589 | −12.052 | −50.307 | 1.00 | 60.59 | C |
| ATOM | 4 | CG | ASP | B | 367 | −13.051 | −10.615 | −50.044 | 1.00 | 60.69 | C |
| ATOM | 5 | OD1 | ASP | B | 367 | −13.641 | −9.993 | −50.956 | 1.00 | 60.49 | O |
| ATOM | 6 | OD2 | ASP | B | 367 | −12.824 | −10.103 | −48.925 | 1.00 | 60.90 | O |
| ATOM | 7 | C | ASP | B | 367 | −12.447 | −12.442 | −47.863 | 1.00 | 60.51 | C |
| ATOM | 8 | O | ASP | B | 367 | −13.446 | −13.117 | −47.610 | 1.00 | 60.68 | O |
| ATOM | 1 | N | PHE | B | 368 | −11.968 | −11.519 | −47.038 | 1.00 | 60.30 | N |
| ATOM | 2 | CA | PHE | B | 368 | −12.480 | −11.376 | −45.689 | 1.00 | 60.01 | C |
| ATOM | 3 | CB | PHE | B | 368 | −11.456 | −10.666 | −44.807 | 1.00 | 60.10 | C |
| ATOM | 4 | CG | PHE | B | 368 | −10.346 | −11.562 | −44.336 | 1.00 | 60.32 | C |
| ATOM | 5 | CD1 | PHE | B | 368 | −9.815 | −12.532 | −45.176 | 1.00 | 60.54 | C |
| ATOM | 6 | CE1 | PHE | B | 368 | −8.795 | −13.358 | −44.750 | 1.00 | 60.45 | C |
| ATOM | 7 | CZ | PHE | B | 368 | −8.284 | −13.217 | −43.475 | 1.00 | 60.53 | C |
| ATOM | 8 | CE2 | PHE | B | 368 | −8.799 | −12.251 | −42.627 | 1.00 | 60.66 | C |
| ATOM | 9 | CD2 | PHE | B | 368 | −9.823 | −11.430 | −43.060 | 1.00 | 60.62 | C |
| ATOM | 10 | C | PHE | B | 368 | −13.824 | −10.673 | −45.645 | 1.00 | 59.80 | C |
| ATOM | 11 | O | PHE | B | 368 | −14.704 | −11.067 | −44.885 | 1.00 | 59.86 | O |
| ATOM | 1 | N | THR | B | 369 | −13.986 | −9.641 | −46.466 | 1.00 | 59.53 | N |
| ATOM | 2 | CA | THR | B | 369 | −15.217 | −8.854 | −46.451 | 1.00 | 59.25 | C |
| ATOM | 3 | CB | THR | B | 369 | −15.113 | −7.593 | −47.340 | 1.00 | 59.18 | C |
| ATOM | 4 | OG1 | THR | B | 369 | −14.994 | −7.974 | −48.716 | 1.00 | 59.25 | O |
| ATOM | 5 | CG2 | THR | B | 369 | −13.907 | −6.761 | −46.940 | 1.00 | 59.10 | C |
| ATOM | 6 | C | THR | B | 369 | −16.427 | −9.691 | −46.858 | 1.00 | 59.10 | C |
| ATOM | 7 | O | THR | B | 369 | −17.568 | −9.311 | −46.603 | 1.00 | 58.97 | O |
| ATOM | 1 | N | LYS | B | 370 | −16.170 | −10.840 | −47.476 | 1.00 | 59.01 | N |
| ATOM | 2 | CA | LYS | B | 370 | −17.240 | −11.725 | −47.923 | 1.00 | 58.97 | C |
| ATOM | 3 | CB | LYS | B | 370 | −16.818 | −12.482 | −49.184 | 1.00 | 59.01 | C |
| ATOM | 4 | CG | LYS | B | 370 | −16.359 | −11.596 | −50.333 | 1.00 | 59.07 | C |
| ATOM | 5 | CD | LYS | B | 370 | −17.508 | −11.175 | −51.234 | 1.00 | 59.12 | C |
| ATOM | 6 | CE | LYS | B | 370 | −16.990 | −10.481 | −52.491 | 1.00 | 59.61 | C |
| ATOM | 7 | NZ | LYS | B | 370 | −15.988 | −11.309 | −53.245 | 1.00 | 59.98 | N |
| ATOM | 8 | C | LYS | B | 370 | −17.652 | −12.715 | −46.836 | 1.00 | 58.95 | C |
| ATOM | 9 | O | LYS | B | 370 | −18.735 | −13.292 | −46.901 | 1.00 | 58.95 | O |
| ATOM | 1 | N | PHE | B | 371 | −16.785 | −12.914 | −45.844 | 1.00 | 58.98 | N |
| ATOM | 2 | CA | PHE | B | 371 | −17.095 | −13.790 | −44.713 | 1.00 | 59.03 | C |
| ATOM | 3 | CB | PHE | B | 371 | −15.974 | −13.759 | −43.669 | 1.00 | 58.86 | C |
| ATOM | 4 | CG | PHE | B | 371 | −14.716 | −14.459 | −44.098 | 1.00 | 58.79 | C |
| ATOM | 5 | CD1 | PHE | B | 371 | −13.479 | −14.003 | −43.673 | 1.00 | 58.52 | C |
| ATOM | 6 | CE1 | PHE | B | 371 | −12.318 | −14.644 | −44.060 | 1.00 | 58.30 | C |
| ATOM | 7 | CZ | PHE | B | 371 | −12.383 | −15.752 | −44.883 | 1.00 | 58.32 | C |
| ATOM | 8 | CE2 | PHE | B | 371 | −13.606 | −16.218 | −45.314 | 1.00 | 58.37 | C |
| ATOM | 9 | CD2 | PHE | B | 371 | −14.766 | −15.574 | −44.922 | 1.00 | 58.75 | C |
| ATOM | 10 | C | PHE | B | 371 | −18.402 | −13.386 | −44.043 | 1.00 | 59.21 | C |
| ATOM | 11 | O | PHE | B | 371 | −18.791 | −12.223 | −44.079 | 1.00 | 59.25 | O |
| ATOM | 1 | N | HIS | B | 372 | −19.079 | −14.346 | −43.427 | 1.00 | 59.50 | N |
| ATOM | 2 | CA | HIS | B | 372 | −20.289 | −14.040 | −42.677 | 1.00 | 59.80 | C |
| ATOM | 3 | CB | HIS | B | 372 | −21.298 | −15.180 | −42.790 | 1.00 | 59.85 | C |
| ATOM | 4 | CG | HIS | B | 372 | −21.963 | −15.261 | −44.127 | 1.00 | 59.95 | C |
| ATOM | 5 | ND1 | HIS | B | 372 | −22.899 | −14.342 | −44.548 | 1.00 | 59.92 | N |
| ATOM | 6 | CE1 | HIS | B | 372 | −23.314 | −14.663 | −45.760 | 1.00 | 60.28 | C |
| ATOM | 7 | NE2 | HIS | B | 372 | −22.678 | −15.756 | −46.142 | 1.00 | 60.47 | N |
| ATOM | 8 | CD2 | HIS | B | 372 | −21.827 | −16.150 | −45.139 | 1.00 | 60.21 | C |
| ATOM | 9 | C | HIS | B | 372 | −19.968 | −13.754 | −41.215 | 1.00 | 59.98 | C |
| ATOM | 10 | O | HIS | B | 372 | −18.885 | −14.092 | −40.734 | 1.00 | 60.08 | O |
| ATOM | 1 | N | SER | B | 373 | −20.913 | −13.130 | −40.513 | 1.00 | 60.16 | N |
| ATOM | 2 | CA | SER | B | 373 | −20.731 | −12.788 | −39.100 | 1.00 | 60.24 | C |
| ATOM | 3 | CB | SER | B | 373 | −21.512 | −11.521 | −38.748 | 1.00 | 60.28 | C |
| ATOM | 4 | OG | SER | B | 373 | −21.188 | −10.461 | −39.631 | 1.00 | 60.74 | O |
| ATOM | 5 | C | SER | B | 373 | −21.164 | −13.918 | −38.178 | 1.00 | 60.17 | C |
| ATOM | 6 | O | SER | B | 373 | −21.854 | −14.847 | −38.592 | 1.00 | 60.00 | O |
| ATOM | 1 | N | LEU | B | 374 | −20.759 | −13.828 | −36.919 | 1.00 | 60.35 | N |
| ATOM | 2 | CA | LEU | B | 374 | −21.161 | −14.813 | −35.928 | 1.00 | 60.62 | C |
| ATOM | 3 | CB | LEU | B | 374 | −20.625 | −14.424 | −34.550 | 1.00 | 60.56 | C |
| ATOM | 4 | CG | LEU | B | 374 | −20.572 | −15.531 | −33.499 | 1.00 | 60.72 | C |
| ATOM | 5 | CD1 | LEU | B | 374 | −19.481 | −15.247 | −32.474 | 1.00 | 61.21 | C |
| ATOM | 6 | CD2 | LEU | B | 374 | −21.920 | −15.707 | −32.823 | 1.00 | 60.74 | C |
| ATOM | 7 | C | LEU | B | 374 | −22.684 | −14.936 | −35.911 | 1.00 | 60.76 | C |
| ATOM | 8 | O | LEU | B | 374 | −23.394 | −13.933 | −35.966 | 1.00 | 60.93 | O |
| ATOM | 1 | N | LYS | B | 375 | −23.182 | −16.167 | −35.853 | 1.00 | 60.82 | N |
| ATOM | 2 | CA | LYS | B | 375 | −24.620 | −16.420 | −35.868 | 1.00 | 60.84 | C |
| ATOM | 3 | CB | LYS | B | 375 | −25.035 | −17.002 | −37.219 | 1.00 | 60.91 | C |
| ATOM | 4 | CG | LYS | B | 375 | −24.869 | −16.044 | −38.385 | 1.00 | 61.48 | C |
| ATOM | 5 | CD | LYS | B | 375 | −26.197 | −15.434 | −38.799 | 1.00 | 62.71 | C |
| ATOM | 6 | CE | LYS | B | 375 | −27.053 | −16.457 | −39.546 | 1.00 | 63.51 | C |
| ATOM | 7 | NZ | LYS | B | 375 | −28.354 | −15.899 | −40.015 | 1.00 | 63.82 | N |
| ATOM | 8 | C | LYS | B | 375 | −24.991 | −17.381 | −34.745 | 1.00 | 60.68 | C |
| ATOM | 9 | O | LYS | B | 375 | −24.979 | −18.595 | −34.937 | 1.00 | 60.73 | O |
| ATOM | 1 | N | PRO | B | 376 | −25.338 | −16.837 | −33.570 | 1.00 | 60.57 | N |
| ATOM | 2 | CA | PRO | B | 376 | −25.518 | −17.632 | −32.357 | 1.00 | 60.57 | C |
| ATOM | 3 | CB | PRO | B | 376 | −26.021 | −16.607 | −31.338 | 1.00 | 60.60 | C |
| ATOM | 4 | CG | PRO | B | 376 | −25.546 | −15.295 | −31.849 | 1.00 | 60.61 | C |
| ATOM | 5 | CD | PRO | B | 376 | −25.597 | −15.407 | −33.339 | 1.00 | 60.55 | C |
| ATOM | 6 | C | PRO | B | 376 | −26.542 | −18.746 | −32.530 | 1.00 | 60.59 | C |
| ATOM | 7 | O | PRO | B | 376 | −26.407 | −19.809 | −31.927 | 1.00 | 60.69 | O |
| ATOM | 1 | N | LYS | B | 377 | −27.550 | −18.495 | −33.358 | 1.00 | 60.59 | N |
| ATOM | 2 | CA | LYS | B | 377 | −28.631 | −19.446 | −33.598 | 1.00 | 60.71 | C |
| ATOM | 3 | CB | LYS | B | 377 | −29.629 | −18.839 | −34.588 | 1.00 | 60.74 | C |
| ATOM | 4 | CG | LYS | B | 377 | −29.017 | −17.771 | −35.499 | 1.00 | 61.05 | C |
| ATOM | 5 | CD | LYS | B | 377 | −29.957 | −17.378 | −36.637 | 1.00 | 61.19 | C |
| ATOM | 6 | CE | LYS | B | 377 | −30.073 | −18.489 | −37.682 | 1.00 | 61.82 | C |
| ATOM | 7 | NZ | LYS | B | 377 | −31.028 | −18.146 | −38.781 | 1.00 | 62.06 | N |
| ATOM | 8 | C | LYS | B | 377 | −28.154 | −20.821 | −34.085 | 1.00 | 60.59 | C |
| ATOM | 9 | O | LYS | B | 377 | −28.432 | −21.849 | −33.454 | 1.00 | 60.69 | O |
| ATOM | 1 | N | LEU | B | 378 | −27.435 | −20.834 | −35.205 | 1.00 | 60.51 | N |
| ATOM | 2 | CA | LEU | B | 378 | −26.950 | −22.076 | −35.806 | 1.00 | 60.41 | C |
| ATOM | 3 | CB | LEU | B | 378 | −26.243 | −21.783 | −37.126 | 1.00 | 60.35 | C |
| ATOM | 4 | CG | LEU | B | 378 | −27.021 | −20.939 | −38.140 | 1.00 | 60.12 | C |
| ATOM | 5 | CD1 | LEU | B | 378 | −26.117 | −20.552 | −39.295 | 1.00 | 59.86 | C |
| ATOM | 6 | CD2 | LEU | B | 378 | −28.267 | −21.663 | −38.641 | 1.00 | 59.70 | C |
| ATOM | 7 | C | LEU | B | 378 | −26.007 | −22.807 | −34.861 | 1.00 | 60.42 | C |
| ATOM | 8 | O | LEU | B | 378 | −26.128 | −24.019 | −34.645 | 1.00 | 60.64 | O |
| ATOM | 1 | N | LEU | B | 379 | −25.064 | −22.056 | −34.305 | 1.00 | 60.36 | N |
| ATOM | 2 | CA | LEU | B | 379 | −24.186 | −22.568 | −33.266 | 1.00 | 60.34 | C |
| ATOM | 3 | CB | LEU | B | 379 | −23.368 | −21.425 | −32.669 | 1.00 | 60.18 | C |
| ATOM | 4 | CG | LEU | B | 379 | −22.157 | −21.020 | −33.511 | 1.00 | 59.81 | C |
| ATOM | 5 | CD1 | LEU | B | 379 | −22.024 | −19.515 | −33.633 | 1.00 | 59.39 | C |
| ATOM | 6 | CD2 | LEU | B | 379 | −20.894 | −21.632 | −32.931 | 1.00 | 59.83 | C |
| ATOM | 7 | C | LEU | B | 379 | −24.993 | −23.284 | −32.188 | 1.00 | 60.50 | C |
| ATOM | 8 | O | LEU | B | 379 | −24.778 | −24.470 | −31.922 | 1.00 | 60.81 | O |
| ATOM | 1 | N | GLU | B | 380 | −25.932 | −22.567 | −31.579 | 1.00 | 60.65 | N |
| ATOM | 2 | CA | GLU | B | 380 | −26.808 | −23.167 | −30.580 | 1.00 | 60.83 | C |
| ATOM | 3 | CB | GLU | B | 380 | −27.938 | −22.214 | −30.192 | 1.00 | 60.85 | C |
| ATOM | 4 | CG | GLU | B | 380 | −27.618 | −21.304 | −29.025 | 1.00 | 60.88 | C |
| ATOM | 5 | CD | GLU | B | 380 | −28.855 | −20.937 | −28.235 | 1.00 | 61.03 | C |
| ATOM | 6 | OE1 | GLU | B | 380 | −28.781 | −20.931 | −26.991 | 1.00 | 61.38 | O |
| ATOM | 7 | OE2 | GLU | B | 380 | −29.907 | −20.673 | −28.854 | 1.00 | 61.20 | O |
| ATOM | 8 | C | GLU | B | 380 | −27.396 | −24.470 | −31.097 | 1.00 | 60.92 | C |
| ATOM | 9 | O | GLU | B | 380 | −27.372 | −25.499 | −30.402 | 1.00 | 61.26 | O |
| ATOM | 1 | N | ALA | B | 381 | −27.924 | −24.420 | −32.319 | 1.00 | 60.89 | N |
| ATOM | 2 | CA | ALA | B | 381 | −28.484 | −25.606 | −32.952 | 1.00 | 61.02 | C |
| ATOM | 3 | CB | ALA | B | 381 | −28.820 | −25.322 | −34.403 | 1.00 | 60.97 | C |
| ATOM | 4 | C | ALA | B | 381 | −27.491 | −26.754 | −32.847 | 1.00 | 61.14 | C |
| ATOM | 5 | O | ALA | B | 381 | −27.844 | −27.872 | −32.458 | 1.00 | 61.43 | O |
| ATOM | 1 | N | LEU | B | 382 | −26.241 | −26.460 | −33.182 | 1.00 | 61.13 | N |
| ATOM | 2 | CA | LEU | B | 382 | −25.166 | −27.438 | −33.083 | 1.00 | 61.34 | C |
| ATOM | 3 | CB | LEU | B | 382 | −23.865 | −26.808 | −33.592 | 1.00 | 61.35 | C |
| ATOM | 4 | CG | LEU | B | 382 | −22.699 | −27.671 | −34.081 | 1.00 | 61.48 | C |
| ATOM | 5 | CD1 | LEU | B | 382 | −21.636 | −27.835 | −32.998 | 1.00 | 61.27 | C |
| ATOM | 6 | CD2 | LEU | B | 382 | −23.194 | −29.019 | −34.602 | 1.00 | 62.11 | C |
| ATOM | 7 | C | LEU | B | 382 | −25.006 | −27.964 | −31.647 | 1.00 | 61.46 | C |
| ATOM | 8 | O | LEU | B | 382 | −25.236 | −29.157 | −31.374 | 1.00 | 61.78 | O |
| ATOM | 1 | N | ASP | B | 383 | −24.627 | −27.070 | −30.733 | 1.00 | 61.44 | N |
| ATOM | 2 | CA | ASP | B | 383 | −24.367 | −27.453 | −29.340 | 1.00 | 61.75 | C |
| ATOM | 3 | CB | ASP | B | 383 | −24.243 | −26.209 | −28.452 | 1.00 | 61.77 | C |
| ATOM | 4 | CG | ASP | B | 383 | −23.278 | −25.172 | −29.015 | 1.00 | 61.90 | C |
| ATOM | 5 | OD1 | ASP | B | 383 | −22.998 | −25.208 | −30.229 | 1.00 | 62.19 | O |
| ATOM | 6 | OD2 | ASP | B | 383 | −22.802 | −24.311 | −28.243 | 1.00 | 62.06 | O |
| ATOM | 7 | C | ASP | B | 383 | −25.474 | −28.358 | −28.809 | 1.00 | 61.90 | C |
| ATOM | 8 | O | ASP | B | 383 | −25.222 | −29.471 | −28.294 | 1.00 | 62.21 | O |
| ATOM | 1 | N | ASP | B | 384 | −26.704 | −27.870 | −28.949 | 1.00 | 61.93 | N |
| ATOM | 2 | CA | ASP | B | 384 | −27.877 | −28.633 | −28.554 | 1.00 | 62.20 | C |
| ATOM | 3 | CB | ASP | B | 384 | −29.159 | −27.857 | −28.871 | 1.00 | 62.22 | C |
| ATOM | 4 | CG | ASP | B | 384 | −30.405 | −28.552 | −28.356 | 1.00 | 62.42 | C |
| ATOM | 5 | OD1 | ASP | B | 384 | −30.668 | −28.477 | −27.136 | 1.00 | 62.53 | O |
| ATOM | 6 | OD2 | ASP | B | 384 | −31.125 | −29.168 | −29.172 | 1.00 | 62.70 | O |
| ATOM | 7 | C | ASP | B | 384 | −27.887 | −29.985 | −29.262 | 1.00 | 62.27 | C |
| ATOM | 8 | O | ASP | B | 384 | −28.130 | −31.022 | −28.622 | 1.00 | 62.50 | O |
| ATOM | 1 | N | MET | B | 385 | −27.609 | −29.969 | −30.573 | 1.00 | 62.20 | N |
| ATOM | 2 | CA | MET | B | 385 | −27.594 | −31.206 | −31.352 | 1.00 | 62.37 | C |
| ATOM | 3 | CB | MET | B | 385 | −27.031 | −31.035 | −32.769 | 1.00 | 62.38 | C |
| ATOM | 4 | CG | MET | B | 385 | −26.951 | −32.383 | −33.532 | 1.00 | 62.39 | C |
| ATOM | 5 | SD | MET | B | 385 | −25.635 | −32.605 | −34.766 | 1.00 | 62.11 | S |
| ATOM | 6 | CE | MET | B | 385 | −24.227 | −33.021 | −33.730 | 1.00 | 61.97 | C |
| ATOM | 7 | C | MET | B | 385 | −26.744 | −32.216 | −30.639 | 1.00 | 62.48 | C |
| ATOM | 8 | O | MET | B | 385 | −27.260 | −33.215 | −30.115 | 1.00 | 62.86 | O |
| ATOM | 1 | N | LEU | B | 386 | −25.437 | −31.958 | −30.611 | 1.00 | 62.43 | N |
| ATOM | 2 | CA | LEU | B | 386 | −24.558 | −32.982 | −30.070 | 1.00 | 62.76 | C |
| ATOM | 3 | CB | LEU | B | 386 | −23.056 | −32.642 | −30.160 | 1.00 | 62.76 | C |
| ATOM | 4 | CG | LEU | B | 386 | −22.440 | −31.254 | −29.955 | 1.00 | 62.69 | C |
| ATOM | 5 | CD1 | LEU | B | 386 | −20.974 | −31.394 | −29.566 | 1.00 | 62.40 | C |
| ATOM | 6 | CD2 | LEU | B | 386 | −22.572 | −30.425 | −31.218 | 1.00 | 62.82 | C |
| ATOM | 7 | C | LEU | B | 386 | −25.015 | −33.334 | −28.666 | 1.00 | 62.86 | C |
| ATOM | 8 | O | LEU | B | 386 | −25.436 | −34.480 | −28.422 | 1.00 | 63.18 | O |
| ATOM | 1 | N | ALA | B | 387 | −25.006 | −32.335 | −27.779 | 1.00 | 62.84 | N |
| ATOM | 2 | CA | ALA | B | 387 | −25.437 | −32.545 | −26.396 | 1.00 | 63.11 | C |
| ATOM | 3 | CB | ALA | B | 387 | −25.713 | −31.207 | −25.715 | 1.00 | 62.92 | C |
| ATOM | 4 | C | ALA | B | 387 | −26.663 | −33.465 | −26.298 | 1.00 | 63.24 | C |
| ATOM | 5 | O | ALA | B | 387 | −26.542 | −34.694 | −26.107 | 1.00 | 63.61 | O |
| ATOM | 1 | N | GLN | B | 388 | −27.843 | −32.877 | −26.467 | 1.00 | 63.14 | N |
| ATOM | 2 | CA | GLN | B | 388 | −29.082 | −33.594 | −26.185 | 1.00 | 63.43 | C |
| ATOM | 3 | CB | GLN | B | 388 | −30.244 | −32.603 | −26.059 | 1.00 | 63.49 | C |
| ATOM | 4 | CG | GLN | B | 388 | −29.854 | −31.297 | −25.371 | 1.00 | 63.57 | C |
| ATOM | 5 | CD | GLN | B | 388 | −30.908 | −30.790 | −24.402 | 1.00 | 63.61 | C |
| ATOM | 6 | OE1 | GLN | B | 388 | −31.675 | −31.570 | −23.830 | 1.00 | 63.36 | O |
| ATOM | 7 | NE2 | GLN | B | 388 | −30.941 | −29.477 | −24.202 | 1.00 | 63.79 | N |
| ATOM | 8 | C | GLN | B | 388 | −29.406 | −34.688 | −27.207 | 1.00 | 63.52 | C |
| ATOM | 9 | O | GLN | B | 388 | −29.698 | −35.850 | −26.842 | 1.00 | 63.82 | O |
| ATOM | 1 | N | ASP | B | 389 | −29.344 | −34.323 | −28.486 | 1.00 | 63.47 | N |
| ATOM | 2 | CA | ASP | B | 389 | −29.769 | −35.242 | −29.529 | 1.00 | 63.75 | C |
| ATOM | 3 | CB | ASP | B | 389 | −29.774 | −34.549 | −30.891 | 1.00 | 63.76 | C |
| ATOM | 4 | CG | ASP | B | 389 | −30.851 | −33.484 | −30.995 | 1.00 | 64.01 | C |
| ATOM | 5 | OD1 | ASP | B | 389 | −32.035 | −33.801 | −30.746 | 1.00 | 64.41 | O |
| ATOM | 6 | OD2 | ASP | B | 389 | −30.517 | −32.328 | −31.329 | 1.00 | 64.29 | O |
| ATOM | 7 | C | ASP | B | 389 | −28.895 | −36.491 | −29.545 | 1.00 | 63.83 | C |
| ATOM | 8 | O | ASP | B | 389 | −29.409 | −37.633 | −29.769 | 1.00 | 64.19 | O |
| ATOM | 1 | N | ILE | B | 390 | −27.582 | −36.290 | −29.283 | 1.00 | 63.79 | N |
| ATOM | 2 | CA | ILE | B | 390 | −26.702 | −37.449 | −29.319 | 1.00 | 64.00 | C |
| ATOM | 3 | CB | ILE | B | 390 | −25.257 | −37.051 | −29.645 | 1.00 | 63.95 | C |
| ATOM | 4 | CG1 | ILE | B | 390 | −25.170 | −36.707 | −31.135 | 1.00 | 63.71 | C |
| ATOM | 5 | CD1 | ILE | B | 390 | −23.857 | −36.139 | −31.565 | 1.00 | 63.77 | C |
| ATOM | 6 | CG2 | ILE | B | 390 | −24.289 | −38.171 | −29.283 | 1.00 | 63.97 | C |
| ATOM | 7 | C | ILE | B | 390 | −26.849 | −38.215 | −28.008 | 1.00 | 64.14 | C |
| ATOM | 8 | O | ILE | B | 390 | −26.847 | −39.465 | −27.987 | 1.00 | 64.58 | O |
| ATOM | 1 | N | ALA | B | 391 | −27.030 | −37.458 | −26.925 | 1.00 | 64.14 | N |
| ATOM | 2 | CA | ALA | B | 391 | −27.407 | −38.063 | −25.658 | 1.00 | 64.26 | C |
| ATOM | 3 | CB | ALA | B | 391 | −27.864 | −37.001 | −24.673 | 1.00 | 64.25 | C |
| ATOM | 4 | C | ALA | B | 391 | −28.498 | −39.120 | −25.865 | 1.00 | 64.38 | C |
| ATOM | 5 | O | ALA | B | 391 | −28.328 | −40.265 | −25.456 | 1.00 | 64.34 | O |
| ATOM | 1 | N | LYS | B | 392 | −29.600 | −38.750 | −26.522 | 1.00 | 64.51 | N |
| ATOM | 2 | CA | LYS | B | 392 | −30.752 | −39.681 | −26.672 | 1.00 | 65.03 | C |
| ATOM | 3 | CB | LYS | B | 392 | −32.041 | −38.900 | −27.039 | 1.00 | 65.00 | C |
| ATOM | 4 | CG | LYS | B | 392 | −33.202 | −39.822 | −27.422 | 1.00 | 65.42 | C |
| ATOM | 5 | CD | LYS | B | 392 | −34.304 | −39.084 | −28.196 | 1.00 | 65.51 | C |
| ATOM | 6 | CE | LYS | B | 392 | −33.876 | −38.797 | −29.657 | 1.00 | 66.25 | C |
| ATOM | 7 | NZ | LYS | B | 392 | −34.960 | −38.108 | −30.448 | 1.00 | 66.63 | N |
| ATOM | 8 | C | LYS | B | 392 | −30.564 | −40.824 | −27.719 | 1.00 | 65.09 | C |
| ATOM | 9 | O | LYS | B | 392 | −30.922 | −42.067 | −27.733 | 1.00 | 65.53 | O |
| ATOM | 1 | N | LEU | B | 393 | −29.500 | −40.372 | −28.661 | 1.00 | 65.09 | N |
| ATOM | 2 | CA | LEU | B | 393 | −29.270 | −41.413 | −29.680 | 1.00 | 65.41 | C |
| ATOM | 3 | CB | LEU | B | 393 | −28.567 | −40.850 | −30.917 | 1.00 | 65.35 | C |
| ATOM | 4 | CG | LEU | B | 393 | −29.572 | −40.402 | −31.975 | 1.00 | 65.48 | C |
| ATOM | 5 | CD1 | LEU | B | 393 | −28.854 | −39.831 | −33.175 | 1.00 | 65.63 | C |
| ATOM | 6 | CD2 | LEU | B | 393 | −30.454 | −41.576 | −32.381 | 1.00 | 65.50 | C |
| ATOM | 7 | C | LEU | B | 393 | −28.469 | −42.559 | −29.072 | 1.00 | 65.64 | C |
| ATOM | 8 | O | LEU | B | 393 | −28.537 | −43.692 | −29.565 | 1.00 | 65.90 | O |
| ATOM | 1 | N | MET | B | 394 | −27.727 | −42.276 | −27.994 | 1.00 | 65.68 | N |
| ATOM | 2 | CA | MET | B | 394 | −26.953 | −43.350 | −27.351 | 1.00 | 66.07 | C |
| ATOM | 3 | CB | MET | B | 394 | −26.160 | −42.822 | −26.147 | 1.00 | 66.11 | C |
| ATOM | 4 | CG | MET | B | 394 | −25.045 | −41.845 | −26.512 | 1.00 | 66.43 | C |
| ATOM | 5 | SD | MET | B | 394 | −23.879 | −42.505 | −27.732 | 1.00 | 67.28 | S |
| ATOM | 6 | CE | MET | B | 394 | −22.646 | −41.197 | −27.775 | 1.00 | 66.63 | C |
| ATOM | 7 | C | MET | B | 394 | −27.834 | −44.552 | −26.951 | 1.00 | 66.21 | C |
| ATOM | 8 | O | MET | B | 394 | −27.625 | −45.678 | −27.434 | 1.00 | 66.49 | O |
| ATOM | 1 | N | PRO | B | 395 | −28.824 | −44.319 | −26.063 | 1.00 | 66.35 | N |
| ATOM | 2 | CA | PRO | B | 395 | −29.721 | −45.400 | −25.637 | 1.00 | 66.36 | C |
| ATOM | 3 | CB | PRO | B | 395 | −30.859 | −44.656 | −24.933 | 1.00 | 66.30 | C |
| ATOM | 4 | CG | PRO | B | 395 | −30.208 | −43.450 | −24.378 | 1.00 | 66.32 | C |
| ATOM | 5 | CD | PRO | B | 395 | −29.137 | −43.024 | −25.387 | 1.00 | 66.42 | C |
| ATOM | 6 | C | PRO | B | 395 | −30.288 | −46.183 | −26.818 | 1.00 | 66.38 | C |
| ATOM | 7 | O | PRO | B | 395 | −30.347 | −47.571 | −26.772 | 1.00 | 66.53 | O |
| ATOM | 1 | N | LEU | B | 396 | −30.497 | −45.303 | −28.030 | 1.00 | 66.35 | N |
| ATOM | 2 | CA | LEU | B | 396 | −31.246 | −46.083 | −29.062 | 1.00 | 66.51 | C |
| ATOM | 3 | CB | LEU | B | 396 | −31.852 | −45.178 | −30.154 | 1.00 | 66.52 | C |
| ATOM | 4 | CG | LEU | B | 396 | −32.912 | −45.758 | −31.111 | 1.00 | 66.50 | C |
| ATOM | 5 | CD1 | LEU | B | 396 | −33.315 | −44.715 | −32.155 | 1.00 | 66.78 | C |
| ATOM | 6 | CD2 | LEU | B | 396 | −32.466 | −47.051 | −31.807 | 1.00 | 66.41 | C |
| ATOM | 7 | C | LEU | B | 396 | −30.201 | −47.020 | −29.694 | 1.00 | 66.56 | C |
| ATOM | 8 | O | LEU | B | 396 | −30.470 | −48.217 | −30.094 | 1.00 | 66.72 | O |
| ATOM | 1 | N | LEU | B | 397 | −28.990 | −46.430 | −29.758 | 1.00 | 66.57 | N |
| ATOM | 2 | CA | LEU | B | 397 | −27.835 | −47.098 | −30.313 | 1.00 | 66.81 | C |
| ATOM | 3 | CB | LEU | B | 397 | −26.607 | −46.199 | −30.188 | 1.00 | 66.77 | C |
| ATOM | 4 | CG | LEU | B | 397 | −25.289 | −46.797 | −30.666 | 1.00 | 66.74 | C |
| ATOM | 5 | CD1 | LEU | B | 397 | −25.328 | −47.016 | −32.172 | 1.00 | 66.79 | C |
| ATOM | 6 | CD2 | LEU | B | 397 | −24.143 | −45.884 | −30.274 | 1.00 | 66.83 | C |
| ATOM | 7 | C | LEU | B | 397 | −27.582 | −48.420 | −29.616 | 1.00 | 66.95 | C |
| ATOM | 8 | O | LEU | B | 397 | −27.383 | −49.426 | −30.278 | 1.00 | 67.22 | O |
| ATOM | 1 | N | ARG | B | 398 | −27.592 | −48.416 | −28.284 | 1.00 | 67.02 | N |
| ATOM | 2 | CA | ARG | B | 398 | −27.336 | −49.643 | −27.514 | 1.00 | 67.30 | C |
| ATOM | 3 | CB | ARG | B | 398 | −27.350 | −49.332 | −26.015 | 1.00 | 67.36 | C |
| ATOM | 4 | CG | ARG | B | 398 | −26.308 | −48.309 | −25.582 | 1.00 | 67.98 | C |
| ATOM | 5 | CD | ARG | B | 398 | −26.819 | −47.456 | −24.424 | 1.00 | 68.97 | C |
| ATOM | 6 | NE | ARG | B | 398 | −27.294 | −48.259 | −23.300 | 1.00 | 69.67 | N |
| ATOM | 7 | CZ | ARG | B | 398 | −26.505 | −48.760 | −22.351 | 1.00 | 70.33 | C |
| ATOM | 8 | NH1 | ARG | B | 398 | −27.028 | −49.478 | −21.363 | 1.00 | 70.67 | N |
| ATOM | 9 | NH2 | ARG | B | 398 | −25.193 | −48.547 | −22.389 | 1.00 | 70.46 | N |
| ATOM | 10 | C | ARG | B | 398 | −28.366 | −50.744 | −27.814 | 1.00 | 67.28 | C |
| ATOM | 11 | O | ARG | B | 398 | −28.004 | −52.040 | −27.953 | 1.00 | 67.55 | O |
| ATOM | 1 | N | GLN | B | 399 | −29.649 | −50.229 | −28.027 | 1.00 | 67.15 | N |
| ATOM | 2 | CA | GLN | B | 399 | −30.706 | −51.190 | −28.344 | 1.00 | 67.22 | C |
| ATOM | 3 | CB | GLN | B | 399 | −32.089 | −50.529 | −28.308 | 1.00 | 67.21 | C |
| ATOM | 4 | CG | GLN | B | 399 | −33.256 | −51.484 | −28.565 | 1.00 | 67.20 | C |
| ATOM | 5 | CD | GLN | B | 399 | −33.255 | −52.683 | −27.622 | 1.00 | 67.24 | C |
| ATOM | 6 | OE1 | GLN | B | 399 | −33.322 | −53.832 | −28.070 | 1.00 | 67.09 | O |
| ATOM | 7 | NE2 | GLN | B | 399 | −33.174 | −52.421 | −26.312 | 1.00 | 67.33 | N |
| ATOM | 8 | C | GLN | B | 399 | −30.427 | −51.801 | −29.713 | 1.00 | 67.26 | C |
| ATOM | 9 | O | GLN | B | 399 | −30.729 | −52.973 | −29.958 | 1.00 | 67.20 | O |
| ATOM | 1 | N | GLU | B | 400 | −29.833 | −51.006 | −30.601 | 1.00 | 67.33 | N |
| ATOM | 2 | CA | GLU | B | 400 | −29.448 | −51.520 | −31.913 | 1.00 | 67.67 | C |
| ATOM | 3 | CB | GLU | B | 400 | −29.270 | −50.383 | −32.917 | 1.00 | 67.73 | C |
| ATOM | 4 | CG | GLU | B | 400 | −30.581 | −49.853 | −33.487 | 1.00 | 68.29 | C |
| ATOM | 5 | CD | GLU | B | 400 | −30.356 | −49.020 | −34.738 | 1.00 | 68.96 | C |
| ATOM | 6 | OE1 | GLU | B | 400 | −31.298 | −48.311 | −35.159 | 1.00 | 69.08 | O |
| ATOM | 7 | OE2 | GLU | B | 400 | −29.235 | −49.077 | −35.297 | 1.00 | 69.03 | O |
| ATOM | 8 | C | GLU | B | 400 | −28.181 | −52.374 | −31.867 | 1.00 | 67.75 | C |
| ATOM | 9 | O | GLU | B | 400 | −28.005 | −53.276 | −32.686 | 1.00 | 67.75 | O |
| ATOM | 1 | N | GLU | B | 401 | −27.302 | −52.089 | −30.909 | 1.00 | 67.83 | N |
| ATOM | 2 | CA | GLU | B | 401 | −26.035 | −52.807 | −30.795 | 1.00 | 68.15 | C |
| ATOM | 3 | CB | GLU | B | 401 | −25.015 | −52.036 | −29.938 | 1.00 | 68.25 | C |
| ATOM | 4 | CG | GLU | B | 401 | −23.580 | −52.606 | −30.002 | 1.00 | 68.55 | C |
| ATOM | 5 | CD | GLU | B | 401 | −22.501 | −51.549 | −29.779 | 1.00 | 68.79 | C |
| ATOM | 6 | OE1 | GLU | B | 401 | −22.629 | −50.435 | −30.334 | 1.00 | 68.79 | O |
| ATOM | 7 | OE2 | GLU | B | 401 | −21.517 | −51.839 | −29.059 | 1.00 | 68.49 | O |
| ATOM | 8 | C | GLU | B | 401 | −26.275 | −54.218 | −30.257 | 1.00 | 68.19 | C |
| ATOM | 9 | O | GLU | B | 401 | −25.363 | −55.103 | −30.342 | 1.00 | 68.20 | O |
| ATOM | 1 | N | LEU | B | 402 | −27.514 | −54.443 | −29.722 | 1.00 | 68.10 | N |
| ATOM | 2 | CA | LEU | B | 402 | −27.860 | −55.876 | −29.534 | 1.00 | 68.26 | C |
| ATOM | 3 | CB | LEU | B | 402 | −28.151 | −56.248 | −28.065 | 1.00 | 68.22 | C |
| ATOM | 4 | CG | LEU | B | 402 | −28.980 | −55.386 | −27.109 | 1.00 | 68.29 | C |
| ATOM | 5 | CD1 | LEU | B | 402 | −30.451 | −55.301 | −27.514 | 1.00 | 68.15 | C |
| ATOM | 6 | CD2 | LEU | B | 402 | −28.840 | −55.954 | −25.698 | 1.00 | 68.27 | C |
| ATOM | 7 | C | LEU | B | 402 | −28.980 | −56.322 | −30.497 | 1.00 | 68.35 | C |
| ATOM | 8 | O | LEU | B | 402 | −30.147 | −56.557 | −30.094 | 1.00 | 68.28 | O |
| ATOM | 1 | N | GLU | B | 403 | −28.607 | −56.428 | −31.781 | 1.00 | 68.52 | N |
| ATOM | 2 | CA | GLU | B | 403 | −29.588 | −56.755 | −32.818 | 1.00 | 68.65 | C |
| ATOM | 3 | CB | GLU | B | 403 | −29.931 | −55.521 | −33.659 | 1.00 | 68.70 | C |
| ATOM | 4 | CG | GLU | B | 403 | −30.991 | −54.639 | −33.021 | 1.00 | 68.74 | C |
| ATOM | 5 | CD | GLU | B | 403 | −32.215 | −55.426 | −32.590 | 1.00 | 68.53 | C |
| ATOM | 6 | OE1 | GLU | B | 403 | −32.201 | −56.670 | −32.722 | 1.00 | 68.11 | O |
| ATOM | 7 | OE2 | GLU | B | 403 | −33.191 | −54.802 | −32.122 | 1.00 | 68.50 | O |
| ATOM | 8 | C | GLU | B | 403 | −29.214 | −57.924 | −33.722 | 1.00 | 68.69 | C |
| ATOM | 9 | O | GLU | B | 403 | −29.749 | −59.023 | −33.563 | 1.00 | 68.78 | O |
| ATOM | 1 | N | SER | B | 404 | −28.317 | −57.699 | −34.679 | 1.00 | 68.70 | N |
| ATOM | 2 | CA | SER | B | 404 | −27.974 | −58.768 | −35.617 | 1.00 | 68.71 | C |
| ATOM | 3 | CB | SER | B | 404 | −28.740 | −58.599 | −36.939 | 1.00 | 68.72 | C |
| ATOM | 4 | OG | SER | B | 404 | −28.941 | −59.853 | −37.578 | 1.00 | 68.64 | O |
| ATOM | 5 | C | SER | B | 404 | −26.470 | −58.933 | −35.873 | 1.00 | 68.68 | C |
| ATOM | 6 | O | SER | B | 404 | −25.844 | −58.123 | −36.565 | 1.00 | 68.72 | O |
| ATOM | 1 | N | VAL | B | 405 | −25.905 | −59.999 | −35.307 | 1.00 | 68.52 | N |
| ATOM | 2 | CA | VAL | B | 405 | −24.514 | −60.373 | −35.553 | 1.00 | 68.31 | C |
| ATOM | 3 | CB | VAL | B | 405 | −24.015 | −61.401 | −34.501 | 1.00 | 68.37 | C |
| ATOM | 4 | CG1 | VAL | B | 405 | −22.517 | −61.661 | −34.657 | 1.00 | 68.42 | C |
| ATOM | 5 | CG2 | VAL | B | 405 | −24.816 | −62.702 | −34.582 | 1.00 | 68.30 | C |
| ATOM | 6 | C | VAL | B | 405 | −24.359 | −60.941 | −36.969 | 1.00 | 68.09 | C |
| ATOM | 7 | O | VAL | B | 405 | −23.242 | −61.172 | −37.443 | 1.00 | 68.12 | O |
| ATOM | 1 | N | GLU | B | 406 | −25.494 | −61.154 | −37.634 | 1.00 | 67.77 | N |
| ATOM | 2 | CA | GLU | B | 406 | −25.541 | −61.660 | −39.008 | 1.00 | 67.37 | C |
| ATOM | 3 | CB | GLU | B | 406 | −26.992 | −61.732 | −39.495 | 1.00 | 67.50 | C |
| ATOM | 4 | CG | GLU | B | 406 | −27.147 | −62.138 | −40.957 | 1.00 | 67.80 | C |
| ATOM | 5 | CD | GLU | B | 406 | −28.069 | −61.202 | −41.723 | 1.00 | 68.31 | C |
| ATOM | 6 | OE1 | GLU | B | 406 | −29.061 | −61.685 | −42.313 | 1.00 | 68.29 | O |
| ATOM | 7 | OE2 | GLU | B | 406 | −27.801 | −59.978 | −41.732 | 1.00 | 68.51 | O |
| ATOM | 8 | C | GLU | B | 406 | −24.734 | −60.777 | −39.957 | 1.00 | 66.93 | C |
| ATOM | 9 | O | GLU | B | 406 | −24.162 | −61.265 | −40.937 | 1.00 | 66.98 | O |
| ATOM | 1 | N | ALA | B | 407 | −24.697 | −59.479 | −39.659 | 1.00 | 66.23 | N |
| ATOM | 2 | CA | ALA | B | 407 | −23.943 | −58.516 | −40.459 | 1.00 | 65.47 | C |
| ATOM | 3 | CB | ALA | B | 407 | −24.221 | −57.094 | −39.976 | 1.00 | 65.49 | C |
| ATOM | 4 | C | ALA | B | 407 | −22.440 | −58.813 | −40.442 | 1.00 | 64.85 | C |
| ATOM | 5 | O | ALA | B | 407 | −21.702 | −58.369 | −41.328 | 1.00 | 64.84 | O |
| ATOM | 1 | N | GLY | B | 408 | −22.004 | −59.576 | −39.439 | 1.00 | 64.00 | N |
| ATOM | 2 | CA | GLY | B | 408 | −20.598 | −59.936 | −39.266 | 1.00 | 62.91 | C |
| ATOM | 3 | C | GLY | B | 408 | −19.933 | −60.428 | −40.535 | 1.00 | 62.16 | C |
| ATOM | 4 | O | GLY | B | 408 | −20.582 | −61.020 | −41.397 | 1.00 | 62.11 | O |
| ATOM | 1 | N | VAL | B | 409 | −18.633 | −60.180 | −40.649 | 1.00 | 61.40 | N |
| ATOM | 2 | CA | VAL | B | 409 | −17.880 | −60.595 | −41.824 | 1.00 | 60.66 | C |
| ATOM | 3 | CB | VAL | B | 409 | −16.452 | −60.013 | −41.824 | 1.00 | 60.68 | C |
| ATOM | 4 | CG1 | VAL | B | 409 | −15.845 | −60.103 | −43.213 | 1.00 | 60.53 | C |
| ATOM | 5 | CG2 | VAL | B | 409 | −16.473 | −58.573 | −41.362 | 1.00 | 60.66 | C |
| ATOM | 6 | C | VAL | B | 409 | −17.829 | −62.117 | −41.919 | 1.00 | 60.21 | C |
| ATOM | 7 | O | VAL | B | 409 | −17.427 | −62.803 | −40.974 | 1.00 | 60.00 | O |
| ATOM | 1 | N | ALA | B | 410 | −18.250 | −62.638 | −43.066 | 1.00 | 59.67 | N |
| ATOM | 2 | CA | ALA | B | 410 | −18.324 | −64.077 | −43.271 | 1.00 | 59.13 | C |
| ATOM | 3 | CB | ALA | B | 410 | −19.519 | −64.425 | −44.142 | 1.00 | 59.26 | C |
| ATOM | 4 | C | ALA | B | 410 | −17.043 | −64.616 | −43.885 | 1.00 | 58.71 | C |
| ATOM | 5 | O | ALA | B | 410 | −16.274 | −63.875 | −44.491 | 1.00 | 58.61 | O |
| ATOM | 1 | N | GLY | B | 411 | −16.838 | −65.919 | −43.723 | 1.00 | 58.33 | N |
| ATOM | 2 | CA | GLY | B | 411 | −15.635 | −66.617 | −44.174 | 1.00 | 57.74 | C |
| ATOM | 3 | C | GLY | B | 411 | −14.718 | −65.930 | −45.170 | 1.00 | 57.25 | C |
| ATOM | 4 | O | GLY | B | 411 | −15.164 | −65.273 | −46.113 | 1.00 | 57.29 | O |
| ATOM | 1 | N | GLY | B | 412 | −13.420 | −66.103 | −44.946 | 1.00 | 56.81 | N |
| ATOM | 2 | CA | GLY | B | 412 | −12.385 | −65.639 | −45.860 | 1.00 | 56.24 | C |
| ATOM | 3 | C | GLY | B | 412 | −11.093 | −66.100 | −45.229 | 1.00 | 55.88 | C |
| ATOM | 4 | O | GLY | B | 412 | −10.523 | −67.115 | −45.616 | 1.00 | 55.87 | O |
| ATOM | 1 | N | ALA | B | 413 | −10.649 | −65.349 | −44.232 | 1.00 | 55.53 | N |
| ATOM | 2 | CA | ALA | B | 413 | −9.619 | −65.806 | −43.319 | 1.00 | 55.19 | C |
| ATOM | 3 | CB | ALA | B | 413 | −8.452 | −64.844 | −43.313 | 1.00 | 55.14 | C |
| ATOM | 4 | C | ALA | B | 413 | −10.263 | −65.865 | −41.946 | 1.00 | 54.97 | C |
| ATOM | 5 | O | ALA | B | 413 | −9.607 | −66.140 | −40.945 | 1.00 | 55.00 | O |
| ATOM | 1 | N | PHE | B | 414 | −11.566 | −65.613 | −41.915 | 1.00 | 54.73 | N |
| ATOM | 2 | CA | PHE | B | 414 | −12.291 | −65.468 | −40.663 | 1.00 | 54.57 | C |
| ATOM | 3 | CB | PHE | B | 414 | −13.369 | −64.387 | −40.793 | 1.00 | 54.59 | C |
| ATOM | 4 | CG | PHE | B | 414 | −12.826 | −63.027 | −41.139 | 1.00 | 54.41 | C |
| ATOM | 5 | CD1 | PHE | B | 414 | −12.351 | −62.184 | −40.148 | 1.00 | 54.25 | C |
| ATOM | 6 | CE1 | PHE | B | 414 | −11.851 | −60.941 | −40.462 | 1.00 | 54.27 | C |
| ATOM | 7 | CZ | PHE | B | 414 | −11.822 | −60.521 | −41.778 | 1.00 | 54.38 | C |
| ATOM | 8 | CE2 | PHE | B | 414 | −12.292 | −61.350 | −42.774 | 1.00 | 54.20 | C |
| ATOM | 9 | CD2 | PHE | B | 414 | −12.791 | −62.594 | −42.454 | 1.00 | 54.24 | C |
| ATOM | 10 | C | PHE | B | 414 | −12.921 | −66.771 | −40.194 | 1.00 | 54.51 | C |
| ATOM | 11 | O | PHE | B | 414 | −13.036 | −67.005 | −38.991 | 1.00 | 54.59 | O |
| ATOM | 1 | N | GLU | B | 415 | −13.327 | −67.612 | −41.143 | 1.00 | 54.32 | N |
| ATOM | 2 | CA | GLU | B | 415 | −13.998 | −68.872 | −40.827 | 1.00 | 54.13 | C |
| ATOM | 3 | CB | GLU | B | 415 | −14.168 | −69.713 | −42.087 | 1.00 | 54.25 | C |
| ATOM | 4 | CG | GLU | B | 415 | −12.858 | −70.145 | −42.699 | 1.00 | 55.37 | C |
| ATOM | 5 | CD | GLU | B | 415 | −12.959 | −70.379 | −44.192 | 1.00 | 57.16 | C |
| ATOM | 6 | OE1 | GLU | B | 415 | −11.987 | −70.912 | −44.778 | 1.00 | 57.92 | O |
| ATOM | 7 | OE2 | GLU | B | 415 | −14.007 | −70.025 | −44.779 | 1.00 | 57.82 | O |
| ATOM | 8 | C | GLU | B | 415 | −13.258 | −69.664 | −39.750 | 1.00 | 53.71 | C |
| ATOM | 9 | O | GLU | B | 415 | −12.027 | −69.672 | −39.704 | 1.00 | 53.58 | O |
| ATOM | 1 | N | GLY | B | 416 | −14.023 | −70.325 | −38.886 | 1.00 | 53.38 | N |
| ATOM | 2 | CA | GLY | B | 416 | −13.466 | −70.999 | −37.718 | 1.00 | 52.98 | C |
| ATOM | 3 | C | GLY | B | 416 | −12.802 | −72.320 | −38.039 | 1.00 | 52.72 | C |
| ATOM | 4 | O | GLY | B | 416 | −11.853 | −72.729 | −37.367 | 1.00 | 52.60 | O |
| ATOM | 1 | N | THR | B | 417 | −13.305 | −72.985 | −39.075 | 1.00 | 52.54 | N |
| ATOM | 2 | CA | THR | B | 417 | −12.774 | −74.275 | −39.501 | 1.00 | 52.37 | C |
| ATOM | 3 | CB | THR | B | 417 | −13.594 | −74.865 | −40.664 | 1.00 | 52.27 | C |
| ATOM | 4 | OG1 | THR | B | 417 | −13.938 | −73.824 | −41.584 | 1.00 | 51.98 | O |
| ATOM | 5 | CG2 | THR | B | 417 | −14.869 | −75.497 | −40.147 | 1.00 | 52.29 | C |
| ATOM | 6 | C | THR | B | 417 | −11.303 | −74.199 | −39.906 | 1.00 | 52.41 | C |
| ATOM | 7 | O | THR | B | 417 | −10.666 | −75.226 | −40.137 | 1.00 | 52.44 | O |
| ATOM | 1 | N | ARG | B | 418 | −10.769 | −72.982 | −39.984 | 1.00 | 52.48 | N |
| ATOM | 2 | CA | ARG | B | 418 | −9.376 | −72.768 | −40.365 | 1.00 | 52.60 | C |
| ATOM | 3 | CB | ARG | B | 418 | −9.159 | −71.335 | −40.852 | 1.00 | 52.61 | C |
| ATOM | 4 | CG | ARG | B | 418 | −9.600 | −71.097 | −42.286 | 1.00 | 53.27 | C |
| ATOM | 5 | CD | ARG | B | 418 | −9.177 | −69.722 | −42.800 | 1.00 | 54.81 | C |
| ATOM | 6 | NE | ARG | B | 418 | −7.737 | −69.623 | −43.033 | 1.00 | 55.96 | N |
| ATOM | 7 | CZ | ARG | B | 418 | −6.871 | −69.084 | −42.177 | 1.00 | 56.80 | C |
| ATOM | 8 | NH1 | ARG | B | 418 | −5.577 | −69.038 | −42.478 | 1.00 | 57.11 | N |
| ATOM | 9 | NH2 | ARG | B | 418 | −7.295 | −68.589 | −41.019 | 1.00 | 56.93 | N |
| ATOM | 10 | C | ARG | B | 418 | −8.416 | −73.077 | −39.226 | 1.00 | 52.65 | C |
| ATOM | 11 | O | ARG | B | 418 | −7.222 | −73.271 | −39.444 | 1.00 | 52.59 | O |
| ATOM | 1 | N | MET | B | 419 | −8.943 | −73.129 | −38.010 | 1.00 | 52.86 | N |
| ATOM | 2 | CA | MET | B | 419 | −8.113 | −73.399 | −36.846 | 1.00 | 53.06 | C |
| ATOM | 3 | CB | MET | B | 419 | −8.676 | −72.685 | −35.610 | 1.00 | 53.29 | C |
| ATOM | 4 | CG | MET | B | 419 | −7.647 | −72.383 | −34.513 | 1.00 | 54.17 | C |
| ATOM | 5 | SD | MET | B | 419 | −6.116 | −71.599 | −35.095 | 1.00 | 55.92 | S |
| ATOM | 6 | CE | MET | B | 419 | −4.994 | −72.997 | −35.162 | 1.00 | 55.37 | C |
| ATOM | 7 | C | MET | B | 419 | −7.962 | −74.904 | −36.603 | 1.00 | 52.89 | C |
| ATOM | 8 | O | MET | B | 419 | −7.250 | −75.326 | −35.693 | 1.00 | 52.78 | O |
| ATOM | 1 | N | GLY | B | 420 | −8.624 | −75.707 | −37.431 | 1.00 | 52.86 | N |
| ATOM | 2 | CA | GLY | B | 420 | −8.528 | −77.161 | −37.334 | 1.00 | 52.96 | C |
| ATOM | 3 | C | GLY | B | 420 | −9.803 | −77.821 | −36.838 | 1.00 | 53.03 | C |
| ATOM | 4 | O | GLY | B | 420 | −10.731 | −77.138 | −36.407 | 1.00 | 53.13 | O |
| ATOM | 1 | N | PRO | B | 421 | −9.864 | −79.161 | −36.911 | 1.00 | 53.04 | N |
| ATOM | 2 | CA | PRO | B | 421 | −11.037 | −79.878 | −36.471 | 1.00 | 53.09 | C |
| ATOM | 3 | CB | PRO | B | 421 | −11.173 | −80.952 | −37.548 | 1.00 | 52.99 | C |
| ATOM | 4 | CG | PRO | B | 421 | −9.717 | −81.301 | −37.885 | 1.00 | 52.91 | C |
| ATOM | 5 | CD | PRO | B | 421 | −8.857 | −80.087 | −37.465 | 1.00 | 53.05 | C |
| ATOM | 6 | C | PRO | B | 421 | −10.848 | −80.558 | −35.115 | 1.00 | 53.26 | C |
| ATOM | 7 | O | PRO | B | 421 | −11.742 | −81.294 | −34.668 | 1.00 | 53.35 | O |
| ATOM | 1 | N | PHE | B | 422 | −9.710 | −80.321 | −34.459 | 1.00 | 53.47 | N |
| ATOM | 2 | CA | PHE | B | 422 | −9.361 | −81.125 | −33.284 | 1.00 | 53.71 | C |
| ATOM | 3 | CB | PHE | B | 422 | −7.939 | −81.673 | −33.392 | 1.00 | 53.51 | C |
| ATOM | 4 | CG | PHE | B | 422 | −7.860 | −82.947 | −34.161 | 1.00 | 53.01 | C |
| ATOM | 5 | CD1 | PHE | B | 422 | −7.556 | −82.939 | −35.506 | 1.00 | 52.78 | C |
| ATOM | 6 | CE1 | PHE | B | 422 | −7.500 | −84.113 | −36.221 | 1.00 | 52.83 | C |
| ATOM | 7 | CZ | PHE | B | 422 | −7.760 | −85.312 | −35.596 | 1.00 | 52.98 | C |
| ATOM | 8 | CE2 | PHE | B | 422 | −8.077 | −85.331 | −34.256 | 1.00 | 52.93 | C |
| ATOM | 9 | CD2 | PHE | B | 422 | −8.131 | −84.153 | −33.547 | 1.00 | 52.79 | C |
| ATOM | 10 | C | PHE | B | 422 | −9.606 | −80.504 | −31.913 | 1.00 | 54.16 | C |
| ATOM | 11 | O | PHE | B | 422 | −10.048 | −81.193 | −30.989 | 1.00 | 54.20 | O |
| ATOM | 1 | N | VAL | B | 423 | −9.314 | −79.217 | −31.767 | 1.00 | 54.72 | N |
| ATOM | 2 | CA | VAL | B | 423 | −9.603 | −78.540 | −30.507 | 1.00 | 55.30 | C |
| ATOM | 3 | CB | VAL | B | 423 | −8.329 | −77.988 | −29.828 | 1.00 | 55.35 | C |
| ATOM | 4 | CG1 | VAL | B | 423 | −8.687 | −77.283 | −28.525 | 1.00 | 55.52 | C |
| ATOM | 5 | CG2 | VAL | B | 423 | −7.340 | −79.119 | −29.559 | 1.00 | 55.43 | C |
| ATOM | 6 | C | VAL | B | 423 | −10.633 | −77.434 | −30.704 | 1.00 | 55.57 | C |
| ATOM | 7 | O | VAL | B | 423 | −10.364 | −76.444 | −31.387 | 1.00 | 55.56 | O |
| ATOM | 1 | N | GLU | B | 424 | −11.808 | −77.627 | −30.100 | 1.00 | 56.06 | N |
| ATOM | 2 | CA | GLU | B | 424 | −12.936 | −76.685 | −30.174 | 1.00 | 56.34 | C |
| ATOM | 3 | CB | GLU | B | 424 | −12.605 | −75.352 | −29.463 | 1.00 | 56.44 | C |
| ATOM | 4 | CG | GLU | B | 424 | −13.793 | −74.350 | −29.302 | 1.00 | 56.50 | C |
| ATOM | 5 | CD | GLU | B | 424 | −13.681 | −73.133 | −30.258 | 1.00 | 57.30 | C |
| ATOM | 6 | OE1 | GLU | B | 424 | −12.463 | −72.749 | −30.638 | 1.00 | 57.51 | O |
| ATOM | 7 | OE2 | GLU | B | 424 | −14.750 | −72.545 | −30.636 | 1.00 | 57.40 | O |
| ATOM | 8 | C | GLU | B | 424 | −13.429 | −76.440 | −31.610 | 1.00 | 56.48 | C |
| ATOM | 9 | O | GLU | B | 424 | −12.779 | −75.721 | −32.388 | 1.00 | 56.64 | O |
| TER | 9 | | GLU | B | 424 | | | | | | |
| ATOM | 1 | N | GLU | B | 443 | −0.405 | −85.521 | −2.811 | 1.00 | 65.60 | N |
| ATOM | 2 | CA | GLU | B | 443 | 0.611 | −84.482 | −2.704 | 1.00 | 65.73 | C |
| ATOM | 3 | CB | GLU | B | 443 | 1.290 | −84.527 | −1.330 | 1.00 | 66.04 | C |
| ATOM | 4 | CG | GLU | B | 443 | 2.377 | −83.462 | −1.118 | 1.00 | 67.13 | C |
| ATOM | 5 | CD | GLU | B | 443 | 1.813 | −82.071 | −0.850 | 1.00 | 68.58 | C |
| ATOM | 6 | OE1 | GLU | B | 443 | 0.633 | −81.816 | −1.184 | 1.00 | 69.01 | O |
| ATOM | 7 | OE2 | GLU | B | 443 | 2.558 | −81.229 | −0.300 | 1.00 | 69.27 | O |
| ATOM | 8 | C | GLU | B | 443 | 1.653 | −84.609 | −3.809 | 1.00 | 65.36 | C |
| ATOM | 9 | O | GLU | B | 443 | 1.374 | −84.292 | −4.965 | 1.00 | 65.51 | O |
| ATOM | 1 | N | TRP | B | 444 | 2.848 | −85.071 | −3.442 | 1.00 | 64.83 | N |
| ATOM | 2 | CA | TRP | B | 444 | 3.954 | −85.228 | −4.383 | 1.00 | 64.28 | C |
| ATOM | 3 | CB | TRP | B | 444 | 5.231 | −84.611 | −3.807 | 1.00 | 64.11 | C |
| ATOM | 4 | CG | TRP | B | 444 | 6.319 | −84.393 | −4.820 | 1.00 | 63.96 | C |
| ATOM | 5 | CD1 | TRP | B | 444 | 6.157 | −84.086 | −6.139 | 1.00 | 63.74 | C |
| ATOM | 6 | NE1 | TRP | B | 444 | 7.382 | −83.952 | −6.748 | 1.00 | 63.50 | N |
| ATOM | 7 | CE2 | TRP | B | 444 | 8.367 | −84.164 | −5.821 | 1.00 | 63.49 | C |
| ATOM | 8 | CD2 | TRP | B | 444 | 7.736 | −84.438 | −4.591 | 1.00 | 63.81 | C |
| ATOM | 9 | CE3 | TRP | B | 444 | 8.530 | −84.694 | −3.469 | 1.00 | 63.77 | C |
| ATOM | 10 | CZ3 | TRP | B | 444 | 9.909 | −84.665 | −3.612 | 1.00 | 63.70 | C |
| ATOM | 11 | CH2 | TRP | B | 444 | 10.505 | −84.386 | −4.846 | 1.00 | 63.62 | C |
| ATOM | 12 | CZ2 | TRP | B | 444 | 9.753 | −84.137 | −5.960 | 1.00 | 63.59 | C |
| ATOM | 13 | C | TRP | B | 444 | 4.158 | −86.707 | −4.672 | 1.00 | 64.04 | C |
| ATOM | 14 | O | TRP | B | 444 | 4.079 | −87.531 | −3.767 | 1.00 | 64.09 | O |
| ATOM | 1 | N | VAL | B | 445 | 4.426 | −87.043 | −5.930 | 1.00 | 63.74 | N |
| ATOM | 2 | CA | VAL | B | 445 | 4.422 | −88.445 | −6.349 | 1.00 | 63.47 | C |
| ATOM | 3 | CB | VAL | B | 445 | 4.157 | −88.591 | −7.867 | 1.00 | 63.49 | C |
| ATOM | 4 | CG1 | VAL | B | 445 | 3.400 | −89.891 | −8.137 | 1.00 | 63.40 | C |
| ATOM | 5 | CG2 | VAL | B | 445 | 3.347 | −87.410 | −8.382 | 1.00 | 63.67 | C |
| ATOM | 6 | C | VAL | B | 445 | 5.686 | −89.236 | −5.995 | 1.00 | 63.28 | C |
| ATOM | 7 | O | VAL | B | 445 | 5.652 | −90.466 | −5.953 | 1.00 | 63.22 | O |
| ATOM | 1 | N | VAL | B | 446 | 6.797 | −88.549 | −5.742 | 1.00 | 63.03 | N |
| ATOM | 2 | CA | VAL | B | 446 | 8.068 | −89.251 | −5.538 | 1.00 | 62.82 | C |
| ATOM | 3 | CB | VAL | B | 446 | 9.258 | −88.540 | −6.237 | 1.00 | 62.73 | C |
| ATOM | 4 | CG1 | VAL | B | 446 | 8.775 | −87.739 | −7.436 | 1.00 | 62.74 | C |
| ATOM | 5 | CG2 | VAL | B | 446 | 9.992 | −87.644 | −5.271 | 1.00 | 62.62 | C |
| ATOM | 6 | C | VAL | B | 446 | 8.400 | −89.527 | −4.066 | 1.00 | 62.78 | C |
| ATOM | 7 | O | VAL | B | 446 | 9.343 | −90.263 | −3.764 | 1.00 | 62.73 | O |
| ATOM | 1 | N | THR | B | 447 | 7.616 | −88.954 | −3.155 | 1.00 | 62.71 | N |
| ATOM | 2 | CA | THR | B | 447 | 7.850 | −89.148 | −1.722 | 1.00 | 62.74 | C |
| ATOM | 3 | CB | THR | B | 447 | 6.862 | −88.339 | −0.858 | 1.00 | 62.72 | C |
| ATOM | 4 | OG1 | THR | B | 447 | 5.639 | −88.163 | −1.578 | 1.00 | 63.01 | O |
| ATOM | 5 | CG2 | THR | B | 447 | 7.438 | −86.972 | −0.526 | 1.00 | 62.66 | C |
| ATOM | 6 | C | THR | B | 447 | 7.820 | −90.620 | −1.301 | 1.00 | 62.71 | C |
| ATOM | 7 | O | THR | B | 447 | 8.427 | −90.991 | −0.295 | 1.00 | 62.63 | O |
| ATOM | 1 | N | LYS | B | 448 | 7.121 | −91.453 | −2.070 | 1.00 | 62.74 | N |
| ATOM | 2 | CA | LYS | B | 448 | 7.119 | −92.895 | −1.822 | 1.00 | 62.80 | C |
| ATOM | 3 | CB | LYS | B | 448 | 6.130 | −93.628 | −2.735 | 1.00 | 62.76 | C |
| ATOM | 4 | CG | LYS | B | 448 | 4.711 | −93.108 | −2.686 | 1.00 | 63.05 | C |
| ATOM | 5 | CD | LYS | B | 448 | 4.392 | −92.289 | −3.923 | 1.00 | 63.48 | C |
| ATOM | 6 | CE | LYS | B | 448 | 3.255 | −91.319 | −3.661 | 1.00 | 63.64 | C |
| ATOM | 7 | NZ | LYS | B | 448 | 3.668 | −90.275 | −2.681 | 1.00 | 63.53 | N |
| ATOM | 8 | C | LYS | B | 448 | 8.514 | −93.490 | −2.009 | 1.00 | 62.83 | C |
| ATOM | 9 | O | LYS | B | 448 | 8.957 | −94.314 | −1.209 | 1.00 | 63.01 | O |
| ATOM | 1 | N | ASP | B | 449 | 9.203 | −93.070 | −3.065 | 1.00 | 62.70 | N |
| ATOM | 2 | CA | ASP | B | 449 | 10.507 | −93.632 | −3.390 | 1.00 | 62.64 | C |
| ATOM | 3 | CB | ASP | B | 449 | 10.618 | −93.837 | −4.902 | 1.00 | 62.80 | C |
| ATOM | 4 | CG | ASP | B | 449 | 9.549 | −94.782 | −5.444 | 1.00 | 63.19 | C |
| ATOM | 5 | OD1 | ASP | B | 449 | 9.378 | −95.890 | −4.877 | 1.00 | 63.74 | O |
| ATOM | 6 | OD2 | ASP | B | 449 | 8.881 | −94.419 | −6.439 | 1.00 | 63.29 | O |
| ATOM | 7 | C | ASP | B | 449 | 11.632 | −92.743 | −2.869 | 1.00 | 62.46 | C |
| ATOM | 8 | O | ASP | B | 449 | 12.816 | −92.959 | −3.166 | 1.00 | 62.44 | O |
| ATOM | 1 | N | LYS | B | 450 | 11.247 | −91.756 | −2.066 | 1.00 | 62.30 | N |
| ATOM | 2 | CA | LYS | B | 450 | 12.174 | −90.744 | −1.578 | 1.00 | 62.13 | C |
| ATOM | 3 | CB | LYS | B | 450 | 11.449 | −89.734 | −0.687 | 1.00 | 62.11 | C |
| ATOM | 4 | CG | LYS | B | 450 | 11.805 | −88.274 | −0.967 | 1.00 | 61.77 | C |
| ATOM | 5 | CD | LYS | B | 450 | 13.208 | −87.897 | −0.505 | 1.00 | 60.83 | C |
| ATOM | 6 | CE | LYS | B | 450 | 13.530 | −86.454 | −0.874 | 1.00 | 59.97 | C |
| ATOM | 7 | NZ | LYS | B | 450 | 14.896 | −86.047 | −0.463 | 1.00 | 59.33 | N |
| ATOM | 8 | C | LYS | B | 450 | 13.360 | −91.352 | −0.833 | 1.00 | 62.15 | C |
| ATOM | 9 | O | LYS | B | 450 | 14.487 | −91.248 | −1.294 | 1.00 | 62.07 | O |
| ATOM | 1 | N | SER | B | 451 | 13.106 | −91.983 | 0.310 | 1.00 | 62.16 | N |
| ATOM | 2 | CA | SER | B | 451 | 14.180 | −92.559 | 1.125 | 1.00 | 62.17 | C |
| ATOM | 3 | CB | SER | B | 451 | 13.587 | −93.360 | 2.281 | 1.00 | 62.12 | C |
| ATOM | 4 | OG | SER | B | 451 | 12.298 | −93.834 | 1.938 | 1.00 | 62.06 | O |
| ATOM | 5 | C | SER | B | 451 | 15.154 | −93.415 | 0.303 | 1.00 | 62.26 | C |
| ATOM | 6 | O | SER | B | 451 | 16.370 | −93.404 | 0.536 | 1.00 | 62.26 | O |
| ATOM | 1 | N | LYS | B | 452 | 14.605 | −94.144 | −0.662 | 1.00 | 62.25 | N |
| ATOM | 2 | CA | LYS | B | 452 | 15.375 | −94.923 | −1.626 | 1.00 | 62.40 | C |
| ATOM | 3 | CB | LYS | B | 452 | 14.369 | −95.657 | −2.519 | 1.00 | 62.38 | C |
| ATOM | 4 | CG | LYS | B | 452 | 14.904 | −96.422 | −3.717 | 1.00 | 62.84 | C |
| ATOM | 5 | CD | LYS | B | 452 | 13.773 | −97.330 | −4.253 | 1.00 | 63.07 | C |
| ATOM | 6 | CE | LYS | B | 452 | 13.989 | −97.788 | −5.702 | 1.00 | 64.07 | C |
| ATOM | 7 | NZ | LYS | B | 452 | 13.378 | −96.868 | −6.709 | 1.00 | 63.97 | N |
| ATOM | 8 | C | LYS | B | 452 | 16.312 | −94.016 | −2.449 | 1.00 | 62.22 | C |
| ATOM | 9 | O | LYS | B | 452 | 17.575 | −94.152 | −2.426 | 1.00 | 62.21 | O |
| ATOM | 1 | N | TYR | B | 453 | 15.696 | −93.076 | −3.164 | 1.00 | 62.12 | N |
| ATOM | 2 | CA | TYR | B | 453 | 16.465 | −92.110 | −3.934 | 1.00 | 61.83 | C |
| ATOM | 3 | CB | TYR | B | 453 | 15.541 | −91.057 | −4.531 | 1.00 | 61.96 | C |
| ATOM | 4 | CG | TYR | B | 453 | 14.589 | −91.611 | −5.559 | 1.00 | 62.18 | C |
| ATOM | 5 | CD1 | TYR | B | 453 | 14.878 | −92.789 | −6.231 | 1.00 | 62.45 | C |
| ATOM | 6 | CE1 | TYR | B | 453 | 14.016 | −93.298 | −7.179 | 1.00 | 62.52 | C |
| ATOM | 7 | CZ | TYR | B | 453 | 12.855 | −92.622 | −7.475 | 1.00 | 62.28 | C |
| ATOM | 8 | OH | TYR | B | 453 | 12.004 | −93.133 | −8.419 | 1.00 | 62.68 | O |
| ATOM | 9 | CE2 | TYR | B | 453 | 12.543 | −91.445 | −6.830 | 1.00 | 62.18 | C |
| ATOM | 10 | CD2 | TYR | B | 453 | 13.411 | −90.945 | −5.878 | 1.00 | 62.43 | C |
| ATOM | 11 | C | TYR | B | 453 | 17.547 | −91.449 | −3.082 | 1.00 | 61.73 | C |
| ATOM | 12 | O | TYR | B | 453 | 18.696 | −91.346 | −3.504 | 1.00 | 61.52 | O |
| ATOM | 1 | N | ASP | B | 454 | 17.167 | −91.011 | −1.883 | 1.00 | 61.76 | N |
| ATOM | 2 | CA | ASP | B | 454 | 18.083 | −90.401 | −0.921 | 1.00 | 61.68 | C |
| ATOM | 3 | CB | ASP | B | 454 | 17.359 | −90.082 | 0.386 | 1.00 | 61.75 | C |
| ATOM | 4 | CG | ASP | B | 454 | 16.590 | −88.786 | 0.325 | 1.00 | 62.05 | C |
| ATOM | 5 | OD1 | ASP | B | 454 | 16.033 | −88.378 | 1.367 | 1.00 | 62.30 | O |
| ATOM | 6 | OD2 | ASP | B | 454 | 16.545 | −88.176 | −0.764 | 1.00 | 63.12 | O |
| ATOM | 7 | C | ASP | B | 454 | 19.261 | −91.304 | −0.616 | 1.00 | 61.60 | C |
| ATOM | 8 | O | ASP | B | 454 | 20.408 | −90.860 | −0.647 | 1.00 | 61.46 | O |
| ATOM | 1 | N | GLU | B | 455 | 18.974 | −92.565 | −0.302 | 1.00 | 61.64 | N |
| ATOM | 2 | CA | GLU | B | 455 | 20.038 | −93.540 | −0.108 | 1.00 | 61.50 | C |
| ATOM | 3 | CB | GLU | B | 455 | 19.472 | −94.962 | −0.044 | 1.00 | 61.52 | C |
| ATOM | 4 | CG | GLU | B | 455 | 20.300 | −95.931 | 0.792 | 1.00 | 61.62 | C |
| ATOM | 5 | CD | GLU | B | 455 | 19.026 | −96.015 | 1.970 | 0.00 | 50.00 | C |
| ATOM | 6 | OE1 | GLU | B | 455 | 19.504 | −96.674 | 2.912 | 0.00 | 50.00 | O |
| ATOM | 7 | OE2 | GLU | B | 455 | 18.049 | −95.268 | 2.084 | 0.00 | 50.00 | O |
| ATOM | 8 | C | GLU | B | 455 | 21.043 | −93.399 | −1.253 | 1.00 | 61.47 | C |
| ATOM | 9 | O | GLU | B | 455 | 22.270 | −93.244 | −1.009 | 1.00 | 61.39 | O |
| ATOM | 1 | N | ILE | B | 456 | 20.534 | −93.395 | −2.504 | 1.00 | 61.57 | N |
| ATOM | 2 | CA | ILE | B | 456 | 21.505 | −93.246 | −3.620 | 1.00 | 61.40 | C |
| ATOM | 3 | CB | ILE | B | 456 | 20.873 | −93.420 | −5.034 | 1.00 | 61.41 | C |
| ATOM | 4 | CG1 | ILE | B | 456 | 20.581 | −94.894 | −5.336 | 1.00 | 61.57 | C |
| ATOM | 5 | CD1 | ILE | B | 456 | 19.280 | −95.411 | −4.766 | 1.00 | 61.87 | C |
| ATOM | 6 | CG2 | ILE | B | 456 | 21.819 | −92.913 | −6.108 | 1.00 | 60.97 | C |
| ATOM | 7 | C | ILE | B | 456 | 22.232 | −91.892 | −3.557 | 1.00 | 61.33 | C |
| ATOM | 8 | O | ILE | B | 456 | 23.473 | −91.817 | −3.487 | 1.00 | 61.32 | O |
| ATOM | 1 | N | PHE | B | 457 | 21.439 | −90.826 | −3.568 | 1.00 | 61.28 | N |
| ATOM | 2 | CA | PHE | B | 457 | 21.951 | −89.462 | −3.579 | 1.00 | 61.18 | C |
| ATOM | 3 | CB | PHE | B | 457 | 20.825 | −88.478 | −3.264 | 1.00 | 61.10 | C |
| ATOM | 4 | CG | PHE | B | 457 | 21.288 | −87.059 | −3.078 | 1.00 | 61.06 | C |
| ATOM | 5 | CD1 | PHE | B | 457 | 21.514 | −86.240 | −4.173 | 1.00 | 60.72 | C |
| ATOM | 6 | CE1 | PHE | B | 457 | 21.925 | −84.930 | −4.005 | 1.00 | 60.37 | C |
| ATOM | 7 | CZ | PHE | B | 457 | 22.109 | −84.424 | −2.737 | 1.00 | 60.51 | C |
| ATOM | 8 | CE2 | PHE | B | 457 | 21.884 | −85.228 | −1.635 | 1.00 | 60.56 | C |
| ATOM | 9 | CD2 | PHE | B | 457 | 21.476 | −86.536 | −1.806 | 1.00 | 60.85 | C |
| ATOM | 10 | C | PHE | B | 457 | 23.083 | −89.275 | −2.588 | 1.00 | 61.08 | C |
| ATOM | 11 | O | PHE | B | 457 | 24.142 | −88.746 | −2.928 | 1.00 | 61.04 | O |
| ATOM | 1 | N | TYR | B | 458 | 22.852 | −89.710 | −1.357 | 1.00 | 61.12 | N |
| ATOM | 2 | CA | TYR | B | 458 | 23.845 | −89.535 | −0.313 | 1.00 | 60.96 | C |
| ATOM | 3 | CB | TYR | B | 458 | 23.207 | −89.605 | 1.071 | 1.00 | 60.87 | C |
| ATOM | 4 | CG | TYR | B | 458 | 22.380 | −88.380 | 1.374 | 1.00 | 60.78 | C |
| ATOM | 5 | CD1 | TYR | B | 458 | 22.977 | −87.135 | 1.493 | 1.00 | 60.62 | C |
| ATOM | 6 | CE1 | TYR | B | 458 | 22.233 | −86.009 | 1.761 | 1.00 | 60.72 | C |
| ATOM | 7 | CZ | TYR | B | 458 | 20.870 | −86.115 | 1.910 | 1.00 | 60.91 | C |
| ATOM | 8 | OH | TYR | B | 458 | 20.124 | −84.987 | 2.178 | 1.00 | 61.14 | O |
| ATOM | 9 | CE2 | TYR | B | 458 | 20.250 | −87.340 | 1.793 | 1.00 | 60.97 | C |
| ATOM | 10 | CD2 | TYR | B | 458 | 21.005 | −88.463 | 1.521 | 1.00 | 60.82 | C |
| ATOM | 11 | C | TYR | B | 458 | 24.997 | −90.509 | −0.460 | 1.00 | 60.98 | C |
| ATOM | 12 | O | TYR | B | 458 | 26.113 | −90.212 | −0.039 | 1.00 | 60.99 | O |
| ATOM | 1 | N | ASN | B | 459 | 24.746 | −91.663 | −1.076 | 1.00 | 61.12 | N |
| ATOM | 2 | CA | ASN | B | 459 | 25.874 | −92.529 | −1.426 | 1.00 | 61.04 | C |
| ATOM | 3 | CB | ASN | B | 459 | 25.435 | −93.967 | −1.690 | 1.00 | 61.02 | C |
| ATOM | 4 | CG | ASN | B | 459 | 25.682 | −94.868 | −0.501 | 1.00 | 61.15 | C |
| ATOM | 5 | OD1 | ASN | B | 459 | 24.853 | −95.713 | −0.163 | 1.00 | 61.72 | O |
| ATOM | 6 | ND2 | ASN | B | 459 | 26.826 | −94.682 | 0.152 | 1.00 | 61.27 | N |
| ATOM | 7 | C | ASN | B | 459 | 26.725 | −91.986 | −2.573 | 1.00 | 61.02 | C |
| ATOM | 8 | O | ASN | B | 459 | 27.807 | −92.503 | −2.846 | 1.00 | 60.90 | O |
| ATOM | 1 | N | LEU | B | 460 | 26.238 | −90.936 | −3.234 | 1.00 | 61.16 | N |
| ATOM | 2 | CA | LEU | B | 460 | 27.036 | −90.256 | −4.269 | 1.00 | 61.21 | C |
| ATOM | 3 | CB | LEU | B | 460 | 26.144 | −89.735 | −5.399 | 1.00 | 61.18 | C |
| ATOM | 4 | CG | LEU | B | 460 | 25.941 | −90.651 | −6.607 | 1.00 | 61.31 | C |
| ATOM | 5 | CD1 | LEU | B | 460 | 25.626 | −92.081 | −6.188 | 1.00 | 61.70 | C |
| ATOM | 6 | CD2 | LEU | B | 460 | 24.853 | −90.103 | −7.519 | 1.00 | 61.36 | C |
| ATOM | 7 | C | LEU | B | 460 | 27.927 | −89.118 | −3.751 | 1.00 | 61.21 | C |
| ATOM | 8 | O | LEU | B | 460 | 28.542 | −88.401 | −4.542 | 1.00 | 61.15 | O |
| ATOM | 1 | N | ALA | B | 461 | 27.990 | −88.958 | −2.431 | 1.00 | 61.30 | N |
| ATOM | 2 | CA | ALA | B | 461 | 28.832 | −87.933 | −1.791 | 1.00 | 61.39 | C |
| ATOM | 3 | CB | ALA | B | 461 | 30.310 | −88.328 | −1.848 | 1.00 | 61.32 | C |
| ATOM | 4 | C | ALA | B | 461 | 28.632 | −86.526 | −2.356 | 1.00 | 61.46 | C |
| ATOM | 5 | O | ALA | B | 461 | 29.489 | −86.016 | −3.080 | 1.00 | 61.46 | O |
| ATOM | 1 | N | PRO | B | 462 | 27.498 | −85.891 | −2.015 | 1.00 | 61.52 | N |
| ATOM | 2 | CA | PRO | B | 462 | 27.192 | −84.538 | −2.456 | 1.00 | 61.50 | C |
| ATOM | 3 | CB | PRO | B | 462 | 25.672 | −84.439 | −2.278 | 1.00 | 61.48 | C |
| ATOM | 4 | CG | PRO | B | 462 | 25.224 | −85.764 | −1.701 | 1.00 | 61.39 | C |
| ATOM | 5 | CD | PRO | B | 462 | 26.429 | −86.437 | −1.167 | 1.00 | 61.48 | C |
| ATOM | 6 | C | PRO | B | 462 | 27.888 | −83.514 | −1.573 | 1.00 | 61.55 | C |
| ATOM | 7 | O | PRO | B | 462 | 28.433 | −83.873 | −0.532 | 1.00 | 61.39 | O |
| ATOM | 1 | N | ALA | B | 463 | 27.859 | −82.252 | −1.992 | 1.00 | 61.78 | N |
| ATOM | 2 | CA | ALA | B | 463 | 28.567 | −81.176 | −1.301 | 1.00 | 61.99 | C |
| ATOM | 3 | CB | ALA | B | 463 | 28.388 | −79.860 | −2.043 | 1.00 | 62.09 | C |
| ATOM | 4 | C | ALA | B | 463 | 28.111 | −81.031 | 0.140 | 1.00 | 62.16 | C |
| ATOM | 5 | O | ALA | B | 463 | 28.733 | −81.565 | 1.055 | 1.00 | 62.42 | O |
| ATOM | 1 | N | ASP | B | 464 | 27.028 | −80.293 | 0.343 | 1.00 | 62.26 | N |
| ATOM | 2 | CA | ASP | B | 464 | 26.459 | −80.154 | 1.673 | 1.00 | 62.40 | C |
| ATOM | 3 | CB | ASP | B | 464 | 26.755 | −78.767 | 2.250 | 1.00 | 62.60 | C |
| ATOM | 4 | CG | ASP | B | 464 | 26.038 | −77.651 | 1.502 | 1.00 | 63.34 | C |
| ATOM | 5 | OD1 | ASP | B | 464 | 26.134 | −76.485 | 1.952 | 1.00 | 63.76 | O |
| ATOM | 6 | OD2 | ASP | B | 464 | 25.380 | −77.936 | 0.471 | 1.00 | 64.06 | O |
| ATOM | 7 | C | ASP | B | 464 | 24.964 | −80.382 | 1.585 | 1.00 | 62.23 | C |
| ATOM | 8 | O | ASP | B | 464 | 24.207 | −79.934 | 2.434 | 1.00 | 62.39 | O |
| ATOM | 1 | N | GLY | B | 465 | 24.549 | −81.083 | 0.541 | 1.00 | 62.02 | N |
| ATOM | 2 | CA | GLY | B | 465 | 23.140 | −81.324 | 0.294 | 1.00 | 61.82 | C |
| ATOM | 3 | C | GLY | B | 465 | 22.898 | −81.127 | −1.183 | 1.00 | 61.70 | C |
| ATOM | 4 | O | GLY | B | 465 | 21.784 | −81.310 | −1.678 | 1.00 | 61.73 | O |
| ATOM | 1 | N | LYS | B | 466 | 23.960 | −80.752 | −1.887 | 1.00 | 61.43 | N |
| ATOM | 2 | CA | LYS | B | 466 | 23.879 | −80.507 | −3.311 | 1.00 | 61.22 | C |
| ATOM | 3 | CB | LYS | B | 466 | 24.071 | −79.023 | −3.604 | 1.00 | 61.21 | C |
| ATOM | 4 | CG | LYS | B | 466 | 22.937 | −78.154 | −3.108 | 1.00 | 61.05 | C |
| ATOM | 5 | CD | LYS | B | 466 | 23.345 | −76.698 | −3.052 | 1.00 | 61.32 | C |
| ATOM | 6 | CE | LYS | B | 466 | 22.293 | −75.881 | −2.334 | 1.00 | 61.80 | C |
| ATOM | 7 | NZ | LYS | B | 466 | 21.944 | −76.494 | −1.019 | 1.00 | 62.20 | N |
| ATOM | 8 | C | LYS | B | 466 | 24.917 | −81.323 | −4.053 | 1.00 | 61.12 | C |
| ATOM | 9 | O | LYS | B | 466 | 26.113 | −81.218 | −3.792 | 1.00 | 61.13 | O |
| ATOM | 1 | N | LEU | B | 467 | 24.448 | −82.144 | −4.980 | 1.00 | 61.00 | N |
| ATOM | 2 | CA | LEU | B | 467 | 25.341 | −82.926 | −5.809 | 1.00 | 60.88 | C |
| ATOM | 3 | CB | LEU | B | 467 | 24.618 | −84.164 | −6.341 | 1.00 | 60.79 | C |
| ATOM | 4 | CG | LEU | B | 467 | 25.461 | −85.111 | −7.190 | 1.00 | 60.87 | C |
| ATOM | 5 | CD1 | LEU | B | 467 | 26.613 | −85.658 | −6.365 | 1.00 | 61.30 | C |
| ATOM | 6 | CD2 | LEU | B | 467 | 24.614 | −86.240 | −7.743 | 1.00 | 60.91 | C |
| ATOM | 7 | C | LEU | B | 467 | 25.839 | −82.065 | −6.966 | 1.00 | 60.83 | C |
| ATOM | 8 | O | LEU | B | 467 | 25.044 | −81.491 | −7.712 | 1.00 | 60.86 | O |
| ATOM | 1 | N | SER | B | 468 | 27.158 | −81.966 | −7.099 | 1.00 | 60.71 | N |
| ATOM | 2 | CA | SER | B | 468 | 27.777 | −81.277 | −8.227 | 1.00 | 60.55 | C |
| ATOM | 3 | CB | SER | B | 468 | 29.287 | −81.522 | −8.211 | 1.00 | 60.68 | C |
| ATOM | 4 | OG | SER | B | 468 | 29.806 | −81.658 | −9.524 | 1.00 | 61.03 | O |
| ATOM | 5 | C | SER | B | 468 | 27.182 | −81.727 | −9.564 | 1.00 | 60.34 | C |
| ATOM | 6 | O | SER | B | 468 | 26.679 | −82.846 | −9.682 | 1.00 | 60.29 | O |
| ATOM | 1 | N | GLY | B | 469 | 27.246 | −80.852 | −10.565 | 1.00 | 60.13 | N |
| ATOM | 2 | CA | GLY | B | 469 | 26.713 | −81.153 | −11.894 | 1.00 | 59.92 | C |
| ATOM | 3 | C | GLY | B | 469 | 27.448 | −82.273 | −12.611 | 1.00 | 59.86 | C |
| ATOM | 4 | O | GLY | B | 469 | 26.835 | −83.108 | −13.278 | 1.00 | 59.75 | O |
| ATOM | 1 | N | SER | B | 470 | 28.767 | −82.298 | −12.471 | 1.00 | 59.92 | N |
| ATOM | 2 | CA | SER | B | 470 | 29.580 | −83.316 | −13.128 | 1.00 | 60.13 | C |
| ATOM | 3 | CB | SER | B | 470 | 31.070 | −82.996 | −12.972 | 1.00 | 60.18 | C |
| ATOM | 4 | OG | SER | B | 470 | 31.409 | −82.804 | −11.610 | 1.00 | 60.39 | O |
| ATOM | 5 | C | SER | B | 470 | 29.271 | −84.730 | −12.621 | 1.00 | 60.16 | C |
| ATOM | 6 | O | SER | B | 470 | 29.128 | −85.667 | −13.415 | 1.00 | 60.19 | O |
| ATOM | 1 | N | LYS | B | 471 | 29.165 | −84.875 | −11.301 | 1.00 | 60.16 | N |
| ATOM | 2 | CA | LYS | B | 471 | 28.853 | −86.160 | −10.680 | 1.00 | 60.23 | C |
| ATOM | 3 | CB | LYS | B | 471 | 28.918 | −86.032 | −9.157 | 1.00 | 60.32 | C |
| ATOM | 4 | CG | LYS | B | 471 | 29.715 | −84.826 | −8.674 | 1.00 | 60.75 | C |
| ATOM | 5 | CD | LYS | B | 471 | 31.190 | −85.140 | −8.471 | 1.00 | 61.90 | C |
| ATOM | 6 | CE | LYS | B | 471 | 31.583 | −85.072 | −6.994 | 1.00 | 62.37 | C |
| ATOM | 7 | NZ | LYS | B | 471 | 30.749 | −85.959 | −6.130 | 1.00 | 62.88 | N |
| ATOM | 8 | C | LYS | B | 471 | 27.452 | −86.600 | −11.106 | 1.00 | 60.16 | C |
| ATOM | 9 | O | LYS | B | 471 | 27.223 | −87.758 | −11.515 | 1.00 | 60.14 | O |
| ATOM | 1 | N | ALA | B | 472 | 26.521 | −85.654 | −11.014 | 1.00 | 60.13 | N |
| ATOM | 2 | CA | ALA | B | 472 | 25.155 | −85.868 | −11.458 | 1.00 | 60.03 | C |
| ATOM | 3 | CB | ALA | B | 472 | 24.353 | −84.582 | −11.354 | 1.00 | 59.90 | C |
| ATOM | 4 | C | ALA | B | 472 | 25.170 | −86.382 | −12.890 | 1.00 | 59.97 | C |
| ATOM | 5 | O | ALA | B | 472 | 24.488 | −87.358 | −13.218 | 1.00 | 60.05 | O |
| ATOM | 1 | N | LYS | B | 473 | 25.966 | −85.741 | −13.739 | 1.00 | 59.85 | N |
| ATOM | 2 | CA | LYS | B | 473 | 26.063 | −86.203 | −15.112 | 1.00 | 59.96 | C |
| ATOM | 3 | CB | LYS | B | 473 | 26.893 | −85.269 | −15.988 | 1.00 | 59.91 | C |
| ATOM | 4 | CG | LYS | B | 473 | 26.875 | −85.683 | −17.455 | 1.00 | 59.67 | C |
| ATOM | 5 | CD | LYS | B | 473 | 27.769 | −84.800 | −18.309 | 1.00 | 59.75 | C |
| ATOM | 6 | CE | LYS | B | 473 | 27.437 | −84.943 | −19.788 | 1.00 | 59.64 | C |
| ATOM | 7 | NZ | LYS | B | 473 | 28.334 | −84.122 | −20.649 | 1.00 | 59.71 | N |
| ATOM | 8 | C | LYS | B | 473 | 26.611 | −87.620 | −15.183 | 1.00 | 60.14 | C |
| ATOM | 9 | O | LYS | B | 473 | 25.941 | −88.495 | −15.719 | 1.00 | 60.26 | O |
| ATOM | 1 | N | THR | B | 474 | 27.806 | −87.866 | −14.640 | 1.00 | 60.16 | N |
| ATOM | 2 | CA | THR | B | 474 | 28.372 | −89.219 | −14.739 | 1.00 | 60.24 | C |
| ATOM | 3 | CB | THR | B | 474 | 29.757 | −89.402 | −14.031 | 1.00 | 60.23 | C |
| ATOM | 4 | OG1 | THR | B | 474 | 29.800 | −88.662 | −12.808 | 1.00 | 60.62 | O |
| ATOM | 5 | CG2 | THR | B | 474 | 30.894 | −88.933 | −14.931 | 1.00 | 60.37 | C |
| ATOM | 6 | C | THR | B | 474 | 27.364 | −90.289 | −14.301 | 1.00 | 60.23 | C |
| ATOM | 7 | O | THR | B | 474 | 27.219 | −91.319 | −14.966 | 1.00 | 60.25 | O |
| ATOM | 1 | N | TRP | B | 475 | 26.645 | −90.037 | −13.211 | 1.00 | 60.28 | N |
| ATOM | 2 | CA | TRP | B | 475 | 25.606 | −90.978 | −12.787 | 1.00 | 60.42 | C |
| ATOM | 3 | CB | TRP | B | 475 | 25.023 | −90.535 | −11.445 | 1.00 | 60.84 | C |
| ATOM | 4 | CG | TRP | B | 475 | 23.813 | −91.301 | −11.013 | 1.00 | 61.18 | C |
| ATOM | 5 | CD1 | TRP | B | 475 | 23.775 | −92.585 | −10.565 | 1.00 | 61.70 | C |
| ATOM | 6 | NE1 | TRP | B | 475 | 22.485 | −92.942 | −10.256 | 1.00 | 61.88 | N |
| ATOM | 7 | CE2 | TRP | B | 475 | 21.660 | −91.878 | −10.500 | 1.00 | 61.50 | C |
| ATOM | 8 | CD2 | TRP | B | 475 | 22.463 | −90.823 | −10.976 | 1.00 | 61.57 | C |
| ATOM | 9 | CE3 | TRP | B | 475 | 21.854 | −89.606 | −11.306 | 1.00 | 62.24 | C |
| ATOM | 10 | CZ3 | TRP | B | 475 | 20.477 | −89.484 | −11.149 | 1.00 | 62.05 | C |
| ATOM | 11 | CH2 | TRP | B | 475 | 19.707 | −90.555 | −10.671 | 1.00 | 61.82 | C |
| ATOM | 12 | CZ2 | TRP | B | 475 | 20.280 | −91.756 | −10.343 | 1.00 | 61.71 | C |
| ATOM | 13 | C | TRP | B | 475 | 24.498 | −91.098 | −13.840 | 1.00 | 60.37 | C |
| ATOM | 14 | O | TRP | B | 475 | 24.152 | −92.206 | −14.315 | 1.00 | 60.28 | O |
| ATOM | 1 | N | MET | B | 476 | 23.944 | −89.948 | −14.209 | 1.00 | 60.24 | N |
| ATOM | 2 | CA | MET | B | 476 | 22.858 | −89.927 | −15.166 | 1.00 | 60.43 | C |
| ATOM | 3 | CB | MET | B | 476 | 22.436 | −88.492 | −15.483 | 1.00 | 60.35 | C |
| ATOM | 4 | CG | MET | B | 476 | 21.636 | −87.837 | −14.364 | 1.00 | 60.65 | C |
| ATOM | 5 | SD | MET | B | 476 | 21.109 | −86.145 | −14.729 | 1.00 | 60.97 | S |
| ATOM | 6 | CE | MET | B | 476 | 19.647 | −86.452 | −15.725 | 1.00 | 61.19 | C |
| ATOM | 7 | C | MET | B | 476 | 23.239 | −90.692 | −16.431 | 1.00 | 60.37 | C |
| ATOM | 8 | O | MET | B | 476 | 22.549 | −91.632 | −16.828 | 1.00 | 60.37 | O |
| ATOM | 1 | N | VAL | B | 477 | 24.347 | −90.304 | −17.053 | 1.00 | 60.31 | N |
| ATOM | 2 | CA | VAL | B | 477 | 24.808 | −90.980 | −18.253 | 1.00 | 60.18 | C |
| ATOM | 3 | CB | VAL | B | 477 | 26.072 | −90.317 | −18.844 | 1.00 | 60.16 | C |
| ATOM | 4 | CG1 | VAL | B | 477 | 27.334 | −91.039 | −18.386 | 1.00 | 60.18 | C |
| ATOM | 5 | CG2 | VAL | B | 477 | 25.991 | −90.304 | −20.364 | 1.00 | 60.61 | C |
| ATOM | 6 | C | VAL | B | 477 | 25.053 | −92.442 | −17.904 | 1.00 | 60.04 | C |
| ATOM | 7 | O | VAL | B | 477 | 24.963 | −93.322 | −18.759 | 1.00 | 59.92 | O |
| ATOM | 1 | N | GLY | B | 478 | 25.336 | −92.689 | −16.628 | 1.00 | 60.05 | N |
| ATOM | 2 | CA | GLY | B | 478 | 25.437 | −94.048 | −16.110 | 1.00 | 59.87 | C |
| ATOM | 3 | C | GLY | B | 478 | 24.152 | −94.826 | −16.310 | 1.00 | 59.75 | C |
| ATOM | 4 | O | GLY | B | 478 | 24.183 | −96.039 | −16.508 | 1.00 | 59.64 | O |
| ATOM | 1 | N | THR | B | 479 | 23.012 | −94.139 | −16.265 | 1.00 | 59.79 | N |
| ATOM | 2 | CA | THR | B | 479 | 21.730 | −94.827 | −16.546 | 1.00 | 59.54 | C |
| ATOM | 3 | CB | THR | B | 479 | 20.512 | −93.965 | −16.201 | 1.00 | 59.50 | C |
| ATOM | 4 | OG1 | THR | B | 479 | 20.208 | −93.109 | −17.311 | 1.00 | 59.40 | O |
| ATOM | 5 | CG2 | THR | B | 479 | 20.774 | −93.142 | −14.953 | 1.00 | 59.72 | C |
| ATOM | 6 | C | THR | B | 479 | 21.544 | −95.280 | −18.002 | 1.00 | 59.41 | C |
| ATOM | 7 | O | THR | B | 479 | 20.429 | −95.606 | −18.416 | 1.00 | 59.36 | O |
| ATOM | 1 | N | LYS | B | 480 | 22.626 | −95.279 | −18.772 | 1.00 | 59.24 | N |
| ATOM | 2 | CA | LYS | B | 480 | 22.602 | −95.706 | −20.175 | 1.00 | 59.00 | C |
| ATOM | 3 | CB | LYS | B | 480 | 22.607 | −97.236 | −20.281 | 1.00 | 58.90 | C |
| ATOM | 4 | CG | LYS | B | 480 | 24.220 | −97.582 | −20.411 | 0.00 | 50.00 | C |
| ATOM | 5 | CD | LYS | B | 480 | 24.384 | −99.002 | −19.919 | 0.00 | 50.00 | C |
| ATOM | 6 | CE | LYS | B | 480 | 23.123 | −99.825 | −20.052 | 0.00 | 50.00 | C |
| ATOM | 7 | NZ | LYS | B | 480 | 22.663 | −100.001 | −21.452 | 0.00 | 50.00 | N |
| ATOM | 8 | C | LYS | B | 480 | 21.452 | −95.120 | −21.000 | 1.00 | 58.86 | C |
| ATOM | 9 | O | LYS | B | 480 | 20.932 | −95.778 | −21.898 | 1.00 | 59.02 | O |
| ATOM | 1 | N | LEU | B | 481 | 21.062 | −93.884 | −20.699 | 1.00 | 58.63 | N |
| ATOM | 2 | CA | LEU | B | 481 | 20.089 | −93.169 | −21.522 | 1.00 | 58.40 | C |
| ATOM | 3 | CB | LEU | B | 481 | 19.144 | −92.336 | −20.650 | 1.00 | 58.41 | C |
| ATOM | 4 | CG | LEU | B | 481 | 17.948 | −93.045 | −20.013 | 1.00 | 58.17 | C |
| ATOM | 5 | CD1 | LEU | B | 481 | 17.016 | −92.033 | −19.380 | 1.00 | 57.72 | C |
| ATOM | 6 | CD2 | LEU | B | 481 | 17.200 | −93.871 | −21.043 | 1.00 | 57.96 | C |
| ATOM | 7 | C | LEU | B | 481 | 20.803 | −92.264 | −22.524 | 1.00 | 58.27 | C |
| ATOM | 8 | O | LEU | B | 481 | 21.929 | −91.835 | −22.276 | 1.00 | 58.35 | O |
| ATOM | 1 | N | PRO | B | 482 | 20.156 | −91.980 | −23.667 | 1.00 | 58.15 | N |
| ATOM | 2 | CA | PRO | B | 482 | 20.739 | −91.080 | −24.662 | 1.00 | 58.10 | C |
| ATOM | 3 | CB | PRO | B | 482 | 19.732 | −91.119 | −25.815 | 1.00 | 58.03 | C |
| ATOM | 4 | CG | PRO | B | 482 | 18.465 | −91.591 | −25.212 | 1.00 | 58.03 | C |
| ATOM | 5 | CD | PRO | B | 482 | 18.849 | −92.507 | −24.096 | 1.00 | 58.12 | C |
| ATOM | 6 | C | PRO | B | 482 | 20.889 | −89.658 | −24.134 | 1.00 | 58.13 | C |
| ATOM | 7 | O | PRO | B | 482 | 20.023 | −89.170 | −23.407 | 1.00 | 58.05 | O |
| ATOM | 1 | N | ASN | B | 483 | 21.990 | −89.010 | −24.508 | 1.00 | 58.25 | N |
| ATOM | 2 | CA | ASN | B | 483 | 22.326 | −87.673 | −24.018 | 1.00 | 58.27 | C |
| ATOM | 3 | CB | ASN | B | 483 | 23.466 | −87.069 | −24.846 | 1.00 | 58.41 | C |
| ATOM | 4 | CG | ASN | B | 483 | 24.180 | −85.936 | −24.122 | 1.00 | 58.98 | C |
| ATOM | 5 | OD1 | ASN | B | 483 | 24.148 | −84.782 | −24.560 | 1.00 | 58.96 | O |
| ATOM | 6 | ND2 | ASN | B | 483 | 24.828 | −86.264 | −23.005 | 1.00 | 59.69 | N |
| ATOM | 7 | C | ASN | B | 483 | 21.130 | −86.730 | −24.011 | 1.00 | 57.99 | C |
| ATOM | 8 | O | ASN | B | 483 | 20.749 | −86.219 | −22.971 | 1.00 | 57.84 | O |
| ATOM | 1 | N | SER | B | 484 | 20.548 | −86.515 | −25.185 | 1.00 | 57.91 | N |
| ATOM | 2 | CA | SER | B | 484 | 19.395 | −85.634 | −25.365 | 1.00 | 57.86 | C |
| ATOM | 3 | CB | SER | B | 484 | 18.779 | −85.891 | −26.746 | 1.00 | 57.83 | C |
| ATOM | 4 | OG | SER | B | 484 | 17.367 | −85.789 | −26.726 | 1.00 | 58.63 | O |
| ATOM | 5 | C | SER | B | 484 | 18.351 | −85.771 | −24.249 | 1.00 | 57.69 | C |
| ATOM | 6 | O | SER | B | 484 | 18.038 | −84.801 | −23.542 | 1.00 | 57.89 | O |
| ATOM | 1 | N | VAL | B | 485 | 17.823 | −86.978 | −24.085 | 1.00 | 57.50 | N |
| ATOM | 2 | CA | VAL | B | 485 | 16.868 | −87.246 | −23.025 | 1.00 | 57.27 | C |
| ATOM | 3 | CB | VAL | B | 485 | 16.534 | −88.746 | −22.933 | 1.00 | 57.16 | C |
| ATOM | 4 | CG1 | VAL | B | 485 | 15.787 | −89.050 | −21.650 | 1.00 | 56.97 | C |
| ATOM | 5 | CG2 | VAL | B | 485 | 15.713 | −89.173 | −24.133 | 1.00 | 56.96 | C |
| ATOM | 6 | C | VAL | B | 485 | 17.425 | −86.756 | −21.697 | 1.00 | 57.35 | C |
| ATOM | 7 | O | VAL | B | 485 | 16.803 | −85.944 | −21.017 | 1.00 | 57.43 | O |
| ATOM | 1 | N | LEU | B | 486 | 18.613 | −87.233 | −21.341 | 1.00 | 57.54 | N |
| ATOM | 2 | CA | LEU | B | 486 | 19.239 | −86.843 | −20.080 | 1.00 | 57.77 | C |
| ATOM | 3 | CB | LEU | B | 486 | 20.638 | −87.454 | −19.946 | 1.00 | 57.88 | C |
| ATOM | 4 | CG | LEU | B | 486 | 20.729 | −88.972 | −19.774 | 1.00 | 58.42 | C |
| ATOM | 5 | CD1 | LEU | B | 486 | 22.172 | −89.447 | −19.904 | 1.00 | 58.66 | C |
| ATOM | 6 | CD2 | LEU | B | 486 | 20.138 | −89.407 | −18.439 | 1.00 | 58.90 | C |
| ATOM | 7 | C | LEU | B | 486 | 19.303 | −85.325 | −19.908 | 1.00 | 57.80 | C |
| ATOM | 8 | O | LEU | B | 486 | 18.955 | −84.808 | −18.850 | 1.00 | 57.86 | O |
| ATOM | 1 | N | GLY | B | 487 | 19.749 | −84.618 | −20.945 | 1.00 | 57.71 | N |
| ATOM | 2 | CA | GLY | B | 487 | 19.857 | −83.163 | −20.904 | 1.00 | 57.64 | C |
| ATOM | 3 | C | GLY | B | 487 | 18.516 | −82.531 | −20.601 | 1.00 | 57.70 | C |
| ATOM | 4 | O | GLY | B | 487 | 18.406 | −81.638 | −19.741 | 1.00 | 57.71 | O |
| ATOM | 1 | N | ARG | B | 488 | 17.488 | −83.010 | −21.298 | 1.00 | 57.78 | N |
| ATOM | 2 | CA | ARG | B | 488 | 16.128 | −82.544 | −21.042 | 1.00 | 57.97 | C |
| ATOM | 3 | CB | ARG | B | 488 | 15.152 | −83.209 | −22.013 | 1.00 | 58.06 | C |
| ATOM | 4 | CG | ARG | B | 488 | 13.971 | −82.338 | −22.415 | 1.00 | 59.39 | C |
| ATOM | 5 | CD | ARG | B | 488 | 13.610 | −82.573 | −23.881 | 1.00 | 61.62 | C |
| ATOM | 6 | NE | ARG | B | 488 | 13.092 | −83.923 | −24.122 | 1.00 | 63.12 | N |
| ATOM | 7 | CZ | ARG | B | 488 | 13.449 | −84.695 | −25.150 | 1.00 | 63.38 | C |
| ATOM | 8 | NH1 | ARG | B | 488 | 12.920 | −85.907 | −25.285 | 1.00 | 63.40 | N |
| ATOM | 9 | NH2 | ARG | B | 488 | 14.347 | −84.266 | −26.033 | 1.00 | 63.17 | N |
| ATOM | 10 | C | ARG | B | 488 | 15.727 | −82.814 | −19.583 | 1.00 | 57.81 | C |
| ATOM | 11 | O | ARG | B | 488 | 15.145 | −81.947 | −18.904 | 1.00 | 57.77 | O |
| ATOM | 1 | N | ILE | B | 489 | 16.064 | −84.008 | −19.099 | 1.00 | 57.58 | N |
| ATOM | 2 | CA | ILE | B | 489 | 15.757 | −84.377 | −17.724 | 1.00 | 57.48 | C |
| ATOM | 3 | CB | ILE | B | 489 | 16.163 | −85.815 | −17.413 | 1.00 | 57.33 | C |
| ATOM | 4 | CG1 | ILE | B | 489 | 15.256 | −86.784 | −18.167 | 1.00 | 57.24 | C |
| ATOM | 5 | CD1 | ILE | B | 489 | 15.547 | −88.239 | −17.903 | 1.00 | 57.50 | C |
| ATOM | 6 | CG2 | ILE | B | 489 | 16.068 | −86.066 | −15.925 | 1.00 | 57.45 | C |
| ATOM | 7 | C | ILE | B | 489 | 16.453 | −83.441 | −16.755 | 1.00 | 57.56 | C |
| ATOM | 8 | O | ILE | B | 489 | 15.844 | −82.935 | −15.813 | 1.00 | 57.63 | O |
| ATOM | 1 | N | TRP | B | 490 | 17.736 | −83.206 | −16.988 | 1.00 | 57.67 | N |
| ATOM | 2 | CA | TRP | B | 490 | 18.470 | −82.265 | −16.172 | 1.00 | 57.64 | C |
| ATOM | 3 | CB | TRP | B | 490 | 19.867 | −82.008 | −16.731 | 1.00 | 57.40 | C |
| ATOM | 4 | CG | TRP | B | 490 | 20.498 | −80.874 | −16.021 | 1.00 | 57.22 | C |
| ATOM | 5 | CD1 | TRP | B | 490 | 20.466 | −79.563 | −16.383 | 1.00 | 56.97 | C |
| ATOM | 6 | NE1 | TRP | B | 490 | 21.128 | −78.800 | −15.453 | 1.00 | 57.04 | N |
| ATOM | 7 | CE2 | TRP | B | 490 | 21.592 | −79.619 | −14.458 | 1.00 | 57.10 | C |
| ATOM | 8 | CD2 | TRP | B | 490 | 21.204 | −80.933 | −14.780 | 1.00 | 57.28 | C |
| ATOM | 9 | CE3 | TRP | B | 490 | 21.556 | −81.974 | −13.913 | 1.00 | 57.40 | C |
| ATOM | 10 | CZ3 | TRP | B | 490 | 22.280 | −81.672 | −12.774 | 1.00 | 57.15 | C |
| ATOM | 11 | CH2 | TRP | B | 490 | 22.654 | −80.354 | −12.483 | 1.00 | 57.10 | C |
| ATOM | 12 | CZ2 | TRP | B | 490 | 22.320 | −79.316 | −13.310 | 1.00 | 57.08 | C |
| ATOM | 13 | C | TRP | B | 490 | 17.708 | −80.950 | −16.129 | 1.00 | 57.62 | C |
| ATOM | 14 | O | TRP | B | 490 | 17.365 | −80.442 | −15.050 | 1.00 | 57.65 | O |
| ATOM | 1 | N | LYS | B | 491 | 17.442 | −80.409 | −17.315 | 1.00 | 57.68 | N |
| ATOM | 2 | CA | LYS | B | 491 | 16.802 | −79.103 | −17.398 | 1.00 | 57.74 | C |
| ATOM | 3 | CB | LYS | B | 491 | 16.559 | −78.684 | −18.847 | 1.00 | 57.75 | C |
| ATOM | 4 | CG | LYS | B | 491 | 15.983 | −77.286 | −18.974 | 1.00 | 58.40 | C |
| ATOM | 5 | CD | LYS | B | 491 | 16.464 | −76.592 | −20.236 | 1.00 | 60.09 | C |
| ATOM | 6 | CE | LYS | B | 491 | 17.902 | −76.107 | −20.079 | 1.00 | 61.13 | C |
| ATOM | 7 | NZ | LYS | B | 491 | 17.986 | −75.022 | −19.050 | 1.00 | 62.38 | N |
| ATOM | 8 | C | LYS | B | 491 | 15.509 | −79.077 | −16.602 | 1.00 | 57.58 | C |
| ATOM | 9 | O | LYS | B | 491 | 15.215 | −78.090 | −15.925 | 1.00 | 57.50 | O |
| ATOM | 1 | N | LEU | B | 492 | 14.746 | −80.166 | −16.680 | 1.00 | 57.51 | N |
| ATOM | 2 | CA | LEU | B | 492 | 13.518 | −80.287 | −15.895 | 1.00 | 57.34 | C |
| ATOM | 3 | CB | LEU | B | 492 | 12.727 | −81.513 | −16.345 | 1.00 | 57.12 | C |
| ATOM | 4 | CG | LEU | B | 492 | 11.442 | −81.329 | −17.148 | 1.00 | 56.50 | C |
| ATOM | 5 | CD1 | LEU | B | 492 | 11.487 | −80.090 | −18.020 | 1.00 | 56.61 | C |
| ATOM | 6 | CD2 | LEU | B | 492 | 11.183 | −82.581 | −17.975 | 1.00 | 56.14 | C |
| ATOM | 7 | C | LEU | B | 492 | 13.778 | −80.363 | −14.383 | 1.00 | 57.59 | C |
| ATOM | 8 | O | LEU | B | 492 | 13.029 | −79.801 | −13.582 | 1.00 | 57.53 | O |
| ATOM | 1 | N | SER | B | 493 | 14.847 | −81.051 | −13.998 | 1.00 | 57.77 | N |
| ATOM | 2 | CA | SER | B | 493 | 15.079 | −81.357 | −12.595 | 1.00 | 58.08 | C |
| ATOM | 3 | CB | SER | B | 493 | 15.893 | −82.641 | −12.456 | 1.00 | 58.12 | C |
| ATOM | 4 | OG | SER | B | 493 | 15.156 | −83.746 | −12.947 | 1.00 | 58.62 | O |
| ATOM | 5 | C | SER | B | 493 | 15.738 | −80.231 | −11.812 | 1.00 | 58.20 | C |
| ATOM | 6 | O | SER | B | 493 | 15.432 | −80.036 | −10.634 | 1.00 | 58.25 | O |
| ATOM | 1 | N | ASP | B | 494 | 16.650 | −79.499 | −12.443 | 1.00 | 58.27 | N |
| ATOM | 2 | CA | ASP | B | 494 | 17.297 | −78.397 | −11.735 | 1.00 | 58.62 | C |
| ATOM | 3 | CB | ASP | B | 494 | 18.562 | −77.935 | −12.449 | 1.00 | 58.56 | C |
| ATOM | 4 | CG | ASP | B | 494 | 19.324 | −76.894 | −11.657 | 1.00 | 58.70 | C |
| ATOM | 5 | OD1 | ASP | B | 494 | 18.970 | −76.661 | −10.478 | 1.00 | 58.46 | O |
| ATOM | 6 | OD2 | ASP | B | 494 | 20.277 | −76.308 | −12.215 | 1.00 | 59.37 | O |
| ATOM | 7 | C | ASP | B | 494 | 16.321 | −77.238 | −11.581 | 1.00 | 58.87 | C |
| ATOM | 8 | O | ASP | B | 494 | 16.383 | −76.250 | −12.315 | 1.00 | 58.88 | O |
| ATOM | 1 | N | VAL | B | 495 | 15.422 | −77.368 | −10.614 | 1.00 | 59.15 | N |
| ATOM | 2 | CA | VAL | B | 495 | 14.318 | −76.438 | −10.477 | 1.00 | 59.38 | C |
| ATOM | 3 | CB | VAL | B | 495 | 13.281 | −76.921 | −9.444 | 1.00 | 59.37 | C |
| ATOM | 4 | CG1 | VAL | B | 495 | 12.186 | −75.883 | −9.265 | 1.00 | 59.25 | C |
| ATOM | 5 | CG2 | VAL | B | 495 | 12.681 | −78.234 | −9.894 | 1.00 | 59.21 | C |
| ATOM | 6 | C | VAL | B | 495 | 14.779 | −75.025 | −10.155 | 1.00 | 59.65 | C |
| ATOM | 7 | O | VAL | B | 495 | 14.327 | −74.077 | −10.794 | 1.00 | 60.04 | O |
| ATOM | 1 | N | ASP | B | 496 | 15.671 | −74.869 | −9.180 | 1.00 | 59.85 | N |
| ATOM | 2 | CA | ASP | B | 496 | 16.168 | −73.525 | −8.852 | 1.00 | 60.04 | C |
| ATOM | 3 | CB | ASP | B | 496 | 16.633 | −73.422 | −7.395 | 1.00 | 60.09 | C |
| ATOM | 4 | CG | ASP | B | 496 | 17.676 | −74.455 | −7.036 | 1.00 | 60.31 | C |
| ATOM | 5 | OD1 | ASP | B | 496 | 17.844 | −75.432 | −7.801 | 1.00 | 60.71 | O |
| ATOM | 6 | OD2 | ASP | B | 496 | 18.325 | −74.289 | −5.979 | 1.00 | 60.45 | O |
| ATOM | 7 | C | ASP | B | 496 | 17.271 | −73.111 | −9.817 | 1.00 | 60.06 | C |
| ATOM | 8 | O | ASP | B | 496 | 17.873 | −72.046 | −9.673 | 1.00 | 59.85 | O |
| ATOM | 1 | N | ARG | B | 497 | 17.499 | −73.980 | −10.802 | 1.00 | 60.35 | N |
| ATOM | 2 | CA | ARG | B | 497 | 18.462 | −73.785 | −11.891 | 1.00 | 60.65 | C |
| ATOM | 3 | CB | ARG | B | 497 | 17.785 | −73.166 | −13.129 | 1.00 | 60.76 | C |
| ATOM | 4 | CG | ARG | B | 497 | 17.801 | −71.656 | −13.234 | 1.00 | 62.10 | C |
| ATOM | 5 | CD | ARG | B | 497 | 16.567 | −71.162 | −13.975 | 1.00 | 64.37 | C |
| ATOM | 6 | NE | ARG | B | 497 | 15.450 | −70.975 | −13.051 | 1.00 | 66.05 | N |
| ATOM | 7 | CZ | ARG | B | 497 | 15.155 | −69.817 | −12.463 | 1.00 | 67.08 | C |
| ATOM | 8 | NH1 | ARG | B | 497 | 14.125 | −69.738 | −11.628 | 1.00 | 67.52 | N |
| ATOM | 9 | NH2 | ARG | B | 497 | 15.885 | −68.733 | −12.713 | 1.00 | 67.06 | N |
| ATOM | 10 | C | ARG | B | 497 | 19.771 | −73.097 | −11.481 | 1.00 | 60.58 | C |
| ATOM | 11 | O | ARG | B | 497 | 20.138 | −72.036 | −11.994 | 1.00 | 60.38 | O |
| ATOM | 1 | N | ASP | B | 498 | 20.476 | −73.740 | −10.552 | 1.00 | 60.75 | N |
| ATOM | 2 | CA | ASP | B | 498 | 21.748 | −73.227 | −10.062 | 1.00 | 60.92 | C |
| ATOM | 3 | CB | ASP | B | 498 | 21.779 | −73.207 | −8.525 | 1.00 | 60.94 | C |
| ATOM | 4 | CG | ASP | B | 498 | 21.636 | −74.597 | −7.922 | 1.00 | 60.96 | C |
| ATOM | 5 | OD1 | ASP | B | 498 | 21.077 | −75.493 | −8.593 | 1.00 | 61.07 | O |
| ATOM | 6 | OD2 | ASP | B | 498 | 22.078 | −74.792 | −6.768 | 1.00 | 60.82 | O |
| ATOM | 7 | C | ASP | B | 498 | 22.935 | −74.025 | −10.610 | 1.00 | 61.06 | C |
| ATOM | 8 | O | ASP | B | 498 | 24.084 | −73.496 | −10.694 | 1.00 | 61.39 | O |
| ATOM | 1 | N | GLY | B | 499 | 22.672 | −75.293 | −10.981 | 1.00 | 61.04 | N |
| ATOM | 2 | CA | GLY | B | 499 | 23.746 | −76.100 | −11.553 | 1.00 | 61.21 | C |
| ATOM | 3 | C | GLY | B | 499 | 24.043 | −77.366 | −10.775 | 1.00 | 61.37 | C |
| ATOM | 4 | O | GLY | B | 499 | 24.801 | −78.229 | −11.235 | 1.00 | 61.52 | O |
| ATOM | 1 | N | MET | B | 500 | 23.448 | −77.476 | −9.592 | 1.00 | 61.28 | N |
| ATOM | 2 | CA | MET | B | 500 | 23.592 | −78.669 | −8.778 | 1.00 | 61.23 | C |
| ATOM | 3 | CB | MET | B | 500 | 24.169 | −78.304 | −7.416 | 1.00 | 61.21 | C |
| ATOM | 4 | CG | MET | B | 500 | 25.572 | −77.741 | −7.473 | 1.00 | 61.10 | C |
| ATOM | 5 | SD | MET | B | 500 | 26.097 | −77.079 | −5.879 | 1.00 | 61.13 | S |
| ATOM | 6 | CE | MET | B | 500 | 27.861 | −76.890 | −6.152 | 1.00 | 61.68 | C |
| ATOM | 7 | C | MET | B | 500 | 22.237 | −79.338 | −8.606 | 1.00 | 61.33 | C |
| ATOM | 8 | O | MET | B | 500 | 21.221 | −78.816 | −9.063 | 1.00 | 61.29 | O |
| ATOM | 1 | N | LEU | B | 501 | 22.227 | −80.500 | −7.956 | 1.00 | 61.45 | N |
| ATOM | 2 | CA | LEU | B | 501 | 20.977 | −81.158 | −7.585 | 1.00 | 61.49 | C |
| ATOM | 3 | CB | LEU | B | 501 | 20.759 | −82.432 | −8.408 | 1.00 | 61.41 | C |
| ATOM | 4 | CG | LEU | B | 501 | 20.599 | −82.291 | −9.924 | 1.00 | 61.24 | C |
| ATOM | 5 | CD1 | LEU | B | 501 | 20.682 | −83.646 | −10.611 | 1.00 | 60.96 | C |
| ATOM | 6 | CD2 | LEU | B | 501 | 19.292 | −81.603 | −10.266 | 1.00 | 61.45 | C |
| ATOM | 7 | C | LEU | B | 501 | 20.980 | −81.494 | −6.095 | 1.00 | 61.59 | C |
| ATOM | 8 | O | LEU | B | 501 | 21.852 | −82.220 | −5.619 | 1.00 | 61.52 | O |
| ATOM | 1 | N | ASP | B | 502 | 20.015 | −80.952 | −5.358 | 1.00 | 61.72 | N |
| ATOM | 2 | CA | ASP | B | 502 | 19.853 | −81.329 | −3.964 | 1.00 | 61.94 | C |
| ATOM | 3 | CB | ASP | B | 502 | 19.236 | −80.192 | −3.144 | 1.00 | 62.06 | C |
| ATOM | 4 | CG | ASP | B | 502 | 17.815 | −79.857 | −3.568 | 1.00 | 62.72 | C |
| ATOM | 5 | OD1 | ASP | B | 502 | 17.039 | −80.784 | −3.890 | 1.00 | 63.46 | O |
| ATOM | 6 | OD2 | ASP | B | 502 | 17.466 | −78.655 | −3.557 | 1.00 | 63.58 | O |
| ATOM | 7 | C | ASP | B | 502 | 19.005 | −82.590 | −3.902 | 1.00 | 61.99 | C |
| ATOM | 8 | O | ASP | B | 502 | 18.595 | −83.111 | −4.937 | 1.00 | 62.07 | O |
| ATOM | 1 | N | ASP | B | 503 | 18.742 | −83.079 | −2.695 | 1.00 | 62.08 | N |
| ATOM | 2 | CA | ASP | B | 503 | 18.016 | −84.337 | −2.521 | 1.00 | 62.21 | C |
| ATOM | 3 | CB | ASP | B | 503 | 17.778 | −84.611 | −1.039 | 1.00 | 62.34 | C |
| ATOM | 4 | CG | ASP | B | 503 | 17.014 | −83.495 | −0.358 | 1.00 | 62.98 | C |
| ATOM | 5 | OD1 | ASP | B | 503 | 17.226 | −82.315 | −0.718 | 1.00 | 63.16 | O |
| ATOM | 6 | OD2 | ASP | B | 503 | 16.203 | −83.863 | 0.542 | 1.00 | 64.06 | O |
| ATOM | 7 | C | ASP | B | 503 | 16.693 | −84.407 | −3.290 | 1.00 | 62.12 | C |
| ATOM | 8 | O | ASP | B | 503 | 16.455 | −85.360 | −4.040 | 1.00 | 62.04 | O |
| ATOM | 1 | N | GLU | B | 504 | 15.837 | −83.405 | −3.104 | 1.00 | 62.02 | N |
| ATOM | 2 | CA | GLU | B | 504 | 14.531 | −83.400 | −3.757 | 1.00 | 62.12 | C |
| ATOM | 3 | CB | GLU | B | 504 | 13.687 | −82.201 | −3.309 | 1.00 | 62.02 | C |
| ATOM | 4 | CG | GLU | B | 504 | 13.660 | −81.992 | −1.794 | 1.00 | 62.68 | C |
| ATOM | 5 | CD | GLU | B | 504 | 12.496 | −81.120 | −1.327 | 1.00 | 62.81 | C |
| ATOM | 6 | OE1 | GLU | B | 504 | 12.703 | −79.905 | −1.092 | 1.00 | 63.40 | O |
| ATOM | 7 | OE2 | GLU | B | 504 | 11.370 | −81.652 | −1.189 | 1.00 | 63.95 | O |
| ATOM | 8 | C | GLU | B | 504 | 14.707 | −83.410 | −5.269 | 1.00 | 61.85 | C |
| ATOM | 9 | O | GLU | B | 504 | 14.214 | −84.308 | −5.956 | 1.00 | 61.84 | O |
| ATOM | 1 | N | GLU | B | 505 | 15.431 | −82.420 | −5.781 | 1.00 | 61.64 | N |
| ATOM | 2 | CA | GLU | B | 505 | 15.704 | −82.340 | −7.206 | 1.00 | 61.43 | C |
| ATOM | 3 | CB | GLU | B | 505 | 16.701 | −81.228 | −7.493 | 1.00 | 61.18 | C |
| ATOM | 4 | CG | GLU | B | 505 | 16.088 | −79.851 | −7.502 | 1.00 | 60.83 | C |
| ATOM | 5 | CD | GLU | B | 505 | 17.131 | −78.753 | −7.597 | 1.00 | 60.55 | C |
| ATOM | 6 | OE1 | GLU | B | 505 | 18.127 | −78.807 | −6.840 | 1.00 | 60.25 | O |
| ATOM | 7 | OE2 | GLU | B | 505 | 16.957 | −77.832 | −8.427 | 1.00 | 60.26 | O |
| ATOM | 8 | C | GLU | B | 505 | 16.235 | −83.667 | −7.734 | 1.00 | 61.67 | C |
| ATOM | 9 | O | GLU | B | 505 | 15.857 | −84.106 | −8.827 | 1.00 | 61.83 | O |
| ATOM | 1 | N | PHE | B | 506 | 17.104 | −84.309 | −6.956 | 1.00 | 61.68 | N |
| ATOM | 2 | CA | PHE | B | 506 | 17.680 | −85.586 | −7.362 | 1.00 | 61.63 | C |
| ATOM | 3 | CB | PHE | B | 506 | 18.794 | −86.020 | −6.409 | 1.00 | 61.56 | C |
| ATOM | 4 | CG | PHE | B | 506 | 19.485 | −87.290 | −6.827 | 1.00 | 61.67 | C |
| ATOM | 5 | CD1 | PHE | B | 506 | 18.970 | −88.528 | −6.469 | 1.00 | 61.62 | C |
| ATOM | 6 | CE1 | PHE | B | 506 | 19.598 | −89.695 | −6.859 | 1.00 | 61.32 | C |
| ATOM | 7 | CZ | PHE | B | 506 | 20.757 | −89.633 | −7.610 | 1.00 | 61.44 | C |
| ATOM | 8 | CE2 | PHE | B | 506 | 21.281 | −88.409 | −7.971 | 1.00 | 61.14 | C |
| ATOM | 9 | CD2 | PHE | B | 506 | 20.647 | −87.247 | −7.584 | 1.00 | 61.51 | C |
| ATOM | 10 | C | PHE | B | 506 | 16.602 | −86.658 | −7.440 | 1.00 | 61.66 | C |
| ATOM | 11 | O | PHE | B | 506 | 16.548 | −87.430 | −8.401 | 1.00 | 61.70 | O |
| ATOM | 1 | N | ALA | B | 507 | 15.745 | −86.705 | −6.425 | 1.00 | 61.76 | N |
| ATOM | 2 | CA | ALA | B | 507 | 14.624 | −87.636 | −6.436 | 1.00 | 61.66 | C |
| ATOM | 3 | CB | ALA | B | 507 | 13.794 | −87.488 | −5.177 | 1.00 | 61.65 | C |
| ATOM | 4 | C | ALA | B | 507 | 13.768 | −87.405 | −7.679 | 1.00 | 61.68 | C |
| ATOM | 5 | O | ALA | B | 507 | 13.344 | −88.361 | −8.332 | 1.00 | 61.70 | O |
| ATOM | 1 | N | LEU | B | 508 | 13.531 | −86.133 | −8.005 | 1.00 | 61.60 | N |
| ATOM | 2 | CA | LEU | B | 508 | 12.805 | −85.767 | −9.222 | 1.00 | 61.54 | C |
| ATOM | 3 | CB | LEU | B | 508 | 12.722 | −84.247 | −9.353 | 1.00 | 61.44 | C |
| ATOM | 4 | CG | LEU | B | 508 | 11.482 | −83.623 | −9.997 | 1.00 | 61.08 | C |
| ATOM | 5 | CD1 | LEU | B | 508 | 11.788 | −82.195 | −10.402 | 1.00 | 60.85 | C |
| ATOM | 6 | CD2 | LEU | B | 508 | 10.995 | −84.417 | −11.192 | 1.00 | 60.65 | C |
| ATOM | 7 | C | LEU | B | 508 | 13.509 | −86.346 | −10.450 | 1.00 | 61.70 | C |
| ATOM | 8 | O | LEU | B | 508 | 12.905 | −87.063 | −11.262 | 1.00 | 61.61 | O |
| ATOM | 1 | N | ALA | B | 509 | 14.792 | −86.026 | −10.582 | 1.00 | 61.77 | N |
| ATOM | 2 | CA | ALA | B | 509 | 15.593 | −86.572 | −11.662 | 1.00 | 61.78 | C |
| ATOM | 3 | CB | ALA | B | 509 | 17.060 | −86.243 | −11.449 | 1.00 | 61.77 | C |
| ATOM | 4 | C | ALA | B | 509 | 15.378 | −88.082 | −11.768 | 1.00 | 61.87 | C |
| ATOM | 5 | O | ALA | B | 509 | 14.950 | −88.585 | −12.810 | 1.00 | 61.97 | O |
| ATOM | 1 | N | SER | B | 510 | 15.654 | −88.798 | −10.681 | 1.00 | 61.83 | N |
| ATOM | 2 | CA | SER | B | 510 | 15.496 | −90.251 | −10.657 | 1.00 | 61.95 | C |
| ATOM | 3 | CB | SER | B | 510 | 15.749 | −90.793 | −9.249 | 1.00 | 62.00 | C |
| ATOM | 4 | OG | SER | B | 510 | 16.993 | −90.333 | −8.748 | 1.00 | 62.65 | O |
| ATOM | 5 | C | SER | B | 510 | 14.110 | −90.673 | −11.135 | 1.00 | 61.79 | C |
| ATOM | 6 | O | SER | B | 510 | 13.978 | −91.562 | −11.987 | 1.00 | 61.78 | O |
| ATOM | 1 | N | HIS | B | 511 | 13.084 | −90.030 | −10.579 | 1.00 | 61.62 | N |
| ATOM | 2 | CA | HIS | B | 511 | 11.707 | −90.302 | −10.970 | 1.00 | 61.40 | C |
| ATOM | 3 | CB | HIS | B | 511 | 10.742 | −89.312 | −10.307 | 1.00 | 61.24 | C |
| ATOM | 4 | CG | HIS | B | 511 | 9.317 | −89.470 | −10.744 | 1.00 | 60.84 | C |
| ATOM | 5 | ND1 | HIS | B | 511 | 8.390 | −90.185 | −10.016 | 1.00 | 60.78 | N |
| ATOM | 6 | CE1 | HIS | B | 511 | 7.225 | −90.155 | −10.639 | 1.00 | 60.24 | C |
| ATOM | 7 | NE2 | HIS | B | 511 | 7.363 | −89.449 | −11.747 | 1.00 | 60.07 | N |
| ATOM | 8 | CD2 | HIS | B | 511 | 8.662 | −89.009 | −11.836 | 1.00 | 60.36 | C |
| ATOM | 9 | C | HIS | B | 511 | 11.594 | −90.212 | −12.479 | 1.00 | 61.38 | C |
| ATOM | 10 | O | HIS | B | 511 | 11.092 | −91.124 | −13.132 | 1.00 | 61.56 | O |
| ATOM | 1 | N | LEU | B | 512 | 12.083 | −89.109 | −13.031 | 1.00 | 61.36 | N |
| ATOM | 2 | CA | LEU | B | 512 | 12.001 | −88.881 | −14.464 | 1.00 | 61.37 | C |
| ATOM | 3 | CB | LEU | B | 512 | 12.489 | −87.469 | −14.786 | 1.00 | 61.22 | C |
| ATOM | 4 | CG | LEU | B | 512 | 11.466 | −86.435 | −14.310 | 1.00 | 60.86 | C |
| ATOM | 5 | CD1 | LEU | B | 512 | 12.018 | −85.018 | −14.367 | 1.00 | 60.51 | C |
| ATOM | 6 | CD2 | LEU | B | 512 | 10.185 | −86.565 | −15.123 | 1.00 | 59.84 | C |
| ATOM | 7 | C | LEU | B | 512 | 12.751 | −89.938 | −15.278 | 1.00 | 61.48 | C |
| ATOM | 8 | O | LEU | B | 512 | 12.264 | −90.422 | −16.309 | 1.00 | 61.48 | O |
| ATOM | 1 | N | ILE | B | 513 | 13.929 | −90.309 | −14.793 | 1.00 | 61.74 | N |
| ATOM | 2 | CA | ILE | B | 513 | 14.709 | −91.371 | −15.415 | 1.00 | 61.83 | C |
| ATOM | 3 | CB | ILE | B | 513 | 16.079 | −91.518 | −14.728 | 1.00 | 61.81 | C |
| ATOM | 4 | CG1 | ILE | B | 513 | 16.825 | −90.181 | −14.799 | 1.00 | 61.73 | C |
| ATOM | 5 | CD1 | ILE | B | 513 | 18.282 | −90.257 | −14.427 | 1.00 | 62.12 | C |
| ATOM | 6 | CG2 | ILE | B | 513 | 16.882 | −92.645 | −15.365 | 1.00 | 61.68 | C |
| ATOM | 7 | C | ILE | B | 513 | 13.946 | −92.705 | −15.439 | 1.00 | 61.89 | C |
| ATOM | 8 | O | ILE | B | 513 | 13.817 | −93.333 | −16.492 | 1.00 | 61.85 | O |
| ATOM | 1 | N | GLU | B | 514 | 13.429 | −93.124 | −14.285 | 1.00 | 61.92 | N |
| ATOM | 2 | CA | GLU | B | 514 | 12.589 | −94.317 | −14.223 | 1.00 | 62.03 | C |
| ATOM | 3 | CB | GLU | B | 514 | 12.044 | −94.519 | −12.814 | 1.00 | 62.08 | C |
| ATOM | 4 | CG | GLU | B | 514 | 13.104 | −94.778 | −11.765 | 1.00 | 62.83 | C |
| ATOM | 5 | CD | GLU | B | 514 | 12.556 | −95.511 | −10.550 | 1.00 | 63.54 | C |
| ATOM | 6 | OE1 | GLU | B | 514 | 11.502 | −96.181 | −10.667 | 1.00 | 63.39 | O |
| ATOM | 7 | OE2 | GLU | B | 514 | 13.190 | −95.420 | −9.476 | 1.00 | 64.11 | O |
| ATOM | 8 | C | GLU | B | 514 | 11.423 | −94.201 | −15.194 | 1.00 | 62.07 | C |
| ATOM | 9 | O | GLU | B | 514 | 11.155 | −95.108 | −15.989 | 1.00 | 62.33 | O |
| ATOM | 1 | N | ALA | B | 515 | 10.725 | −93.072 | −15.116 | 1.00 | 61.99 | N |
| ATOM | 2 | CA | ALA | B | 515 | 9.619 | −92.794 | −16.018 | 1.00 | 61.95 | C |
| ATOM | 3 | CB | ALA | B | 515 | 9.162 | −91.346 | −15.876 | 1.00 | 61.86 | C |
| ATOM | 4 | C | ALA | B | 515 | 10.023 | −93.091 | −17.457 | 1.00 | 61.91 | C |
| ATOM | 5 | O | ALA | B | 515 | 9.366 | −93.878 | −18.137 | 1.00 | 61.89 | O |
| ATOM | 1 | N | LYS | B | 516 | 11.111 | −92.472 | −17.911 | 1.00 | 61.87 | N |
| ATOM | 2 | CA | LYS | B | 516 | 11.554 | −92.658 | −19.291 | 1.00 | 61.79 | C |
| ATOM | 3 | CB | LYS | B | 516 | 12.747 | −91.760 | −19.625 | 1.00 | 61.68 | C |
| ATOM | 4 | CG | LYS | B | 516 | 12.626 | −91.054 | −20.968 | 1.00 | 61.28 | C |
| ATOM | 5 | CD | LYS | B | 516 | 12.641 | −92.024 | −22.133 | 1.00 | 61.20 | C |
| ATOM | 6 | CE | LYS | B | 516 | 11.698 | −91.567 | −23.236 | 1.00 | 61.34 | C |
| ATOM | 7 | NZ | LYS | B | 516 | 11.813 | −90.108 | −23.506 | 1.00 | 61.19 | N |
| ATOM | 8 | C | LYS | B | 516 | 11.902 | −94.113 | −19.547 | 1.00 | 61.88 | C |
| ATOM | 9 | O | LYS | B | 516 | 11.602 | −94.645 | −20.614 | 1.00 | 62.04 | O |
| ATOM | 1 | N | LEU | B | 517 | 12.525 | −94.761 | −18.568 | 1.00 | 61.97 | N |
| ATOM | 2 | CA | LEU | B | 517 | 12.857 | −96.176 | −18.705 | 1.00 | 62.25 | C |
| ATOM | 3 | CB | LEU | B | 517 | 13.632 | −96.690 | −17.487 | 1.00 | 62.24 | C |
| ATOM | 4 | CG | LEU | B | 517 | 15.064 | −96.164 | −17.332 | 1.00 | 62.30 | C |
| ATOM | 5 | CD1 | LEU | B | 517 | 15.725 | −96.729 | −16.083 | 1.00 | 62.24 | C |
| ATOM | 6 | CD2 | LEU | B | 517 | 15.895 | −96.473 | −18.570 | 1.00 | 62.23 | C |
| ATOM | 7 | C | LEU | B | 517 | 11.607 | −97.019 | −18.948 | 1.00 | 62.46 | C |
| ATOM | 8 | O | LEU | B | 517 | 11.629 | −97.939 | −19.760 | 1.00 | 62.42 | O |
| ATOM | 1 | N | GLU | B | 518 | 10.517 | −96.698 | −18.255 | 1.00 | 62.79 | N |
| ATOM | 2 | CA | GLU | B | 518 | 9.248 | −97.404 | −18.465 | 1.00 | 63.31 | C |
| ATOM | 3 | CB | GLU | B | 518 | 8.186 | −96.944 | −17.462 | 1.00 | 63.45 | C |
| ATOM | 4 | CG | GLU | B | 518 | 7.762 | −98.010 | −16.464 | 1.00 | 63.87 | C |
| ATOM | 5 | CD | GLU | B | 518 | 8.718 | −98.127 | −15.302 | 1.00 | 64.37 | C |
| ATOM | 6 | OE1 | GLU | B | 518 | 9.452 | −99.135 | −15.238 | 1.00 | 64.42 | O |
| ATOM | 7 | OE2 | GLU | B | 518 | 8.741 | −97.205 | −14.457 | 1.00 | 64.82 | O |
| ATOM | 8 | C | GLU | B | 518 | 8.701 | −97.252 | −19.882 | 1.00 | 63.54 | C |
| ATOM | 9 | O | GLU | B | 518 | 7.929 | −98.091 | −20.351 | 1.00 | 63.50 | O |
| ATOM | 1 | N | GLY | B | 519 | 9.094 | −96.175 | −20.554 | 1.00 | 63.92 | N |
| ATOM | 2 | CA | GLY | B | 519 | 8.584 | −95.872 | −21.888 | 1.00 | 64.42 | C |
| ATOM | 3 | C | GLY | B | 519 | 7.591 | −94.725 | −21.875 | 1.00 | 64.74 | C |
| ATOM | 4 | O | GLY | B | 519 | 6.488 | −94.834 | −22.415 | 1.00 | 64.74 | O |
| ATOM | 1 | N | HIS | B | 520 | 7.987 | −93.620 | −21.250 | 1.00 | 65.16 | N |
| ATOM | 2 | CA | HIS | B | 520 | 7.139 | −92.438 | −21.167 | 1.00 | 65.57 | C |
| ATOM | 3 | CB | HIS | B | 520 | 6.765 | −92.143 | −19.714 | 1.00 | 65.67 | C |
| ATOM | 4 | CG | HIS | B | 520 | 5.660 | −93.003 | −19.192 | 1.00 | 66.10 | C |
| ATOM | 5 | ND1 | HIS | B | 520 | 4.337 | −92.796 | −19.526 | 1.00 | 66.58 | N |
| ATOM | 6 | CE1 | HIS | B | 520 | 3.589 | −93.707 | −18.928 | 1.00 | 66.72 | C |
| ATOM | 7 | NE2 | HIS | B | 520 | 4.379 | −94.495 | −18.217 | 1.00 | 66.89 | N |
| ATOM | 8 | CD2 | HIS | B | 520 | 5.680 | −94.077 | −18.366 | 1.00 | 66.36 | C |
| ATOM | 9 | C | HIS | B | 520 | 7.822 | −91.219 | −21.757 | 1.00 | 65.70 | C |
| ATOM | 10 | O | HIS | B | 520 | 8.924 | −90.855 | −21.345 | 1.00 | 65.77 | O |
| ATOM | 1 | N | GLY | B | 521 | 7.165 | −90.583 | −22.720 | 1.00 | 65.86 | N |
| ATOM | 2 | CA | GLY | B | 521 | 7.633 | −89.296 | −23.205 | 1.00 | 66.01 | C |
| ATOM | 3 | C | GLY | B | 521 | 7.785 | −88.376 | −22.010 | 1.00 | 66.07 | C |
| ATOM | 4 | O | GLY | B | 521 | 6.976 | −88.416 | −21.081 | 1.00 | 66.13 | O |
| ATOM | 1 | N | LEU | B | 522 | 8.837 | −87.569 | −22.009 | 1.00 | 66.10 | N |
| ATOM | 2 | CA | LEU | B | 522 | 9.017 | −86.582 | −20.954 | 1.00 | 66.04 | C |
| ATOM | 3 | CB | LEU | B | 522 | 10.481 | −86.174 | −20.840 | 1.00 | 66.08 | C |
| ATOM | 4 | CG | LEU | B | 522 | 11.321 | −86.795 | −19.726 | 1.00 | 66.47 | C |
| ATOM | 5 | CD1 | LEU | B | 522 | 10.858 | −88.202 | −19.364 | 1.00 | 66.89 | C |
| ATOM | 6 | CD2 | LEU | B | 522 | 12.776 | −86.798 | −20.163 | 1.00 | 66.94 | C |
| ATOM | 7 | C | LEU | B | 522 | 8.178 | −85.351 | −21.244 | 1.00 | 65.97 | C |
| ATOM | 8 | O | LEU | B | 522 | 7.966 | −84.999 | −22.405 | 1.00 | 65.87 | O |
| ATOM | 1 | N | PRO | B | 523 | 7.692 | −84.690 | −20.184 | 1.00 | 65.92 | N |
| ATOM | 2 | CA | PRO | B | 523 | 7.027 | −83.409 | −20.367 | 1.00 | 65.84 | C |
| ATOM | 3 | CB | PRO | B | 523 | 6.651 | −82.993 | −18.940 | 1.00 | 65.78 | C |
| ATOM | 4 | CG | PRO | B | 523 | 6.708 | −84.241 | −18.133 | 1.00 | 65.95 | C |
| ATOM | 5 | CD | PRO | B | 523 | 7.749 | −85.097 | −18.770 | 1.00 | 65.96 | C |
| ATOM | 6 | C | PRO | B | 523 | 8.024 | −82.414 | −20.944 | 1.00 | 65.77 | C |
| ATOM | 7 | O | PRO | B | 523 | 9.228 | −82.685 | −20.982 | 1.00 | 65.97 | O |
| ATOM | 1 | N | THR | B | 524 | 7.535 | −81.272 | −21.399 | 1.00 | 65.48 | N |
| ATOM | 2 | CA | THR | B | 524 | 8.435 | −80.238 | −21.863 | 1.00 | 65.17 | C |
| ATOM | 3 | CB | THR | B | 524 | 8.019 | −79.717 | −23.260 | 1.00 | 65.20 | C |
| ATOM | 4 | OG1 | THR | B | 524 | 8.323 | −78.323 | −23.369 | 1.00 | 65.65 | O |
| ATOM | 5 | CG2 | THR | B | 524 | 6.526 | −79.937 | −23.494 | 1.00 | 65.23 | O |
| ATOM | 6 | C | THR | B | 524 | 8.548 | −79.137 | −20.799 | 1.00 | 64.84 | C |
| ATOM | 7 | O | THR | B | 524 | 9.526 | −78.392 | −20.761 | 1.00 | 64.93 | O |
| ATOM | 1 | N | ASN | B | 525 | 7.554 | −79.070 | −19.917 | 1.00 | 64.41 | N |
| ATOM | 2 | CA | ASN | B | 525 | 7.559 | −78.137 | −18.788 | 1.00 | 63.87 | C |
| ATOM | 3 | CB | ASN | B | 525 | 6.682 | −76.920 | −19.078 | 1.00 | 63.88 | C |
| ATOM | 4 | CG | ASN | B | 525 | 7.333 | −75.948 | −20.029 | 1.00 | 63.78 | C |
| ATOM | 5 | OD1 | ASN | B | 525 | 6.763 | −75.603 | −21.063 | 1.00 | 63.84 | O |
| ATOM | 6 | ND2 | ASN | B | 525 | 8.539 | −75.502 | −19.689 | 1.00 | 63.55 | N |
| ATOM | 7 | C | ASN | B | 525 | 7.078 | −78.805 | −17.509 | 1.00 | 63.51 | C |
| ATOM | 8 | O | ASN | B | 525 | 5.970 | −79.340 | −17.455 | 1.00 | 63.55 | O |
| ATOM | 1 | N | LEU | B | 526 | 7.917 | −78.761 | −16.482 | 1.00 | 62.99 | N |
| ATOM | 2 | CA | LEU | B | 526 | 7.618 | −79.378 | −15.202 | 1.00 | 62.43 | C |
| ATOM | 3 | CB | LEU | B | 526 | 8.806 | −79.165 | −14.268 | 1.00 | 62.46 | C |
| ATOM | 4 | CG | LEU | B | 526 | 9.131 | −80.239 | −13.233 | 1.00 | 62.26 | C |
| ATOM | 5 | CD1 | LEU | B | 526 | 8.105 | −80.225 | −12.130 | 1.00 | 62.66 | C |
| ATOM | 6 | CD2 | LEU | B | 526 | 9.197 | −81.602 | −13.887 | 1.00 | 62.13 | C |
| ATOM | 7 | C | LEU | B | 526 | 6.354 | −78.766 | −14.597 | 1.00 | 62.24 | C |
| ATOM | 8 | O | LEU | B | 526 | 6.355 | −77.599 | −14.209 | 1.00 | 62.21 | O |
| ATOM | 1 | N | PRO | B | 527 | 5.266 | −79.554 | −14.512 | 1.00 | 61.99 | N |
| ATOM | 2 | CA | PRO | B | 527 | 4.014 | −79.062 | −13.941 | 1.00 | 61.70 | C |
| ATOM | 3 | CB | PRO | B | 527 | 2.997 | −80.112 | −14.388 | 1.00 | 61.70 | C |
| ATOM | 4 | CG | PRO | B | 527 | 3.778 | −81.364 | −14.488 | 1.00 | 61.61 | C |
| ATOM | 5 | CD | PRO | B | 527 | 5.160 | −80.961 | −14.942 | 1.00 | 61.99 | C |
| ATOM | 6 | C | PRO | B | 527 | 4.083 | −79.005 | −12.417 | 1.00 | 61.51 | C |
| ATOM | 7 | O | PRO | B | 527 | 4.849 | −79.751 | −11.804 | 1.00 | 61.49 | O |
| ATOM | 1 | N | ARG | B | 528 | 3.279 | −78.133 | −11.814 | 1.00 | 61.27 | N |
| ATOM | 2 | CA | ARG | B | 528 | 3.346 | −77.888 | −10.373 | 1.00 | 61.10 | C |
| ATOM | 3 | CB | ARG | B | 528 | 2.166 | −77.024 | −9.915 | 1.00 | 61.10 | C |
| ATOM | 4 | CG | ARG | B | 528 | 2.137 | −76.776 | −8.411 | 1.00 | 61.06 | C |
| ATOM | 5 | CD | ARG | B | 528 | 0.879 | −76.043 | −7.980 | 1.00 | 61.02 | C |
| ATOM | 6 | NE | ARG | B | 528 | 0.850 | −75.823 | −6.536 | 1.00 | 60.86 | N |
| ATOM | 7 | CZ | ARG | B | 528 | 1.218 | −74.691 | −5.938 | 1.00 | 60.65 | C |
| ATOM | 8 | NH1 | ARG | B | 528 | 1.157 | −74.592 | −4.616 | 1.00 | 60.39 | N |
| ATOM | 9 | NH2 | ARG | B | 528 | 1.645 | −73.658 | −6.656 | 1.00 | 60.55 | N |
| ATOM | 10 | C | ARG | B | 528 | 3.409 | −79.163 | −9.528 | 1.00 | 61.02 | C |
| ATOM | 11 | O | ARG | B | 528 | 4.218 | −79.270 | −8.606 | 1.00 | 60.96 | O |
| ATOM | 1 | N | ARG | B | 529 | 2.552 | −80.125 | −9.851 | 1.00 | 60.92 | N |
| ATOM | 2 | CA | ARG | B | 529 | 2.403 | −81.325 | −9.035 | 1.00 | 60.87 | C |
| ATOM | 3 | CB | ARG | B | 529 | 1.251 | −82.189 | −9.555 | 1.00 | 61.05 | C |
| ATOM | 4 | CG | ARG | B | 529 | 1.382 | −82.565 | −11.016 | 1.00 | 61.92 | C |
| ATOM | 5 | CD | ARG | B | 529 | 0.162 | −83.315 | −11.516 | 1.00 | 63.40 | C |
| ATOM | 6 | NE | ARG | B | 529 | 0.370 | −83.798 | −12.877 | 1.00 | 64.56 | N |
| ATOM | 7 | CZ | ARG | B | 529 | 1.047 | −84.901 | −13.181 | 1.00 | 65.44 | C |
| ATOM | 8 | NH1 | ARG | B | 529 | 1.189 | −85.259 | −14.451 | 1.00 | 65.89 | N |
| ATOM | 9 | NH2 | ARG | B | 529 | 1.584 | −85.646 | −12.218 | 1.00 | 65.76 | N |
| ATOM | 10 | C | ARG | B | 529 | 3.683 | −82.142 | −8.958 | 1.00 | 60.59 | C |
| ATOM | 11 | O | ARG | B | 529 | 3.835 | −82.979 | −8.066 | 1.00 | 60.74 | O |
| ATOM | 1 | N | LEU | B | 530 | 4.599 | −81.907 | −9.892 | 1.00 | 60.16 | N |
| ATOM | 2 | CA | LEU | B | 530 | 5.877 | −82.605 | −9.880 | 1.00 | 59.71 | C |
| ATOM | 3 | CB | LEU | B | 530 | 6.291 | −83.023 | −11.292 | 1.00 | 59.65 | C |
| ATOM | 4 | CG | LEU | B | 530 | 5.799 | −84.382 | −11.791 | 1.00 | 59.14 | C |
| ATOM | 5 | CD1 | LEU | B | 530 | 6.397 | −84.693 | −13.153 | 1.00 | 58.59 | C |
| ATOM | 6 | CD2 | LEU | B | 530 | 6.155 | −85.472 | −10.801 | 1.00 | 58.67 | C |
| ATOM | 7 | C | LEU | B | 530 | 6.980 | −81.784 | −9.219 | 1.00 | 59.56 | C |
| ATOM | 8 | O | LEU | B | 530 | 8.032 | −82.318 | −8.874 | 1.00 | 59.51 | O |
| ATOM | 1 | N | VAL | B | 531 | 6.744 | −80.488 | −9.043 | 1.00 | 59.45 | N |
| ATOM | 2 | CA | VAL | B | 531 | 7.716 | −79.637 | −8.364 | 1.00 | 59.44 | C |
| ATOM | 3 | CB | VAL | B | 531 | 7.333 | −78.142 | −8.445 | 1.00 | 59.28 | C |
| ATOM | 4 | CG1 | VAL | B | 531 | 8.433 | −77.283 | −7.859 | 1.00 | 58.89 | C |
| ATOM | 5 | CG2 | VAL | B | 531 | 7.057 | −77.735 | −9.877 | 1.00 | 59.32 | C |
| ATOM | 6 | C | VAL | B | 531 | 7.815 | −80.033 | −6.894 | 1.00 | 59.61 | C |
| ATOM | 7 | O | VAL | B | 531 | 6.800 | −80.081 | −6.198 | 1.00 | 59.64 | O |
| ATOM | 1 | N | PRO | B | 532 | 9.036 | −80.329 | −6.416 | 1.00 | 59.71 | N |
| ATOM | 2 | CA | PRO | B | 532 | 9.218 | −80.580 | −4.992 | 1.00 | 59.85 | C |
| ATOM | 3 | CB | PRO | B | 532 | 10.724 | −80.410 | −4.804 | 1.00 | 59.82 | C |
| ATOM | 4 | CG | PRO | B | 532 | 11.307 | −80.816 | −6.117 | 1.00 | 59.65 | C |
| ATOM | 5 | CD | PRO | B | 532 | 10.297 | −80.447 | −7.170 | 1.00 | 59.60 | C |
| ATOM | 6 | C | PRO | B | 532 | 8.456 | −79.537 | −4.176 | 1.00 | 60.13 | C |
| ATOM | 7 | O | PRO | B | 532 | 8.415 | −78.370 | −4.561 | 1.00 | 60.15 | O |
| ATOM | 1 | N | PRO | B | 533 | 7.852 | −79.960 | −3.054 | 1.00 | 60.38 | N |
| ATOM | 2 | CA | PRO | B | 533 | 6.965 | −79.145 | −2.224 | 1.00 | 60.52 | C |
| ATOM | 3 | CB | PRO | B | 533 | 6.841 | −79.971 | −0.945 | 1.00 | 60.55 | C |
| ATOM | 4 | CG | PRO | B | 533 | 6.957 | −81.366 | −1.425 | 1.00 | 60.51 | C |
| ATOM | 5 | CD | PRO | B | 533 | 8.002 | −81.321 | −2.510 | 1.00 | 60.41 | C |
| ATOM | 6 | C | PRO | B | 533 | 7.469 | −77.729 | −1.918 | 1.00 | 60.72 | C |
| ATOM | 7 | O | PRO | B | 533 | 6.658 | −76.825 | −1.727 | 1.00 | 60.82 | O |
| ATOM | 1 | N | SER | B | 534 | 8.783 | −77.532 | −1.861 | 1.00 | 61.01 | N |
| ATOM | 2 | CA | SER | B | 534 | 9.324 | −76.176 | −1.855 | 1.00 | 61.33 | C |
| ATOM | 3 | CB | SER | B | 534 | 10.781 | −76.179 | −1.414 | 1.00 | 61.27 | C |
| ATOM | 4 | OG | SER | B | 534 | 10.965 | −77.089 | −0.342 | 1.00 | 61.06 | O |
| ATOM | 5 | C | SER | B | 534 | 9.180 | −75.634 | −3.277 | 1.00 | 61.73 | C |
| ATOM | 6 | O | SER | B | 534 | 10.158 | −75.532 | −4.034 | 1.00 | 61.49 | O |
| ATOM | 1 | N | LYS | B | 535 | 7.939 | −75.302 | −3.635 | 1.00 | 62.26 | N |
| ATOM | 2 | CA | LYS | B | 535 | 7.591 | −75.061 | −5.034 | 1.00 | 62.66 | C |
| ATOM | 3 | CB | LYS | B | 535 | 6.481 | −76.020 | −5.521 | 1.00 | 62.74 | C |
| ATOM | 4 | CG | LYS | B | 535 | 5.237 | −76.121 | −4.644 | 1.00 | 62.63 | C |
| ATOM | 5 | CD | LYS | B | 535 | 4.224 | −77.127 | −5.215 | 1.00 | 62.53 | C |
| ATOM | 6 | CE | LYS | B | 535 | 4.611 | −78.574 | −4.909 | 1.00 | 62.10 | C |
| ATOM | 7 | NZ | LYS | B | 535 | 3.545 | −79.555 | −5.271 | 1.00 | 61.50 | N |
| ATOM | 8 | C | LYS | B | 535 | 7.279 | −73.624 | −5.416 | 1.00 | 62.89 | C |
| ATOM | 9 | O | LYS | B | 535 | 8.077 | −73.007 | −6.120 | 1.00 | 63.20 | O |
| ATOM | 1 | N | ARG | B | 536 | 6.144 | −73.086 | −4.968 | 1.00 | 62.87 | N |
| ATOM | 2 | CA | ARG | B | 536 | 5.680 | −71.790 | −5.472 | 1.00 | 62.96 | C |
| ATOM | 3 | CB | ARG | B | 536 | 5.145 | −71.935 | −6.902 | 1.00 | 63.00 | C |
| ATOM | 4 | CG | ARG | B | 536 | 5.911 | −71.160 | −7.963 | 1.00 | 62.88 | C |
| ATOM | 5 | CD | ARG | B | 536 | 5.396 | −69.730 | −8.098 | 1.00 | 62.78 | C |
| ATOM | 6 | NE | ARG | B | 536 | 5.987 | −69.039 | −9.246 | 1.00 | 62.80 | N |
| ATOM | 7 | CZ | ARG | B | 536 | 5.838 | −67.741 | −9.505 | 1.00 | 62.71 | C |
| ATOM | 8 | NH1 | ARG | B | 536 | 6.415 | −67.202 | −10.571 | 1.00 | 62.64 | N |
| ATOM | 9 | NH2 | ARG | B | 536 | 5.115 | −66.977 | −8.697 | 1.00 | 62.73 | N |
| ATOM | 10 | C | ARG | B | 536 | 4.593 | −71.172 | −4.622 | 1.00 | 63.09 | C |
| ATOM | 11 | O | ARG | B | 536 | 3.436 | −71.595 | −4.688 | 1.00 | 63.09 | O |
| ATOM | 1 | N | ARG | B | 537 | 4.964 | −70.154 | −3.843 | 1.00 | 63.37 | N |
| ATOM | 2 | CA | ARG | B | 537 | 3.993 | −69.382 | −3.075 | 1.00 | 63.60 | C |
| ATOM | 3 | CB | ARG | B | 537 | 4.697 | −68.242 | −2.327 | 1.00 | 63.66 | C |
| ATOM | 4 | CG | ARG | B | 537 | 4.130 | −67.921 | −0.934 | 1.00 | 64.02 | C |
| ATOM | 5 | CD | ARG | B | 537 | 2.685 | −67.398 | −0.982 | 1.00 | 64.23 | C |
| ATOM | 6 | NE | ARG | B | 537 | 2.224 | −66.952 | 0.333 | 1.00 | 64.87 | N |
| ATOM | 7 | CZ | ARG | B | 537 | 1.914 | −65.692 | 0.633 | 1.00 | 65.30 | C |
| ATOM | 8 | NH1 | ARG | B | 537 | 2.007 | −64.746 | −0.292 | 1.00 | 65.61 | N |
| ATOM | 9 | NH2 | ARG | B | 537 | 1.502 | −65.373 | 1.856 | 1.00 | 64.97 | N |
| ATOM | 10 | C | ARG | B | 537 | 2.970 | −68.816 | −4.063 | 1.00 | 63.69 | C |
| ATOM | 11 | O | ARG | B | 537 | 3.298 | −68.553 | −5.226 | 1.00 | 63.76 | O |
| ATOM | 1 | N | GLN | B | 538 | 1.734 | −68.641 | −3.608 | 1.00 | 63.75 | N |
| ATOM | 2 | CA | GLN | B | 538 | 0.666 | −68.177 | −4.489 | 1.00 | 63.87 | C |
| ATOM | 3 | CB | GLN | B | 538 | 0.043 | −69.363 | −5.225 | 1.00 | 63.84 | C |
| ATOM | 4 | CG | GLN | B | 538 | −0.406 | −70.488 | −4.301 | 1.00 | 63.92 | C |
| ATOM | 5 | CD | GLN | B | 538 | −0.796 | −71.738 | −5.066 | 1.00 | 64.01 | C |
| ATOM | 6 | OE1 | GLN | B | 538 | −0.803 | −71.751 | −6.299 | 1.00 | 64.15 | O |
| ATOM | 7 | NE2 | GLN | B | 538 | −1.122 | −72.802 | −4.338 | 1.00 | 64.37 | N |
| ATOM | 8 | C | GLN | B | 538 | −0.409 | −67.416 | −3.723 | 1.00 | 63.88 | C |
| ATOM | 9 | O | GLN | B | 538 | −1.543 | −67.243 | −4.236 | 1.00 | 63.92 | O |
| TER | 9 | | GLN | B | 538 | | | | | | |
| HETATM | 1 | O2A | ANP | B | 600 | 3.973 | −56.574 | −52.078 | 1.00 | 48.61 | O |
| HETATM | 2 | PA | ANP | B | 600 | 4.052 | −57.106 | −50.670 | 1.00 | 48.08 | P |
| HETATM | 3 | O1A | ANP | B | 600 | 2.910 | −57.937 | −50.141 | 1.00 | 48.41 | O |
| HETATM | 4 | O3A | ANP | B | 600 | 4.279 | −55.854 | −49.694 | 1.00 | 49.01 | O |
| HETATM | 5 | PB | ANP | B | 600 | 3.622 | −54.443 | −50.096 | 1.00 | 49.55 | P |
| HETATM | 6 | O1B | ANP | B | 600 | 3.428 | −53.654 | −48.825 | 1.00 | 49.44 | O |
| HETATM | 7 | O2B | ANP | B | 600 | 2.412 | −54.721 | −50.957 | 1.00 | 49.32 | O |
| HETATM | 8 | N3B | ANP | B | 600 | 4.812 | −53.641 | −51.080 | 1.00 | 49.69 | N |
| HETATM | 9 | PG | ANP | B | 600 | 4.339 | −52.172 | −51.877 | 1.00 | 50.29 | P |
| HETATM | 10 | O3G | ANP | B | 600 | 4.467 | −51.114 | −50.807 | 1.00 | 50.68 | O |
| HETATM | 11 | O2G | ANP | B | 600 | 5.350 | −52.031 | −52.990 | 1.00 | 50.99 | O |
| HETATM | 12 | O1G | ANP | B | 600 | 2.921 | −52.435 | −52.332 | 1.00 | 49.88 | O |
| HETATM | 13 | O5* | ANP | B | 600 | 5.406 | −57.950 | −50.510 | 1.00 | 48.33 | O |
| HETATM | 14 | C5* | ANP | B | 600 | 6.435 | −57.592 | −49.597 | 1.00 | 49.10 | C |
| HETATM | 15 | C4* | ANP | B | 600 | 7.544 | −58.626 | −49.749 | 1.00 | 50.21 | C |
| HETATM | 16 | C3* | ANP | B | 600 | 7.191 | −59.661 | −50.803 | 1.00 | 50.31 | C |
| HETATM | 17 | O3* | ANP | B | 600 | 8.395 | −60.157 | −51.392 | 1.00 | 50.26 | O |
| HETATM | 18 | C2* | ANP | B | 600 | 6.537 | −60.782 | −50.030 | 1.00 | 50.21 | C |
| HETATM | 19 | O2* | ANP | B | 600 | 6.795 | −62.044 | −50.644 | 1.00 | 50.10 | O |
| HETATM | 20 | C1* | ANP | B | 600 | 7.209 | −60.692 | −48.675 | 1.00 | 50.66 | C |
| HETATM | 21 | O4* | ANP | B | 600 | 7.693 | −59.354 | −48.530 | 1.00 | 50.80 | O |
| HETATM | 22 | N9 | ANP | B | 600 | 6.247 | −60.938 | −47.587 | 1.00 | 50.73 | N |
| HETATM | 23 | C4 | ANP | B | 600 | 6.554 | −61.536 | −46.440 | 1.00 | 50.52 | C |
| HETATM | 24 | C5 | ANP | B | 600 | 5.320 | −61.565 | −45.655 | 1.00 | 50.67 | C |
| HETATM | 25 | N7 | ANP | B | 600 | 4.370 | −60.971 | −46.403 | 1.00 | 51.15 | N |
| HETATM | 26 | C8 | ANP | B | 600 | 4.948 | −60.592 | −47.574 | 1.00 | 50.79 | C |
| HETATM | 27 | N3 | ANP | B | 600 | 7.677 | −62.071 | −45.921 | 1.00 | 50.46 | N |
| HETATM | 28 | C2 | ANP | B | 600 | 7.672 | −62.625 | −44.699 | 1.00 | 50.75 | C |
| HETATM | 29 | N1 | ANP | B | 600 | 6.580 | −62.692 | −43.920 | 1.00 | 50.79 | N |
| HETATM | 30 | C6 | ANP | B | 600 | 5.392 | −62.188 | −44.319 | 1.00 | 50.80 | C |
| HETATM | 31 | N6 | ANP | B | 600 | 4.295 | −62.253 | −43.527 | 1.00 | 50.98 | N |
| HETATM | 1 | MG | MG | B | 700 | 1.653 | −53.618 | −53.006 | 1.00 | 49.55 | MG |
| HETATM | 1 | CA | CA | B | 701 | 19.587 | −77.142 | −8.004 | 1.00 | 46.71 | CA |

### REFERENCES

1. Praefcke, G. J. & McMahon, H. T. The dynamin superfamily: universal membrane tubulation and fission molecules? Nat. Rev. Mol. Cell Biol. 5, 133-147 (2004).
2. Shao, Y. et al. Pincher, a pinocytic chaperone for nerve growth factor/TrkA signaling endosomes. J. Cell Biol. 157, 679-691 (2002).
3. Grant, B. et al. Evidence that RME-1, a conserved C. elegans EH-domain protein, functions in endocytic recycling. Nat. Cell Biol. 3, 573-579 (2001).
4. Caplan, S. et al. A tubular EHD1-containing compartment involved in the recycling of major histocompatibility complex class I molecules to the plasma membrane. EMBO J. 21, 2557-2567 (2002).
5. Sweitzer, S. M. & Hinshaw, J. E. Dynamin undergoes a GTP-dependent conformational change causing vesiculation. Cell 93, 1021-1029(1998).
6. Sever, S., Muhlberg, A. B. & Schmid, S. L. Impairment of dynamin's GAP domain stimulates receptor-mediated endocytosis. Nature 398, 481-486 (1999).
7. Marks, B. et al. GTPase activity of dynamin and resulting conformation change are essential for endocytosis. Nature 410, 231-235 (2001).
8. Roux, A., Uyhazi, K., Frost, A. & De Camilli, P. GTP-dependent twisting of dynamin implicates constriction and tension in membrane fission. Nature 441, 528-531 (2006).
9. Lin, S. X., Grant, B., Hirsh, D. & Maxfield, F. R. Rme-1 regulates the distribution and function of the endocytic recycling compartment in mammalian cells. Nat. Cell Biol. 3, 567-572 (2001).
10. Valdez, G. et al. Pincher-mediated macroendocytosis underlies retrograde signaling by neurotrophin receptors. J. Neurosci. 25, 5236-5247 (2005).
11. Rotem-Yehudar, R., Galperin, E. & Horowitz, M. Association of insulin-like growth factor 1 receptor with EHD1 and SNAP29. J. Biol. Chem. 276, 33054-33060 (2001).
12. Jovic, M., Naslaysky, N., Rapaport, D., Horowitz, M. & Caplan, S. EHD1 regulates betal integrin endosomal transport: effects on focal adhesions, cell spreading and migration. J. Cell Sci. 120, 802-814 (2007).
13. Blume, J. J., Halbach, A., Behrendt, D., Paulsson, M. & Plomann, M. EHD proteins are associated with tubular and vesicular compartments and interact with specific phospholipids. Exp. Cell Res. 313, 219-231 (2007).
14. Park, S. Y. et al. EHD2 interacts with the insulin-responsive glucose transporter (GLUT4) in rat adipocytes and may participate in insulin-induced GLUT4 recruitment. Biochemisny 43, 7552-7562 (2004).
15. Lee, D. W. et al. ATP binding regulates oligomerization and endosome association of RME-1 family proteins. J. Biol. Chem. 280, 17213-17220 (2005).
16. Stowell, M. H., Marks, B., Wigge, P. & McMahon, H. T. Nucleotide-dependent conformational changes in dynamin: evidence for a mechanochemical molecular spring. Nat. Cell Biol. 1, 27-32 (1999).
17. Guilherme, A. et al. EHD2 and the novel EH domain binding protein EHBP 1 couple endocytosis to the actin cytoskeleton. J. Biol. Chem. 279, 10593-10605 (2004).
18. Ghosh, A., Praefcke, G. J., Renault, L., Wittinghofer, A. & Herrmann, C. How guanylate-binding proteins achieve assembly-stimulated processive cleavage of GTP to GMP. Nature 440, 101-104 (2006).
19. Reubold, T. F. et al. Crystal structure of the GTPase domain of rat dynamin 1. Proc. Natl. Acad. Sci. U.S.A. 102, 13093-13098 (2005).
20. Low, H. H. & Lowe, J. A bacterial dynamin-like protein. Nature 444, 766-769 (2006).
21. de Beer, T., Carter, R. E., Lobel-Rice, K. E., Sorkin, A. & Overduin, M. Structure and Asn-Pro-Phe binding pocket of the Eps15 homology domain. Science 281, 1357-1360 (1998).
22. de Beer, T. et al. Molecular mechanism of NPF recognition by EH domains. Nat. Struct. Biol. 7, 1018-1022 (2000).
23. Schmidt, G. et al. Biochemical and biological consequences of changing the specificity of p21ras from guanosine to xanthosine nucleotides. Oncogene 12, 87-96 (1996).
24. Heo, W. D. et al. PI(3,4,5)P3 and PI(4,5)P2 lipids target proteins with polybasic clusters to the plasma membrane. Science 314, 1458-1461 (2006).
25. Prakash, B., Renault, L., Praefcke, G. J., Herrmann, C. & Wittinghofer, A. Triphosphate structure of guanylate-binding protein 1 and implications for nucleotide binding and GTPase mechanism. EMBO J. 19, 4555-4564 (2000).
26. Vetter, I. R. & Wittinghofer, A. The guanine nucleotide-binding switch in three dimensions. Science 294, 1299-1304 (2001).
27. Braun, A. et al. EHD proteins associate with syndapin I and II and such interactions play a crucial role in endosomal recycling. Mol. Biol. Cell 16, 3642-3658 (2005).
28. Egea, P. F. et al. Substrate twinning activates the signal recognition particle and its receptor. Nature 427, 215-21 (2004).
29. Zimmerberg, L & Kozlov, M. M. How proteins produce cellular membrane curvature. Nat. Rev. Mol. Cell Biol. 7, 9-19 (2006).

All publications mentioned in the above specification are herein incorporated by reference. Various modifications and variations of the described aspects and embodiments of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the following claims.

1. A method of identifying a modulator of an EHD family polypeptide, said method comprising
(i) providing a first and second sample of an EHD polypeptide;
(ii) contacting said first sample with a candidate modulator;
(iii)contacting said first and second samples with ATP; and
(iv)monitoring ATP hydrolysis in said first and second samples, wherein a difference between the ATP hydrolysis in said first and second samples identifies said candidate modulator as a modulator of an EHD family polypeptide.

2-46. (canceled)

47. The method according to claim 1 wherein if hydrolysis of ATP is greater in said first sample than in said second sample then the candidate modulator is identified as an enhancer of EHD family polypeptide activity.

48. The method according to claim 1 wherein if hydrolysis of ATP is lower in said first sample than in said second sample then the candidate modulator is identified as an inhibitor of EHD family polypeptide activity.

49. The method according to claim 1 wherein ATP hydrolysis is monitored in the presence of lipid.

50. The method according to claim 4 wherein said lipid is in the form of liposomes.

51. The method according to claim 4 wherein said lipid is in the form of phosphatidylserine (PS) at a final concentration of about 10%.

52. The method according to claim 1 further comprising providing a further sample of EHD family polypeptide, said further sample comprising an EHD family polypeptide bearing a T94A mutation, said further sample being used to determine the reference or background level of spontaneous ATP hydrolysis.

53. The method according to claim 1 further comprising providing a further sample of EHD family polypeptide, said further sample comprising an EHD family polypeptide bearing an I157Q mutation, said further sample being used to determine the reference level of ATP hydrolysis in the absence of lipids.

54. The method according to claim 1 further comprising providing a further reference sample, said further reference sample comprising a dynamin polypeptide together with GTP nucleotide and candidate modulator, said further sample being used to determine whether the candidate modulator has an EHD-specific effect, or whether it is also capable of affecting dynamin GTPase activity.


SEQUENCE LISTING (15 sequences)

SEQ ID NO: 1 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTADCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 2 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGQLSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 3 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPQPTTDCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 4 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGILSGAKQRVSEGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 5 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHDLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 6 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHKLEISREFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 7 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHKLEISDEFSEAIGALRGHEDKIRVVLNKADMVETQQLMRVYGALMXALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 8 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKASFIQYLLEQEVPGSRVGPEPTTDCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 9 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGNLSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 10 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGRLSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 11 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVAGKENKKKQLILKLPVIFAKIQLEHHISPGDFPDCQKMQELLMAHDFTKFHSLKPKLLEALDDMLAQDIAKLMPLLRQEELESVEAGVQGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE

SEQ ID NO: 12 - protein, 8 aa, Artificial Sequence
  oligopeptide from EHD2
PEKPFRKL

SEQ ID NO: 13 - protein, 8 aa, Artificial Sequence
  oligopeptide from EHD2
RMGPFVER

SEQ ID NO: 14 - protein, 8 aa, Artificial Sequence
  oligopeptide from EHD2
KLNPFGNT

SEQ ID NO: 15 - protein, 543 aa, Artificial
  mmEHD2 mutant
MFSWLKKGGARGQRPEAIRTVTSSLKELYRTKLLPLEEHYRFGSFHSPALEDADFDGKPMVLVAGQYSTGKTSFIQYLLEQEVPGSRVGPEPTTDCFVAVMHGETEGTVPGNALVVDPEKPFRKLNPFGNTFLNRFMCAQLPNQVLESISIIDTPGILSGAKQRVSRGYDFPAVLRWFAERVDLIILLFDAHKLEISDEFSEAIGALRGHEDKIRVVLNKADMVETQQLMRVYGALMWALGKVVGTPEVLRVYIGSFWSQPLLVPDNRRLFELEEQDLFRDIQGLPRHAALRKLNDLVKRARLVRVHAYIISYLKKEMPTVFGKENKKKQLILKLPVIFAKIQLEHHISPGDFPDCQKMQELLMAHDFTKFHSLKPKLLEALDDMLAQDIAKLMPLLRQEELESVEAGVAGGAFEGTRMGPFVERGPDEAIEDGEEGSEDDAEWVVTKDKSKYDEIFYNLAPADGKLSGSKAKTWMVGTKLPNSVLGRIWKLSDVDRDGMLDDEEFALASHLIEAKLEGHGLPTNLPRRLVPPSKRRQKGSAE